(19) United States
(12) Patent Application Publication
(10) Pub. No.: US 2024/0360517 A1
(43) Pub. Date: Oct. 31, 2024

(54) SUBTYPING PROSTATE CANCER TO PREDICT RESPONSE TO HORMONE THERAPY

(71) Applicants: Veracyte SD, Inc., San Diego, CA (US); The Regents of the University of Michigan, Ann Arbor, MI (US)

(72) Inventors: Elai DAVICIONI, La Jolla, CA (US); Nicholas ERHO, Vancouver (CA); Shuang G. ZHAO, Ann Arbor, MI (US); Sei-Won Laura CHANG, Ann Arbor, MI (US); Felix Y. FENG, Ann Arbor, MI (US)

(21) Appl. No.: 18/411,722

(22) Filed: Jan. 12, 2024

Related U.S. Application Data
(63) Continuation of application No. 16/492,055, filed on Sep. 6, 2019, now Pat. No. 11,873,532.
(60) Provisional application No. 62/469,174, filed on Mar. 9, 2017.

(51) Int. Cl. C12Q 1/6886; G06N 3/00; G16B 5/00

(52) U.S. Cl. CPC C12Q 1/6886; G06N 3/00; G16B 5/00; C12Q 2600/106; C12Q 2600/112; C12Q 2600/158

(57) ABSTRACT

The present invention relates to methods, systems and kits for the diagnosis, prognosis and the determination of progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

### CROSS-REFERENCE TO RELATED APPLICATIONS

This application is a continuation of U.S. application Ser. No. 16/492,055, filed Sep. 6, 2019, which is a U.S. National Phase under 35 U.S.C. § 371 of International Application No. PCT/US2018/021826, filed on Mar. 9, 2018, which claims benefit of priority of U.S. Application No. 62/469,174, filed Mar. 9, 2017, the entire contents of which is incorporated herein by reference in its entirety.

### INCORPORATION OF SEQUENCE LISTING

The material in the accompanying sequence listing is hereby incorporated by reference into this application. The accompanying sequence listing XML file, named GENDX_017C1, was created on Jan. 11, 2024, and is 524,305 bytes.

### FIELD OF THE INVENTION

The present invention relates to methods, systems and kits for the diagnosis, prognosis and the determination of cancer progression of prostate cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided. The methods and classifiers of the present invention are also useful for predicting response to hormonal therapy (e.g., androgen deprivation therapy).

### BACKGROUND OF THE INVENTION

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, prostate cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if prostate cancer cells spread to a bone (or anywhere else), it can mean that the individual has metastatic prostate cancer.

Standard clinical parameters such as tumor size, grade, lymph node involvement and tumor-node-metastasis (TNM) staging (American Joint Committee on Cancer http://www.cancerstaging.org) may correlate with outcome and serve to stratify patients with respect to (neo) adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment. Thus, provided herein are methods, systems and kits for the diagnosis, prognosis and the determination of cancer progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided. The classifiers of the present invention are useful for identifying prostate cancer patients that will respond to hormone therapy (e.g., androgen deprivation therapy).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to methods, systems and kits for the diagnosis, prognosis and the determination of cancer progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

In one embodiment, the present invention provides a method comprising: providing a biological sample from a prostate cancer subject; detecting the presence or expression level of at least one or more targets selected from Table 8, Table 9 or SEQ ID NOs: 1-1029; and subtyping the prostate cancer in the subject according to a genomic subtyping classifier based on the presence or expression levels of the plurality of targets wherein said subtyping comprises assigning the prostate cancer to one of three subtypes selected from the group consisting of a luminal A subtype, a luminal B subtype, and a basal subtype. In some embodiments, the method further comprises administering androgen deprivation therapy to the subject if the subtyping indicates that the subject has the luminal B subtype and administering an anti-cancer treatment other than the androgen deprivation therapy to the subject if the subtyping indicates that the subject has the luminal A subtype or the basal subtype, wherein the anti-cancer treatment other than androgen deprivation therapy is selected from the group consisting of surgery, chemotherapy, radiation therapy, immunotherapy, biological therapy, neoadjuvant chemotherapy, and photodynamic therapy.

In one embodiment, the present invention provides a method comprising: a) providing a biological sample from a subject having prostate cancer; b) detecting the presence or expression level in the biological sample for a plurality of targets selected from Table 8, Table 9 or SEQ ID NOs: 1-1029 and c) subtyping the prostate cancer in the subject according to a genomic subtyping classifier based on the presence or expression levels of the plurality of targets, wherein said subtyping comprises assigning the prostate cancer to one of three subtypes selected from the group consisting of a luminal A subtype, a luminal B subtype, and a basal subtype.

In one embodiment, the present invention provides a method comprising: a) providing a biological sample from a subject having prostate cancer; b) detecting the presence or expression level in the biological sample for a plurality of targets selected from the group consisting of CDC20; KIF2C; PHGDH; NUF2; CENPF; EXO1; UBE2T; RRM2; MLPH; GPR160; CCNB1; CXXC5; PTTG1; FGFR4; FOXC1; ESR1; ANLN; BLVRA; EGFR; ACTR3B; NAT1; MYC; SFRP1; MELK; BAG1; CEP55; MKI67; TMEM45B; PGR; MDM2; KRT5; FOXA1; ORC6; CDH3; ERBB2; GRB7; CDC6; MAPT; BIRC5; KRT14; KRT17; TYMS; NDC80; SLC39A6; BCL2; CCNE1; MIA; MYBL2; UBE2C; MMP11; TDRD1; CACNA1D; NCALD; HLA-DMB; KCNH8; PDE3B; PLA2G7; CSGALNACT1; PART1; HES1; F3; GPR110; SH3RF; PDE8B; SEPT9; CRISP3; AMD1; KCNG3; PLA1A; MYO6; FRK; SH3YL1; ACER3; C8orf4; GHR; ITPR1; KHDRBS3; NPY; GUCY1A3; ARHGDIB; LAMC2; VWA2; ZNF432; MORN1; CYorf15B; AMPD3; QDPR; HDAC1; KIF16B; GJB1; ITPR3; ZNF615; ANKRD6; APOD; STEAP4; RGS17; MAP7; C22orf36; NKAIN1; CHN2; LRRFIP1; SERGEF; ATP8A2; NDRG1; CDC42SE1; LUZP2; HNF1B; TFAP2A; ANKRD34B; SLC12A2; PRAC; SLC5A4; ACSL3; CD24P4; DNASE2B; SLC22A3; ODC1; SMOC2; UGDH; DSC2; WNK2; RAB3B; FAM198B; KCNC2; SNAP91; FAM65B; AMACR; ZNF385B; CDK19; ARHGAP18; IL5RA; SLC16A1; CNTLN; FKBP10; SLC45A2; CLIP1; HEXB; NEFH; ODZ1; SS18L2; HPGD; FAM3B; MIPEP; NCAPD3; INPP4B; ANPEP; TFF3; IL31RA; EHHADH; RP11-45B20.2; CCDC141; RLN1; ABHD2; SCIN; ALOX15B; MON1B; MME; BANK1; LEPREL1; VGLL3; NPR3; OR4K7P; OR4K6P; POTEB2; RP11; TTN; FAP5; GPR116; RP11.403; and FABP5P7; and c) subtyping the prostate cancer in the subject according to a genomic subtyping classifier based on the presence or expression levels of the plurality of targets, wherein said subtyping comprises assigning the prostate cancer to one of three subtypes selected from the group consisting of a luminal A subtype, a luminal B subtype, and a basal subtype.

In one embodiment, the present invention provides a method comprising: a) providing a biological sample from a subject having prostate cancer and b) detecting the presence or expression level in the biological sample for a plurality of targets selected from Table 8, Table 9 or SEQ ID NOs: 1-1029. In some embodiments, the method further comprises subtyping the prostate cancer in the subject according to a genomic subtyping classifier based on the presence or expression levels of the plurality of targets, wherein said subtyping comprises assigning the prostate cancer to one of three subtypes selected from the group consisting of a luminal A subtype, a luminal B subtype, and a basal subtype.

In some embodiments, the method further comprises administering androgen deprivation therapy to the subject if the subtyping indicates that the subject has the luminal B subtype and administering an anti-cancer treatment other than the androgen deprivation therapy to the subject if the subtyping indicates that the subject has the luminal A subtype or the basal subtype, wherein the anti-cancer treatment other than androgen deprivation therapy is selected from the group consisting of surgery, chemotherapy, radiation therapy, immunotherapy, biological therapy, neoadjuvant chemotherapy, and photodynamic therapy.

In some embodiments, the present invention provides a method comprising: providing a biological sample from a prostate cancer subject; detecting the presence or expression level of at least one or more targets selected from Table 8, Table 9 or SEQ ID NOs: 1-1029; and administering a treatment to the subject, wherein the treatment is selected from the group consisting of surgery, chemotherapy, radiation therapy, immunotherapy/biological therapy, hormonal therapy, and photodynamic therapy. In some embodiments, the treatment is androgen deprivation therapy. In some embodiments, the present invention provides a method of subtyping prostate cancer in a subject, comprising: providing a biological sample comprising prostate cancer cells from the subject, and determining the level of expression or amplification of at least one or more targets selected from Table 8, Table 9 or SEQ ID NOs: 1-1029 using at least one reagent that specifically binds to said targets; wherein the alteration of said expression level provides an indication of the prostate cancer subtype. In some embodiments, the alteration in the expression level of said target is reduced expression of said target. In other embodiments, the alteration in the expression level of said target is increased expression of said target. In yet other embodiments, the level of expression of said target is determined by using a method selected from the group consisting of in situ hybridization, a PCR-based method, an array-based method, an immunohistochemical method, an RNA assay method and an immunoassay method. In other embodiments, the reagent is selected from the group consisting of a nucleic acid probe, one or more nucleic acid primers, and an antibody. In still other embodiments, the target comprises a nucleic acid sequence.

In some embodiments, the present invention also provides a method of diagnosing, prognosing, assessing the risk of recurrence or predicting benefit from therapy in a subject with prostate cancer, comprising: providing a biological sample comprising prostate cancer cells from the subject; assaying an expression level in the biological sample from the subject for a plurality of targets using at least one reagent that specifically binds to said targets, wherein the plurality of targets comprises one or more targets selected from Table 8, Table 9 or SEQ ID NOs: 1-1029; and diagnosing, prognosing, assessing the risk of recurrence or predicting benefit from therapy in the subject based on the expression levels of the plurality of targets. In some embodiments, the expression level of the target is reduced expression of the target. In other embodiments, the expression level of said target is increased expression of said target. In yet other embodiments, the level of expression of said target is determined by using a method selected from the group consisting of in situ hybridization, a PCR-based method, an array-based method, an immunohistochemical method, an RNA assay method and an immunoassay method. In other embodiments, the reagent is selected from the group consisting of a nucleic acid probe, one or more nucleic acid primers, and an antibody. In other embodiments, the target comprises a nucleic acid sequence.

In some embodiments, the present invention provides a system for analyzing a cancer, comprising, a probe set comprising a plurality of target sequences, wherein the plurality of target sequences hybridizes to one or more targets selected from Table 8, Table 9 or SEQ ID NOs: 1-1029; or the plurality of target sequences comprises one or more targets selected from Table 8, Table 9 or SEQ ID NOs: 1-1029; and a computer model or algorithm for analyzing an expression level and/or expression profile of the target hybridized to the probe in a sample from a subject suffering from prostate cancer. In some embodiments, the method further comprises a label that specifically binds to the target, the probe, or a combination thereof.

In some embodiments, the present invention provides a method comprising: (a) providing a biological sample from a subject with prostate cancer; (b) detecting the presence or expression level in the biological sample for a plurality of targets, wherein the plurality of targets comprises one or more targets selected from Table 8, Table 9 or SEQ ID NOs: 1-1029; (c) subtyping the prostate cancer in the subject based on the presence or expression levels of the plurality of targets; and (d) administering a treatment to the subject, wherein the treatment is selected from the group consisting of surgery, chemotherapy, radiation therapy, immunotherapy/biological therapy, hormonal therapy, and photodynamic therapy. In some embodiments, the present invention provides a method of treating a subject with prostate cancer, comprising: providing a biological sample comprising prostate cancer cells from the subject; determining the level of expression or amplification of at least one or more targets selected from Table 8, Table 9 or SEQ ID NOs: 1-1029 using at least one reagent that specifically binds to said targets; subtyping the prostate cancer based on the level of expression or amplification of the at least one or more targets; and prescribing a treatment regimen based on the prostate cancer subtype. In some embodiments, the prostate cancer subtype is selected from the group consisting of luminal A, luminal B, and basal.

In some embodiments, the present invention provides a kit for analyzing a prostate cancer, comprising, a probe set comprising a plurality of target sequences, wherein the plurality of target sequences comprises at least one target sequence listed in Table 8, Table 9 or SEQ ID NOs: 1-1029; and a computer model or algorithm for analyzing an expression level and/or expression profile of the target sequences in a sample. In some embodiments, the method further comprises a computer model or algorithm for correlating the expression level or expression profile with disease state or outcome. In other embodiments, the method further comprises a computer model or algorithm for designating a treatment modality for the individual. In yet other embodiments, the method further comprises a computer model or algorithm for normalizing expression level or expression profile of the target sequences. In some embodiments, the method further comprises sequencing the plurality of targets. In some embodiments, the method further comprises hybridizing the plurality of targets to a solid support. In some embodiments, the solid support is a bead or array. In some embodiments, assaying the expression level of a plurality of targets may comprise the use of a probe set. In some embodiments, assaying the expression level may comprise the use of a classifier. The classifier may comprise a probe selection region (PSR). In some embodiments, the classifier may comprise the use of an algorithm. The algorithm may comprise a machine learning algorithm. In some embodiments, assaying the expression level may also comprise sequencing the plurality of targets.

Further disclosed herein methods for molecular subtyping of prostate cancer, wherein the subtypes have an AUC value of at least about 0.40 to predict patient outcomes. In some embodiments, patient outcomes are selected from the group consisting of biochemical recurrence (BCR), metastasis (MET) and prostate cancer death (PCSM) after radical prostatectomy. The AUC of the subtype may be at least about 0.40, 0.45, 0.50, 0.55, 0.60, 0.61, 0.62, 0.63, 0.64, 0.65, 0.66, 0.67, 0.68, 0.69, 0.70 or more.

Further disclosed herein is a method for subtyping a prostate cancer, comprising determining the level of expression or amplification of at least one or more targets of the present invention, wherein the significance of the expression level of the one or more targets is based on one or more metrics selected from the group comprising T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) and Multivariable Analysis Hazard Ratio P-value (mvaHRPval). The molecular subtypes of the present invention are useful for predicting clinical characteristics of subjects with prostate cancer. In some embodiments, the clinical characteristics are selected from the group consisting of seminal vesical invasion (SVI), lymph node invasion (LNI), prostate-specific antigen (PSA), and gleason score (GS).

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1B set forth data showing PAM50 clustering and clinical outcomes in prostate cancer. (A) The PAM50 genes cluster prostate cancer samples into three subtypes, luminal A (dark blue), luminal B (light blue) and basal (red), in the pooled prostate cancer cohorts (MCI, II, CC, TJU, JHU, DVA) using hierarchical clustering of the genes. Each column represents a patient sample and each row represents a gene. (B) Kaplan-Meier curves showing that the PAM50 clusters risk stratify bRFS (biochemical recurrence-free survival), DMFS (distant metastasis-free survival), PCSS (prostate cancer specific survival), and OS (overall survival).

FIGS. 2A-2C set forth data showing basal and luminal subtypes are associated with basal and luminal lineage markers.

FIGS. 3A-3D set forth data showing Prospective validation in GRID. PAM50 clusters in a prospective validation cohort of 2215 prostate cancer samples run on a commercial clinical platform (A). AR expression increases in luminal samples (B), the basal lineage CD49f signature in the basal subtype (C), and NKX3-1 and KRT18 expression in the luminal subtypes (D). Bar-plots show the mean with standard error of median centered gene expression, and P-values are from ANOVA.

FIGS. 4A-4C set forth data showing predicted response in ADT. A matched cohort was obtained from the MCI and MCII cohorts which matched 1:2 ADT treated and untreated patients based on Gleason, PSA, LNI, ECE, SVI, and SMS resulting in 315 total patients (A). Kaplan-Meier curves are shown for the luminal B and non-luminal B patients, which group the luminal A and basal patients (B). A bar plot (C) is shown comparing the 10-year metastasis rates for treated and untreated patients in the luminal B and non-luminal B patients, with the interaction term Wald p=0.006.

FIG. 5 sets forth data showing Heatmaps depicting the PAM50 subtypes (each column represents a sample, each row represents a gene, dark blue=Luminal A, light blue=Luminal B, red=Basal) with genes in the same order as shown in FIG. 1A.

FIG. 6 sets forth data showing differences in genetic signatures for breast and prostate cancer for the markers of a genomic classifier of the present invention. A heatmap of the PAM50 clusters in the breast cohort from Parker et al. are shown for basal, luminal A, and luminal B. Boxplots demonstrate that in breast cancer, ER is higher in luminal versus basal, and that PR is highest in luminal A. A heatmap of the PAM50 clusters in prostate cancer is shown (MCI, II, CC, TJU, JHU, DVA) with the same order of genes as displayed for the breast cancer data. Boxplots show that ER does not demonstrate the same differences between luminal and basal as in breast, but PR does show lower expression in luminal B compared to luminal A as in breast cancer.

FIG. 7 sets forth data showing luminal subtypes are associated with MYC and KRAS.

FIG. 8 sets forth data showing PAM50 proliferation score across subtypes. Box plots of the PAM50 proliferation score across the basal, luminal A and luminal B subtypes within the retrospective and prospective cohorts. ANOVA p-value<0.001 for both cohorts.

FIG. 9 sets forth data showing survival outcomes for the matched cohort, separating patients receiving adjuvant and salvage ADT. Kaplan-Meier curves are shown to visualize effect of adjuvant and salvage ADT separately within the Luminal B and non-Luminal B patients. Patients receiving adjuvant ADT and their matched no ADT samples. Patients receiving salvage ADT and their matched no ADT samples.

FIG. 10 sets forth data showing Decipher and mCCP are not predictive for response to post-operative ADT in a matched cohort.

### DETAILED DESCRIPTION OF THE INVENTION

The present invention discloses systems and methods for diagnosing, predicting, and/or monitoring the status or outcome of a prostate cancer in a subject using expression-based analysis of a plurality of targets. Generally, the method comprises (a) optionally providing a sample from a subject; (b) assaying the expression level for a plurality of targets in the sample; and (c) diagnosing, predicting and/or monitoring the status or outcome of a prostate cancer based on the expression level of the plurality of targets.

Assaying the expression level for a plurality of targets in the sample may comprise applying the sample to a microarray. In some instances, assaying the expression level may comprise the use of an algorithm. The algorithm may be used to produce a classifier. Alternatively, the classifier may comprise a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

In some instances, the plurality of targets comprises one or more targets selected from Table 8, Table 9 or SEQ ID NOs: 1-1029. In some instances, the plurality of targets comprises at least about 2, at least about 3, at least about 4, at least about 5, at least about 6, at least about 7, at least about 8, at least about 9, at least about 10, at least about 20, at least about 30, at least about 40, or at least about 50 targets selected from Table 8, Table 9 or SEQ ID NOs: 1-1029.

Further disclosed herein are methods for subtyping prostate cancer. Generally, the method comprises: (a) providing a sample comprising prostate cancer cells from a subject; (b) assaying the expression level for a plurality of targets in the sample; and (c) subtyping the cancer based on the expression level of the plurality of targets. In some instances, the plurality of targets comprises one or more targets selected from Table 8, Table 9 or SEQ ID NOs: 1-1029. In some instances, the plurality of targets comprises at least about 2, at least about 3, at least about 4, at least about 5, at least about 6, at least about 7, at least about 8, at least about 9, at least about 10, at least about 20, at least about 30, at least about 40, or at least about 50 targets selected from Table 8, Table 9 or SEQ ID NOs: 1-1029.

In some instances, subtyping the prostate cancer comprises determining whether the cancer would respond to an anti-cancer therapy. Alternatively, subtyping the prostate cancer comprises identifying the cancer as non-responsive to an anti-cancer therapy. Optionally, subtyping the prostate cancer comprises identifying the cancer as responsive to an anti-cancer therapy.

Before the present invention is described in further detail, it is to be understood that this invention is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present invention.

### Targets

The methods disclosed herein often comprise assaying the expression level of a plurality of targets. The plurality of targets may comprise coding targets and/or non-coding targets of a protein-coding gene or a non protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets may be differentially expressed. In some instances, a plurality of probe selection regions (PSRs) is differentially expressed.

In some instances, the plurality of targets comprises one or more targets selected from at least about 10, at least about 20, at least about 30, at least about 40, or at least about 50 targets selected from Table 8, Table 9 or SEQ ID NOs: 1-1029. In some instances, the plurality of targets comprises at least about 2, at least about 3, at least about 4, at least about 5, at least about 6, at least about 7, at least about 8, at least about 9, at least about 10, at least about 20, at least about 30, at least about 40, or at least about 50 targets selected from Table 8, Table 9 or SEQ ID NOs: 1-1029.

In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15, 20, 25, 30, 40, 50 or more targets selected from a Random Forest (RF) classifier. The RF classifier may be an RF2, and RF3, or an RF4 classifier. The RF classifier may be an RF50 classifier (e.g., a Random Forest classifier with 50 targets).

A RF classifier of the present invention may comprise two or more targets comprising two or more targets selected from Table 8, Table 9 or SEQ ID NOs: 1-1029.

The plurality of targets may comprise one or more targets selected from an SVM classifier. The plurality of targets may comprise 2, 3, 4, 5, 6, 7, 8, 9, 10, 11, 12, 13, 14, 15, 20, 25, 30, 40, 50 or more targets selected from an SVM classifier. The plurality of targets may comprise 12, 13, 14, 15, 17, 20, 22, 25, 27, 30, 40, 50 or more targets selected from an SVM classifier. The plurality of targets may comprise 32, 35, 37, 40, 43, 45, 47, 50 or more targets selected from an SVM classifier. The SVM classifier may be an SVM2 classifier.

A SVM classifier of the present invention may comprise two or more targets comprising two or more targets selected from Table 8, Table 9 or SEQ ID NOs: 1-1029.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50 or more targets selected from a KNN classifier.

The KNN classifier may be a KNN50 classifier. A KNN classifier of the present invention may comprise fifty or more targets comprising fifty or more targets selected from Table 8, Table 9 or SEQ ID NOs: 1-1029.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50 or more targets selected from a NB classifier.

The NB classifier may be a NB2 classifier. An NB classifier of the present invention may comprise two or more targets comprising two or more targets selected from Table 8, Table 9 or SEQ ID NOs: 1-1029.

The plurality of targets may comprise one or more targets selected from a recursive Partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50 or more targets selected from an Rpart classifier.

The Rpart classifier may be an Rpart2 classifier. An Rpart classifier of the present invention may comprise two or more targets comprising two or more targets selected from Table 8, Table 9 or SEQ ID NOs: 1-1029.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15, 20, 30, 40, 50 or more targets selected from a high dimensional discriminate analysis (HDDA) classifier.

The plurality of targets may comprise one or more targets selected from CDC20, KIF2C, PHGDH, NUF2, CENPF, EXO1, UBE2T, RRM2, MLPH, GPR160, CCNB1, CXXC5, PTTG1, FGFR4, FOXC1, ESR1, ANLN, BLVRA, EGFR, ACTR3B, NAT1, MYC, SFRP1, MELK, BAG1, CEP55, MKI67, TMEM45B, PGR, MDM2, KRT5, FOXA1, ORC6, CDH3, ERBB2, GRB7, CDC6, MAPT, BIRC5, KRT14, KRT17, TYMS, NDC80, SLC39A6, BCL2, CCNE1, MIA, MYBL2, UBE2C, and MMP11; CDC20; CDC20 and KIF2C; CDC20 and PHGDH; CDC20 and NUF2; CDC20 and CENPF; CDC20 and EXO1; CDC20 and UBE2T; CDC20 and RRM2; CDC20 and MLPH; CDC20 and GPR160; CDC20 and CCNB1; CDC20 and CXXC5; CDC20 and PTTG1; CDC20 and FGFR4; CDC20 and FOXC1; CDC20 and ESR1; CDC20 and ANLN; CDC20 and BLVRA; CDC20 and EGFR; CDC20 and ACTR3B; CDC20 and NAT1; CDC20 and MYC; CDC20 and SFRP1; CDC20 and MELK; CDC20 and BAG1; CDC20 and CEP55; CDC20 and MKI67; CDC20 and TMEM45B; CDC20 and PGR; CDC20 and MDM2; CDC20 and KRT5; CDC20 and FOXA1; CDC20 and ORC6; CDC20 and CDH3; CDC20 and ERBB2; CDC20 and GRB7; CDC20 and CDC6; CDC20 and MAPT; CDC20 and BIRC5; CDC20 and KRT14; CDC20 and KRT17; CDC20 and TYMS; CDC20 and NDC80; CDC20 and SLC39A6; CDC20 and BCL2; CDC20 and CCNE1; CDC20 and MIA; CDC20 and MYBL2; CDC20 and UBE2C; CDC20 and MMP11; CDC20, KIF2C and PHGDH; CDC20, KIF2C and NUF2; CDC20, KIF2C and CENPF; CDC20, KIF2C and EXO1; CDC20, KIF2C and UBE2T; CDC20, KIF2C and RRM2; CDC20, KIF2C and MLPH; CDC20, KIF2C and GPR160; CDC20, KIF2C and CCNB1; CDC20, KIF2C and CXXC5; CDC20, KIF2C and PTTG1; CDC20, KIF2C and FGFR4; CDC20, KIF2C and FOXC1; CDC20, KIF2C and ESR1; CDC20, KIF2C and ANLN; CDC20, KIF2C and BLVRA; CDC20, KIF2C and EGFR; CDC20, KIF2C and ACTR3B; CDC20, KIF2C and NAT1; CDC20, KIF2C and MYC; CDC20, KIF2C and SFRP1; CDC20, KIF2C and MELK; CDC20, KIF2C and BAG1; CDC20, KIF2C and CEP55; CDC20, KIF2C and MKI67; CDC20, KIF2C and TMEM45B; CDC20, KIF2C and PGR; CDC20, KIF2C and MDM2; CDC20, KIF2C and KRT5; CDC20, KIF2C and FOXA1; CDC20, KIF2C and ORC6; CDC20, KIF2C and CDH3; CDC20, KIF2C and ERBB2; CDC20, KIF2C and GRB7; CDC20, KIF2C and CDC6; CDC20, KIF2C and MAPT; CDC20, KIF2C and BIRC5; CDC20, KIF2C and KRT14; CDC20, KIF2C and KRT17; CDC20, KIF2C and TYMS; CDC20, KIF2C and NDC80; CDC20, KIF2C and SLC39A6; CDC20, KIF2C and BCL2; CDC20, KIF2C and CCNE1; CDC20, KIF2C and MIA; CDC20, KIF2C and MYBL2; CDC20, KIF2C and UBE2C; CDC20, KIF2C and MMP11; CDC20, KIF2C, PHGDH and NUF2; CDC20, KIF2C, PHGDH and CENPF; CDC20, KIF2C, PHGDH and EXO1; CDC20, KIF2C, PHGDH and UBE2T; CDC20, KIF2C, PHGDH and RRM2; CDC20, KIF2C, PHGDH and MLPH; CDC20, KIF2C, PHGDH and GPR160; CDC20, KIF2C, PHGDH and CCNB1; CDC20, KIF2C, PHGDH and CXXC5; CDC20, KIF2C, PHGDH and PTTG1; CDC20, KIF2C, PHGDH and FGFR4; CDC20, KIF2C, PHGDH and FOXC1; CDC20, KIF2C, PHGDH and ESR1; CDC20, KIF2C, PHGDH and ANLN; CDC20, KIF2C, PHGDH and BLVRA; CDC20, KIF2C, PHGDH and EGFR; CDC20, KIF2C, PHGDH and ACTR3B; CDC20, KIF2C, PHGDH and NAT1; CDC20, KIF2C, PHGDH and MYC; CDC20, KIF2C, PHGDH and SFRP1; CDC20, KIF2C, PHGDH and MELK; CDC20, KIF2C, PHGDH and BAG1; CDC20, KIF2C, PHGDH and CEP55; CDC20, KIF2C, PHGDH and MKI67; CDC20, KIF2C, PHGDH and TMEM45B; CDC20, KIF2C, PHGDH and PGR; CDC20, KIF2C, PHGDH and MDM2; CDC20, KIF2C, PHGDH and KRT5; CDC20, KIF2C, PHGDH and FOXA1; CDC20, KIF2C, PHGDH and ORC6; CDC20, KIF2C, PHGDH and CDH3; CDC20, KIF2C, PHGDH and ERBB2; CDC20, KIF2C, PHGDH and GRB7; CDC20, KIF2C, PHGDH and CDC6; CDC20, KIF2C, PHGDH and MAPT; CDC20, KIF2C, PHGDH and BIRC5; CDC20, KIF2C, PHGDH and KRT14; CDC20, KIF2C, PHGDH and KRT17; CDC20, KIF2C, PHGDH and TYMS; CDC20, KIF2C, PHGDH and NDC80; CDC20, KIF2C, PHGDH and SLC39A6; CDC20, KIF2C, PHGDH and BCL2; CDC20, KIF2C, PHGDH and CCNE1; CDC20, KIF2C, PHGDH and MIA; CDC20, KIF2C, PHGDH and MYBL2; CDC20, KIF2C, PHGDH and UBE2C; CDC20, KIF2C, PHGDH and MMP11; KIF2C; KIF2C and PHGDH; KIF2C and NUF2; KIF2C and CENPF; KIF2C and EXO1; KIF2C and UBE2T; KIF2C and RRM2; KIF2C and MLPH; KIF2C and GPR160; KIF2C and CCNB1; KIF2C and CXXC5; KIF2C and PTTG1; KIF2C and FGFR4; KIF2C and FOXC1; KIF2C and ESR1; KIF2C and ANLN; KIF2C and BLVRA; KIF2C and EGFR; KIF2C and ACTR3B; KIF2C and NAT1; KIF2C and MYC; KIF2C and SFRP1; KIF2C and MELK; KIF2C and BAG1; KIF2C and CEP55; KIF2C and MKI67; KIF2C and TMEM45B; KIF2C and PGR; KIF2C and MDM2; KIF2C and KRT5; KIF2C and FOXA1; KIF2C and ORC6; KIF2C and CDH3; KIF2C and ERBB2; KIF2C and GRB7; KIF2C and CDC6; KIF2C and MAPT; KIF2C and BIRC5; KIF2C and KRT14; KIF2C and KRT17; KIF2C and TYMS; KIF2C and NDC80; KIF2C and SLC39A6; KIF2C and BCL2; KIF2C and CCNE1; KIF2C and MIA; KIF2C and MYBL2; KIF2C and UBE2C; KIF2C and MMP11; KIF2C, PHGDH and NUF2; KIF2C, PHGDH and CENPF; KIF2C, PHGDH and EXO1; KIF2C, PHGDH and UBE2T; KIF2C, PHGDH and RRM2; KIF2C, PHGDH and MLPH; KIF2C, PHGDH and GPR160; KIF2C, PHGDH and CCNB1; KIF2C, PHGDH and CXXC5; KIF2C, PHGDH and PTTG1; KIF2C, PHGDH and FGFR4; KIF2C, PHGDH and FOXC1; KIF2C, PHGDH and ESR1; KIF2C, PHGDH and ANLN; KIF2C, PHGDH and BLVRA; KIF2C, PHGDH and EGFR; KIF2C, PHGDH and ACTR3B; KIF2C, PHGDH and NAT1; KIF2C, PHGDH and MYC; KIF2C, PHGDH and SFRP1; KIF2C, PHGDH and MELK; KIF2C, PHGDH and BAG1; KIF2C, PHGDH and CEP55; KIF2C, PHGDH and MKI67; KIF2C, PHGDH and TMEM45B; KIF2C, PHGDH and PGR; KIF2C, PHGDH and MDM2; KIF2C, PHGDH and KRT5; KIF2C, PHGDH and FOXA1; KIF2C, PHGDH and ORC6; KIF2C, PHGDH and CDH3; KIF2C, PHGDH and ERBB2; KIF2C, PHGDH and GRB7; KIF2C, PHGDH and CDC6; KIF2C, PHGDH and MAPT; KIF2C, PHGDH and BIRC5; KIF2C, PHGDH and KRT14; KIF2C, PHGDH and KRT17; KIF2C, PHGDH and TYMS; KIF2C, PHGDH and NDC80; KIF2C, PHGDH and SLC39A6; KIF2C, PHGDH and BCL2; KIF2C, PHGDH and CCNE1; KIF2C, PHGDH and MIA; KIF2C, PHGDH and MYBL2; KIF2C, PHGDH and UBE2C; KIF2C, PHGDH and MMP11; KIF2C, PHGDH, NUF2 and CENPF; KIF2C, PHGDH, NUF2 and EXO1; KIF2C, PHGDH, NUF2 and UBE2T; KIF2C, PHGDH, NUF2 and RRM2; KIF2C, PHGDH, NUF2 and MLPH; KIF2C, PHGDH, NUF2 and GPR160; KIF2C, PHGDH, NUF2 and CCNB1; KIF2C, PHGDH, NUF2 and CXXC5; KIF2C, PHGDH, NUF2 and PTTG1; KIF2C, PHGDH, NUF2 and FGFR4; KIF2C, PHGDH, NUF2 and FOXC1; KIF2C, PHGDH, NUF2 and ESR1; KIF2C, PHGDH, NUF2 and ANLN; KIF2C, PHGDH, NUF2 and BLVRA; KIF2C, PHGDH, NUF2 and EGFR; KIF2C, PHGDH, NUF2 and ACTR3B; KIF2C, PHGDH, NUF2 and NAT1; KIF2C, PHGDH, NUF2 and MYC; KIF2C, PHGDH, NUF2 and SFRP1; KIF2C, PHGDH, NUF2 and MELK; KIF2C, PHGDH, NUF2 and BAG1; KIF2C, PHGDH, NUF2 and CEP55; KIF2C, PHGDH, NUF2 and MKI67; KIF2C, PHGDH, NUF2 and TMEM45B; KIF2C, PHGDH, NUF2 and PGR; KIF2C, PHGDH, NUF2 and MDM2; KIF2C, PHGDH, NUF2 and KRT5; KIF2C, PHGDH, NUF2 and FOXA1; KIF2C, PHGDH, NUF2 and ORC6; KIF2C, PHGDH, NUF2 and CDH3; KIF2C, PHGDH, NUF2 and ERBB2; KIF2C, PHGDH, NUF2 and GRB7; KIF2C, PHGDH, NUF2 and CDC6; KIF2C, PHGDH, NUF2 and MAPT; KIF2C, PHGDH, NUF2 and BIRC5; KIF2C, PHGDH, NUF2 and KRT14; KIF2C, PHGDH, NUF2 and KRT17; KIF2C, PHGDH, NUF2 and TYMS; KIF2C, PHGDH, NUF2 and NDC80; KIF2C, PHGDH, NUF2 and SLC39A6; KIF2C, PHGDH, NUF2 and BCL2; KIF2C, PHGDH, NUF2 and CCNE1; KIF2C, PHGDH, NUF2 and MIA; KIF2C, PHGDH, NUF2 and MYBL2; KIF2C, PHGDH, NUF2 and UBE2C; KIF2C, PHGDH, NUF2 and MMP11; PHGDH; PHGDH and NUF2; PHGDH and CENPF; PHGDH and EXO1; PHGDH and UBE2T; PHGDH and RRM2; PHGDH and MLPH; PHGDH and GPR160; PHGDH and CCNB1; PHGDH and CXXC5; PHGDH and PTTG1; PHGDH and FGFR4; PHGDH and FOXC1; PHGDH and ESR1; PHGDH and ANLN; PHGDH and BLVRA; PHGDH and EGFR; PHGDH and ACTR3B; PHGDH and NAT1; PHGDH and MYC; PHGDH and SFRP1; PHGDH and MELK; PHGDH and BAG1; PHGDH and CEP55; PHGDH and MKI67; PHGDH and TMEM45B; PHGDH and PGR; PHGDH and MDM2; PHGDH and KRT5; PHGDH and FOXA1; PHGDH and ORC6; PHGDH and CDH3; PHGDH and ERBB2; PHGDH and GRB7; PHGDH and CDC6; PHGDH and MAPT; PHGDH and BIRC5; PHGDH and KRT14; PHGDH and KRT17; PHGDH and TYMS; PHGDH and NDC80; PHGDH and SLC39A6; PHGDH and BCL2; PHGDH and CCNE1; PHGDH and MIA; PHGDH and MYBL2; PHGDH and UBE2C; PHGDH and MMP11; PHGDH, NUF2 and CENPF; PHGDH, NUF2 and EXO1; PHGDH, NUF2 and UBE2T; PHGDH, NUF2 and RRM2; PHGDH, NUF2 and MLPH; PHGDH, NUF2 and GPR160; PHGDH, NUF2 and CCNB1; PHGDH, NUF2 and CXXC5; PHGDH, NUF2 and PTTG1; PHGDH, NUF2 and FGFR4; PHGDH, NUF2 and FOXC1; PHGDH, NUF2 and ESR1; PHGDH, NUF2 and ANLN; PHGDH, NUF2 and BLVRA; PHGDH, NUF2 and EGFR; PHGDH, NUF2 and ACTR3B; PHGDH, NUF2 and NAT1; PHGDH, NUF2 and MYC; PHGDH, NUF2 and SFRP1; PHGDH, NUF2 and MELK; PHGDH, NUF2 and BAG1; PHGDH, NUF2 and CEP55; PHGDH, NUF2 and MKI67; PHGDH, NUF2 and TMEM45B; PHGDH, NUF2 and PGR; PHGDH, NUF2 and MDM2; PHGDH, NUF2 and KRT5; PHGDH, NUF2 and FOXA1; PHGDH, NUF2 and ORC6; PHGDH, NUF2 and CDH3; PHGDH, NUF2 and ERBB2; PHGDH, NUF2 and GRB7; PHGDH, NUF2 and CDC6; PHGDH, NUF2 and MAPT; PHGDH, NUF2 and BIRC5; PHGDH, NUF2 and KRT14; PHGDH, NUF2 and KRT17; PHGDH, NUF2 and TYMS; PHGDH, NUF2 and NDC80; PHGDH, NUF2 and SLC39A6; PHGDH, NUF2 and BCL2; PHGDH, NUF2 and CCNE1; PHGDH, NUF2 and MIA; PHGDH, NUF2 and MYBL2; PHGDH, NUF2 and UBE2C; PHGDH, NUF2 and MMP11; PHGDH, NUF2, CENPF and EXO1; PHGDH, NUF2, CENPF and UBE2T; PHGDH, NUF2, CENPF and RRM2; PHGDH, NUF2, CENPF and MLPH; PHGDH, NUF2, CENPF and GPR160; PHGDH, NUF2, CENPF and CCNB1; PHGDH, NUF2, CENPF and CXXC5; PHGDH, NUF2, CENPF and PTTG1; PHGDH, NUF2, CENPF and FGFR4; PHGDH, NUF2, CENPF and FOXC1; PHGDH, NUF2, CENPF and ESR1; PHGDH, NUF2, CENPF and ANLN; PHGDH, NUF2, CENPF and BLVRA; PHGDH, NUF2, CENPF and EGFR; PHGDH, NUF2, CENPF and ACTR3B; PHGDH, NUF2, CENPF and NAT1; PHGDH, NUF2, CENPF and MYC; PHGDH, NUF2, CENPF and SFRP1; PHGDH, NUF2, CENPF and MELK; PHGDH, NUF2, CENPF and BAG1; PHGDH, NUF2, CENPF and CEP55; PHGDH, NUF2, CENPF and MKI67; PHGDH, NUF2, CENPF and TMEM45B; PHGDH, NUF2, CENPF and PGR; PHGDH, NUF2, CENPF and MDM2; PHGDH, NUF2, CENPF and KRT5; PHGDH, NUF2, CENPF and FOXA1; PHGDH, NUF2, CENPF and ORC6; PHGDH, NUF2, CENPF and CDH3; PHGDH, NUF2, CENPF and ERBB2; PHGDH, NUF2, CENPF and GRB7; PHGDH, NUF2, CENPF and CDC6; PHGDH, NUF2, CENPF and MAPT; PHGDH, NUF2, CENPF and BIRC5; PHGDH, NUF2, CENPF and KRT14; PHGDH, NUF2, CENPF and KRT17; PHGDH, NUF2, CENPF and TYMS; PHGDH, NUF2, CENPF and NDC80; PHGDH, NUF2, CENPF and SLC39A6; PHGDH, NUF2, CENPF and BCL2; PHGDH, NUF2, CENPF and CCNE1; PHGDH, NUF2, CENPF and MIA; PHGDH, NUF2, CENPF and MYBL2; PHGDH, NUF2, CENPF and UBE2C; PHGDH, NUF2, CENPF and MMP11; NUF2; NUF2 and CENPF; NUF2 and EXO1; NUF2 and UBE2T; NUF2 and RRM2; NUF2 and MLPH; NUF2 and GPR160; NUF2 and CCNB1; NUF2 and CXXC5; NUF2 and PTTG1; NUF2 and FGFR4; NUF2 and FOXC1; NUF2 and ESR1; NUF2 and ANLN; NUF2 and BLVRA; NUF2 and EGFR; NUF2 and ACTR3B; NUF2 and NAT1; NUF2 and MYC; NUF2 and SFRP1; NUF2 and MELK; NUF2 and BAG1; NUF2 and CEP55; NUF2 and MKI67; NUF2 and TMEM45B; NUF2 and PGR; NUF2 and MDM2; NUF2 and KRT5; NUF2 and FOXA1; NUF2 and ORC6; NUF2 and CDH3; NUF2 and ERBB2; NUF2 and GRB7; NUF2 and CDC6; NUF2 and MAPT; NUF2 and BIRC5; NUF2 and KRT14; NUF2 and KRT17; NUF2 and TYMS; NUF2 and NDC80; NUF2 and SLC39A6; NUF2 and BCL2; NUF2 and CCNE1; NUF2 and MIA; NUF2 and MYBL2; NUF2 and UBE2C; NUF2 and MMP11; NUF2, CENPF and EXO1; NUF2, CENPF and UBE2T; NUF2, CENPF and RRM2; NUF2, CENPF and MLPH; NUF2, CENPF and GPR160; NUF2, CENPF and CCNB1; NUF2, CENPF and CXXC5; NUF2, CENPF and PTTG1; NUF2, CENPF and FGFR4; NUF2, CENPF and FOXC1; NUF2, CENPF and ESR1; NUF2, CENPF and ANLN; NUF2, CENPF and BLVRA; NUF2, CENPF and EGFR; NUF2, CENPF and ACTR3B; NUF2, CENPF and NAT1; NUF2, CENPF and MYC; NUF2, CENPF and SFRP1; NUF2, CENPF and MELK; NUF2, CENPF and BAG1; NUF2, CENPF and CEP55; NUF2, CENPF and MKI67; NUF2, CENPF and TMEM45B; NUF2, CENPF and PGR; NUF2, CENPF and MDM2; NUF2, CENPF and KRT5; NUF2, CENPF and FOXA1; NUF2, CENPF and ORC6; NUF2, CENPF and CDH3; NUF2, CENPF and ERBB2; NUF2, CENPF and GRB7; NUF2, CENPF and CDC6; NUF2, CENPF and MAPT; NUF2, CENPF and BIRC5; NUF2, CENPF and KRT14; NUF2, CENPF and KRT17; NUF2, CENPF and TYMS; NUF2, CENPF and NDC80; NUF2, CENPF and SLC39A6; NUF2, CENPF and BCL2; NUF2, CENPF and CCNE1; NUF2, CENPF and MIA; NUF2, CENPF and MYBL2; NUF2, CENPF and UBE2C; NUF2, CENPF and MMP11; NUF2, CENPF, EXO1 and UBE2T; NUF2, CENPF, EXO1 and RRM2; NUF2, CENPF, EXO1 and MLPH; NUF2, CENPF, EXO1 and GPR160; NUF2, CENPF, EXO1 and CCNB1; NUF2, CENPF, EXO1 and CXXC5; NUF2, CENPF, EXO1 and PTTG1; NUF2, CENPF, EXO1 and FGFR4; NUF2, CENPF, EXO1 and FOXC1; NUF2, CENPF, EXO1 and ESR1; NUF2, CENPF, EXO1 and ANLN; NUF2, CENPF, EXO1 and BLVRA; NUF2, CENPF, EXO1 and EGFR; NUF2, CENPF, EXO1 and ACTR3B; NUF2, CENPF, EXO1 and NAT1; NUF2, CENPF, EXO1 and MYC; NUF2, CENPF, EXO1 and SFRP1; NUF2, CENPF, EXO1 and MELK; NUF2, CENPF, EXO1 and BAG1; NUF2, CENPF, EXO1 and CEP55; NUF2, CENPF, EXO1 and MKI67; NUF2, CENPF, EXO1 and TMEM45B; NUF2, CENPF, EXO1 and PGR; NUF2, CENPF, EXO1 and MDM2; NUF2, CENPF, EXO1 and KRT5; NUF2, CENPF, EXO1 and FOXA1; NUF2, CENPF, EXO1 and ORC6; NUF2, CENPF, EXO1 and CDH3; NUF2, CENPF, EXO1 and ERBB2; NUF2, CENPF, EXO1 and GRB7; NUF2, CENPF, EXO1 and CDC6; NUF2, CENPF, EXO1 and MAPT; NUF2, CENPF, EXO1 and BIRC5; NUF2, CENPF, EXO1 and KRT14; NUF2, CENPF, EXO1 and KRT17; NUF2, CENPF, EXO1 and TYMS; NUF2, CENPF, EXO1 and NDC80; NUF2, CENPF, EXO1 and SLC39A6; NUF2, CENPF, EXO1 and BCL2; NUF2, CENPF, EXO1 and CCNE1; NUF2, CENPF, EXO1 and MIA; NUF2, CENPF, EXO1 and MYBL2; NUF2, CENPF, EXO1 and UBE2C; NUF2, CENPF, EXO1 and MMP11; CENPF; CENPF and EXO1; CENPF and UBE2T; CENPF and RRM2; CENPF and MLPH; CENPF and GPR160; CENPF and CCNB1; CENPF and CXXC5; CENPF and PTTG1; CENPF and FGFR4; CENPF and FOXC1; CENPF and ESR1; CENPF and ANLN; CENPF and BLVRA; CENPF and EGFR; CENPF and ACTR3B; CENPF and NAT1; CENPF and MYC; CENPF and SFRP1; CENPF and MELK; CENPF and BAG1; CENPF and CEP55; CENPF and MKI67; CENPF and TMEM45B; CENPF and PGR; CENPF and MDM2; CENPF and KRT5; CENPF and FOXA1; CENPF and ORC6; CENPF and CDH3; CENPF and ERBB2; CENPF and GRB7; CENPF and CDC6; CENPF and MAPT; CENPF and BIRC5; CENPF and KRT14; CENPF and KRT17; CENPF and TYMS; CENPF and NDC80; CENPF and SLC39A6; CENPF and BCL2; CENPF and CCNE1; CENPF and MIA; CENPF and MYBL2; CENPF and UBE2C; CENPF and MMP11; CENPF, EXO1 and UBE2T; CENPF, EXO1 and RRM2; CENPF, EXO1 and MLPH; CENPF, EXO1 and GPR160; CENPF, EXO1 and CCNB1; CENPF, EXO1 and CXXC5; CENPF, EXO1 and PTTG1; CENPF, EXO1 and FGFR4; CENPF, EXO1 and FOXC1; CENPF, EXO1 and ESR1; CENPF, EXO1 and ANLN; CENPF, EXO1 and BLVRA; CENPF, EXO1 and EGFR; CENPF, EXO1 and ACTR3B; CENPF, EXO1 and NAT1; CENPF, EXO1 and MYC; CENPF, EXO1 and SFRP1; CENPF, EXO1 and MELK; CENPF, EXO1 and BAG1; CENPF, EXO1 and CEP55; CENPF, EXO1 and MKI67; CENPF, EXO1 and TMEM45B; CENPF, EXO1 and PGR; CENPF, EXO1 and MDM2; CENPF, EXO1 and KRT5; CENPF, EXO1 and FOXA1; CENPF, EXO1 and ORC6; CENPF, EXO1 and CDH3; CENPF, EXO1 and ERBB2; CENPF, EXO1 and GRB7; CENPF, EXO1 and CDC6; CENPF, EXO1 and MAPT; CENPF, EXO1 and BIRC5; CENPF, EXO1 and KRT14; CENPF, EXO1 and KRT17; CENPF, EXO1 and TYMS; CENPF, EXO1 and NDC80; CENPF, EXO1 and SLC39A6; CENPF, EXO1 and BCL2; CENPF, EXO1 and CCNE1; CENPF, EXO1 and MIA; CENPF, EXO1 and MYBL2; CENPF, EXO1 and UBE2C; CENPF, EXO1 and MMP11; CENPF, EXO1, UBE2T and RRM2; CENPF, EXO1, UBE2T and MLPH; CENPF, EXO1, UBE2T and GPR160; CENPF, EXO1, UBE2T and CCNB1; CENPF, EXO1, UBE2T and CXXC5; CENPF, EXO1, UBE2T and PTTG1; CENPF, EXO1, UBE2T and FGFR4; CENPF, EXO1, UBE2T and FOXC1; CENPF, EXO1, UBE2T and ESR1; CENPF, EXO1, UBE2T and ANLN; CENPF, EXO1, UBE2T and BLVRA; CENPF, EXO1, UBE2T and EGFR; CENPF, EXO1, UBE2T and ACTR3B; CENPF, EXO1, UBE2T and NAT1; CENPF, EXO1, UBE2T and MYC; CENPF, EXO1, UBE2T and SFRP1; CENPF, EXO1, UBE2T and MELK; CENPF, EXO1, UBE2T and BAG1; CENPF, EXO1, UBE2T and CEP55; CENPF, EXO1, UBE2T and MKI67; CENPF, EXO1, UBE2T and TMEM45B; CENPF, EXO1, UBE2T and PGR; CENPF, EXO1, UBE2T and MDM2; CENPF, EXO1, UBE2T and KRT5; CENPF, EXO1, UBE2T and FOXA1; CENPF, EXO1, UBE2T and ORC6; CENPF, EXO1, UBE2T and CDH3; CENPF, EXO1, UBE2T and ERBB2; CENPF, EXO1, UBE2T and GRB7; CENPF, EXO1, UBE2T and CDC6; CENPF, EXO1, UBE2T and MAPT; CENPF, EXO1, UBE2T and BIRC5; CENPF, EXO1, UBE2T and KRT14; CENPF, EXO1, UBE2T and KRT17; CENPF, EXO1, UBE2T and TYMS; CENPF, EXO1, UBE2T and NDC80; CENPF, EXO1, UBE2T and SLC39A6; CENPF, EXO1, UBE2T and BCL2; CENPF, EXO1, UBE2T and CCNE1; CENPF, EXO1, UBE2T and MIA; CENPF, EXO1, UBE2T and MYBL2; CENPF, EXO1, UBE2T and UBE2C; CENPF, EXO1, UBE2T and MMP11; EXO1; EXO1 and UBE2T; EXO1 and RRM2; EXO1 and MLPH; EXO1 and GPR160; EXO1 and CCNB1; EXO1 and CXXC5; EXO1 and PTTG1; EXO1 and FGFR4; EXO1 and FOXC1; EXO1 and ESR1; EXO1 and ANLN; EXO1 and BLVRA; EXO1 and EGFR; EXO1 and ACTR3B; EXO1 and NAT1; EXO1 and MYC; EXO1 and SFRP1; EXO1 and MELK; EXO1 and BAG1; EXO1 and CEP55; EXO1 and MKI67; EXO1 and TMEM45B; EXO1 and PGR; EXO1 and MDM2; EXO1 and KRT5; EXO1 and FOXA1; EXO1 and ORC6; EXO1 and CDH3; EXO1 and ERBB2; EXO1 and GRB7; EXO1 and CDC6; EXO1 and MAPT; EXO1 and BIRC5; EXO1 and KRT14; EXO1 and KRT17; EXO1 and TYMS; EXO1 and NDC80; EXO1 and SLC39A6; EXO1 and BCL2; EXO1 and CCNE1; EXO1 and MIA; EXO1 and MYBL2; EXO1 and UBE2C; EXO1 and MMP11; EXO1, UBE2T and RRM2; EXO1, UBE2T and MLPH; EXO1, UBE2T and GPR160; EXO1, UBE2T and CCNB1; EXO1, UBE2T and CXXC5; EXO1, UBE2T and PTTG1; EXO1, UBE2T and FGFR4; EXO1, UBE2T and FOXC1; EXO1, UBE2T and ESR1; EXO1, UBE2T and ANLN; EXO1, UBE2T and BLVRA; EXO1, UBE2T and EGFR; EXO1, UBE2T and ACTR3B; EXO1, UBE2T and NAT1; EXO1, UBE2T and MYC; EXO1, UBE2T and SFRP1; EXO1, UBE2T and MELK; EXO1, UBE2T and BAG1; EXO1, UBE2T and CEP55; EXO1, UBE2T and MKI67; EXO1, UBE2T and TMEM45B; EXO1, UBE2T and PGR; EXO1, UBE2T and MDM2; EXO1, UBE2T and KRT5; EXO1, UBE2T and FOXA1; EXO1, UBE2T and ORC6; EXO1, UBE2T and CDH3; EXO1, UBE2T and ERBB2; EXO1, UBE2T and GRB7; EXO1, UBE2T and CDC6; EXO1, UBE2T and MAPT; EXO1, UBE2T and BIRC5; EXO1, UBE2T and KRT14; EXO1, UBE2T and KRT17; EXO1, UBE2T and TYMS; EXO1, UBE2T and NDC80; EXO1, UBE2T and SLC39A6; EXO1, UBE2T and BCL2; EXO1, UBE2T and CCNE1; EXO1, UBE2T and MIA; EXO1, UBE2T and MYBL2; EXO1, UBE2T and UBE2C; EXO1, UBE2T and MMP11; EXO1, UBE2T, RRM2 and MLPH; EXO1, UBE2T, RRM2 and GPR160; EXO1, UBE2T, RRM2 and CCNB1; EXO1, UBE2T, RRM2 and CXXC5; EXO1, UBE2T, RRM2 and PTTG1; EXO1, UBE2T, RRM2 and FGFR4; EXO1, UBE2T, RRM2 and FOXC1; EXO1, UBE2T, RRM2 and ESR1; EXO1, UBE2T, RRM2 and ANLN; EXO1, UBE2T, RRM2 and BLVRA; EXO1, UBE2T, RRM2 and EGFR; EXO1, UBE2T, RRM2 and ACTR3B; EXO1, UBE2T, RRM2 and NAT1; EXO1, UBE2T, RRM2 and MYC; EXO1, UBE2T, RRM2 and SFRP1; EXO1, UBE2T, RRM2 and MELK; EXO1, UBE2T, RRM2 and BAG1; EXO1, UBE2T, RRM2 and CEP55; EXO1, UBE2T, RRM2 and MKI67; EXO1, UBE2T, RRM2 and TMEM45B; EXO1, UBE2T, RRM2 and PGR; EXO1, UBE2T, RRM2 and MDM2; EXO1, UBE2T, RRM2 and KRT5; EXO1, UBE2T, RRM2 and FOXA1; EXO1, UBE2T, RRM2 and ORC6; EXO1, UBE2T, RRM2 and CDH3; EXO1, UBE2T, RRM2 and ERBB2; EXO1, UBE2T, RRM2 and GRB7; EXO1, UBE2T, RRM2 and CDC6; EXO1, UBE2T, RRM2 and MAPT; EXO1, UBE2T, RRM2 and BIRC5; EXO1, UBE2T, RRM2 and KRT14; EXO1, UBE2T, RRM2 and KRT17; EXO1, UBE2T, RRM2 and TYMS; EXO1, UBE2T, RRM2 and NDC80; EXO1, UBE2T, RRM2 and SLC39A6; EXO1, UBE2T, RRM2 and BCL2; EXO1, UBE2T, RRM2 and CCNE1; EXO1, UBE2T, RRM2 and MIA; EXO1, UBE2T, RRM2 and MYBL2; EXO1, UBE2T, RRM2 and UBE2C; EXO1, UBE2T, RRM2 and MMP11; UBE2T; UBE2T and RRM2; UBE2T and MLPH; UBE2T and GPR160; UBE2T and CCNB1; UBE2T and CXXC5; UBE2T and PTTG1; UBE2T and FGFR4; UBE2T and FOXC1; UBE2T and ESR1; UBE2T and ANLN; UBE2T and BLVRA; UBE2T and EGFR; UBE2T and ACTR3B; UBE2T and NAT1; UBE2T and MYC; UBE2T and SFRP1; UBE2T and MELK; UBE2T and BAG1; UBE2T and CEP55; UBE2T and MKI67; UBE2T and TMEM45B; UBE2T and PGR; UBE2T and MDM2; UBE2T and KRT5; UBE2T and FOXA1; UBE2T and ORC6; UBE2T and CDH3; UBE2T and ERBB2; UBE2T and GRB7; UBE2T and CDC6; UBE2T and MAPT; UBE2T and BIRC5; UBE2T and KRT14; UBE2T and KRT17; UBE2T and TYMS; UBE2T and NDC80; UBE2T and SLC39A6; UBE2T and BCL2; UBE2T and CCNE1; UBE2T and MIA; UBE2T and MYBL2; UBE2T and UBE2C; UBE2T and MMP11; RRM2; RRM2 and MLPH; RRM2 and GPR160; RRM2 and CCNB1; RRM2 and CXXC5; RRM2 and PTTG1; RRM2 and FGFR4; RRM2 and FOXC1; RRM2 and ESR1; RRM2 and ANLN; RRM2 and BLVRA; RRM2 and EGFR; RRM2 and ACTR3B; RRM2 and NAT1; RRM2 and MYC; RRM2 and SFRP1; RRM2 and MELK; RRM2 and BAG1; RRM2 and CEP55; RRM2 and MKI67; RRM2 and TMEM45B; RRM2 and PGR; RRM2 and MDM2; RRM2 and KRT5; RRM2 and FOXA1; RRM2 and ORC6; RRM2 and CDH3; RRM2 and ERBB2; RRM2 and GRB7; RRM2 and CDC6; RRM2 and MAPT; RRM2 and BIRC5; RRM2 and KRT14; RRM2 and KRT17; RRM2 and TYMS; RRM2 and NDC80; RRM2 and SLC39A6; RRM2 and BCL2; RRM2 and CCNE1; RRM2 and MIA; RRM2 and MYBL2; RRM2 and UBE2C; RRM2 and MMP11; MLPH; MLPH and GPR160; MLPH and CCNB1; MLPH and CXXC5; MLPH and PTTG1; MLPH and FGFR4; MLPH and FOXC1; MLPH and ESR1; MLPH and ANLN; MLPH and BLVRA; MLPH and EGFR; MLPH and ACTR3B; MLPH and NAT1; MLPH and MYC; MLPH and SFRP1; MLPH and MELK; MLPH and BAG1; MLPH and CEP55; MLPH and MKI67; MLPH and TMEM45B; MLPH and PGR; MLPH and MDM2; MLPH and KRT5; MLPH and FOXA1; MLPH and ORC6; MLPH and CDH3; MLPH and ERBB2; MLPH and GRB7; MLPH and CDC6; MLPH and MAPT; MLPH and BIRC5; MLPH and KRT14; MLPH and KRT17; MLPH and TYMS; MLPH and NDC80; MLPH and SLC39A6; MLPH and BCL2; MLPH and CCNE1; MLPH and MIA; MLPH and MYBL2; MLPH and UBE2C; MLPH and MMP11; GPR160; GPR160 and CCNB1; GPR160 and CXXC5; GPR160 and PTTG1; GPR160 and FGFR4; GPR160 and FOXC1; GPR160 and ESR1; GPR160 and ANLN; GPR160 and BLVRA; GPR160 and EGFR; GPR160 and ACTR3B; GPR160 and NAT1; GPR160 and MYC; GPR160 and SFRP1; GPR160 and MELK; GPR160 and BAG1; GPR160 and CEP55; GPR160 and MKI67; GPR160 and TMEM45B; GPR160 and PGR; GPR160 and MDM2; GPR160 and KRT5; GPR160 and FOXA1; GPR160 and ORC6; GPR160 and CDH3; GPR160 and ERBB2; GPR160 and GRB7; GPR160 and CDC6; GPR160 and MAPT; GPR160 and BIRC5; GPR160 and KRT14; GPR160 and KRT17; GPR160 and TYMS; GPR160 and NDC80; GPR160 and SLC39A6; GPR160 and BCL2; GPR160 and CCNE1; GPR160 and MIA; GPR160 and MYBL2; GPR160 and UBE2C; GPR160 and MMP11; CCNB1; CCNB1 and CXXC5; CCNB1 and PTTG1; CCNB1 and FGFR4; CCNB1 and FOXC1; CCNB1 and ESR1; CCNB1 and ANLN; CCNB1 and BLVRA; CCNB1 and EGFR; CCNB1 and ACTR3B; CCNB1 and NAT1; CCNB1 and MYC; CCNB1 and SFRP1; CCNB1 and MELK; CCNB1 and BAG1; CCNB1 and CEP55; CCNB1 and MKI67; CCNB1 and TMEM45B; CCNB1 and PGR; CCNB1 and MDM2; CCNB1 and KRT5; CCNB1 and FOXA1; CCNB1 and ORC6; CCNB1 and CDH3; CCNB1 and ERBB2; CCNB1 and GRB7; CCNB1 and CDC6; CCNB1 and MAPT; CCNB1 and BIRC5; CCNB1 and KRT14; CCNB1 and KRT17; CCNB1 and TYMS; CCNB1 and NDC80; CCNB1 and SLC39A6; CCNB1 and BCL2; CCNB1 and CCNE1; CCNB1 and MIA; CCNB1 and MYBL2; CCNB1 and UBE2C; CCNB1 and MMP11; CXXC5; CXXC5 and PTTG1; CXXC5 and FGFR4; CXXC5 and FOXC1; CXXC5 and ESR1; CXXC5 and ANLN; CXXC5 and BLVRA; CXXC5 and EGFR; CXXC5 and ACTR3B; CXXC5 and NAT1; CXXC5 and MYC; CXXC5 and SFRP1; CXXC5 and MELK; CXXC5 and BAG1; CXXC5 and CEP55; CXXC5 and MKI67; CXXC5 and TMEM45B; CXXC5 and PGR; CXXC5 and MDM2; CXXC5 and KRT5; CXXC5 and FOXA1; CXXC5 and ORC6; CXXC5 and CDH3; CXXC5 and ERBB2; CXXC5 and GRB7; CXXC5 and CDC6; CXXC5 and MAPT; CXXC5 and BIRC5; CXXC5 and KRT14; CXXC5 and KRT17; CXXC5 and TYMS; CXXC5 and NDC80; CXXC5 and SLC39A6; CXXC5 and BCL2; CXXC5 and CCNE1; CXXC5 and MIA; CXXC5 and MYBL2; CXXC5 and UBE2C; CXXC5 and MMP11; PTTG1; PTTG1 and FGFR4; PTTG1 and FOXC1; PTTG1 and ESR1; PTTG1 and ANLN; PTTG1 and BLVRA; PTTG1 and EGFR; PTTG1 and ACTR3B; PTTG1 and NAT1; PTTG1 and MYC; PTTG1 and SFRP1; PTTG1 and MELK; PTTG1 and BAG1; PTTG1 and CEP55; PTTG1 and MKI67; PTTG1 and TMEM45B; PTTG1 and PGR; PTTG1 and MDM2; PTTG1 and KRT5; PTTG1 and FOXA1; PTTG1 and ORC6; PTTG1 and CDH3; PTTG1 and ERBB2; PTTG1 and GRB7; PTTG1 and CDC6; PTTG1 and MAPT; PTTG1 and BIRC5; PTTG1 and KRT14; PTTG1 and KRT17; PTTG1 and TYMS; PTTG1 and NDC80; PTTG1 and SLC39A6; PTTG1 and BCL2; PTTG1 and CCNE1; PTTG1 and MIA; PTTG1 and MYBL2; PTTG1 and UBE2C; PTTG1 and MMP11; FGFR4; FGFR4 and FOXC1; FGFR4 and ESR1; FGFR4 and ANLN; FGFR4 and BLVRA; FGFR4 and EGFR; FGFR4 and ACTR3B; FGFR4 and NAT1; FGFR4 and MYC; FGFR4 and SFRP1; FGFR4 and MELK; FGFR4 and BAG1; FGFR4 and CEP55; FGFR4 and MKI67; FGFR4 and TMEM45B; FGFR4 and PGR; FGFR4 and MDM2; FGFR4 and KRT5; FGFR4 and FOXA1; FGFR4 and ORC6; FGFR4 and CDH3; FGFR4 and ERBB2; FGFR4 and GRB7; FGFR4 and CDC6; FGFR4 and MAPT; FGFR4 and BIRC5; FGFR4 and KRT14; FGFR4 and KRT17; FGFR4 and TYMS; FGFR4 and NDC80; FGFR4 and SLC39A6; FGFR4 and BCL2; FGFR4 and CCNE1; FGFR4 and MIA; FGFR4 and MYBL2; FGFR4 and UBE2C; FGFR4 and MMP11; FOXC1; FOXC1 and ESR1; FOXC1 and ANLN; FOXC1 and BLVRA; FOXC1 and EGFR; FOXC1 and ACTR3B; FOXC1 and NAT1; FOXC1 and MYC; FOXC1 and SFRP1; FOXC1 and MELK; FOXC1 and BAG1; FOXC1 and CEP55; FOXC1 and MKI67; FOXC1 and TMEM45B; FOXC1 and PGR; FOXC1 and MDM2; FOXC1 and KRT5; FOXC1 and FOXA1; FOXC1 and ORC6; FOXC1 and CDH3; FOXC1 and ERBB2; FOXC1 and GRB7; FOXC1 and CDC6; FOXC1 and MAPT; FOXC1 and BIRC5; FOXC1 and KRT14; FOXC1 and KRT17; FOXC1 and TYMS; FOXC1 and NDC80; FOXC1 and SLC39A6; FOXC1 and BCL2; FOXC1 and CCNE1; FOXC1 and MIA; FOXC1 and MYBL2; FOXC1 and UBE2C; FOXC1 and MMP11; ESR1; ESR1 and ANLN; ESR1 and BLVRA; ESR1 and EGFR; ESR1 and ACTR3B; ESR1 and NAT1; ESR1 and MYC; ESR1 and SFRP1; ESR1 and MELK; ESR1 and BAG1; ESR1 and CEP55; ESR1 and MKI67; ESR1 and TMEM45B; ESR1 and PGR; ESR1 and MDM2; ESR1 and KRT5; ESR1 and FOXA1; ESR1 and ORC6; ESR1 and CDH3; ESR1 and ERBB2; ESR1 and GRB7; ESR1 and CDC6; ESR1 and MAPT; ESR1 and BIRC5; ESR1 and KRT14; ESR1 and KRT17; ESR1 and TYMS; ESR1 and NDC80; ESR1 and SLC39A6; ESR1 and BCL2; ESR1 and CCNE1; ESR1 and MIA; ESR1 and MYBL2; ESR1 and UBE2C; ESR1 and MMP11; ANLN; ANLN and BLVRA; ANLN and EGFR; ANLN and ACTR3B; ANLN and NAT1; ANLN and MYC; ANLN and SFRP1; ANLN and MELK; ANLN and BAG1; ANLN and CEP55; ANLN and MKI67; ANLN and TMEM45B; ANLN and PGR; ANLN and MDM2; ANLN and KRT5; ANLN and FOXA1; ANLN and ORC6; ANLN and CDH3; ANLN and ERBB2; ANLN and GRB7; ANLN and CDC6; ANLN and MAPT; ANLN and BIRC5; ANLN and KRT14; ANLN and KRT17; ANLN and TYMS; ANLN and NDC80; ANLN and SLC39A6; ANLN and BCL2; ANLN and CCNE1; ANLN and MIA; ANLN and MYBL2; ANLN and UBE2C; ANLN and MMP11; BLVRA; BLVRA and EGFR; BLVRA and ACTR3B; BLVRA and NAT1; BLVRA and MYC; BLVRA and SFRP1; BLVRA and MELK; BLVRA and BAG1; BLVRA and CEP55; BLVRA and MKI67; BLVRA and TMEM45B; BLVRA and PGR; BLVRA and MDM2; BLVRA and KRT5; BLVRA and FOXA1; BLVRA and ORC6; BLVRA and CDH3; BLVRA and ERBB2; BLVRA and GRB7; BLVRA and CDC6; BLVRA and MAPT; BLVRA and BIRC5; BLVRA and KRT14; BLVRA and KRT17; BLVRA and TYMS; BLVRA and NDC80; BLVRA and SLC39A6; BLVRA and BCL2; BLVRA and CCNE1; BLVRA and MIA; BLVRA and MYBL2; BLVRA and UBE2C; BLVRA and MMP11; EGFR; EGFR and ACTR3B; EGFR and NAT1; EGFR and MYC; EGFR and SFRP1; EGFR and MELK; EGFR and BAG1; EGFR and CEP55; EGFR and MKI67; EGFR and TMEM45B; EGFR and PGR; EGFR and MDM2; EGFR and KRT5; EGFR and FOXA1; EGFR and ORC6; EGFR and CDH3; EGFR and ERBB2; EGFR and GRB7; EGFR and CDC6; EGFR and MAPT; EGFR and BIRC5; EGFR and KRT14; EGFR and KRT17; EGFR and TYMS; EGFR and NDC80; EGFR and SLC39A6; EGFR and BCL2; EGFR and CCNE1; EGFR and MIA; EGFR and MYBL2; EGFR and UBE2C; EGFR and MMP11; ACTR3B; ACTR3B and NAT1; ACTR3B and MYC; ACTR3B and SFRP1; ACTR3B and MELK; ACTR3B and BAG1; ACTR3B and CEP55; ACTR3B and MKI67; ACTR3B and TMEM45B; ACTR3B and PGR; ACTR3B and MDM2; ACTR3B and KRT5; ACTR3B and FOXA1; ACTR3B and ORC6; ACTR3B and CDH3; ACTR3B and ERBB2; ACTR3B and GRB7; ACTR3B and CDC6; ACTR3B and MAPT; ACTR3B and BIRC5; ACTR3B and KRT14; ACTR3B and KRT17; ACTR3B and TYMS; ACTR3B and NDC80; ACTR3B and SLC39A6; ACTR3B and BCL2; ACTR3B and CCNE1; ACTR3B and MIA; ACTR3B and MYBL2; ACTR3B and UBE2C; ACTR3B and MMP11; NAT1; NAT1 and MYC; NAT1 and SFRP1; NAT1 and MELK; NAT1 and BAG1; NAT1 and CEP55; NAT1 and MKI67; NAT1 and TMEM45B; NAT1 and PGR; NAT1 and MDM2; NAT1 and KRT5; NAT1 and FOXA1; NAT1 and ORC6; NAT1 and CDH3; NAT1 and ERBB2; NAT1 and GRB7; NAT1 and CDC6; NAT1 and MAPT; NAT1 and BIRC5; NAT1 and KRT14; NAT1 and KRT17; NAT1 and TYMS; NAT1 and NDC80; NAT1 and SLC39A6; NAT1 and BCL2; NAT1 and CCNE1; NAT1 and MIA; NAT1 and MYBL2; NAT1 and UBE2C; NAT1 and MMP11; MYC; MYC and SFRP1; MYC and MELK; MYC and BAG1; MYC and CEP55; MYC and MKI67; MYC and TMEM45B; MYC and PGR; MYC and MDM2; MYC and KRT5; MYC and FOXA1; MYC and ORC6; MYC and CDH3; MYC and ERBB2; MYC and GRB7; MYC and CDC6; MYC and MAPT; MYC and BIRC5; MYC and KRT14; MYC and KRT17; MYC and TYMS; MYC and NDC80; MYC and SLC39A6; MYC and BCL2; MYC and CCNE1; MYC and MIA; MYC and MYBL2; MYC and UBE2C; MYC and MMP11; SFRP1; SFRP1 and MELK; SFRP1 and BAG1; SFRP1 and CEP55; SFRP1 and MKI67; SFRP1 and TMEM45B; SFRP1 and PGR; SFRP1 and MDM2; SFRP1 and KRT5; SFRP1 and FOXA1; SFRP1 and ORC6; SFRP1 and CDH3; SFRP1 and ERBB2; SFRP1 and GRB7; SFRP1 and CDC6; SFRP1 and MAPT; SFRP1 and BIRC5; SFRP1 and KRT14; SFRP1 and KRT17; SFRP1 and TYMS; SFRP1 and NDC80; SFRP1 and SLC39A6; SFRP1 and BCL2; SFRP1 and CCNE1; SFRP1 and MIA; SFRP1 and MYBL2; SFRP1 and UBE2C; SFRP1 and MMP11; MELK; MELK and BAG1; MELK and CEP55; MELK and MKI67; MELK and TMEM45B; MELK and PGR; MELK and MDM2; MELK and KRT5; MELK and FOXA1; MELK and ORC6; MELK and CDH3; MELK and ERBB2; MELK and GRB7; MELK and CDC6; MELK and MAPT; MELK and BIRC5; MELK and KRT14; MELK and KRT17; MELK and TYMS; MELK and NDC80; MELK and SLC39A6; MELK and BCL2; MELK and CCNE1; MELK and MIA; MELK and MYBL2; MELK and UBE2C; MELK and MMP11; BAG1; BAG1 and CEP55; BAG1 and MKI67; BAG1 and TMEM45B; BAG1 and PGR; BAG1 and MDM2; BAG1 and KRT5; BAG1 and FOXA1; BAG1 and ORC6; BAG1 and CDH3; BAG1 and ERBB2; BAG1 and GRB7; BAG1 and CDC6; BAG1 and MAPT; BAG1 and BIRC5; BAG1 and KRT14; BAG1 and KRT17; BAG1 and TYMS; BAG1 and NDC80; BAG1 and SLC39A6; BAG1 and BCL2; BAG1 and CCNE1; BAG1 and MIA; BAG1 and MYBL2; BAG1 and UBE2C; BAG1 and MMP11; CEP55; CEP55 and MKI67; CEP55 and TMEM45B; CEP55 and PGR; CEP55 and MDM2; CEP55 and KRT5; CEP55 and FOXA1; CEP55 and ORC6; CEP55 and CDH3; CEP55 and ERBB2; CEP55 and GRB7; CEP55 and CDC6; CEP55 and MAPT; CEP55 and BIRC5; CEP55 and KRT14; CEP55 and KRT17; CEP55 and TYMS; CEP55 and NDC80; CEP55 and SLC39A6; CEP55 and BCL2; CEP55 and CCNE1; CEP55 and MIA; CEP55 and MYBL2; CEP55 and UBE2C; CEP55 and MMP11; MKI67; MKI67 and TMEM45B; MKI67 and PGR; MKI67 and MDM2; MKI67 and KRT5; MKI67 and FOXA1; MKI67 and ORC6; MKI67 and CDH3; MKI67 and ERBB2; MKI67 and GRB7; MKI67 and CDC6; MKI67 and MAPT; MKI67 and BIRC5; MKI67 and KRT14; MKI67 and KRT17; MKI67 and TYMS; MKI67 and NDC80; MKI67 and SLC39A6; MKI67 and BCL2; MKI67 and CCNE1; MKI67 and MIA; MKI67 and MYBL2; MKI67 and UBE2C; MKI67 and MMP11; TMEM45B; TMEM45B and PGR; TMEM45B and MDM2; TMEM45B and KRT5; TMEM45B and FOXA1; TMEM45B and ORC6; TMEM45B and CDH3; TMEM45B and ERBB2; TMEM45B and GRB7; TMEM45B and CDC6; TMEM45B and MAPT; TMEM45B and BIRC5; TMEM45B and KRT14; TMEM45B and KRT17; TMEM45B and TYMS; TMEM45B and NDC80; TMEM45B and SLC39A6; TMEM45B and BCL2; TMEM45B and CCNE1; TMEM45B and MIA; TMEM45B and MYBL2; TMEM45B and UBE2C; TMEM45B and MMP11; PGR; PGR and MDM2; PGR and KRT5; PGR and FOXA1; PGR and ORC6; PGR and CDH3; PGR and ERBB2; PGR and GRB7; PGR and CDC6; PGR and MAPT; PGR and BIRC5; PGR and KRT14; PGR and KRT17; PGR and TYMS; PGR and NDC80; PGR and SLC39A6; PGR and BCL2; PGR and CCNE1; PGR and MIA; PGR and MYBL2; PGR and UBE2C; PGR and MMP11; MDM2; MDM2 and KRT5; MDM2 and FOXA1; MDM2 and ORC6; MDM2 and CDH3; MDM2 and ERBB2; MDM2 and GRB7; MDM2 and CDC6; MDM2 and MAPT; MDM2 and BIRC5; MDM2 and KRT14; MDM2 and KRT17; MDM2 and TYMS; MDM2 and NDC80; MDM2 and SLC39A6; MDM2 and BCL2; MDM2 and CCNE1; MDM2 and MIA; MDM2 and MYBL2; MDM2 and UBE2C; MDM2 and MMP11; KRT5; KRT5 and FOXA1; KRT5 and ORC6; KRT5 and CDH3; KRT5 and ERBB2; KRT5 and GRB7; KRT5 and CDC6; KRT5 and MAPT; KRT5 and BIRC5; KRT5 and KRT14; KRT5 and KRT17; KRT5 and TYMS; KRT5 and NDC80; KRT5 and SLC39A6; KRT5 and BCL2; KRT5 and CCNE1; KRT5 and MIA; KRT5 and MYBL2; KRT5 and UBE2C; KRT5 and MMP11; FOXA1; FOXA1 and ORC6; FOXA1 and CDH3; FOXA1 and ERBB2; FOXA1 and GRB7; FOXA1 and CDC6; FOXA1 and MAPT; FOXA1 and BIRC5; FOXA1 and KRT14; FOXA1 and KRT17; FOXA1 and TYMS; FOXA1 and NDC80; FOXA1 and SLC39A6; FOXA1 and BCL2; FOXA1 and CCNE1; FOXA1 and MIA; FOXA1 and MYBL2; FOXA1 and UBE2C; FOXA1 and MMP11; ORC6; ORC6 and CDH3; ORC6 and ERBB2; ORC6 and GRB7; ORC6 and CDC6; ORC6 and MAPT; ORC6 and BIRC5; ORC6 and KRT14; ORC6 and KRT17; ORC6 and TYMS; ORC6 and NDC80; ORC6 and SLC39A6; ORC6 and BCL2; ORC6 and CCNE1; ORC6 and MIA; ORC6 and MYBL2; ORC6 and UBE2C; ORC6 and MMP11; CDH3; CDH3 and ERBB2; CDH3 and GRB7; CDH3 and CDC6; CDH3 and MAPT; CDH3 and BIRC5; CDH3 and KRT14; CDH3 and KRT17; CDH3 and TYMS; CDH3 and NDC80; CDH3 and SLC39A6; CDH3 and BCL2; CDH3 and CCNE1; CDH3 and MIA; CDH3 and MYBL2; CDH3 and UBE2C; CDH3 and MMP11; ERBB2; ERBB2 and GRB7; ERBB2 and CDC6; ERBB2 and MAPT; ERBB2 and BIRC5; ERBB2 and KRT14; ERBB2 and KRT17; ERBB2 and TYMS; ERBB2 and NDC80; ERBB2 and SLC39A6; ERBB2 and BCL2; ERBB2 and CCNE1; ERBB2 and MIA; ERBB2 and MYBL2; ERBB2 and UBE2C; ERBB2 and MMP11; GRB7; GRB7 and CDC6; GRB7 and MAPT; GRB7 and BIRC5; GRB7 and KRT14; GRB7 and KRT17; GRB7 and TYMS; GRB7 and NDC80; GRB7 and SLC39A6; GRB7 and BCL2; GRB7 and CCNE1; GRB7 and MIA; GRB7 and MYBL2; GRB7 and UBE2C; GRB7 and MMP11; CDC6; CDC6 and MAPT; CDC6 and BIRC5; CDC6 and KRT14; CDC6 and KRT17; CDC6 and TYMS; CDC6 and NDC80; CDC6 and SLC39A6; CDC6 and BCL2; CDC6 and CCNE1; CDC6 and MIA; CDC6 and MYBL2; CDC6 and UBE2C; CDC6 and MMP11; MAPT; MAPT and BIRC5; MAPT and KRT14; MAPT and KRT17; MAPT and TYMS; MAPT and NDC80; MAPT and SLC39A6; MAPT and BCL2; MAPT and CCNE1; MAPT and MIA; MAPT and MYBL2; MAPT and UBE2C; MAPT and MMP11; BIRC5; BIRC5 and KRT14; BIRC5 and KRT17; BIRC5 and TYMS; BIRC5 and NDC80; BIRC5 and SLC39A6; BIRC5 and BCL2; BIRC5 and CCNE1; BIRC5 and MIA; BIRC5 and MYBL2; BIRC5 and UBE2C; BIRC5 and MMP11; KRT14; KRT14 and KRT17; KRT14 and TYMS; KRT14 and NDC80; KRT14 and SLC39A6; KRT14 and BCL2; KRT14 and CCNE1; KRT14 and MIA; KRT14 and MYBL2; KRT14 and UBE2C; KRT14 and MMP11; KRT17; KRT17 and TYMS; KRT17 and NDC80; KRT17 and SLC39A6; KRT17 and BCL2; KRT17 and CCNE1; KRT17 and MIA; KRT17 and MYBL2; KRT17 and UBE2C; KRT17 and MMP11; TYMS; TYMS and NDC80; TYMS and SLC39A6; TYMS and BCL2; TYMS and CCNE1; TYMS and MIA; TYMS and MYBL2; TYMS and UBE2C; TYMS and MMP11; NDC80; NDC80 and SLC39A6; NDC80 and BCL2; NDC80 and CCNE1; NDC80 and MIA; NDC80 and MYBL2; NDC80 and UBE2C; NDC80 and MMP11; SLC39A6; SLC39A6 and BCL2; SLC39A6 and CCNE1; SLC39A6 and MIA; SLC39A6 and MYBL2; SLC39A6 and UBE2C; SLC39A6 and MMP11; BCL2; BCL2 and CCNE1; BCL2 and MIA; BCL2 and MYBL2; BCL2 and UBE2C; BCL2 and MMP11; CCNE1; CCNE1 and MIA; CCNE1 and MYBL2; CCNE1 and UBE2C; CCNE1 and MMP11; MIA; MIA and MYBL2; MIA and UBE2C; MIA and MMP11; MYBL2; MYBL2 and UBE2C; MYBL2 and MMP11; UBE2C; UBE2C and MMP11; MMP11; TDRD1, CACNA1D, NCALD, HLA-DMB, KCNH8, PDE3B, PLA2G7, CSGALNACT1, PART1, HES1, F3, GPR110, SH3RF, PDE8B, and SEPT9; TDRD1; TDRD1 and CACNA1D; TDRD1 and NCALD; TDRD1 and HLA-DMB; TDRD1 and KCNH8; TDRD1 and PDE3B; TDRD1 and PLA2G7; TDRD1 and CSGALNACT1; TDRD1 and PART1; TDRD1 and HES1; TDRD1 and F3; TDRD1 and GPR110; TDRD1 and SH3RF; TDRD1 and PDE8B; TDRD1 and SEPT9; CACNA1D; CACNA1D and NCALD; CACNA1D and HLA-DMB; CACNA1D and KCNH8; CACNA1D and PDE3B; CACNA1D and PLA2G7; CACNA1D and CSGALNACT1; CACNA1D and PART1; CACNA1D and HES1; CACNA1D and F3; CACNA1D and GPR110; CACNA1D and SH3RF; CACNA1D and PDE8B; CACNA1D and SEPT9; NCALD; NCALD and HLA-DMB; NCALD and KCNH8; NCALD and PDE3B; NCALD and PLA2G7; NCALD and CSGALNACT1; NCALD and PART1; NCALD and HES1; NCALD and F3; NCALD and GPR110; NCALD and SH3RF; NCALD and PDE8B; NCALD and SEPT9; HLA-DMB; HLA-DMB and KCNH8; HLA-DMB and PDE3B; HLA-DMB and PLA2G7; HLA-DMB and CSGALNACT1; HLA-DMB and PART1; HLA-DMB and HES1; HLA-DMB and F3; HLA-DMB and GPR110; HLA-DMB and SH3RF; HLA-DMB and PDE8B; HLA-DMB and SEPT9; KCNH8; KCNH8 and PDE3B; KCNH8 and PLA2G7; KCNH8 and CSGALNACT1; KCNH8 and PART1; KCNH8 and HES1; KCNH8 and F3; KCNH8 and GPR110; KCNH8 and SH3RF; KCNH8 and PDE8B; KCNH8 and SEPT9; PDE3B; PDE3B and PLA2G7; PDE3B and CSGALNACT1; PDE3B and PART1; PDE3B and HES1; PDE3B and F3; PDE3B and GPR110; PDE3B and SH3RF; PDE3B and PDE8B; PDE3B and SEPT9; PLA2G7; PLA2G7 and CSGALNACT1; PLA2G7 and PART1; PLA2G7 and HES1; PLA2G7 and F3; PLA2G7 and GPR110; PLA2G7 and SH3RF; PLA2G7 and PDE8B; PLA2G7 and SEPT9; CSGALNACT1; CSGALNACT1 and PART1; CSGALNACT1 and HES1; CSGALNACT1 and F3; CSGALNACT1 and GPR110; CSGALNACT1 and SH3RF; CSGALNACT1 and PDE8B; CSGALNACT1 and SEPT9; PART1; PART1 and HES1; PART1 and F3; PART1 and GPR110; PART1 and SH3RF; PART1 and PDE8B; PART1 and SEPT9; HES1; HES1 and F3; HES1 and GPR110; HES1 and SH3RF; HES1 and PDE8B; HES1 and SEPT9; F3; F3 and GPR110; F3 and SH3RF; F3 and PDE8B; F3 and SEPT9; GPR110; GPR110 and SH3RF; GPR110 and PDE8B; GPR110 and SEPT9; SH3RF; SH3RF and PDE8B; SH3RF and SEPT9; PDE8B; PDE8B and SEPT9; SEPT9; CRISP3, AMD1, KCNG3, PLA1A, MYO6, FRK, GPR110, SH3YL1, ACER3, C8orf4, GHR, ITPR1, KHDRBS3, NPY, and GUCY1A3; CRISP3; CRISP3 and AMD1; CRISP3 and KCNG3; CRISP3 and PLA1A; CRISP3 and MYO6; CRISP3 and FRK; CRISP3 and GPR110; CRISP3 and SH3YL1; CRISP3 and ACER3; CRISP3 and C8orf4; CRISP3 and GHR; CRISP3 and ITPR1; CRISP3 and KHDRBS3; CRISP3 and NPY; CRISP3 and GUCY1A3; AMD1; AMD1 and KCNG3; AMD1 and PLA1A; AMD1 and MYO6; AMD1 and FRK; AMD1 and GPR110; AMD1 and SH3YL1; AMD1 and ACER3; AMD1 and C8orf4; AMD1 and GHR; AMD1 and ITPR1; AMD1 and KHDRBS3; AMD1 and NPY; AMD1 and GUCY1A3; KCNG3; KCNG3 and PLA1A; KCNG3 and MYO6; KCNG3 and FRK; KCNG3 and GPR110; KCNG3 and SH3YL1; KCNG3 and ACER3; KCNG3 and C8orf4; KCNG3 and GHR; KCNG3 and ITPR1; KCNG3 and KHDRBS3; KCNG3 and NPY; KCNG3 and GUCY1A3; PLA1A; PLA1A and MYO6; PLA1A and FRK; PLA1A and GPR110; PLA1A and SH3YL1; PLA1A and ACER3; PLA1A and C8orf4; PLA1A and GHR; PLA1A and ITPR1; PLA1A and KHDRBS3; PLA1A and NPY; PLA1A and GUCY1A3; MYO6; MYO6 and FRK; MYO6 and GPR110; MYO6 and SH3YL1; MYO6 and ACER3; MYO6 and C8orf4; MYO6 and GHR; MYO6 and ITPR1; MYO6 and KHDRBS3; MYO6 and NPY; MYO6 and GUCY1A3; FRK; FRK and GPR110; FRK and SH3YL1; FRK and ACER3; FRK and C8orf4; FRK and GHR; FRK and ITPR1; FRK and KHDRBS3; FRK and NPY; FRK and GUCY1A3; GPR110 and SH3YL1; GPR110 and ACER3; GPR110 and C8orf4; GPR110 and GHR; GPR110 and ITPR1; GPR110 and KHDRBS3; GPR110 and NPY; GPR110 and GUCY1A3; SH3YL1; SH3YL1 and ACER3; SH3YL1 and C8orf4; SH3YL1 and GHR; SH3YL1 and ITPR1; SH3YL1 and KHDRBS3; SH3YL1 and NPY; SH3YL1 and GUCY1A3; ACER3; ACER3 and C8orf4; ACER3 and GHR; ACER3 and ITPR1; ACER3 and KHDRBS3; ACER3 and NPY; ACER3 and GUCY1A3; C8orf4; C8orf4 and GHR; C8orf4 and ITPR1; C8orf4 and KHDRBS3; C8orf4 and NPY; C8orf4 and GUCY1A3; GHR; GHR and ITPR1; GHR and KHDRBS3; GHR and NPY; GHR and GUCY1A3; ITPR1; ITPR1 and KHDRBS3; ITPR1 and NPY; ITPR1 and GUCY1A3; KHDRBS3; KHDRBS3 and NPY; KHDRBS3 and GUCY1A3; NPY; NPY and GUCY1A3; GUCY1A3; ARHGDIB, LAMC2, VWA2, ZNF432, MORN1, CYorf15B, AMPD3, QDPR, HDAC1, KIF16B, GJB1, ITPR3, ZNF615, ANKRD6, and APOD; ARHGDIB; ARHGDIB and LAMC2; ARHGDIB and VWA2; ARHGDIB and ZNF432; ARHGDIB and MORN1; ARHGDIB and CYorf15B; ARHGDIB and AMPD3; ARHGDIB and QDPR; ARHGDIB and HDAC1; ARHGDIB and KIF16B; ARHGDIB and GJB1; ARHGDIB and ITPR3; ARHGDIB and ZNF615; ARHGDIB and ANKRD6; ARHGDIB and APOD; LAMC2; LAMC2 and VWA2; LAMC2 and ZNF432; LAMC2 and MORN1; LAMC2 and CYorf15B; LAMC2 and AMPD3; LAMC2 and QDPR; LAMC2 and HDAC1; LAMC2 and KIF16B; LAMC2 and GJB1; LAMC2 and ITPR3; LAMC2 and ZNF615; LAMC2 and ANKRD6; LAMC2 and APOD; VWA2; VWA2 and ZNF432; VWA2 and MORN1; VWA2 and CYorf15B; VWA2 and AMPD3; VWA2 and QDPR; VWA2 and HDAC1; VWA2 and KIF16B; VWA2 and GJB1; VWA2 and ITPR3; VWA2 and ZNF615; VWA2 and ANKRD6; VWA2 and APOD; ZNF432; ZNF432 and MORN1; ZNF432 and CYorf15B; ZNF432 and AMPD3; ZNF432 and QDPR; ZNF432 and HDAC1; ZNF432 and KIF16B; ZNF432 and GJB1; ZNF432 and ITPR3; ZNF432 and ZNF615; ZNF432 and ANKRD6; ZNF432 and APOD; MORN1; MORN1 and CYorf15B; MORN1 and AMPD3; MORN1 and QDPR; MORN1 and HDAC1; MORN1 and KIF16B; MORN1 and GJB1; MORN1 and ITPR3; MORN1 and ZNF615; MORN1 and ANKRD6; MORN1 and APOD; CYorf15B; CYorf15B and AMPD3; CYorf15B and QDPR; CYorf15B and HDAC1; CYorf15B and KIF16B; CYorf15B and GJB1; CYorf15B and ITPR3; CYorf15B and ZNF615; CYorf15B and ANKRD6; CYorf15B and APOD; AMPD3; AMPD3 and QDPR; AMPD3 and HDAC1; AMPD3 and KIF16B; AMPD3 and GJB1; AMPD3 and ITPR3; AMPD3 and ZNF615; AMPD3 and ANKRD6; AMPD3 and APOD; QDPR; QDPR and HDAC1; QDPR and KIF16B; QDPR and GJB1; QDPR and ITPR3; QDPR and ZNF615; QDPR and ANKRD6; QDPR and APOD; HDAC1; HDAC1 and KIF16B; HDAC1 and GJB1; HDAC1 and ITPR3; HDAC1 and ZNF615; HDAC1 and ANKRD6; HDAC1 and APOD; KIF16B; KIF16B and GJB1; KIF16B and ITPR3; KIF16B and ZNF615; KIF16B and ANKRD6; KIF16B and APOD; GJB1; GJB1 and ITPR3; GJB1 and ZNF615; GJB1 and ANKRD6; GJB1 and APOD; ITPR3; ITPR3 and ZNF615; ITPR3 and ANKRD6; ITPR3 and APOD; ZNF615; ZNF615 and ANKRD6; ZNF615 and APOD; ANKRD6; ANKRD6 and APOD; APOD; STEAP4, RGS17, MAP7, C22orf36, NKAIN1, CHN2, LRRFIP1, SERGEF, ATP8A2, NDRG1, CDC42SE1, LUZP2, HNF1B, TFAP2A and ANKRD34B STEAP4; STEAP4 and RGS17; STEAP4 and MAP7; STEAP4 and C22orf36; STEAP4 and NKAIN1; STEAP4 and CHN2; STEAP4 and LRRFIP1; STEAP4 and SERGEF; STEAP4 and ATP8A2; STEAP4 and NDRG1; STEAP4 and CDC42SE1; STEAP4 and LUZP2; STEAP4 and HNF1B; STEAP4 and TFAP2A; STEAP4 and ANKRD34B; RGS17; RGS17 and MAP7; RGS17 and C22orf36; RGS17 and NKAIN1; RGS17 and CHN2; RGS17 and LRRFIP1; RGS17 and SERGEF; RGS17 and ATP8A2; RGS17 and NDRG1; RGS17 and CDC42SE1; RGS17 and LUZP2; RGS17 and HNF1B; RGS17 and TFAP2A; RGS17 and ANKRD34B; MAP7; MAP7 and C22orf36; MAP7 and NKAIN1; MAP7 and CHN2; MAP7 and LRRFIP1; MAP7 and SERGEF; MAP7 and ATP8A2; MAP7 and NDRG1; MAP7 and CDC42SE1; MAP7 and LUZP2; MAP7 and HNF1B; MAP7 and TFAP2A; MAP7 and ANKRD34B; C22orf36; C22orf36 and NKAIN1; C22orf36 and CHN2; C22orf36 and LRRFIP1; C22orf36 and SERGEF; C22orf36 and ATP8A2; C22orf36 and NDRG1; C22orf36 and CDC42SE1; C22orf36 and LUZP2; C22orf36 and HNF1B; C22orf36 and TFAP2A; C22orf36 and ANKRD34B; NKAIN1; NKAIN1 and CHN2; NKAIN1 and LRRFIP1; NKAIN1 and SERGEF; NKAIN1 and ATP8A2; NKAIN1 and NDRG1; NKAIN1 and CDC42SE1; NKAIN1 and LUZP2; NKAIN1 and HNF1B; NKAIN1 and TFAP2A; NKAIN1 and ANKRD34B; CHN2; CHN2 and LRRFIP1; CHN2 and SERGEF; CHN2 and ATP8A2; CHN2 and NDRG1; CHN2 and CDC42SE1; CHN2 and LUZP2; CHN2 and HNF1B; CHN2 and TFAP2A; CHN2 and ANKRD34B; LRRFIP1; LRRFIP1 and SERGEF; LRRFIP1 and ATP8A2; LRRFIP1 and NDRG1; LRRFIP1 and CDC42SE1; LRRFIP1 and LUZP2; LRRFIP1 and HNF1B; LRRFIP1 and TFAP2A; LRRFIP1 and ANKRD34B; SERGEF; SERGEF and ATP8A2; SERGEF and NDRG1; SERGEF and CDC42SE1; SERGEF and LUZP2; SERGEF and HNF1B; SERGEF and TFAP2A; SERGEF and ANKRD34B; ATP8A2; ATP8A2 and NDRG1; ATP8A2 and CDC42SE1; ATP8A2 and LUZP2; ATP8A2 and HNF1B; ATP8A2 and TFAP2A; ATP8A2 and ANKRD34B; NDRG1; NDRG1 and CDC42SE1; NDRG1 and LUZP2; NDRG1 and HNF1B; NDRG1 and TFAP2A; NDRG1 and ANKRD34B; CDC42SE1; CDC42SE1 and LUZP2; CDC42SE1 and HNF1B; CDC42SE1 and TFAP2A; CDC42SE1 and ANKRD34B; LUZP2; LUZP2 and HNF1B; LUZP2 and TFAP2A; LUZP2 and ANKRD34B; HNF1B; HNF1B and TFAP2A; HNF1B and ANKRD34B; TFAP2A; TFAP2A and ANKRD34B; ANKRD34B; SLC12A2, PRAC, SLC5A4, ACSL3, CD24P4, DNASE2B, SLC22A3, ODC1, SMOC2, UGDH, DSC2, WNK2, RAB3B, FAM198B, KCNC2 and SNAP91; SLC12A2; SLC12A2 and PRAC; SLC12A2 and SLC5A4; SLC12A2 and ACSL3; SLC12A2 and CD24P4; SLC12A2 and DNASE2B; SLC12A2 and SLC22A3; SLC12A2 and ODC1; SLC12A2 and SMOC2; SLC12A2 and UGDH; SLC12A2 and DSC2; SLC12A2 and WNK2; SLC12A2 and RAB3B; SLC12A2 and FAM198B; SLC12A2 and KCNC2; SLC12A2 and SNAP91; PRAC; PRAC and SLC5A4; PRAC and ACSL3; PRAC and CD24P4; PRAC and DNASE2B; PRAC and SLC22A3; PRAC and ODC1; PRAC and SMOC2; PRAC and UGDH; PRAC and DSC2; PRAC and WNK2; PRAC and RAB3B; PRAC and FAM198B; PRAC and KCNC2; PRAC and SNAP91; SLC5A4; SLC5A4 and ACSL3; SLC5A4 and CD24P4; SLC5A4 and DNASE2B; SLC5A4 and SLC22A3; SLC5A4 and ODC1; SLC5A4 and SMOC2; SLC5A4 and UGDH; SLC5A4 and DSC2; SLC5A4 and WNK2; SLC5A4 and RAB3B; SLC5A4 and FAM198B; SLC5A4 and KCNC2; SLC5A4 and SNAP91; ACSL3; ACSL3 and CD24P4; ACSL3 and DNASE2B; ACSL3 and SLC22A3; ACSL3 and ODC1; ACSL3 and SMOC2; ACSL3 and UGDH; ACSL3 and DSC2; ACSL3 and WNK2; ACSL3 and RAB3B; ACSL3 and FAM198B; ACSL3 and KCNC2; ACSL3 and SNAP91; CD24P4; CD24P4 and DNASE2B; CD24P4 and SLC22A3; CD24P4 and ODC1; CD24P4 and SMOC2; CD24P4 and UGDH; CD24P4 and DSC2; CD24P4 and WNK2; CD24P4 and RAB3B; CD24P4 and FAM198B; CD24P4 and KCNC2; CD24P4 and SNAP91; DNASE2B; DNASE2B and SLC22A3; DNASE2B and ODC1; DNASE2B and SMOC2; DNASE2B and UGDH; DNASE2B and DSC2; DNASE2B and WNK2; DNASE2B and RAB3B; DNASE2B and FAM198B; DNASE2B and KCNC2; DNASE2B and SNAP91; SLC22A3; SLC22A3 and ODC1; SLC22A3 and SMOC2; SLC22A3 and UGDH; SLC22A3 and DSC2; SLC22A3 and WNK2; SLC22A3 and RAB3B; SLC22A3 and FAM198B; SLC22A3 and KCNC2; SLC22A3 and SNAP91; ODC1; ODC1 and SMOC2; ODC1 and UGDH; ODC1 and DSC2; ODC1 and WNK2; ODC1 and RAB3B; ODC1 and FAM198B; ODC1 and KCNC2; ODC1 and SNAP91; SMOC2; SMOC2 and UGDH; SMOC2 and DSC2; SMOC2 and WNK2; SMOC2 and RAB3B; SMOC2 and FAM198B; SMOC2 and KCNC2; SMOC2 and SNAP91; UGDH; UGDH and DSC2; UGDH and WNK2; UGDH and RAB3B; UGDH and FAM198B; UGDH and KCNC2; UGDH and SNAP91; DSC2; DSC2 and WNK2; DSC2 and RAB3B; DSC2 and FAM198B; DSC2 and KCNC2; DSC2 and SNAP91; WNK2; WNK2 and RAB3B; WNK2 and FAM198B; WNK2 and KCNC2; WNK2 and SNAP91; RAB3B; RAB3B and FAM198B; RAB3B and KCNC2; RAB3B and SNAP91; FAM198B; FAM198B and KCNC2; FAM198B and SNAP91; KCNC2; KCNC2 and SNAP91; SNAP91; FAM65B, AMACR, ZNF385B, CDK19, ARHGAP18, IL5RA, SLC16A1, CNTLN, FKBP10, SLC45A2, CLIP1, HEXB, NEFH, ODZ1 and SS18L2; FAM65B; FAM65B and AMACR; FAM65B and ZNF385B; FAM65B and CDK19; FAM65B and ARHGAP18; FAM65B and IL5RA; FAM65B and SLC16A1; FAM65B and CNTLN; FAM65B and FKBP10; FAM65B and SLC45A2; FAM65B and CLIP1; FAM65B and HEXB; FAM65B and NEFH; FAM65B and ODZ1; FAM65B and SS18L2; AMACR; AMACR and ZNF385B; AMACR and CDK19; AMACR and ARHGAP18; AMACR and IL5RA; AMACR and SLC16A1; AMACR and CNTLN; AMACR and FKBP10; AMACR and SLC45A2; AMACR and CLIP1; AMACR and HEXB; AMACR and NEFH; AMACR and ODZ1; AMACR and SS18L2; ZNF385B; ZNF385B and CDK19; ZNF385B and ARHGAP18; ZNF385B and IL5RA; ZNF385B and SLC16A1; ZNF385B and CNTLN; ZNF385B and FKBP10; ZNF385B and SLC45A2; ZNF385B and CLIP1; ZNF385B and HEXB; ZNF385B and NEFH; ZNF385B and ODZ1; ZNF385B and SS18L2; CDK19; CDK19 and ARHGAP18; CDK19 and IL5RA; CDK19 and SLC16A1; CDK19 and CNTLN; CDK19 and FKBP10; CDK19 and SLC45A2; CDK19 and CLIP1; CDK19 and HEXB; CDK19 and NEFH; CDK19 and ODZ1; CDK19 and SS18L2; ARHGAP18; ARHGAP18 and IL5RA; ARHGAP18 and SLC16A1; ARHGAP18 and CNTLN; ARHGAP18 and FKBP10; ARHGAP18 and SLC45A2; ARHGAP18 and CLIP1; ARHGAP18 and HEXB; ARHGAP18 and NEFH; ARHGAP18 and ODZ1; ARHGAP18 and SS18L2; IL5RA; IL5RA and SLC16A1; IL5RA and CNTLN; IL5RA and FKBP10; IL5RA and SLC45A2; IL5RA and CLIP1; IL5RA and HEXB; IL5RA and NEFH; IL5RA and ODZ1; IL5RA and SS18L2; SLC16A1; SLC16A1 and CNTLN; SLC16A1 and FKBP10; SLC16A1 and SLC45A2; SLC16A1 and CLIP1; SLC16A1 and HEXB; SLC16A1 and NEFH; SLC16A1 and ODZ1; SLC16A1 and SS18L2; CNTLN; CNTLN and FKBP10; CNTLN and SLC45A2; CNTLN and CLIP1; CNTLN and HEXB; CNTLN and NEFH; CNTLN and ODZ1; CNTLN and SS18L2; FKBP10; FKBP10 and SLC45A2; FKBP10 and CLIP1; FKBP10 and HEXB; FKBP10 and NEFH; FKBP10 and ODZ1; FKBP10 and SS18L2; SLC45A2; SLC45A2 and CLIP1; SLC45A2 and HEXB; SLC45A2 and NEFH; SLC45A2 and ODZ1; SLC45A2 and SS18L2; CLIP1; CLIP1 and HEXB; CLIP1 and NEFH; CLIP1 and ODZ1; CLIP1 and SS18L2; HEXB; HEXB and NEFH; HEXB and ODZ1; HEXB and SS18L2; NEFH; NEFH and ODZ1; NEFH and SS18L2; ODZ1; ODZ1 and SS18L2; SS18L2; HPGD, FAM3B, MIPEP, NCAPD3, INPP4B, ANPEP, TFF3, IL31RA, EHHADH, RP11-45B20.2, CCDC141, RLN1, ABHD2 and SCIN; HPGD; HPGD and FAM3B; HPGD and MIPEP; HPGD and NCAPD3; HPGD and INPP4B; HPGD and ANPEP; HPGD and TFF3; HPGD and IL31RA; HPGD and EHHADH; HPGD and RP11-45B20.2; HPGD and CCDC141; HPGD and RLN1; HPGD and ABHD2; HPGD and SCIN; FAM3B; FAM3B and MIPEP; FAM3B and NCAPD3; FAM3B and INPP4B; FAM3B and ANPEP; FAM3B and TFF3; FAM3B and IL31RA; FAM3B and EHHADH; FAM3B and RP11-45B20.2; FAM3B and CCDC141; FAM3B and RLN1; FAM3B and ABHD2; FAM3B and SCIN; MIPEP; MIPEP and NCAPD3; MIPEP and INPP4B; MIPEP and ANPEP; MIPEP and TFF3; MIPEP and IL31RA; MIPEP and EHHADH; MIPEP and RP11-45B20.2; MIPEP and CCDC141; MIPEP and RLN1; MIPEP and ABHD2; MIPEP and SCIN; NCAPD3; NCAPD3 and INPP4B; NCAPD3 and ANPEP; NCAPD3 and TFF3; NCAPD3 and IL31RA; NCAPD3 and EHHADH; NCAPD3 and RP11-45B20.2; NCAPD3 and CCDC141; NCAPD3 and RLN1; NCAPD3 and ABHD2; NCAPD3 and SCIN; INPP4B; INPP4B and ANPEP; INPP4B and TFF3; INPP4B and IL31RA; INPP4B and EHHADH; INPP4B and RP11-45B20.2; INPP4B and CCDC141; INPP4B and RLN1; INPP4B and ABHD2; INPP4B and SCIN; ANPEP; ANPEP and TFF3; ANPEP and IL31RA; ANPEP and EHHADH; ANPEP and RP11-45B20.2; ANPEP and CCDC141; ANPEP and RLN1; ANPEP and ABHD2; ANPEP and SCIN; TFF3; TFF3 and IL31RA; TFF3 and EHHADH; TFF3 and RP11-45B20.2; TFF3 and CCDC141; TFF3 and RLN1; TFF3 and ABHD2; TFF3 and SCIN; IL31RA; IL31RA and EHHADH; IL31RA and RP11-45B20.2; IL31RA and CCDC141; IL31RA and RLN1; IL31RA and ABHD2; IL31RA and SCIN; EHHADH; EHHADH and RP11-45B20.2; EHHADH and CCDC141; EHHADH and RLN1; EHHADH and ABHD2; EHHADH and SCIN; RP11-45B20.2; RP11-45B20.2 and CCDC141; RP11-45B20.2 and RLN1; RP11-45B20.2 and ABHD2; RP11-45B20.2 and SCIN; CCDC141; CCDC141 and RLN1; CCDC141 and ABHD2; CCDC141 and SCIN; RLN1; RLN1 and ABHD2; RLN1 and SCIN; ABHD2; ABHD2 and SCIN; SCIN; TFF3, ALOX15B, and MON1B; TFF3 and ALOX15B; TFF3 and MON1B; ALOX15B; ALOX15B, and MON1B; MON1B; MME, BANK1, LEPREL1, VGLL3, NPR3, OR4K7P, OR4K6P, POTEB2, RP11, TTN, FAP5 and GPR116; MME; MME and BANK1; MME and LEPREL1; MME and VGLL3; MME and NPR3; MME and OR4K7P; MME and OR4K6P; MME and POTEB2; MME and RP11; MME and TTN; MME and FAP5; MME and GPR116; BANK1; BANK1 and LEPREL1; BANK1 and VGLL3; BANK1 and NPR3; BANK1 and OR4K7P; BANK1 and OR4K6P; BANK1 and POTEB2; BANK1 and RP11; BANK1 and TTN; BANK1 and FAP5; BANK1 and GPR116; LEPREL1; LEPREL1 and VGLL3; LEPREL1 and NPR3; LEPREL1 and OR4K7P; LEPREL1 and OR4K6P; LEPREL1 and POTEB2; LEPREL1 and RP11; LEPREL1 and TTN; LEPREL1 and FAP5; LEPREL1 and GPR116; VGLL3; VGLL3 and NPR3; VGLL3 and OR4K7P; VGLL3 and OR4K6P; VGLL3 and POTEB2; VGLL3 and RP11; VGLL3 and TTN; VGLL3 and FAP5; VGLL3 and GPR116; NPR3; NPR3 and OR4K7P; NPR3 and OR4K6P; NPR3 and POTEB2; NPR3 and RP11; NPR3 and TTN; NPR3 and FAP5; NPR3 and GPR116; OR4K7P; OR4K7P and OR4K6P; OR4K7P and POTEB2; OR4K7P and RP11; OR4K7P and TTN; OR4K7P and FAP5; OR4K7P and GPR116; OR4K6P; OR4K6P and POTEB2; OR4K6P and RP11; OR4K6P and TTN; OR4K6P and FAP5; OR4K6P and GPR116; POTEB2; POTEB2 and RP11; POTEB2 and TTN; POTEB2 and FAP5; POTEB2 and GPR116; RP11; RP11 and TTN; RP11 and FAP5; RP11 and GPR116; TTN; TTN and FAP5; TTN and GPR116; FAP5; FAP5 and GPR116; GPR116; MME, BANK1, LEPREL1, VGLL3, NPR3, OR4K6P, OR4K7P, POTEB2, RP11.403, TTN and FABP5P7; RP11.403; RP11.403 and TTN; RP11.403 and FABP5P7; TTN; TTN and FABP5P7; and FABP5P7. The plurality of targets may comprise 5, 6, 7, 8, 9, 10, 11, 12, 13, 14, 15, 20, 30, 40, 50 or more targets.

The plurality of targets may comprise CDC20; KIF2C; PHGDH; NUF2; CENPF; EXO1; UBE2T; RRM2; MLPH; GPR160; CCNB1; CXXC5; PTTG1; FGFR4; FOXC1; ESR1; ANLN; BLVRA; EGFR; ACTR3B; NAT1; MYC; SFRP1; MELK; BAG1; CEP55; MKI67; TMEM45B; PGR; MDM2; KRT5; FOXA1; ORC6; CDH3; ERBB2; GRB7; CDC6; MAPT; BIRC5; KRT14; KRT17; TYMS; NDC80; SLC39A6; BCL2; CCNE1; MIA; MYBL2; UBE2C; MMP11; TDRD1; CACNA1D; NCALD; HLA-DMB; KCNH8; PDE3B; PLA2G7; CSGALNACT1; PART1; HES1; F3; GPR110; SH3RF; PDE8B; SEPT9; CRISP3; AMD1; KCNG3; PLA1A; MYO6; FRK; SH3YL1; ACER3; C8orf4; GHR; ITPR1; KHDRBS3; NPY; GUCY1A3; ARHGDIB; LAMC2; VWA2; ZNF432; MORN1; CYorf15B; AMPD3; QDPR; HDAC1; KIF16B; GJB1; ITPR3; ZNF615; ANKRD6; APOD; STEAP4; RGS17; MAP7; C22orf36; NKAIN1; CHN2; LRRFIP1; SERGEF; ATP8A2; NDRG1; CDC42SE1; LUZP2; HNF1B; TFAP2A; ANKRD34B; SLC12A2; PRAC; SLC5A4; ACSL3; CD24P4; DNASE2B; SLC22A3; ODC1; SMOC2; UGDH; DSC2; WNK2; RAB3B; FAM198B; KCNC2; SNAP91; FAM65B; AMACR; ZNF385B; CDK19; ARHGAP18; IL5RA; SLC16A1; CNTLN; FKBP10; SLC45A2; CLIP1; HEXB; NEFH; ODZ1; SS18L2; HPGD; FAM3B; MIPEP; NCAPD3; INPP4B; ANPEP; TFF3; IL31RA; EHHADH; RP11-45B20.2; CCDC141; RLN1; ABHD2; SCIN; ALOX15B; MON1B; MME; BANK1; LEPREL1; VGLL3; NPR3; OR4K7P; OR4K6P; POTEB2; RP11; TTN; FAP5; GPR116; RP11.403; and FABP5P7.

### Probes/Primers

The present invention provides for a probe set for diagnosing, monitoring and/or predicting a status or outcome of a prostate cancer in a subject comprising a plurality of probes, wherein (i) the probes in the set are capable of detecting an expression level of at least one target selected from; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from prostate cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In some embodiments, the probe set comprise a plurality of target sequences that hybridize to at least about 5 coding targets and/or non-coding targets selected from Table 8, Table 9 or SEQ ID NOs: 1-1029. Alternatively, the probe set comprise a plurality of target sequences that hybridize to at least about 10 coding targets and/or non-coding targets selected from Table 8, Table 9 or SEQ ID NOs: 1-1029. In some embodiments, the probe set comprise a plurality of target sequences that hybridize to at least about 15 coding targets and/or non-coding targets selected from Table 8, Table 9 or SEQ ID NOs: 1-1029. In some embodiments, the probe set comprise a plurality of target sequences that hybridize to at least about 20 coding targets and/or non-coding targets selected from Table 8, Table 9 or SEQ ID NOs: 1-1029. In some embodiments, the probe set comprise a plurality of target sequences that hybridize to at least about 30 coding targets and/or non-coding targets selected from Table 8, Table 9 or SEQ ID NOs: 1-1029. In some embodiments, the probe set comprise a plurality of target sequences that hybridize to at least about 40 coding targets and/or non-coding targets selected from Table 8, Table 9 or SEQ ID NOs: 1-1029. In some embodiments, the probe set comprise a plurality of target sequences that hybridize to at least about 50 coding targets and/or non-coding targets selected from Table 8, Table 9 or SEQ ID NOs: 1-1029.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target selected from Table 8, Table 9 or SEQ ID NOs: 1-1029 (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the invention comprise at least 20 consecutive bases of the nucleic acid sequence of a target selected from Table 8, Table 9 or SEQ ID NOs: 1-1029 or a complement thereto. The polynucleotides may comprise at least 21, 22, 23, 24, 25, 27, 30, 32, 35 or more consecutive bases of the nucleic acids sequence of a target selected from Table 8, Table 9 or SEQ ID NOs: 1-1029, as applicable.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly) vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

Diagnostic samples for use with the systems and in the methods of the present invention comprise nucleic acids suitable for providing RNAs expression information. In principle, the biological sample from which the expressed RNA is obtained and analyzed for target sequence expression can be any material suspected of comprising prostate cancer tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising cancer tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly MA, High Pure FFPE RNA Micro Kit, Roche Applied Science, Indianapolis, IN). RNA can be extracted from frozen tissue sections using TRIzol (Invitrogen, Carlsbad, CA) and purified using RNeasy Protect kit (Qiagen, Valencia, CA). RNA can be further purified using DNAse I treatment (Ambion, Austin, TX) to eliminate any contaminating DNA. RNA concentrations can be made using a Nanodrop ND-1000 spectrophotometer (Nanodrop Technologies, Rockland, DE). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, CA).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100 or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 8, Table 9 or SEQ ID NOs: 1-1029, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 8, Table 9 or SEQ ID NOs: 1-1029, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, CA), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp Plus RNA Amplification Kits (MDS Analytical Technologies (formerly Arcturus) (Mountain View, CA), Genisphere, Inc. (Hatfield, PA), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, CA), SmartCycler® 9600 or GeneXpert® Systems (Cepheid, Sunnyvale, CA), ABI 7900 HT Fast Real Time PCR system (Applied Biosystems, Foster City, CA), LightCycler® 480 System (Roche Molecular Systems, Pleasanton, CA), xMAP 100 System (Luminex, Austin, TX) Solexa Genome Analysis System (Illumina, Hayward, CA), OpenArray Real Time qPCR (BioTrove, Woburn, MA) and BeadXpress System (Illumina, Hayward, CA).

Detection and/or Quantification of Target Sequences

Any method of detecting and/or quantitating the expression of the encoded target sequences can in principle be used in the invention. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLID sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for QRT-PCR Analysis

Reverse transcription can be performed by any method known in the art. For example, reverse transcription may be performed using the Omniscript kit (Qiagen, Valencia, CA), Superscript III kit (Invitrogen, Carlsbad, CA), for RT-PCR. Target-specific priming can be performed in order to increase the sensitivity of detection of target sequences and generate target-specific cDNA.

### TagMan® Gene Expression Analysis

TaqMan® RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present invention contemplates that a probe set or probes derived therefrom may be provided in an array format. In the context of the present invention, an “array” is a spatially or logically organized collection of polynucleotide probes. An array comprising probes specific for a coding target, non-coding target, or a combination thereof may be used. Alternatively, an array comprising probes specific for two or more of transcripts of a target selected from Table 8, Table 9 or SEQ ID NOs: 1-1029 or a product derived thereof can be used. Desirably, an array may be specific for 5, 10, 15, 20, 25, 30, 40, 50 or more of transcripts of a target selected from Table 8, Table 9 or SEQ ID NOs: 1-1029. Expression of these sequences may be detected alone or in combination with other transcripts. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, CA.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly) vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the Cancer Prognostic array is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, IRMA, Probe Logarithmic Intensity Error (PLIER) algorithm or variant iterPLIER. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence e Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Cancer

The systems, compositions and methods disclosed herein may be used to diagnosis, monitor and/or predict the status or outcome of a cancer. Generally, a cancer is characterized by the uncontrolled growth of abnormal cells anywhere in a body. The abnormal cells may be termed cancer cells, malignant cells, or tumor cells. Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

### Cancer Staging

Diagnosing, predicting, or monitoring a status or outcome of a cancer may comprise determining the stage of the cancer. Generally, the stage of a cancer is a description (usually numbers I to IV with IV having more progression) of the extent the cancer has spread. The stage often takes into account the size of a tumor, how deeply it has penetrated, whether it has invaded adjacent organs, how many lymph nodes it has metastasized to (if any), and whether it has spread to distant organs. Staging of cancer can be used as a predictor of survival, and cancer treatment may be determined by staging. Determining the stage of the cancer may occur before, during, or after treatment. The stage of the cancer may also be determined at the time of diagnosis.

Cancer staging can be divided into a clinical stage and a pathologic stage. Cancer staging may comprise the TNM classification. Generally, the TNM Classification of Malignant Tumours (TNM) is a cancer staging system that describes the extent of cancer in a patient's body. T may describe the size of the tumor and whether it has invaded nearby tissue, N may describe regional lymph nodes that are involved, and M may describe distant metastasis (spread of cancer from one body part to another). In the TNM (Tumor, Node, Metastasis) system, clinical stage and pathologic stage are denoted by a small “c” or “p” before the stage (e.g., cT3N1M0 or pT2N0).

Often, clinical stage and pathologic stage may differ. Clinical stage may be based on all of the available information obtained before a surgery to remove the tumor. Thus, it may include information about the tumor obtained by physical examination, radiologic examination, and endoscopy. Pathologic stage can add additional information gained by examination of the tumor microscopically by a pathologist. Pathologic staging can allow direct examination of the tumor and its spread, contrasted with clinical staging which may be limited by the fact that the information is obtained by making indirect observations at a tumor which is still in the body. The TNM staging system can be used for most forms of cancer.

Alternatively, staging may comprise Ann Arbor staging. Generally, Ann Arbor staging is the staging system for lymphomas, both in Hodgkin's lymphoma (previously called Hodgkin's disease) and Non-Hodgkin lymphoma (abbreviated NHL). The stage may depend on both the place where the malignant tissue is located (as located with biopsy, CT scanning and increasingly positron emission tomography) and on systemic symptoms due to the lymphoma (“B symptoms”: night sweats, weight loss of >10% or fevers). The principal stage may be determined by location of the tumor. Stage I may indicate that the cancer is located in a single region, usually one lymph node and the surrounding area. Stage I often may not have outward symptoms. Stage II can indicate that the cancer is located in two separate regions, an affected lymph node or organ and a second affected area, and that both affected areas are confined to one side of the diaphragm—that is, both are above the diaphragm, or both are below the diaphragm. Stage III often indicates that the cancer has spread to both sides of the diaphragm, including one organ or area near the lymph nodes or the spleen. Stage IV may indicate diffuse or disseminated involvement of one or more extralymphatic organs, including any involvement of the liver, bone marrow, or nodular involvement of the lungs.

Modifiers may also be appended to some stages. For example, the letters A, B, E, X, or S can be appended to some stages. Generally, A or B may indicate the absence of constitutional (B-type) symptoms is denoted by adding an “A” to the stage; the presence is denoted by adding a “B” to the stage. E can be used if the disease is “extranodal” (not in the lymph nodes) or has spread from lymph nodes to adjacent tissue. X is often used if the largest deposit is >10 cm large (“bulky disease”), or whether the mediastinum is wider than ⅓ of the chest on a chest X-ray. S may be used if the disease has spread to the spleen.

The nature of the staging may be expressed with CS or PS. CS may denote that the clinical stage as obtained by doctor's examinations and tests. PS may denote that the pathological stage as obtained by exploratory laparotomy (surgery performed through an abdominal incision) with splenectomy (surgical removal of the spleen).

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For patients with high test scores consistent with systemic disease outcome after prostatectomy, additional treatment modalities such as adjuvant chemotherapy (e.g., docetaxel, mitoxantrone and prednisone), systemic radiation therapy (e.g., samarium or strontium) and/or anti-androgen therapy (e.g., surgical castration, finasteride, dutasteride) can be designated. Such patients would likely be treated immediately with anti-androgen therapy alone or in combination with radiation therapy in order to eliminate presumed micro-metastatic disease, which cannot be detected clinically but can be revealed by the target sequence expression signature.

Such patients can also be more closely monitored for signs of disease progression. For patients with intermediate test scores consistent with biochemical recurrence only (BCR-only or elevated PSA that does not rapidly become manifested as systemic disease only localized adjuvant therapy (e.g., radiation therapy of the prostate bed) or short course of anti-androgen therapy would likely be administered. For patients with low scores or scores consistent with no evidence of disease (NED) adjuvant therapy would not likely be recommended by their physicians in order to avoid treatment-related side effects such as metabolic syndrome (e.g., hypertension, diabetes and/or weight gain), osteoporosis, proctitis, incontinence or impotence. Patients with samples consistent with NED could be designated for watchful waiting, or for no treatment. Patients with test scores that do not correlate with systemic disease but who have successive PSA increases could be designated for watchful waiting, increased monitoring, or lower dose or shorter duration anti-androgen therapy.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty, forty, fifty or more of the target sequences corresponding to a target selected from Table 8, Table 9 or SEQ ID NOs: 1-1029, the subsets described herein, or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms can assist in the visualization of such data.

### Subtyping

The inventors of the present invention discovered that luminal and basal subtyping of prostate cancer is prognostic and predicts response to androgen deprivation therapy. Prostate cancer subtypes useful in the methods of the present invention include, for example, luminal A, luminal B, and basal subtypes. Molecular subtyping is a method of classifying prostate cancers into one of multiple genetically-distinct categories, or subtypes. Each subtype responds differently to different kinds of treatments, and some subtypes indicate a higher risk of recurrence. Subtypes of the present invention may be used to predict outcomes such as distant metastasis-free survival (DMFS), biochemical recurrence-free survival (bRFS), prostate cancer specific survival (PCSS), and overall survival (OS). As described herein, each subtype has a unique molecular and clinical fingerprint. The subtyping methods described herein may be used to predict prostate cancer response to androgen deprivation therapy.

Differential expression analysis one or more of the targets listed in Table 8, Table 9 or SEQ ID NOs: 1-1029 allow for the identification of the molecular subtype of a prostate cancer.

In some instances, the molecular subtyping methods of the present invention are used in combination with other biomarkers, like tumor grade and hormone levels, for analyzing the prostate cancer.

### Clinical Associations and Patient Outcomes

Molecular subtypes of the present invention have distinct clinical associations. Clinical associations that correlate to molecular subtypes include, for example, preoperative serum PSA, Gleason score (GS), extraprostatic extension (EPE), surgical margin status (SM), lymph node involvement (LNI), and seminal vesicle invasion (SVI).

In some embodiments, molecular subtypes of the present invention are used to predict patient outcomes such as biochemical recurrence (BCR), metastasis (MET) and prostate cancer death (PCSM) after radical prostatectomy. In other embodiments, molecular subtypes of the present invention are used to predict patient outcomes such as distant metastasis-free survival (DMFS), biochemical recurrence-free survival (bRFS), prostate cancer specific survival (PCSS), and overall survival (OS).

### Treatment Response Prediction

In some embodiments, the molecular subtypes of the present invention are useful for predicting response to Androgen Deprivation Therapy (ADT) following radical prostatectomy. Androgen deprivation therapy (ADT), also called androgen suppression therapy, is an antihormone therapy whose main use is in treating prostate cancer. Prostate cancer cells usually require androgen hormones, such as testosterone, to grow. ADT reduces the levels of androgen hormones, with drugs or surgery, to prevent the prostate cancer cells from growing. The pharmaceutical approaches include antiandrogens and chemical castration.

In other embodiments, the molecular subtypes of the present invention are useful for predicting response to Radiation Therapy (RT) following radical prostatectomy.

### EXAMPLES

### Example 1: A Genetic Classifier (PAM50) to Identify Subtypes in Prostate Cancer and Predict Response to Therapy

A genetic signature (PAM50) to identify subtypes in prostate cancer tissue and predict response to therapy was developed as follows. Affymetrix Human Exon 1.0 ST microarray (Affymetrix, Santa Clara, CA) data from formalin-fixed paraffin-embedded radical prostatectomy samples were obtained from six published retrospective patient cohorts (n=1,567) and one prospective cohort (n=2,215) for a total of 3,782 samples. Retrospective cohorts were from the Mayo Clinic (MC I and II), Cleveland Clinic (CC), Johns Hopkins University (JHU), Thomas Jefferson University (TJU), and Durham VA (DVA). Data collection was approved and supervised by local institutional review boards (IRB). 2,215 de-identified, anonymized, and prospectively-collected patients from clinical use of the Decipher test were obtained from Decipher GRID™ (ClinicalTrials.gov ID: NCT02609269). Clinical outcomes were not available for Decipher GRID. Informed consent and IRB approval for Decipher GRID were obtained. Microarray processing was performed in a CLIA-certified clinical operations laboratory (GenomeDx Biosciences, Inc, San Diego, CA). Microarrays were normalized using Single Channel Array Normalization. (Piccolo et al. Genomics. 2012; 100 (6): 337-344.)

### PAM50 Clustering

PAM50 clustering was performed based on the original algorithm from Parker et al. J Clin Oncol. 2009; 27 (8): 1160-1167. Source code was downloaded from [https://genome.unc.edu/pubsup/breastGEO/] and used without modification. Gene expression data were median-centered in each cohort individually as required by the PAM50 algorithm. The normal-like subtype was excluded as the prostate cancer samples were macro-dissected limiting the amount of normal tissue present in our data. The HER2 subtype was also excluded given the lack of HER2 amplification in prostate cancer (Ullen et al. Acta Oncol. 2005;44 (5): 490-495). Assignment of subtype in the prostate cancer samples was thus assigned by the greatest correlation with luminal A, luminal B, or basal.

### Clinical Endpoints

All primary and secondary endpoints were preplanned. The primary clinical endpoint was distant metastasis-free survival (DMFS), with secondary clinical endpoints of biochemical recurrence-free survival (BRFS), prostate cancer specific survival (PCSS), and overall survival (OS). All endpoints were defined from time of surgery until time of the event, death, or last follow-up.

### Gene Set Enrichment Analysis

Functional and biological analyses of the PAM50 subtypes in prostate cancer were investigated using Gene Set Enrichment Analysis (GSEA). First, a T-test was performed on every gene comparing expression in the specified subtype vs. not in that subtype. The T-statistic was used to generate a pre-ranked list which was input into GSEA.

### Matched Cohort Design and Predicting Response to ADT

To investigate if subtype could predict ADT response, a matched cohort with 2:1 matching for ADT untreated and treated patients was created from the MCI and MCII cohorts in order to select patients from a single institution with a mix of post-operatively treated and untreated patients. This resulted in a cohort of 315 patients, 210 of which did not receive any ADT, and 105 which received ADT treatment. The decision to perform 2:1 matching was to maximize sample size using patients only from the MC cohorts. We chose to only include patients from the MC cohorts for this analysis because patients in these cohorts received a mix of adjuvant and salvage ADT and RT, allowing us to account for the effects of both in our models. JHMI patients did not receive any post-operative treatment. CCF patients did not receive adjuvant treatment, and information about salvage ADT treatment was unavailable in the dataset. All TJU and DVA patients were treated with radiation. We defined androgen deprivation therapy (ADT) as treatment (with LHRH agonist alone or in combination with androgen receptor antagonists) after radical prostatectomy but before the primary endpoint of metastasis. Matching was performed based on Gleason, prostate specific antigen (PSA, ng/ml), positive surgical margins (SM), extracapsular extension (ECE), seminal vesicle invasion (SVI), lymph node invasion (LNI), as well as post-operative radiation therapy (RT). Data on the duration and dose of ADT were not available. Table 7 provides details of which patients in this matched cohort received adjuvant, salvage, or both ADT and/or RT. Nearly all lymph node positive patients from the MC cohorts received ADT, as well as some who received ADT for other reasons at the treating physicians' discretion.

TABLE 7
Number of patients receiving ADT and RT in
the matched cohort (n = 315)

| | Adjuvant | Salvage | Both | |
|---|---|---|---|---|
| | ADT | ADT | Adjuvant and | No |
| | Only | Only | Salvage ADT | ADT |
| Adjuvant RT | 4 | 6 | 2 | 23 |
| Only | | | | |
| Salvage RT Only | 3 | 14 | 2 | 41 |
| Both Adjuvant | 1 | 0 | 0 | 0 |
| and Salvage RT | | | | |
| No RT | 18 | 53 | 2 | 146 |

Abbreviations:
ADT: Androgen deprivation therapy;
RT: Radiation therapy

### Statistical Analysis

In the demographics tables, ANOVA and Chi-squared test were used to evaluate differences between continuous and categorical variables, respectively, between patient groups. Kaplan-Meier curves were generated by pooling clinical data from all available microarray cohorts. Gleason score was stratified into low (<7), intermediate (7), and high risk (8-10). PSA was stratified into low (<10 ng/mL), intermediate (10-20 ng/mL), and high risk (>20 ng/mL) in a similar manner. SM, ECE, SVI, and LNI were considered binary variables and defined by the respective institutions. Cox regression was used for both univariable and multivariable analysis (UVA/MVA). Stratification by cohort was used when performing UVA/MVA analyses to account for baseline differences between cohorts. The interaction term for treatment and subtype in a Cox model was used to evaluate prediction of treatment response, and a significant interaction Wald test p-value indicated that a subtype could predict response to ADT. Statistical significance was set as a two-tailed p-value<0.05. All statistical analyses were performed in R 3.1.2.

### Microarray Data Accession

Microarray data is available on Gene Expression Omnibus with accession numbers GSE46691, GSE62116, GSE72291, GSE62667, GSE79956, GSE79957, and GSE79915. Subtyping target sequences (SEQ ID NOs 1-1029) are shown in Table 8.

### Results

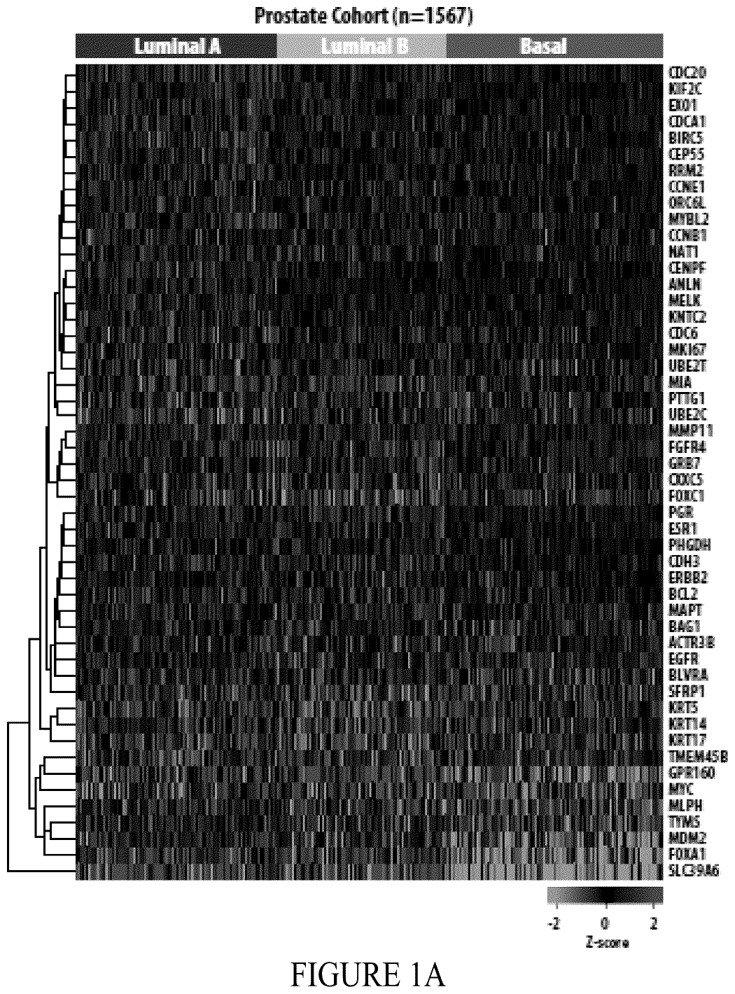

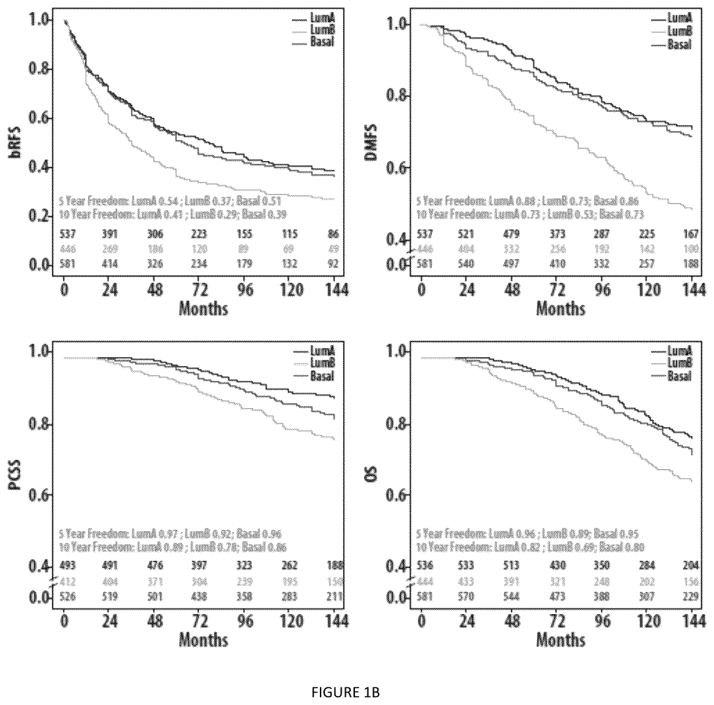

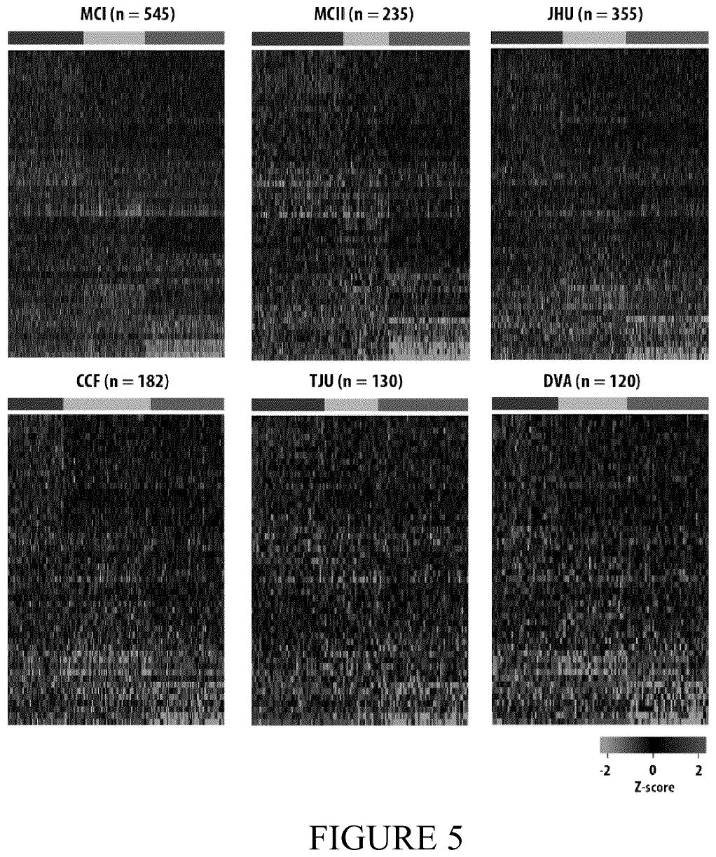

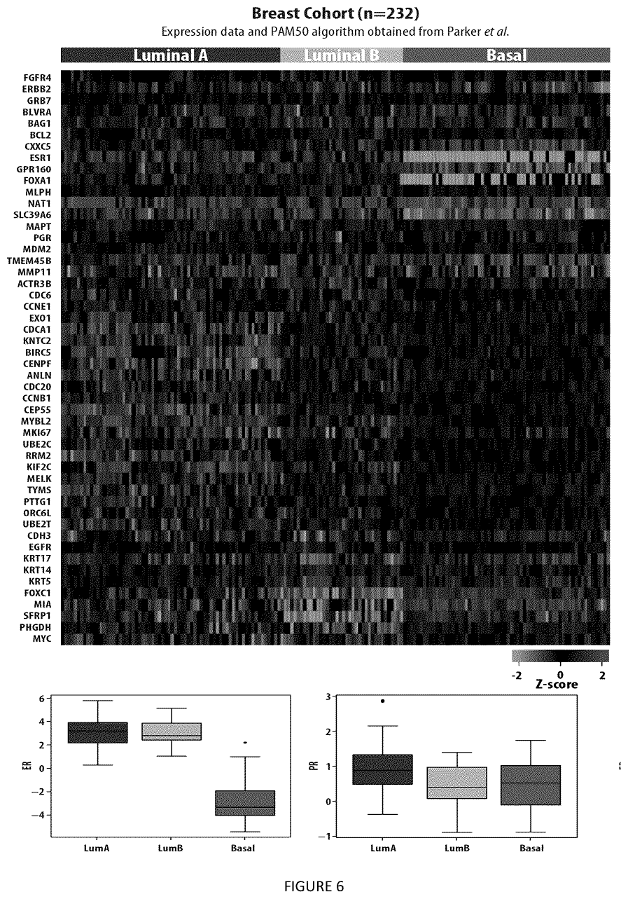

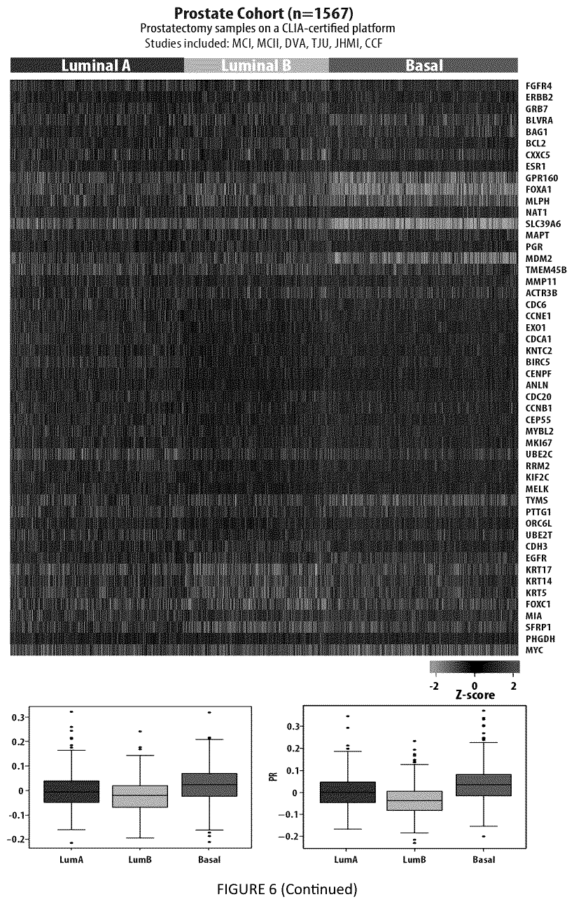

To subtype prostate cancers into luminal-like vs. basal-like subtypes, we applied the PAM50 classifier on 1,567 prostate cancer samples with a median clinical follow-up time of 10 years. 34.3% of samples are classified as luminal A, 28.5% as luminal B, and 37.1% as basal, with visually similar patterns of expression across all six independent cohorts (FIG. 1A, FIG. 5, Table 2). PAM50 expression patterns are also similar between breast and prostate cancer samples (FIG. 6). Notably, ER and PR, which are highest in luminal and luminal A breast cancer, respectively⁸, do not demonstrate the same patterns in prostate cancer (FIG. 6).

We next examined associations of luminal A, luminal B, and basal subtypes with clinical outcomes. Luminal B patients consistently have significantly worse outcomes for all endpoints compared to luminal A and basal subtypes (FIG. 1B). The 10-year actuarial rates for bRFS are 29% for luminal B compared to 41% and 39% for luminal A and basal, respectively; for DMFS, 53% for luminal B compared to 73% for both other subtypes; for PCSS, 78% for luminal B compared to 89% and 86% for luminal A and basal; and for OS, 69% for luminal B versus 82% and 80% for luminal A and basal.

On univariable Cox analysis (Table 1, Table 3), basal and luminal A have improved bRFS, DMFS, PCSS, and OS compared to luminal B (bRFS: basal (vs. luminal B) p<0.0001, HR=0.69 [0.59-0.81], luminal A p<0.0001, HR=0.66 [0.57-0.78]; DMFS: basal p<0.0001, HR=0.5 [0.4-0.61], luminal A p<0.0001, HR=0.42 [0.34-0.53]; PCSS: basal p=0.0003, HR=0.59 [0.44-0.79], luminal A p<0.0001, HR=0.38 [0.27-0.53]; OS basal p=0.0005, HR=0.69 [0.56-0.85], luminal A p<0.0001, HR=0.56 [0.45-0.7]). However, luminal A does not exhibit significantly different bRFS (p=0.61, HR=1.04 [0.89-1.22]) and DMFS (p=0.18, HR=1.17 [0.93-1.49]) versus basal. Luminal A does demonstrate worse PCSS (p=0.01, HR=1.54 [1.09-2.16]) and OS (p=0.05, HR=1.25 [1-1.55]) compared to basal, though this is difficult to interpret in the setting of non-significant differences in metastasis and biochemical recurrence. Consistent with our data demonstrating that luminal B patients have the worst clinical outcomes, luminal B patients also have the highest pre-operative PSA levels, Gleason score, and rates of ECE and LNI, followed by basal and then luminal A (Table 2). On multivariable analysis (Table 1, Table 3), adjusting for clinicopathologic variables (age, PSA, Gleason score, SM, ECE, SVI, and LNI), basal and luminal A have significantly better independent prognosis than luminal B for bRFS (basal vs. luminal B: p=0.01, HR=0.81 [0.69-0.96]; luminal A vs. luminal B: p=0.005, HR=0.79 [0.66-0.93]) and DMFS (basal vs. luminal B: p=0.0002, HR=0.66 [0.53-0.82]; luminal A vs. luminal B: p<0.0001, HR=0.55 [0.43-0.69]). Luminal A also has significantly improved outcomes compared to luminal B for PCSS (p<0.0001, HR=0.50 [0.35-0.71]) and OS (p=0.002, HR=0.69 [0.55-0.87]). To provide comparison to a composite clinical classifier, we similarly show that basal and luminal A have significantly better prognosis than luminal B for all endpoints on multivariable analysis adjusting for age, LNI, and the assessment by risk using the D'Amico classifier (D'Amico et al. JAMA. 1998;280 (11): 969-974) (See Table 4).

TABLE 1
Univariable and Multivariable Analysis

| | | | | MVA | |
|---|---|---|---|---|---|
| | | UVA | | P- | |
| | | P-value | HR [95% CI] | value | HR [95% CI] |
| DMFS | Age (yrs) | 0.88 | 1 [0.99-1.02] | 0.15 | 0.99 [0.98-1] |
| | PSA 10-20 vs. <10 | 0.64 | 1.05 [0.85-1.31] | 0.29 | 0.89 [0.71-1.11] |
| | PSA >20 vs. <10 | 3.8E−03 | 1.42 [1.12-1.79] | 0.16 | 0.83 [0.64-1.08] |
| | Gleason 7 vs. <7 | 8.4E−05 | 4.57 [2.14-9.73] | 1.3E− | 3.49 [1.63-7.47] |
| | | | | 03 | |
| | Gleason 8-10 vs. <7 | 4.0E−12 | 14.32 [6.75-30.37] | 2.4E− | 8.8 [4.1-18.88] |
| | | | | 08 | |
| | SMS | 0.08 | 1.18 [0.98-1.42] | 0.74 | 1.03 [0.85-1.25] |
| | SVI | 0.0E+00 | 2.57 [2.14-3.08] | 3.5E− | 1.72 [1.39-2.11] |
| | | | | 07 | |
| | ECE | 3.7E−12 | 2.04 [1.67-2.5] | 0.07 | 1.23 [0.99-1.54] |
| | LNI | 0.0E+00 | 2.56 [2.06-3.19] | 0.01 | 1.39 [1.09-1.78] |
| | Basal vs. LumB | 9.0E−11 | 0.5 [0.4-0.61] | 2.0E− | 0.66 [0.53-0.82] |
| | | | | 04 | |
| | LumA vs. LumB | 9.1E−14 | 0.42 [0.34-0.53] | 5.4E− | 0.55 [0.43-0.69] |
| | | | | 07 | |
| PCSS | Age (yrs) | 0.86 | 1 [0.98-1.02] | 0.24 | 0.99 [0.97-1.01] |
| | PSA 10-20 vs. <10 | 0.79 | 1.04 [0.76-1.42] | 0.16 | 0.8 [0.58-1.09] |
| | PSA >20 vs. <10 | 0.07 | 1.35 [0.97-1.86] | 0.01 | 0.62 [0.43-0.89] |
| | Gleason 7 vs. <7 | 0.02 | 3.35 [1.22-9.19] | 0.06 | 2.7 [0.98-7.46] |
| | Gleason 8-10 vs. <7 | 2.4E−07 | 13.76 [5.08-37.23] | 3.1E− | 8.6 [3.12-23.68] |
| | | | | 05 | |
| | SMS | 9.3E−04 | 1.56 [1.2-2.02] | 0.11 | 1.25 [0.95-1.64] |
| | SVI | 0.0E+00 | 3.15 [2.43-4.08] | 2.0E− | 2.06 [1.53-2.78] |
| | | | | 06 | |
| | ECE | 5.1E−08 | 2.22 [1.67-2.96] | 0.29 | 1.19 [0.87-1.63] |
| | LNI | 2.2E−15 | 3.19 [2.4-4.25] | 4.7E− | 1.6 [1.15-2.21] |
| | | | | 03 | |
| | Basal vs. LumB | 3.4E−04 | 0.59 [0.44-0.79] | 0.21 | 0.83 [0.61-1.12] |
| | LumA vs. LumB | 1.8E−08 | 0.38 [0.27-0.53] | 8.1E− | 0.5 [0.35-0.71] |
| | | | | 05 | |

Abbreviations:
PSA: prostate specific antigen,
SMS: positive surgical margin status,
ECE: extracapsular extension,
SVI: seminal vesicle invasion,
LNI: lymph node invasion,
DMFS: distant metastasis-free survival,
PCSS: prostate cancer-specific survival.

TABLE 2
Demographics for pooled retrospective cohort (n = 1,567)

| | | Basal | | Luminal A | | Luminal B | | Total | | P- |
|---|---|---|---|---|---|---|---|---|---|---|
| | | (n = 582) | | (n = 538) | | (n = 447) | | (n = 1567) | | value |
| Age | | 62.4 +/− 6.98 | | 62.5 +/− 6.71 | | 62.4 +/− 6.92 | | 62.4 +/− 6.87 | | 0.975 |
| (years) | | | | | | | | | | |
| | NA | 1 | (0.002) | 1 | (0.002) | 1 | (0.002) | 3 | (0.002) | |
| PSA | <10 | 351 | (0.603) | 318 | (0.591) | 238 | (0.532) | 907 | (0.579) | 0.002 |
| (ng/dL) | 10 to | 131 | (0.225) | 145 | (0.27) | 113 | (0.253) | 389 | (0.248) | |
| | 20 | | | | | | | | | |
| | >20 | 87 | (0.149) | 64 | (0.119) | 92 | (0.206) | 243 | (0.155) | |
| | NA | 13 | (0.022) | 11 | (0.02) | 4 | (0.009) | 28 | (0.018) | |
| Gleason | <6 | 68 | (0.117) | 53 | (0.099) | 23 | (0.051) | 144 | (0.092) | <0.0001 |
| | 7 | 328 | (0.564) | 335 | (0.623) | 218 | (0.488) | 884 | (0.562) | |
| | 8 to | 184 | (0.316) | 149 | (0.277) | 205 | (0.459) | 538 | (0.343) | |
| | 10 | | | | | | | | | |
| | NA | 2 | (0.003) | 1 | (0.002) | 1 | (0.002) | 4 | (0.003) | |
| SM | No | 297 | (0.51) | 261 | (0.485) | 214 | (0.479) | 772 | (0.493) | 0.554 |
| | Yes | 284 | (0.488) | 276 | (0.513) | 232 | (0.519) | 792 | (0.505) | |
| | NA | 1 | (0.002) | 1 | (0.002) | 1 | (0.002) | 3 | (0.002) | |
| SVI | No | 427 | (0.734) | 388 | (0.721) | 300 | (0.671) | 1115 | (0.712) | 0.077 |
| | Yes | 153 | (0.263) | 148 | (0.275) | 145 | (0.324) | 446 | (0.285) | |
| | NA | 2 | (0.003) | 2 | (0.004) | 2 | (0.004) | 6 | (0.004) | |
| ECE | No | 272 | (0.467) | 259 | (0.481) | 147 | (0.329) | 678 | (0.433) | <0.0001 |
| | Yes | 306 | (0.526) | 278 | (0.517) | 298 | (0.667) | 882 | (0.563) | |
| | NA | 4 | (0.007) | 1 | (0.002) | 2 | (0.004) | 7 | (0.004) | |
| LNI | No | 524 | (0.9) | 486 | (0.903) | 383 | (0.857) | 1393 | (0.889) | 0.032 |
| | Yes | 57 | (0.098) | 50 | (0.093) | 63 | (0.141) | 170 | (0.108) | |
| | NA | 1 | (0.002) | 2 | (0.004) | 1 | (0.002) | 4 | (0.003) | |

Abbreviations: PSA: prostate specific antigen, SM: positive surgical margins, SVI: seminal vesicle invasion, ECE: extracapsular extension, LNI: lymph node invasion.

TABLE 3
Univariable and multivariable analysis in pooled retrospective cohort (n = 1,567)

| | | | | MVA | |
|---|---|---|---|---|---|
| | | UVA | | P- | |
| | | P-value | HR [95% CI] | value | HR [95% CI] |
| bRFS | Age (yrs) | 0.36 | 1 [0.99-1.01] | 0.97 | 1 [0.99-1.01] |
| | PSA 10-20 vs. <10 | 0.13 | 1.13 [0.97-1.32] | 0.59 | 1.04 [0.89-1.22] |
| | PSA >20vs. <10 | 8.7E−06 | 1.49 [1.25-1.77] | 0.13 | 1.16 [0.96-1.4] |
| | Gleason 7 vs. <7 | 0.12 | 1.23 [0.95-1.61] | 0.89 | 1.02 [0.77-1.34] |
| | Gleason 8-10 vs. <7 | 3.0E−11 | 2.49 [1.9-3.27] | 3.9E− | 1.83 [1.37-2.43] |
| | | | | 05 | |
| | SM | 1.2E−04 | 1.31 [1.14-1.5] | 0.03 | 1.17 [1.01-1.35] |
| | SVI | 0.0E+00 | 2.01 [1.75-2.31] | 4.5E− | 1.65 [1.41-1.93] |
| | | | | 10 | |
| | ECE | 1.6E−09 | 1.51 [1.32-1.73] | 0.10 | 1.13 [0.98-1.31] |
| | LNI | 4.3E−05 | 1.49 [1.23-1.8] | 0.21 | 0.87 [0.71-1.08] |
| | Basal vs. LumB | 3.2E−06 | 0.69 [0.59-0.81] | 0.01 | 0.81 [0.69-0.96] |
| | LumA vs. LumB | 5.4E−07 | 0.66 [0.57-0.78] | 4.8E− | 0.79 [0.66-0.93] |
| | | | | 03 | |
| OS | Age (yrs) | 7.6E−05 | 1.03 [1.01-1.04] | 0.01 | 1.02 [1-1.03] |
| | PSA 10-20 vs. <10 | 0.62 | 1.06 [0.85-1.31] | 0.20 | 0.87 [0.7-1.08] |
| | PSA >20vs. <10 | 0.01 | 1.35 [1.08-1.69] | 0.15 | 0.83 [0.65-1.07] |
| | Gleason 7 vs. <7 | 1.1E−03 | 1.98 [1.32-2.98] | 0.02 | 1.69 [1.11-2.59] |
| | Gleason 8-10 vs. <7 | 1.1E−14 | 5.01 [3.33-7.53] | 5.2E− | 3.65 [2.36-5.63] |
| | | | | 09 | |
| | SM | 2.4E−03 | 1.32 [1.1-1.59] | 0.06 | 1.2 [1-1.45] |
| | SVI | 1.2E−13 | 1.96 [1.64-2.33] | 1.7E− | 1.49 [1.21-1.83] |
| | | | | 04 | |
| | ECE | 1.2E−07 | 1.66 [1.38-2.01] | 0.34 | 1.11 [0.9-1.37] |
| | LNI | 6.0E−10 | 2.1 [1.66-2.65] | 0.03 | 1.33 [1.02-1.72] |
| | Basal vs. LumB | 5.1E−04 | 0.69 [0.56-0.85] | 0.24 | 0.88 [0.71-1.09] |
| | LumA vs. LumB | 2.8E−07 | 0.56 [0.45-0.7] | 1.9E− | 0.69 [0.55-0.87] |
| | | | | 03 | |

Abbreviations:
PSA: prostate specific antigen,
SM: positive surgical margins,
SVI: seminal vesicle invasion,
ECE: extracapsular extension,
LNI: lymph node invasion,
bRFS: biochemical recurrence-free survival,
OS: overall survival.

TABLE 4
Univariable and multivariable analysis in pooled retrospective cohort (n = 1,567) to
examine independence of subtypes from D'Amico Risk Classification

| | | UVA | | MVA | |
|---|---|---|---|---|---|
| | | P-value | HR 1195% CI | P-value | HR 1195% CI |
| bRFS | Age | 0.36 | 1 [0.99-1.01] | 0.30 | 1.01 [0.99-1.02] |
| | D'Amico | 3.49E−10 | 1.87 [1.54-2.27] | 5.71E−08 | 1.73 [1.42-2.1] |
| | LNI | 4.32E−05 | 1.49 [1.23-1.8] | 2.66E−15 | 3.46 [2.55-4.71] |
| | Basal vs. LumB | 3.23E−06 | 0.69 [0.59-0.81] | 0.01 | 0.78 [0.65-0.93] |
| | LumA vs. LumB | 5.44E−07 | 0.66 [0.57-0.78] | 1.40E−03 | 0.74 [0.61-0.89] |
| DMFS | Age | 0.88 | 1 [0.99-1.02] | 0.30 | 1.01 [0.99-1.03] |
| | D'Amico | 3.71E−10 | 2.86 [2.06-3.97] | 7.89E−08 | 2.48 [1.78-3.46] |
| | LNI | 0.00 | 2.56 [2.06-3.19] | 6.78E−10 | 3.21 [2.22-4.65] |
| | Basal vs. LumB | 8.95E−11 | 0.5 [0.4-0.61] | 2.19E−06 | 0.54 [0.42-0.7] |
| | LumA vs. LumB | 9.14E−14 | 0.42 [0.34-0.53] | 8.53E−08 | 0.49 [0.38-0.64] |
| PCSS | Age | 0.86 | 1 [0.98-1.02] | 0.31 | 1.01 [0.99-1.04] |
| | D'Amico | 7.47E−06 | 3 [1.86-4.86] | 6.06E−05 | 2.7 [1.66-4.4] |
| | LNI | 2.22E−15 | 3.19 [2.4-4.25] | 5.72E−04 | 2.71 [1.54-4.78] |
| | Basal vs. LumB | 3.40E−04 | 0.59 [0.44-0.79] | 0.06 | 0.71 [0.5-1.01] |
| | LumA vs. LumB | 1.84E−08 | 0.38 [0.27-0.53] | 7.95E−05 | 0.45 [0.3-0.67] |
| OS | Age | 7.62E−05 | 1.03 [1.01-1.04] | 1.09E−05 | 1.04 [1.02-1.06] |
| | D'Amico | 2.63E−06 | 1.98 [1.49-2.63] | 3.12E−05 | 1.85 [1.38-2.46] |
| | LNI | 6.02E−10 | 2.1 [1.66-2.65] | 2.28E−03 | 2.22 [1.33-3.71] |
| | Basal vs. LumB | 5.09E−04 | 0.69 [0.56-0.85] | 0.17 | 0.84 [0.66-1.08] |
| | LumA vs. LumB | 2.77E−07 | 0.56 [0.45-0.7] | 4.23E−05 | 0.57 [0.43-0.74] |

Abbreviations:
LNI: lymph node invasion.
Note:
D'Amico high-risk was compared to intermediate and low risk combined, as there were only 19 low risk patients.

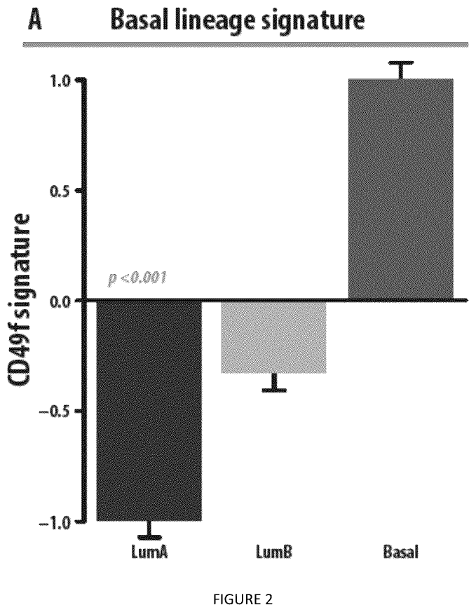

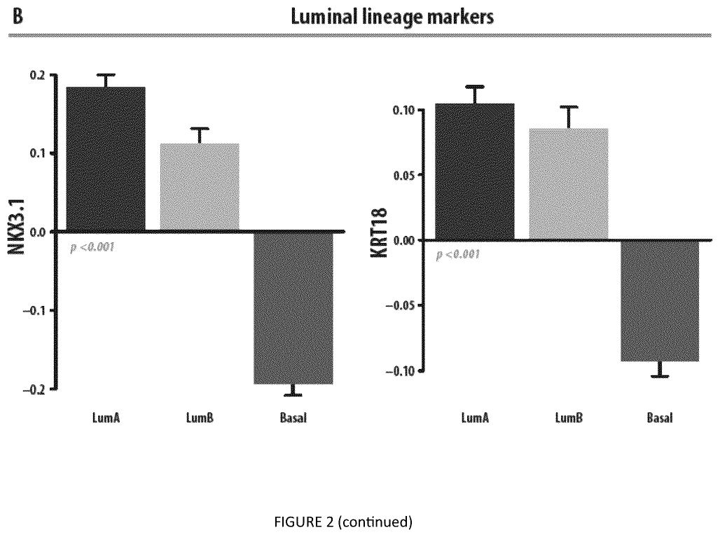

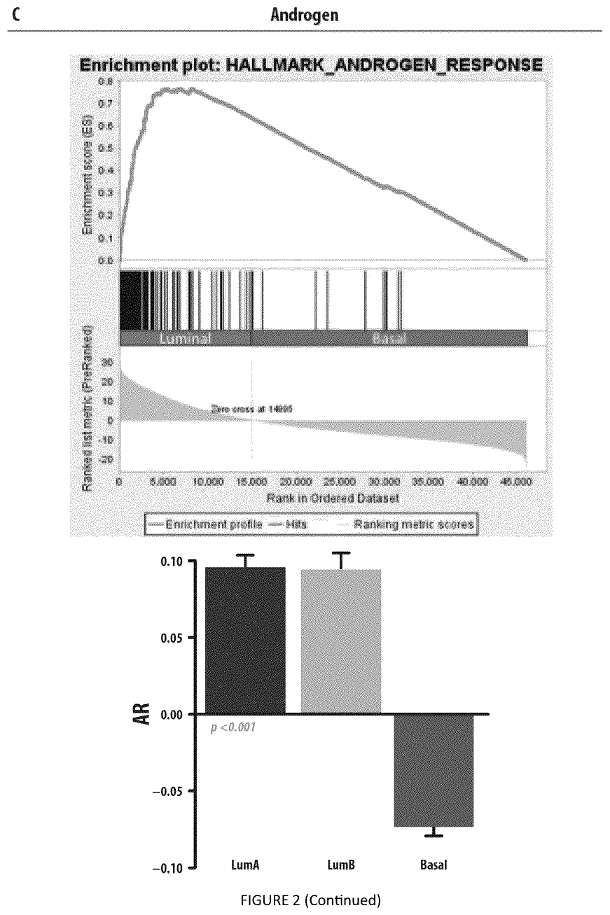

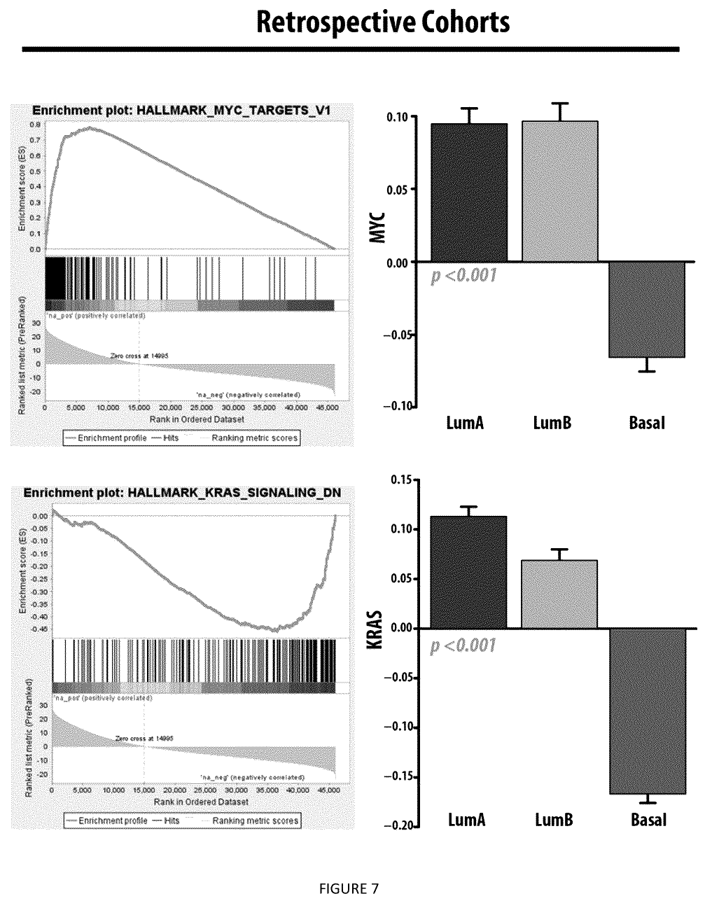

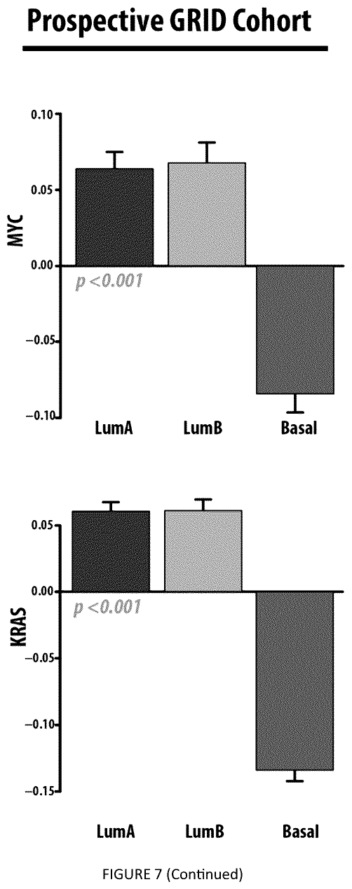

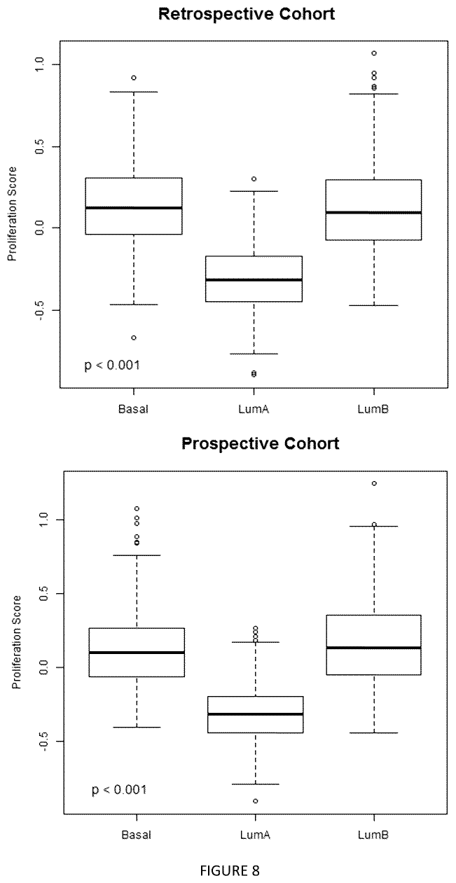

We then investigated the relationship between these subtypes and luminal and basal prostate cancer lineage markers. The basal lineage CD49f signature (Smith et al. Proc Natl Acad Sci USA. 2015;112 (47): E6544-6552) is increased in basal-like samples (ANOVA p<0.001, FIG. 2A). Concordantly, the luminal markers NKX3.1, KRT18, and AR are increased in luminal-like samples (ANOVA p<0.001, FIG. 2B-C). Consistent with our findings for AR, the androgen activity pathway is enriched in luminal compared to basal (ANOVA p<0.001, FIG. 2C). Examining the top GSEA hallmark concepts comparing luminal to basal (Supplementary Methods) reveals that the MYC pathway is the top enriched pathway in luminal-like samples, and genes down-regulated by KRAS are the top positive pathway in basal-like samples (negatively enriched in luminal samples). These results are concordant with MYC and KRAS expression, which are both increased in luminal-like samples (ANOVA p<0.001, FIG. 7). Upon observing that proliferation genes such as MKI67 are low in luminal A (FIG. 1A), we formally examined the subtypes using the PAM50 proliferation score (Parker et al. J Clin Oncol. 2009; 27 (8): 1160-1167). Luminal A has a lower proliferation score than luminal B and basal (p<0.001, FIG. 8), which may explain the divergent clinical outcomes despite the biological similarities between luminal A and B.

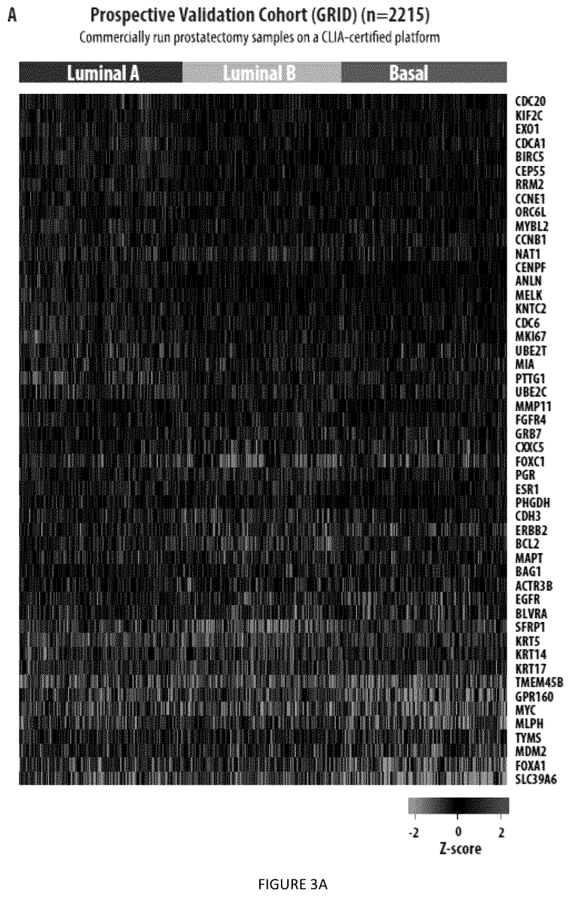

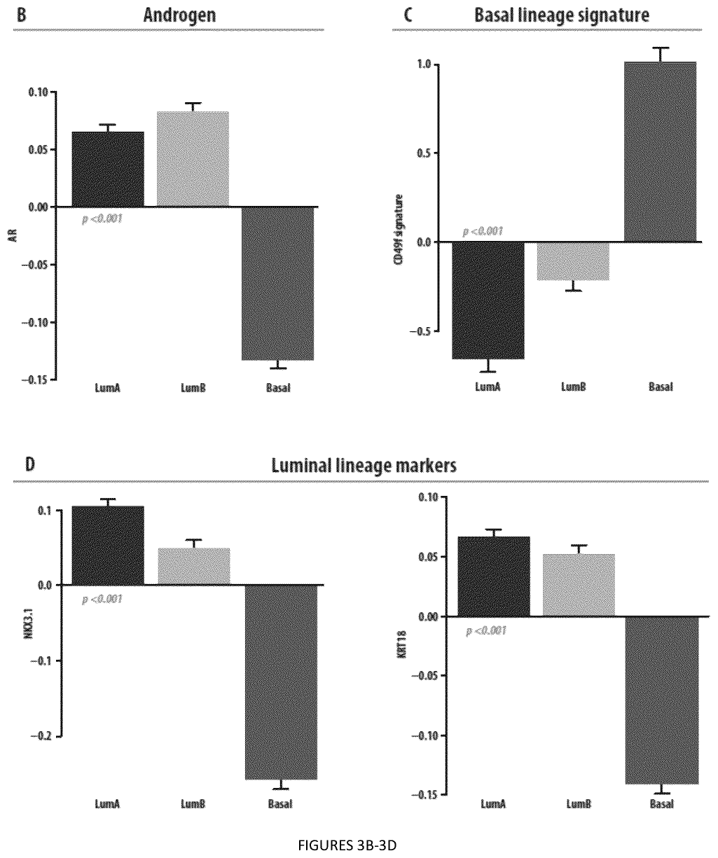

We next independently validated the associations of these subtypes with biologic and clinicopathologic factors in Decipher GRID™, a prospectively-collected cohort of 2,215 prostatectomy patient expression profiles. PAM50 gene expression patterns are similar to the pooled retrospective cohorts, and trends of AR/AR-signaling (higher in luminal), CD49f signature (higher in basal), and NKX3.1 and KRT19 (both higher in luminal) gene expression are conserved (FIGS. 3A-D). 33.3% of samples are classified as luminal A, 32.6% as luminal B, and 34.1% as basal. We also confirmed MYC and KRAS expression patterns, which are both increased in luminal samples (FIG. 7). Finally, while Decipher GRID does not have associated clinical outcomes, luminal B demonstrates the highest Gleason scores, as well as rates of SVI, ECE, and LNI, consistent with clinical outcomes and clinicopathologic data in our retrospective cohorts (Table 5).

TABLE 5
Demographics for GRID (n = 2,215)

| | | Basal | | Luminal A | | Luminal B | | Total | | |
|---|---|---|---|---|---|---|---|---|---|---|
| | | (n = 755) | | (n = 737) | | (723) | | (n = 2215) | | P-value |
| Age | | 64.1 +/− 7.09 | | 64.4 +/− 6.64 | | 64.9 +/− 6.61 | | 64.4 +/− 6.79 | | 0.109 |
| (years) | | | | | | | | | | |
| | NA | 67 | (0.089) | 63 | (0.085) | 59 | (0.082) | 189 | (0.085) | |
| PSA | <10 | 382 | (0.506) | 373 | (0.506) | 360 | (0.498) | 1115 | (0.503) | 0.439 |
| (ng/dL) | 10 to | 72 | (0.095) | 85 | (0.115) | 90 | (0.124) | 247 | (0.112) | |
| | 20 | | | | | | | | | |
| | >20 | 33 | (0.044) | 28 | (0.038) | 36 | (0.05) | 97 | (0.044) | |
| | NA | 268 | (0.355) | 251 | (0.341) | 237 | (0.328) | 756 | (0.341) | |
| Gleason | <6 | 61 | (0.081) | 53 | (0.072) | 29 | (0.04) | 143 | (0.065) | <0.0001 |
| | 7 | 453 | (0.6) | 489 | (0.664) | 431 | (0.596) | 1373 | (0.62) | |
| | 8 to | 174 | (0.23) | 132 | (0.179) | 204 | (0.282) | 510 | (0.23) | |
| | 10 | | | | | | | | | |
| | NA | 67 | (0.089) | 63 | (0.085) | 59 | (0.082) | 189 | (0.085) | |
| SM | No | 310 | (0.411) | 295 | (0.4) | 299 | (0.414) | 904 | (0.408) | 0.878 |
| | Yes | 375 | (0.497) | 376 | (0.51) | 365 | (0.505) | 1116 | (0.504) | |
| | NA | 70 | (0.093) | 66 | (0.09) | 59 | (0.082) | 195 | (0.088) | |
| SVI | No | 530 | (0.702) | 554 | (0.752) | 499 | (0.69) | 1583 | (0.715) | 0.003 |
| | Yes | 152 | (0.201) | 116 | (0.157) | 165 | (0.228) | 433 | (0.195) | |
| | NA | 73 | (0.097) | 67 | (0.091) | 59 | (0.082) | 199 | (0.09) | |
| ECE | No | 336 | (0.445) | 325 | (0.441) | 260 | (0.36) | 921 | (0.416) | <0.0001 |
| | Yes | 348 | (0.461) | 345 | (0.468) | 403 | (0.557) | 1096 | (0.495) | |
| | NA | 71 | (0.094) | 67 | (0.091) | 60 | (0.083) | 198 | (0.089) | |
| LNI | No | 646 | (0.856) | 628 | (0.852) | 624 | (0.863) | 1898 | (0.857) | 0.015 |
| | Yes | 8 | (0.011) | 9 | (0.012) | 21 | (0.029) | 38 | (0.017) | |
| | NA | 101 | (0.134) | 100 | (0.136) | 78 | (0.108) | 279 | (0.126) | |

Abbreviations: PSA: prostate specific antigen, SM: positive surgical margins, SVI: seminal vesicle invasion, ECE: extracapsular extension, LNI: lymph node invasion.

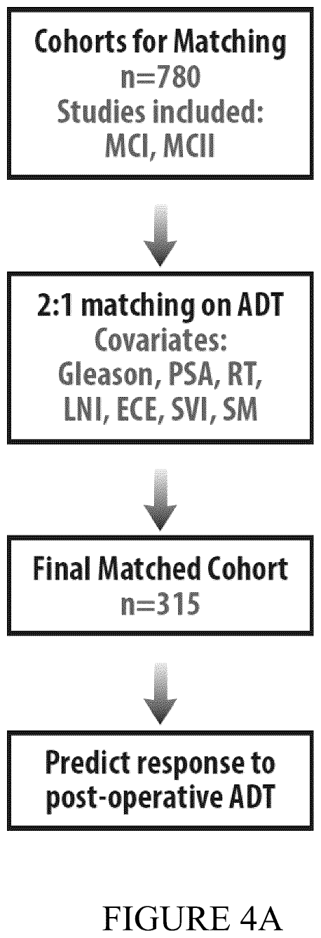

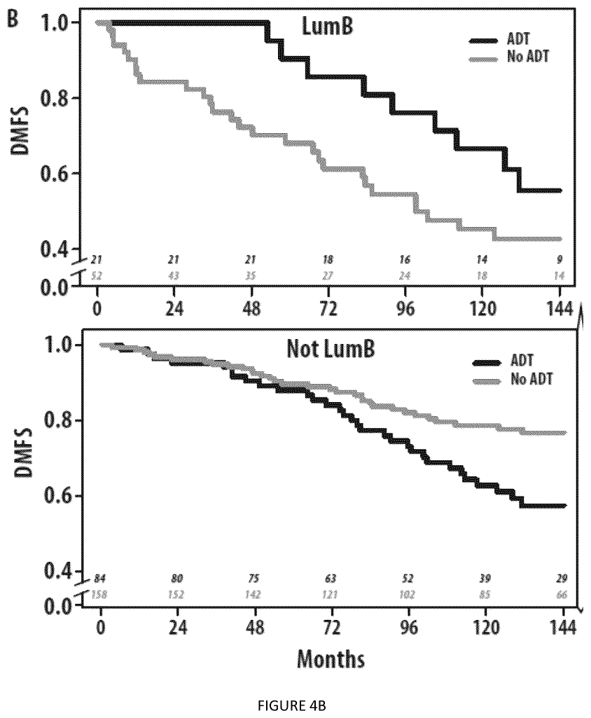

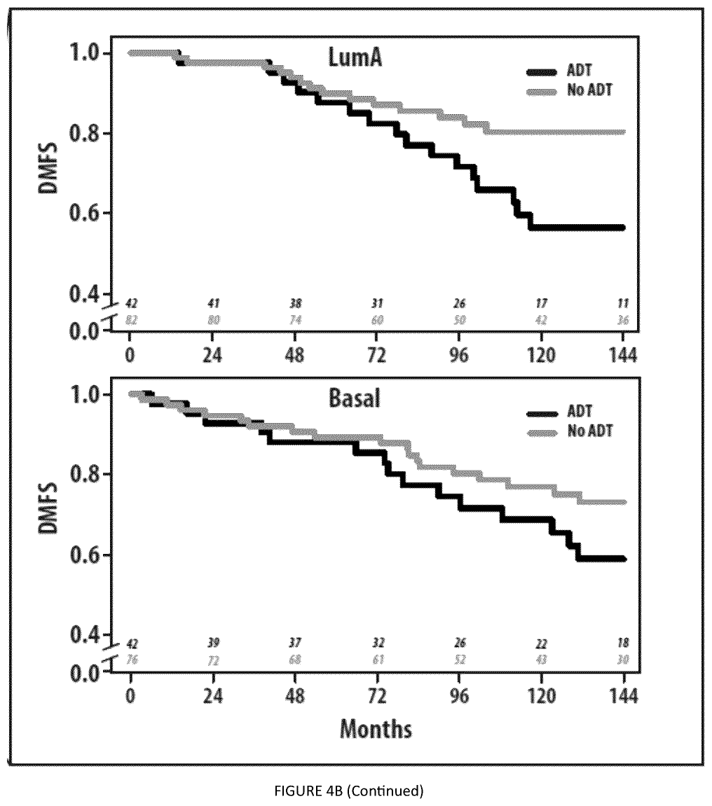

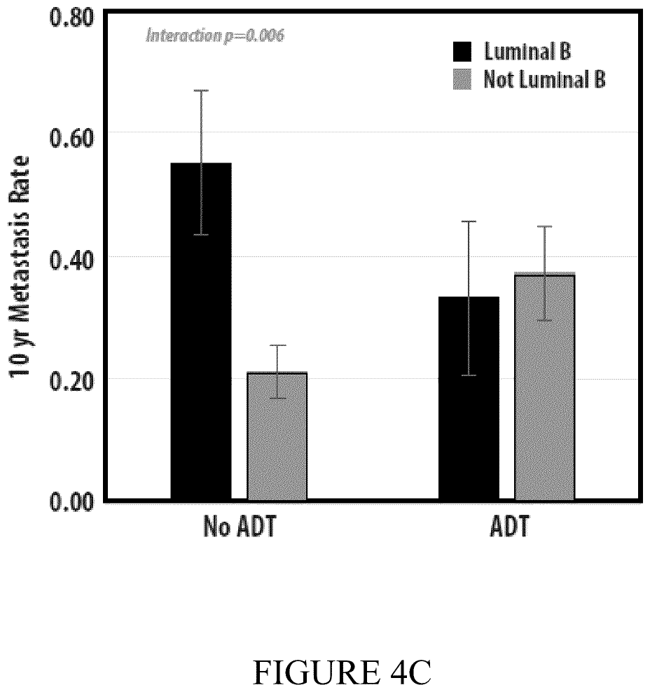

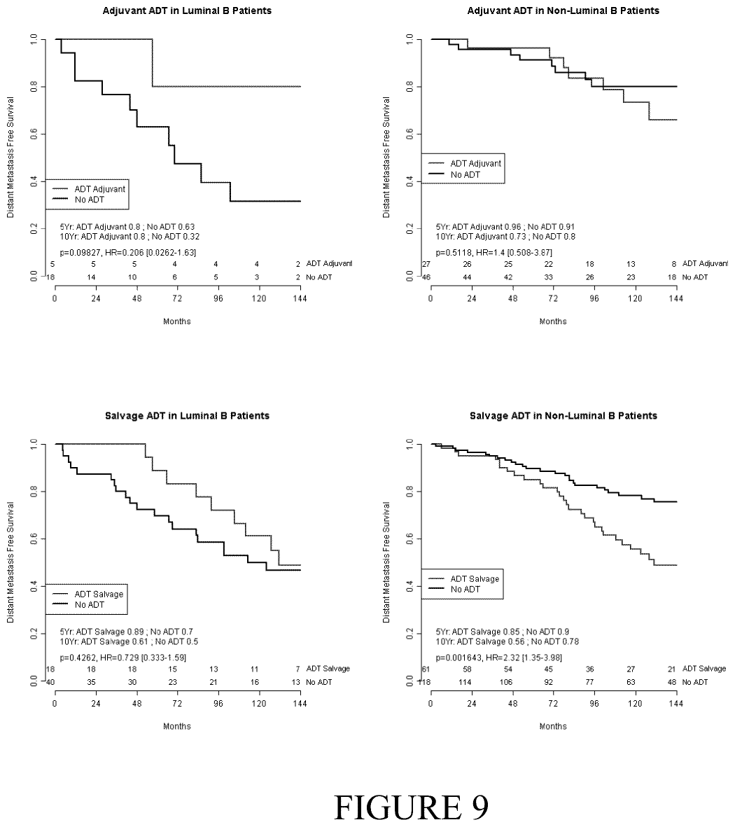

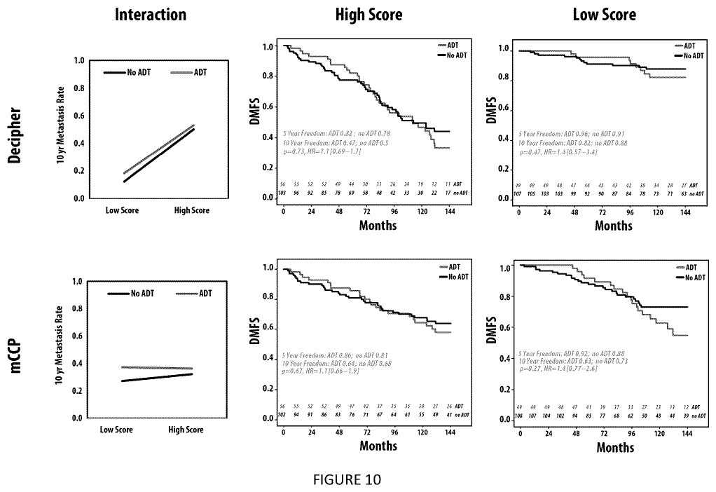

The relationship between androgen signaling and luminal-like prostate cancer is of particular interest given the importance of ADT in treating prostate cancer. We investigated whether these subtypes could predict response to hormonal therapy in an exploratory subgroup analysis by first designing a post-prostatectomy ADT treated/untreated sub-cohort (n=315; ADT untreated n=210; ADT treated n=105) matched by clinicopathologic factors (Gleason, PSA, LNI, ECE, SVI, and SM), and post-operative RT (FIG. 4A and Tables 6 and 7). Matching was performed with a 2:1 ratio of patients (ADT untreated:ADT treated). The matched cohort had a median follow-up time of 13 years. In this analysis, we pool the luminal A and basal subtypes to compare to luminal B, as the luminal A and basal subtypes have similar outcomes for ADT and no ADT in the matched cohorts. In the luminal B subtype, which has the worst prognosis of the three subtypes and contains patients with increased expression of AR signaling genes, patients treated with ADT have improved DMFS compared to those that did not receive ADT (10-year metastasis rates of 33% vs. 55%, FIGS. 4B, 4C). However, in the non-luminal B patients, patients treated with ADT have worse DMFS compared to untreated patients (10-year metastasis rates of 37% vs. 21%, FIGS. 4B, 4C), with a similar trend in both luminal A and basal patients. Separating patients receiving adjuvant or salvage therapy in the matched cohort results in similar trends, although the p-values are insignificant due to the reduced numbers (FIG. 9). Finally, we used interaction analysis in a Cox model of these matched patients to demonstrate a statistically significant interaction term between ADT and the luminal B subtype (p=0.006). Luminal B represents a subgroup of prostate cancers with poor prognosis combined with biological differences in AR signaling that result in improved response to post-operative ADT.

TABLE 6
Demographics for matched cohort (n = 315)

| | | Basal | | Luminal A | | Luminal B | | Total | | P- |
|---|---|---|---|---|---|---|---|---|---|---|
| | | (n = 118) | | (n = 124) | | (n = 73) | | (n = 315) | | value |
| Age | | 64.4 +/− 6.64 | | 64.2 +/− 7.04 | | 64.8 +/− 6.65 | | 64.4 +/− 6.79 | | 0.860 |
| (years) | | | | | | | | | | |
| PSA | <10 | 75 | (0.636) | 80 | (0.645) | 37 | (0.507) | 192 | (0.61) | 0.273 |
| (ng/dL) | 10 to | 19 | (0.161) | 24 | (0.194) | 17 | (0.233) | 60 | (0.19) | |
| | 20 | | | | | | | | | |
| | >20 | 24 | (0.203) | 20 | (0.161) | 19 | (0.26) | 63 | (0.2) | |
| Gleason | <6 | 13 | (0.11) | 7 | (0.056) | 1 | (0.014) | 21 | (0.067) | 0.002 |
| | 7 | 72 | (0.61) | 91 | (0.734) | 41 | (0.562) | 204 | (0.648) | |
| | 8 to | 33 | (0.28) | 26 | (0.21) | 31 | (0.425) | 90 | (0.286) | |
| | 10 | | | | | | | | | |
| SM | No | 57 | (0.483) | 49 | (0.395) | 32 | (0.438) | 138 | (0.438) | 0.387 |
| | Yes | 61 | (0.517) | 75 | (0.605) | 41 | (0.562) | 177 | (0.562) | |
| SVI | No | 105 | (0.89) | 100 | (0.806) | 62 | (0.849) | 267 | (0.848) | 0.196 |
| | Yes | 13 | (0.11) | 24 | (0.194) | 11 | (0.151) | 48 | (0.152) | |
| ECE | No | 70 | (0.593) | 79 | (0.637) | 34 | (0.466) | 183 | (0.581) | 0.059 |
| | Yes | 48 | (0.407) | 45 | (0.363) | 39 | (0.534) | 132 | (0.419) | |
| LNI | No | 118 | (1) | 124 | (1) | 73 | (1) | 315 | (1) | NA |

Abbreviations: PSA: prostate specific antigen, SM: positive surgical margins, SVI: seminal vesicle invasion, ECE: extracapsular extension, LNI: lymph node invasion.

TABLE 7
Number of patients receiving ADT and RT in the
matched cohort (n = 315)

| | Adjuvant | Salvage | Both | |
|---|---|---|---|---|
| | ADT | ADT | Adjuvant and | No |
| | Only | Only | Salvage ADT | ADT |
| Adjuvant RT | 4 | 6 | 2 | 23 |
| Only | | | | |
| Salvage RT Only | 3 | 14 | 2 | 41 |
| Both Adjuvant | 1 | 0 | 0 | 0 |
| and Salvage RT | | | | |
| No RT | 18 | 53 | 2 | 146 |

Abbreviations:
ADT: Androgen deprivation therapy;
RT: Radiation therapy

These results showed that a genomic classifier of the present invention could be utilized to identify three subtypes in prostate cancer subjects. These results suggested that the methods and markers of the present invention would be useful for diagnosing, prognosing, determining the progression of cancer, or predicting benefit from therapy in a subject having prostate cancer. These results further showed that the subtyping methods and genomic classifiers of the present invention are useful for predicting benefit from androgen deprivation therapy (ADT) and treating a subject with prostate cancer. The results showed that the subtyping methods of the present invention may be used to determine a treatment for a subject with prostate cancer.

TABLE 8
Subtyping Target Sequences

| | | Affy | |
|---|---|---|---|
| SEQ | | Probeset | |
| ID NO. | Gene | ID | Sequence |
| 1 | CDC20 | 2333138 | GGGTGCTAGGCCGGAAGGGGCTGCAGCCGAGGGTGGC |
| | | | CCTGATTTTGTGGCCGGCCAGGAGCGAAGGGGTCCCTT |
| | | | TCTGTCCCCTGAGCACCGTCGCCTCCT |
| 2 | CDC20 | 2333139 | GCACCAACTGCAAGGACCCCTCCCCCTGCGGGC |
| 3 | CDC20 | 2333140 | TTCGCGTTCGAGAGTGACCTGCACTCGCTGCTTCAGCTG |
| | | | GATGCACCCATCCCCAATGCACCCCCTGCGCGCTGGCA |
| | | | GCGCAAAGCCAAGGAAGCCGCAGGCCCGGCCCCCTCAC |
| | | | CCATGCGGGCCGCCAACCGATCCCACAGCGC |
| 4 | CDC20 | 2333142 | GCAAATCCAGTTCCAAGGTTCAGACCACTCCTAGCAAA |
| | | | CCTGGCGGTGACCGCTATATCCCCCATCGCAGTGCTGCC |
| | | | CAGATGGAGGTGGCCAGCTTCCTCCTGAGCAAGGAGAA |
| | | | CCAGCCTGAAAACAGCCAGACGCCCACCA |
| 5 | CDC20 | 2333144 | GTAGAGGAAGCCAAGATCCTTCGGCTCAGTGGAAAACC |
| | | | ACAAAATGCGCCAGAGG |
| 6 | CDC20 | 2333145 | CGAAGTTCCTGGTTCCTGGAGGGAG |
| 7 | CDC20 | 2333146 | CCTCTTCCTATCTAAGATTGAGGGCAAG |
| 8 | CDC20 | 2333147 | CAGAACAGACTGAAAGTACTCTACAGCCAA |
| 9 | CDC20 | 2333148 | CCTGCCAGACCGTATCCTGGATGCGCCTGAAATCC |
| 10 | CDC20 | 2333149 | ACCTGAACCTTGTGGATTGGAGTTCTGGGAATGTACTG |
| | | | GCCGTGGCACTGGACAACAGTGTGTACCTGTGGAGTGC |
| | | | AAGCTCTGGTGACATCCTGCAGCTTTTGCAAATGGAGC |
| | | | AGCCTGGGGAATATATATCCTCTGTGGCCTG |
| 11 | CDC20 | 2333150 | CAGCAGCAGAAACGGCTTCGAAATATGACCAGTCACTC |
| | | | TGCCCGAGTGGGCTCCCTAAGCTGGAACAGCTATAT |
| 12 | CDC20 | 2333151 | CATCCACCACCATGATGTTCGGGTAGCAGAACACCATG |
| | | | TGGCCACACTGAGTGGCCACAGCCAGGAAGTGTGTGGG |
| | | | CTGCGCTGGGCCCCAGATGGACGACATTTGGCCAGTGG |
| | | | T |
| 13 | CDC20 | 2333152 | GTAATGATAACTTGGTCAATGTGTGGCCTAGTGCTCCTG |
| | | | GAGAGGGTGGCTGGGTTCCTCTGCAGACATTCACCCAG |
| | | | CATCAAGGGGCTGTCAA |
| 14 | CDC20 | 2333155 | GTGTCCCTGGCAGTCCAATGTCCTGGCAACA |
| 15 | CDC20 | 2333156 | GCCTGTCTGAGTGCCGTGGATGCCC |
| 16 | CDC20 | 2333158 | TTATTTGGAAGTACCCAACCATGGC |
| 17 | CDC20 | 2333159 | CACACATCCCGGGTCCTGAGTCTGACCATGAGCCCAGA |
| | | | TGGGGCCACAGTGGCATCCGCAGCAGCAGATGAGACCC |
| | | | TGAGGCTATGGCGCTGTTTTGAGTTGGACCC |
| 18 | CDC20 | 2333160 | AGACCAACCCATCACCTCAGTTGTTTTTTAT |
| 19 | KIF2C | 2334099 | TGCGGCGGTTTACGCGGCGTTAAGACTTCGTAGGGTTA |
| | | | GCGAAATTGAGGTTTCTTGGTATTGCGCGTTTCTCTT |
| 20 | KIF2C | 2334101 | GTCCCTAGGTCAAGGGGACTCGTGA |
| 21 | KIF2C | 2334103 | GTTTAATTCACAGTGCCAATGTAAGGACTGT |
| 22 | KIF2C | 2334105 | TCTTAGGGTTAGGTAGCAGCTGTCAGGAACTTGCCCCT |
| | | | GCCCATAAGATCCTAAAGGGCCCCCATTTGACTCTCAC |
| | | | CAGACAGTTAGAACTTGTTTCCTCCTCCGTGTCAGCCAT |
| | | | CAAGAGGTGCTTGGGGGGCTGTGCCCAGCAGGACCTCA |
| | | | CTGCCCAGCAGATCAGCAGGGGAGCCAAGTGGCCTAGA |
| | | | TCTGCTGTGGAGTACCCGACTGTTTGCCTGCCTGTCTGC |
| | | | CCTCCTCTTCACCTCATTCTCATCACTGACGTCTACCATT |
| | | | GGCTT |
| 23 | KIF2C | 2334106 | ATGATGTGGCTGCAATAAACCCAGAACTCTTACAGCTT |
| | | | CTTCCCTTACATCCGAAGGACAATCTGCCCTTGC |
| 24 | KIF2C | 2334107 | CAAAAACGGAGATCCGTCAACTCCAAAATTCC |
| 25 | KIF2C | 2334109 | GGACCTATTTCACCTTGTACAGAAACTTGGAGGTTTGCC |
| | | | CCTGACCACCCTCGAGATCGTGCAGCACTGACTGGCTA |
| | | | CTGCTCTCGGTTCTCCA |
| 26 | KIF2C | 2334111 | CTGCCCCCACTAGGCCTTCCTGCCCTGCAG |
| 27 | KIF2C | 2334112 | GGCTGAAATACCATTGAGGATGGTCAGCGAGGAGATGG |
| | | | AAGAGCAAGTCCATTCCATCCGAGGC |
| 28 | KIF2C | 2334113 | AGGCCCAGAACTCTGAAATGAGAATGAA |
| 29 | KIF2C | 2334114 | TATGACAGTAGTTTTCCAAACTGGGAATTTGCCCGAAT |
| | | | GATTAAAGAATTTCGGGCTACTTTGGAATGTCATCCACT |
| | | | TACTATGACTGATCC |
| 30 | KIF2C | 2334117 | GAGCACCCCCTGAAATACTCTCCTTC |
| 31 | KIF2C | 2334118 | ACATGAACCCAAGTTGAAAGTGGACTTAACAAAGTATC |
| | | | TGGAGAACCAAGCATTCTGCTTTGACTTTGCATTTGATG |
| | | | AAACAGCTTCG |
| 32 | KIF2C | 2334119 | AGGCCACTGGTACAGACAATCTTTGAAGGTGGA |
| 33 | KIF2C | 2334122 | CGGGACGTCTTCCTCCTGAAGAATCAACCCTGCTACCG |
| | | | GAAGTTGGGCCTGGAAGTCTATGTGACATTCTTCGAGA |
| | | | TCTACAATGGGA |
| 34 | KIF2C | 2334125 | AGGACGGCAAGCAACAGGTGCAAGTGGTGGGGCTGCA |
| | | | GGAGCATCTGGTTAACTCTGCTGATGATGTCATCAAGA |
| | | | TGATCGACATGGGCAGC |
| 35 | KIF2C | 2334126 | GCTCCCACGCGTGCTTCCAAATTATTCTTCGAGCTAAAG |
| | | | GGAGAATGCATGGCAAGTTCTCTTTGGTAGATCTGGCA |
| | | | GGGAATGAGCGAGGCGCGGACACTTCCAGTGCTGACCG |
| | | | GCAGACCCGCATGGAGGGCGCAGAAATCAACAAGAGT |
| | | | CTC |
| 36 | KIF2C | 2334130 | GCTGGGTGAGGGGCTTTTCCAGTCCA |
| 37 | KIF2C | 2334131 | GCAAGGTTGCCATTCCATCCCCTTGGAGCCTCAAGCCTC |
| | | | GAAGCCTGGGCGGTGCCACATTCCTC |
| 38 | KIF2C | 2334132 | AGTACCAAGGGGGTGCTGTGGGATCTGAGACCTCCTTG |
| | | | TTTCCT |
| 39 | KIF2C | 2334133 | GAAGAGATGGAAGCCTGCTCTAACGGGGCGCTGATTCC |
| | | | AG |
| 40 | KIF2C | 2334134 | CCAGATGTCCAGCTTTAACGAAGCCATGACTCAGA |
| 41 | KIF2C | 2334135 | AAGGACCAGACTGGCTTGAGCTCTCTGAGATGACCGAG |
| | | | CAGCCAGACTATGACCTGGAGACCTTTGTGAACAAAGC |
| | | | GGAATCTGCTCTGGCCCAGC |
| 42 | KIF2C | 2334136 | GTCATCAAGGCCTTGCGCCTGGCCATG |
| 43 | KIF2C | 2334137 | CGACTGCAAATAAAAATCTGTTTGGTTTG |
| 44 | KIF2C | 2334138 | GGGACAGGTTCTGGTAAATGCCAAGTATG |
| 45 | KIF2C | 2334139 | TTCCTCAGTTGTCGCCCTCACGAGAGGAAGGAGCTCTT |
| | | | AGTTACCCTTTTGTGTTGCCCTTCTTTCCATCAAGGGGA |
| | | | ATGTTCTCAGCATAGAGCTTTCTCCGCAGCATCCTGCCT |
| | | | GCGTGGACTGGCTGCTAATGGAGAGCTCCCTGGGGTTG |
| | | | TCCTGGCTCTGGGGAGAGAGACGGAGCCTTTAGTACAG |
| | | | CTATCTGCTGGCTCTAAACCTTCTACGCCTTTGGGCCGA |
| | | | GCACTGAATGTCTT |
| 46 | KIF2C | 2334140 | GTCTCCCTAGAGATCCTAGAGGATCCCTACTGTTTTC |
| 47 | PHGDH | 2354635 | AGGAGCCACATGCTCTCATCAAGCAGAAA |
| 48 | PHGDH | 2354639 | GTGCCCAACCAGTGAGGCCACGTTTCGAAAAGAAGAAA |
| | | | GAAACGACAAACTAAAATACATGACTGTGTAGATGAGG |
| 49 | PHGDH | 2354641 | GCTGGAGAATACTGCCCAGTTACTCTAGCGCGCCAGGC |
| | | | CGAACCGCAGCTTCTTGGCTTAGGTACTTCTACTCACAG |
| | | | CGGCCGA |
| 50 | PHGDH | 2354642 | ATGGCTTTTGCAAATCTGCGGAAAGTGCTCATCAGTGA |
| | | | CAGCCTGGACCCTTGCTGCCGGAAGATCT |
| 51 | PHGDH | 2354644 | GCAGCCTAAGCATCATTCCTCTTCTCTTCTTAGTGGAGA |
| | | | TAAAATTACCCACTGCTCTCCTTACATT |
| 52 | PHGDH | 2354645 | GTGTGGGCCCTTACCCTAGAAGCCAACTTCTCATGACCT |
| | | | TTCTCTATCTCCAGAATCCATGCAGTGGGAATGAAGGT |
| | | | AAAAGAAGGTTTTCATGGGATCCAGCTGAGAGCTCTAC |
| | | | GGGGAAAATGGATCTGAGGAGCCATGTGCTCCATCTCT |
| | | | TTTATTTTACAGGTAGAGACTAGGGGTATAGAGTGAGG |
| | | | TGAATTACCGCAGTGACCCACACATTGTTGGCAGACCT |
| | | | AGGATTAGAACTCTGTCTTCCTGGTTCCCAGCTTGGTGC |
| | | | TTTTGAAAGCATACTTGCTGCTTTCTTACCGGCCTGGTG |
| | | | TCTGCCACTTTGGGACAGAGTGTGGACTTGCTCACCTGC |
| | | | CCCATTTCTTAGGGATTCTCATTCTGTGTTTGAGCAAGA |
| | | | ATATTCTTATTCTGGAAAGAACCACATACCACAGGATT |
| | | | CTGGGTGAGCATAAGGAAGATTGTCTTGGGGATCTGAC |
| | | | TTAGCTCACGTATAGTGGCTATGATGAATTCAGTGTCTT |
| | | | ATTTTTTGCATATGTATATTTTTAGTCTAATATTGCCTGG |
| | | | GTGTCTGAGCAAGTCTAGATGAATTTAATTGCTCTCATT |
| | | | TTTCCCCTGCCCCTCTTCCTTTGGTCTCTCTTTTAGGAAA |
| | | | TGTTTTTCTTTCAACATTCGTTTCATTCATTATTTACTCA |
| | | | TTCGGCCAACCAACATTTATTGAGTGCCTTCCCTGTATC |
| | | | AGGGACAGGGGCTTACAAAGTAGAATTTGATCCCACCT |
| | | | CTGCCCTCAGTAGCTCAGTGTCTAATGGAGGTAGTGAT |
| | | | GTTCATTAAGCGTCGCCAGATACTGTGCTAGGTGCTGTG |
| | | | CCTGTTCTCTCTCGCTTGTTCCTCACACACTTGAGAAGG |
| | | | CCGAAGCTGATTCATAGCTTGGAAGGCAGGGGCCTTGG |
| | | | ATTTGAACCCAGGCCTGACCAATGGCAGAACCTATCAG |
| | | | ATGTGTGGACAGATGACATTGCCTTTCTTTCTTTGGATA |
| | | | TATCAAAATCAGCCAGCAGGCAGGAACTCCCATTTT |
| 53 | PHGDH | 2354646 | TGTGTCTGATGGACATCCAGGCTGCAGG |
| 54 | PHGDH | 2354647 | ATGGTGCTGTCTAGAGAGATGAGCCAGGTGCCCAGAGC |
| | | | CCATGGGCCAATGCTGCCCTTTCTTGAGCATGCCAAAC |
| | | | AAAGCGGTTG |
| 55 | PHGDH | 2354648 | CAGTCTCCTCCACTCTAAGTAAAAATCAGCATGAGTCCT |
| | | | AGCCCACATTT |
| 56 | PHGDH | 2354649 | TGAGTATACCAAAGATATCTATGAACTGGCAGTCATCA |
| | | | GTGACTTCCTAAGGTTCCGGAAATGCATCTCTT |
| 57 | PHGDH | 2354650 | ATGTGCCTGCGGCTTTACGAGTTCTCACAGAATGACTTT |
| | | | C |
| 58 | PHGDH | 2354651 | ACTGTGAAGGCCTTATTGTTCGCTCTGCCACCAAGGTGA |
| | | | CCGCTGATGTCATCAACGCAGCTGAGAAACTCC |
| 59 | PHGDH | 2354652 | ATCTGGAGGCCGCAACAAGGAAGGGCATCTT |
| 60 | PHGDH | 2354653 | TGAGTGCGGAGACTGACCACACCTAGGGAGAAAAAAC |
| | | | TCACTTGAGAGAAAGCTGAGTCCATTGGAAGGGCTTCC |
| | | | AGGAGGATGCCTGGTCTAGGGCCTGCATGGTCAACACA |
| 61 | PHGDH | 2354657 | ATTACTTTTCCCATGGCGAGACCTGCTTTCCCTCCTGCT |
| | | | GGAGGAGGATCTGGGGGAATTTACCTCTGCTCTAACTC |
| | | | CTCCCTGCAGTTTCCATCTGAGCTCTCTGGTATTCACTG |
| | | | ATATTC |
| 62 | PHGDH | 2354660 | GCAGATTCCCCAGGCGACGGCTTCGATGAAGGACGGCA |
| | | | AATGGGA |
| 63 | PHGDH | 2354662 | GAGAGGTAGCTACCCGGATGCAGTCCTTTGGGA |
| 64 | PHGDH | 2354666 | TTCCTTCTCCGTGGCACCACTACACATCAATATTCCTGG |
| | | | CAATATTCTTCATCATGGAGACTTCGGCAGCGACTTCAA |
| | | | CCAGATGAAA |
| 65 | PHGDH | 2354667 | GAAACATGGCTTGGATCATTCCGTCTCCCACCTCAGCCC |
| | | | CTCCGGAGCTGCCTGGACCTCATCATTCCGGAGAGTCT |
| | | | AAGTGGC |
| 66 | PHGDH | 2354674 | CAGTCCCAGCATCATTGTGTGGTCATGAGAATTAATTA |
| | | | AGCTGATCATGGTACTTAGTATATGGTAAATAGTACTTA |
| | | | GTATGTGGAACATGGTACTTAGTGTATGGTAAATTAAC |
| | | | TGGAGAATTA |
| 67 | PHGDH | 2354675 | GGTATGACCCCATCATTTCCCCAGAGGTCTCGGCCTCCT |
| | | | TTGGTGTTCAGCA |
| 68 | PHGDH | 2354677 | CCTTTGCCCAGTGCAAGAAGGGGGTGCGTGTGGTGAAC |
| | | | TGTGCCCGTGGAGGGATCGTGGACGAAGGCGCCCTGCT |
| | | | CC |
| 69 | PHGDH | 2354682 | GCCTTGGTGGACCATGAGAATGTCATCAGCTGTCCCCA |
| | | | CCTGGGTGCCAGCACCAAGGAGGCTCAGAGCCGCTGTG |
| | | | GG |
| 70 | PHGDH | 2354687 | ATGGTTGACTTTACAAGTTATCTCAATAAAAGTGGCCA |
| | | | GATGCCTAACTCAGAA |
| 71 | PHGDH | 2354688 | TGGACTTCGCATGCGTTGATATTTGAAGCACGATCATCA |
| | | | AAACTTTGTGATAATTGATCGTAGTGTTTAGTAACAATG |
| | | | TAAACACTTAAAAAAATTCAAGATAGAAAATAAAAATG |
| | | | AAGGCAAGTTGGGACTGCCAGAGAAGACCCGTCACTCC |
| | | | TCATCCAAGTTATCTGCGACTCCCATATGTTTTGTGTCA |
| | | | AAGACTCACCTTTATTGTGCTGTCCAATCCCTTCCCCAG |
| | | | TGCAGAAACAAGTCTCCCATGGAGGGGGCTGGGGCAG |
| | | | ACACAGTTTGCTGAAAGGAGCAATTTTGAGTGGTTGTG |
| | | | GCATTCTGTGTCCATTTCTGGCTCCACAGCTTTCTTCATT |
| | | | TGTAGGAACAAGTCCTTGTCCTGTTGTTAGTGGCTGATG |
| | | | GAAGTTGTCACCCACCAGGCACCAAGGCAGGAGTGACC |
| | | | CTATACTGTCTTTC |
| 72 | PHGDH | 2354689 | CCAAGCCTTGGATTGGTCTGGCAGAAGCTCTGGGGACA |
| | | | CTGATGCGAGCCTGGGCTGGGT |
| 73 | PHGDH | 2354690 | AATGCTGGGAACTGCCTAAGCCCCGCAGTCATTGTCGG |
| | | | CCTCCTGAAAGAGGCTTCCAAGCAGGCGGATGTGAACT |
| | | | TGGTGAACGCTAAGCTGCTG |
| 74 | PHGDH | 2354695 | TGGGCTTGGTCCAAGGCACTACGCCTGTACTGCAGGGG |
| | | | CTCAATGGA |
| 75 | PHGDH | 2354698 | CAGGCGTGCGGCTGCTGTCCTACCAGACTTCACTGGTGT |
| | | | CAGATGGGGAGACCTGGCACGTCATGGGCATCTCCT |
| 76 | PHGDH | 2354699 | CCACTGTGATCAATAGGGAGAGAAAATCCACATTCTTG |
| | | | GGCTGAACGCGGGCCTCTGACACTGCTTACACT |
| 77 | NUF2 | 2364439 | TCCAGTAGGAGGCGGCAAGTTTGAAAAGTGATGACGGT |
| | | | TGACGTTTGCTGATTTTTGACTTTGCTTGTAGCTGCTCCC |
| | | | CGAACTCGCCGTCTTCCTGTCGGCGGCCGGCACTGT |
| 78 | NUF2 | 2364440 | TGAGCGCGAGAGGACGGAGGAAGGAAGCCTGCAGACA |
| | | | GACGCCTTCTCCATCCCAAGGCGCGGGCAGGTGCCGGG |
| | | | ACGCTGGGCCTGGCGGTGTTTTCGTCGTGCTCAGCGGTG |
| | | | GGAG |
| 79 | NUF2 | 2364441 | TCAAACTATGTAGTTGGAAAGTGTCTTCATCTCTCGTTA |
| | | | ATGAATAAATTGTAACTGAAATTGTACTTCGAAAGAAT |
| | | | GATAGAATTTGGATATTGGAGGAGGTTCCAAAAGGAAA |
| | | | TACTGGAAGTTTGGGAAAGTTAGGAGACTAACTTGGAG |
| | | | CAGAAATTTCATTCAATTATTAAAGGGTTTAGAAGCCT |
| | | | AGCAGAAAAATTTG |
| 80 | NUF2 | 2364442 | AATGTAGCTGAGATTGTGATTCATATTCGCAATAAGAT |
| | | | CTTAACAGGAGCTGATGGTAAAAACCTCACCAAGAATG |
| | | | ATCTTTATCCAAATC |
| 81 | NUF2 | 2364444 | CATGATCTACATGAGAGCCTTACAAATAGTATATGGAA |
| | | | TTCGACTGGAACATTTT |
| 82 | NUF2 | 2364445 | TGAACTCTGAAGTCATGTATCCACATTTAATGGAAGGC |
| | | | TTCTTACCATTCAGCAATTTAGTTACTCATCT |
| 83 | NUF2 | 2364449 | TTTAAGTGGCATTATCAACTTTATTCACTTC |
| 84 | NUF2 | 2364451 | TCCTCTGCGGACAAAATGCAACAGTTAAACGCCGCACA |
| | | | CCAGGAGGCATTAATGAAACTGG |
| 85 | NUF2 | 2364452 | TGAGGACAGGTATTTCATTTTAGCCTT |
| 86 | NUF2 | 2364453 | ATTCAGGAGCTACAACAATCACTAAA |
| 87 | NUF2 | 2364457 | AAAATGAAAGATACGGTCCAGAAGCTTAAA |
| 88 | NUF2 | 2364459 | ATCTATGGAGACTCAGTTGACTGCCTGCCTTCATGTCAG |
| | | | TTGGAAGTGCAGTTATATCAAAAGAAAATACAGGACCT |
| | | | TTCAGATAATAGGGAAAAATTAGCCAGTATCTTAAAGG |
| | | | AG |
| 89 | NUF2 | 2364460 | CGTTCAAAAGACTGATGATTGTGAAGAAGGAAAAACTT |
| | | | GCCACAGCACAATTCAAAATAAATAAGAAGCATGAAG |
| | | | ATGTTAAGCAATACAAACGCACAGTAATTG |
| 90 | NUF2 | 2364461 | AAAAAGAGGTGCTGTCTATGAACGAGTAACCACAATTA |
| | | | ATCAAGA |
| 91 | NUF2 | 2364463 | AAAACTGCTTTGGAGAAATACCACGACGGTATTGAAAA |
| | | | GGCAGCAGAGGACTCCTATGCTAAGATAGATGAGAAG |
| | | | ACAGCTGAACTG |
| 92 | NUF2 | 2364464 | AAAAGTTGAAGCGAATGGAAGTATCAGAAGTACCAAA |
| | | | TAATGTTGGCTTCATCAGTTTTTATACACTCTCATAAGT |
| | | | AGTTAATAAGATGAATTTAATGTAGGCTTTTATTAATTT |
| | | | ATAATTAAAATAACTTGTGCAGCTATTCATGTC |
| 93 | CENPF | 2379864 | GCTGCGGGCAGTTTGAATTAGACTCTGGGCTC |
| 94 | CENPF | 2379865 | CGCGCCAGAACTGTACTCTCCGAGAGGTCGTTTTCC |
| 95 | CENPF | 2379867 | TGTCCCAGACCCTACTCGGTCACGGACTCACACTTTAGG |
| | | | GGATCATTTTCTTCCTCCGTAAAAGAATTGGAGATGACT |
| | | | A |
| 96 | CENPF | 2379878 | TTTACAAATGTTGGAGTAATAAAGAAGGCAGAAC |
| 97 | CENPF | 2379879 | GAAAGAAGGGCTGCCTACAAGAGCTCTTCAGAAAATTC |
| | | | AAGAGCTTGAAGGACAGCTTGACAAACTGAAGAAGGA |
| | | | AAAGCAGCAAAGGCAGTTTCAGCTTGACAGTCTCGAGG |
| | | | CTGCGCTGCAGAAGCAAAAA |
| 98 | CENPF | 2379880 | GAAGATTTCTCATGAACTTCAAGTCAAGGAGTCACAAG |
| | | | TGAATTTCCAGGAAGGACAACTGAATTCAGGCAAAAAA |
| | | | CA |
| 99 | CENPF | 2379881 | TATTTTGGGATGGTATTTATAGGGATGATATTTGTATGT |
| | | | ATTAATCAGATGCGTTTGTCTTTTCCTTTAACAATATAA |
| | | | TTATTATACTTTGCAATTTTTTTTCCTGGTAGAATAAGT |
| | | | AATGATTCGGTCTCTGTACC |
| 100 | CENPF | 2379882 | GAAGCCAACAAGCTGCGCAGTCTGCAGATG |
| 101 | CENPF | 2379885 | AGACTCTTCCACAAGCCACCATGAATCACCGCGACATT |
| | | | GCCCGGCATCAGGCTTCATCATCTGT |
| 102 | CENPF | 2379886 | TAGGAGAGATTTCTCTGCATCTTACTTTTCTGGGGAACA |
| | | | AGAGGTGACTCCAAGTCGATCAACTTTGCAAATAGGGA |
| | | | AAAGAGATGCTAATAGCAGTTTCTTTGACAATTCTAGC |
| | | | AGTCCTCATCTTTTGGATCAATTAAAAGCGCAGAATC |
| 103 | CENPF | 2379888 | ACTGAAAAAATTGACGGAAGATTTGAGTTGTCAGCGAC |
| | | | AAAATGCAGAA |
| 104 | CENPF | 2379889 | GAGCTCTCCCGTCAACAGCGTTCTTTCCAAACACTGGAC |
| | | | CAGGAGTGCATCCAGATGAAGGCCAGACTCACCCAGGA |
| | | | GTTACAGCAAGCCAAGAATATGCACAACGTCCTGCAGG |
| | | | CTG |
| 105 | CENPF | 2379890 | AGAGTTTAAGCAAAAGTTGTGCAGAGCTGAACAGGCGT |
| | | | TCCAGGCGAGTCAGATCAAGGA |
| 106 | CENPF | 2379895 | AAAGGCCAGAGAAGTCTGCCACCTGGAGGCAGAACTC |
| | | | AAGAACATCAAACAGTGTTTAAATCAGAGCCAGAATTT |
| | | | TGCAGAAGA |
| 107 | CENPF | 2379896 | GCCTTGCTGAGTGCTTTAGAGTTAAAAAAGAAAGAATA |
| | | | TGAAGAATTGAAAGAAGAGAAAACTCTGTTTTCTTGTT |
| | | | GGAAAAGTGAAAACGAAAAACTTTTAACTCAGATGGA |
| | | | ATCAGAAAAGGAAAACTTGCAGAGTAAAATTAATCACT |
| | | | TGGAAACTTGTCTGAAGACACAGCAAATAAAAAGTCAT |
| | | | GAATACAACGAGAGAGTAAGAACGCTGGAGATGGACA |
| | | | GAGAAAACCTAAGTGTCGAGATCAGAAACCTTCACAAC |
| | | | GTGTTAGACAGTAAGTCAGTGGAGGTAGAGACCCAGAA |
| | | | ACTAGCTTATATGGAGCTACAGCAGAAAGCTGAGTTCT |
| | | | CAGATCAGAAACATCAGAAGGAAATAGAAAATATGTG |
| | | | TTTGAAGACTTCTCAGCTTACTGGGCAAGTTGAAGATCT |
| | | | AGAACACAAGCTTCAGTTACTGTCAAATGAAATAATGG |
| | | | ACAAAGACCGGTGTTACCAAGACTTGCATGCCGAATAT |
| | | | GAGAGCCTC |
| 108 | CENPF | 2379897 | TTTGGCTTTTGATCAGCAGCCTGCCATGCATCATTCCTT |
| | | | TGCAAATATAATTGGAGAACAAGGAAGCATGCCTTCAG |
| | | | AGAGGAGTGAATGTCGTTTAGAAGCAGACCAAAGTCCG |
| | | | AAAAATTCTGCCATCCTACAAAATAGAGTTGATTCACTT |
| | | | GAATTTTCATTAGAGTCTCAAAAACAGATGAACTCAGA |
| | | | CCTGCAAAAGCAGTGTGAAGAGTTGGTGCAAATCAAAG |
| | | | GAGAAATAGAAGAAAATCTCATGAAAGCAGAACAGAT |
| | | | GCATCAAAGTTTTGTGGCTGAAACAAGTCAGCGCATTA |
| | | | GTAAGTTACAGGAAGACACTTCTGCTCACCAGAATGTT |
| | | | GTTGCTGAAACCTTAAGTGCCCTTGAGAACAAGGAAAA |
| | | | AGAGCTGCAACTTTTAAATGATAAGGTAGAAACTGAGC |
| | | | AGGCAGAGATTCAAGAATTAAAAAAGAGCAACCATCT |
| | | | ACTTGAAGACTCTCTAAAGGAGCTACAACTTTTATCCG |
| | | | AAACCCTAAGCTTGGAGAAGAAAGAAATGAGTTCCATC |
| | | | ATTTCTCTAAATAAAAGGGAAATTGAAGAGCTGACCCA |
| | | | AGAGAATGGGACTCTTAAGGAAATTAATGCATCCTTAA |
| | | | ATCAAGAGAAGATGAACTTAATCCAGAAAAGTGAGAG |
| | | | TTTTGCAAACTATATAGATGAAAGGGAGAAAAGCATTT |
| | | | CAGAGTTATCTGATCAGTACAAGCAAGAAAAACTTATT |
| | | | TTACTACAAAGATGTGAAGAAACCGGAAATGCATATGA |
| | | | GGATCTTA |
| 109 | CENPF | 2379898 | ACTGTGAAATAGATGCGGAAGAAAAGTATATTTCAGGG |
| | | | CCTCATGAGTTGTCAACAAGTCAAAACGACAATGCACA |
| | | | CCTTCAG |
| 110 | CENPF | 2379899 | TCTGCAAACAACAATGAACAAGCTGAATGAGCTAGAGA |
| | | | AAATATGTGAAATACTGCAGGCTGAAAAGTATGAACTC |
| | | | GTAACTGAGCTGAATGATTCAAGGTCAGAATGTATCAC |
| | | | AGCAACTAGGAAAATGGCAGA |
| 111 | CENPF | 2379900 | TGGCTCCATTGGACGAGAGTAATTCCTACGAGCACTTG |
| | | | ACATTGTCAGACAAAGAAGTTCAAATGCACTTTGCCGA |
| | | | ATTGCAAGAGAAATTCTTATCTTTACAAAGTGAACACA |
| | | | AAATTTTACATGATCAGCACTGTCAGATGAGCTCTAAA |
| | | | ATGTCAGAGCTGCAGACCTATGTTGACTCATTAAAGGC |
| | | | CGAAAATTTGGTCTTGTCAACGAATCTGAGAAACTTTC |
| | | | AAGGTGACTTGGTGAAGGAGATGCAGCTGGGCTTGGAG |
| | | | GAGGGGCTCGTTCCATCCCTGTCATCCTCTTGTGTGCCT |
| | | | GACAGCTCTAGTCTTA |
| 112 | CENPF | 2379901 | GCTGACAAGCGTGACTCTGGAGATGGAGTCCAAGTTGG |
| | | | CGGCAGAAAAGAAACAGACGGAACAACTGTCACTTGA |
| | | | GCTGGAAGTAGCACGACTCCAGCTACAAGGTCTGGACT |
| | | | TAAGTTCTCGGTCTTTGCTTGGCATCGACA |
| 113 | CENPF | 2379902 | AGATACCAATTATGAGCCTCCAGGGGAAGATAAAACCC |
| | | | AGGGCTCTTCAGAATGCATTTCTGAATTGTCATTTTCTG |
| | | | GTCCTAATGCTTTGGTACCTATGGATTTCCTGGGGAATC |
| | | | AGGAAGATATCCATAATCTTCAACTGCGGGTAAAAGAG |
| | | | ACATCAAATGAGAATTTGAGATTACTTCATGTGATAGA |
| | | | GGACCGTGACAGAAAAGTTGAAAGTTTGCTAAATGAAA |
| | | | TGAAAGAATTAGACTCAAAACTCCATTTACAGGAGGTA |
| | | | CAACTAATGACCAAAATTGAAGCATGCATAGAATTGGA |
| | | | AAAAATAGTTGGGGAACTTAAGAAAGAAAACTCAGATT |
| | | | TAAGTGAAAAATTGGAATATTTTTCTTGTGATCACCAGG |
| | | | AGTTACTCC |
| 114 | CENPF | 2379903 | ATTGAGCATGAAGCCCTCTACCTGGAGGCTGACTTAGA |
| | | | GGTAGTTCAAACAGAGAAGCTATGTTTAGAAAAAGACA |
| | | | ATGAAAATAAGCAGAAGGTTATTGTCTGCCTTGAAGAA |
| | | | GAACTCTCAGTGGTCACAAGTGAGAGAAACCAGCTTCG |
| | | | TGGAGAATTAGATACTATGTCAAAAAAAACCACGGCAC |
| | | | TGGATCAGTTGTCTGAAAAAATGAAGGAGAAAACACA |
| | | | AGAGCTTGAGTCTCATCAAAGTGAGTGTCTCCATTGCAT |
| | | | TCAGGTGGCAGAGGCAGAGGTGAAGGAAAAGACGGAA |
| | | | CTCCTTCAGACTTTGTCCTCTGATGTGAGTGAGCTGTTA |
| | | | AAAGACAAAACTCATCTCCAGGAAAAGCTGCAGAGTTT |
| | | | GGAAAAGGACTCACAGGCACTGTCTTTGACAAAATGTG |
| | | | AGCTGGAAAACCAAATTGCACAACTGAATAAAGAGAA |
| | | | AGAATTGCTTGTCAAGGAATCTGAAAGCCTGCAGGCCA |
| | | | GACTGAGTGAATCAGATTATGAAAAGCTGAATGTCTCC |
| | | | AAGGCCTTGGAGGCCGCACTGGTGGAGAAAGGTGAGTT |
| | | | CGCATTGAGGCTGAGCTCAACACAGGAGGAAGTGCATC |
| | | | AGCTGAGAAGAGGCATCGAGAAACTGAGAGTTCGCATT |
| | | | GAGGCCGATGAAAAGAAGCAGCTGCACATCGCAGAGA |
| | | | AACTGAAAGAACGCGAGCGGGAGAATGATTCACTTAA |
| | | | GGATAAAGTTGAGAACCTTGAAAGGGAATTGCAGATGT |
| | | | CAGAAGAAAACCAGGAGCTAGTGATTCTTGATGCC |
| 115 | CENPF | 2379904 | AGAGTCTAGACCCACCAATAGAGGAAGAGCATCAGCTG |
| | | | AGAAATAGCATTGAAAAGCTGAGAGCCCGCCTAGAAG |
| | | | CTGATGAAAAGAAGCAGCTCTGTGTCTTACAACAACTG |
| | | | AAGGAAAGTGAGCATCATGCAGATTTACTTAAGGGTAG |
| | | | AGTGGAGAACCTTGAAAGAGAGCTAGAGATAGCCAGG |
| | | | ACAAACCAAGAGCATGCAGCTCTTGAGGCAGAGAATTC |
| | | | CAAAGGAGAGGTAGAGACCCTAAAAGCAAAAATAGAA |
| | | | GGGATGACCCAAAGTCTGAGAGGTCTGGAATTA |
| 116 | CENPF | 2379905 | GCTCAATGAGAGAGTGGCAGCCCTGCATAATGACCAAG |
| | | | AAGCCTGTAAGGCCAAAGAGCAGAATCTTAGTAGTCAA |
| | | | GTAGAGTGTCTTGAACTTGAGAAGGCTCAGTTGCTACA |
| | | | AGGCCTTGATGAGGCCAAAAATAATTATATTGTTTTGC |
| | | | AATCTTCAGTGAATGGCCTCATTCAAGAAGTAGAAGAT |
| | | | GGCAAGCAGAAACTGGAGAAGAAGGATGAAGAAATCA |
| | | | GTAGACTGAAAAATCAAATTCAAGACCAAGAGCAGCTT |
| | | | GTCTCTAAACTGTCCCAGGTG |
| 117 | CENPF | 2379907 | AGAGAAAAATAGGCTAGCTGGAGAGTTGC |
| 118 | CENPF | 2379908 | ATAGTGAATTGAAGAAGAGCCTAGATTGCATGCACAAA |
| | | | GACCAGGTGGAAAAGGAAGGGAAAGTGAGAGAGGAAA |
| | | | TAGCTGAATATCAGCTACGGCTTCATGAAGCTG |
| 119 | CENPF | 2379909 | AATCCAGACATACCGAGAGAAATTGACTTCTAAAGAAG |
| | | | AATGTCTCAGTTCACAGAAGCTGGAGATAGACCTTTTA |
| | | | AAGTCTAGTAAAGAAGAGCTCAATAATTCATTGAAAGC |
| | | | TACTACTCA |
| 120 | CENPF | 2379910 | TCTTCCTTTGGAAATTCATGATGCCATATCAGATGGTTT |
| | | | TAGAATTCTGCACTTTAATAATGTAATGCATATGCCATA |
| | | | TATAATATCCCAGAGGGATCTGCTATAATATTGCATAAT |
| | | | CGAATTCATATTTCTGCAGCAAAATGTGTGGATACTCTC |
| | | | ACAAGGCAGGATAAATAGA |
| 121 | CENPF | 2379911 | TTTTTCCATATGCTTATAAAAAGAAATTCA |
| 122 | CENPF | 2379912 | ATGGACAATCTAAAATATGTAAATC |
| 123 | CENPF | 2379913 | GAAGTTGTTGATCAAATCCTGTAAACAGCTGGAAGAGG |
| | | | AAAAGGAGATACTGCAGAAAGAACTCTCTCAACTTCAA |
| | | | GCTGCACAGGAGAAGCA |
| 124 | CENPF | 2379914 | TCTAAACAAGATTCCCGAGGGTCTCCTTTGCTAGGTCCA |
| | | | GTTGTTCCAGGACCATCTCCAATCCCTTCTGTTACTGAA |
| | | | AAGAGGTTATCATCTGGCCAAAATAAAGCTTCAGGCAA |
| | | | GAGGCAAAGATCCAGTGGAATATGGGAGAATGGTAGA |
| | | | GGACCAACACCTGCTACCCCAGAGAGCTTTTCTAAAAA |
| | | | AAGCAAGAAAGCAGTCATGAGTGGTATTCACCCTG |
| 125 | CENPF | 2379915 | GACTAGCCCATATATCCTGCGAAGAACAACCATGGCAA |
| | | | CTCGGACCAGCCCCCGCCTGGCTGCACAGAAGTTAGCG |
| | | | CTATCCCCACTGAGTCTCGGCAAAGAAAATCTTGCAGA |
| | | | GTCCTCCAAACCAACAGCTGGTG |
| 126 | CENPF | 2379918 | CTCAGCGGAGCCCAGTAGATTCAGGCACCATCCTCCGA |
| | | | GAACCCACCACGAAATCCGTCCCAGTCAATAATCTTCC |
| | | | TGAGAGAAGTCCGACTGACAGCCCCAGAGAGGGCCTG |
| | | | AGGGTCAAGCGAGGCCGACTTGTCCCCAGCCCCAAAGC |
| | | | TGGACTGGAGTCCAACGGCAGTGAGAAC |
| 127 | CENPF | 2379919 | TTCTCTTTAGTCAGGGCATGCTTTATTAGTGAGGAGAAA |
| | | | ACAATTCCTTAGAAGTCTTAAATATATTGTACTCTTTAG |
| | | | ATCTCCCATGTGTAGGTATTGAAAAAGTTTGGAAGCAC |
| | | | TGATCACCTGTTAGCATTGCCATTCCTCTACTG |
| 128 | CENPF | 2379920 | CTTCCTAGAGGTGTGCTATACCATGCGTCTGTCGTTGTG |
| | | | CTTTTTTCTGTTTTTAGACCAATTTTTTACAGTTCTTTGG |
| | | | TAAGCATTGTCGTATCTGGTGATGGATTAACATATAGCC |
| 129 | EXO1 | 2388223 | GGCCATCAGCGCCAGTGCCACTCGCGCCCTCAAG |
| 130 | EXO1 | 2388225 | TCGGAGCGGGTTTCTCCAACCGCAATCGGCTCCGCTCA |
| | | | AGGGGAGGA |
| 131 | EXO1 | 2388226 | AAACGTGTCGTCTGGAATGGGCTTGGGGGCCACGCCTG |
| | | | CACATCTCCGCGAGACAGAGGGATAAAGTGAAGATGGT |
| | | | GCTGTTATTGTTACCTCGAGTGCCACATGCGACCTCTGA |
| | | | GATATGTACACAGTCATTCTTACTATCGCACTCAGCCAT |
| | | | TCTTACTACGCTAAAGAAGAAATAATTATTCGAGGATA |
| | | | TTTGCCTGGCCC |
| 132 | EXO1 | 2388227 | TAGTGAATCCCAGTCACTGAGTGGAGTTGAGAGTCTAA |
| | | | GAACCTCTGAAATTTGAGAACTGCTGGACCAGAGCCTT |
| | | | TAGAGCTCTGATAAGGTGTC |
| 133 | EXO1 | 2388229 | TGGGGATACAGGGATTGCTACAATTTATCAAAGAAGCT |
| | | | TCAGAACCCATCCATGTGAGGAAGTATAAAGGGCAGGT |
| | | | AGTAGCTGTGGATACATATTGCTGGCTTCACAAAGGAG |
| | | | CTATTGCTTGTGCTGAAAAACTAGCCAAAGGTGAACCT |
| | | | ACTGATA |
| 134 | EXO1 | 2388230 | CTATCTCATGGGATCAAGCCTATTCTCGTATTTGATGGA |
| | | | TGTACTTTACCTTCTAAAA |
| 135 | EXO1 | 2388231 | AGACGACAAGCCAATCTTCTTAAGGGAAAGCAACTTCT |
| | | | TCGTGAGGGGAAAGTCTCGGAAGCTCGAGAGTGTTTCA |
| | | | CCCGGTC |
| 136 | EXO1 | 2388232 | CTCGTGGCTCCCTATGAAGCTGATGCGCAGTTGGCCTAT |
| | | | CTTAACAAAGCGGGAATTGTGCAAGCCATAATTACAGA |
| | | | GGACTCGGATCTCCTAGCTTTTGGCTGTA |
| 137 | EXO1 | 2388233 | TGAAATTGATCAAGCTCGGCTAGGAATGTGCAGACAGC |
| | | | TTGGGGATGTATTCACGGAAGAGAAGTTTCGTTACATG |
| | | | TGTATTCTTTCAGGTTGTGACTACCTGTCATCACTGCGT |
| | | | GGGATTGGA |
| 138 | EXO1 | 2388234 | TATCACGGTACCAGAGGATTACATCAACGGGTTTATTC |
| | | | GGGCCAACAATACCTTCCTCTATCAGCTAGTTTTTGATC |
| | | | CCATCAAAAGGAAACTTATTCCTCTGAACGCCTATGAA |
| | | | GATGATGTTGATCCTGAAACACTAAGCTACGCTGGG |
| 139 | EXO1 | 2388235 | TGATGATTCCATAGCTCTTCAAATAGCACTTGGAAATA |
| | | | AAGATATAAATACTTTTGAACAGATCGATGACTACAAT |
| | | | CCAGACACTGC |
| 140 | EXO1 | 2388238 | CATAGTTGGGATGACAAAACATGTCAAAAGTCAGCTAA |
| | | | TGTTAGCAGCATTTGGCATAGGAATTACTCTCCCAGACC |
| | | | AGAGTCGGGTACTGTTTCAGATGCCCCACAATTGAAGG |
| | | | AAAATCCAAGTACTGTGGGAGTGGAACGA |
| 141 | EXO1 | 2388239 | ACCTGTTGAGTCAGTATTCTCTTTCATTTACGAAGAAGA |
| | | | CCAAGAAAAATAGCTCTGAAGGCAATAAATCATTGAGC |
| | | | TTTTCTGAAGTGTTTGTGCCTGACCTGGTAAATGGACCT |
| | | | ACTAACAAAAAGAGTGTAAGCACTCCACCTAGGACGA |
| 142 | EXO1 | 2388240 | GTGGTGCAGTTGTGGTTCCAGGGACC |
| 143 | EXO1 | 2388242 | GCCTCTGGATGAAACTGCTGTCACAGATAAAGAGAACA |
| | | | ATCTGCATGAATCAGAGTATGGAGACCAAGAAGGCAA |
| | | | GAGACTGGTTGACACAGATGTAGCACGTAATTCAAGTG |
| | | | ATGACATTCCGAATAATCATATTCCAGGTGATCATATTC |
| | | | CAGACAAGGCAACAGTGTTTACAGATGAAGAGTCCTAC |
| | | | TCTTTTGAGAGCAGCAAATTTACAAGGACCATTTCACC |
| | | | ACCCACTTTGGGAACACTAAGAAGTTGTTTTAGTTGGTC |
| | | | TGGAGGTCTTGGAGATTTTTCAAGAACGCCGAGCCCCT |
| | | | CTCCAAGCACAGCATTGCAGCAGTTCCGAAGAAAGAGC |
| | | | GATTCCCCCACCTCTTTGCCTGAGAATAATATGTCTGAT |
| | | | GTGTCGCAGTTAAAGAGCGAGGAGTCCAGTGACGATGA |
| | | | GTCTCATCCCTTACGAGAAGAGGCATGTTCTTCACAGTC |
| | | | CCAGGAA |
| 144 | EXO1 | 2388244 | TCTGATTGCAATATTAAGTTACTTGACAGTCAAAGTGAC |
| | | | CAGACCTCCAAGCTACGTTTATCTCATTTC |
| 145 | EXO1 | 2388246 | GGCTATATAAGTCCAGTTCTGCAGACTCTCTTTCTACAA |
| | | | CCAAGATCAAACCTCTAGGACCTGCCAGAGCCAGTGGG |
| | | | CTGAGCAAGAAGCCGGCAAGCATCCAGAAGAGAAAGC |
| | | | ATCATAATGCCGAGAACAAGCCGGGG |
| 146 | EXO1 | 2388248 | AGAGATAACATCCAACTAACTCCAGAAGCGGAAGAGG |
| | | | ATATATTTAACAAACCTGAATGTGGCCGTGTTCAAAGA |
| | | | GCAATATTCCA |
| 147 | EXO1 | 2388253 | TCTCGCTGTGTCACAATCTCAGCTCACT |
| 148 | EXO1 | 2388254 | TTCCTCCATCCTAGGCAGAAATAAAGTCCCAAATCTTTG |
| | | | TTTTTTAACGGGTCATAGAGGACCCATCATCACCCTTTA |
| | | | TTCATTCCTTGATCATCTCAGGCTAGAGAAGTCTAGGGA |
| | | | TACAGCT |
| 149 | UBE2T | 2451201 | TGTCCTGGTTCATCTTAGTTAATGTGTTCTTTGCCAAGG |
| | | | TGATCTAAGTTGCCTACCTTGAATTT |
| 150 | UBE2T | 2451202 | ACCAGAGGCTGGTGACTCCAGAGTACACAACTCAACAC |
| | | | AGAAAAGGAAGGCCAGTCAGCTAGTAGGCATAGAAAA |
| 151 | UBE2T | 2451205 | ATTTAAATATAATAAGCCAGCCTTCCTCAAGAAT |
| 152 | UBE2T | 2451206 | GCTCTAACCACTGCAAATCATGTTTTTCT |
| 153 | UBE2T | 2451207 | CTCTGCAACACATATCCTACCTTGTCTATACCGCTAACT |
| | | | CTC |
| 154 | UBE2T | 2451208 | CATCCCTCAACATCGCAACTGTGTTGACCTCTATTCAGC |
| | | | TGCTCATGTCAGAACCCAACCCTGATGACCCGCTCATG |
| | | | GCTGACATA |
| 155 | UBE2T | 2451210 | TGAACCTCCTCAGATCCGATTTCTCACTCCAATTTATCA |
| | | | TCCAAACATTGATTCTGCTGGAAGGATTTGTCTGGATGT |
| | | | TCTCAAATTGCC |
| 156 | UBE2T | 2451214 | TGGAGCCAACACACCTTATGAGAAAGGTGTTTTTAAGC |
| | | | TAGAAGTTATCATTCCTGAG |
| 157 | UBE2T | 2451215 | AGAGCTTCACGTCTGAAGAGAGAGCTGCACATGTTAGC |
| | | | CACAGAGCCACCCCCAGGCATCACATGTTGGCAAGATA |
| | | | AAGACCAAATGGATGACCTGCGAGCTC |
| 158 | UBE2T | 2451216 | GTGTGTGGTTCCTTCTACTTGGGGATC |
| 159 | RRM2 | 2469253 | TCCCGCGCTGCGCTTGAAAATCGCGCGCGGCCCCGCGG |
| | | | CCAGCCTGGGTAGGGGCAAGGCGCAGCCAATGGGAAG |
| | | | GGTCGGAGGCATGGCACAGCCAATGGGAAGGGCCGGG |
| | | | GCACCAAAGCCAATGGGAAGGGCCGGGAGCGCGCGGC |
| | | | GCGGGAGATTTAAAGGCTGCTGGAGTGAGGGGTCGCCC |
| | | | GTGCACCCTGTCCCAGCCGTCCTGTCCTGGCTGCTCGCT |
| | | | CTGCTTCGCTGCGCCTCCACT |
| 160 | RRM2 | 2469254 | AGCTGCAGCTCTCGCCGCTGAAGGGGC |
| 161 | RRM2 | 2469255 | GACCCGCGTCCTGGCCAGCAAGACCGCGAGGAGGATCT |
| | | | TCCAGG |
| 162 | RRM2 | 2469256 | TCTGCTGCGACCCACGGAGTGCGACGGGACAGCCACGT |
| | | | TTTCACATCGGGCCCCGTGAAATTGCCGCCAATGGAAA |
| | | | GGACTTGGTCCAGAAAAACGTTAGTTTCATATGGTTCG |
| | | | CCCGGTACTTA |
| 163 | RRM2 | 2469257 | GCTGCCCCCGGCGTGGAGGATGAGCCGCTGCTG |
| 164 | RRM2 | 2469258 | CCCGCCGCTTTGTCATCTTCCCCATCGAGTACCATGATA |
| | | | TCTGGCAGATGTATAAGAAGGCAGAGGCTTCCTTTTGG |
| | | | ACCGCCGAG |
| 165 | RRM2 | 2469259 | ATCGGAGGACCCCAGAAGACCCCTGCAGGG |
| 166 | RRM2 | 2469260 | CCCGAGGAGAGATATTTTATATCCCA |
| 167 | RRM2 | 2469261 | TGACGATCTGAGGTCGAACTAGTTCGCTTTCCTCGTCTT |
| | | | GTATGTTTTTCCATGCTGAGTGCATCTGTGTGTGTAAGC |
| | | | TGGGTTTTATATTACATGGCATTTCCTGTTTTGTAACAC |
| | | | TTTGCAGTTCTTTCTTATGGTATTTTCCCGACTCTAGAG |
| | | | AAGCTGAGACAATATTAAGTGGTAGCAATGTGATGACT |
| | | | CTTTGTGGCC |
| 168 | RRM2 | 2469263 | AGTCTTCTTATTGACACTTACATAAAAGATCCCAAAGA |
| | | | AAG |
| 169 | RRM2 | 2469266 | CCCTGCTGTACTGGACTATGTTTTACTGTCTGTAGACCC |
| | | | TGAAGCTCAATATGAACTACAGAATACCCAAACTTGTA |
| | | | TTAATGTAAATCAAGTGTTGAGGTTTTTAAAAGAACAC |
| | | | TGGAGGGAAAAACTGACCAGTAAAAATAAAACATTTCG |
| | | | GTGTGAGTTCTTCCTTTAGGAAGAGGATTGGCAAATAC |
| | | | TTGAATTTGGCCTTTGTCCCAGAGCTCTTATCTAGCAGT |
| | | | TGGTAATCGGAGGTCTTTTACTGTAATGCTTCAATTGCT |
| | | | GATACCGTATGTG |
| 170 | RRM2 | 2469267 | AACGATGCCTTGTGTCAAGAAGAAGGCAGACTGGGCCT |
| | | | TGCGCTGGATTGGGGACAAAG |
| 171 | RRM2 | 2469269 | TCGATATTCTGGCTCAAGAAACGAGGAC |
| 172 | RRM2 | 2469270 | CAACTCGGGCATGCTCTTGTGTTCACTGACGGGGACCT |
| | | | GAGATGCTAGATGGCATATATCCACATTTA |
| 173 | RRM2 | 2469273 | ACACCTGGTACACAAACCATCGGAGGAG |
| 174 | RRM2 | 2469275 | GCTCATTGGGATGAATTGCACTCTAATGAAGCAATACA |
| | | | TTGAGTTTGTGGCAGACAGACTTATGCTGGAACTGGGT |
| | | | TTTAG |
| 175 | RRM2 | 2469276 | AGTAGAGAACCCATTTGACTTTATGGAGAATATTTCACT |
| | | | GGAAGGAAAGACTAACTTCTTTGAGAAGAGAGTAGGC |
| | | | GAGTATCAGAGGATGGGAGTGATGTCAAGTCCAACAGA |
| | | | GAATTCTTTTACCTTGGATGC |
| 176 | RRM2 | 2469277 | TGAAGATGTGCCCTTACTTGGCTGATTTTTTTTTTCCATC |
| | | | TCATAAGAAAAATCAGCTGAAGTGTTACCAACTAGCCA |
| | | | CACCATGAATTGTCCGTAATGTTCATTAACAGCATCTTT |
| | | | AAAACTGTGTAGCTACCTCACAACCAGTCCTGTCTGTTT |
| | | | ATAGTGCTGGTA |
| 177 | RRM2 | 2469278 | CTTTAGTGAGCTTAGCACAGCGGGATTAAACAGTCCTTT |
| | | | AACCAGCACAGCCAGTTAAAAGATGCAGCCTCACTGCT |
| | | | TCAACGCAGATT |
| 178 | RRM2 | 2469279 | AGTCAGTCCTGTGTATACCTAGATATTAGTCAGTTGGTG |
| | | | CCAGATAGAAGACAGGTTGTGTTTTTATCCTGTGGCTTG |
| | | | TGTAGTGTCCTGGGATTCTCTGCCCCCTCTGAGTAGAGT |
| | | | GTTGTGGGATAAAGGAATCTCTCAGGGCAAGGAGCTTC |
| | | | TTAAGTTAAATCACTAGAAATTTAGGGGTGATCTGGGC |
| | | | CTTCATATGTGTGAGAAGCCGTTTCATTTTATTTCTCAC |
| | | | TGTATTTTCCTCAACGTCTGGTTGATGAGAAAAAATTCT |
| | | | TGAAGAGTTTTCATATGTGGGAGCTAAGGTAGTATTGT |
| | | | AAAATTTCAAGTCATCCTTAAACAAAATGATCCACCTA |
| | | | AGATCTTGCCCCTGTTAAGTGGTGAAATCAACTAGAGG |
| | | | TGGTTCCTACAAGTTGTTCATTCTAGTTTTGTTTGGTGTA |
| | | | AGTAGGTTGTGTGAGTTAATTCATTTATATTTACTATGT |
| | | | CTGTTAAATCAGAAATTTTTTATTATCTATGTTCTTCTAG |
| | | | ATTTTACCTGTAGTTCATACTTCAGTCACCCAGTGTCTT |
| | | | ATTCTGGCATTGTCTAAATCTGAGCATTGTCTAGGGGGA |
| | | | TCTTAAACTTTAGTAGGAAACCATGAGCTGTTAATACA |
| | | | GTTTCCATTCAAATATTAATTTCAGAATGAAACATAATT |
| | | | TTTTTTTTTTTTTTTGAGATGGAGTCTCGCTCTGTTGCCC |
| | | | AGGCTGGAGTGCAGTGGCGCGATTTTGGCTCACTGTAA |
| | | | CCTCCATCTCCTGGGTTCAAGCAATTCTCCTGTCTCAGC |
| | | | CTCCCTAGTAGCTGGGACTGCAGGTATGTGCTACCACA |
| | | | CCTGGCTAATTTTTGTATTTTTAGTAGAGATGGAGTTTC |
| | | | ACCATATTGGTCAGGCTGGTCTTGAACTCCTGACCTCAG |
| | | | GTGATCCACCCACCTCGGCCTCCCAAAGTGCTGGGATT |
| | | | GCAGGCGTGATAAACAAATATTCTTAATAGGGCTACTT |
| | | | TGAATTAATCTGCCTTTATGTTTGGGAGAAGAAAGCTG |
| | | | AGACATTGCATGAAAGATGATGAGAGATAAATGTTGAT |
| | | | CTTTTGGCCCCATTTGTTAATTGTATTCAGTATTTGAAC |
| | | | GTCGTCCTGTTTATTGTTAGTTTTCTTCATCATTTATTGT |
| | | | ATAGACAATTTTTAAATCTCTGTAATATGATACATTTTC |
| | | | CTATCTTTTAAGTTATTGTTACCTAAAGTTAATCCAGAT |
| | | | TATATGGTCCTTATATGTGTACAACATTAAAATGAAAG |
| | | | GCTTTGTCTTGCATTGTGAGGTACAGGCGGAAGTTGGA |
| | | | ATCAGGTTTTAGGATTCTGTCTCTCATTAGCTGA |
| 179 | RRM2 | 2469280 | GTCTCAAAATTGAATAATGCACAAGTCTTAAGTGATTA |
| | | | AAATA |
| 180 | MLPH | 2534256 | TGAGCACCCAAAGGCCGGCCCTAGAGTCCAGGAGAAG |
| | | | AGCGCAGCGGCGCGGAGCTCCCAGGCGTTCCCCGCAGC |
| | | | GCGTCCTCGGTCCTGGAACCACCGCGCCGCGCGTCCTG |
| | | | GCTTCCACATCTGCCCCATTTGCCCGCGGATCTTGACTT |
| | | | TTTCTTGGCGGGCAAGGCC |
| 181 | MLPH | 2534257 | GAGAGCCAGGCGCTAACCAGCCGCTCTGCGCCCCGCGC |
| | | | CCTGCTTGCCCCCATTATCCAGCCTTGCCCCGGCGCCCT |
| | | | GACCTGACGCCCTGGCCTGACGCCCTGCTTCGTCGCCTC |
| | | | CTT |
| 182 | MLPH | 2534258 | TGCTGGACCAGGGACTGAGCGTCCCCCGGAGAGG |
| 183 | MLPH | 2534261 | TTGGAAGTTGTTCAACGAGATTTTGACCTCCGAAGG |
| 184 | MLPH | 2534266 | CAAACCTGGGGCGTTAGCTCAACTCTTGCCCCCCTGCTG |
| | | | AAGGAGACCAAAACAATGCTTGATCAGGAAACACCATC |
| | | | TGGCTTTGCCCCCAGGATTCTGTGACTGCCCTGGGGAG |
| | | | GGCGCAGTGACCTGCCAACCAAAATTGGTACAATTGTA |
| | | | AACAGCCACAGAAATGCTTAAATGCAATATCATTTCTA |
| | | | TGAAATTAACGTGTTTCCATTCCATTCCAGCCACCAAAA |
| | | | TTGCCCGTTTGAGCTCAGCCCTCAAAACAAAGATGCCT |
| | | | GTGTGGCTTTGCCCAACGTTGGGTCACTGTTTTCTGCAT |
| | | | A |
| 185 | MLPH | 2534267 | ATGCCCAATATATTTCTTGTTTCTGATATT |
| 186 | MLPH | 2534268 | CGTTGAAGGGCAAGATTAAGAAGGAAAGCTCC |
| 187 | MLPH | 2534269 | AAGAGGGAGCTGCTTTCCGACACTGCCCATCTGAACGA |
| | | | GACCCACTGCGCCCGCTGCCTGCAGCCCTACCAGCTGC |
| | | | TTGTGAATAGCAAAAGGCAGTGCCTGGAATGTGGCCTC |
| | | | TTCACCTGCAAAAGCTGTGGCCGCGTCCACCCGG |
| 188 | MLPH | 2534271 | AGTCGTGAAGATCGGCTCACTGGAGTGGTACTATGAGC |
| | | | ATGTGAAAGCCCGCTTCAAGAGGTTCGGAAGTGCCAAG |
| | | | GTCATCCGGTCCCT |
| 189 | MLPH | 2534272 | AGTGGAGAGTAAGAACGGCTTTTTGTTCCCAGGCATTTT |
| | | | AGGAATATTAA |
| 190 | MLPH | 2534278 | ATGGGTGGGTAGGTGAATACATGGATGGATGAGCCACT |
| | | | GATTGAGTGGGTGGATGGGTGGATGAATAGATGGGTGG |
| | | | AGGATAGATAGGTGGGTGTATGGGTGGGTGGATGGATT |
| | | | GATGCATGGATGGATGGGCTGCCCATTGAGTAGGTGGA |
| | | | TGAGTGGATAAATGGGTGGGTGGGTAGGTGAATAGATG |
| | | | AATAGATTGATAAATAGGGGGATGGGTGGATTGGTAGA |
| | | | TGGGTAGATGGAGGGATACATTGCTGTGTGGATAGGTG |
| | | | GGTGAA |
| 191 | MLPH | 2534279 | GGATGGGTGGATGGGCTGACAAATGGC |
| 192 | MLPH | 2534280 | CTGGGCCTGAACTGATATCTGAAGAGAGAAGTGGAGAC |
| | | | AGCGACCAGACAGATGAGGATGGAGAACCTGGCTCAG |
| | | | AGGCCCA |
| 193 | MLPH | 2534281 | AAAAAGCGCCTCCTCTCCGTCCACGACTTCGACTTCGA |
| | | | GGGAGACTCAGATGACTCCACTC |
| 194 | MLPH | 2534288 | TCTGGGTGCCACTCCCATCCGGAAGAGCAGCCGACCAG |
| | | | CATCTCACCTTCCAGACACGGCGCCCTGGCT |
| 195 | MLPH | 2534290 | GGTCGAATGTCATCAGGAATGAGCAGCTGCCCCT |
| 196 | MLPH | 2534291 | CCGATGTGGACACCTCTGATGAGGAAAGCATCCGGGCT |
| | | | CACGTGATGGCCTCCCACCATTCCAAGCGGA |
| 197 | MLPH | 2534294 | ATCTTTGAGCTGAATAAGCATATTTCAGCTGTGGAATGC |
| | | | CTGCTGACCTACCTGGAGAACACAGTTGTGCCTCCCTTG |
| | | | GC |
| 198 | MLPH | 2534296 | GTCATTCCCGATCTTTCCACCGAGGGCCTCTGTGATTTG |
| | | | GGGGCTTTGTCAGGAAAGTGGAGCCTCACGGAAAAGCA |
| | | | TACTGGCTAAAACACGCGGCTTCTTCATCGACTCAATCT |
| | | | AATCATCCCCTTGGTGTTCGTCTGTGAGACCCCAGGCAG |
| | | | CCAGCCCTGTCGATCTGTCTCAATAGGCTTC |
| 199 | MLPH | 2534299 | GTGCTGGAGTGCGCACGGAGGCCGATGTAG |
| 200 | MLPH | 2534300 | AGGAGGAGGCCCTGAGGAGGAAGCTGGAGGAGCTGAC |
| | | | CAGCAACGTCA |
| 201 | MLPH | 2534301 | CAGGAGACCTCGTCCGAGGAGGAGGAAGCCAAGGACG |
| | | | AAAAGGCAGAGCCCAACAGGGACAAATCAGTTGGGCC |
| | | | TCTC |
| 202 | MLPH | 2534302 | GGCACGGCTGCCCATCAAACCAACAGACAG |
| 203 | MLPH | 2534303 | CTGGGGACCCCGTCCAGTACAACAGGACCACAGATGAG |
| | | | GAGCTGTCAGAGCTGGAGGACAGAGTGGCAGTGACGG |
| | | | CCTCAGAAGTCCA |
| 204 | MLPH | 2534304 | ATTGAATCCAGGATTGCAGCCCTGAGGGCCGCAGGGCT |
| | | | CACGGTGAAGCCCTCGGGAAAGCC |
| 205 | MLPH | 2534305 | TCTTTATGAGGGGACTCTGAGCCTCTGCTCTGAGGATCT |
| | | | GAAACACACACACCCTGACAGTGTAAAATCCAAAAGG |
| | | | AGCCGCCTGAATCATGTTGCCTCATGTGGAAATCCTTAG |
| | | | TCCGCCGCCACGTG |
| 206 | MLPH | 2534311 | AAGACCACACACCAGTGCAGTGTGATGGGCCTTTCTGC |
| | | | TGCTTCATTAGTGTGAGGATTTCCAGGGCCACAGTGAG |
| | | | GAAGAATGTTAATGCCAGTGCCAGAGCAAAGGAGAAA |
| | | | GAAGTTGGCAAAACTGTTGATTTGCATGACAGCTGAAA |
| | | | TGTAAATACTTTTTAAAAAATATGTGATGTGGAAGCTTC |
| | | | TTAAAAGGGGATATGTCCATTTTTTTCTACCTTTTAAAT |
| | | | TTCTGAGGAGGCCAAGGCACTTGTTTGGGCTAAGTATG |
| | | | TGATTGATAAAGCACCATCCC |
| 207 | MLPH | 2534312 | ATGATGATTCTTTTGATCGGAAATCAGTGTACCGAGGCT |
| | | | CGCTGACACAGAGAAACCC |
| 208 | MLPH | 2534313 | AGGAGTGGCCCCAACCAATGTGATCAGTCGCAGGAGGC |
| | | | AACCAATCAGAGGCTGAAGGGAAGTTACAAAGTTACAC |
| | | | ATGAAGACTTGGCCGATGACCAG |
| 209 | MLPH | 2534314 | AAACCTGTGGTGGCCCACCAGTCCT |
| 210 | MLPH | 2534315 | ACAGAGCAGCCCTGCACTGTTTTCCCTCCACCACAGCC |
| | | | ATCCTGTCCCTCATTGGCTCTGTGCTTTCCACTATACAC |
| 211 | MLPH | 2534316 | CACCTGCAAGTGGACAGCGACATTCAGTCCTGCACTGC |
| | | | TCACCTGGGTTTACTGATGACTCCTGGCTGCCCCACCAT |
| | | | CCTCTCTGATCTGTGAGAAACAGCTAAGCTGCTGTGACT |
| | | | TCCCTTTAGGACAATGTTGTGTAAATCTTTGAAGGACAC |
| | | | ACCGAAGACCTTTATACTGTGATCTTTTACCCCTTTCAC |
| | | | TCTTGGCTTTCTTATGTTGCTTTCATGAATGGAATGGAA |
| | | | AAAAGATGACTCAGTTAAGGCACCAGCCATATGTGTAT |
| | | | TCTTGATGGTCTATATCGGGGTGTGAGCAGATGTTTGCG |
| | | | TATTTCTTGTGGGTGTGACTGGATATTAGACATCCGGAC |
| | | | AAGTGACTGAACTAATGATCTGCTGAATAATGAAGGAG |
| | | | GAATAGACACCCCAGTCCCCACCCTACGTGCACCCGCT |
| | | | CTGCAAGTTCCCATGTGATCTGTAGACCAGGGGAAATT |
| | | | ACACTGCGGTCAAGGGCAGAGCCTGCACATGACAGCAA |
| | | | GTGAGCATTTGATAGATGCTCAGATGCTAGTGCAGAGA |
| | | | GCCTGCTGGGAGACGAAGAGACAGCAGGCAGAGCTCC |
| | | | AGATGGGCAAGGAAGAGGCTTGGTTCTAGCCTGGCTCT |
| | | | GCCCCTCACTGCAGTGGATCCAGTGGGGCAGAGGACAG |
| | | | AGGGTCACAACCAATGAGGGATGTCTGCCAAGGATGGG |
| | | | GGTGCAGAGGCCACAGGAGTCAGCTTGCCACTCGCCCA |
| | | | TTGGTTACATAGATGATCTCTCAGACAGGCTGGGACTC |
| | | | AGAGTTATTTCCTAGTATCGGTGTGCCCCATCCAGTTTT |
| | | | AAGTGGAGCCCTCCAAGACTCTCCAGAGCTGCCTTTGA |
| | | | ACATCCTAACAGTAATCACATCTCACCCTCCCTGAGGTT |
| | | | CACTTTAGACAGGACCCAATGGCTGCACTGCCTTTGTCA |
| | | | GAGGGGGTGCTGAGAGGAGTGGCTTCTTTTAGAATCAA |
| | | | ACAGTAGAGACAAGAGTCAAGCCTTGTGTCTTCAAGCA |
| | | | TTGACCAAGTTAAGTGTTTCCTTCCCTCTCTCAATAAGA |
| | | | CACTTCCAGGAGCTTTCCAATCTCTCACTTAAAACTAAG |
| | | | GTTTGAATCTCAAAGTGTTGCTGGGAGGCTGATACTCCT |
| | | | GCAACTTCAGGAGACCTGTGAGCACACATTAGCAGCTG |
| | | | TTTC |
| 212 | GPR160 | 2651840 | CCAGGAGTGGAGCCCGGACGCCCGAGCCTTCCTGCTTC |
| | | | GGGATGGGGATTACCGCGGAGCCTTAGCAACTG |
| 213 | GPR160 | 2651841 | ATCCTGGCTGGCTCAAAATTCCCTCTAGATTACCTGCGA |
| | | | CCACCCCCAGGAACCCGGAGACTGAAACTCCT |
| 214 | GPR160 | 2651842 | CCTGCAGTCCGGAGACGAACGCACGGACCGGGCCTCCG |
| | | | GAGGCAGGTTCGGCTGGAAGGAACCGCTCTCGCTTCGT |
| | | | CCTACACTTGCGCAAATGTCTC |
| 215 | GPR160 | 2651850 | GCTTTGTGCAGTGGCCATTTCATAGCCAGTGAAGTTTAT |
| | | | CTGAGGCACTTGCTAATTGAAAACTTTTCTCAATACCCT |
| | | | GCCATGATGAAATATGGTTGGCACTGGCAATTTT |
| 216 | GPR160 | 2651851 | ATGTCCAAGTCCCGTGCGGCGGAGGCAGCAGCGGGGGT |
| | | | GACAGCGACGGCCCCGAGCCCGCAGATAGTGGAGCAG |
| | | | AGGGGTCCAGGGAGGCGCTGCACCGACGTTGGGGAGA |
| | | | AT |
| 217 | GPR160 | 2651852 | TTCAGGAGGAGAACTCCGTTACACGTCACGAA |
| 218 | GPR160 | 2651853 | AAAACCTCCGGCCTCGTCTTGTGATTCCACCAATGCAGC |
| | | | CATCGCCAAGCAAGCCTTGAAATAGCCCATCAAGGGCA |
| | | | AACAGGCCCCCGAAAAAAAGCTCAAGGAAAAGTCGCA |
| | | | AACTCACGGATTTCTACCCTGTCCCAAGGAGCTCCAGG |
| | | | AAGGGCAAAGCGGAGCTGCAATTTGAAGAAAGGGAAA |
| | | | GAATAGATGAATTGGTTGAAAGCGGAAAGGAAAGAAG |
| | | | GAGTGAAGACTGACCTCATCAATGACAAAGGCCGGGTG |
| | | | TGAACACCAGAGGGAGCTCTCCCGGGGCGCCTTTGTGG |
| | | | TAGAAAAGCACCGGAGCCTCACGGAGATCACCGACGCC |
| | | | ACGGGACCCCTCCATGGTGGATCTCCGTGGATCTCC |
| 219 | GPR160 | 2651854 | GTTGAACGTTTTGTGCTTCCCATGCATGC |
| 220 | GPR160 | 2651864 | TGCTCCACTTATCGGGCTCACCAAATACAGCTGCAGTAT |
| | | | GAATTCCATCTTCTACACAACAGTAAACCAGAATGTTC |
| | | | ATGTTCACAGCGTTCATCTCCCATCCTGTTGTCTAACGC |
| | | | ACATGGTTTTTTTAAACTTTTCTAAGATTGCATGAGATT |
| | | | CTGCAACACAACTGATTATAAAAACACTTGAAGTTTTT |
| | | | ACCTTTTTTTTTACTTTCCAACTCTCGTGAATGTACAGA |
| | | | GGACTTTCCA |
| 221 | GPR160 | 2651865 | CAGGGGAGAATAAGCTGAACGCAGCTGTTCTCTGACCT |
| | | | TGAGGCAGAGGGCAAGGAGTAGGTACAAGGACGTGTA |
| | | | GGAGAATTTATCTTAAATAGGCTTGTTCACTTGTGTTGT |
| | | | CCAGAAACGACTTTTGATCATCAGCGCGCATGACTGCT |
| | | | CCCTGAAAGGAAGAACAATAATGTTAATTACCCGCAGA |
| | | | CTGTGTTTGCTCCAGGCTTTCGGCATTATGTCTGTACTG |
| | | | AATAAAAGCAAGCAGCTCCAGCTGTTCGAGGCTGCTCT |
| | | | CTTCTTCAGCCATTAGTGCCGGGCAGCCC |
| 222 | GPR160 | 2651866 | AGCTTACTCACATAGCATATTGGTATATCAAAATGAAA |
| | | | TGCAAGGAACCAAAAATAACATAATTGAAGGCAGTAA |
| | | | AAGTGAAATTAAATAGGAAGATCATCAGTCAA |
| 223 | GPR160 | 2651869 | GAGCTTCAGGAAAAGACTTAATCTGAAGGATCCTGCAG |
| | | | CTAAAAAGCTTTGAAAACTGTGTTAAGGGGCCCCATAA |
| | | | GCATCGCTTCTAAACTTCACTGACAAAAGGGACTGGGG |
| | | | TCATGCTGTCTGGAGTCA |
| 224 | GPR160 | 2651870 | TGTATTTCAGCAGGTCTTCTTGAAA |
| 225 | GPR160 | 2651871 | TCCAGTTTTCCTGACAGCTTGTATAGATTATTGCCTGAA |
| | | | TTTCTCTAAAACAACCAAGCTTTCATTTAAGTGTCAAAA |
| | | | ATTATTTTATTTCTTTACAGTAATTTTAATTTGGATTTCA |
| | | | GTCCTTGCTTATGTTTTGGGAGACCCAGCCATCTACCAA |
| | | | AGCCTGAAGGCACAGAATGCTTATTCTCGTCACTGTCCT |
| | | | TTCTATGTCAGCATTCAGAGTTACTGGCTGTCATTTTTC |
| | | | ATGGTGATGATTTTATTTGTAGCTTTCATAACCTGTTGG |
| | | | GAAGAAGTTACTACTTTGGTACAGGCTATCAGGA |
| 226 | GPR160 | 2651872 | TGTTTTACTTAAAGTTCAGATTCCAGCATATATTGAGAT |
| | | | GAATATTCCCTGGTTATACTTTGTCAATAGTTTTCTC |
| 227 | GPR160 | 2651873 | AAAGACATTGGATTACCTTTGGATCCATTTGTCAACTGG |
| | | | AAGTGCTGCTTCATTCCACTTACAATTCCTAATCTTGAG |
| | | | CAAATTGAAAAGCCTATATCAATAATGATT |
| 228 | GPR160 | 2651874 | GTTACAGCTGTCATAAGATCATAATTTTATGAACAGAA |
| | | | AGAACTCAGGACATATTAAAAA |
| 229 | GPR160 | 2651875 | CCCTGACTGATAGCATTTCAGAATGTGTCTTTTGAAGGG |
| | | | CTATGATACCAGTTATTAAATAGTGTTTTATTTTAAAAA |
| | | | CAAAATAATTCCAAGAAGTTTTTATAGTTATTCAGGGA |
| | | | CACTATATTACAAATATTACTTTGTTATTAACACAAAAA |
| | | | GTGATAAGAGTTAACATTTGGCTATACTGATGTTTGTGT |
| | | | TACTCAAAAAAACTACTGGATGCAAACTGTTATGTAAA |
| | | | TCTGAGATTTCACTGACAACTTTA |
| 230 | CCNB1 | 2813417 | GTGCGGGGTTTAAATCTGAGGCTAG |
| 231 | CCNB1 | 2813418 | CTCTTCTCGGCGTGCTGCGGCGGAACGGCTGTTGGTTTC |
| | | | TGC |
| 232 | CCNB1 | 2813419 | GTAGGTCCTTGGCTGGTCGGGCCTCCGGTGTTCTGCTTC |
| | | | TCCCC |
| 233 | CCNB1 | 2813420 | AGCCGCTTCGGACTGCGAACTAACGCGGCCTTCTTAGC |
| | | | TGCTGCCTGCTCTCCCTGCCTCGCCTGCGGGAGCCTCCC |
| | | | GAGCGGGAGAGGGCCGCAGGAGCGATTTGGGGAGGAA |
| | | | GGTGGGAGGGGACTCACCAAGAGAGCGCCGAGGTGGG |
| 234 | CCNB1 | 2813421 | TGCTGAAAATAAGGCGAAGATCAACATGGCAGGCGCA |
| | | | AAGCGCGTTCCTACGGCCCCTGCTGCAACCTCCAAGCC |
| | | | CGGACTGAGGCCAAGAACAGCTCTTGGGGACATTGGTA |
| | | | ACAAAGTCAGTG |
| 235 | CCNB1 | 2813422 | AAGCAAAACCTTCAGCTACTGGAAAAGTCATTGATAAA |
| | | | AAACT |
| 236 | CCNB1 | 2813425 | ACTGGAAACATGAGAGCCATCCTAATTGACTGGCTAGT |
| | | | ACAGGTTCAAATGAAATTCAGGTTGTTGCAGGAGACCA |
| | | | TGTACATGACTGTCTCCATTATTGATCGGTTC |
| 237 | CCNB1 | 2813427 | TGTGACTGACAACACTTATACTAAGCACCAAATCAGAC |
| | | | AGATGGAAATGAAGATTCTAAGAGCTTTAAACTTTGGT |
| | | | CTGGGTCGGCCTCTACCTTTGCACTTCCTTCGGAGAGCA |
| | | | TCTAAGATTGG |
| 238 | CCNB1 | 2813429 | TGATGTCGAGCAACATACTTTGGCCAAATACCTGATGG |
| | | | AACTAACTATGTTGGACTATGACATGGTGC |
| 239 | CCNB1 | 2813432 | AACATTACCTGTCATATACTGAAGAATCTCTTCTTCCAG |
| | | | TTATGCAGCACCTGGCTAAGAATGTAGTCATGGTAAAT |
| | | | CAAGGACTTACAAAGC |
| 240 | CCNB1 | 2813434 | TCAAGAACAAGTATGCCACATCGAAGCATGCTAAGATC |
| | | | AGCACTCTACCACAGCTGAATTCTGCACTAGTTCAAG |
| 241 | CCNB1 | 2813435 | AAACTTGAGTTGGAGTACTATATTTACAA |
| 242 | CCNB1 | 2813436 | ATTACTGTTGCATTTACTTTTAATAAAGCTTGTGG |
| 243 | CCNB1 | 2813437 | CCTGGGGATCCAATTGATGTATATGTTTATATACTGGGT |
| | | | TCTTGTTTTATATACCTGGCTTTTACTTTATTAATATGAG |
| | | | TTACTGAAGGTGATGGAGGTATTTGAAAATTTTACTTCC |
| | | | ATAGGACATACTGCATGTAAGCCAAGTCATGGAGAATC |
| | | | TG |
| 244 | CXXC5 | 2831352 | CCCGGGCAGCGTTCATAGCTCCTGCCCGGGCGGGCGCG |
| | | | CGGCGGCGGCGGCAGAGGCGGCTGAGCCTGAGCGGGG |
| | | | ATGTAGAGGCGGCGGCAGCAGAGGCGGCACTGGCGGC |
| | | | AAGAGCAGACGCCCGAGCCGAGCGAGAAGAGCGGCAG |
| | | | AGCCTTATCCCCTGAAGCCGGGCCCCGCGTCCCAGCCC |
| | | | TGCCCAGCCCGCGCCCAGCCATGCGCGCCGCCTGCTGA |
| | | | GTCCGGGCGCCGCACGCTGAGCCCTCCGCCCGCGAGCC |
| | | | GCGCTCAGCTCGGGGGTGATTAGTTGCTTT |
| 245 | CXXC5 | 2831353 | AGTTCGCTGCAAGTCGGCGGAAAGTTTGGCTGCGCGGG |
| | | | TTCCCCCGAAGTTCA |
| 246 | CXXC5 | 2831354 | CCTCATCCTCGCAGTAGCTGGGTCTCTCCCAGGGACGCC |
| | | | CCTAGTCAGCCTTGG |
| 247 | CXXC5 | 2831356 | GTAATTATGGAATTTTGCTTGGGAAATTAATTTGAAAA |
| | | | AGTTAATTAATTGGTGGTTCCGGAGGTGGCGGGCTCCA |
| | | | CGCCCGGCCAGTCTTGCTGACGTCAGTGCTGACCCACT |
| | | | GGAGACGTGCAGCTTCCG |
| 248 | CXXC5 | 2831357 | GGGGTCTGTGACAGCTTGCCCCCAACCACGGAGAGG |
| 249 | CXXC5 | 2831359 | TTGTCCAGGGTGGTCTCAAAACTCC |
| 250 | CXXC5 | 2831360 | CTGAGCAGCGAGGCCCACCAGGCATCTCTGTTGTGGGC |
| | | | AGCAGGGCCAGGTCCTGGTCTGTGGACCCTCGGCAGTT |
| | | | GGCAGGCTCCCTCTG |
| 251 | CXXC5 | 2831361 | GCTCCCAGGATGCCGGCGGCAGTAGCAG |
| 252 | CXXC5 | 2831362 | CAGGAGCAGCAGACAAGAGTGCAGTGGTGGCTGCCGC |
| | | | CGCACCAG |
| 253 | CXXC5 | 2831363 | CCCGAGCGTCGGAACAAGAGCGGTATCATCAGTGAGCC |
| | | | CCTCAACAAGAGCCTGCGCCGCTCCCGC |
| 254 | CXXC5 | 2831364 | CCATGGCGGTGGACAAAAGCAACCCTACCTCAAAGCAC |
| | | | AAAAGTGGTGCTGTGGCCAGCCTGCTGAGCAAGGCAGA |
| | | | GCGGGCCACGGAGCTGGCAGCCGAGGGACAGCTGACG |
| | | | CTGCAGCAGTTTGCGCAGTCCACAGAGATGCTGAAGCG |
| | | | CGTGGTGCAGGAGCATCTCCCGCTGATGAGCGAGGCGG |
| | | | GTGCTGGCCTGCCTGACATGGAGGCTGTGGCAGGTGCC |
| | | | GAAGCCCTCAATGGCCAGTCCGACTTCCCCTACCTGGG |
| | | | CGCTTTCCCCATCAACCCAGGCCTCTTCATTATGACCCC |
| | | | GGCAGGTGTGTTCCTGGCCGAGAGCGCGCTGCACATGG |
| | | | CGGGCCTGGCTGAGTACCCCATGCAGGGAGAGCTGGCC |
| | | | TCTGCCATCAGCTCCGGCAAGAAGA |
| 255 | CXXC5 | 2831365 | TTCAGAAAATGTGAGGAACTCAAAAAGAAGCCTTCCGC |
| | | | TGCTCTGGA |
| 256 | CXXC5 | 2831367 | GTGATGCTTCCGACGGGAGCCGCCTTCCGGTGGTTTCA |
| | | | GTGA |
| 257 | CXXC5 | 2831368 | AATGTCACTGCTCGTGTGGTCTCCAGCAAGGGATTCGG |
| | | | GCGAAGACAAACGGATGCACCCGTCTTTAGAACCAAAA |
| | | | ATATTCTCTCACAGATTTCATTCCTGTTTTTATATATATA |
| | | | TTTTTTGTTGTCGTTTTAACATCTCCACGTCCCTAGCATA |
| | | | AAAAG |
| 258 | CXXC5 | 2831369 | ATCTATAAAGTACCGAGACTTCCTGGGCAAAGAATGGA |
| | | | CAATCAGTTTCCTTCCTGTGTCGATGTCGATGTTGTCTG |
| | | | TGCAGGAGATGCAGTTTTTGTGTAGAGAATGTAAATTTT |
| | | | CTGTAACCTTTTGAAATCTAGTTACTAATAAGCACTACT |
| | | | GTAATTTAGCACAGTTTAACTCCACCCTCATTTAAACTT |
| | | | CCTTTGATTCTTTCCGACCATGAAATAG |
| 259 | CXXC5 | 2831370 | CCTGGAGAATCCACTCACGTTCATAAAGAGAATGTTGA |
| | | | TGGCGCCGTGTAGAAGCCGCTCTGTATCCATCCACGCG |
| | | | TGCAGAGC |
| 260 | PTTG1 | 2838203 | GGCTTAGATGGCTCCGAGCCCGTTTGAGCGTGGTCTCG |
| | | | GACTGCTAACTGGACCAACGGCAACTGTCTGATGAGTG |
| | | | CCAGCCCCAAACCGCGCGCTGCTCGGGACCTTAGAGCC |
| | | | TCTG |
| 261 | PTTG1 | 2838204 | TGTTCCGCTGTTTAGCTCTTGTTTTTTGTGTGGACACTCC |
| | | | TAGGATAGAAAGTTTGGTATGTTGCTATACCTTTGCTTC |
| 262 | PTTG1 | 2838205 | GCACCCGTGTGGTTGCTAAGGATGGGC |
| 263 | PTTG1 | 2838206 | TATACAAGGCTGCAGTCGGATACACTGGTATTGTGGAC |
| | | | GTGGCCTGGAGCTGGACGAGACATTTAGTGTACTTTTTG |
| | | | GGCAATTGGAGTCGTTTGTTATTGGTCCTTTTTCATTTTT |
| | | | AATATCTTAATGAGATGATTTAAGGAAGTTACTGAATC |
| | | | TCTGCTATTAGGCCTATC |
| 264 | PTTG1 | 2838207 | GATCTCAAGTTTCAACACCACGTTTTGGCAAAACGTTCG |
| | | | ATGCCCCACCAGCC |
| 265 | PTTG1 | 2838208 | GTAAGTGTTGGCTATAAAGACACTGTTTAAACACTTAA |
| | | | GCACTTTTGACTCTTAAAATGACTATTGGCATCATCCTA |
| | | | CGTAGCTTTCTTC |
| 266 | PTTG1 | 2838209 | CTGCCTCAGATGATGCCTATCCAGAAATAGAAAAATTC |
| | | | TTTCCCTTCAATCCTCTAG |
| 267 | PTTG1 | 2838210 | ATGAGACTGTCTGAATCTGGGTTGCTTTGGACAAGTGT |
| | | | ACTTGTTGATGGAATTATTTGCAAGGTATCATCTTAGGT |
| | | | CAGGAGGGGAATAGGAACAAAGATGTAGAAGACATTG |
| | | | TTCCTGTCTGTAAAAGCTTATCACCTAGAGGAGGTAAG |
| | | | ATGTATTCATGAACATTGAATAAGTCCCATTGTGGACA |
| | | | GTCTTTCTCACAAGGCTT |
| 268 | PTTG1 | 2838211 | CTGAAGAGCACCAGATTGCGCACCTCCCCTTGAGTGGA |
| | | | GTGCCTCTCATGATCCTTGACGAGGAGAGAGA |
| 269 | PTTG1 | 2838212 | CTGTTGCAGTCTCCTTCAAGCATTCTGTCGACCCTGGAT |
| | | | GTTGAATTGCCACCTGTTTGCTGTGACATAGATAT |
| 270 | PTTG1 | 2838213 | TCTTAGTGCTTCAGAGTTTGTGTGTATTTGT |
| 271 | FGFR4 | 2842913 | CCGCCGTCGCGGGTACATTCCTCGCTCCCGGCCGAGGA |
| | | | GCGCTCGGGCTGTCTGCGGACCCTGCCGCGTGCAGGGG |
| | | | TCGCGG |
| 272 | FGFR4 | 2842915 | GGCAGTTGGTGGGAAGTCCAGCTTGGGTCCCTGAGAGC |
| | | | T |
| 273 | FGFR4 | 2842916 | CCCTGTTGGGGGTCCTGCTGAGTGTGCCTGGGCCTCCAG |
| | | | TCTTGTCCCTGGAGGCCTCTGAGGAAGTGGA |
| 274 | FGFR4 | 2842918 | TACAAGGAGGGCAGTCGCCTGGCACCTGCTGGCCGTGT |
| | | | ACGGGGCTGGAGGGGCCGCCTAGAGATTGCCAGCTTCC |
| | | | TACCTGAGGATGCTGGCCGCTACCTCTGCCTGGCACGA |
| | | | GGCTCCATGATCGTCCTGCAGA |
| 275 | FGFR4 | 2842920 | TTGACCTCCAGCAACGATGATGAGGACCCCAAGTCCCA |
| | | | TAGGGACCCCTCGAATAGGCACAGTTACCCCCAGCAA |
| 276 | FGFR4 | 2842921 | GCTGCTCATCTGATCACTGAGAAGAGGAGGCCTGTGTG |
| | | | GGAACACACGGTCATTCTAGGGGCCTTCC |
| 277 | FGFR4 | 2842922 | CTGCATGCAGTACCTGCGGGGAACACCGTCAAGTTCCG |
| | | | CTGTCCAGCTGCAGGCAACCCCACGCCCACCATCCGCT |
| | | | GGCTTAAGGATGGACAGGCCTTTCATGGGGAGAACC |
| 278 | FGFR4 | 2842923 | CTGCGCCATCAGCACTGGAGTCTCGTG |
| 279 | FGFR4 | 2842924 | ACATACACCTGCCTGGTAGAGAACGCTGTGGGCAGCAT |
| | | | CCGCTATAACTACCTGCTAGAT |
| 280 | FGFR4 | 2842926 | CGGCCAACACCACAGCCGTGGTGGGCAGCGACGTGGA |
| | | | GCTGCTGTGCAAGGTGTACAGCGATGCCCAGCCCCACA |
| | | | TCCAGTGGCTGAAGCACATCGTCATCAACGGCAGCAGC |
| | | | TTCGGAGCCGACGGTTTCC |
| 281 | FGFR4 | 2842928 | AGGTCCTGTACCTGCGGAACGTGTCAGCCGAGGACGCA |
| | | | GGCGAGTACACCTGCCTCGCAGGCAATTCCATCGGCCT |
| | | | CTCCTACCAGTCTG |
| 282 | FGFR4 | 2842929 | AGGAGATGCTGCGAGATGCCCCTCTGGGCC |
| 283 | FGFR4 | 2842930 | GCCCGAGGCCAGGTATACGGACATCATCCTGTACGCGT |
| | | | CGGGCTCCCTGGCCTTGGCTGTGCTCCTGCTGCTGGCCG |
| | | | GGCTGTATCGAGGGCAGGCGCTCCACG |
| 284 | FGFR4 | 2842931 | GGCGCATCCCCCACCTCACATGTGACAGCCTGACTCCA |
| | | | GCAGGCAGAACCAAGTCT |
| 285 | FGFR4 | 2842932 | TTCCGGCAAGTCAAGCTCATCCCTGGTACGAGGCGTGC |
| | | | GTCTCTCCTCCAGCGGCCCCGCCTTGCTCGCCGGCCTCG |
| | | | TGAGTCTAGATCTACCTCTCGACCCACTATGGGAGTTC |
| 286 | FGFR4 | 2842933 | CCCTAGGCGAGGGCTGCTTTGGCCAGGTAGTACGTGCA |
| | | | GAGGCCTTTG |
| 287 | FGFR4 | 2842934 | AGGTGATGAAGCTGATCGGCCGACACAAGAACATCATC |
| | | | A |
| 288 | FGFR4 | 2842935 | CCCTGTACGTGATCGTGGAGTGCGCCGCCAAGGGAAAC |
| | | | CTGCGGGAGTTCCTGCGGGCCCGGCGCCCCCCAGGCCC |
| | | | CGACCTCAGCCCCGACGGTCCTCGGAGCAGTGAGGGGC |
| 289 | FGFR4 | 2842936 | CGGCGTCCACCACATTGACTACTATAAGAAAA |
| 290 | FGFR4 | 2842938 | GGCCTTGTTTGACCGGGTGTACACACACCAG |
| 291 | FGFR4 | 2842939 | TCGGGGGCTCCCCGTATCCTGGCATCCCGGTGGAGGAG |
| | | | CTGTTCTCGCTGCTGCGGGAGGGACATCGGATGGACCG |
| | | | ACCCCCACACTGC |
| 292 | FGFR4 | 2842940 | CTGAGGCTCCCTGTGACCCTCCGCCC |
| 293 | FGFR4 | 2842941 | ACGGGCTGATGCGTGAGTGCTGGCACGCAGCGCCCTCC |
| 294 | FGFR4 | 2842943 | CTCGACCTCCGCCTGACCTTCGGACCCTATTCCCCCTCT |
| | | | GGTGGGGACGCCAGCAGCACCTGCTCCTCCAGCGATTC |
| | | | TGTCTTCAGCCACGACCCCCTGCCATTGGGATC |
| 295 | FGFR4 | 2842944 | CCTGACACAGTGCTCGACCTTGATAGCATGGGGCCCCT |
| | | | GGCCCAGAGTTGCTGTGCCGTGTCCAAGGGCCGTGCCC |
| | | | TTGCCCTTGGAGCTGCCGTGCCTGTGTCCTGATGGCCCA |
| | | | AATGTCAGGGTTCTGCTCGGCTTCTTGGACCTTGGCGCT |
| | | | TAGTCCCCATCCCGGGTTTGGCTGAGCCTGGCTGGAGA |
| | | | GCTGCTATGCTAAACCTCCTGCC |
| 296 | FOXC1 | 2891769 | AGAAGGGCGCCTGCTTGTTCTTTCTTTTTGTCTGCTTTCC |
| | | | CCCGTTTGCGCCTGGAAGCTGCGCCGCGAGTTCCTGCA |
| | | | AGGCGGTCTGCCGCGGCCGGGCCCGGCCTTCTCCCCTC |
| | | | GCAGCGACCCCGCCTCGCGGCCGCGCGGGCCCCGAGGT |
| | | | AGCCCGAGGCGCCGGAGGAGCCAGCCCCAGCGAGCGC |
| | | | CGGGAGAGGCGGCAGCGCAGCCGGACGCACAGCGCAG |
| | | | C |
| 297 | FOXC1 | 2891770 | ATGCAGGCGCGCTACTCCGTGTCCAGCCCCAACTCCCT |
| | | | GGGAGTGGTGCCCTACCTCGGCGGCGAGCAGAGCTACT |
| | | | ACCGCGCGGCGGCCGCGGCGGCCGGGGGCGGCTACAC |
| | | | CGCCATGCCGGCC |
| 298 | FOXC1 | 2891771 | ATGAGCGTGTACTCGCACCCTGCGCACGCCGAGCAGTA |
| | | | CCCGGGCGGCATGGCCCGCGCCTACGGGCCCTACACGC |
| | | | CGCAGCCGCAGCCCAAGGACATGGTGAAGCCGCCCTAT |
| | | | AGCTACATCGCGCTCATCACCATGGCCATCCAGAACGC |
| | | | CCCGGACAAGAAGATCACCCTGAAC |
| 299 | FOXC1 | 2891772 | AGGACAGGCTGCACCTCAAGGAGCCGCCCCCG |
| 300 | FOXC1 | 2891773 | GCCGACGGCAACGCGCCCGGTCCGCA |
| 301 | FOXC1 | 2891774 | GCGCATCCAGGACATCAAGACCGAGAACG |
| 302 | FOXC1 | 2891775 | CGCCGCGGTGCCCAAGATCGAGAGCCCCGACAGCAGCA |
| | | | GCAGCAGCCTGTCCAGCGGGAGCAGCCCCCCGGGCAGC |
| | | | CTGCCGTCGGCGCGGCCGCTCAG |
| 303 | FOXC1 | 2891776 | CCGCCGCCGCACCATAGCCAGGGCTTCAGCGTGGACAA |
| | | | CATCATGACGTCGCTGCGGGGGTCGCCGCAGAGCGCGG |
| | | | CCGCGGAGCTCAGCTCCGGCCTTCTGGCCTCGGCGGCC |
| 304 | FOXC1 | 2891777 | GTCCTCGCGCGCGGGGATCGCACCCCCGCTGGCGCTCG |
| | | | GCGCCTACTCGCCCGGCCAGAGCTCCCTCTACAGCTCCC |
| | | | CCTGCAGCCAGACCTCCAGCGCGGGCAGCTCGGGCGGC |
| | | | GGCGGCGGCGGCGCGGGGGCCGCGGGGGGCGCGGGCG |
| | | | GCGCCGGGACCTACCACTGCAACCT |
| 305 | FOXC1 | 2891778 | ATGAGCCTGTACGCGGCCGGCGAGCGCGGGGGCCACTT |
| | | | GCAGGGCGCGCCCGGGGGCGCGGGCGGCTCGGCCGTG |
| | | | GACGACCCCCTGCCCGACTACTCTCTGCCTCCGGT |
| 306 | FOXC1 | 2891779 | GGCCGGCCACCACCCTGCGGCCCACCAAGGCCGCCTCA |
| | | | C |
| 307 | FOXC1 | 2891780 | ACCTGGGCCACTTGGCGAGCGCGGCGG |
| 308 | FOXC1 | 2891781 | CAGAACTTCCACTCGGTGCGGGAGATGTTCGAGTCACA |
| | | | GAGGATCGGCTTGAACAACTCTCCAGTGAACGGGAATA |
| | | | GTAGCTGTCAAATGGCCTTCCCTTCCAGCCAGTCTCTGT |
| | | | ACCGCACGTCCGGAGCTTTCGTCTACGA |
| 309 | FOXC1 | 2891782 | TGAGAATATTCACCACACCAGCGAACAGAATATCCCTC |
| | | | CAAAAATTCAGCTCACCAGCACCAGCACGAAGAAAACT |
| | | | CTATTTTCTTAACCGATTAATTCAGAGCCACCTCCACTT |
| | | | TGCCTTGTCTAAATAAACAAACCCGTAAACTGTTTTATA |
| | | | CAGAGACAGCAAAATCTTGGTTTATTAAAGGACAGTGT |
| | | | TACTCCAGATAACACGTAAGTTTCTTCTTGCTTTTCAGA |
| | | | GACCTGCTTTCCCCTCCTCCCGTCTCCCCTCTCTTGCCTT |
| | | | CTTCCTTGCCTCTCACCTGTAAGATATTATTTTATCCTAT |
| | | | GTTGAAGGGAGGGGGAAAGTCCCCGTTTATGAAAGTCG |
| | | | CTTTCTTTTTATTCATGGACTTGTTTTAAAATGTAAATTG |
| | | | CAACATAGTAATTTATTTTTAATTTGTAGTTGGATGTCG |
| | | | TGGACCAAACGCCAGAAAGTG |
| 310 | FOXC1 | 2891783 | GGAGAAACCCTCTGACTAGTCCATGTCAAATTTTACTA |
| | | | AAAGTCTTTTTGTTTAGATTTATTTTCCTGCAGCATCTTC |
| | | | TGCAAAATGTACTATATAGTCAGCTTGCTTTGAGGCTAG |
| | | | TAAAAAGATATTTTTCTAAACAGATTGGAGTTGGCATA |
| | | | TAAACAAATACGTTTTCTCACTAATGACAGTCCATGATT |
| | | | CGGAAATTTTAAGCCCATGAATCAGCCGCGGTCTTACC |
| | | | ACGGTGATGCCTGTGTGCCGAGAGATGGGACTGTGCGG |
| | | | CCAGATATGCACAGATAAATATTTGGCTTGTGTATTCCA |
| | | | TATAAAATTGCAGTGCATATTATACATCCCTGTGAGCCA |
| | | | GATGCTGAATAGATATTTTCCTATTATTTCAGTCCTTTA |
| | | | TAAAAGGAAAAATAAACCAGTTTTTAAATGTATGTATA |
| | | | TAATTCTCCCCCATTTACAATCCTTCATGTATTACATAG |
| | | | AAGGATTGCTTTTTTAAAAATATACTGCGGGTTGGAAA |
| | | | GGGATATTTAATCTTTGAGAAACTATTTTAGAAAATATG |
| | | | TTTGTAGAACAATTATTTTTGAAAAAGATTTAAAGCAAT |
| | | | AACAAGAAGGAAGGCGAGAGGAGCAGAACATTTTGGT |
| | | | CTAGGGTGGTTTCTTTTTAAACCATTTTTTCTTGTTAATT |
| | | | TACAGTTAAACCTAGGGGACAATCCGGATTGGCCCTCC |
| | | | CCCTTTTGTAAATAACCCAGGAAATGTAATAAATTCATT |
| | | | ATCTTAGGGTGATCTGCCCTGCCAATCAGACTTTGGGG |
| | | | AGATGGCGATTTGATTACAGACGTTCGGGGGGGTGGGG |
| | | | GGCTTGCAGTTTGTTTTGGAGATAATACAGTTTCCTGCT |
| | | | ATCTGCCGCTCCTATCTAGAGG |
| 311 | ESR1 | 2931764 | AGAAGCTCTTTAACAGGCTCGAAAGGTCCATGCTCCTTT |
| | | | CTCCTGCCCATTCTATAGCATAAG |
| 312 | ESR1 | 2931765 | CAAAGATCTCTTCACATTCTCCGGGACTGCGGTACCAA |
| | | | ATATCAGCACAGCACTTCTTGAAAAAGGATGTAGATTT |
| | | | TAATCTGAACTT |
| 313 | ESR1 | 2931775 | ACGAAGTGGAGGAGTATTACATTTCAGCTGGAAACACA |
| | | | TCCCTAGAATGCCAAAACATTTATTCCAAAGTCTGGTTT |
| | | | CCTGGTGCAATCGGAGGCATGGCAATGCCTCTGTTCAG |
| | | | AGA |
| 314 | ESR1 | 2931776 | AAGCATAGGGTACTTTCCAGCCTCCAAGGGTAGGGGCA |
| | | | AAGGGGCTGGGGTTTCTCCTCCCCAGTACAGCTTTCTCT |
| | | | GGCTGTGCCACACTGCTCCCTGTGAGCAGACAGCAAGT |
| | | | CTCCCCTCACTCCCCACTGCCATTCATCCAGCGCTGTGC |
| | | | AGTAGCCCAGCTGCGTGTCTGCCGGGAGGGGCTGCCAA |
| | | | GTGCCCTGCCTACTGGCTGCTTC |
| 315 | ESR1 | 2931779 | GCAGCACATTAGAGAAAGCCGGCCCCTGGATCCGTCTT |
| | | | TCGCGTTTATTTTAAGCCCAGTCTTCCCTGGGCCACCTT |
| | | | TAGCAGATCCTCGTGCGCCCCCGCCCCCTGGCCGTGAA |
| | | | ACTCAGCCTCTATCCAGCAGCGACGACAAGTA |
| 316 | ESR1 | 2931780 | CCAATGTCAGGGCAAGGCAACAGTCCCTGGCCGTCCTC |
| | | | CAGCACCTTTGTAATGCATATGAGCTCGGGAGACCAGT |
| | | | ACTTAAAGTTGGAGGCCCGGGAGCCCAGGAGCTGGCGG |
| | | | AGGGCGTTCGTCCTGGGACTGCACTTGCTCCCGTCGGGT |
| | | | CGCCCGGCTTCACCGGACCCGCAGGCTCCCGGGGCAGG |
| | | | GCCGGGGCCAGAGCTCGCGTGTCGGCGGGACATGCGCT |
| | | | GCGTCGCCTCTAACCTCGGGCTGTGCT |
| 317 | ESR1 | 2931781 | GTGGCCCGCCGGTTTCTGAGCCTTCTGCCCTGCGGGGAC |
| | | | ACGGTCTGCACCCTGCCCGCGGCCACGGACC |
| 318 | ESR1 | 2931782 | TCTGGGATGGCCCTACTGCATCAGATCCAAGGGAACGA |
| | | | GCTGGAGCCC |
| 319 | ESR1 | 2931783 | CCCCTGGGCGAGGTGTACCTGGACAGCAGCAAGCCCGC |
| | | | CGTGTACAACTACCCCGAGGGCGCCGCCTACGAGTTCA |
| | | | ACGCCGCGGCCGCCGCCAACGCGCAGGTCTACGGTCAG |
| 320 | ESR1 | 2931784 | TGAGGCTGCGGCGTTCGGCTCCAACGGCCTGGGGGGTT |
| | | | TCCCCCCACTCAACAGCGTGTCTCCGAGCCCGCTGATGC |
| | | | TACTGCACCCGCCGCCGCAGCTGTCGCCTTTCCTGCAGC |
| | | | CCCACGGCCAGCAGGTGCCCTACTACCTGGAGAACGAG |
| | | | C |
| 321 | ESR1 | 2931791 | GTGCTGTCATGTGGACTGTCCTCCCGAGTGTCCCACTGG |
| | | | ATGTTCAGAGAATTTATGTGAAGGTCACGTCATTTAGC |
| | | | ATTGAGATGCTGTGGTTACCTTCTTCCATTTCTTCCATA |
| | | | ATATGCAGCCACATCTATGTGTGAAGAAATGTAATAGA |
| | | | TAAAATTTCTCTGGACGCATAATAATGTGAGAAAGATT |
| | | | GTCACATGTCCCAGCAA |
| 322 | ESR1 | 2931798 | CAAATTCAGATAATCGACGCCAGGGTGGCAGAGAAAG |
| | | | ATTGGCCAGTACCAATGACAAGGGA |
| 323 | ESR1 | 2931799 | AATCTGCCAAGGAGACTCGCTACTGTGCAGTGTGCAAT |
| | | | GACTATGCTTCAGGCTACCATTATGGAGTCTGGTCCTGT |
| 324 | ESR1 | 2931812 | CAGGCCTGCCGGCTCCGTAAATGCTACGAAGTGGGAAT |
| | | | GA |
| 325 | ESR1 | 2931821 | AACCTTTGGCCAAGCCCGCTCATGATCAAACGCTCTAA |
| | | | GAAGAACAGCCTGGCCTTGTCCCTGACGGCCGACCAGA |
| | | | TGGTCAGTGCCTTGTTGGATGCTGAGCCCCCGATACTCT |
| | | | ATTCCGAGTATGATCCTACCAGACCCTTCAGTGAAGCTT |
| | | | CGATGATGGGCTTACTGACCAACCTGGCAGA |
| 326 | ESR1 | 2931835 | TGGATATATGTGTGATCCTGGGTGTGCCAAATGCTGTG |
| | | | GCTTCCTGAAGCTTAGATTTCCAGCTTGTCACCTTCAAG |
| | | | GTTACCTTGTGAATAGGAC |
| 327 | ESR1 | 2931836 | TGACTATGGATTTTGCCTGTTGCTTTGTTTCCACCAACT |
| | | | CTCCCTGAAGATGAGGCGCACAGACAGACAACTCACAG |
| | | | GCAAGAACAGCCTGGTCCATCTTGAAAGATTCTC |
| 328 | ESR1 | 2931839 | GCTTTGTGGATTTGACCCTCCATGATCAG |
| 329 | ESR1 | 2931840 | GTCCACCTTCTAGAATGTGCCTGGCTAGAGATCCTGATG |
| | | | ATTGGTCTCGTCTGGCGCTCCATGGAGCACCCAGGGAA |
| | | | GCTACTGTTTGCTCCTA |
| 330 | ESR1 | 2931850 | TGGAGATCTTCGACATGCTGCTGGCTACATCATCTCGGT |
| | | | TCCGCATGATGAATCTGCAGGGAGAGGAGTTTGTGTG |
| 331 | ESR1 | 2931859 | GAGAAGGACCATATCCACCGAGTCCTGGACAAGATCAC |
| | | | AGA |
| 332 | ESR1 | 2931861 | CATGAAGTGCAAGAACGTGGTGCCCCTCTATGACCTGC |
| | | | TGCTGGAGATGCTGGACGCCCACCGCCTACATGCGCCC |
| | | | ACTAGCCGTGGAGGGGCATCCGTGGAGGAGACGGACC |
| | | | AAAGCCACTTGGCCACTGCGGGCTCTACTTCATCGCATT |
| | | | CCTTG |
| 333 | ESR1 | 2931862 | CCTGGCTCCCACACGGTTCAGATAATCCCTGCTGCATTT |
| | | | TACCCTCATCATGCACCACTTTAGCCAAATTCTGTCTCC |
| | | | TGCATACACTCCGGCATGCATCCAACACCAATGGCTTTC |
| | | | TAGATGAGTGGCCATTCATTTGCTTGCTCAGTTCTTAGT |
| | | | GGCACATCTTCTGTCTTCTGTTGGGAACAGCCAAAGGG |
| | | | ATTCCAAGGCTAAATCTTTGTAACAGCTCTCTTTCCCCC |
| | | | TTGCTATGTTACTAAGCGTGAGGATTCCCGTAGCTCTTC |
| | | | ACAGCTGAACTCAGTCTATGGGTTGGGGCTCAGATAAC |
| | | | TCTGTGCATTTAAGCTACTTGTAGAGACCCAGGCCTGG |
| | | | AGAGTAGACATTTTGCCTCTGATAAGCACTTTTTAAATG |
| | | | GCTCTAAGAATAAGCCACAGCAAAGAATTTAAAGTGGC |
| | | | TCCTTTAATTGGTGACTTGGAGAAAGCTAGGTCAAGGG |
| | | | TTTATTATAGCACCCTCTTGTATTCCTATGGCAATGCAT |
| | | | CCTTTTATGAAAGTGGTACACCTTAAAGCTTTTATATGA |
| | | | CTGTAGCAGAGTATCTGGTGATTGTCAATTCATTCCCCC |
| | | | TATAGGAATACAAGGGGCACACAGGGAAGGCAGATCC |
| | | | CCTAGTTGGCAAGACTATTTTAACTTGATACACTGCAGA |
| | | | TTCAGATGTGCTGAAAGCTCTGCCTCTGGCTTTCCGGTC |
| | | | ATGGGTTCCAGTTAATTCATGCCTCCCATGGACCTATGG |
| | | | AGAGCAGCAAGTTGATCTTAGTTAAGTCTCCCTATATG |
| | | | AGGGATAAGTTCCTGATTTTTGTTTTTATTTTTGTGTTAC |
| | | | AAAAGAAAGCCCTCCCTCCCTGAACTTGCAGTAAGGTC |
| | | | AGCTTCAGGACCTGTTCCAGTGGGCACTGTACTTGGATC |
| | | | TTCCCGGCGTGTGTGTGCCTTACACAGGGGTGAACTGTT |
| | | | CACTGTGGTGATGCATGATGAGGGTAAATGGTAGTTGA |
| | | | AAGGAGCAGGGGCCCTGGTGTTGCATTTAGCCCTGGGG |
| | | | CATGGAGCTGAACAGTACTTGTGCAGGATTGTTGTGGC |
| | | | TACTAGAGAACAAGAGGGAAAGTAGGGCAGAAACTGG |
| | | | ATACAGTTCTGAGGCACAGCCAGACTTGCTCAGGGTGG |
| | | | CCCTGCCACAGGCTGCAGCTACCTAGGAACATTCCTTG |
| | | | CAGACCCCGCATTGCCCTTTGGGGGTGCCCTGGGATCC |
| | | | CTGGGGTAGTCCAGCTCTTCTTCATTTCCCAGCGTGGCC |
| | | | CTGGTTGGAAGAAGCAGCTGTCACAGCTGCTGTAGACA |
| | | | GCTGTGTTCCTACAATTGGCCCAGCACCCTGGGGCACG |
| | | | GGAGAAGGGTGGGGACCGTTGCTGTCACTACTCAGGCT |
| | | | GACTGGGGCCTGGTCAGATTACGTATGCCCTTGGTGGTT |
| | | | TAGAGATAATCCAAAATCAGGGTTTGGTTTGGGGAAGA |
| | | | AAATCCTCCCCCTTCCTCCCCCGCCCCGTTCCCTACCGC |
| | | | CTCCACTCCTGCCAGCTCATTTCCTTCAATTTCCTTTGAC |
| | | | CTATAGGCTAAAAAAGAAAGGCTCATTCCAGCCACAGG |
| | | | GCAGCCTTCCCTGGGCCTTTGCTTCTCTAGCACAATTAT |
| | | | GGGTTACTTCCTTTTTCTTAACAAAAAAGAATGTTTGAT |
| | | | TTCCTCTGGGTGACCTTATTGTCTGTAATTGAAACCCTA |
| | | | TTGAGAGGTGATGTCTGTGTTAGCCAATGACCCAGGTG |
| | | | AGCTGCTCGGGCTTCTCTTGGTATGTCTTGTTTGGAAAA |
| | | | GTGGATTTCATTCATTTCTGATTGTCCAGTTAAGTGATC |
| | | | ACCAAAGGACTGAGAATCTGGGAGGGCAAAAAAAAAA |
| | | | AAAAAGTTTTTATGTGCACTTAAATTTGGGGACAATTTT |
| | | | ATGTATCTGTGTTAAGGATATGTTTAAGAACATAATTCT |
| | | | TTTGTTGCTGTTTGTTTAAGAAGCACCTTAGTTTGTTTA |
| | | | AGAAGCACCTTATATAGTATAATATATATTTTTTTGAAA |
| | | | TTACATTGCTTGTTTATCAGACAATTGAATGTAGTAATT |
| | | | CTGTTCTGGATTTAATTTGACTGGGTTAACATGCAAAAA |
| | | | CCAAGGAAAAATATTTAGTTTTTTTTTTTTTTTTTGTATA |
| | | | CTTTTCAAGCTACCTTGTCATGTATACAGTCATTTATGC |
| | | | CTAAAGCCTGGTGATTATTCATTTAAATGAAGATCACAT |
| | | | TTCATATCAACTTTTGTATCCACAGTAGACAAAATAGCA |
| | | | CTAATCCAGATGCCTATTGTTGGATACTGAATGACAGA |
| | | | CAATCTTATGTAGCAAAGATTATGCCTGAAAAGGAAAA |
| | | | TTATTCAGGGCAGCTAATTTTGCTTTTACCAAAATATCA |
| | | | GTAGTAATATTTTTGGACAGTAGCTAATGGGTCAGTGG |
| | | | GTTCTTTTTAATGTTTATACTTAGATTTTCTTTTAAAAAA |
| | | | ATTAAAATAAAACAAAAAAAAATTTCTAGGACTAGACG |
| | | | ATGTAATACCAGCTAAAGCCAAACAATTATACAGTGGA |
| | | | AGGTTTTACATTATTCATCCAATGTGTTTCTATTCATGTT |
| | | | AAGATACTACTACATTTGAAGTGGGCAGAGAACATCAG |
| | | | ATGATTGAAATGTTCGCCCAGGGGTCTCCAGCAACTTT |
| | | | GGAAATCTCTTTGTATTTTTACTTGAAGTGCCACTAATG |
| | | | GACAGCAGATATTTTCTGGCTGATGTTGGTATTGGGTGT |
| | | | AGGAACATGATTTAAAAAAAAACTCTTGCCTCTGCTTTC |
| | | | CCCCACTCTGAGGCAAGTTAAAATGTAAAAGATGTGAT |
| | | | TTATCTGGGGGGCTCAGGTATGGTGGGGAAGTGGATTC |
| | | | AGGAATCTGGGGAATGGCAAATATATTAAGAAGAGTAT |
| | | | TGAAAGTATTTGGAGGAAAATGGTTAATTCTGGGTGTG |
| | | | CACCAGGGTTCAGTAGAGTCCACTTCTGCCCTGGAGAC |
| | | | CACAAATCAACTAGCTCCATTTACAGCCATTTCTAAAAT |
| | | | GGCAGCTTCAGTTCTAGAGAAGAAAGAACAACATCAGC |
| | | | AGTAAAGTCCATGGAATAGCTAGTGGTCTGTGTTTCTTT |
| | | | TCGCCATTGCCTAGCTTGCCGTAATGATTCTATAATGCC |
| | | | ATCATGCAGCAATTATGAGAGGCTAGGTCATCCAAAGA |
| | | | GAAGACCCTATCAATGTAGGTTGCAAAATCTAACCCCT |
| | | | AAGGAAGTGCAGTCTTTGATTTGATTTCCCTAGTAACCT |
| | | | TGCAGATATGTTTAACCAAGCCATAGCCCATGCCTTTTG |
| | | | AGGGCTGAACAA |
| 334 | ESR1 | 2931877 | AGAAGAGAATCCTGAACTTGCATCCTAAAATAT |
| 335 | ESR1 | 2931878 | GGCAACTTGTTGACTACCCACTGGTCATTCTCCTCTGGT |
| | | | CTTATTACATACATGGATGCCAGTTTAGATTGTGTTTAT |
| | | | ATAGGAAAAATTAAATGTGTGAGCCTCCTTAAGGAACA |
| | | | TCATCAATACAGATATATCAGATAGTTCTGTCCAGCAA |
| | | | AAAACGTGCTTATTTGCTACAAGTAAATTTTTATTTATT |
| | | | TTTCTCACTTCCCTCACTCCTTCAAATTTCCAGGTAAAT |
| | | | AGCTGCCCAGGAGTTGCTTCATCTCTGTCCCAAAATACC |
| | | | TAGACAATTGCGGGATAAGGAGAATGGCAGGGAGGGA |
| | | | GTAGTGGCTAAAATCACACCCTTCAAAAGAAAGTGTGT |
| | | | AGGACACACAATTGTGAGAAGTCTGAATGCCATGCACA |
| | | | TAGGGTATGACTCACTTTGAAAATTGTTTATAATCAAGG |
| | | | AAATGAAAATGAGTTAATTTCGTGCATGCATCATTTAA |
| | | | AGCCAAATGAGAAGAAACTTCTAATTTATTTTGTTACTT |
| | | | TTCGGCTAACACTGGCAGTATGTAACAGATTTATTTTGC |
| | | | AGAAACATCTAGATTGTCCGTGATCTTGATCCTGCCCTT |
| | | | ATGTGTCTTGTCTTTGAAACCCAGTGTTTCCTGGATATA |
| | | | TGGTTCAGGAGACAAGTTTCCAGAATCAAGTTAGGACC |
| | | | CAGGTCTTCTTTTTTTCCAAACCAAACATTCTTGCTAAT |
| | | | CCTAAACTACCTGAGGCAGCCTGTGGTGGCCTCAGCTC |
| | | | TAAAACCATTGTTTAAAGGCTTCTACCCATCAATGGCCC |
| | | | TTCAGCAGAGTGGTACGGTTAACGGGGTAGGGTCTGGA |
| | | | GTCAGGGGAGACCTGGGTTCAAATCCTACATCTTTACA |
| | | | CCTCTAATCCCCAGTGTCCTTGTCTATAAATTGGGAATA |
| | | | TAGCCATGTCATGGGATTCTTGTGAGGGTTAAATGAGG |
| | | | TAAAACACATACAATGCTTAGCATGTATACAATTAAGC |
| | | | ACTAAATAATTGAAACACATTAAGTACTAAATGAATGT |
| | | | CAGCAGCTTATCACTATTATCTGTATAATGATACCAAGG |
| | | | GTGTGCCGACTCATACCCTTAGGGGT |
| 336 | ESR1 | 2931879 | AGGAAGGCCTACCTCAAATAGCAACAGAGA |
| 337 | ESR1 | 2931880 | TCTGTGCTGAGGCTCTTTGAATGCTTTGAATAA |
| 338 | ESR1 | 2931881 | CCTCTTTCAGTGTTTCGGCCAGTCATTTGCCACTTCTCAT |
| | | | TCCATCTTAGTTCTCTGTAAAGAAGGTGCCAGAGACCT |
| | | | AAGGTGCCCAAGGCAATTTTGCATTTTACAATTCTAAGC |
| | | | TTTAGAATGAAGTCATCAATTTGCTACATCCGGACTACA |
| | | | GTGCAATTATTCCTTTGCCTTGCTGGAAATTGGAGTGAA |
| | | | ATCTTTCTAGCTGTCAATTTCAACTCAGTTGCAGTAGTG |
| | | | TTTTGAAGAATTAATGGCGATAAGGTTAGAAAATTTTA |
| | | | AGTCAAACGTAGGGAAAAAGTACCAGCTAGACCATCAT |
| | | | AAGCATTTGCTTTGAAAGCATGCTTCTAAAGTGTGTTTA |
| | | | ACCTCAAATAACAGTCACAAATATGGTTATTATGAATG |
| | | | TATGCACAGATTTTTATGTTTCTAATTTTAAGAAGTTCT |
| | | | AGGGAGCTCCCTGTAACGATTTAGGGAATCTCTAGATT |
| | | | CTGATATACTGCAAGTCTTTTAATGGTAGGAATCACATT |
| | | | GAATTAATTTTGTAGGCCCAGGGCCTAAATTTAGTAGG |
| | | | TGTTCAGTACCTATTGGCATCAATTCATATGTAGGTTTA |
| | | | AAATACTGTATGAAGATACAGAATCACCACCATCAAAT |
| | | | CAAATTGAAATATGTAACAGGCTAGTATAATATTAACA |
| | | | TCTGACTTTAAACAACAACAAAGAAACCAAATGAGTAA |
| | | | CTCCTCCCTTCAAACTAATAGTCAGTTTCTTCCAACTCA |
| | | | GTCTCTTTCTCCTCTCAGGAAGAATGCGTATCTAAAAAT |
| | | | TTCCCATTGCAGACTGCTGGAAACAACATTCTAAACTAT |
| | | | TTATGCTTCTGCAATAACCTTTCCAATTTGCTGGACCAG |
| | | | TGCAAGATTAAACACGAGATATCTCAAGTCTCAATGTA |
| | | | AAGGAACACCACGACAGCCTGGACTGTGGGTGAAGTTC |
| | | | ATTCTTCCCCAGCAGACTCTGCCTTTCATTCTCGGGGTT |
| | | | GGGTGTGCCCCAAACAGAGGTACCGACGGTAACGAAG |
| | | | CCCAAGAATGTTCAACCACAACCTGTCTGTGAAGGTGT |
| | | | TGGATGACGTTTGCCATTCAGGTGAAGATTATTTATGTT |
| | | | CCAGTCCCACCTGAGTAGCAAAGTGAACACTGTGCTGA |
| | | | ATGCTCAGAAAGATGTTAATGAACCGTGCTGGACAGAG |
| | | | CAGAGCTGAAAGGCGCCTTGCGAGTGTCGTAGTGAGAA |
| | | | TGTGGCTGTCCCAGCTGCAAAGCCCTGTTAGGAGGCAT |
| | | | GAGGAAGCACTTGCTGCCCTAAGAAACGATGCCTTCGA |
| | | | CATTTTCAAAAGATCTATGTGGCTGTCTGAAACAATGC |
| | | | GGAGAGCAGATAGACGCAATATTTGGGAACCAAAGAG |
| | | | TGACTGCTGTTGGCGTTGCATCATAACATAAGCGCTTTC |
| | | | CCCCTTCTCGTCACTATCATTTGTATCAACCAAAGAACT |
| | | | GATCTCTGGTATCCTCGAAGGAATGCTGTGGGGATATT |
| | | | CTTCATCTCTGTTCATGGTACATCAGCAATTTGTGGGGA |
| | | | AAAGATGGACTATATAACACAATGATCTGCCTAAAAGA |
| | | | AACTGTCTCTACTTATAGGGGGCTGAGCAAACCTTAGA |
| | | | GCATCTGCGGATGCTCGTCATTATCTTC |
| 339 | ANLN | 2997377 | GAAATTCAAATTTGAACGGCTGCAGAGGCCGAGTCCGT |
| | | | CACTGGAAGCCGAGAGGAGAGGACAGCTGGTTGTGGG |
| | | | AGAGTTCCCCCGCCTCAGA |
| 340 | ANLN | 2997378 | ACACACTGAGCTGAGACTCACTTTTCTCTTCCTGAATTT |
| | | | GAACCACCGTTTCCATCGTCTCGTAGTCCGACGCCTGGG |
| 341 | ANLN | 2997379 | GGAGGAAGGCTTTGAGTCTGTCCTAAAAGGCTGTTGCG |
| | | | AGAGGTCTTTCAGC |
| 342 | ANLN | 2997380 | TCCTGGCGCAGCAAGAGTGAGGCGCAGGCCTGCGGAAC |
| | | | GGGTCCTGCTGGAAGCAGCTGGAATGCCCTGCAGGGCG |
| | | | GGGTCCGGGGCCGGTGACTCAGTGCGGCTGCCGCCGGG |
| | | | AAAGGCAGTAGGATGTGTGATTTGCGGAGTTCACGCAG |
| | | | CCCGCAGGGGAGATGCTAATGAAA |
| 343 | ANLN | 2997381 | AACTGCTGGAGCGAACCCGTGCCAGGCGAGAGAATCTT |
| | | | CAGAGAA |
| 344 | ANLN | 2997382 | CAGCAGCTCCAAGGTCTATGACTCATGCTAAGCGAGCT |
| | | | AGACAGCCACTTTC |
| 345 | ANLN | 2997384 | AGAAATCTTGTACAAAACCATCGCCATCAAAAAAACGC |
| | | | TGTTCTGACAACACTGAAGTAGAAGTTTCTAACTTGGA |
| | | | AAATAAACAACCAGTTGAGTCGACATCTGCAAAATCTT |
| | | | GTTCTCCAAGTCCTGTGTCTCCT |
| 346 | ANLN | 2997385 | GCCACAAGCAGCAGATACCATCAGTGATTCTGTTGCTG |
| | | | TCCCGGCATCACTGCTGGGCATGAGGAGAGGGCTGAAC |
| | | | TCAAGATTGGAAGCAACTGCAGCCTCCTCAGTTAAAAC |
| | | | ACGTATGCAAAAACTTGCAGAGCAACGGCGCCGTTGGG |
| | | | ATAATGATGATATGA |
| 347 | ANLN | 2997387 | GCTTTCAAATGCCTCGGCAACTCCAGTTGGCAGAAGGG |
| | | | GCCGTCTGGCCAATCTTGCTGCAACTATTTGCTCCTGGG |
| | | | AA |
| 348 | ANLN | 2997388 | GCCTGGTACCGCTTGTTTATCCAAATTTTCCTCTGCAAG |
| | | | TGGAGCATCTGCTAGGATCAATAGCAGCAGTGTTAAGC |
| | | | AGGAAGCTACATTCTGTTCCCAAAGGGATGGCGATGCC |
| | | | TCTTTGAATAAAGCCCTATCCTCAAGTGCTGATGATG |
| 349 | ANLN | 2997389 | TCCAGTGAAATCTACTACATCTATCACTGATGCTAAAA |
| | | | GTTGTGAGGGACAAAATCCTGAGCTACTTCCAAAAACT |
| | | | CCTATTAGTCCTCTGAAAACGGGGGTATCGAAACCAAT |
| | | | TGTGAAGTCAACTTTATCCCAGACAGTTCCATCCAAGG |
| 350 | ANLN | 2997391 | AGCCTTTCCTGGAACGCTTTGGAGAGCGTTGTCAAGAA |
| | | | CATAGCAAAGAAAGTCCAGCTCGTAGCACACCCCACAG |
| | | | AACCCCCATTATTACTCCAAATACAAAGGCCATCCAAG |
| | | | A |
| 351 | ANLN | 2997393 | AGAACTAGCATGTCTTCGTGGCCGATTTGACAAGGGCA |
| | | | ATATATGGAGTGCAGAAAAAGGCGGAAACTCAAA |
| 352 | ANLN | 2997394 | GTTTCAAAAACTCAGTCACTTCCAGTAACAGAAAAGGT |
| | | | GACCGAAAACCAGATACCAGCCAAAAATTCTAGTACAG |
| 353 | ANLN | 2997395 | CATCAGACCCAAAGGTTGAGCAGAA |
| 354 | ANLN | 2997396 | TCTTCAGTGATGTCCTAGAGGAAGGTGAACTAGATATG |
| | | | GAGAAGAGCCAAGAGGAGATGGATCAAGCATTAGCAG |
| | | | AAAGCAGCGAAGAACAGGAAGATGCACTGAATATCTC |
| | | | CTCAATGTCTTTACTTGCACCATTGGCACAAACAGTTGG |
| 355 | ANLN | 2997397 | AGTTTAGTGTCCACACCTAGACTGGAATTGAAAGACAC |
| | | | CAGCAG |
| 356 | ANLN | 2997399 | TTCAAAGAAACAGAACGTCCATCAATAAAGCAGGTGAT |
| | | | TGTTCGGAAGGAAGATGTTAC |
| 357 | ANLN | 2997400 | GAACTCAATAACGAAATAAATATGCAACAGACAGTGAT |
| | | | CTATCAAGCTAGCCAGGCTCTTAACTGCTGTGTTGATGA |
| | | | AGAACATGGAAAAGGGTCCCTAGAAGAAGCTGAAGCA |
| | | | GAAAGACTTCTTCTAATTGCA |
| 358 | ANLN | 2997402 | TGCCATCCAAAGGATCAGTTACTTTGTCAGAAATCCGCT |
| | | | TGCCTCTAAAAGCAGATTTTGTCTGCAGTACGGTTCA |
| 359 | ANLN | 2997403 | ATGGTAGCCACACCATTAGCAAGTACTTCAAACTCTCTT |
| | | | AACGGTGATGCTCTGACATTCACTACTACATTTACTC |
| 360 | ANLN | 2997406 | AAGAAAGATCCCTCAGGCCTTGATAAGAAG |
| 361 | ANLN | 2997408 | GGCCAGTCCAGGAGGTCTTAGTGCTGTGCGAACCAGCA |
| | | | ACTTCGCCCTTGTTGGATCTTACACATTATCATTGTC |
| 362 | ANLN | 2997409 | TAAGAGAGCGAGAGCTACTGGGCTATTTGTTCCAGGAA |
| | | | AA |
| 363 | ANLN | 2997414 | GTTTTGGTGCCTGGCATCGAAGATGGTGTGTTCTTTCTG |
| | | | GAAACTGTATATCTTATTGGACTTATCCAGATGATG |
| 364 | ANLN | 2997417 | TTGGCTGTTGGCTCATGTGTGCCTATATGTGTTTCTTTTC |
| | | | CCATTTTCAGGACCATTTGGTCTCGTGAATGTTTTCCTC |
| | | | CACTTTGACTCGTATCATAGGAATTCATGGCTGCCAACA |
| | | | ATCCAGGGCAGTTGTCTGCCCTTATCTTTCATAGATATA |
| | | | TAAAGAAATATTTACACATGAAATCCAATGTCTAGGTT |
| | | | TCCTTTTATAGAAAGGGGAGAAGTGGGTAAGTTGTAGA |
| | | | TAAAAGGCACTTGAGTGTGTTTCTCATTGTTATAGCTGG |
| | | | TTTTGGTACCTGGGGCTCATTATACTGTTGTTTGTATTTT |
| | | | TTATTTGAAGTTCACCATAATAAAGAGCTTTATAGGATA |
| | | | GTTGGCAAGAGCTACCAGTTGATATTT |
| 365 | ANLN | 2997418 | CATAGGAAGGATAAATCTGGCTAATTGTACCAG |
| 366 | ANLN | 2997419 | GTGCAAGACGCAACACTTTTGAATTAATTACTGTCCGA |
| | | | CCACAAAGAGAAGATGACCGAGAGACTCTTGTCAGCCA |
| | | | ATGCAGGGACACACTCTGTG |
| 367 | ANLN | 2997422 | TGGCTGTCTGCAGATACTAAAGAAGAGCGGGATCTCTG |
| | | | GATGCAAAAACTCAATCAAGTTCTTGTTGATATTCGCCT |
| | | | CTGG |
| 368 | ANLN | 2997423 | TTGCTACAAACCTATTGGAAAGCCTTA |
| 369 | ANLN | 2997424 | TACGAAAGGGTTTGTGCCAATATTCACTACGTATTATGC |
| | | | AGTATTTATATCTTTTGTATGTAAAACTTTAACTGATTT |
| | | | CTGTCATTCATCAATGAGTAGAAGTAAATACATTATAG |
| | | | TTGATTTTGCTAAATCTTAATTTAAAAGCCTCATTTTCCT |
| | | | AGAAATCTAATTATTCAGTTATTCATGACAATATTTTTT |
| | | | TAAAAGTAAGAAATTCTGAGTTGTCTTCTTGGAGCTGTA |
| | | | GGTCTTGAAGCAGCAACGTCTTTCAGGGGTTGGAGACA |
| | | | GAAACCCATTCTCCAATCTCAGTAGTTTTTTCGAAAGGC |
| | | | TGTGATCATTTATTGATCGTGATATGACTTGTTACTAGG |
| | | | GTACTGAAAAAAATGTCTAAGGCCTTTACAGAAACATT |
| | | | TTTAGTAATGAGGATGAGAACTTTTTCAAATAGCAAAT |
| | | | ATATATTGGCTTAAAGCATGAGGCTGTCTTCAGAAAAG |
| | | | TGATGTGGACATAGGAGGCAATGTGTGAGACTTGGGGG |
| | | | TTCAATATTTTATATAGAAGAGTTAATAAGCACATGGTT |
| | | | TACATTTACTCAGCTACTATATATGCAGTGTGGTGCACA |
| | | | TTTTCACAGAATTCTGGCTTCATTAAGATCATTATTTTT |
| | | | GCTGCGTAGCTTACAGACTTAGCATA |
| 370 | BLVRA | 2999566 | CCGAGAGGAAGTTTGGCGTGGTGGTGGTTGGTGTTGGC |
| | | | CGAGCCGGCTCCGTGCGGATGAGGGACTTGCGGAATCC |
| | | | ACACCCTTCCTCAGCGTTCCTGAACCTGATTGGCTTCGT |
| | | | GTCGA |
| 371 | BLVRA | 2999568 | AGCTCGGGAGCATTGATGGAGTCCAGCAGATTTCTTTG |
| | | | GAGGATGCTCTTTCCAGCCAAGAGGTGGAGGTCGCCTA |
| | | | TATCTGCAGTGAGAGCTCCAGCCA |
| 372 | BLVRA | 2999571 | TTCCTTAATGCTGGCAAGCACGTCCTTGTGGAATACCCC |
| | | | ATGACACTGTCATTGGCGGCCGCTCAGGAACTGTG |
| 373 | BLVRA | 2999573 | GAAAAGTCTTGCACGAGGAGCATGTTGAACTCTTGATG |
| | | | GAGGAATTCGCTTTCCTGAAAAAAGAAGTGGTGGGGAA |
| | | | AGACCTGCTGAAAGGGTCGCTCCTCTTC |
| 374 | BLVRA | 2999576 | AGAAGAGCGGTTTGGCTTCCCTGCATTCAGCGGCATCT |
| | | | CTCGCCTGACCTGGCTGGTCTCCCTCTTTGGGGAGCTTT |
| | | | CTCTTGTGTCTGCCACTTTGGAAGAGCGAAAGGAAGAT |
| | | | CAGTATATG |
| 375 | BLVRA | 2999577 | CCTGGTCTAAAACGAAACAGATATTTAAGCTTCCATTTC |
| | | | AAGTCTGGGTCCTTGGAGAATGTGCCAAATGTAGGAGT |
| | | | GAATAAGAACATATTTCTGAAAGATCAAAATATATTTG |
| | | | TCCAGAAAC |
| 376 | BLVRA | 2999578 | GGTTCTTCTCAAGAGTTGACCATTATCTCTATTCTTAAA |
| | | | ATT |
| 377 | EGFR | 3002642 | GGCTGCGCTCTGCCCGGCGAGTCGGGCT |
| 378 | EGFR | 3002666 | ATGATCCTTTGCCTGGACTTTCTAAGTGCCC |
| 379 | EGFR | 3002667 | GTTGCACTTGGCCTAGCATTCCAACCTCACCTGCCTCAG |
| | | | CTTGTTCAACCTGAAAACCTACCAAGTGAAAGCAAGAG |
| | | | CCACGTGAAGACGCCTTAGTTATATGCACCCACCCAGA |
| | | | CACTTG |
| 380 | EGFR | 3002717 | AACTACAGGCCTTTTGAGAGAGTGCCCTCCTAATGAAT |
| | | | TGAGTACCTATTTCTCCATACACAGTGTCTATCATGACC |
| | | | TACAAACCCTTTTCCCATGAGGTGTAACAGAGAGAGAT |
| | | | TACAGCCTTGGAACTGGATGTCAGACTCTCCTGGTTTAA |
| | | | GACAATAAGCCATGACATAGAGCCTGAAACCA |
| 381 | EGFR | 3002729 | CAAGCTCACGCAGTTGGGCACTTTTGAAGATCATTTTCT |
| | | | CAGCCTCCAGAGGATGTTCAATAACTGTGAGGTGGTCC |
| | | | TTGGGAATTTGGAAATTACCTATGTGCAGAGGAATTAT |
| | | | GATCTTTCC |
| 382 | EGFR | 3002731 | AGGTGGCTGGTTATGTCCTCATTGCCCTCAACACAGTGG |
| | | | AGCGAATTCCTTTGGAAAACCTGCAGATCATCAGAGGA |
| | | | AATATGTACTACGAAAATTCCTATGCCTTAGCAGTCTTA |
| | | | TCTA |
| 383 | EGFR | 3002733 | GGCGCCGTGCGGTTCAGCAACAACCCTGCCCTGTGCAA |
| | | | CGTGGAGAGCATCCAGTGGCGGGACATAGTCAGCAGTG |
| | | | ACTTTCTCAGCAACATGTCGATGGA |
| 384 | EGFR | 3002738 | GCCAAAAGTGTGATCCAAGCTGTCCCAATGGGAGCTGC |
| | | | TGG |
| 385 | EGFR | 3002741 | AAAATCATCTGTGCCCAGCAGTGCTCCGGGCGCTGCCG |
| | | | TGGCAAGTCCCCCAGTGACTGCTGCCACAACCAGTGTG |
| | | | CTGCAGGCTGCACAGGCCCCCGGGAGAGCGACT |
| 386 | EGFR | 3002743 | CGAAGCCACGTGCAAGGACACCTGCCCCCCACTCATGC |
| | | | TCTACAACCCCACCACGTACCAGATGGATGTGAACCCC |
| | | | GAGGGCAAATACAGCTTTGGTGCCACCTGCGTGAAGA |
| 387 | EGFR | 3002747 | TAATTATGTGGTGACAGATCACGGCTCGTGCGTCCGAG |
| | | | CCTGTGGGGCCGACAGCTATGAGATGGAGGAAGACGG |
| | | | CGTCCGCAAGTGTAAGAAGTG |
| 388 | EGFR | 3002750 | TGGATATTCTGAAAACCGTAAAGGA |
| 389 | EGFR | 3002751 | TGTTTTAGAGAGAGAACTTTTCGACATATTTCCTGTTCC |
| | | | CTTGGAATA |
| 390 | EGFR | 3002757 | TTTTCTCTTGCAGTCGTCAGCCTGAACATAACATCCTTG |
| | | | GGATTACGCTCCCTCAAGGAGATAAGTGATGGAGATGT |
| | | | GATAATTTCAGGAAACAAAAATTTGTGCTATGC |
| 391 | EGFR | 3002760 | CACAGGCCAGGTCTGCCATGCCTTGTGCTCCCCCGAGG |
| | | | GCTGCTGGGGCCCGGAGCCCAGGGACTGCGTCTCTTGC |
| | | | CGGAATGTCAGCCGAGGCAGGGAATGCGTGGACAAGT |
| | | | GCAACCTTCTGGAG |
| 392 | EGFR | 3002763 | TGTATCCAGTGTGCCCACTACATTGACGGCCCCCACTGC |
| | | | GTCAAGACCTGCCCGGCAGGAGTCATGGGAGAAAACA |
| | | | ACACCCTGGTCTGGAAGTACGCAGACGCCGGCCATGTG |
| | | | TGCCACCTG |
| 393 | EGFR | 3002769 | GCCAGGAAATGAGAGTCTCAAAGCCATGTTATTCTGCC |
| | | | TTTTTAAACTATCATCCTGTAATCAAAGTAATGATGGCA |
| | | | GCGTGTCCCACCAGAGCGGGAGCCCAGCTGCTCAGGAG |
| | | | TCATGCTTAGGATGGATCCCTTCTCTTCTGCCGTCAGAG |
| | | | TTTCAGCTG |
| 394 | EGFR | 3002770 | GCCTCATGCCTTCACGTGTCTGTTCCCCCCGCTTTTCCTT |
| | | | TCTGCCACCCCTGCACGTGGGCCGCCAGGTTCCCAAGA |
| | | | GTATCCTACCCATTTCCTTCCTTCCACTCCCTTTGCCAGT |
| | | | GCCTCTCACCCCAACTAGTAGCTAACCATCACCCCCAG |
| | | | GACTGACCTCTTCCTCCTCGCTGCCAGATGATTGTTCAA |
| | | | AGCACAGAATTTGTCAGAAACCTGCAGGGACTCCATGC |
| | | | TGCCAGCCTTCTCCGTAATTAGCATGGCCCCAGTCCATG |
| | | | CTTCTAGCCTTGGTTCCTTCTGCCCCTCTGTTTGAAATTC |
| | | | TAGAGCCAGCTGTG |
| 395 | EGFR | 3002771 | GCCAGGTCTTGAAGGCTGTCCAACG |
| 396 | EGFR | 3002774 | AAGCTACATAGTGTCTCACTTTCCA |
| 397 | EGFR | 3002775 | GCCTAAGATCCCGTCCATCGCCACTGGG |
| 398 | EGFR | 3002776 | GTGGCCCTGGGGATCGGCCTCTTCATGCGAAGGCGCCA |
| | | | CATCGTTCGGAAGCGCACGCTG |
| 399 | EGFR | 3002778 | GCCTCTTACACCCAGTGGAGAAGCTCCCAACCAAGCTC |
| | | | TCTTGAGGATCTTGAAGGAAACTGAATTCAAAAAGATC |
| | | | AAAGTGCTGGGCTCCGGTGCGTTCGGCACGGTGTAT |
| 400 | EGFR | 3002779 | TTAAAATTCCCGTCGCTATCAAGGAATTAAGAGAAGCA |
| | | | ACATCTCCGAAAGCCAACAAG |
| 401 | EGFR | 3002786 | CTACGTGATGGCCAGCGTGGACAACCCCCACGTGTGCC |
| | | | GCCTGCTGGGCATCTGCCTCACCTCCACCGTGCAGCTCA |
| | | | TCACGCAGCTCATGCCCTTCGGCTGCCTCCTGGACTATG |
| | | | TCCGGGAACACAAAGACAATATTGGCTCCCAGTACCTG |
| | | | CTCAACTG |
| 402 | EGFR | 3002798 | GGCATGAACTACTTGGAGGACCGTCGCTTGGTGCACCG |
| | | | CGACCTGGCAGCCAGGAACGTACTGGTGAAAACACCGC |
| | | | AGCATGTCAAGATCACAGATTTTGGGCTGGCCAAACTG |
| | | | CTGGGTGCGGAAGAGAAAGAATACC |
| 403 | EGFR | 3002800 | GTGCCTATCAAGTGGATGGCATTGGAATCAATTTTACA |
| | | | CAGAATCTATACCCACCAGAGTGA |
| 404 | EGFR | 3002801 | AGTTGATGACCTTTGGATCCAAGCCATATGACGGAATC |
| | | | CCTGCCAGCGAGATCTCCTCCATCCTGGAGAAAGGAGA |
| | | | ACGCCTCCCTCAGCCACCCATATGTACCATCGATGTCTA |
| | | | CA |
| 405 | EGFR | 3002802 | CTGGATGATAGACGCAGATAGTCGCCCAAAGTTCCGTG |
| | | | AGTTGATCATCGAATTCTCCAAAATGGCCCGAGA |
| 406 | EGFR | 3002803 | GCTTCCATTGGGAAGAGTCCCTCTAATGAGCATCTCATG |
| | | | TCACTGTGTTCTGTCACATGCCAGCCTGGCCTCCCTGTG |
| | | | TCCCAGATCGCATTATTAAACCCTCCAGCGCATTAGAG |
| | | | CAAGCCTCAGTAAGGCGCAGGCCACATCGTGAACTAAG |
| | | | CAGCATCCGTGAGTGGGGCCCACCCAACTCCATCTCCC |
| | | | CCTCCCCGTCTGAACTCTCCTCTGGTGCTCGTCCTCACT |
| | | | GTCCGGCTAG |
| 407 | EGFR | 3002806 | GGGGATGAAAGAATGCATTTGCCAAGTCCTACAGACTC |
| | | | CAACTTCTACCGTGCCCTGATGGATGAAGAAGACATGG |
| | | | ACGACGTGGTGGATGCCGACGAGTACCTCATCC |
| 408 | EGFR | 3002808 | GACAGCTTCTTGCAGCGATACAGCTCAGACCCCACAGG |
| | | | CGCCTTGACTGAGGACAGCATAGACGACACCTTCCTC |
| 409 | EGFR | 3002809 | TCCTGCTCCTCAACCTCCTCGACCCACTCAGCAG |
| 410 | EGFR | 3002810 | CCTCCAGCATCTCCAGAGGGGGAAACAGTG |
| 411 | EGFR | 3002811 | GTCAACAGCACATTCGACAGCCCTGCCCACTGGGCCCA |
| | | | GAAAGGCAGCCACCAAATTAGCCTGGACAACCCTGACT |
| | | | ACCAGCAGGACTTCTTTCCCAAGGAAGCCAAGCCAAAT |
| | | | GGCATCTTTAAGGGCTCCACAGCTGAAAATGCAGAATA |
| | | | CCTAAGGGTCGCGCCACAAAGCAGTGAAT |
| 412 | EGFR | 3002812 | CCACAGACTGGTTTTGCAACGTTTACACCGACTAGCCA |
| | | | GGAAGTACTTCCACCTCGGGCACATTTTGGGAAGTTGC |
| | | | ATTCCTTTGTCTTCAAACTGTGAAGCATTTACAGAAACG |
| | | | CATCCAGCAAGAATATTGTCCCTTTGAGCAGAAATTTAT |
| | | | CTTTCAAAGAGGTATATTTGAAAAAAAAAAAAAGTATA |
| | | | TGTGAGGATTTTTATTGATTGGGGATCTTGGAGTTTTTC |
| | | | ATTGTCGCTATTGATTTTTACTTCAATGGGCTCTTCCAA |
| | | | CAAGGAAGAAGCTTGCTGGTAGCACTTGCTACCCTGAG |
| | | | TTCATCCAGGCCCAACTGTGAGCAAGGAGCACAAGCCA |
| | | | CAAGTCTTCCAGAGGATGCTTGATTCCAGTGGTTCTGCT |
| | | | TCAAGGCTTCCACTGCAAAACACTAAAGATCCAAGAAG |
| | | | GCCTTCATGGCCCCAGCAGGCCGGATCGGTACTGTATC |
| | | | AAGTCATGGCAGGTACAGTAGGATAAGCCACTCTGTCC |
| | | | CTTCCTGGGCAAAGAAGAAACGGAGGGGATGGAATTCT |
| | | | TCCTTAGACTTACTTTTGTAAAAATGTCCCCACGGTACT |
| | | | TACTCCCCACTGATGGACCAGTGGTTTCCAGTCATGAGC |
| | | | GTTAGACTGACTTGTTTGTCTTCCATTCCATTGTTTTGAA |
| | | | ACTCAGTATGCTGCCCCTGTCTTGCTGTCATGAAATCAG |
| | | | CAAGAGAGGATGACACATCAAATAATAACTCGGATTCC |
| | | | AGCCCACATTGGATTCATCAGCATTTGGACCAATAGCC |
| | | | CACAGCTGAGAATGTGGAATACCTAAGGATAGCACCGC |
| | | | TTTTGTTCTCGCAAAAACGTATCTCCTAATTTGAGGCTC |
| | | | AGATGAAATGCATCAGGTCCTTTGGGGCATAGATCAGA |
| | | | AGACTACAAAAATGAAGCTGCTCTGAAATCTCCTTTAG |
| | | | CCATCACCCCAACCCCCCAAAATTAGTTTGTGTTACTTA |
| | | | TGGAAGATAGTTTTCTCCTTTTACTTCACTTCAAAAGCT |
| | | | TTTTACTCAAAGAGTATATGTTCCCTCCAGGTCAGCTGC |
| | | | CCCCAAACCCCCTCCTTACGCTTTGTCACACAAAAAGTG |
| | | | TCTCTGCCTTGAGTCATCTATTCAAGCACTTACAGCTCT |
| | | | GGCCACAACAGGGCATTTTACAGGTGCGAATGACAGTA |
| | | | GCATTATGAGTAGTGTGGAATTCAGGTAGTAAATATGA |
| | | | AACTAGGGTTTGAAATTGATAATGCTTTCACAACATTTG |
| | | | CAGATGTTTTAGAAGGAAAAAAGTTCCTTCCTAAAATA |
| | | | ATTTCTCTACAATTGGAAGATTGGAAGATTCAGCTAGTT |
| | | | AGGAGCCCACCTTT |
| 413 | EGFR | 3002813 | GGCACCCTGACCGAGGAAACAGCTGCCAGAGGCCTCCA |
| | | | CTGCTAAAGTCCACATAAGGCTGAGGTCAGTCACCCTA |
| | | | AACAACCTGCTCCCTCTAAGCCAGGGGATGAGCTTGGA |
| | | | GCATCCCACAAGTTC |
| 414 | EGFR | 3002814 | GAAATATTTCAGTCAGAACTGGGAAACAGAAGGACCTA |
| | | | CATTCTGCTGTCACTTATGTGTCAAGAAGCAGATGATCG |
| | | | ATGAGGCA |
| 415 | EGFR | 3002815 | GTCAGTTGTAAGTGAGTCACATTGTAGCATTAAATTCTA |
| | | | GTATTTTTGTAGTTTGAAACAGTAACTTAATA |
| 416 | EGFR | 3002816 | CACAGTTCTGTCTGGTAGAAGCCGCAAAGCCCTTAGCC |
| | | | TCTTCACGGATCT |
| 417 | EGFR | 3002817 | GATAGCCTGGCCTTAATACCCTACAGAAAGCCTGTCCA |
| | | | TTGGCTGTTTCTTCCTCAGTCAGTTCCTGGAAGACCTTA |
| | | | CCCCATGACCCCAGCTTCAGATGTGGTCTTTGGAAACA |
| | | | GAGGTCGAAGGAA |
| 418 | EGFR | 3002818 | CTAGGCCTCTGATTGCACTTGTGTAGGATGAAGCTGGT |
| | | | GGGTGATGGGAACTCAGCACCTCCCCTCAGGCAGAAAA |
| | | | GAATCATCTGTGGAGCTTCAAAAGAAGGGGCCTGGAGT |
| | | | CTCTGCAGACCAATTCAACCCAAATCTCGGGGGCTCTTT |
| | | | CATGATTCTAATGGGCAACCAGGGTTGAAACCCTTATTT |
| | | | CTAGGGTCTTCAGTTGTACAAGACTGTGGGTCTGTACCA |
| | | | GAGCCCCCGTCAGAGTAGAATAAAAGGCTGGGTAGGGT |
| | | | AGAGATTCCCATGTGCAGTGGAGAGAACAATCTGCAGT |
| | | | CACTGATAAGCCTGAGACTTGGCTCATTTCAAAAGCGT |
| | | | TCAATTCATCCTCACCAGCAGTTCAGCTGGAAAGGGGC |
| | | | AAATACCCCCACCTGAGCTTTGAAAACGCCCTGGGACC |
| | | | CTCTGCATTCTCTAAGTAAGTTATAGAAACCAGTCTCTT |
| | | | CCCTCCTTTGTGAGTGAGCTGCTATTCCACGTAGGCAAC |
| | | | ACCTGTTGAAATTGCCCTCAATGTCTACTCTGCATTTCT |
| | | | TTCTTGTGATAAGCACACACTTTTATTGCAACATAATGA |
| | | | TCTGCTCACATTTCCTTGCCTGGGGGCTGTAAAACCTTA |
| | | | CAGAACAGAAATCCTTGCCTCTTTCACCAGCCACACCT |
| | | | GCCATACCAGGGGTACAGCTTTGTACTATTGAAGACAC |
| | | | AGACAGGATTTTTAAATGTAAATCTATTTTTGTAACTTT |
| | | | GTTGCGGGATATAGTTCTCTTTATGTAGCACTGAACTTT |
| | | | GTACAATATATTTTTAGAAACTCATTTTTCTACTAAAAC |
| | | | AAACACAGTTTACTTTAGAGAGACTGCAATAGAATCAA |
| | | | AATTTGAAACTGAAATCTTTGTTTAAAAGGGTTAAGTTG |
| | | | AGGCAAGAGGAAAGCCCTTTCTCTCTCTTATAAAAAGG |
| | | | CACAACCTCATTGGGGAGCTAAGCTAGGTCATTGTCAT |
| | | | GGTGAAGAAGAGAAGCATCGTTTTTATATTTAGGAAAT |
| | | | TTTAAAAGATGATGGAAAGCACATTTAGCTTGGTCTGA |
| | | | GGCAGGTTCTGTTGGGGCAGTGTTAATGGAAAGGGCTC |
| | | | ACTGTTGTTACTACTAGAAAAATCCAGTTGCATGCCATA |
| | | | CTCTCATCATCTGCCAGTGTAACCCTGTACATGTAAGAA |
| | | | AAGCAATAACATAGCACTTTGTTGGTTTATATATATAAT |
| | | | GTGACTTCAATGCAAATTTTATTTTTATATTTACAATTG |
| | | | ATATGCATTTACCAGTATAAACTAGACATGTCTGGAGA |
| | | | GCCTAATAATGTTCAGCACACTTTGGTTAGTTCACCAAC |
| | | | AGTCTTACCAAGCCTGGGCCCAGCCACCCTAGAGAAGT |
| | | | TATTCAGCCCTGGCTGCAGTGACATCACCTGAGGAGCT |
| | | | TTTAAAAGCTTGAAGCCCAGCTACACCTCAGACCGATT |
| | | | AAACGCAAATCTCTGGGGCTGAAACCCAAGCATTCGTA |
| | | | GTTTTTAAAGCTCCTGAGGTCATTCCAATGTGCGGCCAA |
| | | | AGTTGAGAACTACTGGCCTAGGGATTAGCCACAAGGAC |
| | | | ATGGACTTGGAGGCAAATTCTGCAGGTGTATGTGATTC |
| | | | TCAGGCCTAGAGAGCTAAGACACAAAGACCTCCACATC |
| | | | TGTCGCTGAGAGTCAAGAACCTGAACAGAGTTTCCATG |
| | | | AAGGTTCTCCAAGCACTAGAAGGGAGAGTGTCTAAACA |
| | | | ATGGTTGAAAAGCAAAGGAAATATAAAACAGACACCT |
| | | | CTTTCCATTTCCTAAGGTTTCTCTCTTTATTAAGGGTGG |
| | | | ACTAGTAATAAAATATAATATTCTTGCTGCTTATGCAGC |
| | | | TGACATTGTTGCCCTCCCTAAAG |
| 419 | EGFR | 3002819 | GAAAACGCTGGCCTATCAGTTACATTACAAAA |
| 420 | EGFR | 3002820 | ACTACCTGCAGTGTGTCCTCTGAGGCTGCAAGTCTGTCC |
| | | | TATCTGAATTCCCAGCAGAAGCACTAAGAAGCTCCACC |
| | | | CTATCACCTAGCAGATAAAACTATGGGGAAAACTTAAA |
| | | | TCTGTGCATACATTTCTGGATGCATTTACTTATCTTTAA |
| | | | AAAAAAAGGAATCCTATGACCTGATTTGGCCACAAAAA |
| | | | TAATCTTGCTGTACAATACAATCTCTTGGAAATTAAGAG |
| | | | ATCCTATGGATTTGATGACTGGTATTAGAGGTGACAAT |
| | | | GTAACCGATTAACAACAGACAGCAATAACTTCGTTTTA |
| | | | GAAACATTCAAGCAATAGCTTTATAGCTTCAACATATG |
| | | | GTACGTTTTA |
| 421 | EGFR | 3002827 | CCAGCTGCTGCTCTTATTGGGCTAAGGGATGAAGACTA |
| | | | CACAGGCTGTCGAGTTCTCCTCCAGGATACTCACTGACT |
| | | | CCCAGGTGGCCTACTCTATCAACCCATGAGCATGGTCA |
| | | | GCCCTGAGACACTGTCAGGAAGTGAGAGGCCACTGTAG |
| | | | GAGTGTCACGCTAACAGCTAACCCAGCGGCCCCCATGA |
| | | | AGGCTGACGCTTGGCATCCCTGGAGAATGGGTCACACT |
| | | | TTTACCTCATCGCATAGCCATAGTCTCTGCCTCCACAGC |
| | | | CTTCATTTTCTCACTTTTTCTATCAATGCCCAAGAAAAG |
| | | | AATGTTTTTCCATGCAAAACCCAGCAAAGAAATAAAGT |
| | | | CCCCAAGCCTGTGTGCAGGGTTAAAGGAACCTCATGAG |
| | | | AGTGAAGCACTAGGCTGCGAAGGGGAGAAACCCACTT |
| | | | GGGAAAAGCCCACCACTTCGGGGAACCTGGATGACGGC |
| | | | CCATAGAGCATAGTGCCACAAACAGTGACTCTGAAAGT |
| | | | TCGTGTTCCATGGTTCCAAGATTTGAAGTAGTGTTACTC |
| | | | CTCAGAGGCCTTTGTTACTAATCCCCACGGTGGGCTTTT |
| | | | TCCACCACAGGTTTTAACCTAGCTTTGAACTATGTTCAA |
| | | | GTCCTATGAGGTTGCAAAGCACCACCCCAATATTCTTAT |
| | | | ACCTATAACGGGGCACAGAAGCTACTGAAGCATTTGGT |
| | | | AGGGCTGTGGCTGCCTGTGCAGTGGGCTATGTGAAAAT |
| | | | CAACTGGAGTCTCCTGGAGAGTGGGCTCTGGAAGTCTC |
| | | | CTCAGTCATCAACGCAGGCTCTTGACCTCTCGTGCACTA |
| | | | TATGACCAAAGTCTACGCAGGCTATGTGGGCTATTTGC |
| | | | GATTCAAGTCCAGCTATGTGCTGCTTCTAACCCAGGCA |
| | | | GTATTCCAGACTGCAGTCCAGCTGAGCGTGGAATGTGT |
| | | | ATCCTGGTGCCCGGATTCCACACTTCACTGCCTTGGTGA |
| | | | TTCAGCCGACCCTCA |
| 422 | EGFR | 3002828 | GCATTTTGCTAAGTCCCTGAGGGTCACTGGTCCTCAAAG |
| | | | CGGCATGGCGGCATGGCGTGGCTGGTTCTGCCACATGC |
| | | | CAGCTGTGTGACCTCTGAGACTCCACTTCTTCAGTGCTG |
| | | | AAAATAAAGAAGGAGTTTTACTAAGGACCAAACAAGA |
| | | | TAATGAATGTGAAACTGCTCCACGAACCCCAAAGAATT |
| | | | ATGCACATAGATGCGATCATTAAGATGCGAAGCCATCG |
| | | | AGTTACCACCTGGCATGCTTAAACTGTAAA |
| 423 | ACTR3B | 3032458 | CTGCGGCGGCTCGCGGGAGACGCTGCGCGCGGGGCTAG |
| | | | CGGGCGGCGGAGCGGACGGCGACGGGGCGCTCTCGGG |
| | | | CT |
| 424 | ACTR3B | 3032459 | TCCCTGCCTCCCTGCGTGGTGGACTGTGGCACC |
| 425 | ACTR3B | 3032468 | AACTATCTCCTTCCTGCTGTAATCAGT |
| 426 | ACTR3B | 3032488 | AGGCTCCACCATGTTCAGGGATTTCGGACGCCGACTGC |
| | | | AGAGGGATTT |
| 427 | ACTR3B | 3032489 | GAGGTCCAGGTGGTCACGCATCACATGCAGCGCTACGC |
| | | | CGTGTGGTTCGGAGGCTCCATGCT |
| 428 | ACTR3B | 3032491 | AAGGACTATGAAGAGTACGGGCCCAGCATCTGCCGCCA |
| | | | CAACCCCGTCTTTGGAGTCATGT |
| 429 | ACTR3B | 3032492 | TTCGATGGTGTCACGTTGGGGAACAAGTGTCCTTCAGA |
| | | | ACCCAGAGAAGGCCGCCGTTCTGTAAATAGCGACGTCG |
| | | | GTGTTGCTGCCCAGCAGCGTGCTTGCATTGC |
| 430 | ACTR3B | 3032493 | CCATTTATCCGTGTGCCGACCGCTGTCTGCCAGCCTCCT |
| | | | CCTTCTCCCGCCCTCCTCACCCTCGCTCTCCCTCCTCCTC |
| | | | CTCCTCCGAGCTGCTAGCTGACAAATACAATTCTGAAG |
| | | | GAATCCAAATGTGACTTTGAAAATTGTTAGAGAAAACA |
| | | | ACATTAGAAAATGGCGCAAAATCGTTAGGTCCCAGGAG |
| | | | A |
| 431 | ACTR3B | 3032494 | GTACAACTGGCTGATACTAAGCACGAATAGATATTGAT |
| | | | GTTATGGAGTGCTGTAATCCAAAGTTTTTAATTGTGAGG |
| | | | CATGTTCTGATATGTTTATAGGCAAACAAATAAAACAG |
| | | | CAAACTTTTTTGCCACATGTTTGCTAGAAAATGATTATA |
| | | | CTTTATTGGAGTGACATGAAGTTTGAACACTAAACAGT |
| | | | AATGTATGAGAATTACTACAGATACATGTATCTTTTAGT |
| | | | TTTTTTTGTTTGAACTTTCTGGAGCTGTTTTATAGAAGAT |
| | | | GATGGTTTGTTGTCGGTGAGTGTTGGATGAAATACTTCC |
| | | | TTGCACCATTGTAATAAAAGC |
| 432 | NAT1 | 3087948 | ATTGACCCAGTCGACAGGATCTGAACTTCCAGT |
| 433 | NAT1 | 3087952 | GGACCTGTTCCAAGCTCTCACGTTCCACATCACACATGG |
| | | | GACATCTAGTGTCAGGCTCCCAGAGAGCAGGAACCAGG |
| | | | TGAAATATAAGAGCACAGTCCTCCCAGCCGGTGGCATG |
| | | | GGGATAATCGGACAATACAACTCTC |
| 434 | NAT1 | 3087956 | CCCTGAACATGGACTTGCAGAATTCCACAGAAGAGAGG |
| | | | AGACTGGCCTAGACAGACAGCCCCAGGAGCTGAGGGC |
| | | | CCAACAGGCTTTCTACCCTGGATGCTGCTCCCATGCCCT |
| | | | GACATGAGGCCCACTACA |
| 435 | NAT1 | 3087957 | CAGCCTGGATGTGAACTGCAACTCCAAAGTGTGTCCAG |
| | | | ACTCAAGGCAAGGGCACTAGGCTTTCCAGACCTCCTAC |
| | | | TAAGTCATTGATCCAGCACTGCCCTGCCAGGACATAAA |
| | | | TCCCTGGCACCTCTTGCTCTCTGCAAAGGAGGGCAAAG |
| | | | CAGCTTCAGGAGCCCTTGGGAGTCCTCCAAAGAGAGTC |
| | | | TAGGGTACAGGTC |
| 436 | NAT1 | 3087958 | ACAGAAGGGCCATGCTGTTATTACTCTTACACAAGGAG |
| | | | GCAGCCCTCGAGCCACAGGGTCCAGCTGTTGGCTATAA |
| | | | TAGCCTACCGGTCTCTGATGATCACCATGTTT |
| 437 | NAT1 | 3087960 | GCAGCAATCTGTCTTCTGGATTAAAACTGAAGATCAAC |
| | | | CTACTTTCAACTTACT |
| 438 | NAT1 | 3087961 | AAATATAGCCATAATTAGCCTACTCAAATCCAAGTGTA |
| | | | AA |
| 439 | NAT1 | 3087962 | CCATTGTGGGGATGCCATGGACTTAGGCTTAGAGGCCA |
| | | | TTTTTGATCAAGTTGTGAGAAGAAATCGGGGTGGATGG |
| | | | TGTCTCCAGGTCAATCATCTTCTGTACTGGGCTCTGACC |
| | | | ACTATTGGTTTTGAGACCACGATGTTGGGAGGGTATGTT |
| | | | TACAGCACTCCAGCCAAAAAATACAGCACTGGCATGAT |
| | | | TCACCTTCTCCTGCAGGTGACCATTGATGGCAGGAACT |
| | | | ACATTGTCGATGCTGGGTTTGGACGCTCATACCAGATGT |
| | | | GGCAGCCTCTGGAGTTAATTTCTGGGAAGGATCAGCCT |
| | | | CAGGTGCCTTGTGTCTTCCGTTTGACGGAAGAGAATGG |
| | | | ATTCTGGTATCTAGACCAAATCAGAAGGGAACAGTACA |
| | | | TTCCAAATGAAGAATTTCTTCATTCTGATCTCCTAGAAG |
| | | | ACAGCAAATACCGAAAAATCTACTCCTTTACTCTTAAG |
| | | | CCTCGAACAATTGAAGATTTTGAGTCTATGAATACATA |
| | | | CCTGCAGACATCTCCATCATCTGTGTTTACTAGTAAATC |
| | | | ATTTTGTTCCTTGCAGACCCCAGATGGGGTTCACTGTTT |
| | | | GGTGGGCTTCACCCTCACCCATAGGAGATTCAATTATA |
| | | | AGGACAATACAGATCTAATAGAGTTCAAGACTCTGAGT |
| | | | GAGGAAGAAATAGAAAAAGTGCTGAAAAATATATTTA |
| | | | ATATTTCCTTGCAGAGAAAGCTTGTGCCCAAACATGGT |
| | | | GATAGA |
| 440 | NAT1 | 3087963 | ATAAGGAGTAAAACAATCTTGTCTATTTGTCATCCAGCT |
| | | | CACCAGTTATCAACTGACGACCTATCATGTATCTTCTGT |
| | | | ACCCTTACCTTA |
| 441 | NAT1 | 3087964 | AAAGATGGCCTGTGGTTATCTTGGAAATTGGTGATTTAT |
| | | | GCTAGAAAGCTTTTA |
| 442 | NAT1 | 3087965 | CTTGTGTAGATCTGAGTTGAAATCCTGTGGACACTGGG |
| | | | CGAATTACTTTTTAGATCTGTAGCTCTGACTCCTCAGGC |
| | | | ATAAAATGGGAATAATGCTTTTACAGTTTAGTGGCGGA |
| | | | AC |
| 443 | MYC | 3115511 | TAACGCGCTCTCCAAGTATACGTGGCAATGCGTTGCTG |
| | | | GGTTATTTTAATCATTCTAGGCATCGTTTTCCTCCTTATG |
| | | | CCTCTATCATTCCTCCCTATCTACACTAACATCCCACGC |
| | | | TCTGAACGCGCGCCCATTAATACCCTTCTTTCCTCCACT |
| | | | CTCCCTGGGACTCTTGATCAAAGCGC |
| 444 | MYC | 3115512 | ATGCGGTTTGTCAAACAGTACTGCTACGGAGGAGCAGC |
| | | | AGAGA |
| 445 | MYC | 3115513 | GTAGGCGCGCGTAGTTAATTCATGCGGCTCTCTTACTCT |
| | | | GTTTACATCCTAGAGCTAGAGTGCTCGGCTGCCCGGCT |
| | | | GAGTCTCCTCCCCACCTTCCCCACCCTCCCCACCCTCCC |
| | | | CATAAGCGCCCCTCCCGGGTTCCCAAAGCAGAGGGCGT |
| | | | GGGGGAAAAGAAAAAAGATCCTCTCTCGCTAATCTCCG |
| | | | CCCACCGGCCCTTTATAATGCGAGGGTCTGGACGGCTG |
| | | | AGGACCCCCGAGCTGTGCTGCTCGCGGCCGCCACCGCC |
| | | | GGGCCCCGGCCGTCCCTGGCTCCCCTCCTGCCTCGAGA |
| | | | AGGGCAGGGCTTCTCAGAGGCTTGGCGGGAAAAAGAA |
| | | | CGGAGGGAGGGATCGCGCTGAGTATAAAAGCCGGTTTT |
| | | | C |
| 446 | MYC | 3115514 | CAACCCTTGCCGCATCCACGAAACTTTGCCCATAG |
| 447 | MYC | 3115515 | TTACAACACCCGAGCAAGGACGCGACTCTCCCGACGCG |
| | | | GGGAGGCTATTCTGCCCATTTGGGGACACTTCCCCGCC |
| | | | GCTGCCAGGACCCGCTTCTCTGAAAGGCTCTCCTTGCAG |
| | | | CTGCTTAGACGCTGGATTTTTT |
| 448 | MYC | 3115522 | AGCCGTATTTCTACTGCGACGAGGAGGAGAACTTCTAC |
| | | | CAGCAGCAGCAGCAGAGCGAGCTGCAGCCCCCGGCGC |
| | | | CCAGCGAGGATATCTGGAAGAAATTCGAGCTGCTGCCC |
| | | | ACCCCGCCCCTGTCCCCTAGCCGCCGCTCCGGGCTCTGC |
| | | | TCGCCCTCCTACGTTGCGGTCACACCCTTCTCCCTTCGG |
| | | | GGAGACAACGACGGCGGTGGCGGGAGCTTCTCCACGGC |
| | | | CGACCAGCTGGAGATGGTGACCGAGCTGCTGGGAGGA |
| | | | GACATGGTGAACCAGAGTTTCATCTGCGACCCGGACGA |
| | | | CGAGACCTTCATCAAAAACATCATCATCCAGGACTGTA |
| | | | TGTGGAGCGGCTTCTCGGCCGCCGCCAAGCTCGTCTCA |
| | | | GAGAAGC |
| 449 | MYC | 3115523 | GAAGGACTATCCTGCTGCCAAGAGGGTCAAGTTGGACA |
| | | | GTGTCAGAGTCCTGAGACAGATCAGCAACAACCGAAAA |
| | | | TGCACCAGCCCCAGGTCCTCGGACACCGAGGAGAATGT |
| | | | CAAGAGGCGAACACACAACGTCTTGGAGCGCCAGAGG |
| | | | AGGAACGAGCTAAAACGGAGCTTTTTTGCCCTGCGTGA |
| | | | CCAGATCCCGGAGTTGGAAAACAATGAAAAGGCCCCCA |
| | | | AGGTAGTTATCCTTAAAAAAGCCACAGCATACATCCTG |
| | | | TCCGTCCAAGCAGAGGAGC |
| 450 | MYC | 3115524 | TTCCTTCTAACAGAAATGTCCTGAGCAATCACCTATGAA |
| | | | CTTGTTTCAAATGCATGATCAAATGCAACCTCACAACCT |
| | | | TGGCTGAGTCTTGAGACTGAAAGATTTAGCCATAATGT |
| | | | AAACTGCCTCAAATTGGACTTTG |
| 451 | MYC | 3115525 | TCCTAGTATATAGTACCTAGTATTATAGGTACTATAAA |
| 452 | SFRP1 | 3132783 | TGTGCAGCTCTCTAAATGGGAATTCTCAGGTAGGAAGC |
| | | | AACAGCTTCAGAAAGAGCTCAAAATAAATTGGAAATGT |
| | | | GAATCGCAGCTGTGGGTTTTACCACCGTCTGTCTCAGAG |
| | | | TCCCAGGACCTTGAGTGTCATTAGTTACTTTATTGAAGG |
| | | | TTTTAGACCCATAGCAGCTTTGTCTCTGTCACATCAGCA |
| | | | ATTTCAGAACCAAAAGGGAGGCTCTCTGTAGGCACAGA |
| | | | GCTGCACTATCACGAGCCTTTGTTTTTCTCCACAAAGTA |
| | | | TCTAACAAAACCAATGTGCAGACTGATTGGCCTGGTCA |
| | | | TTGGTCTCCGAGAGAGGAGGTTTGCCTGTGATTTCCTAA |
| | | | TTATCGCTAGGGCCAAGGTGGGATTTGTAAAGCTTTAC |
| | | | AATAATCATTCTGGATAGAGTCCTGGGAGGTCCTTGGC |
| | | | AGAACTCAGTTAAATCTTTGAAGAATATTTGTAGTTATC |
| | | | TTAGAAGATAGCATGGGAGGTGAGGATTCCAAAAACAT |
| | | | TTTATTTTTAAAATATCCTGTGTAACACTTGGCTCTTGG |
| | | | TACCTGTGGGTTAGCATCAAGTTCTCC |
| 453 | SFRP1 | 3132784 | GCGCCTGTCAGTAGTGGACATTGTAATCCAGTCGGCTT |
| | | | GTTCTTGCAGCATTCCCGCTCCCTTCCCTCCATAGCCAC |
| | | | GCTCCAAACCCCAGGGTAGCCATGGCCGGGTAAAGCAA |
| | | | GGGCCATTTAGATTAGGAAGGTTTTTAAGATCCGCAAT |
| | | | GTGGAGCAGCAGCCACTGCACAGGAGGAGGTGACAAA |
| | | | CCATTTCCAACAGCAACACAGCCACTAAAACACAAAAA |
| | | | GGGGGATTGGGCGGAAAGTGAGAGCCAGCAGCAAAAA |
| | | | CTACATTTTGCAACTTGTTGGTGTGGATCTATTGGCTGA |
| | | | TCTATGCCTTTCAACTAGAAAATTCTAATGATTGGCAAG |
| | | | TCACGTTGTTTTCAGGTCCAGAGTAGTTTCTTTCTGTCT |
| | | | GCTTTAAATGGAAACAGACTCATACCACACTTACAATT |
| | | | AAGGTCAAGCCCAGAAAGTGATAAGTGCAGGGAGGAA |
| | | | AAGTGCAAGTCCATTATGTAATAGTGACAGCAAAGGGA |
| | | | CCAGGGGAGAGGCATTGCCTTCTCTGCCCACAGTCTTTC |
| | | | CGTGTGATTGTCTTTGAATCTGAATCAGCCAGTCTCAGA |
| | | | TGCCCCAAAGTTTCGGTTCCTATGAGCCCGGGGCATGA |
| | | | TCTGATCCCCAAGACATGTGGAGGGGCAGCCTGTGCCT |
| | | | GCCTTTGTGTCAGAAAAAGGAAACCACAGTGAGCCTGA |
| | | | GAGAGACGGCGATTTTCGGGCTGAGAAGGCAGTAGTTT |
| | | | TCAAAACACATAGTTAAAAAAGAAACAAATGAAAAAA |
| | | | ATTTTAGAACAGTCCAGCAAATTGCTAGTCAGGGTGAA |
| | | | TTGTGAAATTGGGTGAAGAGCTTAGGATTCTAATCTCAT |
| | | | GTTTTTTCCTTTTCACATTTTTAAAAGAACAATGACAAA |
| | | | CACCCACTTATTTTTCAAGGTTTTAAAACAGTCTACATT |
| | | | GAGCATTTGAAAGGTGTGCTAGAACAAGGTCTCCTGAT |
| | | | CCGTCCGAGGCTGCTTCCCAGAGGAGCAGCTCTCCCCA |
| | | | GGCATTTGCCAAGGGAGGCGGATTTCCCTGGTAGTGTA |
| | | | GCTGTGTGGCTTTCCTTCCTGAAGAGTCCGTGGTTGCCC |
| | | | TAGAACCTAACACCCCCTAGCAAAACTCACAGAGCTTT |
| | | | CCGTTTTTTTCTTTCCTGTAAAGAAACATTTCCTTTGAAC |
| | | | TTGATTGCCTATGGATCAAAGAAATTCAGAACAGCCTG |
| | | | CCTGTCCCCCCGCACTTTTTACATATATTTGTTTCATTTC |
| | | | TGCAGATGGAAAGTTGACATGGGTGGGGTGTCCCCATC |
| | | | CAGCGAGAGAGTTTCAAAAGCAAAACATCTCTGCAGTT |
| | | | TTTCCCAAGTACCCTGAGATACTTCCCAAAGCCCTTATG |
| | | | TTTAATCAGCGATGTATATAAGCCAGTTCACTTAGACA |
| | | | ACTTTACCCTTCTTGTCCAATGTACAGGAAGTAGTTCTA |
| | | | AAAAAAATGCATATTAATTTCTTCCCCCAAAGCCGGAT |
| | | | TCTTAATTCTCTGCAACACTTTGAGGACATTTATGATTG |
| | | | TCCCTCTGGGCCAATGCTTATACCCAGTGAGGA |
| 454 | SFRP1 | 3132785 | AATGAAAAACCATGAGTGCCCCACCTTTCAGTCCGTGT |
| | | | TTAAGTGA |
| 455 | SFRP1 | 3132786 | CTTCCTCATCATGGGCCGCAAGGTGAAGAGCCAGTACT |
| | | | TGCTGACGGCCATCCACAA |
| 456 | SFRP1 | 3132787 | AAGTTGGGGCCCATCAAGAAGAAGGACCTGAAGAAGC |
| | | | TTGTGCTGTACCTGAAGAATGGGGCTGACTGTCCCTGCC |
| 457 | SFRP1 | 3132788 | AGAAAATGGCGACAAGAAGATTGTCCCCAAG |
| 458 | SFRP1 | 3132808 | TGTGTCCTCCCTGTGACAACGAGTTGAAATCTGAGGCC |
| | | | ATCATTGAACATCTCTGTGCCAGCGAGTTTG |
| 459 | SFRP1 | 3132810 | GCTCGGCTGCGGAAAAGGTGCTGCAGTCCACAGGGAAC |
| | | | ATGGGGGGATTGGACGGGTTGCCTGAAGAAGAGAGGA |
| | | | AAGAATCCCTCACCCCAGCCCCCAAAAGGCTGTGATGG |
| | | | GATGGGGAAACCCCATAATCGCTGTCTTCCGGACACCT |
| | | | TTTGCCCCTTGGCTGCAGTTCCACTGGTCGGCGCCCTTC |
| | | | TCAGCCTGGCTTGGAACCGTCCTCACTCA |
| 460 | SFRP1 | 3132814 | TCGGCCAGCGAGTACGACTACGTGAGCTTCCAGTCGGA |
| | | | CATCGGCCCGTACCAGAGCGGGCGCTTCTACACCAAGC |
| | | | CACCTCAGTGCGTGGACATCCCCGCGGACCTGCGGCTG |
| | | | TGCCACAACGTGGGCTACAAGAAGATGGTGCTGCCCAA |
| | | | CCTGCTGGAGCACGAGACCATGGCGGAGGTGAAGCAG |
| | | | CAGGCCAGCAGCTGGGTGCCCCTGCTCAACAAGAACTG |
| | | | CCACGCCGGCACCCAGGTCTTCCTCTGCTCGCTCTTCGC |
| | | | GCCCGTCTGCCTGGACCGGCCCATCTACCCGTGTCGCTG |
| | | | GCTCTGCGAGGCCGTGCGCGACTCGTGCGAGCCGGTCA |
| | | | TGCAGTTCTTCGGCTTCTACTGGCCCGAGATGCTTAAGT |
| | | | GTGACA |
| 461 | SFRP1 | 3132815 | GTCGCGGAGAACAGGGCGCAGAGCCGGC |
| 462 | SFRP1 | 3132816 | TCCCTGGAAGTTTGCGGCAGGACGCGC |
| 463 | SFRP1 | 3132817 | TGCAGCCTCCGGAGTCAGTGCCGCGCGCCCGCCGCCCC |
| | | | GCGCCTTCCTGCTCGCCGCACCTCCGGGAGCCGGGGCG |
| | | | CACCCAGCCCGCAGCGCCGCCTCCCCGCCCGCGCCGCC |
| | | | TCCGACCGCAGGCCGAGGGCCGCCACTGGCCGGGGGG |
| | | | ACCGGGCAGCAGCTTGCGGCCGCGGAGCCGGGCAACG |
| | | | CTGGGGACTGCGCCTTTTGTCC |
| 464 | SFRP1 | 3132819 | AATGGATCCAACTGCTTGCCCCGTCATCCCAGATGGCT |
| | | | AGGCCCCCATTCATCCCCTCTCGCTCTCCTACTGGAGGA |
| | | | ACTGCTGTATGAATCATAAAGCTCTGGGTAGGGAAGCA |
| | | | GGGAGCAGGTTCCAGGCAGAGCTGACAAGTGACTTCAC |
| | | | TTTTGAGCATCGGTTGAACCAG |
| 465 | MELK | 3168509 | AAGCGGCCACAACCCGGCGATCGAAAAGATTCTTAGGA |
| | | | ACGCCGTACCAGCCGCGTCTCTCAGGACAGCAGGCCCC |
| | | | TGTCCTTCTGTCGGGCGCCGCTCA |
| 466 | MELK | 3168510 | CCCAGTTTGCTCCTGGCTCTCGGGAGACTGGAGGATTTC |
| | | | ATCGGAGCCCCGCGCTTTACCAGCCCTGTTCCCTGGATA |
| | | | AGATATTTGACCTTTCCGACCCGCG |
| 467 | MELK | 3168511 | TTCTAATTCCAAATAAACTTGCAAGAGGACT |
| 468 | MELK | 3168512 | TGAAAGATTATGATGAACTTCTCAAATATTATGAATTAC |
| | | | ATGAA |
| 469 | MELK | 3168513 | AAAGGTCAAACTTGCCTGCCATATCCTTACTGGAGAGA |
| | | | TGGTAGCTATAAAAATC |
| 470 | MELK | 3168514 | CCCGGATCAAAACGGAGATTGAGGCCTTGAAGAACCTG |
| | | | AGACATCAGCATATATGTCAACTCTACCATGTGCTAGA |
| | | | GACAGCCAACAAAATATTCATGGTTCTTG |
| 471 | MELK | 3168515 | TACTGCCCTGGAGGAGAGCTGTTTGACTATATAATTTCC |
| | | | CAG |
| 472 | MELK | 3168516 | CAGAAGAGGAGACCCGGGTTGTCTTCCGTCAGATAGTA |
| | | | TCTGCTGTTGCTTATGTGCACAGCCAGGGC |
| 473 | MELK | 3168518 | ACAAGGATTACCATCTACAGACATGCTGTGGGAGTCTG |
| | | | GCTTATGCAGCACCTGAGTTAATACAAGGCAAATCATA |
| | | | TC |
| 474 | MELK | 3168519 | GCAGATGTTTGGAGCATGGGCATACTGTTATATGTTCTT |
| | | | A |
| 475 | MELK | 3168522 | AAGTGGCTCTCTCCCAGTAGCATTCTGCTTCTTCAA |
| 476 | MELK | 3168523 | GACCCAAAGAAACGGATTTCTATGA |
| 477 | MELK | 3168524 | GCAAGATTACAACTATCCTGTTGAGTGGCAAAGCAAGA |
| 478 | MELK | 3168525 | TTTATTCACCTCGATGATGATTGCGTAACAGAACTTTCT |
| | | | GTACATCACAGAAACAACAGGCAAACAATGGAGGATT |
| 479 | MELK | 3168526 | TGGCAGTATGATCACCTCACGGCTACCTATCTTCTGCTT |
| | | | CTAGCCAAGAAGGCTCGGGGAAAACCAGTTCGTTTAAG |
| | | | GCTTTCTTCTTTCTCCTGTGGACAAGCCAGTGCT |
| 480 | MELK | 3168528 | TGGAAGATGTGACCGCAAGTGATAAAAATTATGTGGCG |
| | | | GGATTAATAGACTATGATTGGTGTGAAGATGATTTATC |
| | | | AACAGGTGCTGCTACTCCCCGAACATCA |
| 481 | MELK | 3168529 | GGGGTGGAATCTAAATCATTAACTCCAGCCTTATGCAG |
| | | | AACACCTGCAAATAAATTAAAGAACAAAGAAAATGTAT |
| | | | ATACTCCTAAGTCTGCTGTAAAGAATGAAGAGTACTTT |
| | | | ATGTTTCCTGAGCCAAAGACTCCAGTTAATA |
| 482 | MELK | 3168530 | GAAACCAGTGCCTGAAAGAAACTCCAATTAAAATACCA |
| | | | GTAAATTCAACAGGAACAGACAAGTTAATGACAGGTGT |
| | | | CATTAGCCC |
| 483 | MELK | 3168531 | TGGATCTCAACCAAGCACATATGGAGGAGACTCCAAAA |
| | | | AGAAAGGGAGCCAAAGTGTTTGGGAGCCTTGAAAGGG |
| | | | GGTTGGATAAGGTTATCACTGTGCTCACCAGGAGCAAA |
| | | | AGGAAGGGTTCTGCCAGAGACGGGCCCAGAAGA |
| 484 | MELK | 3168533 | CTTCACTATAACGTGACTACAACTAGATTAGTGAATCC |
| | | | AGATCAACTGTTGA |
| 485 | MELK | 3168535 | ATACACTGAAGTGTCAAACACAGTCAGATTTTGGGAAA |
| | | | GTGACAATGCAATTTGAATTAGAAGTGTGCCAGCTTCA |
| | | | AAAA |
| 486 | MELK | 3168536 | CTTAAGGGCGATGCCTGGGTTTACAAAAGATTAGTGGA |
| | | | AGACATCCTATCTAGCTGCA |
| 487 | MELK | 3168537 | ATGGATTCTTCCATCCTGCCGGATGAGTGTGGGTGTGAT |
| | | | ACAGCCTACATAAAGACTGTTATGATCGCTTTGATTTTA |
| | | | AAGTTCATTGGAACTACCAACTTGTTTCTAAAGAGCTAT |
| | | | CTTAAGACCAATATCTCTTTGTTTTTAAACAAAAGATAT |
| | | | TATTTTGTGTATGAATCTAAATCAAGCCCATCTGTCATT |
| | | | ATGTTACTGTC |
| 488 | CDC20 | 3177818 | GAGGGGGCACCAGTGATCGACACATTTGCATCTGGAAC |
| | | | GTGTGCTCTGGGGCCTGTCTGAGTGCCGTGGATGCCC |
| 489 | CDC20 | 3177820 | TTATTTGGAAGTACCCAACCATGGC |
| 490 | BAG1 | 3203483 | CGGACCTATGTCCTACCTGTTCATGCAGTTGTCCATATC |
| | | | CAGTGGGATGTCCTAGGGGCTCAACTGTCCACAAACAT |
| | | | CACTGCCCCCCACAGAACTGGTACAGACCCCCAAACCA |
| | | | CCCATCTCAGCCCATGAAACCACTGGCCACCCAAGCCC |
| | | | ACAAATCAGAAACCAGTATGCTGCCTCCTTCCCCTCAG |
| | | | CCTTTACATACAGTCACATACCAAGCCTGTCAATTCCAC |
| | | | TCCCAATGTACCTCCTAACCATGAGTCTTTG |
| 491 | BAG1 | 3203484 | ACCTTCTCAGGAGCTGTAGTGTACCATTCAGGGACCTG |
| | | | GCACAGTCCATCCAGACATCCTGCTGAGCGTCGCCCAC |
| | | | ATATGCAGT |
| 492 | BAG1 | 3203486 | TCTTTCAGGTCCTCAGATGCACTGCACCCTCTCCTGCCT |
| | | | GGGGGTCTTTGCTCCTGCTACTACCTCTGCTTGAACAGC |
| | | | TCCTCACCTTCCTTCCTCCAACCCTACCCTTGTATAGGT |
| | | | GACTTTTGTTCATCCTTCAGAATTCAACTCACATGTCTC |
| | | | TTGCATGGAGAACCCTCACCTACTGTGTTGAGACCCTGT |
| | | | CCAGCCCCCAGGTGGGATCCTCTCTCGACTTCCCATACA |
| | | | TTTCTTTCACAGCATTTACATAGTCCATGATAGTTTACT |
| | | | TGTGGGATTATTTGGTTAATCTTTGCCTTTAACACCAGG |
| | | | GTTCCTTGGGTGAAGGAGCTTCTTTATC |
| 493 | BAG1 | 3203487 | AGAGTCGTTGAAGTCCCAGGAATTCAGGACTGGGCAGG |
| | | | TTAAGACCTCAGACAAGGTAGTAGAGGTAGACTTGTGG |
| | | | ACAAGGCTCGGGTCCCAGCCCACCGCACCCCAACTTTA |
| | | | ATCAGAGTGGTTCACTATTGATCTATTTTTGTGTGATAG |
| | | | CTGTGTGGCGTGGGCCACAACATTTAATGAGAAGTTAC |
| | | | TGTGCACCAAACTGCCGAACACCATTCTAAACTATTCAT |
| | | | ATATATTAGTCATTTAATTCTTACATAACTTGAGAGGTA |
| | | | GACAGATATCCTTATTTTAGAGATGAGGAAACCAAGAG |
| | | | AACTTAGGTCATTAGCGCAAGGTTGTAGAGTAAGCGGC |
| | | | AAAGCCAAGACACAAAGCTGGGTGGTTTGGTTTCAGAG |
| | | | CCAGTGCTTTTCCCCTCTACTGTACTGCCTCTCAACCAA |
| | | | CACAGGGTTGCACAGGCCCATTCTCTGATTTTTTTCCTC |
| | | | TTGTCCTCTGCCTCTCCCTCTAGCTCCCACTTCCTCTCTG |
| | | | CTCTAGTTCATTTTCTTTAGAGCAGCCCGAGTGATCATG |
| | | | AAGTGCAAATCTTGCCATGTCAGTCCCCTGCTTAGAACC |
| | | | CTCCAATGGCTCACTTTCTCTTTAGGCA |
| 494 | BAG1 | 3203488 | CTTCTGTCTCTGTGGTTGTACTGTCCAGCAATCCACCTT |
| | | | TTCTGGAGAGGGCCACCTCTGCCCAAATTTTCCCAGCTG |
| | | | TTTGGACCTCTGGGTGCTTTCTTTGGGCTGGTGAGAGCT |
| | | | CTAATTTGCCTTGGGCCAGTTTCAGGTTTATAGGCCCCC |
| | | | TCAGTCTTCAGATACATGAGGGCTTCTTTGCTCTTGTGA |
| | | | TCGTGTAGTCCCATA |
| 495 | BAG1 | 3203489 | TTTCCTACTCTCACACTGGTTCTCAATGAAAA |
| 496 | BAG1 | 3203490 | AATGGCGCCACCAGCTCTGCCGTCTCTGGAGCGGAATT |
| | | | TACCTGATTTCTTCAGGGCTGCTGGGGGCAACTGGCCAT |
| | | | TTGCCA |
| 497 | BAG1 | 3203493 | TTCAAAGACAGTAGATTGAAAAGGAAAGGCT |
| 498 | BAG1 | 3203495 | GAAGCTCTCTGCAAACTTGATAGGAGAGTAAAAGCCAC |
| | | | AATAGAGCAGTTTATGA |
| 499 | BAG1 | 3203496 | AGCTCTTGCTACGTGTTGTGAAATCTGTGTCTGTAGCTC |
| | | | GTGGCTGTGATAAACATCCTCAGAGATCCCTGCTATCC |
| | | | AACTCTTGTCACCAGCTAACTGGTCATTAGGCCAAGCT |
| | | | GTGTGAGTGAACCATTAGACAATTTGATAAAAATAAGC |
| | | | ACTGCTGCCATTCTTTAGCATGCGTCTGTGCCACAAGTT |
| | | | AGTGACACATGTACATATATTCCCTTTACCTCACAGTAA |
| | | | CCATCTAGGGTGTGGGCATCATAATCTTCATTTTTAAAA |
| | | | TGAGGGTACTGAGGCATGAATAGGCTAAATAATTTGCT |
| | | | CAAGATCACAAGTTAGTAAATGGCAGTCAGTTTTCAGG |
| | | | CGTAGGCTTTGAAGACTCTAAACCTGTGCTTTTAACATC |
| | | | TGCCTCTTCTTGTGACTGGGGGCTTCACTGGAGCCAAGC |
| | | | CTCAGGAGGCATTTGCAGCCACAGTGGGTCATGCGAGG |
| | | | TAGAGCAGACTGCAACCTGATAGAATTCTTGGACTGGC |
| | | | CCACGGCCAGCCTTTTTAATTTTTCCAAGAGTTAAAGTT |
| | | | GTGGATCTGAGATGTGGCCTGGCCTGCCAGGGCTATGG |
| | | | TGGGCTCAGTGTGATTGCATCATTAGTATTGTGGCATGG |
| | | | AGTCTTCCGTCAGCCTCAGAGA |
| 500 | BAG1 | 3203497 | AGTCCACAGGAAGAGGTTGAACTAAAGAAGTTGAAAC |
| | | | ATTTGGAGAAGTCTGTGGAGAAGATAGCTGACCAGCTG |
| | | | GAAGAGTTGAATAAAGAGCTTACTGGAATCCAGCA |
| 501 | BAG1 | 3203498 | CTAAATTTCTTGCTTCAGGGTCAGGGAGAATGGTGATG |
| | | | GGCAACAGGCAGAACCAAAGAGTCAAGTTTGAAGAAA |
| | | | CTATAGGACTTTTACTTGGAGCTGACACAGAGCGTATG |
| | | | GAGCGAAGTGTGAACTCAAGGAAGGCACCATGGCACA |
| | | | TGCCTATTGTCCCAGCAAGTTGGGAGTCTGAGACAGGA |
| | | | GGATCACTTGAGTCCAGGGGTTTGAGGCTGCAGTGAGT |
| | | | TATGATCATGCCTGTAAACAGCCACTGCACTCCAACCT |
| | | | GGGAACCGTAGTGAGACCCTATCTCAAAAAAGAAAAA |
| | | | AGTCTGAACTTAAGTCTAATCTACCTCTTTTGGACTGTG |
| | | | TGATCTCATGTTACTTTACTACATTAAGCCTCAGTTTCA |
| | | | TCATCTGTAAAACAGCAGTACTTCCCTGATGGAGTTGTG |
| | | | GTAAGGCTTAAAAAATAGGTAAGGTGCTTAGGATAGTG |
| | | | TGTGGCATGTAGGAAGTGTTCAATAAAAGTATTCATCG |
| | | | TTGTTAACCAGCATCACATTAAACAGGAGCGCTCAATT |
| | | | GGAGGCTGCCATTTAGGAATTCCATTTAAAGGAATGGT |
| | | | AGAATTCCACCTTTCTTGCCATTCTGGACTCCTCACAAG |
| | | | TGTTTACTG |
| 502 | BAG1 | 3203499 | GGAAATGGAAACACCGTTGTCAGCACTTGGAATACAAG |
| | | | ATGGTTGCCGGGT |
| 503 | BAG1 | 3203500 | GACCTGCTAGGTATTGGACACTCTCAAACACCTGGACA |
| | | | GGTGTCAGATTTGAAGTCTTATGATGATGGCAGAAATC |
| | | | TCACCATTTTCGATGAAGCTCATGGGCAGTCTTC |
| 504 | BAG1 | 3203501 | GAGAAGCACGACCTTCATGTTACCTCCCAGCAGGGCAG |
| | | | CAGTGAACCAGTTGTCCAAGACCTGGCCCAGGTTGTTG |
| | | | AAGAGGTCATAGGGGTTCCACAGTCTTTTC |
| 505 | BAG1 | 3203504 | GGCAGCTGGGCTCACCGTGACTGTCACCCAC |
| 506 | BAG1 | 3203505 | GAGGCGACCCAGGGCGAAGAGATGAATCGGAGCCAGG |
| | | | AGGTG |
| 507 | BAG1 | 3203506 | AGTTGACCCTGAGTGAGGAAGCGACCTGGAGTGAAGA |
| | | | GGCGACCCAGAGTGAG |
| 508 | BAG1 | 3203507 | ACCCGGCGCCGCTCGACCCGGAGCGAGGA |
| 509 | BAG1 | 3203508 | TGGGCGTCCACCTGCCCGGAGTACTGCCAGCGGGCATG |
| | | | ACCGACCCACCAGGGGCGCCGC |
| 510 | BAG1 | 3203509 | CTGGGTTCCCGGCTGCGCGCCCTTCGGCCAG |
| 511 | BAG1 | 3203510 | GCGGGGTTGTGAGACGCCGCGCTCAGCTTCCATCGCTG |
| | | | GGCGGTCAACAAGTGCG |
| 512 | CEP55 | 3258448 | TGTGACTCGGCCGACGCGAGCGCCGCGCTTCGCTTCAG |
| | | | CTGCT |
| 513 | CEP55 | 3258449 | GGAGGCGACCGCGGAGGGTGGCGAGGGGCGGCCAGGA |
| | | | CCCGCAGCCC |
| 514 | CEP55 | 3258450 | GGCAGATCGCGTCCGCGGGATTCAATCTCTGCCCGCTCT |
| | | | GATAACAGTCCTTTTCCCTGGCGCTCACTTCGTGCCTGG |
| | | | CACCCGGCTGGGCGCCTCAAGACCGTTGTCTCTTCGATC |
| | | | GCTTCTTTGGACTTGG |
| 515 | CEP55 | 3258451 | ATGTCTTCCAGAAGTACCAAAGATTTAATTA |
| 516 | CEP55 | 3258452 | TAAGTGGGGATCGAAGCCTAGTAACTCCAAATCCGAAA |
| | | | CTACATTAGAAAAATTAAAGGGAGAAATTGCACACTTA |
| | | | AAGACATCAGTGGATGAAATCACAAGTGGGAAAGGAA |
| | | | AGCTGACTGATAAAGAGAGACACAGACTT |
| 517 | CEP55 | 3258453 | GCGACTGAGAGACCAACTGAAGGCCAGATATAGTACTA |
| | | | CCACATTGCTTGAACAGCTGGAAGAGACAACGAGAGA |
| | | | AGGAGAAAGGAGGGAGCAGGTGTTGAAAGCCTTATCT |
| | | | GAAGAGAAAGACGTATTGAAACAACAGTTGTCTGCTGC |
| | | | AACCTCA |
| 518 | CEP55 | 3258454 | CTGTGGCTCCAAACTGCTTCAACTCATCAATAAATAAT |
| 519 | CEP55 | 3258455 | TCTGGAGAAAAATCAGCAGTGGCTCGTGTATGATCAGC |
| | | | AGCGGGAAGTCTATGTAAAAGGACTTTTAGCAAAGATC |
| | | | TTTGAGTTGGAAAAGAAAACGGAAACAGCTGCTCATTC |
| | | | ACTCCCACAGCAGACAAAAAAGC |
| 520 | CEP55 | 3258456 | GAAGCAGAAATGTTACAACGATCTCTTGGCAAGTGCAA |
| | | | AAAAAGATCTTGAGGTTGAACGACAAACCATAACTCAG |
| 521 | CEP55 | 3258457 | AGCTGTTGTATTCACAAAGAAGGGCAGATGTGCAACAT |
| | | | CTGGAAGATGATAGGCATAAAACAGAGAAGATACAAA |
| | | | AACTCAGGGAAGAGAATGATATTGCTAGGGGAAAACTT |
| | | | GAAGAAGAGAAGAAGAGATCCGAAGAGCTCTTATCTC |
| 522 | CEP55 | 3258459 | CTTTACACATCTCTGCTAAAGCAGCAAGAAGAACAAAC |
| | | | AAGGGTAGCTCTGTTGGAACAACAG |
| 523 | CEP55 | 3258460 | ACTCATTCGGGTTGCTTCTAAATTCAATTCTGCCTGTTA |
| | | | GAAAATGCAGTTTTTCCTCATGTTTATGCTGTTCTATGG |
| | | | AGAACTGTTTGAAAGTTGTGAAAAGTGTCT |
| 524 | CEP55 | 3258461 | TGAAAAACTCGACCGTCAACATGTGCAGCATCAATTGC |
| | | | ATGTAATTCTTAAGGAGCTCCGAAAAGCAAGAAATCAA |
| | | | ATAACACAGTTGGAATCCTTG |
| 525 | CEP55 | 3258462 | TCCGAACTTAGGAGGATACAGCTTAACACACAGCTAGC |
| | | | TGTATCTCAAATCAGTAGGTAGAGCCTCTGCCTCATTTG |
| | | | AAGCAACTGCCCTTTGAGCATCAATTCAGAGGACATGA |
| | | | AAGAGGGACATGATCACATCTGGAAACA |
| 526 | CEP55 | 3258463 | CAGAGCCATTAGTCACTTTCCAAGGAGAGACTGAAAAC |
| | | | AGAGAAAAAGTTGCCGCCTCACCAAAAAGTCCCACTGC |
| | | | TGCACTCAATGAAAGCCTGGTGGAATGTCCCAAGTGCA |
| | | | ATATACAGTATCCAGCCACTGAGCATCGCGATCTGCTT |
| | | | GTCCATG |
| 527 | CEP55 | 3258464 | TACCTTTGACACTCCAGCATGCTAGTGAATCATGTATCT |
| | | | TTTAGGCTGCTGTGCATTTCTCTTGGCAGTGATACCTCC |
| | | | CTGACATGGTTCATCATCAGGCTGCAATGACAGAATGT |
| | | | GGTGAGCAGCGTCTACTGAGACTACTAACATTTTGCAC |
| | | | TGTCAAAATACTTGGTGAGGAAAAGATAGCTCAGGTTA |
| | | | TTGCTAATGGGTTAATGCACCAGCAAGCAAAATATTTT |
| | | | ATGTTTTGGGGGTTTTGAAAAATCAAAGATAATTAACC |
| | | | AAGGATCTTAACTGTGTTCGCATTTTTTATCCAAGCACT |
| | | | TAGAAAACCTACAATCCTAATTTTGATGTCCATTGTTAA |
| | | | GAGGTGGTGATAGATACTATTTTTTTTTTCATATTGTAT |
| | | | AGCGGTTATTAGAAAAGTTGGGGATTTTCTTGATCTTTA |
| | | | TTGCTGCTTACCATTGAAACTTAACCCAGCTGTGTTCCC |
| | | | CAACTCTGTTCTGCGCACGAAACAGTATCTG |
| 528 | CEP55 | 3258465 | TCAGATCTTTGTTTGTCTGAACAGGTATTTTTATACATG |
| | | | CTTTTTGTAAACCAAAAACTTTTAAATTTCTTCAGGTTT |
| | | | TCTAACATGCTTACCACTGGGCTACTGTAAATGAGAAA |
| 529 | MKI67 | 3312496 | ACTCGTGAGCACATCTTTAGGGACCAAGAGTGACTTTC |
| | | | TGTAAGGAGTGACTCGTGGCTTGCCTTGGTCTCTTGGGA |
| | | | ATACTTTTCTAACTAGGGTTGCTCTCACCTGAGACATTC |
| | | | TCCACCCGCGGAATCTCAGGGTCCCAGGCTGTGGGCCA |
| | | | TCACGACCTCAAACTGGCTCCTAATCTCCAGCTTTCCTG |
| | | | TCATTGAAAGCTTCGGAAGTTTACTGGCTCTGCTCCCGC |
| | | | CTGTTTTCTTTCTGACTCTATCTGGCAGCCCGATGCCAC |
| | | | CCAGTACAGGAAGTGACACCAGTACTCTGTAAAGCATC |
| | | | ATCATCCTTGGAGAGACTGAGCACTCAGCACCTTCAGC |
| | | | CACGATTTCAGGATCGCTTCCTTGTGAGCCGCTGCCTCC |
| | | | GAAATCTCCTTTGAAGCCCAGACATCTTTCTCCAGCTTC |
| | | | AGACTTGTAGATATAACTCGTTCATCTTCATTTACTTTC |
| | | | CACTTTGCCCCCTGTCCTCTCTGTGTTCCCCAAATCAGA |
| | | | GAATAGCCCGCCATCCCCCAGGTCACCTGTCTGGATTCC |
| | | | TCCCCATTCACCCACCTTGCCAGGTGCAGGTGAGGATG |
| | | | GTGCACCAGACAGGGTAGCTGTCCCCCAAAATGTGCCC |
| | | | TGTGCGGGCAGTGCCCTGTCTCCACGTTTGTTTCCCCAG |
| | | | TGTCTGGCGGGGAGCCAGGTGACATCATAAATACTTGC |
| | | | TGAATGAATGCAGAAATCAGCGGTACTGACTTGTACTA |
| | | | TATTGGCTGCCATGATAGGGTTCTCACAGCGTCATCCAT |
| | | | GATCGTAAGGGAGAATGACATTCTGCTTGAGGGAGGGA |
| | | | ATAGAAAGGGGCAGGGAGGGGACATCTGAGGGCTTCA |
| | | | CAGGGCTGCAAAGGGTACAGGGATTGCACCAGGGCAG |
| | | | AACAGGGGAGGGTGTTCAAGGAAGAGTGGCTCTTAGCA |
| | | | GAGGCACTTTGGAAGGTGTGAGGCATAAATGCTTCCTT |
| | | | CTACGTAGGCCAACCTCAAAACTTTCAGTAGGAATGTT |
| | | | GCTATGATCAAGTTGTTCTAACACTTTAGACTTAGTAGT |
| | | | AATTATGAACCTCACATAGAAAAATTTCATCCAGCCAT |
| | | | ATGCCTGTGGAGTGGAATATTCTGTTTAGTAGAAAAAT |
| | | | CCTTTAGAGTTCAGCTCTAACCAGAAATCTTGCTGAAGT |
| | | | ATGTCAGCACCTTTTCTCACCCTGGTAAGTACAGTATTT |
| | | | CAAGAGCACGCTAAGGGTGGTTTTCATTTTACAGGGCT |
| | | | GTTGATGATGGGTTAAAAATGTTCATTTAAGGGCTACC |
| | | | CCCGTGTTTAATAGATGAACACCACTTCTACACAACCCT |
| | | | CCTTGGTACTGGGGGAGGGAGAGATCTGACAAATACTG |
| | | | CCCATTCCCCTAGGCTGACTGGATTTGAGAACA |
| 530 | MKI67 | 3312497 | ATGAGCGCACGGATGAATGGAGCTTACAAGATCTGTCT |
| | | | TTCCAATGGCCGGGGGCATTTGGTCCCCAAATTAAGGC |
| | | | TATTGGACATCTGCACAGGACAGTCCTATTTTTGATGTC |
| 531 | MKI67 | 3312498 | AGGAACAACTATCCTCGTCTGTCCCAACACTGAGCAGG |
| | | | CACTCGGTAAAC |
| 532 | MKI67 | 3312499 | ATTTGCTGGGTCTGAATCGGCTTCATAAACTCCACTGGG |
| | | | AGCACTGCTGGGCTCCTGGACTGAGAATAGTTGAACAC |
| | | | CGGGGGCTTTGTGAAGGAGTCTGGGCCAAGGTTTGCCC |
| | | | TCAGCTTTGCAGAATGAAGCCTTGAGGTCTGTCACCAC |
| | | | CCACAGCCACCCTACAGCAGCCTTAACTGTGACACTTG |
| | | | CCACACTGTGTCGTCGTTTGTTTGCCTATGTCCTCC |
| 533 | MKI67 | 3312500 | GGACAATGTGTGTGTCAAGAAAATAAGAACCAGAAGTC |
| | | | ATAGGGACAGTGAAGATATTTG |
| 534 | MKI67 | 3312502 | CAGAAGAGTGCGAAGGTTCTCATGCAGAATCAGAAAG |
| | | | GGAAAGGAGAAGCAGGAAATTCAGACTCCATGTGCCTG |
| | | | AGATCAAGAAAGACAAAAAGCCAGCCTGCAGCAAGCA |
| | | | CTTTGGAGAGCAAATCTGTGCAGAGAGTAACGCGGAGT |
| | | | GTCAA |
| 535 | MKI67 | 3312503 | GAGCCCGGAAACCCATACCTAGAGACAAAG |
| 536 | MKI67 | 3312504 | TATCCCTGCGCTCCAGACGCCAAAAT |
| 537 | MKI67 | 3312506 | AAGAGGCTGCGCTGCATGCCAGCACCAGAGGAAATTGT |
| | | | GGAGGAGCTGCCAGCCAGCAAGAAGCAGAGGGTTGCT |
| | | | CCCAGGGCAAGAGGCAAATCATCCGAACCCGTGGTCAT |
| | | | CATGAAGAGAAGTTTGAGGACTTCTGCAAAAAGAATTG |
| | | | AACCTGCGGAAGAGCTGAACAGCAACGACATGAAAAC |
| | | | CAACAAAGAGGAACACAAATTACAAGACTCGGTCCCTG |
| 538 | MKI67 | 3312507 | CCCGTGCTCTAGAAGACCTGGTTGACTTCAAAGAGCTC |
| | | | TTCTCAGCACCAGGTCACACTGAAGAGTCAATGACTAT |
| | | | TGACAAAAACACAAAAATTCCCTGCAAATCTCCCCCAC |
| | | | CAGAACTAACAGACACTGCCACGAGCACAAAGAGATG |
| | | | CCCCAAGACACGTCCCAGGAAAGAAGTAAAAGAGGAG |
| | | | CTCTCAGCAGTTGAGAGGCTCACGCAAACATCAGGGCA |
| | | | AAGCACACACACACACAAAGAACCAGCAAGCGGTGAT |
| | | | GAGGGCATCAAAGTATTGAAGCAACGTGCAAAGAAGA |
| | | | AACCAAACCCAGTAGAAGAGGAACCCAGCAGGAGAAG |
| | | | GCCAAGAGCACCTAAGGAAAAGGCCCAACCCCTGGAA |
| | | | GACCTGGCCGGCTTCACAGAGCTCTCTGAAACATCAGG |
| | | | TCACACTCAGGAATCACTGACTGCTGGCAAAGCCACTA |
| | | | AAATACCCTGCGAATCTCCCCCACTAGAAGTGGTAGAC |
| | | | ACCACAGCAAGCACAAAGAGGCATCTCAGGACACGTGT |
| | | | GCAGAAGGTACAAGTAAAAGAAGAGCCTTCAGCAGTC |
| | | | AAGTTCACACAAACATCAGGGGAAACCACGGATGCAG |
| | | | ACAAAGAACCAGCAGGTGAAGATAAAGGCATCAAAGC |
| | | | ATTGAAGGAATCTGCAAAACAGACACCGGCTCCAGCAG |
| | | | CAAGTGTAACTGGCAGCAGGAGACGGCCAAGAGCACC |
| | | | CAGGGAAAGTGCCCAAGCCATAGAAGACCTAGCTGGCT |
| | | | TCAAAGACCCAGCAGCAGGTCACACTGAAGAATCAATG |
| | | | ACTGATGACAAAACCACTAAAATACCCTGCAAATCATC |
| | | | ACCAGAACTAGAAGACACCGCAACAAGCTCAAAGAGA |
| | | | CGGCCCAGGACACGTGCCCAGAAAGTAGAAGTGAAGG |
| | | | AGGAGCTGTTAGCAGTTGGCAAGCTCACACAAACCTCA |
| | | | GGGGAGACCACGCACACCGACAAAGAGCCGGTAGGTG |
| | | | AGGGCAAAGGCACGAAAGCATTTAAGCAACCTGCAAA |
| | | | GCGGAAGCTGGACGCAGAAGATGTAATTGGCAGCAGG |
| | | | AGACAGCCAAGAGCACCTAAGGAAAAGGCCCAACCCC |
| | | | TGGAAGATCTGGCCAGCTTCCAAGAGCTCTCTCAAACA |
| | | | CCAGGCCACACTGAGGAACTGGCAAATGGTGCTGCTGA |
| | | | TAGCTTTACAAGCGCTCCAAAGCAAACACCTGACAGTG |
| | | | GAAAACCTCTAAAAATATCCAGAAGAGTTCTTCGGGCC |
| | | | CCTAAAGTAGAACCCGTGGGAGACGTGGTAAGCACCAG |
| | | | AGACCCTGTA |
| 539 | MKI67 | 3312508 | GGAGAACTCTTAGCGTGCAGGAATCTAATGCCATCAGC |
| | | | AGGCAAAGCCATGCACACGCCTAAACCATCAGTAGGTG |
| | | | AAGAGAAAGACATCATCATATTTGTGGGAACTCCAGTG |
| | | | CAGAAACTGGACCTGACAGAGAACTTAACCGGCAGCA |
| | | | AGAGACGGCCACAAACTCCTAAGGAAGAGGCCCAGGC |
| | | | TCTGGAAGACCTGACTGGCTTTAAAGAGCTCTTCCAGA |
| | | | CCCCTGGTCATACTGAAGAAGCAGTGGCTGCTGGCAAA |
| | | | ACTACTAAAATGCCCTGCGAATCTTCTCCACCAGAATC |
| | | | AGCAGACACCCCAACAAGCACAAGAAGGCAGCCCAAG |
| | | | ACACCTTTGGAGAAAAGGGACGTACAGAAGGAGCTCTC |
| | | | AGCCCTGAAGAAGCTCACACAGACATCAGGGGAAACC |
| | | | ACACACACAGATAAAGTACCAGGAGGTGAGGATAAAA |
| | | | GCATCAACGCGTTTAGGGAAACTGCAAAACAGAAACTG |
| | | | GACCCAGCAGCAAGTGTAACTGGTAGCAAGAGGCACCC |
| | | | AAAAACTAAGGAAAAGGCCCAACCCCTAGAAGACCTG |
| | | | GCTGGCTTGAAAGAGCTCTTCCAGACACCAGTATGCAC |
| | | | TGACAAGCCCACGACTCACGAGAAAACTACCAAAATAG |
| | | | CCTGCAGATCACAACCAGACCCAGTGGACACACCAACA |
| | | | AGCTCCAAGCCACAGTCCAAGAGAAGTCTCAGGAAAGT |
| | | | GGACGTAGAAGAAGAATTCTTCGCACTCAGGAAACGAA |
| | | | CACCATCAGCAGGCAAAGCCATGCACACACCCAAACCA |
| | | | GCAGTAAGTGGTGAGAAAAACATCTACGCATTTATGGG |
| | | | AACTCCAGTGCAGAAACTGGACCTGACAGAGAACTTAA |
| | | | CTGGCAGCAAGAGACGGCTACAAACTCCTAAGGAAAA |
| | | | GGCCCAGGCTCTAGAAGACCTGGCTGGCTTTAAAGAGC |
| | | | TCTTCCAGACACGAGGTCACACTGAGGAATCAATGACT |
| | | | AACGATAAAACTGCCAAAGTAGCCTGCAAATCTTCACA |
| | | | ACCAGACCCAGACAAAAACCCAGCAAGCTCCAAGCGA |
| | | | CGGCTCAAGACATCCCTGGGGAAAGTGGGCGTGAAAG |
| | | | AAGAGCTCCTAGCAGTTGGCAAGCTCACACAGACATCA |
| | | | GGAGAGACTACACACACACACACAGAGCCAACAGGAG |
| | | | ATGGTAAGAGCATGAAAGCATTTATGGAGTCTCCAAAG |
| | | | CAGATCTTAGACTCAGCAGCAAGTCTAACTGGCAGCAA |
| | | | GAGGCAGCTGAGAACTCCTAAGGGAAAGTCTGAAGTCC |
| | | | CTGAAGACCTGGCCGGCTTCATCGAGCTCTTCCAGACA |
| | | | CCAAGTCACACTAAGGAATCAATGACTAACGAAAAAAC |
| | | | TACCAAAGTATCCTACAGAGCTTCACAGCCAGACCTAG |
| | | | TGGACACCCCAACAAGCTCCAAGCCACAGCCCAAGAGA |
| | | | AGTCTCAGGAAAGCAGACACTGAAGAAGAATTTTTAGC |
| | | | ATTTAGGAAACAAACGCCATCAGCAGGCAAAGCCATGC |
| | | | ACACACCCAAACCAGCAGTAGGTGAAGAGAAAGACAT |
| | | | CAACACGTTTTTGGGAACTCCAGTGCAGAAACTGGACC |
| | | | AGCCAGGAAATTTACCTGGCAGCAATAGACGGCTACAA |
| | | | ACTCGTAAGGAAAAGGCCCAGGCTCTAGAAGAACTGAC |
| | | | TGGCTTCAGAGAGCTTTTCCAGACACCATGCACTGATA |
| | | | ACCCCACGACTGATGAGAAAACTACCAAAAAAATACTC |
| | | | TGCAAATCTCCGCAATCAGACCCAGCGGACACCCCAAC |
| | | | AAACACAAAGCAACGGCCCAAGAGAAGCCTCAAGAAA |
| | | | GCAGACGTAGAGGAAGAATTTTTAGCATTCAGGAAACT |
| | | | AACACCATCAGCAGGCAAAGCCATGCACACGCCTAAAG |
| | | | CAGCAGTAGGTGAAGAGAAAGACATCAACACATTTGTG |
| | | | GGGACTCCAGTGGAGAAACTGGACCTGCTAGGAAATTT |
| | | | ACCTGGCAGCAAGAGACGGCCACAAACTCCTAAAGAA |
| | | | AAGGCCAAGGCTCTAGAAGATCTGGCTGGCTTCAAAGA |
| | | | GCTCTTCCAGACACCAGGTCACACTGAGGAATCAATGA |
| | | | CCGATGACAAAATCACAGAAGTATCCTGCAAATCTCCA |
| | | | CAACCAGACCCAGTCAAAACCCCAACAAGCTCCAAGCA |
| | | | ACGACTCAAGATATCCTTGGGGAAAGTAGGTGTGAAAG |
| | | | AAGAGGTCCTACCAGTCGGCAAGCTCACACAGACGTCA |
| | | | GGGAAGACCACACAGACACACAGAGAGACAGCAGGAG |
| | | | ATGGAAAGAGCATCAAAGCGTTTAAGGAATCTGCAAAG |
| | | | CAGATGCTGGACCCAGCAAACTATGGAACTGGGATGGA |
| | | | GAGGTGGCCAAGAACACCTAAGGAAGAGGCCCAATCA |
| | | | CTAGAAGACCTGGCCGGCTTCAAAGAGCTCTTCCAGAC |
| | | | ACCAGACCACACTGAGGAATCAACAACTGATGACAAA |
| | | | ACTACCAAAATAGCCTGCAAATCTCCACCACCAGAATC |
| | | | AATGGACACTCCAACAAGCACAAGGAGGCGGCCCAAA |
| | | | ACACCTTTGGGGAAAAGGGATATAGTGGAAGAGCTCTC |
| | | | AGCCCTGAAGCAGCTCACACAGACCACACACACAGACA |
| | | | AAGTACCAGGAGATGAGGATAAAGGCATCAACGTGTTC |
| | | | AGGGAAACTGCAAAACAGAAACTGGACCCAGCAGCAA |
| | | | GTGTAACTGGTAGCAAGAGGCAGCCAAGAACTCCTAAG |
| | | | GGAAAAGCCCAACCCCTAGAAGACTTGGCTGGCTTGAA |
| | | | AGAGCTCTTCCAGACACCAATATGCACTGACAAGCCCA |
| | | | CGACTCATGAGAAAACTACCAAAATAGCCTGCAGATCT |
| | | | CCACAACCAGACCCAGTGGGTACCCCAACAATCTTCAA |
| | | | GCCACAGTCCAAGAGAAGTCTCAGGAAAGCAGACGTA |
| | | | GAGGAAGAATCCTTAGCACTCAGGAAACGAACACCATC |
| | | | AGTAGGGAAAGCTATGGACACACCCAAACCAGCAGGA |
| | | | GGTGATGAGAAAGACATGAAAGCATTTATGGGAACTCC |
| | | | AGTGCAGAAATTGGACCTGCCAGGAAATTTACCTGGCA |
| | | | GCAAAAGATGGCCACAAACTCCTAAGGAAAAGGCCCA |
| | | | GGCTCTAGAAGACCTGGCTGGCTTCAAAGAGCTCTTCC |
| | | | AGACACCAGGCACTGACAAGCCCACGACTGATGAGAA |
| | | | AACTACCAAAATAGCCTGCAAATCTCCACAACCAGACC |
| | | | CAGTGGACACCCCAGCAAGCACAAAGCAACGGCCCAA |
| | | | GAGAAACCTCAGGAAAGCAGACGTAGAGGAAGAATTT |
| | | | TTAGCACTCAGGAAACGAACACCATCAGCAGGCAAAGC |
| | | | CATGGACACACCAAAACCAGCAGTAAGTGATGAGAAA |
| | | | AATATCAACACATTTGTGGAAACTCCAGTGCAGAAACT |
| | | | GGACCTGCTAGGAAATTTACCTGGCAGCAAGAGACAGC |
| | | | CACAGACTCCTAAGGAAAAGGCTGAGGCTCTAGAGGAC |
| | | | CTGGTTGGCTTCAAAGAA |
| 540 | MKI67 | 3312509 | AACAACAGTTGAAGGCATCCCTGGGGAAAGTAGGTGTG |
| | | | AAAGAAGAGCTCCTAGCAGTCGGCAAGTTCACACGGAC |
| | | | GTCAGGGGAGACCACGCACACGCACAGAGAGCCAGCA |
| | | | GGAGATGGCAAGAGCATCAGAACGTTTAAGGAGTCTCC |
| | | | AAAGCAGATCCTGGACCCAGCAGCCCGTGTAACTGGAA |
| | | | TGAAGAAGTGGCCAAGAACGCCTAAGGAAGAGGCCCA |
| | | | GTCACTAGAAGACCTGGCTGGCTTCAAAGAGCTCTTCC |
| | | | AGACACCAGGTCCCTCTGAGGAATCAATGACTGATGAG |
| | | | AAAACTACCAAAATAGCCTGCAAATCTCCACCACCAGA |
| | | | ATCAGTGGACACTCCAACAAGCACAAAGCAATGGCCTA |
| | | | AGAGAAGTCTC |
| 541 | MKI67 | 3312510 | TGCAAACAGGTCAGGAAGGTCTACAGAGTTCAGGAATA |
| | | | TACAGAAGCTACCTGTGGAAAGTAAGAGTGAAGAAAC |
| | | | AAATACAGAAATTGTTGAGTGCATCCTAAAAAGAGGTC |
| | | | AGAAGGCAACACTACTACAACAAAGGAGAGAAGGAGA |
| | | | GATGAAGGAAATAGAAAGACCTTTTGAGACATATAAGG |
| | | | AAAATATTGAATTAAAAGAAAACGATGAAAAGATGAA |
| | | | AGCAATGAAGAGATCAAGAACTTGGGGGCAGAAATGT |
| | | | GCACCAATGTCTGACCTGACAGACCTCAAGAGCTTGCC |
| | | | TGATACAGAACTCATGAAAGACACGGCACGTGGC |
| 542 | MKI67 | 3312511 | CCCAGTGAAGGAGCAACCGCAGTTGACAAGCACATGTC |
| | | | ACATCGCTATTTCAAATTCAGAGA |
| 543 | MKI67 | 3312513 | TAAAAACGTAGTCTTAGATCTTATAAATCTTTTGACTCT |
| | | | ACTGTTTTTTACTGTGTTAATGTTTGTTTTGCTAACTTTG |
| | | | TTTATCTGCTG |
| 544 | MKI67 | 3312514 | CGCAAACTCTCCTTGTACCATAATAATAGGGAAAGCTC |
| | | | ATACTGAAAAAGTACATGTGCCTGCTCGACCCTACAGA |
| | | | GTGCTCAA |
| 545 | MKI67 | 3312515 | AGCCTGTGGGCGAAGTTCACAGTCAA |
| 546 | MKI67 | 3312516 | CATGGGCAGATGTAGTAAAACTTGGTGCAAAACAAACA |
| | | | CAAACTAAAGTCATAAAACATGGTCCTCAAAGGTC |
| 547 | MKI67 | 3312517 | AAGAGAGTGTCTATCAGCCGAAGTCAACATGATATTTT |
| | | | ACAGATGATATGTTCCAAAAGAAGAAGTGGTGCTTCGG |
| | | | AAGCAAAT |
| 548 | MKI67 | 3312518 | AAACAAGAGTCAGGTTCAGAAATCCATGTGGAAGTGAA |
| | | | GGCACAAAGCTTGGTTATAAGCCCTCCAGCTCCTAGTC |
| | | | CTAGGAAAACTCCAGTTGCCAGTGATCAACGCCGTAGG |
| | | | TC |
| 549 | MKI67 | 3312519 | CCTTTGAAAAGAAGGCGTGTGTCCTTTGGTGGGCACCT |
| | | | AAGACCTGAACTATTTGATGAAAACTTGCCTCCTAATA |
| | | | CGCCTCTCAAAAGGGGAGAAGCCCCAACCAAAAGAAA |
| | | | GTCTCTGGTAATGCACACTCC |
| 550 | MKI67 | 3312520 | GGACAGATGTGCTCTGGGTTACCTGGTCTTAGTTCAGTT |
| | | | GATATCAACAACTTTGGTGATTCCATT |
| 551 | MKI67 | 3312521 | TTGAGAGGAAGATCCAAAAGGATTCCCTCAG |
| 552 | MKI67 | 3312522 | GAAGCTTTCAACTAGAAATCGAACACCAGCTAAAGTTG |
| | | | AAGATGCAGCTGACTCTGCCACTAAGCCAGAAAATCTC |
| | | | TCTTCCAAAACCAGAGGAAGTATTCCTACAGATGTGGA |
| | | | AGTTCTGCCTACGGAAACTGAAATTCACAATGAGC |
| 553 | MKI67 | 3312523 | CCAGCGTTAAATTAGTGAGCCGTTATGGAGAATTGAAG |
| | | | TCTGTTCCCACTACACAATGTCTTGACAATAGCAAAAA |
| | | | AAATGAATCTCCCTTTTGGAAGCTTTATGAGTCAGTGAA |
| | | | GAAAGAGTTGGATGTAAAATCACAAAAAGAAAATGTC |
| | | | CTACAGTATTGTAGAAAATCTGGATTACAAACTGATTA |
| | | | CGCAACAGAGAAAGAAAGTGCTGATGGTTTACAGGGG |
| | | | GAGACCCAACTGTTGGTCTCGCGTAAGTCAAGACCAAA |
| | | | ATCTGGTGGGAGCGGCCACGCTGTGGCAGAGCCTGCTT |
| | | | CACCTGAACAAGAGCTTGACCAGAACAAGGGGAAGGG |
| | | | AAGAGACGTGGAGTCTGTTCAGACTCCCAGCAAGGCTG |
| | | | TGGGCGCCAGCTTTCCTCTCTATGAGCCGGCTAAAATG |
| 554 | MKI67 | 3312524 | AGCCAGCACGTCGTGTCTCAAGATCTAGCTTCT |
| 555 | MKI67 | 3312526 | TTCAGAATGGAAGGAAGTCAACTGAATTTC |
| 556 | MKI67 | 3312527 | CCAACACAAGTAAATGGGTCTGTTATTGATGAGCCTGT |
| | | | ACGGCTAAAACATGGAGATGTAATAACTATTATTGATC |
| | | | GTTCCTTCAG |
| 557 | MKI67 | 3312528 | TGTGACATCCGTATCCAGCTTCCTGTTGTGTCAAAACAA |
| | | | CATT |
| 558 | MKI67 | 3312530 | CCTGAGCCTCAGCACCTGCTTGTTTGGAAG |
| 559 | MKI67 | 3312531 | CCCACGAGACGCCTGGTTACTATCAA |
| 560 | MKI67 | 3312532 | TTTGCTTCTGGCCTTCCCCTACGGATTATACCTGGCCTT |
| | | | CCCCTACGGATTATACTCAACTTACTGTTTAGA |
| 561 | MKI67 | 3312533 | GACTCGGTGGGAGCCGCTAGAGCCGGGCGCCCGGGGA |
| | | | CGTAGCCTGTAGGGCCACCGGGTCCCCGTCAGAGGCGG |
| | | | CGGCGGGAGCAGCGGGGACTGCAGGCCGGGGTGCAGC |
| | | | GAACGCGACCCCGCGGGCTGCGGCCCGGTGTGTGCGGA |
| | | | GCGTGGCGGGCGCAGCTTACCGGGCGGAGGTGAGCGC |
| | | | GGCGCCGGCTCCTCCTGCGGCGGACTTTGGGTGCGACT |
| | | | TGACGAGCGGTGGTTCGACAAGTGGCCTTGCGGGCCGG |
| | | | AT |
| 562 | TMEM45B | 3356039 | TGCAGACGGCTGCGAGGCGCTGGGC |
| 563 | TMEM45B | 3356044 | GCAGGGCTGAGACTATCTTCTGCTCAGGAA |
| 564 | TMEM45B | 3356053 | TCTCATGTTTTTCTATAAGCAGTTAAGAGAAGCCACACA |
| | | | GCATCCTGAACACTTTGCTTTCT |
| 565 | TMEM45B | 3356054 | ATGGCAAATTTCAAGGGCCACGCGCTTCCAGGGAGTTT |
| | | | CTTCCTGATCATTGGGCTGTGTTGGTCAGTGAAGTACCC |
| | | | GCT |
| 566 | TMEM45B | 3356055 | GTACTTTAGCCACACGCGGAAGAACAGCCCACTACATT |
| | | | ACTATCAG |
| 567 | TMEM45B | 3356056 | CGTCTCGAGATCGTCGAAGCCGCAATTAGGACTTTGTTT |
| | | | TCCGTCACTG |
| 568 | TMEM45B | 3356057 | GTCATTTGGTCTAGGGAATCTCCTCATCATACCCAGAAC |
| | | | CTTTAATTCATTTTCTGAGCCCTGTGAAATAGATGTTCC |
| | | | CACTGGCAGAGATAATAGGGCAACAATTTCCTGATGGC |
| | | | CACTAGACTATTTTATCGTAACATCCATTGTGTACAGAG |
| | | | CTTTATAATACTAACGGTTGACAGCTCTCACATCATG |
| 569 | TMEM45B | 3356058 | CACCATGTACCTATTCTTTGCAGTCTCAGGAATTGTTGA |
| | | | CATGCTCACCTATCTGGTCAGCCACGTTCCCTTGG |
| 570 | TMEM45B | 3356061 | AACCGGCCTCCGCTGGACCAGCACATCCACTCACTCCT |
| | | | GCTGTATGCTCTGTTCGGAGGGTGTGTTAGTATCTCCCT |
| | | | AGAGGTGATCTTCCGGGACCACATTGTGCTGGAACTTTT |
| | | | CCGAACCAGTCTCATC |
| 571 | TMEM45B | 3356063 | ATTGGGTTTGTGCTGTTCCCACCTTTTGGAACACCCGAA |
| | | | TGGGACCAGAAGGATGATGCCAACCTCATGTTCATCAC |
| | | | CATGTGCTTCTGCTGGCACTACCTGGCTGCCCTCAGCAT |
| | | | TGTGGCCGTCAACTATTCTCTTG |
| 572 | TMEM45B | 3356065 | CCTTTTGACTCGGATGAAGAGACACGGAAGGGGAGAA |
| | | | ATCATTGGAATTCAGAAGCTGAATTCAGATGACACTTA |
| | | | CCAGACCGCCCTCTTGAGTGGCTCAGA |
| 573 | TMEM45B | 3356066 | TAAAGTCTGTGTTGGTATAGTACCCTTCATAAGGAAAA |
| | | | ATGAAGTAATGCCTATAAGTAGCAGGCCTTTGTGCCTC |
| | | | AGTGTCAAGAGAAATCAAGAGATGCTAAAAGCTTTACA |
| | | | ATGGAAGTGGCCTCATGGATGAATCCGGGGTATGAGCC |
| | | | CAGGAGAACGTGCTGCTTTTGGTAACTTATCCCTTTTTC |
| | | | TCTTAAGAAAGCAGGTACTTTCTTATTAGAAATATGTTA |
| | | | GAATGTGTAAGCAAACGACAGTGCCTTTAGAATTACAA |
| | | | TTCTAACTTACATATTTTTTGAAAGTAAAATAATTCACA |
| | | | AGCTTTGGTATTTTAAAATTATTGTTAAACATATCATAA |
| | | | CTAATCATACCAGGGTACTGCAATACCACTGTTTATAA |
| | | | GTGACAAAATTAGGCCAAAGGTGATTTTTTTTTAAATCA |
| | | | GGAAGCTGGTTACTGGCTCTACTGAGAGTTGGAGCCCT |
| | | | GATGTTCTGATTCTTCAAAGTCACCCTAAAAGAAGATCT |
| | | | GACAGGAAAGCTGTATAATGAGATAGAAAAACGTCAG |
| | | | GTATGGAAGGCTTTCAGTTTTAATATGGCTGAAAGCAA |
| | | | AGGATAACGAATTCAGAATTAGTAATGTAAAATCTTGA |
| | | | TACCCTAATCTTGCTTCTGGATCTGTTCTTTTTTTAAAAA |
| | | | AACTTCCTTCACCGCGCCTATAATCCTAGCACTTTGGGA |
| | | | GGCCGAGGCAGGCAGATCACGGGGTCAGGAGATCAAG |
| | | | ACCATCCTGGCTAACATGGTGAAACCCCGTCTCTACTG |
| | | | AAAATACAAAAAATTAGCCGGGTGTGGTGGCGGGCGCC |
| | | | TGTAGTTCCAGCTACTCGGGAGGCTGAGGCAAGAGAAT |
| | | | GGCATGAACCCGGTAGGGGAGCTTGCAGTGAGCCCAGA |
| | | | TCATGCCACTGTACTCCAGCCTAGGTGACAGAGCAAGA |
| | | | CTCTGTCTCAAAAACAAGCAAACAGACTTCCTTCAACA |
| | | | AATATTTATTAAATATCCACTTTGCAACAGCACTGAAAT |
| | | | GGCTGTAAGGACTCCTGAGATATGTGTCCAGCAAGG |
| 574 | PGR | 3388371 | AGTGTTCCCGTCTTCTCCGAGCTTAGAGTTGGATGGGGA |
| | | | ATAAAGACAGGTAAACAGATAGCTACAATATTGTACTG |
| | | | TGAATGCTTATGCTGGAGGAAGTACAGGGAACTATTGG |
| | | | AGCACCTAAGAGGAGCACCTACCTTGAATTTAGGGGTT |
| | | | AGCAGAGGCATCCTGAAAAAAGTCAAAGCTAAGCCAC |
| | | | AATCTATAAGCAGTTTAGGAATTAGCAGAACGTGCGTG |
| | | | GTGAGGAGATGCCAAAGGCAAGAAGAGAAGAGTATTC |
| | | | CAAACAGGAGGGATTCCAAAGAGAGAAGAGTATCCCA |
| | | | AACAACATTTGCACAAACCTGATGGGGAGAGAGAATGT |
| | | | GGGGTGGGGATGGATGATGAGACTGAAGAAGAAAGCC |
| | | | AGGTCTAGATAATCAGTGGCCTTGTACACCATGTTAAA |
| | | | GAGTGTAGACTTGATTCTGTTGTAAACAGGAAAGCAGC |
| | | | ACAATTCATATGAATATTTTAGAAGACTCCCACTGGAA |
| | | | TATGGAGAATAAAGTTGGAGATGACTAATCCTGGAAGC |
| | | | AGGGAGAACATTTTTGAGGAAGTTGCACTATTTTGGTG |
| | | | AAAATGATGATCATAAACATGAAGAATTGTAGGTGATC |
| | | | ATGACCTCCTCTCTAATTTTCCAGAAGGGTTTTGGAAGA |
| | | | TATAACATAGGAACATTGACAGGACTGACGAAAGGAG |
| | | | ATGAAATACACCATATAAATTGTCAAACACAAGGCCAG |
| | | | ATGTCTAATTATTTTGCTTATGTGTTGAAATTACAAATT |
| | | | TTTCATCAGGAAACCAAAAACTACAAAACTTAGTTTTC |
| | | | CCAAGTCCCAGAATTCTATCTGTCCAAACAATCTGTACC |
| | | | ACTCCACCTATATCCCTACCTTTGCATGTCTGTCCAACC |
| | | | TCAAAGTCCAGGTCTATACACACGGGTAAGACTAGAGC |
| | | | AGTTCAAGTTTCAGAAAATGAGAAAGAGGAACTGAGTT |
| | | | GTGCTGAACCCATACAAAATAAACACATTCTTTGTATA |
| | | | GATTCTTGGAACCTCGAGAGGAATTCACCTAACTCATA |
| | | | GGTATTTGATGGTATGAATCCATGGCTGGGCTCGGCTTT |
| | | | TAAAAAGCCTTATCTGGGATTCCTTCTATGGAACCAAGT |
| | | | TCCATCAAAGCCCATTTAAAAGCCTACATTAAAAACAA |
| | | | AATTCTTGCTGCATTGTATACAAATAATGATGTCATGAT |
| | | | CAAATAATCAGATGCCATTATCAAGTGGAATTACAAAA |
| | | | TGGTATACCCACTCCAAAAAAAAAAAAAAAGCTAAATT |
| | | | CTCAGTAGAACATTGTGACTTCATGAGCCCTCCACAGC |
| | | | CTTGGAGCTGAGGAGGGAGCACTGGTGAGCAGTAGGTT |
| | | | GAAGAGAAAACTTGGCGCTTAATAATCTATCCATGTTTT |
| | | | TTCATCTAAAAGAGCCTTCTTTTTGGATTACCTTATTCA |
| | | | ATTTCCATCAAGGAAATTGTTAGTTCCACTAACCAGAC |
| | | | AGCAGCTGGGAAGGCAGAAGCTTACTGTATGTACATGG |
| | | | TAGCTGTGGGAAGGAGGTTTCTTTCTCCAGGTCCTCACT |
| | | | GGCCATACACCAGTCCCTTGTTAGTTATGCCTGGTCATA |
| | | | GACCCCCGTTGCTATCATCTCATATTTAAGTCTTTGGCT |
| | | | TGTGAATTTATCTATTCTTTCAGCTTCAGCACTGCAGAG |
| | | | TGCTGGGACTTTGCTAACTTCCATTTCTTGCTGGCTTAG |
| | | | CACATTCCTCATAGGCCCAGCTCTTTTCTCATCTGGCCC |
| | | | TGCTGTGGAGTCACCTTGCCCCTTCAGGAGAGCCATGG |
| | | | CTTACCACTGCCTGCTAAGCCTCCACTCAGCTGCCACCA |
| | | | CACTAAATCCAAGCTTCTCTAAGATGTTGCAGACTTTAC |
| | | | AGGCAAGCATAAAAGGCTTGATCTTCCTGGACTTCCCTT |
| | | | TACTTGTCTGAATCTCACCTCCTTCAACTTTCAGTCTCA |
| | | | GAATGTAGGCATTTGTCCTCTTTGCCCTACATCTTCCTT |
| | | | CTTCTGAATCATGAAAGCCTCTCACTTCCTCTTGCTATG |
| | | | TGCTGGAGGCTTCTGTCAGGTTTTAGAATGAGTTCTCAT |
| | | | CTAGTCCTAGTAGCTTTTGATGCTTAAGTCCACCTTTTA |
| | | | AGGATACCTTTGAGATTTAGACCATGTTTTTCGCTTGAG |
| | | | AAAGCCCTAATCTCCAGACTTGCCTTTCTGTGGATTTCA |
| | | | AAGACCAACTGAGGAAGTCAAAAGCTGAATGTTGACTT |
| | | | TCTTTGAACATTTCCGCTATAACAATTCCAATTCTCCTC |
| | | | AGAGCAATATGCCTGCCTCCAACTGACCAGGAGAAAGG |
| | | | TCCAGTGCCAAAGAGAAAAACACAAAGATTAATTATTT |
| | | | CAGTTGAGCACATACTTTCAAAGTGGTTTGGGTATTCAT |
| | | | ATGAGGTTTTCTGTCAAGAGGGTGAGACTCTTCATCTAT |
| | | | CCATG |
| 575 | PGR | 3388372 | GCCTTGTAAGTAGCCATGGAATGTCAACCTGTAACTTA |
| | | | AATTATCCACAGATAGTCATGTGTTTGATGATGGGCACT |
| | | | GTGGAGATAACTGACATAGGACTGTGCCCCCCTTCTCT |
| | | | GCCACTTACTAGCTGGATGAGATTAAGCAAGTCATTTA |
| | | | ACTGCTCTGATTAAACCTGCCTTTCCCAAGTGCTTTGTA |
| | | | ATGAATAGAAATGGAAACCAAAAAAAACGTATACAGG |
| | | | CCTTCAGAAATAGTAATTGCTACTATTTTGTTTTCATTA |
| | | | AGCCATAGTTCTGGCTATAATTTTATCAAACTCACCAGC |
| | | | TATATTCTACAGTGAAAGCAGGATTCTAGAAAGTCTCA |
| | | | CTGTTTTATTTATGTCACCATGTGCTATGATATATTTGGT |
| | | | TGAATTCATTTGAAATTAGGGCTGGAAGTATTCAAGTA |
| | | | ATTTCTTCTGCTGAAAAAATACAGTGTTTTGAGTTTAGG |
| | | | GCCTGTTTTATCAAAGTTCTAAAGAGCCTATCACTCTTC |
| | | | CATTGTAGA |
| 576 | PGR | 3388373 | GTCCTATCTACTAATGTCTCCATTACTATTTAGTCATCA |
| | | | TAACCATTATCTTCATTTTACATGTCGTGTTCTTTCTGGT |
| | | | AGCTCTAAAATGACACTAAATCATAAGAAGACAGGTTA |
| | | | CATATCAGGAAATACTTGAAGGTTACTGAAATAGATTC |
| | | | TTGAGTTAATGAAAATATTTTCTGTAAAAAGGTTTGAA |
| | | | AAGCCATTTGAGTCTAAAGCATTATACCTCCATTATCAG |
| | | | TAGTTATGTGACAATTGTGTG |
| 577 | PGR | 3388374 | GCAGTAATTTGCCTTCTCCTAGAGTTTACCTGCCATTTT |
| | | | GTGCACATTTGAGTTACAGTAGCATGTTATTTTACAATT |
| | | | GTGACTCTCCTGGGAGTCTGGGAGCCATATAAAGTGGT |
| | | | CAATAGTGTTTGCTGACTGAGAGTTGAATGACATTTTCT |
| | | | CTCTGTCTTGGTATTACTGTAGATTTCGATCATTCTTTGG |
| | | | TTACATTTCTGCATATTTCTGTACCCATGACTTTATCACT |
| | | | TTCTTCTCCCATGCTTTATCTCCATCAATTATCTTCATTA |
| | | | CTTTTAAATTTTCCACCTTTGCTTCCTACTTTGTGAGATC |
| | | | TCTCCCTTTACTGACTATAACATAGAAGAATAGAAGTG |
| | | | TATTTTATGTGTCTTAAGGACAATACTTTAGATTCCTTG |
| | | | TTCTAAGTTTTTAAACTGAATGAATGGAATATTATTTCT |
| | | | CTCCCTAAGCAAAATTCCACAAAACAATTATTTCTTATG |
| | | | TTTATGTAGCCTTAAATTGTTTTGTACTGTAAACCTCAG |
| | | | CATAAAAACTTTCTTCATTTCTAATTTCATTCAACAAAT |
| | | | ATTGATTGAATACCTGGTATTAGCACAAGAAAAATGTG |
| | | | CTAATAAGCCTTATGAGAATTTGGAGCTGAAGAAAGAC |
| | | | ATATAACTCAGGAAAGTTACAGTCCAGTAGTAGGTATA |
| | | | AATTACAGTGCCTGATAAATAGGCATTTTAATATTTGTA |
| | | | CACTCAACGTATACTAGGTAGGTGCAAAACATTTACAT |
| | | | ATAATTTTACTGATACCCATGCAGCACAAAGGTACTAA |
| | | | CTTTAAATATTAAATAACACCTTTATGTGTCAGTAATTC |
| | | | ATTTGCATTAAATCTTATTGAAAAGGCTTTCAATATATT |
| | | | TTCCCCACAAATGTCATCCCAAGAAAAAAGTATTTTTA |
| | | | ACATCTCCCAAATATAATAGTTACAGGAAATCTACCTCT |
| | | | GTGAGAGTGACACCTCTCAGAATGAACTGTGTGACACA |
| | | | AGAAAATGAATGTAGGTCTATCCAAAAAAAACCCCAAG |
| | | | AAACAAAAACAATATTATTAGCCCTTTATGCTTAAGTG |
| | | | ATGGACTC |
| 578 | PGR | 3388375 | TAGGTAACTCCCTTTGTGTCAATTATATTTCCAAAAATG |
| | | | AACCTTTAAAATGGTATGCAAAATTTTGTCTATATATAT |
| | | | TTGTGTGAGGAGGAAATTCATAACTTTCCTCAGATTTTC |
| | | | AAAAGTATTTTTAATGCAAAAAATGTAGAAAGAGTTTA |
| | | | AAACCACTAAAATAGATTGATGTTCTTCAAACTAGGCA |
| | | | AAACAACTCATATGTTAAGACCATTTTCCAGATTGGAA |
| | | | ACACAAATCTCTTAGGAAGTTAATAAGTAGATTCATAT |
| | | | CATTATGCAAATAGTATTGTGGGTTTTGTAGGTTTTTAA |
| | | | AATAACCTTTTTTGGGGAGAGAATTGTCCTCTAATGAG |
| | | | GTATTGCGAGTGG |
| 579 | PGR | 3388376 | CAAACAACTTCATCTGTACTGCTTGAATACATTTATCCA |
| | | | GTCCCGGGCACTGAGTGTTGAATTTCCAGAAATGATGT |
| | | | CTGAAGTTATTGCTGCACAATTACCCAAGATATTG |
| 580 | PGR | 3388377 | ATGAGGTCAAGCTACATTAGAGAGCTCATCAAGGCAAT |
| | | | TGGTTTGAGGCAAAAAGGAGTTGTGTCGAGCTCACAGC |
| | | | GTTTCTATCAACTTA |
| 581 | PGR | 3388378 | TGGAAGGGCTACGAAGTCAAACCCA |
| 582 | PGR | 3388379 | CAGGAGTTTGTCAAGCTTCAAGTTAGCCAAGAA |
| 583 | PGR | 3388382 | TATTTTGCACCTGATCTAATACTAAATGA |
| 584 | PGR | 3388383 | CTTACATATTGATGACCAGATAACTCTCATTCAGTATTC |
| | | | TTGGATGAGCTTAATGGTGTTTGGTCTAGGATGGA |
| 585 | PGR | 3388387 | CAGCCAGTGGGCGTTCCAAATGAAAGCCAAGCCCTAAG |
| | | | CCAGAGATTCACTTTTTCACCAGGTCAAGACATACAGTT |
| | | | GATTCCACCACTGATCAACCTGTTAATGAGCATTGAAC |
| | | | CAGATGTGATCTATGCAGGACATGACAACACAAAACCT |
| | | | GACACCTCCAGTTCTTTGCTGACAAGTCTTAATCAACTA |
| | | | GGCGAGAGGCAACTTCTTTCAGTAGTCAAGTGGTCTA |
| 586 | PGR | 3388388 | ATGGAGTTTATATATATTTACATGAATTTCTTTTTTTTCT |
| | | | TCTCTG |
| 587 | PGR | 3388389 | AGACCATTGGCGTACCACCAGTTTGTGGAGGGAACTGG |
| | | | AAAAACTGGAATACACATGCCCCATCCAAAAGCAACCA |
| | | | TTGCAACTAAACTTTAACAGATTGTTGCCACCTAAGTAA |
| | | | TTCACGGATGGTCTCATAATTCTGGTCAGCATTGTCTGA |
| | | | GCCAAACAAAATGTATCTATGGGCATGATCAGATACTA |
| | | | GAGCCAGCAGATTGCAACCTCTGCTTAGATAATTGCAG |
| | | | GTATCAGCCTTCCCTTGGCTAAACAGCTACTTCATACTG |
| | | | ATAAGTAGCCCTTGCCTGGCACAAAGCAGGTGGGGCTG |
| | | | AATCCAGCCTGATATCACATCACCACAACTTTCTCTAAT |
| | | | TCTCCTCAAGGCGTCTGTGAACTACCA |
| 588 | PGR | 3388393 | TGACTGCATCGTTGATAAAATCCGCAGAAAAAACTGCC |
| | | | CAGCATGTCGCCTTAGAAAGTGCTG |
| 589 | PGR | 3388407 | CAGGCTGTCATTATGGTGTCCTTACCTGTGGGAGCTGTA |
| | | | AGGTCTTCTTTAAGAGGGCAATGGAA |
| 590 | PGR | 3388408 | AGCCAGAGCCCACAATACAGCTTCGAGTCATTACCTCA |
| | | | GAAGATTTGTTTA |
| 591 | PGR | 3388410 | TCGGCTACCAGGCCGCCGTGCTCAAGGAGGGCCTGCCG |
| | | | CAGGTCTACCCGCCCTATCTCAACTACCTGA |
| 592 | PGR | 3388411 | GCCGCCCTGCAAGGCGCCGGGCGCGAGCGGCTGCCTGC |
| | | | TCCCGCGGGACGGCCTGCCCTCCACCTCCGCCTCTGCCG |
| | | | CCGCCGCCGGGGCGGCCCCCGCGCTCTACCCTGCACTC |
| | | | GGCCTCAACGGGCTC |
| 593 | PGR | 3388412 | GTGCCTCAGTCTCGTCTGCGTCCTCCTCGGGGTCGACCC |
| | | | TGGAGTGCATCCTGTACAAAGCGGAGGGCGCG |
| 594 | PGR | 3388413 | CTGCCTCTCAATCACGCCTTATTGGCAGCCCGCACTCGG |
| | | | CAGCTGCTGGAAGACGAAAGTTACGACGGCGGGGCCG |
| | | | GGGCTGCCAGCGCCTTTGCCCCGCCGCGGAGTTCACCC |
| | | | TGTGCCTCGTCCACCCCGGTCGCTGTAGGCGACTTCCCC |
| | | | GACTGCGCGTACCCGCCCGACGCCGAGCCCAAGGACGA |
| | | | CGCGTACCCTCTCTATAGCGACTTCCAGCCGCCCGCTCT |
| | | | AAAGATAAAGG |
| 595 | PGR | 3388414 | TTGCCTGAAGTTTCGGCCATACCTATCTCCCTGGACGGG |
| | | | CTACTCTTCCCTCGGCCCTGCCAGGGACAGGACCCCTCC |
| | | | GACGAAAAGACGCAGGACCAGCAGTCGCTGTCGGACG |
| | | | TGGAGGGCGCATATTCCAGAGCTGAAGCTACAAGGGGT |
| | | | GCTGGAGGCAGCAGTTCTAGTCCCCCAGAAAAGGACAG |
| | | | CGGACTGCTGGACAGTGTCTTGGACACTCTGTTGGCGC |
| | | | CCTCAGGTCCCGGGCAGAGCCAACCCAGCCCTCCCGCC |
| | | | TGCGAGGTCACCAGCTCTTGGTGCCTGTTTGGCCCCGAA |
| | | | CTTCCCGAAGATCCACCGGCTGCCCCCGCCACCCAGCG |
| | | | GGTGTTGTCCCCGCTCATGAGCCGGTCCGGGTGCAAGG |
| | | | TTGGAGACAGCTCCGGGACGGCAGCTGCCCATAAAGTG |
| | | | CTGCCCCGGGGCCTGTCACCAGCCCGGCAGCTGCTGCT |
| | | | CCCGGCCTCTGAGAGCCCTCACTGGTCCGGGGCCCCAG |
| | | | TGAAGCCGTCTCCGCAGGCCGCTGCGGTGGAGGTTGAG |
| | | | GAGGAGGATGGCTCTGAGTCCGAGGAGTCTGCGGGTCC |
| | | | GCTTCTGAAGGGCAAACCTCGGGCTCTGGGTGGCGCGG |
| | | | CGGCTGGAGGAGGAGCCGCGGCTGTCCCGCCGGGGGC |
| | | | GGCAGCAGGAGGCGTCGCCCTGGTCCCCAAGGAAGATT |
| | | | CCCGCTTCTCAGCGCCCAGGGTCGCCCTGGTGGAGCAG |
| | | | GACGCGCCGATGGCGCCCGGGCGCTCCCCGCTGGCCAC |
| | | | CACGGTGATGGATTTCATCC |
| 596 | PGR | 3388415 | TGAAATCTACAACCCGAGGCGGCTAGTGCTCCCGCACT |
| | | | ACTGGGATCTGAGATCTTCGGAGATGACTGTCGCCCGC |
| | | | AGTACGGAGCCAGCAGAAGTCCGACCCTTCCTGGGAAT |
| | | | GGGCTGTACCGAGAGGTCCGACTAGCCCCAGGGTTTTA |
| | | | GTGAGGGGGCAGTGGAACTCAGCGAGGGACTGAGAGC |
| | | | TTCACAGCATGCACGAGTTTGATGCCAGAGAAAAAGTC |
| | | | GGGAGATAAAGGAGCCGCGTGTCACTAAATTGCC |
| 597 | MDM2 | 3421302 | CGCACCGAGGCACCGCGGCGAGCTTGGCTGCTTCTGGG |
| | | | GCCTGTGTGGCCCTGTGTGTCGGAAAGATGGAGCAAGA |
| | | | AGCCGAGCCCGAGGGGCGGCCGCGACCCCTCTGACCGA |
| | | | GATCCTGCTGCTTTCGCAGCCAGGAGCACCGTC |
| 598 | MDM2 | 3421303 | GTACGAGCGCCCAGTGCCCTGGCCCGGAGAGTGGA |
| 599 | MDM2 | 3421304 | GAGGCCCAGGGCGTCGTGCTTCCGCGCGCCCCGT |
| 600 | MDM2 | 3421305 | ACTCCAAGCGCGAAAACCCCGGATGGTGAGGAGCA |
| 601 | MDM2 | 3421306 | TTCAGTGGCGATTGGAGGGTAGACCTGTGGGCACGGAC |
| | | | GCACGCCACTTTTTCTCTG |
| 602 | MDM2 | 3421307 | TGTACCTACTGATGGTGCTGTAACCACCTC |
| 603 | MDM2 | 3421310 | ACCAAAGCCATTGCTTTTGAAGTTATTAAA |
| 604 | MDM2 | 3421311 | GTCTGTTGGTGCACAAAAAGACACTTATACTATGAAA |
| 605 | MDM2 | 3421312 | GACTTCTTGGGCATCCCTGGATCCCAGGTTAAGAACTTC |
| | | | TGCACTAGAGATACATGA |
| 606 | MDM2 | 3421313 | GTACAGTATACTGATCTTTCTGGGATAG |
| 607 | MDM2 | 3421314 | GGGCTCAAGGGATCTGCTTACCTCGGCCTCCTAA |
| 608 | MDM2 | 3421315 | TATGACTAAACGATTATATGATGAGAA |
| 609 | MDM2 | 3421316 | CTTCTAGGAGATTTGTTTGGCGTGCCAAGCTTCTCT |
| 610 | MDM2 | 3421317 | ATTGTAAAAAGCCATCTGGGCTAACATTTC |
| 611 | MDM2 | 3421321 | AGGTTAAATTGCATAAGGGTTTGTGTTAGACTGATAGC |
| | | | ATATCTACTGAGTAGCGCCCCGCCGCCCCCCGCCCACC |
| | | | ACCAAGTTTCTGATCCTTTT |
| 612 | MDM2 | 3421322 | AATCATCGGACTCAGGTACATCTGTGA |
| 613 | MDM2 | 3421323 | GTGAGAACAGGTGTCACCTTGAAGGTGGGAG |
| 614 | MDM2 | 3421325 | TCACATTTGGTTTCTAGACCATCTACCTCATCTAGAAGG |
| | | | AGAGCAATTAGTGAGACA |
| 615 | MDM2 | 3421326 | ATATTTATTTGACGCATTCACACAGCTTTTTGATATTCTT |
| | | | TCTCTAATGAAATTAGTGCTTTTAGACTTAAT |
| 616 | MDM2 | 3421328 | TCAGATGAATTATCTGGTGAACGACAAAGAAAACGCCA |
| | | | CAAATCTGATAGT |
| 617 | MDM2 | 3421329 | AAAGCCTGGCTCTGTGTGTAATAAGGGAGATATGTTGT |
| | | | GAAAGAAGCAGTAGCAGTGAATCTACAGGGACGCCA |
| 618 | MDM2 | 3421334 | TTCAGTTTCAGATCAGTTTAGTGTAGAATTTGAAGTTGA |
| | | | ATCTCTCGACTCAGAAGATTATAGCCTTAGTGAAGA |
| 619 | MDM2 | 3421336 | TGCAATGAAATGAATCCCCCCCTTCCATCACATTGCAAC |
| | | | AGATGTTGGGCCCTTCGTGAGAATTGGCTTCCTGAAGA |
| 620 | MDM2 | 3421337 | CTATAGTGAATGATTCCAGAGAGTCATGTGTTGAGGAA |
| | | | AATGATGATAAAATTACACAAGCTTCACAAT |
| 621 | MDM2 | 3421338 | TCTCAGCCATCAACTTCTAGTAGCA |
| 622 | MDM2 | 3421339 | AAGAGAGTGTGGAATCTAGTTTGCCCCTTAATGCCATT |
| | | | GAA |
| 623 | MDM2 | 3421340 | TCAAGGTCGACCTAAAAATGGTTGCATTGTCC |
| 624 | MDM2 | 3421341 | ATATTTCTAACTATATAACCCTAGGA |
| 625 | MDM2 | 3421342 | ACCGCGTCCGGCCTAAATGTCACTTAGTACCTTTGATAT |
| | | | AAAGAGAAAATGTGTGAAAGATTTAGTTTTTTGTTTTTT |
| | | | TGTTTGTTTGTTTGTTTGTTTGTTTTGAGATGAGTCTCTC |
| | | | TGTCGCCCAGGCTGGAGTGCAGTGTCATGATCTAGCAG |
| | | | TCTCCGCTTCCCGGGTTCAAGCCATTCTCCTGGCTCAGC |
| | | | CTCTGGAGCAGCTGGGATTACAGGCATGCACCACCATG |
| | | | CCCAGCTAATTTTTGTATTTTTAGTAGAGATAGGGTTTC |
| | | | ACCATGTTGGCCAGGCTGGTCACGAACTCCTGACCTCA |
| | | | AGTGAGGTCACCCGCCTCGGCCTCCCGAAGTGCTGGGA |
| | | | TTGCAGATGTGAGCCACCATGTCCAGCCAAGAATTAGT |
| | | | ATTTAAATTTTAGATACTCTTTTTTTTTTTTTTTTTTTTTT |
| | | | TTTTTGAGACAGAGTCTTGCTCCATCACCCATGCTAGAG |
| | | | TGCAGTGGAGTGATCTCGGCTCACTGCAACTTCCGCCTT |
| | | | CTGGGTTCAAGCTATTCTCCTGCCTCAGCCTTCCAAGTA |
| | | | ACTGGGATTACAGGCATGTACCACCATACCAGCTGATT |
| | | | TTTTTGTATTTTTAGTAAAGACAGGGTTTCACCATGTTA |
| | | | GCCAGGCTGATCTTGAACTCCTAAACTCAAGTGATCTA |
| | | | CTCACCTCAGCCTCCCAAAATGCTGGGATTACAGATGT |
| | | | GAGGCACCTGGCCTCAGATTTTTGA |
| 626 | MDM2 | 3421343 | TGGAGGCCCATCCGAGCTCAGCACTGA |
| 627 | MDM2 | 3421344 | ACAAGCCTGTCAAATATCTGCAAGAACTATGGAATAAA |
| | | | ACTACTGATGCAGTGAAGACAGTTGAAAAGATCAAACA |
| | | | AATGCCAAGCTATATTTATAATGAACAAATTCAAGAAA |
| | | | AAGGACTACGGAAAGTTCAGGACATCAAAGAAGTCAG |
| | | | GCAAAACTCATCTTGACCCCTGTTGCAGGCAAAGGAAC |
| | | | GCAGCTGGAAGAAAAGATGATATAACAGTTAACAGGA |
| | | | TGCAGACATGGCAGAGGTTTCCTAAAAATCTCATTATCT |
| | | | ATAACCATTTCTATATTTACATTTGAAAATCTCCTTTGG |
| | | | AGACTTAGAACCTCTAAATTATTGACTTATTTTTTATAT |
| | | | AAGGTCACTCCGATGAAAGGTGATTACA |
| 628 | MDM2 | 3421345 | TGAGAGCCGAATAAGGTTTGCCTGAAATAACTGACACT |
| | | | ATATAATTTCTGCTTTGGCAAATACTAAGTTCTAACTTG |
| | | | TCATTCCTGGTAGAACAAGCTTTATTTTTCGAGCCTAGC |
| | | | AATGATCTAGAAGCAGATGTTATCTCAGTGCCTTTTG |
| 629 | MDM2 | 3421346 | CTGATGGGTGTGCTAATTACACTGATTTGATCAATACCC |
| | | | ATTGTATGTGAAACAGTACATACACCATATTTACAATTA |
| | | | TGTATTTAACATTTAAAATTTCTAATATAAGTATCTCTC |
| | | | AAACTGTGGATTAACTTCTTGATTTATATTTAAATATGA |
| | | | ATCTTAAGCAAAACAGTGAAAATAACCATCTTGATTTA |
| | | | GTGTTTTTCTCCCATATGTGAATTGTATATAC |
| 630 | MDM2 | 3421347 | GCTTAGAATAGGACTGAGGTAATTCTGCACAGCAACTT |
| | | | TACTAATGGTA |
| 631 | MDM2 | 3421348 | GTCCTCCAAGCATTATTTGGAGTTGATAATACTTCAGCT |
| | | | ACAACCAAGCAGAATCTCTTTTTTTTGGA |
| 632 | MDM2 | 3421349 | GATAATACTTCAGCTTCAATTTGGAGTTGATAATATTTC |
| | | | AGCTAGAACCTAGTAGAATC |
| 633 | MDM2 | 3421350 | CACTTGATATATGGAGGCAGTGACAGCTATTTTTACAA |
| | | | AATTTAAATCTGCAAATGGATTCAACATGTTTATGGGTT |
| | | | ATTAAAATTGTCTGATTTCTTAGGTTCTTTATAGTACAC |
| | | | GTGTTGA |
| 634 | MDM2 | 3421351 | TGGGAGCCTCCAATGAGAGCAACTTGAGAGAATGATGT |
| | | | TGCAAGTTAGTAGGAGTAAGAAATGCTGTGTTCTCCCT |
| | | | GTCTTCTCTTAGGTCACATGGCAGCCTGGCCTAAGTGAT |
| | | | CGTGAATGGTCTATAAGGGAGGTAGCTGGGACAGGGA |
| | | | GGGGAGTTTGGGCTAGCCACCGTACCACTTGTCAGCGT |
| | | | GAAAAGTAAGATTGTAATTGCCTGTTTAGTTTTCTGCCT |
| | | | CATCTTTGAAAGTTCCACCAAGCTGGGAACCTCTTGATT |
| | | | GTGAGGCACAAATGTAAGTACATCAGAAAAAAACAAA |
| | | | AAAACTGGCTTTAAAGCAGGAGCTTGTGGGCCCCTAAG |
| | | | CCAGACGGGGACTAGCTTTTGGCATTATATAATTAAGA |
| | | | TTTTTTAAATCCTTAATAAGGGTTTTATTTTATTTTTATT |
| | | | TATTTTTTGAGACGGAGTCTTGCTCTGTGGCTCAGGCTG |
| | | | GAGTACAGTGGTGCAATCTTGGCTCACTGCAACCTCTG |
| | | | CCTCCTGGCTGTGTTCAAGTGGTTCT |
| 635 | MDM2 | 3421359 | GAAACATTAAGAATACCATATGAGTAAATTAAACACTT |
| | | | TGGCTCTTTTCGGAAAAAAACAGATGAGCTCTTATATTT |
| | | | TAAAGTTTGGTTTTGACAGAAAAAAATTCCTAGATTTTT |
| | | | TGGTTAATAAAAACTTTATTAGATAGATTAAATTGTGAT |
| | | | G |
| 636 | MDM2 | 3421361 | AGGGAACTTCTGCTTAAGAGGCTTCTATGTAATGAAAT |
| | | | TCTCTTGAAAACAGAGAAACTATTTCCTGTTTATTTTCT |
| | | | AAATTGAGACGTCACTTTTTAAAAATTGGTACCTGTAAT |
| | | | TTAGCCATTTCCTACTCAGCAATGTCTCATTTAAACTAT |
| | | | TATTTGTTTAGCGTGTTTCAAAGAGCAGATGTAAGCTTG |
| | | | AGCCCATCCTCTGTCCTATGACTAAGTCGATATTAGCAG |
| | | | GGGTTAGGACTGTTAGTTTTCCAGTTCCTACTGGAGGCA |
| | | | AATTCTT |
| 637 | KRT5 | 3455207 | CCTTTTCTGGAGAGTAGTCTAGACCAAGCCAATTGCAG |
| | | | AACCACATTCTTTGGTTCCCAGGAGAGCCCCATTCCCAG |
| | | | CCCCTGGTCTCCCGTGCCGCAGTTCTATATTCTGCTTCA |
| | | | AATCAGCCTTCAGGTTTCCCACAGCATGGCCCCTGCTGA |
| | | | CACGAGAACCCAAAGTT |
| 638 | KRT5 | 3455208 | TCTTGCCGGAGGTAGCAGTGGAAGCTACTACTCCAGCA |
| | | | GCAGTGGGGGTGTCGGCCTAGGTGGTGGGCTCAGTGTG |
| | | | GGGGGCTCTGGCTTCAGTGCAAGCAGTGGCCGAGGGCT |
| | | | GGGGGTGGGCTTTGGCAGTGGCGGGGGTAGCAGCTCCA |
| | | | GCGTCAAATTTGTCTCCAC |
| 639 | KRT5 | 3455211 | ACTCAGTGGAGAAGGAGTTGGACCAGTCAACATCT |
| 640 | KRT5 | 3455212 | AGGTCAATCCCTCTTCATTGGAAAATCCCTCTGGAGAGT |
| | | | TCTCCCTTCCTTTAACTTAAGCAGCTTTTGGGTGTACAG |
| | | | ACTCCTGGCTTATGGAATGAACTCGAATCATGAGGATG |
| | | | GGAGTTAGCCACATAGACTAATGCTGTCTTTTTGGGAG |
| | | | CTGTTAACCCTTAATTCA |
| 641 | KRT5 | 3455214 | CAAGATGGCCTTCAGCTGATAAAGCGAAGCTGCTCTAC |
| | | | TGTGGGGTGTACAACACACATACATGAGATCAGTGACT |
| | | | TGTGCGTGATAATGACACATCATCAACACTATTTCAGTC |
| | | | TGACTCATGGCCATATAGCTGACCTCAACTCACTTTTCT |
| | | | GGTCTCTTTTCCCCCACCGGTGTTCCTGGGCACTGGCTG |
| | | | TCCTCCAAGCACCTGAGCAACTCAGCAATCTTCTTGACA |
| | | | CTTGTGCCTTTTCTGCTTTTGCTCACGTCCTTTGCTCAAC |
| | | | CTCAATATCCATGTCATG |
| 642 | KRT5 | 3455215 | TGCGCCAATCTGCAGAACGCCATTGCGGATGCCGAGC |
| 643 | KRT5 | 3455216 | CTTTTGCTAAACACACGCAGCTAGATCCAGTACCAGTG |
| | | | TTTCAGTGTCCTGCCACCCACGATGTACTGGTTTCTCTC |
| | | | TGGGATTCATGATAGTTTGGTTTGTCTGACCCAGAAACT |
| | | | CAG |
| 644 | KRT5 | 3455217 | AGCTGGCCGGCATGGCGATGACCTCCGCAACACCAAGC |
| | | | ATGAGATCTCTGAGATGAACCGGATGATCCAGAGGCTG |
| | | | AGAGCCGAGATTGACAATGTCAAGA |
| 645 | KRT5 | 3455219 | CAGATGCAGACGCATGTCTCTGACACCTCAGTGGTCCT |
| | | | CTCCATGGACAACAACCGCAACCTGGACCTGGATAGCA |
| | | | TCATCGCTGAGGTCAAGGCCCAGTATGAGGAGATTGCC |
| | | | AACCGCAGCCGGACAGAAGCCGAGTCCTGGTATCAGAC |
| | | | CAAG |
| 646 | KRT5 | 3455220 | CAGAACCAGATGACCGACTCCAAATCTCCCTG |
| 647 | KRT5 | 3455222 | GGCCAAGGTTGATGCACTGATGGATGAGATTAACT |
| 648 | KRT5 | 3455223 | GCAGCCTGCAGCTATGCTCTCTAAGCGTGGAGCTCACTT |
| | | | GAGTAGGGTGACGGTGTG |
| 649 | KRT5 | 3455224 | TGAAATCAACAAGCGTACCACTGCTGAGAATGAG |
| 650 | KRT5 | 3455225 | AAAAGAACACTAGAGAAATTGACTAG |
| 651 | KRT5 | 3455228 | GTGGACACGTTCTGAATTAGACTGGCAGCTGGGAAG |
| 652 | KRT5 | 3455229 | CAACCTGCAAATCGACCCCAGCATCCAGAGGGTGAGGA |
| | | | CCGAGGAGCGCGAGCAGATCAAGA |
| 653 | KRT5 | 3455230 | GAGGTGGTGCCGGTAGTGGATTTGGTTTCGGCGGTGGA |
| | | | GCTGGTGGT |
| 654 | KRT5 | 3455231 | GGCAGCTTCAGGAACCGGTTTGGTGCTGGTGC |
| 655 | KRT5 | 3455232 | GGGCTCCAAGAGGATATCCATCAGCACTAGTG |
| 656 | KRT5 | 3455233 | TGTGGAGTGGGTGGCTATGGCAGCCGGAGCCTCTACAA |
| | | | CCTGGG |
| 657 | KRT5 | 3455234 | TGGTGGCTTCGGCAGGGTCAGCCTTGCGG |
| 658 | KRT5 | 3455235 | TCAAGTGTGTCCTTCCGGAGCGGGGGCAGTCGTAGCTT |
| | | | CAGCACCGCCTCTGCCATCACCCCGTCTGTCTCCCGCAC |
| | | | CAG |
| 659 | KRT5 | 3455236 | ACAGCTCGACAGCTCTCTCGCCCAGCCCAGTTCTGGAA |
| | | | GGGATAAAAAGGGGGCATCACCGTTCCTGGGTAACAGA |
| | | | GCCACCTTCTGCGTCCTGCTGAGCTCTGTTCTCTCCAGC |
| | | | ACCTCCCAACCCACTAGTGCCT |
| 660 | KRT5 | 3455237 | CCCAGCCTCTATGGTGAAGACATACTTGCTAGCAGCGT |
| | | | CACCAACTTGCTGCCAAGAGATCAGTGCTGCAAGGCAA |
| | | | GGTTATTTCTAACTGAGCAGAGCCTG |
| 661 | FOXA1 | 3561726 | CAGGTCTGTGGCAATACTCTTAACCATAAGAATTGAAA |
| | | | TGGTGAAGAAACAAGTATACACTAGAGGCTCTTAAAAG |
| | | | TATTGAAAGACAATACTGCTGTTATATAGCAAGACATA |
| | | | AACAGATTATAAACATCAGAGCCATTTGCTTCTCAGTTT |
| | | | ACATTTCTGATACATGCAGATAGCAGATGTCTTTAAATG |
| | | | AAATACATGTATATTGTGTATGGACTTAATTATGCACAT |
| | | | GCTCAGATGTGTAGACATCCTCCGTATATTTACATAACA |
| | | | TATAGAGGTAATAGATAGGTGATATACATGATACATTC |
| | | | TCAAGAGTTGCTTGACCGAAAGTTACAAGGACCCCAAC |
| | | | CCCTTTGTCCTCTCTACCCACAGATGGCCCTGGGAATCA |
| | | | ATTCCTCAGGAATTGCCCTCAAGAACTCTGCTTCTTGCT |
| | | | TTGCAGAGTGCCATGGTCATGTCATTCTGAGGTCACATA |
| | | | ACACATAAAATTAGTTTCTATGAGTGTATACCATTTAAA |
| | | | GAATTTTTTTTTCAGTAAAAGGGAATATTACAATGTTGG |
| | | | AGGAGAGATAAGTTATAGGGAGCTGGATTTCAAAACGT |
| | | | GGTCCAAGATTCAAAAATCCTATTGATAGTGGCCATTTT |
| | | | AATCATTGCCATCGTGTGCTTGTTTCATCCAGTGTTA |
| 662 | FOXA1 | 3561727 | GTGTGTATTCCAGACCCGTCCTAAACACTTCCTAG |
| 663 | FOXA1 | 3561728 | TCGGAGCAGCAGCATAAGCTGGACTTCAAGGCATACGA |
| | | | ACAGGCACTGCAATACTCGCCTTACGGCTCTACGTTGCC |
| | | | CGCCAGCCTGCCTCTAGGCAGCGCCTCGGTGACCACCA |
| | | | GGA |
| 664 | FOXA1 | 3561729 | GGGCGCCTCGGAGTTGAAGACTCCAGCCTCCTCAACTG |
| | | | CGCCCCCCATAA |
| 665 | FOXA1 | 3561730 | GGCGGCCCTGAGAGCCGCAAGGACCCCTCTGGCGCCTC |
| | | | TAACCCCAGCGCCGACTCGCCCCTCCATCGGGGTGTGC |
| | | | ACGGGAAGACCGGCCAGCTA |
| 666 | FOXA1 | 3561731 | AGGCGCCCAGCAAGATGCTCACGCTGAGCGAGATCTAC |
| | | | CAGTGGATCATGGACCTCTTCCCCTATTACCGGCAGAA |
| | | | CCAGCAGCGCTGGCAGAACTCCATCCGCCACTCGCTGT |
| | | | CCTTCAATGACTGCTTCGTCAAGGTGGCACGCTCCCCGG |
| | | | ACAAGCCGGGCAAGGGCTCCTACTGGACGCTGCACCCG |
| | | | GACTCCGGCAACATG |
| 667 | FOXA1 | 3561732 | TACGCGCCGTCCAACCTGGGCCGCAGCCGCGCGGGCGG |
| | | | CGGCGGCGACGCCAAGACGTTCAAGCGCAG |
| 668 | FOXA1 | 3561733 | TCCGTCCCGGTCAGCAACATGAACTCAGGCCTGGGCTC |
| | | | CATGAACTCCATGAACACCTACATGACCATGAACACCA |
| | | | TGACTACGAGCGGCAACATGACCCCGGCGTCCTTCAAC |
| | | | ATGTCCTATGCCAACCCGGGCCTAGGGGCCGGCCTGAG |
| | | | TCCCGGCGCAGTAGCCGGCATGCCGGGGGGCTCGGCGG |
| | | | GCGCCATGAACAGCATGACT |
| 669 | FOXA1 | 3561736 | TGGAAGGGCATGAAACCAGCGACTGGAACAGCTACTAC |
| | | | GCAGACACGCAGGAG |
| 670 | FOXA1 | 3561738 | CTTTGTGCGGCGGACAAATGGGGAGAG |
| 671 | FOXA1 | 3561741 | TCATAAAGATATAAACCGGTGCTGTGACTCACCTGCTCT |
| | | | TAGCCGCAG |
| 672 | ORC6 | 3658927 | GGGGTCGGAGCTGATCGGGCGCCTAGCCCCGCGCCTGG |
| | | | GCCTCGCCGAGCCCGACATGCTGAG |
| 673 | ORC6 | 3658928 | GTGAGTTCGGCCGCGCAAGACCAGGGCTGGGCTTCCGC |
| | | | CTCGCGGCCCTGGGC |
| 674 | ORC6 | 3658931 | CTCTCACCAGAGGATATGACCTTCATTCCCAGCCCCAG |
| | | | ATAAACGAGCCACAGGAGTTAGGCTTAGTGTGAAGCTA |
| | | | ACCAGGCTGTATT |
| 675 | ORC6 | 3658932 | AAGCAGAGGAGTACTTGCGCCTGTCCCGGGTGAAGTGT |
| | | | GTCGGCCTCTCCGCACGCACCACGGAGACCAGCAGTG |
| 676 | ORC6 | 3658934 | GATCTTGACTTATCCAGGCCACTTTTCAC |
| 677 | ORC6 | 3658935 | GCCAAGTGACTATATTCCCAGTTTATCCCATAATGTAGC |
| | | | TAACAACTTGGAACTAGTGTTGCCAGAATTCCACTAGC |
| | | | AAATAGCAGCTGTATATATATGCTGGGAATTCTGATTTC |
| | | | AGTCTGCCTTTTGTAAGAGATGATATC |
| 678 | ORC6 | 3658937 | AAGTCATGAAGCACTACAGCAAATGTCTTTTATGTGCC |
| | | | CCTTTTGTTATAAAATAGATCCCATGTGCATTTTAACTC |
| | | | TCAGTCCAATAAACAACTAAACAACTTAGCATAGATAA |
| | | | TAACATGTTTGGAATGAAGGAAAAAAACTAGACAGAG |
| | | | GCTCTGGAAGCATGGTCAAAAAGAAAATAAGTTGATTA |
| | | | TCTGGTTGCCCAGAGAAGAAAACTGTACAGGTCTTGAG |
| | | | AAAAGC |
| 679 | ORC6 | 3658938 | GATTCTAAAGCTGAAAGTGGATAAAAACAA |
| 680 | ORC6 | 3658939 | AAAAAGCTATATTTGATCGACTGTGTAAACAACTAGAG |
| | | | AAGATTGGACAGCAGGTCGACA |
| 681 | ORC6 | 3658940 | TTATTTGTGATCCAAAACATGCCCAAATACTGAAATTG |
| | | | AG |
| 682 | ORC6 | 3658941 | ACCTGGAGATGTAGCTACTCCACCACGGAAGAGAAAGA |
| | | | AGATAGTGGTTGAAGCCC |
| 683 | ORC6 | 3658943 | AAGGTAGAGGAGATGCCACATAAACCACAGAAAGATG |
| | | | AAGATCTGACACA |
| 684 | ORC6 | 3658944 | TTGGAAAATGCTGCCAGTGCTCAAAAGGCTACAGCAGA |
| | | | GTG |
| 685 | ORC6 | 3658945 | TCTCCAGGAAGACTTGACGGCTTTGGGATTTTGTTTAAA |
| | | | CTTTTATAATAAGGATCCTAAGACTGTTGCCTTTAAATA |
| | | | GCAAAGCAGCCTACCTGGAGGCTAAGTCTGGGCAGTGG |
| | | | GCTGGCCCCTGGTGTGAGCATTAGACCAGCCACAGTGC |
| | | | CTGATTGGTATAGCCTTATGTGCTTTCCTACAAAATGGA |
| | | | ATTGGAGGCCGGGCGCAGTGGCTCACGCCTGTAATCCC |
| | | | AGCACTTTGGGAGGCCAAGGTGGGTGGATCACCTGAGG |
| | | | TCAGGAGCTCGAGACCAGCCTGGCCAACATGGTGAAAC |
| | | | CCCATCTCTACTAAAAATACAAAAATTAGCCAGGTGTG |
| | | | ATGGTGCATGCCTGTAATCCCAGCTCCTCAGTAGGCTG |
| | | | AGACAGGAGCATCACTTGAACGTGGGAGGCAGAGGTT |
| | | | GCAGTGAGCCGAGATTGCACCACCGCACTCCAGCCTGG |
| | | | GTGACAGAGCGAGACTTATCTCATAAATAAATAGATAG |
| | | | ATACTCCAGCCTGG |
| 686 | ORC6 | 3658946 | TGGAGCCATTTTGCTTTAAGTGAATGGCAGTCCCTTGTC |
| | | | TTATTCAGAATATAAAATTCAGTCTGAATGGCATCTTAC |
| | | | AGATTTTACTTCAATTTTTGTGTACGGTATTTTTTATTTG |
| | | | ACTAAATCAATATATTGTACAGCCTAAGTTAATAA |
| 687 | CDH3 | 3666367 | CGGAGCCTCCGTTTTCAGTCGACTTCAGATGTGTCTCCA |
| | | | CTTTTTTCCGCTGTAGCCGCAAGGCAAGGAAACATTTCT |
| | | | CTTCCCGTACTGAGGAGGCTGAGGAGTGCACTGGGTGT |
| | | | TCTTTTCTCCTCTAACCCAGAACTGCGAGACAGAGGCTG |
| | | | AGTCCCTGTA |
| 688 | CDH3 | 3666368 | ATGGGGCTCCCTCGTGGACCTCTCGCGTCTCTCCTC |
| 689 | CDH3 | 3666369 | GCTGGCTGCAGTGCGCGGCCTCCGAGCCGTGCCGGGCG |
| | | | GTCTTCAGGGAGGCTGAAGTGACCTTGGAGGC |
| 690 | CDH3 | 3666376 | AAGAGCCAGCTCTGTTTAGCACTGATAATGATGACTTC |
| | | | ACTGTGCGGAATGGCGAGACAGTCC |
| 691 | CDH3 | 3666380 | GATCTTCCCATCCAAACGTATCTTACGAAGACACA |
| 692 | CDH3 | 3666381 | TGGGTGGTTGCTCCAATATCTGTCC |
| 693 | CDH3 | 3666382 | TTTCTACAGCATCACGGGGCCGGGGGCAGACAGCCCCC |
| | | | CTGAGGGTGTCTTCGCTGTAGAGAAGGAGACAGGCTGG |
| | | | TTGTTGTTGAATAAGCCACTGGACCGGGAGGA |
| 694 | CDH3 | 3666384 | CTCAGTGGAGGACCCCATGAACATCTCCATCATCGTGA |
| | | | CCGACCAGAATGACCACAAGCCCAAGTTTACCCAGGAC |
| | | | ACCTTCCGAGGGAGTGTCTTAGAGGGAGTCCTACCA |
| 695 | CDH3 | 3666385 | TGACAGCCACGGATGAGGATGATGCCATCTACACCTAC |
| | | | AATGGGGTGGTTGCTTACTCCATCCATAGCCAAGAACC |
| | | | AAAGGACCCACACGACCTCATGTTCACCATTCACCGGA |
| | | | GCACAGGCACCATCAG |
| 696 | CDH3 | 3666387 | AGGCCCATGTGCCTGAGAATGCAGTGGGCCATGAGGTG |
| | | | CAGAGGCTGACGGTCACTGATCTGGACGCCCCCAACTC |
| | | | ACCAGCGTGGCGTGCCACCTACCTTATCATGGGCGGTG |
| | | | ACGACGGGGACCATTTTACCATC |
| 697 | CDH3 | 3666388 | CCAGCACACCCTGTACGTTGAAGTGACCAACGAGGCCC |
| | | | CTTTTGTGCTGAAG |
| 698 | CDH3 | 3666389 | ACAGCCACCATAGTGGTCCACGTGGAGGATGTGAATGA |
| | | | GGCACCTGTGTTTGTCCCACCCTCCAAAGTCGTTGAGGT |
| | | | CCAGGA |
| 699 | CDH3 | 3666390 | GCTGTGGGCACCCTCGACCGTGAGGAT |
| 700 | CDH3 | 3666394 | ACGGGAACCCTTCTGCTAACACTGATTGATGTCAATGA |
| | | | CCATGGCCCAGTCCCTGAGCCCCGTCAGATCACCATCT |
| | | | GCAACCAAAGCCCTGTGCGCCAGGTGCTGAACATCACG |
| | | | GACAAGGACCTGTCTCCCCACACCTCCCCTTTCCAGGCC |
| | | | CAGCTCACAGATGACTCAGACATCTACTGGACGGC |
| 701 | CDH3 | 3666395 | GTGGTCTTGTCCCTGAAGAAGTTCCTGAAGCAGGATAC |
| | | | ATATGACGTGCACCTTTCTCTGTCTGACCATGGCAACAA |
| | | | AGAGCAGCTGACGGTGATCAGGGCCACT |
| 702 | CDH3 | 3666396 | GTGTGCGACTGCCATGGCCATGTCGAAACCTGCCCTGG |
| 703 | CDH3 | 3666397 | CTCCCAGAAGATGACACCCGTGACAACGTCTTCTACTA |
| | | | TGGCGAAGAGGGG |
| 704 | CDH3 | 3666398 | CACCCAGCTCCACCGAGGTCTGGAGGCCAGGCCGGAGG |
| | | | TGGTTCTCCGCAATGACGTGGCACCAACCATCATCCCG |
| | | | ACACCCATGTACCGTCCTCGGCCAGCCAACCCAGATGA |
| | | | AATC |
| 705 | CDH3 | 3666400 | GCAAGAGGCAGGACCCGCCGCTCCTAACTACCTGTTCT |
| | | | CTG |
| 706 | CDH3 | 3666401 | ACCTGAAGGCGGCTAACACAGACCCCACAGCCCCGCCC |
| | | | TACGACACCCTCTTGGTGTTCGACTATGAGGGCAGCGG |
| | | | CTCCGACGCCGCGTCCCTGAGCTCCCTCACCTCCTCCGC |
| | | | CTCCGACCAAGACCAAGATTACGATTATCTGAACGAGT |
| | | | GGGGC |
| 707 | CDH3 | 3666402 | TCTGACGTTAGAGTGGTGGCTTCCTTAGCCTTTCAGGAT |
| | | | GGAGGAATGTGGGCAGTTTGACTTCAGCACTGAAAACC |
| | | | TCTCCACCTGGGCCAGGGTTGCCTCAGAGGCCAAGTTT |
| | | | CCAGAAGCCTCTTACCTGCCGTAAAATGCTCAACCCTGT |
| | | | GTCCTGGGCCTGGGCCTGCTGTGACTGACCTACAGTGG |
| | | | ACTTTCTCTCTGGAATGGAACCTTCTTAGGCCTCCTGGT |
| | | | GCAACTTAATTTTTTTTTTTAATGCTATCTTCAAAACGTT |
| | | | AGAGAAAGTTCTTCAAAAGTGCAGCCCAGAGCTGCTGG |
| | | | GCCCACTGGCCGTCCTGCATTTCTGGTTTCCAGACCCCA |
| | | | ATGCCTCCCATTCGGATGGATCTCTGCGTTTTTATAC |
| 708 | CDH3 | 3666404 | AGAGAACCTACCCAAGATGTCAGTGAAATTGGAACATT |
| | | | CCTGACAATACCAGGGCATAAATGCAGGAATCAGGAAT |
| | | | AGGCAGCAGTGATAGAACAATTCTGTTTGTGCCCTTGTT |
| | | | AACGTGAAGTTCAA |
| 709 | ERBB2 | 3720403 | TTCCCGGATTTTTGTGGGCGCCTGCCCCGCCCCTCGTCC |
| | | | CCCTGCTGTGTCCATATATCGAGGCGATAGGGTTAAGG |
| | | | GAAGGCGGACGCCTGATGGGTTAATGAGCAAACTGAA |
| | | | GTGTTTTCCATGATCTTTT |
| 710 | ERBB2 | 3720404 | CGCAATTGAAGTACCACCTCCCGAGGGTGATTGCTTCC |
| | | | CCATGCGGGGTAGAACCTTTGC |
| 711 | ERBB2 | 3720406 | CCAGTGGTCTATACCTCCAGCAGCAAGTCGAGTGAGCA |
| | | | AGTGATGTCCTGAAAGGCCCAGTGGATCAGTGGAATGA |
| | | | AGCGGGCAGGAAGACTTAGTGCTCCTGAAACAAGGAAT |
| | | | CCAGAATCCAGGAGAAGGATGGCTCAGTGGGGCTTTCA |
| | | | AGGGACAAGTATGGGGGTTGAAGGGGTCACTGTCCCTA |
| | | | TACC |
| 712 | ERBB2 | 3720407 | GGCAAAGCAAAGCTATATTCAAGACCACATGCAAAGCT |
| | | | ACTCCCTGAGCAAAGAGTCACAGATAAAACGGGGGCA |
| | | | CCAGTAGAATGGCCAGGACAAACGCAGTGCAGCA |
| 713 | ERBB2 | 3720408 | GATGAGAGTGACATGTACTGTTGTGGACATGC |
| 714 | ERBB2 | 3720410 | TGCCCCGGGGGTCCTGGAAGCCACA |
| 715 | ERBB2 | 3720411 | AGAATGAAGTTGTGAAGCTGAGATTCCCCTCCATTGGG |
| | | | ACCGGAGAAACCAGGGGAGCCCCCCGGGCAGCCGCGC |
| | | | GCCCCTTCCCACGGGGCCCTTTACTGCGCCGCG |
| 716 | ERBB2 | 3720412 | CAGCCGGAGCCATGGGGCCGGAGCCG |
| 717 | ERBB2 | 3720413 | GAGCTGGCGGCCTTGTGCCGCTGGGGGCTCCTCCTCG |
| 718 | ERBB2 | 3720417 | TGTGCACCGGCACAGACATGAAGCTGCGGCTCCCTGCC |
| | | | AGTCCCGAGACCCACCTGGACATGCTCCGCCACCTCTA |
| | | | CCAGGGCTGCCAGGTGGTGCAGGGAAACCTGGAACTCA |
| | | | CCTACCTGCCCACCAATGCCAGCCTGT |
| 719 | ERBB2 | 3720419 | TTGGGAGCAGTTGTGAAGCTCAGAAGAGAAATGTCTGT |
| | | | GAAAAGGTTATGAACAGGAGGGAGAGTGGAAACCAAC |
| | | | CTGCTGGATCGTGTCCACAGACCCTGGAATGGGGCCAC |
| | | | ATGCTTGGTTTGTCAAATTGCAGACGCCGGCCGGGT |
| 720 | ERBB2 | 3720420 | CAGGGCTACGTGCTCATCGCTCACAACCAAGTGAGGCA |
| | | | GGTCCCACTGCAGAGGCTGCGGATTGTGCGAGGCACCC |
| | | | AGCTCTTTGAGGACAACTATGCCCTGGCCGTGCTAGAC |
| | | | AATGGAGACCCGCTGAACA |
| 721 | ERBB2 | 3720421 | AGGGGAAAGGGTCCTCTGATCATTGCTCACCC |
| 722 | ERBB2 | 3720422 | GGTCTTGATCCAGCGGAACCCCCAGCTCTGCTACCAGG |
| | | | ACACGATTTTGTGGAAGGACATCTTCCACAAGAACAAC |
| | | | CAGCTGGCTCTCACACTGATAGACACCAACCGCTCTCG |
| | | | GGCC |
| 723 | ERBB2 | 3720425 | GCCACTGCCCACTGACTGCTGCCATGAGCAGTGTGCTG |
| | | | CCGGCTGCACGGGC |
| 724 | ERBB2 | 3720427 | TGGTCACCTACAACACAGACACGTTTGAGTCCATGCCC |
| | | | AATCCCGAGGGCCGGTATACATTCGGCGCCAGCTGT |
| 725 | ERBB2 | 3720428 | ACCTTTCTACGGACGTGGGATCCTGCACCCTCGTCTGCC |
| | | | CCCTGCACAACCAAGAGGTGACAGCAGAGGATGGAAC |
| | | | ACAGCGGTGTGAGAAGTGCAGCAAGCCCTGT |
| 726 | ERBB2 | 3720429 | TGCAGTTCCTGTCCCTCTGCGCATGCAGCCTGGCCCAGC |
| | | | CCACCCTGTCCTATCCTTC |
| 727 | ERBB2 | 3720430 | TACAAGTGTCCCTATATCCCCTGTCAGTGTGGGGAGGG |
| | | | GCCCGGACCCTGATGCTCATGTGGC |
| 728 | ERBB2 | 3720431 | GGCATGGAGCACTTGCGAGAGGTGAGGGCAGTTACCAG |
| | | | TGCCAATATCCAGGAGTTTGCTGGCTGCAAGAAGATCT |
| | | | TTGGGA |
| 729 | ERBB2 | 3720432 | AACACTGCCCCGCTCCAGCCAGAGCAGCTCCAAGTGTT |
| | | | TGAGACTCTGGAAGAGATC |
| 730 | ERBB2 | 3720434 | GGCGCCTACTCGCTGACCCTGCAAGGGCTGGGCATCAG |
| | | | CTGGCTGGGGCTGCGCTCACTGAGGGAACTGGGCAGTG |
| | | | GACTGGCCCTCATCCACCATAACACCCACCTCTGCTTCG |
| | | | TGCACACGGTGCCCTGGGACCAGCTCTTTCGGAACCCG |
| | | | CA |
| 731 | ERBB2 | 3720435 | AGCAGCGTTCTTGGACTTGTGCAGACTGCCCGTCTCTGT |
| | | | GCACCCTTCTTGACTCAGCACAGCTCTGGCTGGCTTGGC |
| | | | CTCTTGGCATGGCTTCTCTAGCTGGGTCCTACCTGCCTT |
| | | | GGCATCCTTCCCTCCCCCTCTGTTTCTGAAATCTCAGAA |
| | | | CTCTTCCTCTCCCTACATCGGCCCCACCTGTCCCCACCC |
| | | | CTCCAGCCCACAGCCATGCCCACAGCCAGTTCCCTGGTT |
| | | | CACTTGGACCTG |
| 732 | ERBB2 | 3720436 | GCCACCAGCTGTGCGCCCGAGGGCACTGCTGGGGTCCA |
| | | | GGGCCCACCCAGTGTGTCAACTGCAGCCAGTTCCTTCG |
| | | | GGGCCAGGAGTGCGTGGAGGAATGCCGA |
| 733 | ERBB2 | 3720437 | TGGCTGGAGGGGTGCATGGGGCTCCTCTCAGACCCCCT |
| | | | CACCACTGT |
| 734 | ERBB2 | 3720438 | GCTCCCCAGGGAGTATGTGAATGCCAGGCACT |
| 735 | ERBB2 | 3720439 | ACTCGCTGTTACACCTTAGGTAATGCGTTTTCCTCTCTG |
| | | | GGTGCCTCCCATTTTCTGGCTCAAGTCCCTGCCCAGGAT |
| | | | CAAGCTTGGAGGAGGGCCCCGAGGGAGGGGCCACAGA |
| | | | GACTGGGTGAAGAGCAAGGGTGTTTGTCCCAGGAGCAT |
| | | | GGCGAAAATTGCTGCTGGGTGGCCTTGGGAAGCACAAA |
| | | | GGGGACCCAACTAAGGGCCTGATCCTACTGCC |
| 736 | ERBB2 | 3720440 | GTGGCCTGTGCCCACTATAAGGACCCTCCCTTCTGCGTG |
| | | | GCCCGCTGCCCCAGCGGTGTGAAACCTGACCTCTCCTA |
| | | | CATGCCCATCTGGAAGTTTCCAGATGAGGAGGGCGCAT |
| | | | GCCA |
| 737 | ERBB2 | 3720441 | GTGAGTCCAACGGTCTTTTCTGCAGAAAGGAGGACTTT |
| | | | CCTTTCAGGGGTCTTTCTGGGGCTCTTACTATAAAAG |
| 738 | ERBB2 | 3720442 | GGGCTGCCCCGCCGAGCAGAGAGCC |
| 739 | ERBB2 | 3720443 | TTCCACTGTGGAACCTCCTGTCATTTTCCACTTCACCAA |
| | | | GTGACAGAGGACCTGCTCAGATGCTGAGGGGAGGGGA |
| | | | CTGCAAGGAAAGATGGCTAGGAAACCCAGTCCCTCCAC |
| | | | ACCCTAGAGTAACTTGATGCCTTGTGAGGGACACAGGC |
| | | | AAAGTTCAATTC |
| 740 | ERBB2 | 3720444 | GTGGTCTTTGGGATCCTCATCAAGCGACGGCAGCAGAA |
| | | | GATCCGGAAGTACACGATGCGGAGACTGCTGCAGG |
| 741 | ERBB2 | 3720446 | AGCGATGCCCAACCAGGCGCAGATGCGGATCCTGAAAG |
| | | | AGACGGAGCTGAGGAAGGTGAAGGTGCTTGGATCTGGC |
| | | | GCTTT |
| 742 | ERBB2 | 3720447 | GCATCTGGATCCCTGATGGGGAGAATGTGAAAATTCCA |
| | | | GTGGCCATCAAAGTGTTGAGGGAAAACACATCCCCCAA |
| | | | AGCCAACAAAGAAATCTTAG |
| 743 | ERBB2 | 3720449 | TGGGCATCTGCCTGACATCCACGGTGCAGCTGGTGACA |
| | | | CAGCTTATGCCCTATGGCTGCCTCTTAGACCATGTCCGG |
| | | | GAAAACCGCGGACGCCTGGGCTCCCAGGACCTGCTGAA |
| | | | CTGGTGTATGCAGATTGCCA |
| 744 | ERBB2 | 3720452 | ATGTGCGGCTCGTACACAGGGACTTGGCCGCTCGGAAC |
| | | | GTGCTGGTCAAGAGTCCCAACCATGTCAAAATTACAGA |
| | | | CTTCGGGCTGGCTCGGCTGCTGGACATTGACGAGACAG |
| | | | AGTA |
| 745 | ERBB2 | 3720453 | CTGGGTGGAGTGGTGTCTAGCCCATGGGAGAACTCTG |
| 746 | ERBB2 | 3720454 | CTGGAGTCCATTCTCCGCCGGCGGTTCACCCACCAGAG |
| | | | TGA |
| 747 | ERBB2 | 3720455 | ACTGTGTGGGAGCTGATGACTTTTGGGGCCAAACCTTA |
| | | | CGATGGGATCCCAGCCCGGGAGATCCCTGACCTGCTG |
| 748 | ERBB2 | 3720456 | GTGCGTGGCTGAGCTGTGCTGGCTGCCTGGA |
| 749 | ERBB2 | 3720457 | GTTGGATGATTGACTCTGAATGTCGGCCAAGATTCCGG |
| | | | G |
| 750 | ERBB2 | 3720458 | GTTGGTGTCTGAATTCTCCCGCATGGCCAGGGACCCCC |
| | | | AGCGCTTTGTGGT |
| 751 | ERBB2 | 3720460 | TCTACCGCTCACTGCTGGAGGACGATGACATGGGGGAC |
| | | | CTGGTG |
| 752 | ERBB2 | 3720462 | GTGGGGACCTGACACTAGGGCTGGAGCCCTCTGAAGAG |
| | | | GAGGCCCCCAGGTCTCCACTGGCACCCTCCGAAGGGGC |
| | | | TGGCTCCGATGTATTTGATGGTGACCTGGGAATGGGGG |
| | | | CAGCCAAGGGGCTGCAAAGCCTCCCCACACATGACCCC |
| | | | AGCCCTCTACAGCGGTACAGTGAGGACCCCACAGTACC |
| | | | CCTGCCCTCTGAGACTGATGGCTACGTTGCCCCCCTGA |
| 753 | ERBB2 | 3720463 | ATGTGAACCAGCCAGATGTTCGGCCCCAGCCC |
| 754 | ERBB2 | 3720464 | AGCCTTCGACAACCTCTATTACTGGGACCAGGACCCAC |
| | | | CAGAGCGGGGGGCTCCACCCAGCACCTTCAAAGGGACA |
| | | | CCTACGGCAGAGAACCCAGAGTACCTGGGTCTGGACGT |
| | | | GCCAGTGT |
| 755 | ERBB2 | 3720465 | AGAAGGCCAAGTCCGCAGAAGCCCT |
| 756 | ERBB2 | 3720466 | CGACCACTTCCAGGGGAACCTGCCATGCCAGGAACCTG |
| | | | T |
| 757 | ERBB2 | 3720467 | CTTCCTGCTTGAGTTCCCAGATGGCTGGAAGGGGTCCA |
| | | | GCCTCGTTGGAAGAGGAACAGCACTGGGGAGTCTTTGT |
| | | | GGATTCTGAGGCCCTGCCCAATGAGACTCTA |
| 758 | ERBB2 | 3720468 | GGTACTGAAAGCCTTAGGGAAGCTGGCCTGAGAGGGG |
| | | | AAGCGGCCCTAAGGGAGTGTCTAAGAACAAAAGCGAC |
| | | | CCATTCAGAGACTGTCCCTGAAACCTAGTACTGCCC |
| 759 | ERBB2 | 3720469 | AGTATCCAGGCTTTGTACAGAGTGCTTTTCT |
| 760 | GRB7 | 3720477 | CCTCCCTGAAGACGTGGTCCCAGCCGGGTGTC |
| 761 | GRB7 | 3720478 | GGAGAGGGATCCTCTAAATTGTCGAGGCTTCATCTCTCC |
| | | | AGATTGTATGCCCTTCTC |
| 762 | GRB7 | 3720481 | CTGGTTCCGTTAAGCCCCTCTCTTG |
| 763 | GRB7 | 3720482 | ATCTTAGCAGCTCTCCGGAAGACCTTTGCCCAGCCCCTG |
| | | | GGACCCCTCCTGGGACTCCCCGGCCCCCTGATACCCCTC |
| | | | TGCCTG |
| 764 | GRB7 | 3720483 | GTAAAGAGGTCCCAGCCTCTCCTCAT |
| 765 | GRB7 | 3720485 | GAAACTTCGAGAGGAGGAGAGGCGTGCCACCTCCCTCC |
| 766 | GRB7 | 3720486 | TCTCGGGGGCCCCTCCAGTGCAAGGGGGCTGCTCCCCC |
| | | | GCGATGCCAGCCGCCCCCA |
| 767 | GRB7 | 3720487 | TGGGAGATACACAGCCGCTTCCATGGAGGCAGGGGATC |
| | | | TTGGTTAGGAGTCCCTGAGGGTCTAGCAGGTGCGGAAA |
| | | | GGGAATGAATCAC |
| 768 | GRB7 | 3720488 | GCCACAGCTCGCCACGTGTGTGAAATGCTGGTGCAGCG |
| | | | AGCTCACGCCTTGAGCGACGAGACCTG |
| 769 | GRB7 | 3720490 | ACCACGAGTCCGTGGTGGAAGTGCAGGCTGCCTGGCCC |
| | | | GTGGGCGGAGATAGCCGCTTCGTCTTCCGGAAAAACTT |
| | | | CGCCAAGTACGAACTGTTCAAGAGCTCCC |
| 770 | GRB7 | 3720491 | TCTGGGCGCTGGGATGCCCTGATCCTCAACCTGGATGCT |
| | | | GGAGCCCTGATCCCTGACACTTGTCTACCCACAG |
| 771 | GRB7 | 3720492 | TCCAGCTGTCTCGATGCACACACTGGTATATCCCATGAA |
| | | | GACC |
| 772 | GRB7 | 3720494 | TCAGGACGGAAGCTTTGGAAACGCTTTTTCTGCTTCTTG |
| | | | CGCCGATCTGGCCTCTATTACTCCACCAAGGGCACCTCT |
| | | | A |
| 773 | GRB7 | 3720495 | CAGTCCCTGGTCCTTTTAGAAGTTGCCCCTTCTCTGCTG |
| | | | GAACCTCTGAGCCCTTCTCCCCCTGGGCCCCCCAGGCCA |
| | | | GCCACCTCCAGTTTACCATCTCTCCCTACATCCTTGCCT |
| | | | AGCTCACCTGCCCAG |
| 774 | GRB7 | 3720496 | ATCCGAGGCACCTGCAGTACGTGGCAGATGTGAACGAG |
| | | | TCCAACGTGTACGTGGTGACGCAGGGCCGCAAGCTCTA |
| | | | CGGGATGCCCACTGACTTCGGTTTCTGTGTC |
| 775 | GRB7 | 3720498 | CAACAAGCTTCGAAATGGCCACAAGGGGCTTCGGATCT |
| | | | TCTGCAGTGAAGATGAGCAGAGCCGCACCTGCTGGCTG |
| 776 | GRB7 | 3720501 | TACGGGGTGCAGCTGTACAAGAATTACCAGCAGGCACA |
| | | | GTCTCGCCATCTGCATCCATCTTGTTTGGGCTCCCCACC |
| 777 | GRB7 | 3720502 | AGAAGTGCCTCAGATAATACCCTGGTGGCCATGGACTT |
| | | | CTCTGGCCATGCTGGGCGTGTCATTGAGAACCCCCGGG |
| | | | AGGCTCT |
| 778 | GRB7 | 3720505 | CCATCCACCGCACCCAACTCTGGTTCCACGGGCGCATTT |
| | | | CCCGTGAGGAGAGCCAGCGGCTTATTGGACAGCAGGGC |
| | | | T |
| 779 | GRB7 | 3720507 | CTGTTCCTGGTCCGGGAGAGTCAGCGGAACCCCCAGGG |
| | | | CTTTGTCCTCTCTTTGTGCCACCTGCAGAAAGTG |
| 780 | GRB7 | 3720510 | AGGAGGGCCGCCTGTACTTCAGCATGGATGATGGCCAG |
| | | | ACCCGCTTCACTGACCTGCTGCAGCTCGTGGAGTTCCAC |
| | | | CAGCTGAACCGCGGCATCCTGCCGTGCTTGCTGCGCCA |
| | | | TTGCTGCACGCGG |
| 781 | GRB7 | 3720511 | CCCATCCAGTGGACTCTGGGGCGCGGCCACAGGGGACG |
| | | | GGATGAGGAGCGGGAGGGTTCCGCCACTCCAGTTTTCT |
| | | | CCTCTGCTTCTTTGCCTCCCTCAGATAGAAAACAGCCCC |
| | | | CACTCCAGTCCACTCCTGACCCCTCTCCTCAAGGGAAG |
| | | | GCCTTGGGTGGCCCCCTCTCCTTCTCCTAGCTCTGGAGG |
| | | | TGCTGCTCTAGGGCAGGGAATTATGGGAGAAGTGGGGG |
| | | | CAGCCCAGGCGGTTTCACGCCCCACACTTTGTACAGAC |
| | | | CGAGAGGCCAGTTGATCTGCTCTGTTTTA |
| 782 | CDC6 | 3720897 | TGGCCTCACAGCGACTCTAAGACTTGGGGCTCTCTCATT |
| | | | GGCTGTAACTCTTCCACTGGATTGGTAGCAAAAAAAGA |
| | | | GGCGGTGCCCAAGGCGAAAGGCTCTGTGACTACAGCCA |
| | | | ATCAGAATCGAGGCCGGGCTTT |
| 783 | CDC6 | 3720900 | CTGTCTCGGGCATTGAACAAAGCTAAAAACTCCAGTGA |
| | | | TGCCAAACTAGAACCAACAAATGTCCAAACCGTAACCT |
| | | | GTTCTCCTCGTGTAAAAGCCCTGC |
| 784 | CDC6 | 3720901 | GCGATGACAACCTATGCAACACTCCCCATTTACCTCCTT |
| | | | GTTCTCCACCAAAGCAAGGCAAGAAAGAGAATGGTCCC |
| | | | CCTCACTCACATACACTTAAGGGACGAAGATTGGTATT |
| | | | TGACAATCAGCTGACAATTAAGTCTCCTAGCAAAAGAG |
| | | | AACTAGCCAAAGTTCACCAAAACAAAATACTTTCTTCA |
| | | | GTTAGAAAAAGTCAAGAGATCACAACAAATTCTGAGCA |
| | | | GAGATGTCCACTGAAGAAAGAATCTGCATGTGTGAGAC |
| | | | TATTCAAGC |
| 785 | CDC6 | 3720902 | TGCTACCAGCAAGCAAAGCTGGTCCTGAACACAGCTGT |
| | | | CCCAGATCGGCTGCCTGCCAGGGAAAGGGAGATGGATG |
| | | | TCATCAGGAATTTCTTGAGGGAACACATCTGTGGGAAA |
| | | | AAAGCTGGAAGCCTTTACCTTTCTGGTGCTCCTGGAACT |
| | | | GGAAAAACTGCCTGCTTAAGCCGGA |
| 786 | CDC6 | 3720903 | TTTAAAACTATCATGCTGAATTGCATGTCCTTGAGGACT |
| | | | GCCCAGGCTGTATTCCCAGCTATTGCTCAGGAGATTTGT |
| | | | CAGGAAGAGGTATCCAGGCCAGC |
| 787 | CDC6 | 3720904 | GACAGCAAAGGCCAGGATGTATTGTACACGCTATTTGA |
| | | | ATGGCCATGGCTAAGCAATTCTC |
| 788 | CDC6 | 3720905 | GTATTGCTAATACCCTGGATCTCACAGATAGAATTCTAC |
| | | | CTAGGCTTCAAGCTAGAGAAAAATGTAAGCCACAGCTG |
| | | | TTGAACTTCCCACCTTATACCAGAAATCAGATAGTCACT |
| | | | ATTTTGCAAGATCGACTTA |
| 789 | CDC6 | 3720906 | GTATCTAGAGATCAGGTTCTGGACAATGCTGCAGTTCA |
| | | | ATTCTGTGCCCGCAAAGTCTCTGCT |
| 790 | CDC6 | 3720907 | GAGATGTTCGCAAAGCACTGGATGTTTGCA |
| 791 | CDC6 | 3720908 | GTGAGTTACGGCTCTGTTGCATTCTT |
| 792 | CDC6 | 3720910 | AGATGTCAAAAGCCAGACTATTCTCAA |
| 793 | CDC6 | 3720913 | GTAAATCACCTTCTGAGCCTCTGATTCCCAAG |
| 794 | CDC6 | 3720914 | TATCCCAAGTCATCTCAGAAGTTGATGGTAACAGGATG |
| | | | ACCTTGAGCCAAGAAGGAGCACAAGATTCCTTCCCTCT |
| | | | TCAGCAGAAGATCTTGGTTTGCTCTTTGATGCTCTTGAT |
| | | | CAGGCAGTTGAAAATCAAAGAGGTCACTCTG |
| 795 | CDC6 | 3720916 | TACAGTAAAGTCTGTCGCAAACAGCAGGTGGCGGCTGT |
| | | | GGACCAGTCAGAGTGTTTGTCACTTTCAGGGCTCTTGGA |
| | | | AGCCAGGGGCATTTTAGGATTAAAGA |
| 796 | CDC6 | 3720917 | GAAATAGAACATGCTCTGAAAGATA |
| 797 | CDC6 | 3720918 | CCCGAAAGTATTCAGCTGGCATTTAGAGAGCTACAGTC |
| | | | TTCATTTTAGTGCTTTACACATTCGGGCCTGAAAACAAA |
| | | | TATGACCTTTTTTACTTGAAGCCAATGAATTTTAATCTA |
| | | | TAGATTCTTTAATATTAGCACAGAATAATATCTTTGGGT |
| | | | CTTACTATTTTTACCCATAAAAGTGACCAGGTAGACCCT |
| | | | TTTTAATTACATTCACTACTTCTACCACTTGTGTATCTCT |
| | | | AGCCAATGTGCTTGCAAGTGTACAGATCTGTGTAGAGG |
| | | | AATGTGTGTATATTTACCTCTTCGTTTGCTCAAACATGA |
| | | | GTGGGTATTTTTTTGTTTGTTTTTTTTGTTGTTGTTGTTTT |
| | | | TGAGGCGCGTCTCACCCTGTTGCCCAGGCTGGAGTGCA |
| | | | ATGGCGCGTTCTCTGCTCACTACAGCACCCGCTTCCCAG |
| | | | GTTGAAGTGATTCTCTTGCCTCAGCCTCCCGAGTAGCTG |
| | | | GGATTACAGGTGCCCACCACCGCGCCCAGCTAATTTTTT |
| | | | AATTTTTAGTAGAGACAGGGTTTTACCATGTTGGCCAG |
| | | | GCTGGTCTTGAACTCCTGACCCTCAAGTGATCTGCCCAC |
| | | | CTTGGCCTCCCTAAGTGCTGGGATTATAGGCGTGAGCC |
| | | | ACCATGCTCAGCCATTAAGGTATTTTGTTAAGAACTTTA |
| | | | AGTTTAGGGTAAGAAGAATGAAAATGATCCAGAAAAA |
| | | | TGCAAGCAAGTCCACATGGAGATTTGGAGGACACTGGT |
| | | | TAAAGAATTTATTTCTTTGTATAGTATACTATGTTCATG |
| | | | GTGCAGATACTACAACATTGTGGCATTTTAGACTCGTTG |
| | | | AGTTTCTTGGGCACTCCCAAGGGCGTTGGGGTCATAAG |
| | | | GAGACTATAACTCTACAGATTGTGAATATATTTATTTTC |
| | | | AAGTTGCATTCTTTGTCTTTTTAAGCAATCAGATTTCAA |
| | | | GAGAGCTCAAGCTTTCAGAAGTCAATGTGAAAATTCCT |
| | | | TCCTAGGCTGTCCCACAGTCTTTGCTGCCCTTAGATGAA |
| | | | GCCACTTG |
| 798 | MAPT | 3723688 | GCCGGCCTCAGGAACGCGCCCTCTTCGCCGGCGCGCGC |
| | | | CCTCGCAGTCACCGCCACCCACCAGCTCCGGCACCAAC |
| | | | AGCAGCGCCGCTGCCACCGCCCACCTTCTGCCGCCGCC |
| | | | ACCACAGCCACCTTCTCCTCCTCCGCTGTCCTCTCCCGT |
| | | | CCTCGCCTCTGTCGACTATCA |
| 799 | MAPT | 3723690 | TAAGGTTACTGGTGCTTCGGCCACACCCATCTTTCTGAG |
| | | | CCCACTGGACTGGGCGCAGAGGGGGGATTGCCATGGAA |
| | | | ACCACAGGTGTCCGGAGAGGGGATCTTGGGGCTGGCCT |
| | | | CACCCCTTCCCTGCGGAGATTGGGGACCCTGGGGTAGG |
| | | | GGGAGCCGCGCCCAGTCGGCCTCCTGGAGGACACGGGA |
| | | | GGAAGCCCCGAACCCCCGCGCCTGAGGCTGTTTCTGAT |
| | | | TGGCCCCTGGAGGCCGCAGACACGCAGATAGGCGGCCC |
| | | | TGGGTGTATTTTTATTAATATTATGTCCGTACTGATTAA |
| | | | TATTATTTATCTTAAATAAATTTCACCCGTGTCCAAGTT |
| | | | CACCGCGCCCCCAAAACCGAGTCTGGGGCGGCAGGGG |
| | | | GAACTCCTGGCCAACGAATCCATGCCTCGCCCTCCTGTG |
| | | | ATGAACCTGGTACGCACGGTTTTCTGGTTAATTCTATCG |
| | | | CTGAAAACTGGTGCGGGGGGCGCACTTCTGAGACGGAA |
| | | | GAGCATCTAGGAGCTGAATCCTCCACGCGGGTCGCCCA |
| | | | GGTTGATCTGAATTTCTGGGGAATGGCTTGGCTGCCCGC |
| | | | CCGGGACCAGGCCGACCCTCCTTGACGGTGGCGTAGAG |
| | | | GGCTGGAGCCTGGGTACTGCGAGGCTCCTCGCATGGCT |
| | | | GGGCCCGCCGCGAGGGGTTGCAGAGCGGCTCAGGGATC |
| | | | GATTCAAGCATCGTCTCTCCTCCCTCGCCCCCAGACAGA |
| | | | GCTGGGCGCGGGGTTCCCCTTCCAGATGGAGCGAGGGT |
| | | | CTCGGGGTGGCCCCGGAAAAGGGGAGCCCGCGGCCAC |
| | | | GGCTACGTATTGCCATCTCGCGAGCAGAGATGTCACCT |
| | | | CCTGCCTTTGGAGGAAAGGGAGCCCGGTGGGGATGAGC |
| | | | GCATTTAGCCCAATGCTGGGAACAAAGCGCACTCCGCG |
| | | | CTTCTGCGATTTCGCTCCATTTTGAAATGTGTTGGCGCT |
| | | | TTGGTGGGGCCGCTGCGGTGGGCAAGGCCGGGGGCGCT |
| | | | GTTAATGGAGGAACCTCAGGGGGACGGTCCTTCGTAGG |
| | | | AAACTCTATCCTGGCTCTGCGCGCGCTTTAAGGAAATG |
| | | | GC |
| 800 | MAPT | 3723691 | ACCTCGAGGGATGCAGCTTTTGCGCGGATGACGGTGGG |
| | | | GTGCTGAACCAGCCGGTGCGCCTCTGGAAATGTCTGGG |
| | | | CACGGATCCTGGGGCCATCGACGACTCCTCCCCATTCCC |
| | | | AGCAGGCGGGAGCTCTTACATTCCGAG |
| 801 | MAPT | 3723692 | GAAATGTCTTTCCTACCGCGGTTGATTCTGGGGTGTCAT |
| | | | TTTGTGTTTTGTGATGGCTGCTTATATTTACTGTATAAG |
| | | | CATTGTATTTACTGTATAAGCATTGTATTATAATTACTG |
| | | | TATAAGCTGCTTATATTTACTGTATAAGCATCTCCAAAT |
| | | | CCTCCCTCTACGTAAACAAATTAATGGATAAACAGATA |
| | | | AGTGTATCCCCTGCCCCCACCCCTGCTACGCAGGTCCGG |
| | | | AGTGACTCTTGAAGCTCATACATTCCTTGGCCAAGTTTG |
| | | | CTTCTCTAACAGATGTTTATATAGCAATAACCTGGCTTG |
| | | | GCTCTTGGGTTCACCTTTGGACGATTTGGGGAAGGGGC |
| | | | TTGTTGGCTTTGCTGGGTTTTGGATGAGTGACAGTCCAT |
| | | | GACTGTTCCTGCTGGAAGGGCGTGACTTTTAAGTGGTTT |
| | | | CTAATATCAGGCATTGCTCCTCCGACAGGAACAAAAGA |
| | | | AATGGATACTGCCCATAAATTGTTAGAAAACTTAGAAT |
| | | | CGCTTTGATTGAGGAAAGGTTAGATTTATTCCGGTTGGA |
| | | | AAAAGTGGCCTTTCTATTAAACGTGCCCTTTGACCCTCA |
| | | | TGCCCTTGGAGGTCGGTGCCAGCCTGGAGATGGGATAA |
| | | | GATTGTGGTTTTCCTTCTGCCTTTTTAACATCTGTTGTTA |
| | | | CAGTCCATTTGTTGAAAATTTAAAGAAACTGTTTTATTC |
| | | | CACTTTCCCTCAGCATTTATGTGTGTGGTTTCAGTAGCT |
| | | | CTGTGGCTATATGTACGAACACGTGTTATTTTTCCAATT |
| | | | GGACATGTGATAATTTTCCAACTGGACCTTGCCTTCTAT |
| | | | TGATGTA |
| 802 | MAPT | 3723707 | CCCGCCAGGAGTTCGAAGTGATGGAAGATCACGCTGGG |
| | | | ACGTACGGGTTGGGGGACAGGAAAGATCAGGGGGGCT |
| | | | ACACCATGCACCAAGACCAA |
| 803 | MAPT | 3723712 | TGTGACAGCACCCTTAGTGGATGAGGGAGCTCCCGGCA |
| | | | AGCAGGCTGCCGCGCAGCCCCACACGGAGATCCCAGAA |
| | | | GGAACC |
| 804 | MAPT | 3723713 | GCCAGTCCCATGTGACAGTCAAAGCTTCTAACTCCATTC |
| | | | AAAGTTGCAGCCATTCCCCTCGAGGGCTGGCAGGGAGG |
| | | | GGAGGGGTAAGAGAAACAGGAAGGTTCTTACTGAGTTG |
| | | | GTCCTGGTGTGAGCTGCGTCACACTCCCTGCAGAGGTTT |
| | | | CAAGGAGACTCTCTCTCTCTCTGTCTCCATGGGGACCTT |
| | | | ATTTGAATTCTTCTACTCTTACCCCAGCCTGCCATCTCC |
| | | | AGCTATCCTCCCCTGAAGAGCCCTTCTGCTGCGCTGGAT |
| | | | TCTGGTGGCCATGTCATCTCCTCGGCCCCGTGGGAGTCT |
| | | | GAAGATCTGGCTGCAGCCTCACCTCTGAGGTCCTGCTA |
| | | | GTTGCCACCTCTTAAA |
| 805 | MAPT | 3723714 | AGCCTGGAAGACGAAGCTGCTGGTCACGTGACCCAAG |
| 806 | MAPT | 3723715 | CTGGAATTGCCTGCCATGACTTGGGGGTTGGGGGGAGG |
| | | | GACATGGGGTGGGCTCTGCCCTGAAAAGATCATTTGGA |
| | | | CCTGAGCTCTAATTCACAAGTCCAGGAGATTTTAGGGA |
| | | | GTTGGTTCTTATCAAAGGTTGGCTACTCA |
| 807 | MAPT | 3723716 | GCGATTCTCACTGCAGGCTGCCCTGTGGCTGATCCAGG |
| | | | AGCAAGGCCTTAACCATGTCATCCCCAAGCGATTGCTT |
| | | | GTAAACTTTCTTCTGTGCAGCCTTCAACCCTTATTATGA |
| | | | TTTTCTTCTCAGGAACCAAACTGCTGTATTCAAGAAAGG |
| | | | CAGCTTTGTGTAATCATTTATCATAAATATCTTAAGAAA |
| | | | AATCCTAGAGATTCCTAATTTTAGGAAATGGGAGACCT |
| | | | ATGGTACTGATATAATGTGGGCTGGGCTTGTTTTCTGTC |
| | | | ATTTGCTAGATAAATGAACTTGAGAGCCTACTGTAAAA |
| | | | TGTGGAAGCTTCTAGATTGCAGAAGGGCTGGAAAGACA |
| | | | CTGTTCTTTTCTCCCGAGTGATGGGATCTGTCCAGTATT |
| | | | TAGAGCTGCCTCTGAGGCCATCTGATTCTAGGAGACTCT |
| | | | GCCTCGTTGAGGATATTTTGAGGCCTAACTACACATTCC |
| | | | TGCCCCCAGAGAGGTCACAGCCTATAGCAGGCTGATGT |
| | | | TTCTCATGTCAC |
| 808 | MAPT | 3723717 | TAAGCTATGGGAAGGCCTGTATACGAGGGGTGGACTTT |
| | | | TCTTCTGTAAGTGTCCAGAGACCAGGCCTCCTGAAGAG |
| | | | GGCATGGGGGCTTAACTTACCTGGACTACTGTG |
| 809 | MAPT | 3723720 | GCCAATGAGATTAGCGCCCACGTCCAGCCTGGACCCTG |
| | | | CGGAGAGGCCTCTGGGGTCTCTGGGCCGTGCCTCGGGG |
| | | | AGAAAGAGCCAGAAGCTCCCGTCCCGCTGACCGCGAGC |
| | | | CTTCCTCAGCACCGTCCCGTTTGCCCAGCGCCTCCTCCA |
| | | | ACAGGAGGCCCTCAGGAGCCCTCCCTGGAGTGGGGACA |
| | | | AAAAGGCGGGGACTGGGCCGAGAAGGGTCCGGCCTTTC |
| | | | CGAAGCCCGCCACCACTGCGTATCTC |
| 810 | MAPT | 3723721 | CTCATGTCCGGCATGCCTGGGGCTCCCCTCCTGCCTGAG |
| | | | GGCCCCAGAGAGGCCACACGCCAACCTTCGGGGACAG |
| | | | GACCTGAGGACACAGAGGGCGGCCGCCACGCCCCTGA |
| | | | GCTGCTCAAGCACCAGCTTCTAGGAGACCTGCA |
| 811 | MAPT | 3723722 | ATGAAGACCGCGACGTCGATGAGTCCTCCCCCCAAGAC |
| | | | TCCCCTCCCTCCAAGGCCTCCCCAGCCCAAGATGGGCG |
| | | | GCCTCCCCAGACAGCCGCCAGAGAAGCCACC |
| 812 | MAPT | 3723725 | ACGGGACTGGAAGCGATGACAAAAAAGCCAA |
| 813 | MAPT | 3723731 | ACATCCACACGTTCCTCTGCTAAAACCTTGAA |
| 814 | MAPT | 3723732 | CTCAGACCCTCTGATCCAACCCTCCAG |
| 815 | MAPT | 3723733 | TCTTCTGTCACTTCCCGAACTGGCAG |
| 816 | MAPT | 3723735 | GGGCTGATGGTAAAACGAAGATCGCCACACCGCGG |
| 817 | MAPT | 3723736 | CCAGGCCAACGCCACCAGGATTCCAGCAAAAACCCCGC |
| | | | CCGCTCCAAAGACACCACCCA |
| 818 | MAPT | 3723737 | CCAAGTCGCCGTCTTCCGCCAAGAGCCGCCTGCAGACA |
| | | | GCCCCCGTGCCCATGCCAGACCTGAAGAATGTCAAGTC |
| | | | CAAGATCGGCTCCACTGAGAACCTGAAGCACCAGCCGG |
| | | | GAGGCG |
| 819 | MAPT | 3723740 | AGAAGCTGGATCTTAGCAACGTCCAGTCCAAGTGTGGC |
| | | | TCAAAGGATAATATCAAACACGTCCCGGGAGGC |
| 820 | MAPT | 3723743 | TAGTCTACAAACCAGTTGACCTGAGCAAGGTGACCTCC |
| | | | AAGTGTGGCTCATTAGGCAACATCCATCATA |
| 821 | MAPT | 3723746 | CAGAGTCCAGTCGAAGATTGGGTCCCTGGACAATATCA |
| | | | CCCACGTCCCTGGCGGAGGAAAT |
| 822 | MAPT | 3723747 | TCGTGTACAAGTCGCCAGTGGTGTCTGGGGACACGTCT |
| | | | CCACGGCATCTCAGCAATGTCTCCTCCACCGGCAGCAT |
| | | | CGACATGGTAGACTCGCCCCAGCTCGCCACGCTAGCTG |
| | | | ACGAGGTGTCTGCCTCCC |
| 823 | MAPT | 3723748 | CCCTGGGGCGGTCAATAATTGTGGAGAGGAGAGAATGA |
| | | | GAGAGTGTGGAAAAAAAAAGAATAATGACCCGGCCCC |
| | | | CGCCCTCTGCCCCCAGCTGCTCCTCGCAGTTCGGTTAAT |
| | | | TGGTTAATCACTTAACCTGCTTTTGTCACTCGGCTTTGG |
| | | | CTCGGGACTTCAAAATCAGTGATGGGAGTAAGAGCAAA |
| | | | TTTCATCTTTCCAAATTGATGGGTGGGCTAGTA |
| 824 | MAPT | 3723749 | ACATGGCCACATCCAACATTTCCTCAGG |
| 825 | MAPT | 3723750 | TGCTTCTGGGGGATTTCAAGGGACTGGGGGTGCCAACC |
| | | | ACCTCTGGCCCTGTTGTGGGGGTGTCACAGAGGCAGTG |
| | | | GCAGCAACAAAGGATTTGAAACTTGGTGTGTTCGTGGA |
| | | | GCCACAGGCAGACGATGTCAACCTTGT |
| 826 | MAPT | 3723751 | CCTCCTTGCCGCTGGGAGAGCCAAGGCCTATGCCACCT |
| | | | GCAGCGTCTGAGCGGCCGCCTGTCCTTGGTGGCCGGGG |
| | | | GTGGGGGCCTGCTGTGGGTCAGTGTGCCACCCTCTGCA |
| | | | GGGCAGCCTGTGGGAGAAGGGACAGCGGGTAAAAAGA |
| | | | GAAGGCAAGCTGGCAGGAGGGTGGCACTTCGTGGATG |
| | | | ACCTCCTTAGAAAAGACTGACCTTGATGTCTTGAGAGC |
| | | | GCTGG |
| 827 | MAPT | 3723752 | TTGCAGACCTGGGACTTTAGGGCTAACCAGTTCTCTTTG |
| | | | TAAGGACTTGTGCCTCTTGGGAGACGTCCACCCGTTTCC |
| | | | AAGCCTGGGCCACTGGCATCTCTGGAGTGTGTGGGGGT |
| | | | CTGGGAGGCAGGTCCCGAGCCCCCTGTCCTTCCCACGG |
| | | | CCACTGCAGTCACCCCGTCTGCGCCGCTGTGCTGTTGTC |
| | | | TGCCGTGAGAGCCCAATCACTGCCTATACCCCTCATCAC |
| | | | ACGTCACAATGTCCCGAATTCCCAGCCTCACCACCCCTT |
| | | | CTCAGTAATGACCCTGGTTGGTTGCAGGAGGTACCTAC |
| | | | TCCATACTGAGGGTGAAATTAAGGGAAGGCAAAGTCCA |
| | | | GGCACAAGAGTGGGACCCCAGCCTCTCACTCTCAGTTC |
| | | | CACTCATCCAACTGGGACCCTCACCACGAATCTCATGA |
| | | | TCTGATTCGGTTCCCTGTC |
| 828 | MAPT | 3723753 | CCACTTGCACCCTAGCTTGTAGCTGCCAACCTCCCAGAC |
| | | | AGCCCAGCCCGCTGCTCAGCTCCACATGCATAGTATCA |
| | | | GCCCTCCACACCCGACAAAGGGGAACACACCCCCTTGG |
| | | | AAATGGTTCTTTTCCCCCAGTCCCAGCTGGAAGCCATGC |
| | | | TGTCTGTTCTGCTGGAGCAGCTGAACATATACATAGAT |
| | | | GTTGCCCTGCCCTCCCCATCTGCACCCTGTTGAGTTGTA |
| | | | GTTGGATTTGTCTGTTTATGCTTGGATTCACCA |
| 829 | MAPT | 3723754 | TTCTAGCAGCTAAGGAGGCCGTTCAGCTGTGACGAAGG |
| | | | CCTGAAGCACAGGATTAGGACTGAAGCGATGATGTCCC |
| | | | CTTCCCTACTTCCCCTTGGGGCTCCCTGTGTCAGGGCAC |
| | | | AGACTAGGTCTTGTGGCTGGTCTGGCTTGCGGCGCGAG |
| | | | GATGGTTCTCTCTGGTCATAGCCCGAAGTCTCATGGCAG |
| | | | TCCCAAAGGAGGCTTACAACTCCTGCATCACAAGAAAA |
| | | | AGGAAGCCACTGCCAGCTGGGGGGATCTGCAGCTCCCA |
| | | | GAAGCTCCGTGAGCCTCAGCCACCCCTCAGACTGGGTT |
| | | | CCTCTCCAAGCTCGCCCTCTGGAGGGGCAGCGCAGCCT |
| | | | CCCACCAAGGGCCCTGCGACCACAGCAGGGATTGGGAT |
| | | | GAATTGCCTGTCCTGGATCTGCTCTAGAGGCCCAAGCT |
| | | | GCCTGCCTGAGGAAGGATGACTTGACAAGTCAGGAGAC |
| | | | ACTGTTCCCAAAGCCTTGACCAGAGCACCTCAGCCCGC |
| | | | TGACCTTGCACAAACTCCATCTGCTGCCATGAGAAAAG |
| | | | GGAAGCCGCCTTTGCAAAACATTGC |
| 830 | MAPT | 3723756 | GTGTCTGCTGCTCCCTAGTCTGGGCC |
| 831 | MAPT | 3723757 | CCGGCCCTCATTGAATGCGGGGTTAATTTAACTCAGCCT |
| | | | CTGTGTGAGTGGATGATTCAGGTTGCCAGAGACAGAAC |
| | | | CCTCAGCTTAGCATGGGAAGTAGCTTCCCTGTTGACCCT |
| | | | GAGTTCATCTGAGGTTGGCTTGGAAGGTGTGGGCACCA |
| | | | TTTGGCCCAGTTCTTACAGCTCTGAAGAGAGCAGCAGG |
| | | | AATGGGGCTGAGCAGGGAAGACAACTTTCCATTGAAGG |
| | | | CCCCTTTCAGGGCCAGAACTGTCCCTCCCACCCTGCAGC |
| | | | TGCCCTGCCTCTGCCCATGAGGGGTGAGAGTCAGGCGA |
| | | | CCTCATGCCAAGTGTA |
| 832 | BIRC5 | 3736291 | GGGTGGACCGCCTAAGAGGGCGTGCGCTCCCGAC |
| 833 | BIRC5 | 3736292 | GCGGCGCGCCATTAACCGCCAGATTTGAATCGCGGGAC |
| | | | CCGTTGGCAG |
| 834 | BIRC5 | 3736293 | TGCCCCGACGTTGCCCCCTGCCTGGCA |
| 835 | BIRC5 | 3736294 | TTTCTCAAGGACCACCGCATCTCTACATTCAAGAACTGG |
| | | | CCCTTCTTG |
| 836 | BIRC5 | 3736295 | TGGCTTCATCCACTGCCCCACTGAGAACGAGCCAGACT |
| | | | TGGCCCAGTGTTTCTTCTGCTTCAAGGAGCTGGAAGGCT |
| | | | GGGAGCCAGATGACGACCCC |
| 837 | BIRC5 | 3736296 | CCTCGATGGGCTTTGTTTTGAACTGAGTTGTCAAAAGAT |
| | | | TTGAGTTGCAAAGACACTTAGTATGGGAGGGTTGCTTT |
| | | | CCACCCTCATTGCTTCTTAAACAGCTGTTGTGAACGGAT |
| | | | ACCTCTCTATATGCTGGTGCCTTGGTGATG |
| 838 | BIRC5 | 3736298 | GCTTGGGCAAAGCACTGATGCCATCAACTTCAGACTTG |
| | | | ACGTCTTACTCCTGAGGCAGAGCAGGGTGTGCCTGTGG |
| | | | AGGGCGTGGGGAGGTGGCCCGTGGGGAGTGGACTGCC |
| | | | GCTTTAATCCCTTCAGCTGCCTTTCCGCTGTTGTTTTGAT |
| | | | T |
| 839 | BIRC5 | 3736299 | ACATAAAAAGCATTCGTCCGGTTGCGCTTTCCTTTCTGT |
| | | | CAAGAAGCAGTTTGAAGAATTAACCCTTGGTGAATTTT |
| | | | TGAAACTGGACAGA |
| 840 | BIRC5 | 3736301 | AAGAATTTCTGTTCGAGGAAGAGCCTGATGTTTGCCAG |
| | | | GGTCTGTTTAACTGGACATGAA |
| 841 | BIRC5 | 3736303 | AGAATTTGAGGAAACTGCGGAGAAAGTGCGCCGTGCCA |
| | | | TCGAGCAGCTG |
| 842 | BIRC5 | 3736304 | GGCTGCACCACTTCCAGGGTTTATTCCCTGGTGCCACCA |
| | | | GCCTTCCTGTGGGCCCCTTAGCAATGTCTTAGGAAAGG |
| | | | AGATCAACATTTTCAAATTAGATGTTTCAACTGTGCTCT |
| | | | TGTTTTGTCTTGAAAGTGGCACCAGAGGTGCTTCTGCCT |
| | | | GTGCAGCGGGTGCTGCTGGTAACAGTGGCTGCTTCTCTC |
| | | | TCTCTCTCTCTTTTTTGGGGGCTCATTTTTGCTGTTTTGA |
| | | | TTCCCGGGCTTACCAGGTGAGAAGTGAGGGAGGAAGA |
| | | | AGGCAGTGTCCCTTTTGCTAGAGCTGACAGCTTTGTTCG |
| | | | CGTGGGCAGAGCCTTCCACAGTGAATGTGTCTGGACCT |
| | | | CATGTTGTTGA |
| 843 | BIRC5 | 3736305 | GGTTCCTTATCTGTCACACCTGTGCCTCCTCAGAGGACA |
| | | | GTTTTTTTGTTGTTGTGTTTTTTTGTTTTTTTTTTTTTGGT |
| | | | AGATGCATGACTTGTGTGTGATGAGAGAATGGAGACAG |
| | | | AGTCCCTGGCTCCTCTACTGTTTAACAACATGGCTTTCT |
| | | | TATTTTGTTTGAATTGTTAATTCACAGAATAGCACAAAC |
| | | | TACAATTA |
| 844 | BIRC5 | 3736306 | AACGGGGTGAACTTCAGGTGGATGAGGAGACAGAATA |
| | | | GAGTGATAGGAAGCGTCTGGCAGATACTCCTTTTGCCA |
| | | | CTGCTGTGTGATTAGACAGGCCCAGTGAGCCGCGGGGC |
| | | | ACATGCTGGCCGCTCCTCCCTCAGAAAAAGGCAGTGGC |
| | | | CTAAATCCTTTTTAAATGACTTGGCTCGATGCTGTGG |
| 845 | BIRC5 | 3736307 | CGTGTGTCTGTCAGCCCAACCTTCACATCTGTCACGTTC |
| | | | TCCACACGGGGGAGAGACGCAGTCCGCCCAGGTCCCCG |
| | | | CTTTCTTTGGAGGCAGCAGCTCCCGCAGGGCTGAAGTC |
| | | | TGGCGTAAGATGATGGATTTGATTCGCCCTCCTCCCTGT |
| | | | CATAGAGCTGCAGGGTGGATTGTTACAGCTTCGCTGGA |
| | | | AACCTCTGGAGGTCATC |
| 846 | BIRC5 | 3736308 | TGTTCCTGAGAAATAAAAAGCCTGTCATTTC |
| 847 | BIRC5 | 3736309 | GTTTTTCATCGTCGTCCCTAGCCTGCCAACAGCCATCTG |
| | | | CCCAGACAGCCGCAGTGAGGATGAGCGTCCTGGCAGAG |
| | | | ACGCAGTTGTCTCTGGGCGCTTGCCAGAGCCACGAACC |
| | | | CCAGACCTGTTTGTATCATCCGGGCTCCTTCCGGGCAGA |
| | | | AACAACTGAAAATGCACTTCAGACCCACTTATTTCTGCC |
| | | | ACATCTGAGTCGGCCTGAGATAGACTTTTCCCTCTAAAC |
| | | | TGGGAGAATATCACAGTGGTTTTTGTTAGCAGAAAATG |
| | | | CACTCCAGCCTCTGTACTCATCTAAGCTGCTTA |
| 848 | BIRC5 | 3736310 | CTGTGCTGTGGGCAGGGCTGAGCTGGAGCCGCCCCTCT |
| | | | CAGCCCGC |
| 849 | BIRC5 | 3736311 | ATCCTTAAAACCAGACCCTCATGGCTACCAGCACCTGA |
| | | | AAGCTTCCTCGACATCTGTTAATAAAGCCGTAGGCCCTT |
| | | | GTCTAAGTGC |
| 850 | BIRC5 | 3736312 | CCGCCTAGACTTTCTTTCAGATACATGTCCACATGTCCA |
| | | | TTTTTCAGGTTCTCTAAGTTGGAGTGGAGTCTGGGAAGG |
| | | | GTTGTGAATGAGGCTTCTGGGCTATGGGTGAGGTTCCA |
| | | | ATGGCAGGTTAGAGCCCCTCGGGCCAACTGCCATCCTG |
| | | | GAAAGTAGAGACAGCAGTGCCCGCTGCCCAGAAGAGA |
| | | | CCAGCAAGCCAAACTGGAGCCCCCATTGCAGGCTGTCG |
| | | | CCATGTGGAAAGAGTA |
| 851 | KRT14 | 3757155 | TAGGAGGCCCCCCGTGTGGACACAG |
| 852 | KRT14 | 3757156 | TCCAGCCGCCAAATCCGCACCAAGGTCATGGA |
| 853 | KRT14 | 3757157 | TGAGCTCTAGTGCTGTCACCCAGTTTCCCTTGTGAACCT |
| | | | CCTTGGGTGGAAGAAGCTATTTTCTAAACCCTCCTTAGG |
| | | | GCTAGGAGAGGCAGCCCCCACCTCTTGCTTCTACGTGG |
| | | | TGTCTGTGGCAGATCCTATTGCTGTTGTGGTCAGCACCA |
| | | | TGAACAGGGCCCTACAGGGCTCTTCCCACTGAGACCAC |
| | | | TCCATTGGGTGAATATGGATGGAACCAGCCAGGTGTGA |
| | | | GCTCTTAGGAAGCTCTAATCTGAGGGCAAAGACTCTGT |
| | | | CTCTGACCTTTGGGAGCCCTCGTCTGAAAGAAATG |
| 854 | KRT14 | 3757160 | CTGGAGGAGACCAAAGGTCGCTACTGCATGCAGCTGGC |
| | | | CCAGATCCAGGAGATGATTGGCAGCGT |
| 855 | KRT14 | 3757162 | CAAGAGCGAGATCTCGGAGCTCCGGCGCACCATGCAGA |
| | | | AC |
| 856 | KRT14 | 3757164 | GGAGATGGACGCTGCACCTGGCGTGGACCTGAGCCGCA |
| | | | TTCTGAACGAGATGCGTGACCAGTAT |
| 857 | KRT14 | 3757167 | GATGGGTGTCTCATACCTTTTCTCTGGGGTCATTCCAG |
| 858 | KRT14 | 3757169 | AGCCACAGTGGACAATGCCAATGTCCTTCTGCAGATTG |
| | | | ACAATGCCCGTCTGGCCGCGGATGACTTCCGCACCAA |
| 859 | KRT14 | 3757172 | TGGCCTTGGTGCTGGCTTGGGTGGTGGCTTTGGTGGTGG |
| | | | CTTTGCTGGTG |
| 860 | KRT14 | 3757174 | ACCCGAGCACCTTCTCTTCACTCAGCCAACTGCTCGCTC |
| | | | GCTCA |
| 861 | KRT14 | 3757199 | GAGAAGGTGACCATGCAGAACCTCAATGACCGCCTGGC |
| | | | C |
| 862 | KRT17 | 3757215 | GGACTCAGCTACCCCGGCCGGCCAC |
| 863 | KRT17 | 3757218 | CCAGGAATACAAAATCCTGCTGGAT |
| 864 | KRT17 | 3757219 | AAGAGTGAGATCTCGGAGCTCCGGCGCACC |
| 865 | KRT17 | 3757221 | AGGTGGGTGGTGAGATCAATGTGGAGATGGACGCTGCC |
| | | | CCAGGCGTGGACCTGAGCCGCATCCTCAACGAGATGCG |
| | | | TGAC |
| 866 | KRT17 | 3757222 | GTGCACCGGGATTAGTCACCTTAGAGGGCTTCCCTGTCT |
| | | | GCAGAGCCCTGATCCTTGGGGTCCAGTGTGCAGGGCAG |
| | | | ACTCCTCTTTGTACCACACTGCTTCTCTGTACACAAGGA |
| | | | ACCTC |
| 867 | KRT17 | 3757223 | CTGGCCAGAGCCGACCTGGAGATGCAGATTGAGAACCT |
| | | | CAAGGAG |
| 868 | KRT17 | 3757224 | CCCTGCGCCTGAGTGTGGAGGCCGACATCAATGGCCTG |
| 869 | KRT17 | 3757227 | GTCTGGCTGCTGATGACTTCCGCAC |
| 870 | KRT17 | 3757228 | GGTACTGAGTATCGGGGGAAGAAGA |
| 871 | KRT17 | 3757230 | GGCAGCAGCTTTGGGGGTGTTGATGGGCTGCTGGCTGG |
| | | | AGGTGAGAAGGCCACCATGCAG |
| 872 | KRT17 | 3757231 | GTCCCGCACCTCCTGCCGGCTGTCTGGCGGCCTGGGTGC |
| | | | CGGCTCCTGCAGGCTGGGATCTGCTGGCGGCCTGGGCA |
| | | | GCACCCTCGGGGGTAGCAGCTACTCCAGCTGCTACAGC |
| | | | TTTGGCTCTGGTGGTG |
| 873 | KRT17 | 3757234 | ACACGCACGGCACTCAGCACGAGGATTTGGAGA |
| 874 | TYMS | 3775844 | TTGGCCTGCCTCCGTCCCGCCGCGCCAC |
| 875 | TYMS | 3775845 | CTGCCTCCGTCCCGCCGCGCCACTT |
| 876 | TYMS | 3775846 | GCCTGTGGCCGGCTCGGAGCTGCCGC |
| 877 | TYMS | 3775850 | CGCTTCGCAGCGTTTTCAAAAACTGGAGCGAAAGTGAT |
| | | | GTGGGCGGGGCAAAGGCGGCGGGAAGAGGAGAGCACT |
| | | | GAAGCTGGCGCGGGAACTTGGTTTCCTGGTGGCCTCCC |
| | | | ATCCAATCCCCACGAACCAGCTTTCCTCTTAAACCTTGA |
| | | | AAAGAGAAATTCGGGAGTTCGAGTATAAGTTCTTAGTC |
| | | | GTCCTTTCCTCTTTCCTTTCCGACAGGAGCACCCCAGGC |
| | | | AAAAAATGTCTCGCGGGTCATTGGCGCCAGGCTTTCAG |
| | | | GGGACAGTGGGGCGGGGCGGGGTGGGCACAGGACGTT |
| | | | AGGCAGCCGTTGGC |
| 878 | TYMS | 3775851 | CGTCCTGCCGTCCTGGATCCTGCGCCAGCTGCG |
| 879 | TYMS | 3775853 | CTCTGCTGACAACCAAACGTGTGTTCTGGAAGGGTGTT |
| 880 | TYMS | 3775855 | GCTGTCTTCCAAGGGAGTGAAAATCTGGGATGCCAATG |
| | | | GATCCCGAGACTTTTTGGACAGCCTGGGATTCTCCACCA |
| | | | GA |
| 881 | TYMS | 3775856 | AGGACAGGGAGTTGACCAACTGCAAAGAGTGATTGAC |
| | | | ACCATCAAAACCAACCCTGACGACAGAAGAATCATC |
| 882 | TYMS | 3775857 | AAGCAATCTGGTTTTGTGCAGAGGCACCTGAGGGAGGC |
| | | | AGGACCCTGGGAACTTCCCCCAGCCACATGGTTGATTG |
| | | | TGTGACGTTGG |
| 883 | TYMS | 3775861 | CTCTGCCAGTTCTATGTGGTGAACAGTGAGCTGTCCTGC |
| | | | CAGCTGTACCAGAGATCGGGAGACATGGGCCTCGGTGT |
| | | | GCCTTTCAACATCGCCAGCTACGCCCTGCTCACGTACAT |
| | | | GATTGCGCACATCACGGGCCTGAA |
| 884 | TYMS | 3775862 | GGAGATGCACATATTTACCTGAATCACATCGAGCCACT |
| | | | GAAAATT |
| 885 | TYMS | 3775864 | CCAAAGCTCAGGATTCTTCGAAAAGTTGAGAAAATTGA |
| | | | TGACTTCAAAGCTGAAGACTTTCAGATTGAAGGGTACA |
| | | | ATCCGCATCCAACTATTAAAATGGA |
| 886 | TYMS | 3775865 | AGGAGCTCGAAGGATATTGTCAGTCTTTAGGGGTTGGG |
| | | | CTGGATGCCGAGGTAAAAGTTCTTTTTGCTCTAAAAGA |
| | | | AAAAGGAACTAGGTCAAAAATCTGTCCGTGACCTATCA |
| | | | GTTATTA |
| 887 | TYMS | 3775866 | CACTGAGGGTATCTGACAATGCTGAGGTTATGAACAAA |
| | | | GTGAGGAGAATGAAATGTATGTGCTCTTAGCAAAAACA |
| | | | TGTATGTGCATTTCAATCCCACGTACTTATAAAGAAGGT |
| | | | TGGTGAATTTCACAAGCTATTTTTGGAATATTTTTAGAA |
| | | | TATTTTAAGAATTTCACAAGCTATTCCCTCAAATCTGAG |
| | | | GGAGCTGAGTAACACCATCGATCATGATGTAGAGTGTG |
| | | | GTTA |
| 888 | TYMS | 3775867 | TTGTTCATTCTGTACTGCCACTTATCTGCTCAGTTCCTTC |
| 889 | NDC80 | 3776145 | AGCGCCGGCGGAGAATTTCAAATTCGAACGGCTTTGGC |
| | | | GGGCCGAGGAAGGACCTGGTGTTTTGATGACCGCTGTC |
| | | | CTGTCTAGCAGATACTTGCACGGTTTACAGAAATTCGGT |
| | | | CC |
| 890 | NDC80 | 3776147 | ATGAAGCGCAGTTCAGTTTCCAGCGGTGGTGCTGGCCG |
| | | | CCTCTCCATGCAGGAGTTAAGATCCCAGGATGTAAATA |
| | | | AACAAGGCCTCTATACCCCTCA |
| 891 | NDC80 | 3776149 | GTGGACATGGATCCCGGAATAGTCAACTTGGTATATTTT |
| | | | CCAGTTCTGAGAAAATCAAGGACCCGAGACCACTTAAT |
| | | | GACAAAGCATTCATTC |
| 892 | NDC80 | 3776150 | CTTACAGAAAATGGTTATGCACATAATGTGTCCATGAA |
| | | | ATCTCTACAAGCTCCCTCTGTTAAAGACTTCCTGAAGAT |
| | | | CTTCACATTTCTTTATGGCTTCCTGTGCCCCTCATACGA |
| | | | ACTTCCTGACACAAAGT |
| 893 | NDC80 | 3776151 | CTCCTCATACATGGCCTCACATTGTGGCAGCCTTAGTTT |
| | | | GGCTAATAGACTGCATCAAG |
| 894 | NDC80 | 3776152 | TGAAATGTATACATGGGAAAGGGTTTTTTTCCTCAAAA |
| | | | AAAATATTTTCTCTCCCAGTCTTTTGACAGTATTCTCAA |
| | | | AGTCTGCTTCAGAGTTTTCATTTTTCAAAGCACATTTGA |
| | | | TTTTAAG |
| 895 | NDC80 | 3776154 | ATGAAAGAAAGCTCACCTTTATTTGATGATGGG |
| 896 | NDC80 | 3776155 | CTACACCATAAAATGCTATGAGAGTTTTATGAGTGGTG |
| | | | CCGACAGCTTTGATGAGAT |
| 897 | NDC80 | 3776156 | GCTTTTAAGCTGGAATCATTAGAAGCAAAAAACAGAGC |
| | | | ATTGAATGAACAGATTGCAAGATTGGAACAAGA |
| 898 | NDC80 | 3776157 | AATCGTCTAGAGTCGTTGAGAAAACTGAAGGCTTCCTT |
| | | | ACAAGGAGATGTTCAAAAGTATCAGGCATACATGAGCA |
| | | | ATTTGGAGTCTCATTCAGCCATTCTTGACCAGAAATTAA |
| | | | ATGGTCTCAATGAGGAAATTGC |
| 899 | NDC80 | 3776159 | AACCAGAAGTACTCAGTTGCAGACATTGAGCGAATAAA |
| | | | TCATGAAAGAAATGAATTGCAGCAGACTATTAATAAAT |
| | | | TAACCAAGGACCTGGAAGCTGAACAACAGAAGTTGTGG |
| | | | AATGAGGAGTTA |
| 900 | NDC80 | 3776161 | CAGAGTATCACAAATTGGCTAGAAAATTAAAACTTATT |
| | | | CCTAAAGGTGCTGAGAATTCCAAAGGTTATGACTTTGA |
| | | | AATTAAGTTTAATCCCGAGGCTGGTGCCAACTGCCTTGT |
| | | | CAAATACA |
| 901 | NDC80 | 3776162 | GTACCTCTTAAGGAACTCCTGAATGAAACTGAAGAAGA |
| | | | AATTAATAAAGCCCTAAATAAAAAAATGGGTTTGGAGG |
| | | | ATACTTTAGAACA |
| 902 | NDC80 | 3776163 | TTGAATGCAATGATAACAGAAAGCAAGAGAAGTGTGA |
| | | | GAACTCTGAAAGAAGAAGTTCAAAAGCTGGATGATCTT |
| | | | TACCAACAAAAA |
| 903 | NDC80 | 3776165 | ACCTGCTAGAAAGTACTGTTAACCAGGGGCTCAGTGAA |
| | | | GCTATGAATGAATTAGATGCTGTTC |
| 904 | NDC80 | 3776166 | TAGTTGTGCAAACCACGACTGAAGAAAGACGAAAAGT |
| | | | GGGAAATAACTTGCAACGTCTGTTAGAGATGGTTGCTA |
| | | | CACATGTTG |
| 905 | NDC80 | 3776167 | CTGGGGTGAAGCAGCCGCATGCTAAGGAACACCAAGG |
| | | | ACTGCCAGGAGCCGCCAGCAACTGGGGAGAGACGAAG |
| | | | AAGGATTCTTCCCTAGAGCCTTCAGAGAGACCATGGCC |
| | | | CTGCTGACGTCTTGATTTCAAACTTCCGGCCTCCAGAGC |
| | | | TGAAAGAGTACATTTCTGTTGTTTTAAGCCACCTAGTTT |
| | | | GTGGCAATTTGTTACAGTATCAGTATTTGAAATCGCAA |
| | | | AAAAATCAACAAAAACAACAAGAAAAAATAATGTGGC |
| | | | ATGTTAGTTTCCCA |
| 906 | NDC80 | 3776168 | AACATCTTGAGGAGCAGATTGCTAAAGTTGATAGAGAA |
| | | | TATGAAGAATGCATGTCAGAAGATCTCTCGGAAAATAT |
| | | | TAAAGAGATTAGAGATAAGTATGAGAAGAAAGCTACTC |
| | | | TAATTAAGTCTTCTGAAG |
| 907 | NDC80 | 3776169 | TATATCCATAGTGAATAAAATTGTCTCAGTAAA |
| 908 | SLC39A6 | 3804200 | CAAAATGTTCGTGCGGGTATATACCAGATGAGTACAGT |
| | | | GAGTAGTTTTATGTATCACCAGACTGGGTTATTGCCAAG |
| | | | TTATATATCACCAAAAGCTGTATGACTGGATGTTCTGGT |
| | | | TACCTGGTTTACAAAATTATCAGAGTAGTAAAACTTTG |
| | | | ATATATATGAGGATATTAAAACTACACTAAGTATCATTT |
| | | | GATTCGATTCAGAAAGTACTTTGATATCTCTCAGTGCTT |
| | | | CAGTGCTATCATTGTGAGCAATTGTCTTTTATATACGGT |
| | | | ACTGTAGCCATACTAGGCCTGTCTGTGGCATT |
| 909 | SLC39A6 | 3804201 | AGAACTGCTGGTGTTTAGGAATAAGAAT |
| 910 | SLC39A6 | 3804202 | AGTTTCAGTAGGTCATAGGGAGATGAGTTTGTATGCTG |
| | | | TACTATGCAGCGTTTAAAGTTAGTGGGTTTTGTGATTTT |
| | | | TGTATTGAATATTGCTGTCTGTTACAAAGTCAGTTAAAG |
| | | | GTACGTTTTAATATTTAAGTTATTCTATCTTGGAGATAA |
| | | | AATCTGTATGTGCAATTCACCGGTATTA |
| 911 | SLC39A6 | 3804203 | AGAATGCTGGGATGCTTTTGGGTTTTGGAATTATGTTAC |
| | | | TTATTTCCATATTTGAACATAAAATCGTGTTTCGTATAA |
| | | | ATTTCTAG |
| 912 | SLC39A6 | 3804204 | ATGCTGCACAATGATGCTAGTGACCATGGATGTAGCCG |
| | | | CTGG |
| 913 | SLC39A6 | 3804206 | GCATGACCGTTAAGCAGGCTGTCCTTTATAATGCATTGT |
| | | | CAGCCATGCTGGCGTATCTTGGAATGGCAACAGGAATT |
| | | | TTCATTGGTCATTATGCTGAAAATGTTTCTATGTGGATA |
| | | | TTTGCACTTACTGCTGGCTTATTCATGTATGTTGCTCTG |
| | | | GTTGATATG |
| 914 | SLC39A6 | 3804207 | GTTGCTGTGTTCTGTCATGAGTTGCCTCATGAATT |
| 915 | SLC39A6 | 3804208 | GAGCTGAAAGATGCCGGCGTCGCCACTCTGGCCTGGAT |
| | | | GGTGATAATGGGTGATGGCCTGCACAATTTCAGCGATG |
| | | | GCCTAGCAATTG |
| 916 | SLC39A6 | 3804209 | ACCATCATATTCTCCATCATCACCACCACCAAAACCACC |
| | | | ATCCTCACAGTCACAGCCAGCGCTACTCTCGGGAG |
| 917 | SLC39A6 | 3804210 | CCACAGGAAGTCTACAATGAATATGTACCCAGAGGGTG |
| | | | CAAGAATAAATGCCATTCACATTTCCACGATACACTCG |
| | | | GCCAGTCAGACGATCTCATTCACCA |
| 918 | SLC39A6 | 3804212 | TGATGTGGAGATTAAGAAGCAGTTGTCCAAGTATGAAT |
| | | | CTCAACTTTCAACAAATGAGGAGA |
| 919 | SLC39A6 | 3804213 | TGCCTATTTTGATTCCACGTGGAAGGGTCTAACAGCTCT |
| | | | AGGAGGCCTGTATTTCATGTTTCTTGTTGAACATGTCCT |
| | | | CACATTGATCAAACAATTTA |
| 920 | SLC39A6 | 3804214 | TCAGTCATCTGTCTTCTCAAAACATA |
| 921 | SLC39A6 | 3804217 | GTTTTATAGCCATTTCCATCATCAGTTTCCTGTCTCTGCT |
| | | | GGGGGTTATCTTAGTGCCTCTCATGAATCGGGTGTTTTT |
| | | | CAAATTTCTCCTGAGTTTCCTTGTGGCACTGGCCGTTGG |
| | | | GACTTTGAGTGGTGATGCTT |
| 922 | SLC39A6 | 3804218 | GAATGCAACAGAGTTCAACTATCTCTGTCCAGCCATCA |
| | | | TCAACCAAATTGATGCTAGATCTTGTCTGATTCATACAA |
| | | | GTGAAAAGAAGGCTGAAATCCCTCCAAAGACCTATTCA |
| | | | TTACA |
| 923 | SLC39A6 | 3804219 | GCATCAAAGCTACTGACATCTCATGGC |
| 924 | SLC39A6 | 3804221 | TGAGCCGGCTGGCTGGTAGGAAAACAAATGAATCTGTG |
| | | | AGTGAGCCCCGAAAAGGCTTTATGT |
| 925 | SLC39A6 | 3804222 | ACTCAGATAGTTCAGGTAAAGATCCTAGAAACAGCCAG |
| | | | GGGAAAGGAGCTCACCGACCAGAACATGCCAGTGGTA |
| | | | GAAGGAATGTCAAGGACAGTGTTAGTGCTAGTGAAGTG |
| | | | ACCTCAACTGTGTACAACACTGTCTCTGAAGGAACTCA |
| | | | CTTTCTAGAGACAATAGAGACTCCAAGACCTGGAAAAC |
| | | | TCTTCCCCAAAGATGTAAGCAGCTCCACTCCACCCAGT |
| | | | GTCACATCAAAGAGCCGG |
| 926 | SLC39A6 | 3804223 | CGCAATGGCGAGGAAGTTATCTGTAATCTTGATCCTGA |
| | | | CCTTTGCCCTCTCTGTCACAAATCCCCTTCATGAACTAA |
| | | | AAGCAGCTGCTTTCCCCCAGACCACTGAGAAAATTAGT |
| | | | CCGAATTGGGAATCTGGCATTAATGTTGACTTGGCAATT |
| | | | TCCACACGGCAATATCATCTACAACAGCTTTTCTACCGC |
| | | | TATGGAGA |
| 927 | SLC39A6 | 3804226 | TGTGGAACCAAACCTGCGCGCGTGGCCGGGCCGTGGGA |
| | | | CAACGAGGCCGCGGAGAC |
| 928 | SLC39A6 | 3804227 | AGATTTCTCGAAGACACCAGTGGGCCCG |
| 929 | SLC39A6 | 3804228 | CTGCGCGCGGCGGTAATTAGTGATTGTCTTCCAGCTTCG |
| | | | CGAAGGCTAGGGGCGCGGCTGCCGGGTGGCTGCGCGGC |
| | | | GCTGCCCCCGGACCGAGGGGCAGCCAACCCAATGAAAC |
| | | | CACCGCGTGTTCGCGCCTG |
| 930 | BCL2 | 3811353 | TGAAGAAAAATAAAGTACAGTGTGAG |
| 931 | BCL2 | 3811354 | TGTATTGAAAGCTTTTGTTATCAAGATTTTCATACTTTT |
| | | | ACCTTCCATGGCTCTTTTTAAGATTGATACTTTTAAGAG |
| | | | GTGGCTGATATTCTGCAACACTGTACACATAAAAAATA |
| | | | CGGTAAGGATACTTTACATGGTTAAGGTAAAGTAAGTC |
| | | | TCCAGTTGGCCACCATTAGCTATAATGGCACTTTGTTTG |
| | | | TGTTGTTGGAAAAAGTCACATTGCCATTAAACTTTCCTT |
| | | | GTCTGTC |
| 932 | BCL2 | 3811355 | CTGTAGTGTAGATACTGAGTAAATCCATGCACCTAAAC |
| | | | CTTTTGGAAAATCTGCCGTGGGCCCTCCAGATAGCTCAT |
| | | | TTCATTAAGTTTTTCCCTCCAAGGTAGAATTTGCA |
| 933 | BCL2 | 3811356 | GGAGGATGGAAAGGCTCGCTCAATCAAGAAAATTC |
| 934 | BCL2 | 3811357 | GACCTTGGACAATCATGAAATATGCATCTCACTGGATG |
| | | | CAAAGAAAATCAGATGGAGCATGAATGGTACTGTACCG |
| | | | GTTCATCTGGACTGCCCCAGAAAAATAACTTCAAGCAA |
| | | | ACATCCTATCAACAACAAGGTTGTTCTGCATACCAAGC |
| | | | TGAG |
| 935 | BCL2 | 3811358 | CCTGTGCTGCTATCCTGCCAAAATCATTTTAATGGAGTC |
| | | | AGTTTGCAGTATGCTCCACGTGGTAAGATCCTCCAAGCT |
| | | | GCTTTAGAAGTAACAATGAAGAACGTGGACGTTTTTAA |
| | | | TATAAAGCCTGTTTTGTCTTTTGTTGTTGTTCAAACGGG |
| | | | ATTCACAGAGTATTTGAAAAATGTATATATATTAAGAG |
| | | | GTCACGGGGGCTAATTGCTGGCTGGCTGCCTTTTGCTGT |
| | | | GGGGTTTTGTTACCTGGTTTTAATAACAGTAAATGTGCC |
| | | | CAGCCTCTTGGCCCCAGAACTGTACAGTATTGTGGCTGC |
| | | | ACTTGCTCTAAGAGTAGTTGATGTTGCATTTTCCTTATT |
| | | | GTTAAAAACATGTTAGAAGCAATGAATGTATATAAAAG |
| | | | CCTCAACTAGTCATTTTTTTCTCCTCTTCTTTTTTTTCATT |
| | | | ATATCTAATTATTTTGCAGTTGGGCAACAGAGAACCAT |
| | | | CCCTATTTTGTATTGAAGAGGGATTCACATCTGCATCTT |
| | | | AACTGCTCTTTATGAATGAAAAAACAGTCCTCTGTATGT |
| | | | ACTCCTCTTTACACTGGCCAGGGTCAGAGTTAAATAGA |
| | | | GTATATGCACTTTCCAAATTGGGGACAAGGGCTCTAAA |
| | | | AAAAGCCCCAAAAGGAGAAGAACATCTGAGAACCTCC |
| | | | TCGGCCCTCCCAGTCCCTCGCTGCACAAATACTCCGCAA |
| | | | GAGAGGCCAGAATGACAGCTGACAGGGTCTATGGCCAT |
| | | | CGGGTCGTCTCCGAAGATTTGGCAGGGGCAGAAAACTC |
| | | | TGGCAGGCTTAAGATTTGGAATAAAGTCACAGAATTAA |
| | | | GGAAGCACCTCAATTTAGTTCAAACAAGACGCCAACAT |
| | | | TCTCTCCACAGCTCACTTACCTCTCTGTGTTCAGATGTG |
| | | | GCCTTCCATTTATATGTGATCTTTGTTTTATTAGTAAATG |
| | | | CTTATCATCTAAAGATGTAGCTCTGGCCCAGTGGGAAA |
| | | | AATTAGGAAGTGATTATAAATCGAGAGGAGTTATAATA |
| | | | ATCAAGATTAAATGTAAATAATCAGGGCAATCCCAACA |
| | | | CATGTCTAGCTTTCACCTCCAGGATCTATTGAGTGAACA |
| | | | GAATTGCAAATAGTCTCTATTTGTAATTGAACTTATCCT |
| | | | AAAACAAATAGTTTATAAATGTGAACTTAAACTCTAAT |
| | | | TAATTCCAACTGTACTTTTAAGGCAGTGGCTGTTTTTAG |
| | | | ACTTTCTTATCACTTATAGTTAGTAATGTACACCTACTC |
| | | | TATCAGAGAAAAACAGGAAAGGCTCGAAATACAAGCC |
| | | | ATTCTAAGGAAATTAGGGAGTCAGTTGAAATTCTATTCT |
| | | | GATCTTATTCTGTGGTGTCTTTTGCAGCCCAGACAAATG |
| | | | TGGTTACACACTTTTTAAGAAATACAATTCTACATTGTC |
| | | | AAGCTTATGAAGGTTCCAATCAGATCTTTATTGTTATTC |
| | | | AATTTGGATCTTTCAGGGATTTTTTTTTTAAATTATTATG |
| | | | GGACAAAGGACATTTGTTGGAGGGGTGGGAGGGAGGA |
| | | | AGAATTTTTAAATGTAAAACATTCCCAAGTTTGGATCA |
| | | | GGGAGTTGGAAGTTTTCAGAATAACCAGAACTAAGGGT |
| | | | ATGAAGGACCTGTATTGGGGTCGATGTGATGCCTCTGC |
| | | | GAAGAACCTTGTGTGACAAATGAGAAACATTTTGAAGT |
| | | | TTGTGGTACGACCTTTAGATTCCAGAGACATCAGCATG |
| | | | GCTCAAAGTGCAGCTCCGTTTGGCAGTGCAATGGTATA |
| | | | AATTTCAAGCTGGATATGTCTAATGGGTATTTAAACAAT |
| | | | AAATGTGCAGTTTTAACTAACAGGATATTTAATGACAA |
| | | | CCTTCTGGTTGGTAGGGACATC |
| 936 | BCL2 | 3811359 | CGTCCCTGGGCAATTCCGCATTTAATTCATGGTATTCAG |
| | | | GATTACATGCATGTTTGGTTAAACCCATGAGATTCATTC |
| | | | AGTTAAAAATCCAGATGGCAAATGACCAGCAGATTCAA |
| | | | ATCTATGGTGGTTTGACCTTTAGAGAGTTGCTTTACGTG |
| | | | GCCTGTTTCAACACAGACCCACCCAGAGCCCTCCTGCC |
| | | | CTCCTTCCGCGGGGGCTTTCTCATGGCTGTCCTTCAGGG |
| | | | TCTTCCTGAAATGCAGTGGTGCTTACGCTCCACCAAGA |
| | | | AAGC |
| 937 | BCL2 | 3811360 | TAGGCCCGTTTTCACGTGGAGCATGGGAGCCACGACCC |
| | | | TTCTTAAGACATGTATCACTGTAGAGGGAAGGAACAGA |
| | | | GGCCCTGGGCCCTTCCTATCAGAAGGACATGGTGAAGG |
| | | | CTGGGAACGTGAGGAGAGGCAATGGCCACGGCCCATTT |
| | | | TGGCTGTAGCACATGGCACGTTGGCTGTGTGGCCTTGG |
| | | | CCCACCTGTGAGTTTAAAGCAAGGCTTTAAATGACTTTG |
| | | | GAGAGGGTCACAAATCCTAAAAGAAGCATTGAAGTGA |
| | | | GGTGTCATGGATTAATTGACCCCTGTCTATGGAATTACA |
| | | | TGTAAAACATTATCTTGTCACTGTAGTTTGGTTTTATTT |
| | | | GAAAACCTGACAAAAAAAAAGTTCCAGGTGTGGAATAT |
| | | | GGGGGTTATCTGTACATCCTGGGGCATT |
| 938 | BCL2 | 3811361 | CAACAGGGCAGTGTGGTCTCCGAATGTCTGGAAGCTGA |
| | | | T |
| 939 | BCL2 | 3811362 | CACCTGGATGTTCTGTGCCTGTAAACATAGATTCGCTTT |
| | | | CCATGTTGTTGGCCGGATCACCATCTGAAGAGCAGACG |
| | | | GATGGAAAAAGGACCTGATCATTGGGGAAGCTGGCTTT |
| | | | CTGGCTGCTGGAGGCTGGGGAGAAGGTGTTCATTCACT |
| | | | TGCATTTCTTTGCCCTGGGGGCTGTGATATTAACAGAGG |
| | | | GAGGGTTCCTGTGGGGGGAAGTCCATGCCTCCCTGGCC |
| | | | TGAAGAAGAGACTCTTTGCATATGACTCACATGATGCA |
| | | | TACCTGGTGGGAGGAAAAGAGTTGGGAACTTCAGATGG |
| | | | ACCTAGTACCCACTGAGATTTCCACGCCGAAGGACAGC |
| | | | GATGGGAAAAATGCCCTTAAATCATAGGAAAGTATTTT |
| | | | TTTAAGCTACCAATTGTGCCGAGAAAAGCATTTTAGCA |
| | | | ATTTATACAATATCATCCAGTACCTTAAGCCCTGATTGT |
| | | | GTATATTCATATATTTTGGATACGCACCCCCCAACTCCC |
| | | | AATACTGGCTCTGTCTGAGTAAGAAACAGAATCCTCTG |
| | | | GAACTTGAGGAAGTGAACATTTCGGTGACTTCCGCATC |
| | | | AGGAAGGCTAGAGTTACCCAGAGCATCAGGCCGCCACA |
| | | | AGTGCCTGCTTTTAGGAGACCGAAGTC |
| 940 | BCL2 | 3811363 | GTGGGAGCTTGCATCACCCTGGGTGCCTATCTGGGCCA |
| | | | CAA |
| 941 | BCL2 | 3811364 | TGTGGAACTGTACGGCCCCAGCATGCGGCCTCTGTTTG |
| | | | ATTTCTCCTGGCTGTCTCTGAAGACTCTGCTCAGTTTGG |
| | | | CCCT |
| 942 | BCL2 | 3811433 | GCAATACCATTCTCATGCCAGTGTACAAATTACATGAA |
| | | | AGAGCATCATTTTTCTAGTGTCTGAGGATTGGCTGCTTA |
| | | | TGGCCAATTTTGGCAGCAAGACGATAGGATTAAAAATA |
| | | | GCTTGAAGATGATCTAGTCTTAAATAATATATTTCATGA |
| | | | TGAACTTTCCTTGGGAAAGTGCATCTTTCTGCCTACAAG |
| | | | AATCACATGACCCCTTTCAATAATTTATGTAGTAGAGA |
| | | | AAAACACACTATTTCTCATAGAGTTTTCAGTCATGTGCT |
| | | | GTGGTGTGATTGTTTCTGGACATTCATAAAATTTTATAG |
| | | | TTAACTGAATTCTCTTTTCTGTTTTGTTGCTATTTAACGT |
| | | | CCATTGAAAACATGGCTTTCTTTTGCGCATTCTGTTACT |
| | | | TTCAGCTGTACTTTCTAATAAGAATGGATTGCCCTTTTT |
| | | | AGCAATCTTTGATTGAACTGGTACATTTCAGATTACTTA |
| | | | AATGTCATCAGGCCACACAGCATACCAGG |
| 943 | BCL2 | 3811441 | ACTTCGCCGAGATGTCCAGCCAGCTGCACCTGACGCCC |
| | | | TTCACCGCGCGGGGACGCTTTGCCACGGTGGTGGAGGA |
| | | | GCTCTTCAGGGACGGGGTGAACTGGGGGAGGATTGTGG |
| | | | CCTTCTTTGAGTTCGGTGGGGTCATGTGTGTGGAGAGCG |
| | | | TCAACCGGGAGATGTCGCCCCTGGTGGACAACATCGCC |
| | | | CTGTGGATGACTGAGTACC |
| 944 | BCL2 | 3811442 | GCCACCTGTGGTCCACCTGACCCTC |
| 945 | BCL2 | 3811443 | CCCGCACCGGGCATCTTCTCCTCCCAGCCCGGGCACAC |
| | | | GCCCCATCCAGCCGCATCCCGGGACCCGGTCGCCAGGA |
| | | | CCTCGCCGCT |
| 946 | BCL2 | 3811444 | GTACGATAACCGGGAGATAGTGATGAAGTACATCCATT |
| | | | ATAAGCTGTCGCAGAGGGGCTACGA |
| 947 | BCL2 | 3811445 | CACAGAGGAAGTAGACTGATATTAACAATACTTACTAA |
| | | | TAATAACGTGCCTCATGAAATAAAGATCCGAAAGGAAT |
| | | | TGGAATAAAAATTTCCTGCATCTCATGCCAAGGGGGAA |
| | | | ACACCAGAATCAAGTGTTCCGCGTGATTGAAGACACCC |
| | | | CCTCGTCCAAGAATGCAAAGCACATCCAATAAAATAGC |
| | | | TGGATTATAACTCCTCTTCTTT |
| 948 | BCL2 | 3811446 | TGTAACTTTCAATGGAAACCTTTGAGATTTTTTACTTAA |
| | | | AGTGCATTCGAGTAAATTTAATTTCCAGGCAGCTTAATA |
| | | | CATTCTTTTTAGCCGTGTTACTTGTAGTGTGTATGCCCT |
| | | | GCTTTCACTCAGTGTGTACAGGGAAACGCACCTGATT |
| 949 | BCL2 | 3811447 | GGCGCGTCCTGCCTTCATTTATCCAGCAGCTTTTCGGAA |
| | | | AATGCATTTGCTGTTCGGAGTTTAATCAGAAGAGGATT |
| | | | CCTGCCTCCGTCCCCGGCTCCTTCATCGTCCCCTCTCCC |
| | | | CTGTCTCTCTCCTGGGGAGGCGTGAAGCGGTCCCGTGG |
| | | | ATAGAGATTCATGCCTGTGCCCGCGCGTGTGTGCGCGC |
| | | | GTGTAAATTGCCGAGAAGGGGAAAACATCACAGGACTT |
| | | | CTGCGAATACCGGACTGAAAATTGTAATTCATCTGCCG |
| | | | CCGCCGCTGCCTTTTTTTTTTCTCGAGCTCTTGAGATCTC |
| | | | CGGTTGGGATTCCTGCGGATTGACATTTCTGTGAAGCA |
| | | | GAAGTCTGGGAATCGATCTGGAAATCCTCCTAATTTTTA |
| | | | CTCCCTCTCCCCGCGACTCCTGATTCATTGGGAA |
| 950 | BCL2 | 3811448 | GGAGGCGGCCGTAGCCAGCGCCGCCGC |
| 951 | BCL2 | 3811449 | TGCCGGGGCTCCGGGCCCTCCCTGCCGGCGGCCGTCAG |
| 952 | BCL2 | 3811450 | CCGCCGCTCTCCGTGGCCCCGCCGCGCTGCC |
| 953 | CCNE1 | 3828126 | TTTTAAATGTCCCGCTCTGAGCCGG |
| 954 | CCNE1 | 3828128 | GACGGCGGCGCGGAGTTCTCGGCTCGCTCCAGGAAGAG |
| | | | G |
| 955 | CCNE1 | 3828129 | GAAATGGCCAAAATCGACAGGACGGCGAGGGACCAGT |
| | | | GTGGGAGCC |
| 956 | CCNE1 | 3828130 | TCTAATGCAGCCACAGCCCATATGGCCCAGCACTGTAC |
| | | | CTGTCAGTGGGCACTGGCCTCTGCCAGTCCTGGGATTCC |
| | | | AGGAAGCTTGGTGTTCCTGACTGGCACCGTCTGAGATT |
| | | | ACAGATATGTGCCTAGCCTGGAAGA |
| 957 | CCNE1 | 3828131 | AGAAGATGATGACCGGGTTTACCCAAACTCAACGTGCA |
| | | | AGCCTCGGATTATTGCACCATCCAGAGGCT |
| 958 | CCNE1 | 3828132 | AAAGACATACTTAAGGGATCAGCACTTTCTTGAGCAAC |
| | | | ACCCTCTTCTGCAGCCAAAAATGCGAGCAATTCTTCTGG |
| | | | ATTGGT |
| 959 | CCNE1 | 3828133 | GGGAGACCTTTTACTTGGCACAAGATTTCTTTGACCGGT |
| | | | ATATGGCGACACAAGAAAATGTTGTAAAAACTCTTTTA |
| | | | CAGCTTATTGGGATTTCATCTTTAT |
| 960 | CCNE1 | 3828134 | GAAATCTATCCTCCAAAGTTGCACCAGTTTGCGTATGTG |
| | | | ACAGATGGAGCTTGTTCAGGAGATGAAATTCTCACCAT |
| | | | GGAATT |
| 961 | CCNE1 | 3828135 | TGGCGTTTAAGTCCCCTGACTATTGTGTCCTGGCTGAAT |
| | | | GTATACATGCAGGTTGCATATCTAAATGACTTACATGA |
| | | | AGTGCTACTGCCGCAGTATCCC |
| 962 | CCNE1 | 3828136 | CTGGATGTTGACTGCCTTGAATTTCCTTATGGTATACTT |
| | | | GCTGCTTCGGCCTTGTATCATTTCTCGTCATCTGAATTG |
| | | | ATGCAAAAGG |
| 963 | CCNE1 | 3828137 | CTGTGTCAAGTGGATGGTTCCATTTGCCATGGTTATAAG |
| | | | GGAGACGGGGAGCTCAAAACTGAAGCACTTCAGGGGC |
| | | | GTCGCTGATGAAGATGCACACAACATA |
| 964 | CCNE1 | 3828138 | GACAAAGCCCGAGCAAAGAAAGCCATGTTGTCTGAACA |
| | | | AAATAGGGCTTCTCCTC |
| 965 | CCNE1 | 3828139 | GTAAGAAGCAGAGCAGCGGGCCGGAAATGGCGTGA |
| 966 | CCNE1 | 3828140 | GCGTGCGTTTGCTTTTACAGATATCTGAATGGAAGAGT |
| | | | GTTTCTTCCACAACAGAAGTATTTCTGTGGATGGCATCA |
| | | | AACAGGGCAAAGTGTTTTTTATTGAATGCTTATAGGTTT |
| | | | TTTTTAAATAAGTGGGTCAAGTACACCAGCCACCTCCA |
| | | | GACACCAGTGCGTGCTCCCGATGCTGCTATGGAAGGTG |
| | | | CTACTTGACCTAAGGGACTCCCA |
| 967 | CCNE1 | 3828141 | TGGGCTCCGTTGTACCAAGTGGAGCAGGTGGTTGCGGG |
| | | | CAAGCGTTGTGCAGAGCCCATAGCCAGCTGGGCAGGGG |
| | | | GCTGCCCTCTCCACATTATCAGTTGACAGTGTACAATGC |
| | | | CTTTGATGAACTGTT |
| 968 | CCNE1 | 3828142 | GTAAGTGCTGCTATATCTATCCATTTTTTAATAAAG |
| 969 | MIA | 3833795 | TCTAGGTGGTGTGGGCGAAGTTTGGGACTGGTTTAGGG |
| | | | CGGGGACAAGACCAAGAACACAAGTTTCCTTGTACTAC |
| | | | GGGAGAGAGGGA |
| 970 | MIA | 3833796 | GGTCCCTGGTGTGCCTTGGTGTCATCATCTTGCTGTCTG |
| 971 | MIA | 3833798 | TTCAGGACTACATGGCCCCCGACTGCCGATTCCTGACC |
| | | | ATTCACCGGGGCCAAGTGGT |
| 972 | MIA | 3833799 | GCCGTGGGCGGCTCTTCTGGGGAGG |
| 973 | MIA | 3833800 | CATTTAAGCTGAGATTCATATGACAAGGATGGAGCAGT |
| | | | TATGTGGAGATCAGGGAGAAGGGAGAATGCAAAGGCC |
| | | | TTCAGCAGGCACAAGCTTGCCATCTTCCCAGACCCTAG |
| | | | CTTTTAACTCCTCTTCCCCAG |
| 974 | MIA | 3833801 | GTTCAGGGAGATTACTATGGAGATCTGGCTGCTCGCCT |
| | | | GGGCTATTTCCCCAGTAGCATTGTCCGAGAGGACCAGA |
| | | | CCC |
| 975 | MYBL2 | 3886226 | AAAGTGCTTCAACCCGCGCCGGCGGCGACTGCAGTTCC |
| | | | TGCGAGCGAGGAGCGCGGGACCTGCTGACACGCTGACG |
| | | | CCTTCGAGCGCGGCCC |
| 976 | MYBL2 | 3886229 | GCTGCACTACCAGGACACAGATTCAGATGTGCCGGAGC |
| | | | AGAGGGATAGCAAGTGCAAGGTCAAATGGACCCATGA |
| | | | GGA |
| 977 | MYBL2 | 3886231 | CCCTGGTGAGGCAGTTTGGACAGCAGGACTGGAAGTTC |
| | | | CTGGCCAGCCACTTCC |
| 978 | MYBL2 | 3886232 | TCCAGACCTTGTCAAGGGGCCATGGACCAAAGAGGA |
| 979 | MYBL2 | 3886235 | TGATTGCCAAGCACCTGAAGGGCCGGCTGGGGAAGCAG |
| | | | TGCCGTGAACGCTGGCACAACCACCTCAACCCTGAGGT |
| | | | GAAGAAGTCTTGCTGGACCGAGGAGGAGGACCGCATC |
| | | | ATCTGCGAGGCCCACAAGGTGCTGGGCAACCGCTGGGC |
| | | | CGAGATCGCCAAGATGTTGCCAGGG |
| 980 | MYBL2 | 3886237 | ACACAGGAGGCTTCTTGAGCGAGTCCAAAGACTGCAAG |
| | | | CCCC |
| 981 | MYBL2 | 3886238 | GCAGCCACCACATCGAAGGAACAGGAGCCCATCGGTAC |
| | | | AGATCTGGACGCAGTGCGAACACCAGAGCCCTTGGAGG |
| | | | AATTCCCGAAGCGTGAGGACCAGGAAGGCTCCCCACCA |
| | | | GAAACGAGCCTGCCTTACAAGTGGGTGGTGGAGGCAGC |
| | | | TAACCTCCTCATCCCTGCTGTGGGTTCTAGCCTCTCTGA |
| | | | AGC |
| 982 | MYBL2 | 3886239 | CTGATGCTTGGTGTGACCTGAGTAAATTTGACCTCCCTG |
| | | | AGGA |
| 983 | MYBL2 | 3886240 | GTATCAACAACAGCCTAGTGCAGCTGCAAGCGTCACAT |
| | | | CAGCAGCAAGTCCT |
| 984 | MYBL2 | 3886241 | CAGTGTGACCGAGTACCGCCTGGATGGCCACACCATCT |
| | | | CAGACCTGAGCCGGAGCAGCCGGGGCGAGCTGATCCCC |
| | | | ATCTCCCCCAGCACTGAAGTCGGGGGCTCTGGCATTGG |
| | | | CACACCGCCCTCTGTGCTCAAGCGGCAGAGGAAGAGGC |
| | | | GTGTGGCTCTGTCCCCTGTCACTGAGAATAGCACCAGTC |
| | | | TGTCCTTCCTGGATTCCTGTAACAGCCTCACGCCCAAGA |
| | | | GCA |
| 985 | MYBL2 | 3886242 | TTTCTGAACTTCTGGAACAAACAGGACACATTGGAGCT |
| | | | GGAGAGCCCCTCGCTGACATCCACCCCAGTGTGCAGCC |
| | | | AGAAGGTGGTGGTCACCACACCACTGCA |
| 986 | MYBL2 | 3886245 | CTCCATGGACAACACTCCCCACACGCCAACCCCGTTCA |
| | | | AGAACGCCCTGGAGAAGTACGGACCCCTGAAGCC |
| 987 | MYBL2 | 3886248 | ACTTGAAGGAGGTGCTGCGTTCTGAGGCTGGCATCGAA |
| | | | CTCATCATCGAGGACGACATCAGGCCCGAGAAGC |
| 988 | MYBL2 | 3886250 | CCGACAACTGCCCCTTCAAACTCTTCCAG |
| 989 | MYBL2 | 3886251 | CCTCACCCTGTCAGGTATCAAAGAAGACAAC |
| 990 | MYBL2 | 3886252 | CTTGCTCAACCAGGGCTTCTTGCAGGCCAAGCCCGAGA |
| | | | AGGCAGCAGTGGCCCAGAAGCCCCGAAGCCACTTCACG |
| | | | ACACCTGCCC |
| 991 | MYBL2 | 3886254 | CAGCCACACATCTCGGACCCTCATCTTGTCCTGA |
| 992 | MYBL2 | 3886255 | GTGTCACGAGCCCATTCTCATGTTTACAGGGGTTGTGGG |
| | | | GGCAGAGGGGGTCTGTGAATCTGAGAGTCATTCAGGTG |
| | | | ACCTCCTGCAGGGAGCCTTCTGCCACCAGCCCCTCCCCA |
| | | | GACTCTCAGGTGGAGGCAACAGGGCCATGTGCTGCCCT |
| | | | GTTGCCGAGCCCAGCTGTGGGCGGCTCCTGGTGCTAAC |
| | | | AACAAAGTTCCACTTCCAGGTCTGCCTGGTTCCCTCCCC |
| | | | AAGGCCACAGGGAGCTCCGTCAGCTTCTCCCAAG |
| 993 | UBE2C | 3887050 | GTCCTGCAGTTGCAGTCGTGTTCTCCGAGTTCCTGTCTC |
| | | | TCTGCCAACGCCGCC |
| 994 | UBE2C | 3887051 | TGGCTTCCCAAAACCGCGACCCAGCCGCCACTAGCGTC |
| | | | GCCGCCGCCCGTAAAGGAGCTGAGCCGA |
| 995 | UBE2C | 3887054 | GAGCTCAGACCGCTCTTTGAGACTCTCCCGAAGGAGAA |
| | | | TGGGAGGGTAGGGGCGCTGCCAGACTCCTTCCCTGGTG |
| | | | GGCCTAGATGAAGACGCTCAAGGACCCTCGTGACTTGG |
| | | | CCGAGACAGGGGAAGGGAGAAGTTGAGTCGGGCAAGG |
| | | | AAGAGATGCTAAAGCCTGGGGAATTAAGAACATGCCA |
| | | | GAATCATCCCGAGGGAGTCTGGAATTAGGGAGGGTGAG |
| | | | GACTCGCTAGGATCGTCCTGTGGATC |
| 996 | UBE2C | 3887059 | ATGTCTGGCGATAAAGGGATTTCTGCCTTCCCTGAATCA |
| | | | GACAACCTTTTCA |
| 997 | UBE2C | 3887061 | CAGTTTGTCTACTGTCCGGTCCCAG |
| 998 | UBE2C | 3887062 | ACTCAAGATTCTAGCAAGCCCCTTGTGTGGGGCT |
| 999 | UBE2C | 3887063 | GGTATAAGCTCTCGCTAGAGTTCCCCAGTGGCTACCCTT |
| | | | ACAATGCGCCCACAGTGAAGTTCCTCACGCCCTGCTAT |
| | | | CACCCCAACGTGGA |
| 1000 | UBE2C | 3887066 | ACACACATGCTGCCGAGCTCTGGAAAAACCCCA |
| 1001 | UBE2C | 3887067 | GTACCTGCAAGAAACCTACTCAAAGCAGGTCACCAGCC |
| | | | AGGAG |
| 1002 | UBE2C | 3887068 | GTGATTTCTGTATAGGACTCTTTATCTTGAGCTGTG |
| 1003 | MMP11 | 3939471 | GGCCTTTACGCGACATCCGAGCAGCGTGTCTATCCCAA |
| | | | AGGCCTAGGAGCATTTGCCCGGCTCGGTCAAATCTAGC |
| | | | GCAAGTTTGAAGCCTGCGGCCTCGCAATTTTAGCAGCTT |
| | | | C |
| 1004 | MMP11 | 3939472 | GTGGAGTCTTCTTGAATAAGCTGTGAAACATTTCCCCAC |
| | | | CCGCTTCCCTTTCTTGGCCCAGGCTTCCTGACCACAGCC |
| | | | TCACCTTTGAGCAGCTCAGAGCCCTGCCTGCCAGGATG |
| | | | CGAGCCACTGCCTGGATCGTGGCTCTGCAG |
| 1005 | MMP11 | 3939473 | GGCCCGGAGCGGCCCAGCAAGCCCAGCAGCCC |
| 1006 | MMP11 | 3939474 | GGCTCCGGCCGCCTGGCTCCGCAGCGCGGCCGCGCGCG |
| | | | CCCTCCTGCCCCCGA |
| 1007 | MMP11 | 3939475 | CATGCAGCCCTGCCCAGTAGCCCGGCACCTGCC |
| 1008 | MMP11 | 3939476 | GAAGCCCCCCGGCCTGCCAGCAGCCTCAG |
| 1009 | MMP11 | 3939477 | CTGTGGCGTGCCCGACCCATCTGATGGGCTGAGTGCCC |
| | | | GCAACCGACAGAAGAG |
| 1010 | MMP11 | 3939478 | GCGCTGGGAGAAGACGGACCTCACCTACAG |
| 1011 | MMP11 | 3939479 | CAGGGAGGTCATCTATGGGCAAACCCCCTGAAACCCCA |
| | | | ACTTAGACACATACACATATGGAGACCCTCCCTCAGCA |
| | | | GAGGGGCAGAGCCTCCGTCATCATGCAAAGAGTCGCAG |
| | | | CACATGCCTGCGGACGGGTGTTCAGTCACTCAGGCAGC |
| | | | CTTTACAAGAGACCTGTGAGGACCAGGCTCTGGGACTC |
| | | | CACGGTGAATGAGGCAGACACAGCCCCATCCTCTGTGT |
| | | | CAGTCTGAGGTGGGTGTCAGCCATGTCATTGTCCAACTC |
| | | | TACCATCACAACTTGGGCTTCGAGCAGGTGGAGACAGT |
| | | | GGTAAGCGGGGAGAGGCAATAGTGGGCATCTCACTGG |
| | | | GTGACCTGGGAGGACCCTGGGCAGGTGATGGGGAAGCT |
| | | | GAGGCTCACACATCCTGCGGGTGGGGACCCAGCCTGAA |
| | | | GAATGGGCTGGTGTCACACAGCATTGGAGCTGAGACTG |
| | | | GGGTCTTTAGAATTTCCTAGGTGGGGGCCTGGGAACCA |
| | | | ACAGGGGCTCAAGGAACCAAGGTGTCCCCACAGTAAGT |
| | | | GGCACTGTCAGGTCTAGGATGGGGGTCTCGGGACCCCT |
| | | | GGTCCTGGTTCTTTCCACTGAATTC |
| 1012 | MMP11 | 3939480 | TTCGGTTCCCATGGCAGTTGGTGCAGGAGCAGGTGCGG |
| | | | CAGACGATGGCAGAGGCCCTAAAGGTATGGAGCGATGT |
| | | | GACGCCACTCACCTTTACTGAGGTGCACGAGGGCCGTG |
| | | | CTGACATCATGATCGACTTCGCCA |
| 1013 | MMP11 | 3939481 | GGACGACCTGCCGTTTGATGGGCCTGGGGGCATCCTGG |
| | | | CCCATGCCTTCTTCCCCAAGACTCACCGAGAAGGGGAT |
| | | | GTCCACTTCGACTATGATGA |
| 1014 | MMP11 | 3939482 | AGGTGGCAGCCCATGAATTTGGCCA |
| 1015 | MMP11 | 3939483 | TACACCTTTCGCTACCCACTGAGTCTCAGCCCAGATGAC |
| | | | TGCAGGGGCGTTCAACACCTATATGGCCAGCCCTGGCC |
| | | | CACTGTCACCTCCAGGACCCCAGCCCTGGGCCCCCAGG |
| | | | CTGGGATAGACACCAATGA |
| 1016 | MMP11 | 3939484 | ACTGTGACTGCAGCATATGCCCTCAGCATGTGTC |
| 1017 | MMP11 | 3939485 | CCGAGGCGAGCTCTTTTTCTTCAAAGCGGGCTTTGTGTG |
| | | | GCGCCTCCGTGGGGGCCAGCTGCAGCCCGGCTACCCAG |
| | | | CATTGGCCTCTCGCCACTGGCAGGGACTGCCCAGCCCT |
| | | | GTGGACGCTGCCTTCGAGGATGCCCAGGG |
| 1018 | MMP11 | 3939486 | GGAGCATTGCAGATGCCAGGGACTTCACAAATGAAGGC |
| | | | ACAGCATGGGAAACCTGCGTGGGTTCCAGGGCAG |
| 1019 | MMP11 | 3939487 | TTTGTCACAGCCAAATGCCAGTGGAAGGAGCAGCCGCC |
| | | | CAGGCAGCCCTCTACTGATGAGAGTAACCTCACCCGTG |
| | | | CACTAGTTTACAGAGCATTCACTGCCCCAGCTTATCCCA |
| | | | GGCCTCCCGCTTCCCTCTGCGGGTGGGGTGCTGAGCAG |
| | | | GCATTATTGGCCTGCATGTTTTACTGA |
| 1020 | MMP11 | 3939488 | CTCAGTACTGGGTGTACGACGGTGAAAAGCCAGTCCTG |
| | | | GGCCCCGCACCCCTCACCGAGCTGGGCCTGGTGAGGTT |
| | | | CCCGGTCCATGCTGCCTTGGTCTGGGGTCCCGAGAAGA |
| | | | ACA |
| 1021 | MMP11 | 3939489 | AGCACCCGGCGTGTAGACAGTCCCGTGCCCCGCAGGGC |
| | | | CACTGACTGGAGAGGGGTGCCCTCTGAGATCGACGCTG |
| | | | CCTTCCAGGAT |
| 1022 | MMP11 | 3939491 | GCTATGCCTACTTCCTGCGCGGCCGCCTCTACTGGAAGT |
| | | | TTGACCCTGTGAAGGTG |
| 1023 | MMP11 | 3939492 | TCCTGACTTCTTTGGCTGTGCCGAGCCTGCCAACACTTT |
| | | | CC |
| 1024 | MMP11 | 3939493 | CTGCCAGGCCACGAATATCAGGCTAGAGACCCATGGCC |
| | | | ATCTTTGTGGCTGTGGGCACCAGGCATGGGACTGAGCC |
| | | | CATGTCTCCTCAGGGGGATGGGGTGGGGTACAACCACC |
| | | | ATGACAACTGCCGGGAGGGCCACGCAGGTCGTGGTCAC |
| | | | CTGCCAGCGACTGTCTCAGACTGGGCAGGGAGGCTTTG |
| | | | GCATGACTTAAGAGGA |
| 1025 | MMP11 | 3939494 | GAGTGTCCTTGCTGTATCCCTGTTGTGAGGTTCCTTCCA |
| | | | GGGGCTGGCACTGAAGCAAGGGTGCTGGGGCCCCATGG |
| | | | CCTTCAGCCCTGGCTGAGCAACTGGGCTGTAGGGCAGG |
| | | | GCC |
| 1026 | MMP11 | 3939495 | GCCTTCTGGCTGACAATCCTGGAAATCTGTTCTCCAGAA |
| | | | TCCAGGCCAAAAAGTTCACAGTCAAATGGGGAGGGGTA |
| | | | TTCTTCATGCAGGAGACCCCAGGCCCTGGAGGCTGCAA |
| | | | CATACCTCAATCCTGTCCCAGGCCGGATCCTCCTGAAGC |
| | | | CCTTTTCGCAGCACTG |
| 1027 | MDM2 | 3979479 | CTGATGGGTGTGCTAATTACACTGATTTAATCGATACCC |
| | | | ATTGTATGTGAAACAGTATATACACCATATTTACAATTA |
| | | | TGTATCAGTTTAACATTTAAAAAAACATTTCTAATATAA |
| | | | GTATCTCTCAAACTGTGGATTAACTTCTTGATTTATATT |
| | | | TAAATATGAATCTTGAGGAAAATAGTGAAAATAACCAT |
| | | | CTTGATTTAGTGTATTTCTCCCATATGTGAATTGTATAT |
| | | | AC |
| 1028 | RRM2 | 4006364 | AACGATGCCTTGTGTCAAGAAGAAGGCAGATTGGGCCT |
| | | | TGCGCTGGATTGGGGACAAAG |
| 1029 | RRM2 | 4006368 | AGCTGCAGCTCTCGCCGCTGAAGGGGC |

TABLE 9
Gene Targets
Gene
CDC20
KIF2C
PHGDH
NUF2
CENPF
EXO1
UBE2T
RRM2
MLPH
GPR160
CCNB1
CXXC5
PTTG1
FGFR4
FOXC1
ESR1
ANLN
BLVRA
EGFR
ACTR3B
NAT1
MYC
SFRP1
MELK
BAG1
CEP55
MKI67
TMEM45B
PGR
MDM2
KRT5
FOXA1
ORC6
CDH3
ERBB2
GRB7
CDC6
MAPT
BIRC5
KRT14
KRT17
TYMS
NDC80
SLC39A6
BCL2
CCNE1
MIA
MYBL2
UBE2C
MMP11

1-31. (canceled)

32. A method comprising:
a) obtaining or having obtained the presence or expression level in a biological sample from a subject having prostate cancer for targets CDC20, KIF2C, PHGDH, NUF2, CENPF, EXO1, UBE2T, RRM2, MLPH, GPR160, CCNB1, CXXC5, PTTG1, FGFR4, FOXC1, ESR1, ANLN, BLVRA, EGFR, ACTR3B, NAT1, MYC, SFRP1, MELK, BAG1, CEP55, MKI67, TMEM45B, PGR, MDM2, KRT5, FOXA1, ORC6, CDH3, ERBB2, GRB7, CDC6, MAPT, BIRC5, KRT14, KRT17, TYMS, NDC80, SLC39A6, BCL2, CCNE1, MIA, MYBL2, UBE2C, and MMP11;
b) subtyping the prostate cancer in the subject according to a genomic subtyping classifier based on the presence or expression levels of the plurality of targets, wherein said subtyping comprises assigning the prostate cancer to a luminal A subtype; and
c) administering an anti-cancer treatment that does not comprise androgen deprivation therapy to the subject who has the luminal A subtype, wherein the anti-cancer treatment that does not comprise androgen deprivation therapy is selected from the group consisting of surgery, chemotherapy, radiation therapy, immunotherapy, biological therapy, neoadjuvant chemotherapy, and photodynamic therapy.

33. (canceled)

34. A method comprising:
a) selecting a subject with prostate cancer having a luminal A subtype, wherein the subtype is determined according to a genomic subtyping classifier based on the presence or expression levels in a biological sample from the subject of targets CDC20, KIF2C, PHGDH, NUF2, CENPF, EXO1, UBE2T, RRM2, MLPH, GPR160, CCNB1, CXXC5, PTTG1, FGFR4, FOXC1, ESR1, ANLN, BLVRA, EGFR, ACTR3B, NAT1, MYC, SFRP1, MELK, BAG1, CEP55, MKI67, TMEM45B, PGR, MDM2, KRT5, FOXA1, ORC6, CDH3, ERBB2, GRB7, CDC6, MAPT, BIRC5, KRT14, KRT17, TYMS, NDC80, SLC39A6, BCL2, CCNE1, MIA, MYBL2, UBE2C, and MMP11; and
b) treating the subject with an anti-cancer treatment that does not comprise androgen deprivation therapy if the subject has the luminal A subtype, wherein the anti-cancer treatment that does not comprise androgen deprivation therapy is selected from the group consisting of surgery, chemotherapy, radiation therapy, immunotherapy, biological therapy, neoadjuvant chemotherapy, and photodynamic therapy.

35. The method of claim 34, wherein the biological sample is a biopsy.

36. The method of claim 34, wherein the biological sample is a urine sample, a blood sample or a prostate tumor sample.

37. The method of claim 36, wherein the blood sample is plasma, serum, or whole blood.

38. The method of claim 34, wherein the subject is a human.

39. The method of claim 34, wherein the levels of expression are increased or reduced compared to a control.

40. The method of claim 34, wherein the levels of expression are obtained by a method comprising in situ hybridization, a PCR-based method, an array-based method, sequencing, or an RNA assay method.

41. The method of claim 40, wherein the levels of expression are obtained by a method comprising using a reagent selected from the group consisting of a nucleic acid probe, or one or more nucleic acid primers.

42. The method of claim 41, wherein the levels of expression are obtained by a method comprising measuring the level of an RNA transcript.

43. The method of claim 32, wherein the biological sample is a biopsy.

44. The method of claim 32, wherein the biological sample is a urine sample, a blood sample or a prostate tumor sample.

45. The method of claim 44, wherein the blood sample is plasma, serum, or whole blood.

46. The method of claim 32, wherein the subject is a human.

47. The method of claim 32, wherein the levels of expression are increased or reduced compared to a control.

48. The method of claim 32, wherein the levels of expression are obtained by a method comprising in situ hybridization, a PCR-based method, an array-based method, sequencing, or an RNA assay method.

49. The method of claim 48, wherein the levels of expression are obtained by a method comprising using a reagent selected from the group consisting of a nucleic acid probe, or one or more nucleic acid primers.

50. The method of claim 49, wherein the levels of expression are obtained by a method comprising measuring the level of an RNA transcript.

51. The method of claim 32, wherein the surgery comprises cryotherapy.

52. The method of claim 32, wherein the radiation therapy comprises brachytherapy.

53. The method of claim 34, wherein the surgery comprises cryotherapy.

54. The method of claim 34, wherein the radiation therapy comprises brachytherapy.


SEQUENCE LISTING (1029 sequences)

SEQ ID NO: 1 - DNA, 102 bp, Homo sapiens
  source 1..102: mol_type = genomic DNA
gggtgctaggccggaaggggctgcagccgagggtggccctgattttgtggccggccaggagcgaaggggtccctttctgtcccctgagcaccgtcgcctcct

SEQ ID NO: 2 - DNA, 33 bp, Homo sapiens
  source 1..33: mol_type = genomic DNA
gcaccaactgcaaggacccctccccctgcgggc

SEQ ID NO: 3 - DNA, 146 bp, Homo sapiens
  source 1..146: mol_type = genomic DNA
ttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgc

SEQ ID NO: 4 - DNA, 144 bp, Homo sapiens
  source 1..144: mol_type = genomic DNA
gcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccacca

SEQ ID NO: 5 - DNA, 55 bp, Homo sapiens
  source 1..55: mol_type = genomic DNA
gtagaggaagccaagatccttcggctcagtggaaaaccacaaaatgcgccagagg

SEQ ID NO: 6 - DNA, 25 bp, Homo sapiens
  source 1..25: mol_type = genomic DNA
cgaagttcctggttcctggagggag

SEQ ID NO: 7 - DNA, 28 bp, Homo sapiens
  source 1..28: mol_type = genomic DNA
cctcttcctatctaagattgagggcaag

SEQ ID NO: 8 - DNA, 30 bp, Homo sapiens
  source 1..30: mol_type = genomic DNA
cagaacagactgaaagtactctacagccaa

SEQ ID NO: 9 - DNA, 35 bp, Homo sapiens
  source 1..35: mol_type = genomic DNA
cctgccagaccgtatcctggatgcgcctgaaatcc

SEQ ID NO: 10 - DNA, 145 bp, Homo sapiens
  source 1..145: mol_type = genomic DNA
acctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctg

SEQ ID NO: 11 - DNA, 74 bp, Homo sapiens
  source 1..74: mol_type = genomic DNA
cagcagcagaaacggcttcgaaatatgaccagtcactctgcccgagtgggctccctaagctggaacagctatat

SEQ ID NO: 12 - DNA, 115 bp, Homo sapiens
  source 1..115: mol_type = genomic DNA
catccaccaccatgatgttcgggtagcagaacaccatgtggccacactgagtggccacagccaggaagtgtgtgggctgcgctgggccccagatggacgacatttggccagtggt

SEQ ID NO: 13 - DNA, 94 bp, Homo sapiens
  source 1..94: mol_type = genomic DNA
gtaatgataacttggtcaatgtgtggcctagtgctcctggagagggtggctgggttcctctgcagacattcacccagcatcaaggggctgtcaa

SEQ ID NO: 14 - DNA, 31 bp, Homo sapiens
  source 1..31: mol_type = genomic DNA
gtgtccctggcagtccaatgtcctggcaaca

SEQ ID NO: 15 - DNA, 25 bp, Homo sapiens
  source 1..25: mol_type = genomic DNA
gcctgtctgagtgccgtggatgccc

SEQ ID NO: 16 - DNA, 25 bp, Homo sapiens
  source 1..25: mol_type = genomic DNA
ttatttggaagtacccaaccatggc

SEQ ID NO: 17 - DNA, 107 bp, Homo sapiens
  source 1..107: mol_type = genomic DNA
cacacatcccgggtcctgagtctgaccatgagcccagatggggccacagtggcatccgcagcagcagatgagaccctgaggctatggcgctgttttgagttggaccc

SEQ ID NO: 18 - DNA, 31 bp, Homo sapiens
  source 1..31: mol_type = genomic DNA
agaccaacccatcacctcagttgttttttat

SEQ ID NO: 19 - DNA, 75 bp, Homo sapiens
  source 1..75: mol_type = genomic DNA
tgcggcggtttacgcggcgttaagacttcgtagggttagcgaaattgaggtttcttggtattgcgcgtttctctt

SEQ ID NO: 20 - DNA, 25 bp, Homo sapiens
  source 1..25: mol_type = genomic DNA
gtccctaggtcaaggggactcgtga

SEQ ID NO: 21 - DNA, 31 bp, Homo sapiens
  source 1..31: mol_type = genomic DNA
gtttaattcacagtgccaatgtaaggactgt

SEQ ID NO: 22 - DNA, 275 bp, Homo sapiens
  source 1..275: mol_type = genomic DNA
tcttagggttaggtagcagctgtcaggaacttgcccctgcccataagatcctaaagggcccccatttgactctcaccagacagttagaacttgtttcctcctccgtgtcagccatcaagaggtgcttggggggctgtgcccagcaggacctcactgcccagcagatcagcaggggagccaagtggcctagatctgctgtggagtacccgactgtttgcctgcctgtctgccctcctcttcacctcattctcatcactgacgtctaccattggctt

SEQ ID NO: 23 - DNA, 72 bp, Homo sapiens
  source 1..72: mol_type = genomic DNA
atgatgtggctgcaataaacccagaactcttacagcttcttcccttacatccgaaggacaatctgcccttgc

SEQ ID NO: 24 - DNA, 32 bp, Homo sapiens
  source 1..32: mol_type = genomic DNA
caaaaacggagatccgtcaactccaaaattcc

SEQ ID NO: 25 - DNA, 94 bp, Homo sapiens
  source 1..94: mol_type = genomic DNA
ggacctatttcaccttgtacagaaacttggaggtttgcccctgaccaccctcgagatcgtgcagcactgactggctactgctctcggttctcca

SEQ ID NO: 26 - DNA, 30 bp, Homo sapiens
  source 1..30: mol_type = genomic DNA
ctgcccccactaggccttcctgccctgcag

SEQ ID NO: 27 - DNA, 64 bp, Homo sapiens
  source 1..64: mol_type = genomic DNA
ggctgaaataccattgaggatggtcagcgaggagatggaagagcaagtccattccatccgaggc

SEQ ID NO: 28 - DNA, 28 bp, Homo sapiens
  source 1..28: mol_type = genomic DNA
aggcccagaactctgaaatgagaatgaa

SEQ ID NO: 29 - DNA, 92 bp, Homo sapiens
  source 1..92: mol_type = genomic DNA
tatgacagtagttttccaaactgggaatttgcccgaatgattaaagaatttcgggctactttggaatgtcatccacttactatgactgatcc

SEQ ID NO: 30 - DNA, 26 bp, Homo sapiens
  source 1..26: mol_type = genomic DNA
gagcaccccctgaaatactctccttc

SEQ ID NO: 31 - DNA, 88 bp, Homo sapiens
  source 1..88: mol_type = genomic DNA
acatgaacccaagttgaaagtggacttaacaaagtatctggagaaccaagcattctgctttgactttgcatttgatgaaacagcttcg

SEQ ID NO: 32 - DNA, 33 bp, Homo sapiens
  source 1..33: mol_type = genomic DNA
aggccactggtacagacaatctttgaaggtgga

SEQ ID NO: 33 - DNA, 88 bp, Homo sapiens
  source 1..88: mol_type = genomic DNA
cgggacgtcttcctcctgaagaatcaaccctgctaccggaagttgggcctggaagtctatgtgacattcttcgagatctacaatggga

SEQ ID NO: 34 - DNA, 92 bp, Homo sapiens
  source 1..92: mol_type = genomic DNA
aggacggcaagcaacaggtgcaagtggtggggctgcaggagcatctggttaactctgctgatgatgtcatcaagatgatcgacatgggcagc

SEQ ID NO: 35 - DNA, 155 bp, Homo sapiens
  source 1..155: mol_type = genomic DNA
gctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctc

SEQ ID NO: 36 - DNA, 26 bp, Homo sapiens
  source 1..26: mol_type = genomic DNA
gctgggtgaggggcttttccagtcca

SEQ ID NO: 37 - DNA, 65 bp, Homo sapiens
  source 1..65: mol_type = genomic DNA
gcaaggttgccattccatccccttggagcctcaagcctcgaagcctgggcggtgccacattcctc

SEQ ID NO: 38 - DNA, 44 bp, Homo sapiens
  source 1..44: mol_type = genomic DNA
agtaccaagggggtgctgtgggatctgagacctccttgtttcct

SEQ ID NO: 39 - DNA, 40 bp, Homo sapiens
  source 1..40: mol_type = genomic DNA
gaagagatggaagcctgctctaacggggcgctgattccag

SEQ ID NO: 40 - DNA, 35 bp, Homo sapiens
  source 1..35: mol_type = genomic DNA
ccagatgtccagctttaacgaagccatgactcaga

SEQ ID NO: 41 - DNA, 96 bp, Homo sapiens
  source 1..96: mol_type = genomic DNA
aaggaccagactggcttgagctctctgagatgaccgagcagccagactatgacctggagacctttgtgaacaaagcggaatctgctctggcccagc

SEQ ID NO: 42 - DNA, 27 bp, Homo sapiens
  source 1..27: mol_type = genomic DNA
gtcatcaaggccttgcgcctggccatg

SEQ ID NO: 43 - DNA, 29 bp, Homo sapiens
  source 1..29: mol_type = genomic DNA
cgactgcaaataaaaatctgtttggtttg

SEQ ID NO: 44 - DNA, 29 bp, Homo sapiens
  source 1..29: mol_type = genomic DNA
gggacaggttctggtaaatgccaagtatg

SEQ ID NO: 45 - DNA, 245 bp, Homo sapiens
  source 1..245: mol_type = genomic DNA
ttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtctt

SEQ ID NO: 46 - DNA, 37 bp, Homo sapiens
  source 1..37: mol_type = genomic DNA
gtctccctagagatcctagaggatccctactgttttc

SEQ ID NO: 47 - DNA, 29 bp, Homo sapiens
  source 1..29: mol_type = genomic DNA
aggagccacatgctctcatcaagcagaaa

SEQ ID NO: 48 - DNA, 76 bp, Homo sapiens
  source 1..76: mol_type = genomic DNA
gtgcccaaccagtgaggccacgtttcgaaaagaagaaagaaacgacaaactaaaatacatgactgtgtagatgagg

SEQ ID NO: 49 - DNA, 84 bp, Homo sapiens
  source 1..84: mol_type = genomic DNA
gctggagaatactgcccagttactctagcgcgccaggccgaaccgcagcttcttggcttaggtacttctactcacagcggccga

SEQ ID NO: 50 - DNA, 67 bp, Homo sapiens
  source 1..67: mol_type = genomic DNA
atggcttttgcaaatctgcggaaagtgctcatcagtgacagcctggacccttgctgccggaagatct

SEQ ID NO: 51 - DNA, 67 bp, Homo sapiens
  source 1..67: mol_type = genomic DNA
gcagcctaagcatcattcctcttctcttcttagtggagataaaattacccactgctctccttacatt

SEQ ID NO: 52 - DNA, 1003 bp, Homo sapiens
  source 1..1003: mol_type = genomic DNA
gtgtgggcccttaccctagaagccaacttctcatgacctttctctatctccagaatccatgcagtgggaatgaaggtaaaagaaggttttcatgggatccagctgagagctctacggggaaaatggatctgaggagccatgtgctccatctcttttattttacaggtagagactaggggtatagagtgaggtgaattaccgcagtgacccacacattgttggcagacctaggattagaactctgtcttcctggttcccagcttggtgcttttgaaagcatacttgctgctttcttaccggcctggtgtctgccactttgggacagagtgtggacttgctcacctgccccatttcttagggattctcattctgtgtttgagcaagaatattcttattctggaaagaaccacataccacaggattctgggtgagcataaggaagattgtcttggggatctgacttagctcacgtatagtggctatgatgaattcagtgtcttattttttgcatatgtatatttttagtctaatattgcctgggtgtctgagcaagtctagatgaatttaattgctctcatttttcccctgcccctcttcctttggtctctcttttaggaaatgtttttctttcaacattcgtttcattcattatttactcattcggccaaccaacatttattgagtgccttccctgtatcagggacaggggcttacaaagtagaatttgatcccacctctgccctcagtagctcagtgtctaatggaggtagtgatgttcattaagcgtcgccagatactgtgctaggtgctgtgcctgttctctctcgcttgttcctcacacacttgagaaggccgaagctgattcatagcttggaaggcaggggccttggatttgaacccaggcctgaccaatggcagaacctatcagatgtgtggacagatgacattgcctttctttctttggatatatcaaaatcagccagcaggcaggaactcccatttt

SEQ ID NO: 53 - DNA, 28 bp, Homo sapiens
  source 1..28: mol_type = genomic DNA
tgtgtctgatggacatccaggctgcagg

SEQ ID NO: 54 - DNA, 86 bp, Homo sapiens
  source 1..86: mol_type = genomic DNA
atggtgctgtctagagagatgagccaggtgcccagagcccatgggccaatgctgccctttcttgagcatgccaaacaaagcggttg

SEQ ID NO: 55 - DNA, 50 bp, Homo sapiens
  source 1..50: mol_type = genomic DNA
cagtctcctccactctaagtaaaaatcagcatgagtcctagcccacattt

SEQ ID NO: 56 - DNA, 71 bp, Homo sapiens
  source 1..71: mol_type = genomic DNA
tgagtataccaaagatatctatgaactggcagtcatcagtgacttcctaaggttccggaaatgcatctctt

SEQ ID NO: 57 - DNA, 40 bp, Homo sapiens
  source 1..40: mol_type = genomic DNA
atgtgcctgcggctttacgagttctcacagaatgactttc

SEQ ID NO: 58 - DNA, 72 bp, Homo sapiens
  source 1..72: mol_type = genomic DNA
actgtgaaggccttattgttcgctctgccaccaaggtgaccgctgatgtcatcaacgcagctgagaaactcc

SEQ ID NO: 59 - DNA, 31 bp, Homo sapiens
  source 1..31: mol_type = genomic DNA
atctggaggccgcaacaaggaagggcatctt

SEQ ID NO: 60 - DNA, 113 bp, Homo sapiens
  source 1..113: mol_type = genomic DNA
tgagtgcggagactgaccacacctagggagaaaaaactcacttgagagaaagctgagtccattggaagggcttccaggaggatgcctggtctagggcctgcatggtcaacaca

SEQ ID NO: 61 - DNA, 122 bp, Homo sapiens
  source 1..122: mol_type = genomic DNA
attacttttcccatggcgagacctgctttccctcctgctggaggaggatctgggggaatttacctctgctctaactcctccctgcagtttccatctgagctctctggtattcactgatattc

SEQ ID NO: 62 - DNA, 45 bp, Homo sapiens
  source 1..45: mol_type = genomic DNA
gcagattccccaggcgacggcttcgatgaaggacggcaaatggga

SEQ ID NO: 63 - DNA, 33 bp, Homo sapiens
  source 1..33: mol_type = genomic DNA
gagaggtagctacccggatgcagtcctttggga

SEQ ID NO: 64 - DNA, 88 bp, Homo sapiens
  source 1..88: mol_type = genomic DNA
ttccttctccgtggcaccactacacatcaatattcctggcaatattcttcatcatggagacttcggcagcgacttcaaccagatgaaa

SEQ ID NO: 65 - DNA, 84 bp, Homo sapiens
  source 1..84: mol_type = genomic DNA
gaaacatggcttggatcattccgtctcccacctcagcccctccggagctgcctggacctcatcattccggagagtctaagtggc

SEQ ID NO: 66 - DNA, 125 bp, Homo sapiens
  source 1..125: mol_type = genomic DNA
cagtcccagcatcattgtgtggtcatgagaattaattaagctgatcatggtacttagtatatggtaaatagtacttagtatgtggaacatggtacttagtgtatggtaaattaactggagaatta

SEQ ID NO: 67 - DNA, 52 bp, Homo sapiens
  source 1..52: mol_type = genomic DNA
ggtatgaccccatcatttccccagaggtctcggcctcctttggtgttcagca

SEQ ID NO: 68 - DNA, 78 bp, Homo sapiens
  source 1..78: mol_type = genomic DNA
cctttgcccagtgcaagaagggggtgcgtgtggtgaactgtgcccgtggagggatcgtggacgaaggcgccctgctcc

SEQ ID NO: 69 - DNA, 78 bp, Homo sapiens
  source 1..78: mol_type = genomic DNA
gccttggtggaccatgagaatgtcatcagctgtccccacctgggtgccagcaccaaggaggctcagagccgctgtggg

SEQ ID NO: 70 - DNA, 54 bp, Homo sapiens
  source 1..54: mol_type = genomic DNA
atggttgactttacaagttatctcaataaaagtggccagatgcctaactcagaa

SEQ ID NO: 71 - DNA, 438 bp, Homo sapiens
  source 1..438: mol_type = genomic DNA
tggacttcgcatgcgttgatatttgaagcacgatcatcaaaactttgtgataattgatcgtagtgtttagtaacaatgtaaacacttaaaaaaattcaagatagaaaataaaaatgaaggcaagttgggactgccagagaagacccgtcactcctcatccaagttatctgcgactcccatatgttttgtgtcaaagactcacctttattgtgctgtccaatcccttccccagtgcagaaacaagtctcccatggagggggctggggcagacacagtttgctgaaaggagcaattttgagtggttgtggcattctgtgtccatttctggctccacagctttcttcatttgtaggaacaagtccttgtcctgttgttagtggctgatggaagttgtcacccaccaggcaccaaggcaggagtgaccctatactgtctttc

SEQ ID NO: 72 - DNA, 60 bp, Homo sapiens
  source 1..60: mol_type = genomic DNA
ccaagccttggattggtctggcagaagctctggggacactgatgcgagcctgggctgggt

SEQ ID NO: 73 - DNA, 96 bp, Homo sapiens
  source 1..96: mol_type = genomic DNA
aatgctgggaactgcctaagccccgcagtcattgtcggcctcctgaaagaggcttccaagcaggcggatgtgaacttggtgaacgctaagctgctg

SEQ ID NO: 74 - DNA, 47 bp, Homo sapiens
  source 1..47: mol_type = genomic DNA
tgggcttggtccaaggcactacgcctgtactgcaggggctcaatgga

SEQ ID NO: 75 - DNA, 75 bp, Homo sapiens
  source 1..75: mol_type = genomic DNA
caggcgtgcggctgctgtcctaccagacttcactggtgtcagatggggagacctggcacgtcatgggcatctcct

SEQ ID NO: 76 - DNA, 71 bp, Homo sapiens
  source 1..71: mol_type = genomic DNA
ccactgtgatcaatagggagagaaaatccacattcttgggctgaacgcgggcctctgacactgcttacact

SEQ ID NO: 77 - DNA, 114 bp, Homo sapiens
  source 1..114: mol_type = genomic DNA
tccagtaggaggcggcaagtttgaaaagtgatgacggttgacgtttgctgatttttgactttgcttgtagctgctccccgaactcgccgtcttcctgtcggcggccggcactgt

SEQ ID NO: 78 - DNA, 118 bp, Homo sapiens
  source 1..118: mol_type = genomic DNA
tgagcgcgagaggacggaggaaggaagcctgcagacagacgccttctccatcccaaggcgcgggcaggtgccgggacgctgggcctggcggtgttttcgtcgtgctcagcggtgggag

SEQ ID NO: 79 - DNA, 205 bp, Homo sapiens
  source 1..205: mol_type = genomic DNA
tcaaactatgtagttggaaagtgtcttcatctctcgttaatgaataaattgtaactgaaattgtacttcgaaagaatgatagaatttggatattggaggaggttccaaaaggaaatactggaagtttgggaaagttaggagactaacttggagcagaaatttcattcaattattaaagggtttagaagcctagcagaaaaatttg

SEQ ID NO: 80 - DNA, 91 bp, Homo sapiens
  source 1..91: mol_type = genomic DNA
aatgtagctgagattgtgattcatattcgcaataagatcttaacaggagctgatggtaaaaacctcaccaagaatgatctttatccaaatc

SEQ ID NO: 81 - DNA, 55 bp, Homo sapiens
  source 1..55: mol_type = genomic DNA
catgatctacatgagagccttacaaatagtatatggaattcgactggaacatttt

SEQ ID NO: 82 - DNA, 70 bp, Homo sapiens
  source 1..70: mol_type = genomic DNA
tgaactctgaagtcatgtatccacatttaatggaaggcttcttaccattcagcaatttagttactcatct

SEQ ID NO: 83 - DNA, 31 bp, Homo sapiens
  source 1..31: mol_type = genomic DNA
tttaagtggcattatcaactttattcacttc

SEQ ID NO: 84 - DNA, 61 bp, Homo sapiens
  source 1..61: mol_type = genomic DNA
tcctctgcggacaaaatgcaacagttaaacgccgcacaccaggaggcattaatgaaactgg

SEQ ID NO: 85 - DNA, 27 bp, Homo sapiens
  source 1..27: mol_type = genomic DNA
tgaggacaggtatttcattttagcctt

SEQ ID NO: 86 - DNA, 26 bp, Homo sapiens
  source 1..26: mol_type = genomic DNA
attcaggagctacaacaatcactaaa

SEQ ID NO: 87 - DNA, 30 bp, Homo sapiens
  source 1..30: mol_type = genomic DNA
aaaatgaaagatacggtccagaagcttaaa

SEQ ID NO: 88 - DNA, 117 bp, Homo sapiens
  source 1..117: mol_type = genomic DNA
atctatggagactcagttgactgcctgccttcatgtcagttggaagtgcagttatatcaaaagaaaatacaggacctttcagataatagggaaaaattagccagtatcttaaaggag

SEQ ID NO: 89 - DNA, 105 bp, Homo sapiens
  source 1..105: mol_type = genomic DNA
cgttcaaaagactgatgattgtgaagaaggaaaaacttgccacagcacaattcaaaataaataagaagcatgaagatgttaagcaatacaaacgcacagtaattg

SEQ ID NO: 90 - DNA, 45 bp, Homo sapiens
  source 1..45: mol_type = genomic DNA
aaaaagaggtgctgtctatgaacgagtaaccacaattaatcaaga

SEQ ID NO: 91 - DNA, 87 bp, Homo sapiens
  source 1..87: mol_type = genomic DNA
aaaactgctttggagaaataccacgacggtattgaaaaggcagcagaggactcctatgctaagatagatgagaagacagctgaactg

SEQ ID NO: 92 - DNA, 148 bp, Homo sapiens
  source 1..148: mol_type = genomic DNA
aaaagttgaagcgaatggaagtatcagaagtaccaaataatgttggcttcatcagtttttatacactctcataagtagttaataagatgaatttaatgtaggcttttattaatttataattaaaataacttgtgcagctattcatgtc

SEQ ID NO: 93 - DNA, 32 bp, Homo sapiens
  source 1..32: mol_type = genomic DNA
gctgcgggcagtttgaattagactctgggctc

SEQ ID NO: 94 - DNA, 36 bp, Homo sapiens
  source 1..36: mol_type = genomic DNA
cgcgccagaactgtactctccgagaggtcgttttcc

SEQ ID NO: 95 - DNA, 79 bp, Homo sapiens
  source 1..79: mol_type = genomic DNA
tgtcccagaccctactcggtcacggactcacactttaggggatcattttcttcctccgtaaaagaattggagatgacta

SEQ ID NO: 96 - DNA, 34 bp, Homo sapiens
  source 1..34: mol_type = genomic DNA
tttacaaatgttggagtaataaagaaggcagaac

SEQ ID NO: 97 - DNA, 133 bp, Homo sapiens
  source 1..133: mol_type = genomic DNA
gaaagaagggctgcctacaagagctcttcagaaaattcaagagcttgaaggacagcttgacaaactgaagaaggaaaagcagcaaaggcagtttcagcttgacagtctcgaggctgcgctgcagaagcaaaaa

SEQ ID NO: 98 - DNA, 78 bp, Homo sapiens
  source 1..78: mol_type = genomic DNA
gaagatttctcatgaacttcaagtcaaggagtcacaagtgaatttccaggaaggacaactgaattcaggcaaaaaaca

SEQ ID NO: 99 - DNA, 137 bp, Homo sapiens
  source 1..137: mol_type = genomic DNA
tattttgggatggtatttatagggatgatatttgtatgtattaatcagatgcgtttgtcttttcctttaacaatataattattatactttgcaattttttttcctggtagaataagtaatgattcggtctctgtacc

SEQ ID NO: 100 - DNA, 30 bp, Homo sapiens
  source 1..30: mol_type = genomic DNA
gaagccaacaagctgcgcagtctgcagatg

SEQ ID NO: 101 - DNA, 64 bp, Homo sapiens
  source 1..64: mol_type = genomic DNA
agactcttccacaagccaccatgaatcaccgcgacattgcccggcatcaggcttcatcatctgt

SEQ ID NO: 102 - DNA, 152 bp, Homo sapiens
  source 1..152: mol_type = genomic DNA
taggagagatttctctgcatcttacttttctggggaacaagaggtgactccaagtcgatcaactttgcaaatagggaaaagagatgctaatagcagtttctttgacaattctagcagtcctcatcttttggatcaattaaaagcgcagaatc

SEQ ID NO: 103 - DNA, 49 bp, Homo sapiens
  source 1..49: mol_type = genomic DNA
actgaaaaaattgacggaagatttgagttgtcagcgacaaaatgcagaa

SEQ ID NO: 104 - DNA, 118 bp, Homo sapiens
  source 1..118: mol_type = genomic DNA
gagctctcccgtcaacagcgttctttccaaacactggaccaggagtgcatccagatgaaggccagactcacccaggagttacagcaagccaagaatatgcacaacgtcctgcaggctg

SEQ ID NO: 105 - DNA, 60 bp, Homo sapiens
  source 1..60: mol_type = genomic DNA
agagtttaagcaaaagttgtgcagagctgaacaggcgttccaggcgagtcagatcaagga

SEQ ID NO: 106 - DNA, 84 bp, Homo sapiens
  source 1..84: mol_type = genomic DNA
aaaggccagagaagtctgccacctggaggcagaactcaagaacatcaaacagtgtttaaatcagagccagaattttgcagaaga

SEQ ID NO: 107 - DNA, 501 bp, Homo sapiens
  source 1..501: mol_type = genomic DNA
gccttgctgagtgctttagagttaaaaaagaaagaatatgaagaattgaaagaagagaaaactctgttttcttgttggaaaagtgaaaacgaaaaacttttaactcagatggaatcagaaaaggaaaacttgcagagtaaaattaatcacttggaaacttgtctgaagacacagcaaataaaaagtcatgaatacaacgagagagtaagaacgctggagatggacagagaaaacctaagtgtcgagatcagaaaccttcacaacgtgttagacagtaagtcagtggaggtagagacccagaaactagcttatatggagctacagcagaaagctgagttctcagatcagaaacatcagaaggaaatagaaaatatgtgtttgaagacttctcagcttactgggcaagttgaagatctagaacacaagcttcagttactgtcaaatgaaataatggacaaagaccggtgttaccaagacttgcatgccgaatatgagagcctc

SEQ ID NO: 108 - DNA, 767 bp, Homo sapiens
  source 1..767: mol_type = genomic DNA
tttggcttttgatcagcagcctgccatgcatcattcctttgcaaatataattggagaacaaggaagcatgccttcagagaggagtgaatgtcgtttagaagcagaccaaagtccgaaaaattctgccatcctacaaaatagagttgattcacttgaattttcattagagtctcaaaaacagatgaactcagacctgcaaaagcagtgtgaagagttggtgcaaatcaaaggagaaatagaagaaaatctcatgaaagcagaacagatgcatcaaagttttgtggctgaaacaagtcagcgcattagtaagttacaggaagacacttctgctcaccagaatgttgttgctgaaaccttaagtgcccttgagaacaaggaaaaagagctgcaacttttaaatgataaggtagaaactgagcaggcagagattcaagaattaaaaaagagcaaccatctacttgaagactctctaaaggagctacaacttttatccgaaaccctaagcttggagaagaaagaaatgagttccatcatttctctaaataaaagggaaattgaagagctgacccaagagaatgggactcttaaggaaattaatgcatccttaaatcaagagaagatgaacttaatccagaaaagtgagagttttgcaaactatatagatgaaagggagaaaagcatttcagagttatctgatcagtacaagcaagaaaaacttattttactacaaagatgtgaagaaaccggaaatgcatatgaggatctta

SEQ ID NO: 109 - DNA, 83 bp, Homo sapiens
  source 1..83: mol_type = genomic DNA
actgtgaaatagatgcggaagaaaagtatatttcagggcctcatgagttgtcaacaagtcaaaacgacaatgcacaccttcag

SEQ ID NO: 110 - DNA, 135 bp, Homo sapiens
  source 1..135: mol_type = genomic DNA
tctgcaaacaacaatgaacaagctgaatgagctagagaaaatatgtgaaatactgcaggctgaaaagtatgaactcgtaactgagctgaatgattcaaggtcagaatgtatcacagcaactaggaaaatggcaga

SEQ ID NO: 111 - DNA, 321 bp, Homo sapiens
  source 1..321: mol_type = genomic DNA
tggctccattggacgagagtaattcctacgagcacttgacattgtcagacaaagaagttcaaatgcactttgccgaattgcaagagaaattcttatctttacaaagtgaacacaaaattttacatgatcagcactgtcagatgagctctaaaatgtcagagctgcagacctatgttgactcattaaaggccgaaaatttggtcttgtcaacgaatctgagaaactttcaaggtgacttggtgaaggagatgcagctgggcttggaggaggggctcgttccatccctgtcatcctcttgtgtgcctgacagctctagtctta

SEQ ID NO: 112 - DNA, 143 bp, Homo sapiens
  source 1..143: mol_type = genomic DNA
gctgacaagcgtgactctggagatggagtccaagttggcggcagaaaagaaacagacggaacaactgtcacttgagctggaagtagcacgactccagctacaaggtctggacttaagttctcggtctttgcttggcatcgaca

SEQ ID NO: 113 - DNA, 392 bp, Homo sapiens
  source 1..392: mol_type = genomic DNA
agataccaattatgagcctccaggggaagataaaacccagggctcttcagaatgcatttctgaattgtcattttctggtcctaatgctttggtacctatggatttcctggggaatcaggaagatatccataatcttcaactgcgggtaaaagagacatcaaatgagaatttgagattacttcatgtgatagaggaccgtgacagaaaagttgaaagtttgctaaatgaaatgaaagaattagactcaaaactccatttacaggaggtacaactaatgaccaaaattgaagcatgcatagaattggaaaaaatagttggggaacttaagaaagaaaactcagatttaagtgaaaaattggaatatttttcttgtgatcaccaggagttactcc

SEQ ID NO: 114 - DNA, 792 bp, Homo sapiens
  source 1..792: mol_type = genomic DNA
attgagcatgaagccctctacctggaggctgacttagaggtagttcaaacagagaagctatgtttagaaaaagacaatgaaaataagcagaaggttattgtctgccttgaagaagaactctcagtggtcacaagtgagagaaaccagcttcgtggagaattagatactatgtcaaaaaaaaccacggcactggatcagttgtctgaaaaaatgaaggagaaaacacaagagcttgagtctcatcaaagtgagtgtctccattgcattcaggtggcagaggcagaggtgaaggaaaagacggaactccttcagactttgtcctctgatgtgagtgagctgttaaaagacaaaactcatctccaggaaaagctgcagagtttggaaaaggactcacaggcactgtctttgacaaaatgtgagctggaaaaccaaattgcacaactgaataaagagaaagaattgcttgtcaaggaatctgaaagcctgcaggccagactgagtgaatcagattatgaaaagctgaatgtctccaaggccttggaggccgcactggtggagaaaggtgagttcgcattgaggctgagctcaacacaggaggaagtgcatcagctgagaagaggcatcgagaaactgagagttcgcattgaggccgatgaaaagaagcagctgcacatcgcagagaaactgaaagaacgcgagcgggagaatgattcacttaaggataaagttgagaaccttgaaagggaattgcagatgtcagaagaaaaccaggagctagtgattcttgatgcc

SEQ ID NO: 115 - DNA, 296 bp, Homo sapiens
  source 1..296: mol_type = genomic DNA
agagtctagacccaccaatagaggaagagcatcagctgagaaatagcattgaaaagctgagagcccgcctagaagctgatgaaaagaagcagctctgtgtcttacaacaactgaaggaaagtgagcatcatgcagatttacttaagggtagagtggagaaccttgaaagagagctagagatagccaggacaaaccaagagcatgcagctcttgaggcagagaattccaaaggagaggtagagaccctaaaagcaaaaatagaagggatgacccaaagtctgagaggtctggaatta

SEQ ID NO: 116 - DNA, 286 bp, Homo sapiens
  source 1..286: mol_type = genomic DNA
gctcaatgagagagtggcagccctgcataatgaccaagaagcctgtaaggccaaagagcagaatcttagtagtcaagtagagtgtcttgaacttgagaaggctcagttgctacaaggccttgatgaggccaaaaataattatattgttttgcaatcttcagtgaatggcctcattcaagaagtagaagatggcaagcagaaactggagaagaaggatgaagaaatcagtagactgaaaaatcaaattcaagaccaagagcagcttgtctctaaactgtcccaggtg

SEQ ID NO: 117 - DNA, 29 bp, Homo sapiens
  source 1..29: mol_type = genomic DNA
agagaaaaataggctagctggagagttgc

SEQ ID NO: 118 - DNA, 108 bp, Homo sapiens
  source 1..108: mol_type = genomic DNA
atagtgaattgaagaagagcctagattgcatgcacaaagaccaggtggaaaaggaagggaaagtgagagaggaaatagctgaatatcagctacggcttcatgaagctg

SEQ ID NO: 119 - DNA, 123 bp, Homo sapiens
  source 1..123: mol_type = genomic DNA
aatccagacataccgagagaaattgacttctaaagaagaatgtctcagttcacagaagctggagatagaccttttaaagtctagtaaagaagagctcaataattcattgaaagctactactca

SEQ ID NO: 120 - DNA, 175 bp, Homo sapiens
  source 1..175: mol_type = genomic DNA
tcttcctttggaaattcatgatgccatatcagatggttttagaattctgcactttaataatgtaatgcatatgccatatataatatcccagagggatctgctataatattgcataatcgaattcatatttctgcagcaaaatgtgtggatactctcacaaggcaggataaataga

SEQ ID NO: 121 - DNA, 30 bp, Homo sapiens
  source 1..30: mol_type = genomic DNA
tttttccatatgcttataaaaagaaattca

SEQ ID NO: 122 - DNA, 25 bp, Homo sapiens
  source 1..25: mol_type = genomic DNA
atggacaatctaaaatatgtaaatc

SEQ ID NO: 123 - DNA, 93 bp, Homo sapiens
  source 1..93: mol_type = genomic DNA
gaagttgttgatcaaatcctgtaaacagctggaagaggaaaaggagatactgcagaaagaactctctcaacttcaagctgcacaggagaagca

SEQ ID NO: 124 - DNA, 226 bp, Homo sapiens
  source 1..226: mol_type = genomic DNA
tctaaacaagattcccgagggtctcctttgctaggtccagttgttccaggaccatctccaatcccttctgttactgaaaagaggttatcatctggccaaaataaagcttcaggcaagaggcaaagatccagtggaatatgggagaatggtagaggaccaacacctgctaccccagagagcttttctaaaaaaagcaagaaagcagtcatgagtggtattcaccctg

SEQ ID NO: 125 - DNA, 137 bp, Homo sapiens
  source 1..137: mol_type = genomic DNA
gactagcccatatatcctgcgaagaacaaccatggcaactcggaccagcccccgcctggctgcacagaagttagcgctatccccactgagtctcggcaaagaaaatcttgcagagtcctccaaaccaacagctggtg

SEQ ID NO: 126 - DNA, 179 bp, Homo sapiens
  source 1..179: mol_type = genomic DNA
ctcagcggagcccagtagattcaggcaccatcctccgagaacccaccacgaaatccgtcccagtcaataatcttcctgagagaagtccgactgacagccccagagagggcctgagggtcaagcgaggccgacttgtccccagccccaaagctggactggagtccaacggcagtgagaac

SEQ ID NO: 127 - DNA, 149 bp, Homo sapiens
  source 1..149: mol_type = genomic DNA
ttctctttagtcagggcatgctttattagtgaggagaaaacaattccttagaagtcttaaatatattgtactctttagatctcccatgtgtaggtattgaaaaagtttggaagcactgatcacctgttagcattgccattcctctactg

SEQ ID NO: 128 - DNA, 118 bp, Homo sapiens
  source 1..118: mol_type = genomic DNA
cttcctagaggtgtgctataccatgcgtctgtcgttgtgcttttttctgtttttagaccaattttttacagttctttggtaagcattgtcgtatctggtgatggattaacatatagcc

SEQ ID NO: 129 - DNA, 34 bp, Homo sapiens
  source 1..34: mol_type = genomic DNA
ggccatcagcgccagtgccactcgcgccctcaag

SEQ ID NO: 130 - DNA, 47 bp, Homo sapiens
  source 1..47: mol_type = genomic DNA
tcggagcgggtttctccaaccgcaatcggctccgctcaaggggagga

SEQ ID NO: 131 - DNA, 204 bp, Homo sapiens
  source 1..204: mol_type = genomic DNA
aaacgtgtcgtctggaatgggcttgggggccacgcctgcacatctccgcgagacagagggataaagtgaagatggtgctgttattgttacctcgagtgccacatgcgacctctgagatatgtacacagtcattcttactatcgcactcagccattcttactacgctaaagaagaaataattattcgaggatatttgcctggccc

SEQ ID NO: 132 - DNA, 96 bp, Homo sapiens
  source 1..96: mol_type = genomic DNA
tagtgaatcccagtcactgagtggagttgagagtctaagaacctctgaaatttgagaactgctggaccagagcctttagagctctgataaggtgtc

SEQ ID NO: 133 - DNA, 159 bp, Homo sapiens
  source 1..159: mol_type = genomic DNA
tggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgata

SEQ ID NO: 134 - DNA, 58 bp, Homo sapiens
  source 1..58: mol_type = genomic DNA
ctatctcatgggatcaagcctattctcgtatttgatggatgtactttaccttctaaaa

SEQ ID NO: 135 - DNA, 83 bp, Homo sapiens
  source 1..83: mol_type = genomic DNA
agacgacaagccaatcttcttaagggaaagcaacttcttcgtgaggggaaagtctcggaagctcgagagtgtttcacccggtc

SEQ ID NO: 136 - DNA, 106 bp, Homo sapiens
  source 1..106: mol_type = genomic DNA
ctcgtggctccctatgaagctgatgcgcagttggcctatcttaacaaagcgggaattgtgcaagccataattacagaggactcggatctcctagcttttggctgta

SEQ ID NO: 137 - DNA, 124 bp, Homo sapiens
  source 1..124: mol_type = genomic DNA
tgaaattgatcaagctcggctaggaatgtgcagacagcttggggatgtattcacggaagagaagtttcgttacatgtgtattctttcaggttgtgactacctgtcatcactgcgtgggattgga

SEQ ID NO: 138 - DNA, 151 bp, Homo sapiens
  source 1..151: mol_type = genomic DNA
tatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctggg

SEQ ID NO: 139 - DNA, 87 bp, Homo sapiens
  source 1..87: mol_type = genomic DNA
tgatgattccatagctcttcaaatagcacttggaaataaagatataaatacttttgaacagatcgatgactacaatccagacactgc

SEQ ID NO: 140 - DNA, 144 bp, Homo sapiens
  source 1..144: mol_type = genomic DNA
catagttgggatgacaaaacatgtcaaaagtcagctaatgttagcagcatttggcataggaattactctcccagaccagagtcgggtactgtttcagatgccccacaattgaaggaaaatccaagtactgtgggagtggaacga

SEQ ID NO: 141 - DNA, 153 bp, Homo sapiens
  source 1..153: mol_type = genomic DNA
acctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacga

SEQ ID NO: 142 - DNA, 26 bp, Homo sapiens
  source 1..26: mol_type = genomic DNA
gtggtgcagttgtggttccagggacc

SEQ ID NO: 143 - DNA, 466 bp, Homo sapiens
  source 1..466: mol_type = genomic DNA
gcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaa

SEQ ID NO: 144 - DNA, 69 bp, Homo sapiens
  source 1..69: mol_type = genomic DNA
tctgattgcaatattaagttacttgacagtcaaagtgaccagacctccaagctacgtttatctcatttc

SEQ ID NO: 145 - DNA, 140 bp, Homo sapiens
  source 1..140: mol_type = genomic DNA
ggctatataagtccagttctgcagactctctttctacaaccaagatcaaacctctaggacctgccagagccagtgggctgagcaagaagccggcaagcatccagaagagaaagcatcataatgccgagaacaagccgggg

SEQ ID NO: 146 - DNA, 86 bp, Homo sapiens
  source 1..86: mol_type = genomic DNA
agagataacatccaactaactccagaagcggaagaggatatatttaacaaacctgaatgtggccgtgttcaaagagcaatattcca

SEQ ID NO: 147 - DNA, 28 bp, Homo sapiens
  source 1..28: mol_type = genomic DNA
tctcgctgtgtcacaatctcagctcact

SEQ ID NO: 148 - DNA, 124 bp, Homo sapiens
  source 1..124: mol_type = genomic DNA
ttcctccatcctaggcagaaataaagtcccaaatctttgttttttaacgggtcatagaggacccatcatcaccctttattcattccttgatcatctcaggctagagaagtctagggatacagct

SEQ ID NO: 149 - DNA, 65 bp, Homo sapiens
  source 1..65: mol_type = genomic DNA
tgtcctggttcatcttagttaatgtgttctttgccaaggtgatctaagttgcctaccttgaattt

SEQ ID NO: 150 - DNA, 75 bp, Homo sapiens
  source 1..75: mol_type = genomic DNA
accagaggctggtgactccagagtacacaactcaacacagaaaaggaaggccagtcagctagtaggcatagaaaa

SEQ ID NO: 151 - DNA, 34 bp, Homo sapiens
  source 1..34: mol_type = genomic DNA
atttaaatataataagccagccttcctcaagaat

SEQ ID NO: 152 - DNA, 29 bp, Homo sapiens
  source 1..29: mol_type = genomic DNA
gctctaaccactgcaaatcatgtttttct

SEQ ID NO: 153 - DNA, 42 bp, Homo sapiens
  source 1..42: mol_type = genomic DNA
ctctgcaacacatatcctaccttgtctataccgctaactctc

SEQ ID NO: 154 - DNA, 86 bp, Homo sapiens
  source 1..86: mol_type = genomic DNA
catccctcaacatcgcaactgtgttgacctctattcagctgctcatgtcagaacccaaccctgatgacccgctcatggctgacata

SEQ ID NO: 155 - DNA, 90 bp, Homo sapiens
  source 1..90: mol_type = genomic DNA
tgaacctcctcagatccgatttctcactccaatttatcatccaaacattgattctgctggaaggatttgtctggatgttctcaaattgcc

SEQ ID NO: 156 - DNA, 58 bp, Homo sapiens
  source 1..58: mol_type = genomic DNA
tggagccaacacaccttatgagaaaggtgtttttaagctagaagttatcattcctgag

SEQ ID NO: 157 - DNA, 103 bp, Homo sapiens
  source 1..103: mol_type = genomic DNA
agagcttcacgtctgaagagagagctgcacatgttagccacagagccacccccaggcatcacatgttggcaagataaagaccaaatggatgacctgcgagctc

SEQ ID NO: 158 - DNA, 27 bp, Homo sapiens
  source 1..27: mol_type = genomic DNA
gtgtgtggttccttctacttggggatc

SEQ ID NO: 159 - DNA, 247 bp, Homo sapiens
  source 1..247: mol_type = genomic DNA
tcccgcgctgcgcttgaaaatcgcgcgcggccccgcggccagcctgggtaggggcaaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccact

SEQ ID NO: 160 - DNA, 27 bp, Homo sapiens
  source 1..27: mol_type = genomic DNA
agctgcagctctcgccgctgaaggggc

SEQ ID NO: 161 - DNA, 44 bp, Homo sapiens
  source 1..44: mol_type = genomic DNA
gacccgcgtcctggccagcaagaccgcgaggaggatcttccagg

SEQ ID NO: 162 - DNA, 125 bp, Homo sapiens
  source 1..125: mol_type = genomic DNA
tctgctgcgacccacggagtgcgacgggacagccacgttttcacatcgggccccgtgaaattgccgccaatggaaaggacttggtccagaaaaacgttagtttcatatggttcgcccggtactta

SEQ ID NO: 163 - DNA, 33 bp, Homo sapiens
  source 1..33: mol_type = genomic DNA
gctgcccccggcgtggaggatgagccgctgctg

SEQ ID NO: 164 - DNA, 86 bp, Homo sapiens
  source 1..86: mol_type = genomic DNA
cccgccgctttgtcatcttccccatcgagtaccatgatatctggcagatgtataagaaggcagaggcttccttttggaccgccgag

SEQ ID NO: 165 - DNA, 30 bp, Homo sapiens
  source 1..30: mol_type = genomic DNA
atcggaggaccccagaagacccctgcaggg

SEQ ID NO: 166 - DNA, 26 bp, Homo sapiens
  source 1..26: mol_type = genomic DNA
cccgaggagagatattttatatccca

SEQ ID NO: 167 - DNA, 204 bp, Homo sapiens
  source 1..204: mol_type = genomic DNA
tgacgatctgaggtcgaactagttcgctttcctcgtcttgtatgtttttccatgctgagtgcatctgtgtgtgtaagctgggttttatattacatggcatttcctgttttgtaacactttgcagttctttcttatggtattttcccgactctagagaagctgagacaatattaagtggtagcaatgtgatgactctttgtggcc

SEQ ID NO: 168 - DNA, 41 bp, Homo sapiens
  source 1..41: mol_type = genomic DNA
agtcttcttattgacacttacataaaagatcccaaagaaag

SEQ ID NO: 169 - DNA, 282 bp, Homo sapiens
  source 1..282: mol_type = genomic DNA
ccctgctgtactggactatgttttactgtctgtagaccctgaagctcaatatgaactacagaatacccaaacttgtattaatgtaaatcaagtgttgaggtttttaaaagaacactggagggaaaaactgaccagtaaaaataaaacatttcggtgtgagttcttcctttaggaagaggattggcaaatacttgaatttggcctttgtcccagagctcttatctagcagttggtaatcggaggtcttttactgtaatgcttcaattgctgataccgtatgtg

SEQ ID NO: 170 - DNA, 59 bp, Homo sapiens
  source 1..59: mol_type = genomic DNA
aacgatgccttgtgtcaagaagaaggcagactgggccttgcgctggattggggacaaag

SEQ ID NO: 171 - DNA, 28 bp, Homo sapiens
  source 1..28: mol_type = genomic DNA
tcgatattctggctcaagaaacgaggac

SEQ ID NO: 172 - DNA, 68 bp, Homo sapiens
  source 1..68: mol_type = genomic DNA
caactcgggcatgctcttgtgttcactgacggggacctgagatgctagatggcatatatccacattta

SEQ ID NO: 173 - DNA, 28 bp, Homo sapiens
  source 1..28: mol_type = genomic DNA
acacctggtacacaaaccatcggaggag

SEQ ID NO: 174 - DNA, 81 bp, Homo sapiens
  source 1..81: mol_type = genomic DNA
gctcattgggatgaattgcactctaatgaagcaatacattgagtttgtggcagacagacttatgctggaactgggttttag

SEQ ID NO: 175 - DNA, 135 bp, Homo sapiens
  source 1..135: mol_type = genomic DNA
agtagagaacccatttgactttatggagaatatttcactggaaggaaagactaacttctttgagaagagagtaggcgagtatcagaggatgggagtgatgtcaagtccaacagagaattcttttaccttggatgc

SEQ ID NO: 176 - DNA, 168 bp, Homo sapiens
  source 1..168: mol_type = genomic DNA
tgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggta

SEQ ID NO: 177 - DNA, 89 bp, Homo sapiens
  source 1..89: mol_type = genomic DNA
ctttagtgagcttagcacagcgggattaaacagtcctttaaccagcacagccagttaaaagatgcagcctcactgcttcaacgcagatt

SEQ ID NO: 178 - DNA, 1311 bp, Homo sapiens
  source 1..1311: mol_type = genomic DNA
agtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcatacttcagtcacccagtgtcttattctggcattgtctaaatctgagcattgtctagggggatcttaaactttagtaggaaaccatgagctgttaatacagtttccattcaaatattaatttcagaatgaaacataatttttttttttttttttgagatggagtctcgctctgttgcccaggctggagtgcagtggcgcgattttggctcactgtaacctccatctcctgggttcaagcaattctcctgtctcagcctccctagtagctgggactgcaggtatgtgctaccacacctggctaatttttgtatttttagtagagatggagtttcaccatattggtcaggctggtcttgaactcctgacctcaggtgatccacccacctcggcctcccaaagtgctgggattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctga

SEQ ID NO: 179 - DNA, 43 bp, Homo sapiens
  source 1..43: mol_type = genomic DNA
gtctcaaaattgaataatgcacaagtcttaagtgattaaaata

SEQ ID NO: 180 - DNA, 171 bp, Homo sapiens
  source 1..171: mol_type = genomic DNA
tgagcacccaaaggccggccctagagtccaggagaagagcgcagcggcgcggagctcccaggcgttccccgcagcgcgtcctcggtcctggaaccaccgcgccgcgcgtcctggcttccacatctgccccatttgcccgcggatcttgactttttcttggcgggcaaggcc

SEQ ID NO: 181 - DNA, 119 bp, Homo sapiens
  source 1..119: mol_type = genomic DNA
gagagccaggcgctaaccagccgctctgcgccccgcgccctgcttgcccccattatccagccttgccccggcgccctgacctgacgccctggcctgacgccctgcttcgtcgcctcctt

SEQ ID NO: 182 - DNA, 34 bp, Homo sapiens
  source 1..34: mol_type = genomic DNA
tgctggaccagggactgagcgtcccccggagagg

SEQ ID NO: 183 - DNA, 36 bp, Homo sapiens
  source 1..36: mol_type = genomic DNA
ttggaagttgttcaacgagattttgacctccgaagg

SEQ ID NO: 184 - DNA, 308 bp, Homo sapiens
  source 1..308: mol_type = genomic DNA
caaacctggggcgttagctcaactcttgcccccctgctgaaggagaccaaaacaatgcttgatcaggaaacaccatctggctttgcccccaggattctgtgactgccctggggagggcgcagtgacctgccaaccaaaattggtacaattgtaaacagccacagaaatgcttaaatgcaatatcatttctatgaaattaacgtgtttccattccattccagccaccaaaattgcccgtttgagctcagccctcaaaacaaagatgcctgtgtggctttgcccaacgttgggtcactgttttctgcata

SEQ ID NO: 185 - DNA, 30 bp, Homo sapiens
  source 1..30: mol_type = genomic DNA
atgcccaatatatttcttgtttctgatatt

SEQ ID NO: 186 - DNA, 32 bp, Homo sapiens
  source 1..32: mol_type = genomic DNA
cgttgaagggcaagattaagaaggaaagctcc

SEQ ID NO: 187 - DNA, 148 bp, Homo sapiens
  source 1..148: mol_type = genomic DNA
aagagggagctgctttccgacactgcccatctgaacgagacccactgcgcccgctgcctgcagccctaccagctgcttgtgaatagcaaaaggcagtgcctggaatgtggcctcttcacctgcaaaagctgtggccgcgtccacccgg

SEQ ID NO: 188 - DNA, 90 bp, Homo sapiens
  source 1..90: mol_type = genomic DNA
agtcgtgaagatcggctcactggagtggtactatgagcatgtgaaagcccgcttcaagaggttcggaagtgccaaggtcatccggtccct

SEQ ID NO: 189 - DNA, 50 bp, Homo sapiens
  source 1..50: mol_type = genomic DNA
agtggagagtaagaacggctttttgttcccaggcattttaggaatattaa

SEQ ID NO: 190 - DNA, 272 bp, Homo sapiens
  source 1..272: mol_type = genomic DNA
atgggtgggtaggtgaatacatggatggatgagccactgattgagtgggtggatgggtggatgaatagatgggtggaggatagataggtgggtgtatgggtgggtggatggattgatgcatggatggatgggctgcccattgagtaggtggatgagtggataaatgggtgggtgggtaggtgaatagatgaatagattgataaatagggggatgggtggattggtagatgggtagatggagggatacattgctgtgtggataggtgggtgaa

SEQ ID NO: 191 - DNA, 27 bp, Homo sapiens
  source 1..27: mol_type = genomic DNA
ggatgggtggatgggctgacaaatggc

SEQ ID NO: 192 - DNA, 82 bp, Homo sapiens
  source 1..82: mol_type = genomic DNA
ctgggcctgaactgatatctgaagagagaagtggagacagcgaccagacagatgaggatggagaacctggctcagaggccca

SEQ ID NO: 193 - DNA, 61 bp, Homo sapiens
  source 1..61: mol_type = genomic DNA
aaaaagcgcctcctctccgtccacgacttcgacttcgagggagactcagatgactccactc

SEQ ID NO: 194 - DNA, 69 bp, Homo sapiens
  source 1..69: mol_type = genomic DNA
tctgggtgccactcccatccggaagagcagccgaccagcatctcaccttccagacacggcgccctggct

SEQ ID NO: 195 - DNA, 34 bp, Homo sapiens
  source 1..34: mol_type = genomic DNA
ggtcgaatgtcatcaggaatgagcagctgcccct

SEQ ID NO: 196 - DNA, 69 bp, Homo sapiens
  source 1..69: mol_type = genomic DNA
ccgatgtggacacctctgatgaggaaagcatccgggctcacgtgatggcctcccaccattccaagcgga

SEQ ID NO: 197 - DNA, 80 bp, Homo sapiens
  source 1..80: mol_type = genomic DNA
atctttgagctgaataagcatatttcagctgtggaatgcctgctgacctacctggagaacacagttgtgcctcccttggc

SEQ ID NO: 198 - DNA, 186 bp, Homo sapiens
  source 1..186: mol_type = genomic DNA
gtcattcccgatctttccaccgagggcctctgtgatttgggggctttgtcaggaaagtggagcctcacggaaaagcatactggctaaaacacgcggcttcttcatcgactcaatctaatcatccccttggtgttcgtctgtgagaccccaggcagccagccctgtcgatctgtctcaataggcttc

SEQ ID NO: 199 - DNA, 30 bp, Homo sapiens
  source 1..30: mol_type = genomic DNA
gtgctggagtgcgcacggaggccgatgtag

SEQ ID NO: 200 - DNA, 48 bp, Homo sapiens
  source 1..48: mol_type = genomic DNA
aggaggaggccctgaggaggaagctggaggagctgaccagcaacgtca

SEQ ID NO: 201 - DNA, 78 bp, Homo sapiens
  source 1..78: mol_type = genomic DNA
caggagacctcgtccgaggaggaggaagccaaggacgaaaaggcagagcccaacagggacaaatcagttgggcctctc

SEQ ID NO: 202 - DNA, 30 bp, Homo sapiens
  source 1..30: mol_type = genomic DNA
ggcacggctgcccatcaaaccaacagacag

SEQ ID NO: 203 - DNA, 88 bp, Homo sapiens
  source 1..88: mol_type = genomic DNA
ctggggaccccgtccagtacaacaggaccacagatgaggagctgtcagagctggaggacagagtggcagtgacggcctcagaagtcca

SEQ ID NO: 204 - DNA, 62 bp, Homo sapiens
  source 1..62: mol_type = genomic DNA
attgaatccaggattgcagccctgagggccgcagggctcacggtgaagccctcgggaaagcc

SEQ ID NO: 205 - DNA, 129 bp, Homo sapiens
  source 1..129: mol_type = genomic DNA
tctttatgaggggactctgagcctctgctctgaggatctgaaacacacacaccctgacagtgtaaaatccaaaaggagccgcctgaatcatgttgcctcatgtggaaatccttagtccgccgccacgtg

SEQ ID NO: 206 - DNA, 288 bp, Homo sapiens
  source 1..288: mol_type = genomic DNA
aagaccacacaccagtgcagtgtgatgggcctttctgctgcttcattagtgtgaggatttccagggccacagtgaggaagaatgttaatgccagtgccagagcaaaggagaaagaagttggcaaaactgttgatttgcatgacagctgaaatgtaaatactttttaaaaaatatgtgatgtggaagcttcttaaaaggggatatgtccatttttttctaccttttaaatttctgaggaggccaaggcacttgtttgggctaagtatgtgattgataaagcaccatccc

SEQ ID NO: 207 - DNA, 58 bp, Homo sapiens
  source 1..58: mol_type = genomic DNA
atgatgattcttttgatcggaaatcagtgtaccgaggctcgctgacacagagaaaccc

SEQ ID NO: 208 - DNA, 99 bp, Homo sapiens
  source 1..99: mol_type = genomic DNA
aggagtggccccaaccaatgtgatcagtcgcaggaggcaaccaatcagaggctgaagggaagttacaaagttacacatgaagacttggccgatgaccag

SEQ ID NO: 209 - DNA, 25 bp, Homo sapiens
  source 1..25: mol_type = genomic DNA
aaacctgtggtggcccaccagtcct

SEQ ID NO: 210 - DNA, 77 bp, Homo sapiens
  source 1..77: mol_type = genomic DNA
acagagcagccctgcactgttttccctccaccacagccatcctgtccctcattggctctgtgctttccactatacac

SEQ ID NO: 211 - DNA, 1156 bp, Homo sapiens
  source 1..1156: mol_type = genomic DNA
cacctgcaagtggacagcgacattcagtcctgcactgctcacctgggtttactgatgactcctggctgccccaccatcctctctgatctgtgagaaacagctaagctgctgtgacttccctttaggacaatgttgtgtaaatctttgaaggacacaccgaagacctttatactgtgatcttttacccctttcactcttggctttcttatgttgctttcatgaatggaatggaaaaaagatgactcagttaaggcaccagccatatgtgtattcttgatggtctatatcggggtgtgagcagatgtttgcgtatttcttgtgggtgtgactggatattagacatccggacaagtgactgaactaatgatctgctgaataatgaaggaggaatagacaccccagtccccaccctacgtgcacccgctctgcaagttcccatgtgatctgtagaccaggggaaattacactgcggtcaagggcagagcctgcacatgacagcaagtgagcatttgatagatgctcagatgctagtgcagagagcctgctgggagacgaagagacagcaggcagagctccagatgggcaaggaagaggcttggttctagcctggctctgcccctcactgcagtggatccagtggggcagaggacagagggtcacaaccaatgagggatgtctgccaaggatgggggtgcagaggccacaggagtcagcttgccactcgcccattggttacatagatgatctctcagacaggctgggactcagagttatttcctagtatcggtgtgccccatccagttttaagtggagccctccaagactctccagagctgcctttgaacatcctaacagtaatcacatctcaccctccctgaggttcactttagacaggacccaatggctgcactgcctttgtcagagggggtgctgagaggagtggcttcttttagaatcaaacagtagagacaagagtcaagccttgtgtcttcaagcattgaccaagttaagtgtttccttccctctctcaataagacacttccaggagctttccaatctctcacttaaaactaaggtttgaatctcaaagtgttgctgggaggctgatactcctgcaacttcaggagacctgtgagcacacattagcagctgtttc

SEQ ID NO: 212 - DNA, 71 bp, Homo sapiens
  source 1..71: mol_type = genomic DNA
ccaggagtggagcccggacgcccgagccttcctgcttcgggatggggattaccgcggagccttagcaactg

SEQ ID NO: 213 - DNA, 71 bp, Homo sapiens
  source 1..71: mol_type = genomic DNA
atcctggctggctcaaaattccctctagattacctgcgaccacccccaggaacccggagactgaaactcct

SEQ ID NO: 214 - DNA, 98 bp, Homo sapiens
  source 1..98: mol_type = genomic DNA
cctgcagtccggagacgaacgcacggaccgggcctccggaggcaggttcggctggaaggaaccgctctcgcttcgtcctacacttgcgcaaatgtctc

SEQ ID NO: 215 - DNA, 112 bp, Homo sapiens
  source 1..112: mol_type = genomic DNA
gctttgtgcagtggccatttcatagccagtgaagtttatctgaggcacttgctaattgaaaacttttctcaataccctgccatgatgaaatatggttggcactggcaatttt

SEQ ID NO: 216 - DNA, 114 bp, Homo sapiens
  source 1..114: mol_type = genomic DNA
atgtccaagtcccgtgcggcggaggcagcagcgggggtgacagcgacggccccgagcccgcagatagtggagcagaggggtccagggaggcgctgcaccgacgttggggagaat

SEQ ID NO: 217 - DNA, 32 bp, Homo sapiens
  source 1..32: mol_type = genomic DNA
ttcaggaggagaactccgttacacgtcacgaa

SEQ ID NO: 218 - DNA, 376 bp, Homo sapiens
  source 1..376: mol_type = genomic DNA
aaaacctccggcctcgtcttgtgattccaccaatgcagccatcgccaagcaagccttgaaatagcccatcaagggcaaacaggcccccgaaaaaaagctcaaggaaaagtcgcaaactcacggatttctaccctgtcccaaggagctccaggaagggcaaagcggagctgcaatttgaagaaagggaaagaatagatgaattggttgaaagcggaaaggaaagaaggagtgaagactgacctcatcaatgacaaaggccgggtgtgaacaccagagggagctctcccggggcgcctttgtggtagaaaagcaccggagcctcacggagatcaccgacgccacgggacccctccatggtggatctccgtggatctcc

SEQ ID NO: 219 - DNA, 29 bp, Homo sapiens
  source 1..29: mol_type = genomic DNA
gttgaacgttttgtgcttcccatgcatgc

SEQ ID NO: 220 - DNA, 242 bp, Homo sapiens
  source 1..242: mol_type = genomic DNA
tgctccacttatcgggctcaccaaatacagctgcagtatgaattccatcttctacacaacagtaaaccagaatgttcatgttcacagcgttcatctcccatcctgttgtctaacgcacatggtttttttaaacttttctaagattgcatgagattctgcaacacaactgattataaaaacacttgaagtttttacctttttttttactttccaactctcgtgaatgtacagaggactttcca

SEQ ID NO: 221 - DNA, 296 bp, Homo sapiens
  source 1..296: mol_type = genomic DNA
caggggagaataagctgaacgcagctgttctctgaccttgaggcagagggcaaggagtaggtacaaggacgtgtaggagaatttatcttaaataggcttgttcacttgtgttgtccagaaacgacttttgatcatcagcgcgcatgactgctccctgaaaggaagaacaataatgttaattacccgcagactgtgtttgctccaggctttcggcattatgtctgtactgaataaaagcaagcagctccagctgttcgaggctgctctcttcttcagccattagtgccgggcagccc

SEQ ID NO: 222 - DNA, 107 bp, Homo sapiens
  source 1..107: mol_type = genomic DNA
agcttactcacatagcatattggtatatcaaaatgaaatgcaaggaaccaaaaataacataattgaaggcagtaaaagtgaaattaaataggaagatcatcagtcaa

SEQ ID NO: 223 - DNA, 132 bp, Homo sapiens
  source 1..132: mol_type = genomic DNA
gagcttcaggaaaagacttaatctgaaggatcctgcagctaaaaagctttgaaaactgtgttaaggggccccataagcatcgcttctaaacttcactgacaaaagggactggggtcatgctgtctggagtca

SEQ ID NO: 224 - DNA, 25 bp, Homo sapiens
  source 1..25: mol_type = genomic DNA
tgtatttcagcaggtcttcttgaaa

SEQ ID NO: 225 - DNA, 308 bp, Homo sapiens
  source 1..308: mol_type = genomic DNA
tccagttttcctgacagcttgtatagattattgcctgaatttctctaaaacaaccaagctttcatttaagtgtcaaaaattattttatttctttacagtaattttaatttggatttcagtccttgcttatgttttgggagacccagccatctaccaaagcctgaaggcacagaatgcttattctcgtcactgtcctttctatgtcagcattcagagttactggctgtcatttttcatggtgatgattttatttgtagctttcataacctgttgggaagaagttactactttggtacaggctatcagga

SEQ ID NO: 226 - DNA, 76 bp, Homo sapiens
  source 1..76: mol_type = genomic DNA
tgttttacttaaagttcagattccagcatatattgagatgaatattccctggttatactttgtcaatagttttctc

SEQ ID NO: 227 - DNA, 108 bp, Homo sapiens
  source 1..108: mol_type = genomic DNA
aaagacattggattacctttggatccatttgtcaactggaagtgctgcttcattccacttacaattcctaatcttgagcaaattgaaaagcctatatcaataatgatt

SEQ ID NO: 228 - DNA, 60 bp, Homo sapiens
  source 1..60: mol_type = genomic DNA
gttacagctgtcataagatcataattttatgaacagaaagaactcaggacatattaaaaa

SEQ ID NO: 229 - DNA, 256 bp, Homo sapiens
  source 1..256: mol_type = genomic DNA
ccctgactgatagcatttcagaatgtgtcttttgaagggctatgataccagttattaaatagtgttttattttaaaaacaaaataattccaagaagtttttatagttattcagggacactatattacaaatattactttgttattaacacaaaaagtgataagagttaacatttggctatactgatgtttgtgttactcaaaaaaactactggatgcaaactgttatgtaaatctgagatttcactgacaacttta

SEQ ID NO: 230 - DNA, 25 bp, Homo sapiens
  source 1..25: mol_type = genomic DNA
gtgcggggtttaaatctgaggctag

SEQ ID NO: 231 - DNA, 42 bp, Homo sapiens
  source 1..42: mol_type = genomic DNA
ctcttctcggcgtgctgcggcggaacggctgttggtttctgc

SEQ ID NO: 232 - DNA, 44 bp, Homo sapiens
  source 1..44: mol_type = genomic DNA
gtaggtccttggctggtcgggcctccggtgttctgcttctcccc

SEQ ID NO: 233 - DNA, 151 bp, Homo sapiens
  source 1..151: mol_type = genomic DNA
agccgcttcggactgcgaactaacgcggccttcttagctgctgcctgctctccctgcctcgcctgcgggagcctcccgagcgggagagggccgcaggagcgatttggggaggaaggtgggaggggactcaccaagagagcgccgaggtggg

SEQ ID NO: 234 - DNA, 125 bp, Homo sapiens
  source 1..125: mol_type = genomic DNA
tgctgaaaataaggcgaagatcaacatggcaggcgcaaagcgcgttcctacggcccctgctgcaacctccaagcccggactgaggccaagaacagctcttggggacattggtaacaaagtcagtg

SEQ ID NO: 235 - DNA, 43 bp, Homo sapiens
  source 1..43: mol_type = genomic DNA
aagcaaaaccttcagctactggaaaagtcattgataaaaaact

SEQ ID NO: 236 - DNA, 108 bp, Homo sapiens
  source 1..108: mol_type = genomic DNA
actggaaacatgagagccatcctaattgactggctagtacaggttcaaatgaaattcaggttgttgcaggagaccatgtacatgactgtctccattattgatcggttc

SEQ ID NO: 237 - DNA, 126 bp, Homo sapiens
  source 1..126: mol_type = genomic DNA
tgtgactgacaacacttatactaagcaccaaatcagacagatggaaatgaagattctaagagctttaaactttggtctgggtcggcctctacctttgcacttccttcggagagcatctaagattgg

SEQ ID NO: 238 - DNA, 68 bp, Homo sapiens
  source 1..68: mol_type = genomic DNA
tgatgtcgagcaacatactttggccaaatacctgatggaactaactatgttggactatgacatggtgc

SEQ ID NO: 239 - DNA, 93 bp, Homo sapiens
  source 1..93: mol_type = genomic DNA
aacattacctgtcatatactgaagaatctcttcttccagttatgcagcacctggctaagaatgtagtcatggtaaatcaaggacttacaaagc

SEQ ID NO: 240 - DNA, 75 bp, Homo sapiens
  source 1..75: mol_type = genomic DNA
tcaagaacaagtatgccacatcgaagcatgctaagatcagcactctaccacagctgaattctgcactagttcaag

SEQ ID NO: 241 - DNA, 29 bp, Homo sapiens
  source 1..29: mol_type = genomic DNA
aaacttgagttggagtactatatttacaa

SEQ ID NO: 242 - DNA, 35 bp, Homo sapiens
  source 1..35: mol_type = genomic DNA
attactgttgcatttacttttaataaagcttgtgg

SEQ ID NO: 243 - DNA, 158 bp, Homo sapiens
  source 1..158: mol_type = genomic DNA
cctggggatccaattgatgtatatgtttatatactgggttcttgttttatatacctggcttttactttattaatatgagttactgaaggtgatggaggtatttgaaaattttacttccataggacatactgcatgtaagccaagtcatggagaatctg

SEQ ID NO: 244 - DNA, 293 bp, Homo sapiens
  source 1..293: mol_type = genomic DNA
cccgggcagcgttcatagctcctgcccgggcgggcgcgcggcggcggcggcagaggcggctgagcctgagcggggatgtagaggcggcggcagcagaggcggcactggcggcaagagcagacgcccgagccgagcgagaagagcggcagagccttatcccctgaagccgggccccgcgtcccagccctgcccagcccgcgcccagccatgcgcgccgcctgctgagtccgggcgccgcacgctgagccctccgcccgcgagccgcgctcagctcgggggtgattagttgcttt

SEQ ID NO: 245 - DNA, 53 bp, Homo sapiens
  source 1..53: mol_type = genomic DNA
agttcgctgcaagtcggcggaaagtttggctgcgcgggttcccccgaagttca

SEQ ID NO: 246 - DNA, 54 bp, Homo sapiens
  source 1..54: mol_type = genomic DNA
cctcatcctcgcagtagctgggtctctcccagggacgcccctagtcagccttgg

SEQ ID NO: 247 - DNA, 132 bp, Homo sapiens
  source 1..132: mol_type = genomic DNA
gtaattatggaattttgcttgggaaattaatttgaaaaagttaattaattggtggttccggaggtggcgggctccacgcccggccagtcttgctgacgtcagtgctgacccactggagacgtgcagcttccg

SEQ ID NO: 248 - DNA, 36 bp, Homo sapiens
  source 1..36: mol_type = genomic DNA
ggggtctgtgacagcttgcccccaaccacggagagg

SEQ ID NO: 249 - DNA, 25 bp, Homo sapiens
  source 1..25: mol_type = genomic DNA
ttgtccagggtggtctcaaaactcc

SEQ ID NO: 250 - DNA, 91 bp, Homo sapiens
  source 1..91: mol_type = genomic DNA
ctgagcagcgaggcccaccaggcatctctgttgtgggcagcagggccaggtcctggtctgtggaccctcggcagttggcaggctccctctg

SEQ ID NO: 251 - DNA, 28 bp, Homo sapiens
  source 1..28: mol_type = genomic DNA
gctcccaggatgccggcggcagtagcag

SEQ ID NO: 252 - DNA, 45 bp, Homo sapiens
  source 1..45: mol_type = genomic DNA
caggagcagcagacaagagtgcagtggtggctgccgccgcaccag

SEQ ID NO: 253 - DNA, 66 bp, Homo sapiens
  source 1..66: mol_type = genomic DNA
cccgagcgtcggaacaagagcggtatcatcagtgagcccctcaacaagagcctgcgccgctcccgc

SEQ ID NO: 254 - DNA, 405 bp, Homo sapiens
  source 1..405: mol_type = genomic DNA
ccatggcggtggacaaaagcaaccctacctcaaagcacaaaagtggtgctgtggccagcctgctgagcaaggcagagcgggccacggagctggcagccgagggacagctgacgctgcagcagtttgcgcagtccacagagatgctgaagcgcgtggtgcaggagcatctcccgctgatgagcgaggcgggtgctggcctgcctgacatggaggctgtggcaggtgccgaagccctcaatggccagtccgacttcccctacctgggcgctttccccatcaacccaggcctcttcattatgaccccggcaggtgtgttcctggccgagagcgcgctgcacatggcgggcctggctgagtaccccatgcagggagagctggcctctgccatcagctccggcaagaaga

SEQ ID NO: 255 - DNA, 47 bp, Homo sapiens
  source 1..47: mol_type = genomic DNA
ttcagaaaatgtgaggaactcaaaaagaagccttccgctgctctgga

SEQ ID NO: 256 - DNA, 42 bp, Homo sapiens
  source 1..42: mol_type = genomic DNA
gtgatgcttccgacgggagccgccttccggtggtttcagtga

SEQ ID NO: 257 - DNA, 161 bp, Homo sapiens
  source 1..161: mol_type = genomic DNA
aatgtcactgctcgtgtggtctccagcaagggattcgggcgaagacaaacggatgcacccgtctttagaaccaaaaatattctctcacagatttcattcctgtttttatatatatattttttgttgtcgttttaacatctccacgtccctagcataaaaag

SEQ ID NO: 258 - DNA, 222 bp, Homo sapiens
  source 1..222: mol_type = genomic DNA
atctataaagtaccgagacttcctgggcaaagaatggacaatcagtttccttcctgtgtcgatgtcgatgttgtctgtgcaggagatgcagtttttgtgtagagaatgtaaattttctgtaaccttttgaaatctagttactaataagcactactgtaatttagcacagtttaactccaccctcatttaaacttcctttgattctttccgaccatgaaatag

SEQ ID NO: 259 - DNA, 84 bp, Homo sapiens
  source 1..84: mol_type = genomic DNA
cctggagaatccactcacgttcataaagagaatgttgatggcgccgtgtagaagccgctctgtatccatccacgcgtgcagagc

SEQ ID NO: 260 - DNA, 118 bp, Homo sapiens
  source 1..118: mol_type = genomic DNA
ggcttagatggctccgagcccgtttgagcgtggtctcggactgctaactggaccaacggcaactgtctgatgagtgccagccccaaaccgcgcgctgctcgggaccttagagcctctg

SEQ ID NO: 261 - DNA, 79 bp, Homo sapiens
  source 1..79: mol_type = genomic DNA
tgttccgctgtttagctcttgttttttgtgtggacactcctaggatagaaagtttggtatgttgctatacctttgcttc

SEQ ID NO: 262 - DNA, 27 bp, Homo sapiens
  source 1..27: mol_type = genomic DNA
gcacccgtgtggttgctaaggatgggc

SEQ ID NO: 263 - DNA, 173 bp, Homo sapiens
  source 1..173: mol_type = genomic DNA
tatacaaggctgcagtcggatacactggtattgtggacgtggcctggagctggacgagacatttagtgtactttttgggcaattggagtcgtttgttattggtcctttttcatttttaatatcttaatgagatgatttaaggaagttactgaatctctgctattaggcctatc

SEQ ID NO: 264 - DNA, 53 bp, Homo sapiens
  source 1..53: mol_type = genomic DNA
gatctcaagtttcaacaccacgttttggcaaaacgttcgatgccccaccagcc

SEQ ID NO: 265 - DNA, 90 bp, Homo sapiens
  source 1..90: mol_type = genomic DNA
gtaagtgttggctataaagacactgtttaaacacttaagcacttttgactcttaaaatgactattggcatcatcctacgtagctttcttc

SEQ ID NO: 266 - DNA, 57 bp, Homo sapiens
  source 1..57: mol_type = genomic DNA
ctgcctcagatgatgcctatccagaaatagaaaaattctttcccttcaatcctctag

SEQ ID NO: 267 - DNA, 208 bp, Homo sapiens
  source 1..208: mol_type = genomic DNA
atgagactgtctgaatctgggttgctttggacaagtgtacttgttgatggaattatttgcaaggtatcatcttaggtcaggaggggaataggaacaaagatgtagaagacattgttcctgtctgtaaaagcttatcacctagaggaggtaagatgtattcatgaacattgaataagtcccattgtggacagtctttctcacaaggctt

SEQ ID NO: 268 - DNA, 70 bp, Homo sapiens
  source 1..70: mol_type = genomic DNA
ctgaagagcaccagattgcgcacctccccttgagtggagtgcctctcatgatccttgacgaggagagaga

SEQ ID NO: 269 - DNA, 74 bp, Homo sapiens
  source 1..74: mol_type = genomic DNA
ctgttgcagtctccttcaagcattctgtcgaccctggatgttgaattgccacctgtttgctgtgacatagatat

SEQ ID NO: 270 - DNA, 31 bp, Homo sapiens
  source 1..31: mol_type = genomic DNA
tcttagtgcttcagagtttgtgtgtatttgt

SEQ ID NO: 271 - DNA, 82 bp, Homo sapiens
  source 1..82: mol_type = genomic DNA
ccgccgtcgcgggtacattcctcgctcccggccgaggagcgctcgggctgtctgcggaccctgccgcgtgcaggggtcgcgg

SEQ ID NO: 272 - DNA, 39 bp, Homo sapiens
  source 1..39: mol_type = genomic DNA
ggcagttggtgggaagtccagcttgggtccctgagagct

SEQ ID NO: 273 - DNA, 70 bp, Homo sapiens
  source 1..70: mol_type = genomic DNA
ccctgttgggggtcctgctgagtgtgcctgggcctccagtcttgtccctggaggcctctgaggaagtgga

SEQ ID NO: 274 - DNA, 136 bp, Homo sapiens
  source 1..136: mol_type = genomic DNA
tacaaggagggcagtcgcctggcacctgctggccgtgtacggggctggaggggccgcctagagattgccagcttcctacctgaggatgctggccgctacctctgcctggcacgaggctccatgatcgtcctgcaga

SEQ ID NO: 275 - DNA, 75 bp, Homo sapiens
  source 1..75: mol_type = genomic DNA
ttgacctccagcaacgatgatgaggaccccaagtcccatagggacccctcgaataggcacagttacccccagcaa

SEQ ID NO: 276 - DNA, 67 bp, Homo sapiens
  source 1..67: mol_type = genomic DNA
gctgctcatctgatcactgagaagaggaggcctgtgtgggaacacacggtcattctaggggccttcc

SEQ ID NO: 277 - DNA, 112 bp, Homo sapiens
  source 1..112: mol_type = genomic DNA
ctgcatgcagtacctgcggggaacaccgtcaagttccgctgtccagctgcaggcaaccccacgcccaccatccgctggcttaaggatggacaggcctttcatggggagaacc

SEQ ID NO: 278 - DNA, 27 bp, Homo sapiens
  source 1..27: mol_type = genomic DNA
ctgcgccatcagcactggagtctcgtg

SEQ ID NO: 279 - DNA, 60 bp, Homo sapiens
  source 1..60: mol_type = genomic DNA
acatacacctgcctggtagagaacgctgtgggcagcatccgctataactacctgctagat

SEQ ID NO: 280 - DNA, 132 bp, Homo sapiens
  source 1..132: mol_type = genomic DNA
cggccaacaccacagccgtggtgggcagcgacgtggagctgctgtgcaaggtgtacagcgatgcccagccccacatccagtggctgaagcacatcgtcatcaacggcagcagcttcggagccgacggtttcc

SEQ ID NO: 281 - DNA, 90 bp, Homo sapiens
  source 1..90: mol_type = genomic DNA
aggtcctgtacctgcggaacgtgtcagccgaggacgcaggcgagtacacctgcctcgcaggcaattccatcggcctctcctaccagtctg

SEQ ID NO: 282 - DNA, 30 bp, Homo sapiens
  source 1..30: mol_type = genomic DNA
aggagatgctgcgagatgcccctctgggcc

SEQ ID NO: 283 - DNA, 104 bp, Homo sapiens
  source 1..104: mol_type = genomic DNA
gcccgaggccaggtatacggacatcatcctgtacgcgtcgggctccctggccttggctgtgctcctgctgctggccgggctgtatcgagggcaggcgctccacg

SEQ ID NO: 284 - DNA, 56 bp, Homo sapiens
  source 1..56: mol_type = genomic DNA
ggcgcatcccccacctcacatgtgacagcctgactccagcaggcagaaccaagtct

SEQ ID NO: 285 - DNA, 115 bp, Homo sapiens
  source 1..115: mol_type = genomic DNA
ttccggcaagtcaagctcatccctggtacgaggcgtgcgtctctcctccagcggccccgccttgctcgccggcctcgtgagtctagatctacctctcgacccactatgggagttc

SEQ ID NO: 286 - DNA, 48 bp, Homo sapiens
  source 1..48: mol_type = genomic DNA
ccctaggcgagggctgctttggccaggtagtacgtgcagaggcctttg

SEQ ID NO: 287 - DNA, 39 bp, Homo sapiens
  source 1..39: mol_type = genomic DNA
aggtgatgaagctgatcggccgacacaagaacatcatca

SEQ ID NO: 288 - DNA, 114 bp, Homo sapiens
  source 1..114: mol_type = genomic DNA
ccctgtacgtgatcgtggagtgcgccgccaagggaaacctgcgggagttcctgcgggcccggcgccccccaggccccgacctcagccccgacggtcctcggagcagtgaggggc

SEQ ID NO: 289 - DNA, 32 bp, Homo sapiens
  source 1..32: mol_type = genomic DNA
cggcgtccaccacattgactactataagaaaa

SEQ ID NO: 290 - DNA, 31 bp, Homo sapiens
  source 1..31: mol_type = genomic DNA
ggccttgtttgaccgggtgtacacacaccag

SEQ ID NO: 291 - DNA, 89 bp, Homo sapiens
  source 1..89: mol_type = genomic DNA
tcgggggctccccgtatcctggcatcccggtggaggagctgttctcgctgctgcgggagggacatcggatggaccgacccccacactgc

SEQ ID NO: 292 - DNA, 26 bp, Homo sapiens
  source 1..26: mol_type = genomic DNA
ctgaggctccctgtgaccctccgccc

SEQ ID NO: 293 - DNA, 38 bp, Homo sapiens
  source 1..38: mol_type = genomic DNA
acgggctgatgcgtgagtgctggcacgcagcgccctcc

SEQ ID NO: 294 - DNA, 110 bp, Homo sapiens
  source 1..110: mol_type = genomic DNA
ctcgacctccgcctgaccttcggaccctattccccctctggtggggacgccagcagcacctgctcctccagcgattctgtcttcagccacgaccccctgccattgggatc

SEQ ID NO: 295 - DNA, 215 bp, Homo sapiens
  source 1..215: mol_type = genomic DNA
cctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccttggcgcttagtccccatcccgggtttggctgagcctggctggagagctgctatgctaaacctcctgcc

SEQ ID NO: 296 - DNA, 229 bp, Homo sapiens
  source 1..229: mol_type = genomic DNA
agaagggcgcctgcttgttctttctttttgtctgctttcccccgtttgcgcctggaagctgcgccgcgagttcctgcaaggcggtctgccgcggccgggcccggccttctcccctcgcagcgaccccgcctcgcggccgcgcgggccccgaggtagcccgaggcgccggaggagccagccccagcgagcgccgggagaggcggcagcgcagccggacgcacagcgcagc

SEQ ID NO: 297 - DNA, 126 bp, Homo sapiens
  source 1..126: mol_type = genomic DNA
atgcaggcgcgctactccgtgtccagccccaactccctgggagtggtgccctacctcggcggcgagcagagctactaccgcgcggcggccgcggcggccgggggcggctacaccgccatgccggcc

SEQ ID NO: 298 - DNA, 177 bp, Homo sapiens
  source 1..177: mol_type = genomic DNA
atgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaac

SEQ ID NO: 299 - DNA, 32 bp, Homo sapiens
  source 1..32: mol_type = genomic DNA
aggacaggctgcacctcaaggagccgcccccg

SEQ ID NO: 300 - DNA, 26 bp, Homo sapiens
  source 1..26: mol_type = genomic DNA
gccgacggcaacgcgcccggtccgca

SEQ ID NO: 301 - DNA, 29 bp, Homo sapiens
  source 1..29: mol_type = genomic DNA
gcgcatccaggacatcaagaccgagaacg

SEQ ID NO: 302 - DNA, 99 bp, Homo sapiens
  source 1..99: mol_type = genomic DNA
cgccgcggtgcccaagatcgagagccccgacagcagcagcagcagcctgtccagcgggagcagccccccgggcagcctgccgtcggcgcggccgctcag

SEQ ID NO: 303 - DNA, 114 bp, Homo sapiens
  source 1..114: mol_type = genomic DNA
ccgccgccgcaccatagccagggcttcagcgtggacaacatcatgacgtcgctgcgggggtcgccgcagagcgcggccgcggagctcagctccggccttctggcctcggcggcc

SEQ ID NO: 304 - DNA, 177 bp, Homo sapiens
  source 1..177: mol_type = genomic DNA
gtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacct

SEQ ID NO: 305 - DNA, 110 bp, Homo sapiens
  source 1..110: mol_type = genomic DNA
atgagcctgtacgcggccggcgagcgcgggggccacttgcagggcgcgcccgggggcgcgggcggctcggccgtggacgaccccctgcccgactactctctgcctccggt

SEQ ID NO: 306 - DNA, 39 bp, Homo sapiens
  source 1..39: mol_type = genomic DNA
ggccggccaccaccctgcggcccaccaaggccgcctcac

SEQ ID NO: 307 - DNA, 27 bp, Homo sapiens
  source 1..27: mol_type = genomic DNA
acctgggccacttggcgagcgcggcgg

SEQ ID NO: 308 - DNA, 143 bp, Homo sapiens
  source 1..143: mol_type = genomic DNA
cagaacttccactcggtgcgggagatgttcgagtcacagaggatcggcttgaacaactctccagtgaacgggaatagtagctgtcaaatggccttcccttccagccagtctctgtaccgcacgtccggagctttcgtctacga

SEQ ID NO: 309 - DNA, 449 bp, Homo sapiens
  source 1..449: mol_type = genomic DNA
tgagaatattcaccacaccagcgaacagaatatccctccaaaaattcagctcaccagcaccagcacgaagaaaactctattttcttaaccgattaattcagagccacctccactttgccttgtctaaataaacaaacccgtaaactgttttatacagagacagcaaaatcttggtttattaaaggacagtgttactccagataacacgtaagtttcttcttgcttttcagagacctgctttcccctcctcccgtctcccctctcttgccttcttccttgcctctcacctgtaagatattattttatcctatgttgaagggagggggaaagtccccgtttatgaaagtcgctttctttttattcatggacttgttttaaaatgtaaattgcaacatagtaatttatttttaatttgtagttggatgtcgtggaccaaacgccagaaagtg

SEQ ID NO: 310 - DNA, 871 bp, Homo sapiens
  source 1..871: mol_type = genomic DNA
ggagaaaccctctgactagtccatgtcaaattttactaaaagtctttttgtttagatttattttcctgcagcatcttctgcaaaatgtactatatagtcagcttgctttgaggctagtaaaaagatatttttctaaacagattggagttggcatataaacaaatacgttttctcactaatgacagtccatgattcggaaattttaagcccatgaatcagccgcggtcttaccacggtgatgcctgtgtgccgagagatgggactgtgcggccagatatgcacagataaatatttggcttgtgtattccatataaaattgcagtgcatattatacatccctgtgagccagatgctgaatagatattttcctattatttcagtcctttataaaaggaaaaataaaccagtttttaaatgtatgtatataattctcccccatttacaatccttcatgtattacatagaaggattgcttttttaaaaatatactgcgggttggaaagggatatttaatctttgagaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagagg

SEQ ID NO: 311 - DNA, 63 bp, Homo sapiens
  source 1..63: mol_type = genomic DNA
agaagctctttaacaggctcgaaaggtccatgctcctttctcctgcccattctatagcataag

SEQ ID NO: 312 - DNA, 88 bp, Homo sapiens
  source 1..88: mol_type = genomic DNA
caaagatctcttcacattctccgggactgcggtaccaaatatcagcacagcacttcttgaaaaaggatgtagattttaatctgaactt

SEQ ID NO: 313 - DNA, 118 bp, Homo sapiens
  source 1..118: mol_type = genomic DNA
acgaagtggaggagtattacatttcagctggaaacacatccctagaatgccaaaacatttattccaaagtctggtttcctggtgcaatcggaggcatggcaatgcctctgttcagaga

SEQ ID NO: 314 - DNA, 215 bp, Homo sapiens
  source 1..215: mol_type = genomic DNA
aagcatagggtactttccagcctccaagggtaggggcaaaggggctggggtttctcctccccagtacagctttctctggctgtgccacactgctccctgtgagcagacagcaagtctcccctcactccccactgccattcatccagcgctgtgcagtagcccagctgcgtgtctgccgggaggggctgccaagtgccctgcctactggctgcttc

SEQ ID NO: 315 - DNA, 147 bp, Homo sapiens
  source 1..147: mol_type = genomic DNA
gcagcacattagagaaagccggcccctggatccgtctttcgcgtttattttaagcccagtcttccctgggccacctttagcagatcctcgtgcgcccccgccccctggccgtgaaactcagcctctatccagcagcgacgacaagta

SEQ ID NO: 316 - DNA, 256 bp, Homo sapiens
  source 1..256: mol_type = genomic DNA
ccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgct

SEQ ID NO: 317 - DNA, 70 bp, Homo sapiens
  source 1..70: mol_type = genomic DNA
gtggcccgccggtttctgagccttctgccctgcggggacacggtctgcaccctgcccgcggccacggacc

SEQ ID NO: 318 - DNA, 48 bp, Homo sapiens
  source 1..48: mol_type = genomic DNA
tctgggatggccctactgcatcagatccaagggaacgagctggagccc

SEQ ID NO: 319 - DNA, 114 bp, Homo sapiens
  source 1..114: mol_type = genomic DNA
cccctgggcgaggtgtacctggacagcagcaagcccgccgtgtacaactaccccgagggcgccgcctacgagttcaacgccgcggccgccgccaacgcgcaggtctacggtcag

SEQ ID NO: 320 - DNA, 155 bp, Homo sapiens
  source 1..155: mol_type = genomic DNA
tgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagc

SEQ ID NO: 321 - DNA, 209 bp, Homo sapiens
  source 1..209: mol_type = genomic DNA
gtgctgtcatgtggactgtcctcccgagtgtcccactggatgttcagagaatttatgtgaaggtcacgtcatttagcattgagatgctgtggttaccttcttccatttcttccataatatgcagccacatctatgtgtgaagaaatgtaatagataaaatttctctggacgcataataatgtgagaaagattgtcacatgtcccagcaa

SEQ ID NO: 322 - DNA, 62 bp, Homo sapiens
  source 1..62: mol_type = genomic DNA
caaattcagataatcgacgccagggtggcagagaaagattggccagtaccaatgacaaggga

SEQ ID NO: 323 - DNA, 77 bp, Homo sapiens
  source 1..77: mol_type = genomic DNA
aatctgccaaggagactcgctactgtgcagtgtgcaatgactatgcttcaggctaccattatggagtctggtcctgt

SEQ ID NO: 324 - DNA, 40 bp, Homo sapiens
  source 1..40: mol_type = genomic DNA
caggcctgccggctccgtaaatgctacgaagtgggaatga

SEQ ID NO: 325 - DNA, 185 bp, Homo sapiens
  source 1..185: mol_type = genomic DNA
aacctttggccaagcccgctcatgatcaaacgctctaagaagaacagcctggccttgtccctgacggccgaccagatggtcagtgccttgttggatgctgagcccccgatactctattccgagtatgatcctaccagacccttcagtgaagcttcgatgatgggcttactgaccaacctggcaga

SEQ ID NO: 326 - DNA, 96 bp, Homo sapiens
  source 1..96: mol_type = genomic DNA
tggatatatgtgtgatcctgggtgtgccaaatgctgtggcttcctgaagcttagatttccagcttgtcaccttcaaggttaccttgtgaataggac

SEQ ID NO: 327 - DNA, 111 bp, Homo sapiens
  source 1..111: mol_type = genomic DNA
tgactatggattttgcctgttgctttgtttccaccaactctccctgaagatgaggcgcacagacagacaactcacaggcaagaacagcctggtccatcttgaaagattctc

SEQ ID NO: 328 - DNA, 29 bp, Homo sapiens
  source 1..29: mol_type = genomic DNA
gctttgtggatttgaccctccatgatcag

SEQ ID NO: 329 - DNA, 94 bp, Homo sapiens
  source 1..94: mol_type = genomic DNA
gtccaccttctagaatgtgcctggctagagatcctgatgattggtctcgtctggcgctccatggagcacccagggaagctactgtttgctccta

SEQ ID NO: 330 - DNA, 76 bp, Homo sapiens
  source 1..76: mol_type = genomic DNA
tggagatcttcgacatgctgctggctacatcatctcggttccgcatgatgaatctgcagggagaggagtttgtgtg

SEQ ID NO: 331 - DNA, 41 bp, Homo sapiens
  source 1..41: mol_type = genomic DNA
gagaaggaccatatccaccgagtcctggacaagatcacaga

SEQ ID NO: 332 - DNA, 157 bp, Homo sapiens
  source 1..157: mol_type = genomic DNA
catgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttg

SEQ ID NO: 333 - DNA, 3214 bp, Homo sapiens
  source 1..3214: mol_type = genomic DNA
cctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaa

SEQ ID NO: 334 - DNA, 33 bp, Homo sapiens
  source 1..33: mol_type = genomic DNA
agaagagaatcctgaacttgcatcctaaaatat

SEQ ID NO: 335 - DNA, 1065 bp, Homo sapiens
  source 1..1065: mol_type = genomic DNA
ggcaacttgttgactacccactggtcattctcctctggtcttattacatacatggatgccagtttagattgtgtttatataggaaaaattaaatgtgtgagcctccttaaggaacatcatcaatacagatatatcagatagttctgtccagcaaaaaacgtgcttatttgctacaagtaaatttttatttatttttctcacttccctcactccttcaaatttccaggtaaatagctgcccaggagttgcttcatctctgtcccaaaatacctagacaattgcgggataaggagaatggcagggagggagtagtggctaaaatcacacccttcaaaagaaagtgtgtaggacacacaattgtgagaagtctgaatgccatgcacatagggtatgactcactttgaaaattgtttataatcaaggaaatgaaaatgagttaatttcgtgcatgcatcatttaaagccaaatgagaagaaacttctaatttattttgttacttttcggctaacactggcagtatgtaacagatttattttgcagaaacatctagattgtccgtgatcttgatcctgcccttatgtgtcttgtctttgaaacccagtgtttcctggatatatggttcaggagacaagtttccagaatcaagttaggacccaggtcttctttttttccaaaccaaacattcttgctaatcctaaactacctgaggcagcctgtggtggcctcagctctaaaaccattgtttaaaggcttctacccatcaatggcccttcagcagagtggtacggttaacggggtagggtctggagtcaggggagacctgggttcaaatcctacatctttacacctctaatccccagtgtccttgtctataaattgggaatatagccatgtcatgggattcttgtgagggttaaatgaggtaaaacacatacaatgcttagcatgtatacaattaagcactaaataattgaaacacattaagtactaaatgaatgtcagcagcttatcactattatctgtataatgataccaagggtgtgccgactcatacccttaggggt

SEQ ID NO: 336 - DNA, 30 bp, Homo sapiens
  source 1..30: mol_type = genomic DNA
aggaaggcctacctcaaatagcaacagaga

SEQ ID NO: 337 - DNA, 33 bp, Homo sapiens
  source 1..33: mol_type = genomic DNA
tctgtgctgaggctctttgaatgctttgaataa

SEQ ID NO: 338 - DNA, 1603 bp, Homo sapiens
  source 1..1603: mol_type = genomic DNA
cctctttcagtgtttcggccagtcatttgccacttctcattccatcttagttctctgtaaagaaggtgccagagacctaaggtgcccaaggcaattttgcattttacaattctaagctttagaatgaagtcatcaatttgctacatccggactacagtgcaattattcctttgccttgctggaaattggagtgaaatctttctagctgtcaatttcaactcagttgcagtagtgttttgaagaattaatggcgataaggttagaaaattttaagtcaaacgtagggaaaaagtaccagctagaccatcataagcatttgctttgaaagcatgcttctaaagtgtgtttaacctcaaataacagtcacaaatatggttattatgaatgtatgcacagatttttatgtttctaattttaagaagttctagggagctccctgtaacgatttagggaatctctagattctgatatactgcaagtcttttaatggtaggaatcacattgaattaattttgtaggcccagggcctaaatttagtaggtgttcagtacctattggcatcaattcatatgtaggtttaaaatactgtatgaagatacagaatcaccaccatcaaatcaaattgaaatatgtaacaggctagtataatattaacatctgactttaaacaacaacaaagaaaccaaatgagtaactcctcccttcaaactaatagtcagtttcttccaactcagtctctttctcctctcaggaagaatgcgtatctaaaaatttcccattgcagactgctggaaacaacattctaaactatttatgcttctgcaataacctttccaatttgctggaccagtgcaagattaaacacgagatatctcaagtctcaatgtaaaggaacaccacgacagcctggactgtgggtgaagttcattcttccccagcagactctgcctttcattctcggggttgggtgtgccccaaacagaggtaccgacggtaacgaagcccaagaatgttcaaccacaacctgtctgtgaaggtgttggatgacgtttgccattcaggtgaagattatttatgttccagtcccacctgagtagcaaagtgaacactgtgctgaatgctcagaaagatgttaatgaaccgtgctggacagagcagagctgaaaggcgccttgcgagtgtcgtagtgagaatgtggctgtcccagctgcaaagccctgttaggaggcatgaggaagcacttgctgccctaagaaacgatgccttcgacattttcaaaagatctatgtggctgtctgaaacaatgcggagagcagatagacgcaatatttgggaaccaaagagtgactgctgttggcgttgcatcataacataagcgctttcccccttctcgtcactatcatttgtatcaaccaaagaactgatctctggtatcctcgaaggaatgctgtggggatattcttcatctctgttcatggtacatcagcaatttgtggggaaaagatggactatataacacaatgatctgcctaaaagaaactgtctctacttatagggggctgagcaaaccttagagcatctgcggatgctcgtcattatcttc

SEQ ID NO: 339 - DNA, 94 bp, Homo sapiens
  source 1..94: mol_type = genomic DNA
gaaattcaaatttgaacggctgcagaggccgagtccgtcactggaagccgagaggagaggacagctggttgtgggagagttcccccgcctcaga

SEQ ID NO: 340 - DNA, 78 bp, Homo sapiens
  source 1..78: mol_type = genomic DNA
acacactgagctgagactcacttttctcttcctgaatttgaaccaccgtttccatcgtctcgtagtccgacgcctggg

SEQ ID NO: 341 - DNA, 52 bp, Homo sapiens
  source 1..52: mol_type = genomic DNA
ggaggaaggctttgagtctgtcctaaaaggctgttgcgagaggtctttcagc

SEQ ID NO: 342 - DNA, 176 bp, Homo sapiens
  source 1..176: mol_type = genomic DNA
tcctggcgcagcaagagtgaggcgcaggcctgcggaacgggtcctgctggaagcagctggaatgccctgcagggcggggtccggggccggtgactcagtgcggctgccgccgggaaaggcagtaggatgtgtgatttgcggagttcacgcagcccgcaggggagatgctaatgaaa

SEQ ID NO: 343 - DNA, 45 bp, Homo sapiens
  source 1..45: mol_type = genomic DNA
aactgctggagcgaacccgtgccaggcgagagaatcttcagagaa

SEQ ID NO: 344 - DNA, 52 bp, Homo sapiens
  source 1..52: mol_type = genomic DNA
cagcagctccaaggtctatgactcatgctaagcgagctagacagccactttc

SEQ ID NO: 345 - DNA, 137 bp, Homo sapiens
  source 1..137: mol_type = genomic DNA
agaaatcttgtacaaaaccatcgccatcaaaaaaacgctgttctgacaacactgaagtagaagtttctaacttggaaaataaacaaccagttgagtcgacatctgcaaaatcttgttctccaagtcctgtgtctcct

SEQ ID NO: 346 - DNA, 167 bp, Homo sapiens
  source 1..167: mol_type = genomic DNA
gccacaagcagcagataccatcagtgattctgttgctgtcccggcatcactgctgggcatgaggagagggctgaactcaagattggaagcaactgcagcctcctcagttaaaacacgtatgcaaaaacttgcagagcaacggcgccgttgggataatgatgatatga

SEQ ID NO: 347 - DNA, 79 bp, Homo sapiens
  source 1..79: mol_type = genomic DNA
gctttcaaatgcctcggcaactccagttggcagaaggggccgtctggccaatcttgctgcaactatttgctcctgggaa

SEQ ID NO: 348 - DNA, 152 bp, Homo sapiens
  source 1..152: mol_type = genomic DNA
gcctggtaccgcttgtttatccaaattttcctctgcaagtggagcatctgctaggatcaatagcagcagtgttaagcaggaagctacattctgttcccaaagggatggcgatgcctctttgaataaagccctatcctcaagtgctgatgatg

SEQ ID NO: 349 - DNA, 152 bp, Homo sapiens
  source 1..152: mol_type = genomic DNA
tccagtgaaatctactacatctatcactgatgctaaaagttgtgagggacaaaatcctgagctacttccaaaaactcctattagtcctctgaaaacgggggtatcgaaaccaattgtgaagtcaactttatcccagacagttccatccaagg

SEQ ID NO: 350 - DNA, 115 bp, Homo sapiens
  source 1..115: mol_type = genomic DNA
agcctttcctggaacgctttggagagcgttgtcaagaacatagcaaagaaagtccagctcgtagcacaccccacagaacccccattattactccaaatacaaaggccatccaaga

SEQ ID NO: 351 - DNA, 72 bp, Homo sapiens
  source 1..72: mol_type = genomic DNA
agaactagcatgtcttcgtggccgatttgacaagggcaatatatggagtgcagaaaaaggcggaaactcaaa

SEQ ID NO: 352 - DNA, 76 bp, Homo sapiens
  source 1..76: mol_type = genomic DNA
gtttcaaaaactcagtcacttccagtaacagaaaaggtgaccgaaaaccagataccagccaaaaattctagtacag

SEQ ID NO: 353 - DNA, 25 bp, Homo sapiens
  source 1..25: mol_type = genomic DNA
catcagacccaaaggttgagcagaa

SEQ ID NO: 354 - DNA, 151 bp, Homo sapiens
  source 1..151: mol_type = genomic DNA
tcttcagtgatgtcctagaggaaggtgaactagatatggagaagagccaagaggagatggatcaagcattagcagaaagcagcgaagaacaggaagatgcactgaatatctcctcaatgtctttacttgcaccattggcacaaacagttgg

SEQ ID NO: 355 - DNA, 44 bp, Homo sapiens
  source 1..44: mol_type = genomic DNA
agtttagtgtccacacctagactggaattgaaagacaccagcag

SEQ ID NO: 356 - DNA, 59 bp, Homo sapiens
  source 1..59: mol_type = genomic DNA
ttcaaagaaacagaacgtccatcaataaagcaggtgattgttcggaaggaagatgttac

SEQ ID NO: 357 - DNA, 135 bp, Homo sapiens
  source 1..135: mol_type = genomic DNA
gaactcaataacgaaataaatatgcaacagacagtgatctatcaagctagccaggctcttaactgctgtgttgatgaagaacatggaaaagggtccctagaagaagctgaagcagaaagacttcttctaattgca

SEQ ID NO: 358 - DNA, 76 bp, Homo sapiens
  source 1..76: mol_type = genomic DNA
tgccatccaaaggatcagttactttgtcagaaatccgcttgcctctaaaagcagattttgtctgcagtacggttca

SEQ ID NO: 359 - DNA, 76 bp, Homo sapiens
  source 1..76: mol_type = genomic DNA
atggtagccacaccattagcaagtacttcaaactctcttaacggtgatgctctgacattcactactacatttactc

SEQ ID NO: 360 - DNA, 30 bp, Homo sapiens
  source 1..30: mol_type = genomic DNA
aagaaagatccctcaggccttgataagaag

SEQ ID NO: 361 - DNA, 75 bp, Homo sapiens
  source 1..75: mol_type = genomic DNA
ggccagtccaggaggtcttagtgctgtgcgaaccagcaacttcgcccttgttggatcttacacattatcattgtc

SEQ ID NO: 362 - DNA, 40 bp, Homo sapiens
  source 1..40: mol_type = genomic DNA
taagagagcgagagctactgggctatttgttccaggaaaa

SEQ ID NO: 363 - DNA, 75 bp, Homo sapiens
  source 1..75: mol_type = genomic DNA
gttttggtgcctggcatcgaagatggtgtgttctttctggaaactgtatatcttattggacttatccagatgatg

SEQ ID NO: 364 - DNA, 378 bp, Homo sapiens
  source 1..378: mol_type = genomic DNA
ttggctgttggctcatgtgtgcctatatgtgtttcttttcccattttcaggaccatttggtctcgtgaatgttttcctccactttgactcgtatcataggaattcatggctgccaacaatccagggcagttgtctgcccttatctttcatagatatataaagaaatatttacacatgaaatccaatgtctaggtttccttttatagaaaggggagaagtgggtaagttgtagataaaaggcacttgagtgtgtttctcattgttatagctggttttggtacctggggctcattatactgttgtttgtattttttatttgaagttcaccataataaagagctttataggatagttggcaagagctaccagttgatattt

SEQ ID NO: 365 - DNA, 33 bp, Homo sapiens
  source 1..33: mol_type = genomic DNA
cataggaaggataaatctggctaattgtaccag

SEQ ID NO: 366 - DNA, 96 bp, Homo sapiens
  source 1..96: mol_type = genomic DNA
gtgcaagacgcaacacttttgaattaattactgtccgaccacaaagagaagatgaccgagagactcttgtcagccaatgcagggacacactctgtg

SEQ ID NO: 367 - DNA, 81 bp, Homo sapiens
  source 1..81: mol_type = genomic DNA
tggctgtctgcagatactaaagaagagcgggatctctggatgcaaaaactcaatcaagttcttgttgatattcgcctctgg

SEQ ID NO: 368 - DNA, 27 bp, Homo sapiens
  source 1..27: mol_type = genomic DNA
ttgctacaaacctattggaaagcctta

SEQ ID NO: 369 - DNA, 645 bp, Homo sapiens
  source 1..645: mol_type = genomic DNA
tacgaaagggtttgtgccaatattcactacgtattatgcagtatttatatcttttgtatgtaaaactttaactgatttctgtcattcatcaatgagtagaagtaaatacattatagttgattttgctaaatcttaatttaaaagcctcattttcctagaaatctaattattcagttattcatgacaatatttttttaaaagtaagaaattctgagttgtcttcttggagctgtaggtcttgaagcagcaacgtctttcaggggttggagacagaaacccattctccaatctcagtagttttttcgaaaggctgtgatcatttattgatcgtgatatgacttgttactagggtactgaaaaaaatgtctaaggcctttacagaaacatttttagtaatgaggatgagaactttttcaaatagcaaatatatattggcttaaagcatgaggctgtcttcagaaaagtgatgtggacataggaggcaatgtgtgagacttgggggttcaatattttatatagaagagttaataagcacatggtttacatttactcagctactatatatgcagtgtggtgcacattttcacagaattctggcttcattaagatcattatttttgctgcgtagcttacagacttagcata

SEQ ID NO: 370 - DNA, 120 bp, Homo sapiens
  source 1..120: mol_type = genomic DNA
ccgagaggaagtttggcgtggtggtggttggtgttggccgagccggctccgtgcggatgagggacttgcggaatccacacccttcctcagcgttcctgaacctgattggcttcgtgtcga

SEQ ID NO: 371 - DNA, 100 bp, Homo sapiens
  source 1..100: mol_type = genomic DNA
agctcgggagcattgatggagtccagcagatttctttggaggatgctctttccagccaagaggtggaggtcgcctatatctgcagtgagagctccagcca

SEQ ID NO: 372 - DNA, 74 bp, Homo sapiens
  source 1..74: mol_type = genomic DNA
ttccttaatgctggcaagcacgtccttgtggaataccccatgacactgtcattggcggccgctcaggaactgtg

SEQ ID NO: 373 - DNA, 104 bp, Homo sapiens
  source 1..104: mol_type = genomic DNA
gaaaagtcttgcacgaggagcatgttgaactcttgatggaggaattcgctttcctgaaaaaagaagtggtggggaaagacctgctgaaagggtcgctcctcttc

SEQ ID NO: 374 - DNA, 124 bp, Homo sapiens
  source 1..124: mol_type = genomic DNA
agaagagcggtttggcttccctgcattcagcggcatctctcgcctgacctggctggtctccctctttggggagctttctcttgtgtctgccactttggaagagcgaaaggaagatcagtatatg

SEQ ID NO: 375 - DNA, 124 bp, Homo sapiens
  source 1..124: mol_type = genomic DNA
cctggtctaaaacgaaacagatatttaagcttccatttcaagtctgggtccttggagaatgtgccaaatgtaggagtgaataagaacatatttctgaaagatcaaaatatatttgtccagaaac

SEQ ID NO: 376 - DNA, 42 bp, Homo sapiens
  source 1..42: mol_type = genomic DNA
ggttcttctcaagagttgaccattatctctattcttaaaatt

SEQ ID NO: 377 - DNA, 28 bp, Homo sapiens
  source 1..28: mol_type = genomic DNA
ggctgcgctctgcccggcgagtcgggct

SEQ ID NO: 378 - DNA, 31 bp, Homo sapiens
  source 1..31: mol_type = genomic DNA
atgatcctttgcctggactttctaagtgccc

SEQ ID NO: 379 - DNA, 121 bp, Homo sapiens
  source 1..121: mol_type = genomic DNA
gttgcacttggcctagcattccaacctcacctgcctcagcttgttcaacctgaaaacctaccaagtgaaagcaagagccacgtgaagacgccttagttatatgcacccacccagacacttg

SEQ ID NO: 380 - DNA, 186 bp, Homo sapiens
  source 1..186: mol_type = genomic DNA
aactacaggccttttgagagagtgccctcctaatgaattgagtacctatttctccatacacagtgtctatcatgacctacaaacccttttcccatgaggtgtaacagagagagattacagccttggaactggatgtcagactctcctggtttaagacaataagccatgacatagagcctgaaacca

SEQ ID NO: 381 - DNA, 124 bp, Homo sapiens
  source 1..124: mol_type = genomic DNA
caagctcacgcagttgggcacttttgaagatcattttctcagcctccagaggatgttcaataactgtgaggtggtccttgggaatttggaaattacctatgtgcagaggaattatgatctttcc

SEQ ID NO: 382 - DNA, 120 bp, Homo sapiens
  source 1..120: mol_type = genomic DNA
aggtggctggttatgtcctcattgccctcaacacagtggagcgaattcctttggaaaacctgcagatcatcagaggaaatatgtactacgaaaattcctatgccttagcagtcttatcta

SEQ ID NO: 383 - DNA, 101 bp, Homo sapiens
  source 1..101: mol_type = genomic DNA
ggcgccgtgcggttcagcaacaaccctgccctgtgcaacgtggagagcatccagtggcgggacatagtcagcagtgactttctcagcaacatgtcgatgga

SEQ ID NO: 384 - DNA, 41 bp, Homo sapiens
  source 1..41: mol_type = genomic DNA
gccaaaagtgtgatccaagctgtcccaatgggagctgctgg

SEQ ID NO: 385 - DNA, 109 bp, Homo sapiens
  source 1..109: mol_type = genomic DNA
aaaatcatctgtgcccagcagtgctccgggcgctgccgtggcaagtcccccagtgactgctgccacaaccagtgtgctgcaggctgcacaggcccccgggagagcgact

SEQ ID NO: 386 - DNA, 113 bp, Homo sapiens
  source 1..113: mol_type = genomic DNA
cgaagccacgtgcaaggacacctgccccccactcatgctctacaaccccaccacgtaccagatggatgtgaaccccgagggcaaatacagctttggtgccacctgcgtgaaga

SEQ ID NO: 387 - DNA, 96 bp, Homo sapiens
  source 1..96: mol_type = genomic DNA
taattatgtggtgacagatcacggctcgtgcgtccgagcctgtggggccgacagctatgagatggaggaagacggcgtccgcaagtgtaagaagtg

SEQ ID NO: 388 - DNA, 25 bp, Homo sapiens
  source 1..25: mol_type = genomic DNA
tggatattctgaaaaccgtaaagga

SEQ ID NO: 389 - DNA, 48 bp, Homo sapiens
  source 1..48: mol_type = genomic DNA
tgttttagagagagaacttttcgacatatttcctgttcccttggaata

SEQ ID NO: 390 - DNA, 110 bp, Homo sapiens
  source 1..110: mol_type = genomic DNA
ttttctcttgcagtcgtcagcctgaacataacatccttgggattacgctccctcaaggagataagtgatggagatgtgataatttcaggaaacaaaaatttgtgctatgc

SEQ ID NO: 391 - DNA, 127 bp, Homo sapiens
  source 1..127: mol_type = genomic DNA
cacaggccaggtctgccatgccttgtgctcccccgagggctgctggggcccggagcccagggactgcgtctcttgccggaatgtcagccgaggcagggaatgcgtggacaagtgcaaccttctggag

SEQ ID NO: 392 - DNA, 123 bp, Homo sapiens
  source 1..123: mol_type = genomic DNA
tgtatccagtgtgcccactacattgacggcccccactgcgtcaagacctgcccggcaggagtcatgggagaaaacaacaccctggtctggaagtacgcagacgccggccatgtgtgccacctg

SEQ ID NO: 393 - DNA, 163 bp, Homo sapiens
  source 1..163: mol_type = genomic DNA
gccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctg

SEQ ID NO: 394 - DNA, 326 bp, Homo sapiens
  source 1..326: mol_type = genomic DNA
gcctcatgccttcacgtgtctgttccccccgcttttcctttctgccacccctgcacgtgggccgccaggttcccaagagtatcctacccatttccttccttccactccctttgccagtgcctctcaccccaactagtagctaaccatcacccccaggactgacctcttcctcctcgctgccagatgattgttcaaagcacagaatttgtcagaaacctgcagggactccatgctgccagccttctccgtaattagcatggccccagtccatgcttctagccttggttccttctgcccctctgtttgaaattctagagccagctgtg

SEQ ID NO: 395 - DNA, 25 bp, Homo sapiens
  source 1..25: mol_type = genomic DNA
gccaggtcttgaaggctgtccaacg

SEQ ID NO: 396 - DNA, 25 bp, Homo sapiens
  source 1..25: mol_type = genomic DNA
aagctacatagtgtctcactttcca

SEQ ID NO: 397 - DNA, 28 bp, Homo sapiens
  source 1..28: mol_type = genomic DNA
gcctaagatcccgtccatcgccactggg

SEQ ID NO: 398 - DNA, 60 bp, Homo sapiens
  source 1..60: mol_type = genomic DNA
gtggccctggggatcggcctcttcatgcgaaggcgccacatcgttcggaagcgcacgctg

SEQ ID NO: 399 - DNA, 112 bp, Homo sapiens
  source 1..112: mol_type = genomic DNA
gcctcttacacccagtggagaagctcccaaccaagctctcttgaggatcttgaaggaaactgaattcaaaaagatcaaagtgctgggctccggtgcgttcggcacggtgtat

SEQ ID NO: 400 - DNA, 59 bp, Homo sapiens
  source 1..59: mol_type = genomic DNA
ttaaaattcccgtcgctatcaaggaattaagagaagcaacatctccgaaagccaacaag

SEQ ID NO: 401 - DNA, 162 bp, Homo sapiens
  source 1..162: mol_type = genomic DNA
ctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactg

SEQ ID NO: 402 - DNA, 139 bp, Homo sapiens
  source 1..139: mol_type = genomic DNA
ggcatgaactacttggaggaccgtcgcttggtgcaccgcgacctggcagccaggaacgtactggtgaaaacaccgcagcatgtcaagatcacagattttgggctggccaaactgctgggtgcggaagagaaagaatacc

SEQ ID NO: 403 - DNA, 62 bp, Homo sapiens
  source 1..62: mol_type = genomic DNA
gtgcctatcaagtggatggcattggaatcaattttacacagaatctatacccaccagagtga

SEQ ID NO: 404 - DNA, 117 bp, Homo sapiens
  source 1..117: mol_type = genomic DNA
agttgatgacctttggatccaagccatatgacggaatccctgccagcgagatctcctccatcctggagaaaggagaacgcctccctcagccacccatatgtaccatcgatgtctaca

SEQ ID NO: 405 - DNA, 72 bp, Homo sapiens
  source 1..72: mol_type = genomic DNA
ctggatgatagacgcagatagtcgcccaaagttccgtgagttgatcatcgaattctccaaaatggcccgaga

SEQ ID NO: 406 - DNA, 241 bp, Homo sapiens
  source 1..241: mol_type = genomic DNA
gcttccattgggaagagtccctctaatgagcatctcatgtcactgtgttctgtcacatgccagcctggcctccctgtgtcccagatcgcattattaaaccctccagcgcattagagcaagcctcagtaaggcgcaggccacatcgtgaactaagcagcatccgtgagtggggcccacccaactccatctccccctccccgtctgaactctcctctggtgctcgtcctcactgtccggctag

SEQ ID NO: 407 - DNA, 109 bp, Homo sapiens
  source 1..109: mol_type = genomic DNA
ggggatgaaagaatgcatttgccaagtcctacagactccaacttctaccgtgccctgatggatgaagaagacatggacgacgtggtggatgccgacgagtacctcatcc

SEQ ID NO: 408 - DNA, 75 bp, Homo sapiens
  source 1..75: mol_type = genomic DNA
gacagcttcttgcagcgatacagctcagaccccacaggcgccttgactgaggacagcatagacgacaccttcctc

SEQ ID NO: 409 - DNA, 34 bp, Homo sapiens
  source 1..34: mol_type = genomic DNA
tcctgctcctcaacctcctcgacccactcagcag

SEQ ID NO: 410 - DNA, 30 bp, Homo sapiens
  source 1..30: mol_type = genomic DNA
cctccagcatctccagagggggaaacagtg

SEQ ID NO: 411 - DNA, 181 bp, Homo sapiens
  source 1..181: mol_type = genomic DNA
gtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaat

SEQ ID NO: 412 - DNA, 1324 bp, Homo sapiens
  source 1..1324: mol_type = genomic DNA
ccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttt

SEQ ID NO: 413 - DNA, 129 bp, Homo sapiens
  source 1..129: mol_type = genomic DNA
ggcaccctgaccgaggaaacagctgccagaggcctccactgctaaagtccacataaggctgaggtcagtcaccctaaacaacctgctccctctaagccaggggatgagcttggagcatcccacaagttc

SEQ ID NO: 414 - DNA, 85 bp, Homo sapiens
  source 1..85: mol_type = genomic DNA
gaaatatttcagtcagaactgggaaacagaaggacctacattctgctgtcacttatgtgtcaagaagcagatgatcgatgaggca

SEQ ID NO: 415 - DNA, 71 bp, Homo sapiens
  source 1..71: mol_type = genomic DNA
gtcagttgtaagtgagtcacattgtagcattaaattctagtatttttgtagtttgaaacagtaacttaata

SEQ ID NO: 416 - DNA, 51 bp, Homo sapiens
  source 1..51: mol_type = genomic DNA
cacagttctgtctggtagaagccgcaaagcccttagcctcttcacggatct

SEQ ID NO: 417 - DNA, 128 bp, Homo sapiens
  source 1..128: mol_type = genomic DNA
gatagcctggccttaataccctacagaaagcctgtccattggctgtttcttcctcagtcagttcctggaagaccttaccccatgaccccagcttcagatgtggtctttggaaacagaggtcgaaggaa

SEQ ID NO: 418 - DNA, 1828 bp, Homo sapiens
  source 1..1828: mol_type = genomic DNA
ctaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaag

SEQ ID NO: 419 - DNA, 32 bp, Homo sapiens
  source 1..32: mol_type = genomic DNA
gaaaacgctggcctatcagttacattacaaaa

SEQ ID NO: 420 - DNA, 355 bp, Homo sapiens
  source 1..355: mol_type = genomic DNA
actacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgtttta

SEQ ID NO: 421 - DNA, 973 bp, Homo sapiens
  source 1..973: mol_type = genomic DNA
ccagctgctgctcttattgggctaagggatgaagactacacaggctgtcgagttctcctccaggatactcactgactcccaggtggcctactctatcaacccatgagcatggtcagccctgagacactgtcaggaagtgagaggccactgtaggagtgtcacgctaacagctaacccagcggcccccatgaaggctgacgcttggcatccctggagaatgggtcacacttttacctcatcgcatagccatagtctctgcctccacagccttcattttctcactttttctatcaatgcccaagaaaagaatgtttttccatgcaaaacccagcaaagaaataaagtccccaagcctgtgtgcagggttaaaggaacctcatgagagtgaagcactaggctgcgaaggggagaaacccacttgggaaaagcccaccacttcggggaacctggatgacggcccatagagcatagtgccacaaacagtgactctgaaagttcgtgttccatggttccaagatttgaagtagtgttactcctcagaggcctttgttactaatccccacggtgggctttttccaccacaggttttaacctagctttgaactatgttcaagtcctatgaggttgcaaagcaccaccccaatattcttatacctataacggggcacagaagctactgaagcatttggtagggctgtggctgcctgtgcagtgggctatgtgaaaatcaactggagtctcctggagagtgggctctggaagtctcctcagtcatcaacgcaggctcttgacctctcgtgcactatatgaccaaagtctacgcaggctatgtgggctatttgcgattcaagtccagctatgtgctgcttctaacccaggcagtattccagactgcagtccagctgagcgtggaatgtgtatcctggtgcccggattccacacttcactgccttggtgattcagccgaccctca

SEQ ID NO: 422 - DNA, 259 bp, Homo sapiens
  source 1..259: mol_type = genomic DNA
gcattttgctaagtccctgagggtcactggtcctcaaagcggcatggcggcatggcgtggctggttctgccacatgccagctgtgtgacctctgagactccacttcttcagtgctgaaaataaagaaggagttttactaaggaccaaacaagataatgaatgtgaaactgctccacgaaccccaaagaattatgcacatagatgcgatcattaagatgcgaagccatcgagttaccacctggcatgcttaaactgtaaa

SEQ ID NO: 423 - DNA, 77 bp, Homo sapiens
  source 1..77: mol_type = genomic DNA
ctgcggcggctcgcgggagacgctgcgcgcggggctagcgggcggcggagcggacggcgacggggcgctctcgggct

SEQ ID NO: 424 - DNA, 33 bp, Homo sapiens
  source 1..33: mol_type = genomic DNA
tccctgcctccctgcgtggtggactgtggcacc

SEQ ID NO: 425 - DNA, 27 bp, Homo sapiens
  source 1..27: mol_type = genomic DNA
aactatctccttcctgctgtaatcagt

SEQ ID NO: 426 - DNA, 48 bp, Homo sapiens
  source 1..48: mol_type = genomic DNA
aggctccaccatgttcagggatttcggacgccgactgcagagggattt

SEQ ID NO: 427 - DNA, 62 bp, Homo sapiens
  source 1..62: mol_type = genomic DNA
gaggtccaggtggtcacgcatcacatgcagcgctacgccgtgtggttcggaggctccatgct

SEQ ID NO: 428 - DNA, 61 bp, Homo sapiens
  source 1..61: mol_type = genomic DNA
aaggactatgaagagtacgggcccagcatctgccgccacaaccccgtctttggagtcatgt

SEQ ID NO: 429 - DNA, 107 bp, Homo sapiens
  source 1..107: mol_type = genomic DNA
ttcgatggtgtcacgttggggaacaagtgtccttcagaacccagagaaggccgccgttctgtaaatagcgacgtcggtgttgctgcccagcagcgtgcttgcattgc

SEQ ID NO: 430 - DNA, 194 bp, Homo sapiens
  source 1..194: mol_type = genomic DNA
ccatttatccgtgtgccgaccgctgtctgccagcctcctccttctcccgccctcctcaccctcgctctccctcctcctcctcctccgagctgctagctgacaaatacaattctgaaggaatccaaatgtgactttgaaaattgttagagaaaacaacattagaaaatggcgcaaaatcgttaggtcccaggaga

SEQ ID NO: 431 - DNA, 331 bp, Homo sapiens
  source 1..331: mol_type = genomic DNA
gtacaactggctgatactaagcacgaatagatattgatgttatggagtgctgtaatccaaagtttttaattgtgaggcatgttctgatatgtttataggcaaacaaataaaacagcaaacttttttgccacatgtttgctagaaaatgattatactttattggagtgacatgaagtttgaacactaaacagtaatgtatgagaattactacagatacatgtatcttttagttttttttgtttgaactttctggagctgttttatagaagatgatggtttgttgtcggtgagtgttggatgaaatacttccttgcaccattgtaataaaagc

SEQ ID NO: 432 - DNA, 33 bp, Homo sapiens
  source 1..33: mol_type = genomic DNA
attgacccagtcgacaggatctgaacttccagt

SEQ ID NO: 433 - DNA, 140 bp, Homo sapiens
  source 1..140: mol_type = genomic DNA
ggacctgttccaagctctcacgttccacatcacacatgggacatctagtgtcaggctcccagagagcaggaaccaggtgaaatataagagcacagtcctcccagccggtggcatggggataatcggacaatacaactctc

SEQ ID NO: 434 - DNA, 132 bp, Homo sapiens
  source 1..132: mol_type = genomic DNA
ccctgaacatggacttgcagaattccacagaagagaggagactggcctagacagacagccccaggagctgagggcccaacaggctttctaccctggatgctgctcccatgccctgacatgaggcccactaca

SEQ ID NO: 435 - DNA, 203 bp, Homo sapiens
  source 1..203: mol_type = genomic DNA
cagcctggatgtgaactgcaactccaaagtgtgtccagactcaaggcaagggcactaggctttccagacctcctactaagtcattgatccagcactgccctgccaggacataaatccctggcacctcttgctctctgcaaaggagggcaaagcagcttcaggagcccttgggagtcctccaaagagagtctagggtacaggtc

SEQ ID NO: 436 - DNA, 108 bp, Homo sapiens
  source 1..108: mol_type = genomic DNA
acagaagggccatgctgttattactcttacacaaggaggcagccctcgagccacagggtccagctgttggctataatagcctaccggtctctgatgatcaccatgttt

SEQ ID NO: 437 - DNA, 54 bp, Homo sapiens
  source 1..54: mol_type = genomic DNA
gcagcaatctgtcttctggattaaaactgaagatcaacctactttcaacttact

SEQ ID NO: 438 - DNA, 40 bp, Homo sapiens
  source 1..40: mol_type = genomic DNA
aaatatagccataattagcctactcaaatccaagtgtaaa

SEQ ID NO: 439 - DNA, 733 bp, Homo sapiens
  source 1..733: mol_type = genomic DNA
ccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgtttacagcactccagccaaaaaatacagcactggcatgattcaccttctcctgcaggtgaccattgatggcaggaactacattgtcgatgctgggtttggacgctcataccagatgtggcagcctctggagttaatttctgggaaggatcagcctcaggtgccttgtgtcttccgtttgacggaagagaatggattctggtatctagaccaaatcagaagggaacagtacattccaaatgaagaatttcttcattctgatctcctagaagacagcaaataccgaaaaatctactcctttactcttaagcctcgaacaattgaagattttgagtctatgaatacatacctgcagacatctccatcatctgtgtttactagtaaatcattttgttccttgcagaccccagatggggttcactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgataga

SEQ ID NO: 440 - DNA, 90 bp, Homo sapiens
  source 1..90: mol_type = genomic DNA
ataaggagtaaaacaatcttgtctatttgtcatccagctcaccagttatcaactgacgacctatcatgtatcttctgtacccttacctta

SEQ ID NO: 441 - DNA, 54 bp, Homo sapiens
  source 1..54: mol_type = genomic DNA
aaagatggcctgtggttatcttggaaattggtgatttatgctagaaagctttta

SEQ ID NO: 442 - DNA, 117 bp, Homo sapiens
  source 1..117: mol_type = genomic DNA
cttgtgtagatctgagttgaaatcctgtggacactgggcgaattactttttagatctgtagctctgactcctcaggcataaaatgggaataatgcttttacagtttagtggcggaac

SEQ ID NO: 443 - DNA, 182 bp, Homo sapiens
  source 1..182: mol_type = genomic DNA
taacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgc

SEQ ID NO: 444 - DNA, 43 bp, Homo sapiens
  source 1..43: mol_type = genomic DNA
atgcggtttgtcaaacagtactgctacggaggagcagcagaga

SEQ ID NO: 445 - DNA, 382 bp, Homo sapiens
  source 1..382: mol_type = genomic DNA
gtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttc

SEQ ID NO: 446 - DNA, 35 bp, Homo sapiens
  source 1..35: mol_type = genomic DNA
caacccttgccgcatccacgaaactttgcccatag

SEQ ID NO: 447 - DNA, 137 bp, Homo sapiens
  source 1..137: mol_type = genomic DNA
ttacaacacccgagcaaggacgcgactctcccgacgcggggaggctattctgcccatttggggacacttccccgccgctgccaggacccgcttctctgaaaggctctccttgcagctgcttagacgctggatttttt

SEQ ID NO: 448 - DNA, 387 bp, Homo sapiens
  source 1..387: mol_type = genomic DNA
agccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagc

SEQ ID NO: 449 - DNA, 284 bp, Homo sapiens
  source 1..284: mol_type = genomic DNA
gaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagc

SEQ ID NO: 450 - DNA, 139 bp, Homo sapiens
  source 1..139: mol_type = genomic DNA
ttccttctaacagaaatgtcctgagcaatcacctatgaacttgtttcaaatgcatgatcaaatgcaacctcacaaccttggctgagtcttgagactgaaagatttagccataatgtaaactgcctcaaattggactttg

SEQ ID NO: 451 - DNA, 38 bp, Homo sapiens
  source 1..38: mol_type = genomic DNA
tcctagtatatagtacctagtattataggtactataaa

SEQ ID NO: 452 - DNA, 566 bp, Homo sapiens
  source 1..566: mol_type = genomic DNA
tgtgcagctctctaaatgggaattctcaggtaggaagcaacagcttcagaaagagctcaaaataaattggaaatgtgaatcgcagctgtgggttttaccaccgtctgtctcagagtcccaggaccttgagtgtcattagttactttattgaaggttttagacccatagcagctttgtctctgtcacatcagcaatttcagaaccaaaagggaggctctctgtaggcacagagctgcactatcacgagcctttgtttttctccacaaagtatctaacaaaaccaatgtgcagactgattggcctggtcattggtctccgagagaggaggtttgcctgtgatttcctaattatcgctagggccaaggtgggatttgtaaagctttacaataatcattctggatagagtcctgggaggtccttggcagaactcagttaaatctttgaagaatatttgtagttatcttagaagatagcatgggaggtgaggattccaaaaacattttatttttaaaatatcctgtgtaacacttggctcttggtacctgtgggttagcatcaagttctcc

SEQ ID NO: 453 - DNA, 1528 bp, Homo sapiens
  source 1..1528: mol_type = genomic DNA
gcgcctgtcagtagtggacattgtaatccagtcggcttgttcttgcagcattcccgctcccttccctccatagccacgctccaaaccccagggtagccatggccgggtaaagcaagggccatttagattaggaaggtttttaagatccgcaatgtggagcagcagccactgcacaggaggaggtgacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtaatagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcagtagttttcaaaacacatagttaaaaaagaaacaaatgaaaaaaattttagaacagtccagcaaattgctagtcagggtgaattgtgaaattgggtgaagagcttaggattctaatctcatgttttttccttttcacatttttaaaagaacaatgacaaacacccacttatttttcaaggttttaaaacagtctacattgagcatttgaaaggtgtgctagaacaaggtctcctgatccgtccgaggctgcttcccagaggagcagctctccccaggcatttgccaagggaggcggatttccctggtagtgtagctgtgtggctttccttcctgaagagtccgtggttgccctagaacctaacaccccctagcaaaactcacagagctttccgtttttttctttcctgtaaagaaacatttcctttgaacttgattgcctatggatcaaagaaattcagaacagcctgcctgtccccccgcactttttacatatatttgtttcatttctgcagatggaaagttgacatgggtggggtgtccccatccagcgagagagtttcaaaagcaaaacatctctgcagtttttcccaagtaccctgagatacttcccaaagcccttatgtttaatcagcgatgtatataagccagttcacttagacaactttacccttcttgtccaatgtacaggaagtagttctaaaaaaaatgcatattaatttcttcccccaaagccggattcttaattctctgcaacactttgaggacatttatgattgtccctctgggccaatgcttatacccagtgagga

SEQ ID NO: 454 - DNA, 46 bp, Homo sapiens
  source 1..46: mol_type = genomic DNA
aatgaaaaaccatgagtgccccacctttcagtccgtgtttaagtga

SEQ ID NO: 455 - DNA, 57 bp, Homo sapiens
  source 1..57: mol_type = genomic DNA
cttcctcatcatgggccgcaaggtgaagagccagtacttgctgacggccatccacaa

SEQ ID NO: 456 - DNA, 76 bp, Homo sapiens
  source 1..76: mol_type = genomic DNA
aagttggggcccatcaagaagaaggacctgaagaagcttgtgctgtacctgaagaatggggctgactgtccctgcc

SEQ ID NO: 457 - DNA, 31 bp, Homo sapiens
  source 1..31: mol_type = genomic DNA
agaaaatggcgacaagaagattgtccccaag

SEQ ID NO: 458 - DNA, 69 bp, Homo sapiens
  source 1..69: mol_type = genomic DNA
tgtgtcctccctgtgacaacgagttgaaatctgaggccatcattgaacatctctgtgccagcgagtttg

SEQ ID NO: 459 - DNA, 219 bp, Homo sapiens
  source 1..219: mol_type = genomic DNA
gctcggctgcggaaaaggtgctgcagtccacagggaacatggggggattggacgggttgcctgaagaagagaggaaagaatccctcaccccagcccccaaaaggctgtgatgggatggggaaaccccataatcgctgtcttccggacaccttttgccccttggctgcagttccactggtcggcgcccttctcagcctggcttggaaccgtcctcactca

SEQ ID NO: 460 - DNA, 388 bp, Homo sapiens
  source 1..388: mol_type = genomic DNA
tcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgaca

SEQ ID NO: 461 - DNA, 28 bp, Homo sapiens
  source 1..28: mol_type = genomic DNA
gtcgcggagaacagggcgcagagccggc

SEQ ID NO: 462 - DNA, 27 bp, Homo sapiens
  source 1..27: mol_type = genomic DNA
tccctggaagtttgcggcaggacgcgc

SEQ ID NO: 463 - DNA, 210 bp, Homo sapiens
  source 1..210: mol_type = genomic DNA
tgcagcctccggagtcagtgccgcgcgcccgccgccccgcgccttcctgctcgccgcacctccgggagccggggcgcacccagcccgcagcgccgcctccccgcccgcgccgcctccgaccgcaggccgagggccgccactggccggggggaccgggcagcagcttgcggccgcggagccgggcaacgctggggactgcgccttttgtcc

SEQ ID NO: 464 - DNA, 175 bp, Homo sapiens
  source 1..175: mol_type = genomic DNA
aatggatccaactgcttgccccgtcatcccagatggctaggcccccattcatcccctctcgctctcctactggaggaactgctgtatgaatcataaagctctgggtagggaagcagggagcaggttccaggcagagctgacaagtgacttcacttttgagcatcggttgaaccag

SEQ ID NO: 465 - DNA, 100 bp, Homo sapiens
  source 1..100: mol_type = genomic DNA
aagcggccacaacccggcgatcgaaaagattcttaggaacgccgtaccagccgcgtctctcaggacagcaggcccctgtccttctgtcgggcgccgctca

SEQ ID NO: 466 - DNA, 103 bp, Homo sapiens
  source 1..103: mol_type = genomic DNA
cccagtttgctcctggctctcgggagactggaggatttcatcggagccccgcgctttaccagccctgttccctggataagatatttgacctttccgacccgcg

SEQ ID NO: 467 - DNA, 31 bp, Homo sapiens
  source 1..31: mol_type = genomic DNA
ttctaattccaaataaacttgcaagaggact

SEQ ID NO: 468 - DNA, 44 bp, Homo sapiens
  source 1..44: mol_type = genomic DNA
tgaaagattatgatgaacttctcaaatattatgaattacatgaa

SEQ ID NO: 469 - DNA, 55 bp, Homo sapiens
  source 1..55: mol_type = genomic DNA
aaaggtcaaacttgcctgccatatccttactggagagatggtagctataaaaatc

SEQ ID NO: 470 - DNA, 105 bp, Homo sapiens
  source 1..105: mol_type = genomic DNA
cccggatcaaaacggagattgaggccttgaagaacctgagacatcagcatatatgtcaactctaccatgtgctagagacagccaacaaaatattcatggttcttg

SEQ ID NO: 471 - DNA, 42 bp, Homo sapiens
  source 1..42: mol_type = genomic DNA
tactgccctggaggagagctgtttgactatataatttcccag

SEQ ID NO: 472 - DNA, 68 bp, Homo sapiens
  source 1..68: mol_type = genomic DNA
cagaagaggagacccgggttgtcttccgtcagatagtatctgctgttgcttatgtgcacagccagggc

SEQ ID NO: 473 - DNA, 78 bp, Homo sapiens
  source 1..78: mol_type = genomic DNA
acaaggattaccatctacagacatgctgtgggagtctggcttatgcagcacctgagttaatacaaggcaaatcatatc

SEQ ID NO: 474 - DNA, 40 bp, Homo sapiens
  source 1..40: mol_type = genomic DNA
gcagatgtttggagcatgggcatactgttatatgttctta

SEQ ID NO: 475 - DNA, 36 bp, Homo sapiens
  source 1..36: mol_type = genomic DNA
aagtggctctctcccagtagcattctgcttcttcaa

SEQ ID NO: 476 - DNA, 25 bp, Homo sapiens
  source 1..25: mol_type = genomic DNA
gacccaaagaaacggatttctatga

SEQ ID NO: 477 - DNA, 38 bp, Homo sapiens
  source 1..38: mol_type = genomic DNA
gcaagattacaactatcctgttgagtggcaaagcaaga

SEQ ID NO: 478 - DNA, 76 bp, Homo sapiens
  source 1..76: mol_type = genomic DNA
tttattcacctcgatgatgattgcgtaacagaactttctgtacatcacagaaacaacaggcaaacaatggaggatt

SEQ ID NO: 479 - DNA, 111 bp, Homo sapiens
  source 1..111: mol_type = genomic DNA
tggcagtatgatcacctcacggctacctatcttctgcttctagccaagaaggctcggggaaaaccagttcgtttaaggctttcttctttctcctgtggacaagccagtgct

SEQ ID NO: 480 - DNA, 104 bp, Homo sapiens
  source 1..104: mol_type = genomic DNA
tggaagatgtgaccgcaagtgataaaaattatgtggcgggattaatagactatgattggtgtgaagatgatttatcaacaggtgctgctactccccgaacatca

SEQ ID NO: 481 - DNA, 145 bp, Homo sapiens
  source 1..145: mol_type = genomic DNA
ggggtggaatctaaatcattaactccagccttatgcagaacacctgcaaataaattaaagaacaaagaaaatgtatatactcctaagtctgctgtaaagaatgaagagtactttatgtttcctgagccaaagactccagttaata

SEQ ID NO: 482 - DNA, 85 bp, Homo sapiens
  source 1..85: mol_type = genomic DNA
gaaaccagtgcctgaaagaaactccaattaaaataccagtaaattcaacaggaacagacaagttaatgacaggtgtcattagccc

SEQ ID NO: 483 - DNA, 146 bp, Homo sapiens
  source 1..146: mol_type = genomic DNA
tggatctcaaccaagcacatatggaggagactccaaaaagaaagggagccaaagtgtttgggagccttgaaagggggttggataaggttatcactgtgctcaccaggagcaaaaggaagggttctgccagagacgggcccagaaga

SEQ ID NO: 484 - DNA, 52 bp, Homo sapiens
  source 1..52: mol_type = genomic DNA
cttcactataacgtgactacaactagattagtgaatccagatcaactgttga

SEQ ID NO: 485 - DNA, 80 bp, Homo sapiens
  source 1..80: mol_type = genomic DNA
atacactgaagtgtcaaacacagtcagattttgggaaagtgacaatgcaatttgaattagaagtgtgccagcttcaaaaa

SEQ ID NO: 486 - DNA, 58 bp, Homo sapiens
  source 1..58: mol_type = genomic DNA
cttaagggcgatgcctgggtttacaaaagattagtggaagacatcctatctagctgca

SEQ ID NO: 487 - DNA, 206 bp, Homo sapiens
  source 1..206: mol_type = genomic DNA
atggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtc

SEQ ID NO: 488 - DNA, 75 bp, Homo sapiens
  source 1..75: mol_type = genomic DNA
gagggggcaccagtgatcgacacatttgcatctggaacgtgtgctctggggcctgtctgagtgccgtggatgccc

SEQ ID NO: 489 - DNA, 25 bp, Homo sapiens
  source 1..25: mol_type = genomic DNA
ttatttggaagtacccaaccatggc

SEQ ID NO: 490 - DNA, 261 bp, Homo sapiens
  source 1..261: mol_type = genomic DNA
cggacctatgtcctacctgttcatgcagttgtccatatccagtgggatgtcctaggggctcaactgtccacaaacatcactgccccccacagaactggtacagacccccaaaccacccatctcagcccatgaaaccactggccacccaagcccacaaatcagaaaccagtatgctgcctccttcccctcagcctttacatacagtcacataccaagcctgtcaattccactcccaatgtacctcctaaccatgagtctttg

SEQ ID NO: 491 - DNA, 85 bp, Homo sapiens
  source 1..85: mol_type = genomic DNA
accttctcaggagctgtagtgtaccattcagggacctggcacagtccatccagacatcctgctgagcgtcgcccacatatgcagt

SEQ ID NO: 492 - DNA, 340 bp, Homo sapiens
  source 1..340: mol_type = genomic DNA
tctttcaggtcctcagatgcactgcaccctctcctgcctgggggtctttgctcctgctactacctctgcttgaacagctcctcaccttccttcctccaaccctacccttgtataggtgacttttgttcatccttcagaattcaactcacatgtctcttgcatggagaaccctcacctactgtgttgagaccctgtccagcccccaggtgggatcctctctcgacttcccatacatttctttcacagcatttacatagtccatgatagtttacttgtgggattatttggttaatctttgcctttaacaccagggttccttgggtgaaggagcttctttatc

SEQ ID NO: 493 - DNA, 607 bp, Homo sapiens
  source 1..607: mol_type = genomic DNA
agagtcgttgaagtcccaggaattcaggactgggcaggttaagacctcagacaaggtagtagaggtagacttgtggacaaggctcgggtcccagcccaccgcaccccaactttaatcagagtggttcactattgatctatttttgtgtgatagctgtgtggcgtgggccacaacatttaatgagaagttactgtgcaccaaactgccgaacaccattctaaactattcatatatattagtcatttaattcttacataacttgagaggtagacagatatccttattttagagatgaggaaaccaagagaacttaggtcattagcgcaaggttgtagagtaagcggcaaagccaagacacaaagctgggtggtttggtttcagagccagtgcttttcccctctactgtactgcctctcaaccaacacagggttgcacaggcccattctctgatttttttcctcttgtcctctgcctctccctctagctcccacttcctctctgctctagttcattttctttagagcagcccgagtgatcatgaagtgcaaatcttgccatgtcagtcccctgcttagaaccctccaatggctcactttctctttaggca

SEQ ID NO: 494 - DNA, 210 bp, Homo sapiens
  source 1..210: mol_type = genomic DNA
cttctgtctctgtggttgtactgtccagcaatccaccttttctggagagggccacctctgcccaaattttcccagctgtttggacctctgggtgctttctttgggctggtgagagctctaatttgccttgggccagtttcaggtttataggccccctcagtcttcagatacatgagggcttctttgctcttgtgatcgtgtagtcccata

SEQ ID NO: 495 - DNA, 32 bp, Homo sapiens
  source 1..32: mol_type = genomic DNA
tttcctactctcacactggttctcaatgaaaa

SEQ ID NO: 496 - DNA, 83 bp, Homo sapiens
  source 1..83: mol_type = genomic DNA
aatggcgccaccagctctgccgtctctggagcggaatttacctgatttcttcagggctgctgggggcaactggccatttgcca

SEQ ID NO: 497 - DNA, 31 bp, Homo sapiens
  source 1..31: mol_type = genomic DNA
ttcaaagacagtagattgaaaaggaaaggct

SEQ ID NO: 498 - DNA, 55 bp, Homo sapiens
  source 1..55: mol_type = genomic DNA
gaagctctctgcaaacttgataggagagtaaaagccacaatagagcagtttatga

SEQ ID NO: 499 - DNA, 638 bp, Homo sapiens
  source 1..638: mol_type = genomic DNA
agctcttgctacgtgttgtgaaatctgtgtctgtagctcgtggctgtgataaacatcctcagagatccctgctatccaactcttgtcaccagctaactggtcattaggccaagctgtgtgagtgaaccattagacaatttgataaaaataagcactgctgccattctttagcatgcgtctgtgccacaagttagtgacacatgtacatatattccctttacctcacagtaaccatctagggtgtgggcatcataatcttcatttttaaaatgagggtactgaggcatgaataggctaaataatttgctcaagatcacaagttagtaaatggcagtcagttttcaggcgtaggctttgaagactctaaacctgtgcttttaacatctgcctcttcttgtgactgggggcttcactggagccaagcctcaggaggcatttgcagccacagtgggtcatgcgaggtagagcagactgcaacctgatagaattcttggactggcccacggccagcctttttaatttttccaagagttaaagttgtggatctgagatgtggcctggcctgccagggctatggtgggctcagtgtgattgcatcattagtattgtggcatggagtcttccgtcagcctcagaga

SEQ ID NO: 500 - DNA, 110 bp, Homo sapiens
  source 1..110: mol_type = genomic DNA
agtccacaggaagaggttgaactaaagaagttgaaacatttggagaagtctgtggagaagatagctgaccagctggaagagttgaataaagagcttactggaatccagca

SEQ ID NO: 501 - DNA, 618 bp, Homo sapiens
  source 1..618: mol_type = genomic DNA
ctaaatttcttgcttcagggtcagggagaatggtgatgggcaacaggcagaaccaaagagtcaagtttgaagaaactataggacttttacttggagctgacacagagcgtatggagcgaagtgtgaactcaaggaaggcaccatggcacatgcctattgtcccagcaagttgggagtctgagacaggaggatcacttgagtccaggggtttgaggctgcagtgagttatgatcatgcctgtaaacagccactgcactccaacctgggaaccgtagtgagaccctatctcaaaaaagaaaaaagtctgaacttaagtctaatctacctcttttggactgtgtgatctcatgttactttactacattaagcctcagtttcatcatctgtaaaacagcagtacttccctgatggagttgtggtaaggcttaaaaaataggtaaggtgcttaggatagtgtgtggcatgtaggaagtgttcaataaaagtattcatcgttgttaaccagcatcacattaaacaggagcgctcaattggaggctgccatttaggaattccatttaaaggaatggtagaattccacctttcttgccattctggactcctcacaagtgtttactg

SEQ ID NO: 502 - DNA, 51 bp, Homo sapiens
  source 1..51: mol_type = genomic DNA
ggaaatggaaacaccgttgtcagcacttggaatacaagatggttgccgggt

SEQ ID NO: 503 - DNA, 110 bp, Homo sapiens
  source 1..110: mol_type = genomic DNA
gacctgctaggtattggacactctcaaacacctggacaggtgtcagatttgaagtcttatgatgatggcagaaatctcaccattttcgatgaagctcatgggcagtcttc

SEQ ID NO: 504 - DNA, 106 bp, Homo sapiens
  source 1..106: mol_type = genomic DNA
gagaagcacgaccttcatgttacctcccagcagggcagcagtgaaccagttgtccaagacctggcccaggttgttgaagaggtcataggggttccacagtcttttc

SEQ ID NO: 505 - DNA, 31 bp, Homo sapiens
  source 1..31: mol_type = genomic DNA
ggcagctgggctcaccgtgactgtcacccac

SEQ ID NO: 506 - DNA, 42 bp, Homo sapiens
  source 1..42: mol_type = genomic DNA
gaggcgacccagggcgaagagatgaatcggagccaggaggtg

SEQ ID NO: 507 - DNA, 53 bp, Homo sapiens
  source 1..53: mol_type = genomic DNA
agttgaccctgagtgaggaagcgacctggagtgaagaggcgacccagagtgag

SEQ ID NO: 508 - DNA, 29 bp, Homo sapiens
  source 1..29: mol_type = genomic DNA
acccggcgccgctcgacccggagcgagga

SEQ ID NO: 509 - DNA, 60 bp, Homo sapiens
  source 1..60: mol_type = genomic DNA
tgggcgtccacctgcccggagtactgccagcgggcatgaccgacccaccaggggcgccgc

SEQ ID NO: 510 - DNA, 31 bp, Homo sapiens
  source 1..31: mol_type = genomic DNA
ctgggttcccggctgcgcgcccttcggccag

SEQ ID NO: 511 - DNA, 55 bp, Homo sapiens
  source 1..55: mol_type = genomic DNA
gcggggttgtgagacgccgcgctcagcttccatcgctgggcggtcaacaagtgcg

SEQ ID NO: 512 - DNA, 43 bp, Homo sapiens
  source 1..43: mol_type = genomic DNA
tgtgactcggccgacgcgagcgccgcgcttcgcttcagctgct

SEQ ID NO: 513 - DNA, 47 bp, Homo sapiens
  source 1..47: mol_type = genomic DNA
ggaggcgaccgcggagggtggcgaggggcggccaggacccgcagccc

SEQ ID NO: 514 - DNA, 133 bp, Homo sapiens
  source 1..133: mol_type = genomic DNA
ggcagatcgcgtccgcgggattcaatctctgcccgctctgataacagtccttttccctggcgctcacttcgtgcctggcacccggctgggcgcctcaagaccgttgtctcttcgatcgcttctttggacttgg

SEQ ID NO: 515 - DNA, 31 bp, Homo sapiens
  source 1..31: mol_type = genomic DNA
atgtcttccagaagtaccaaagatttaatta

SEQ ID NO: 516 - DNA, 142 bp, Homo sapiens
  source 1..142: mol_type = genomic DNA
taagtggggatcgaagcctagtaactccaaatccgaaactacattagaaaaattaaagggagaaattgcacacttaaagacatcagtggatgaaatcacaagtgggaaaggaaagctgactgataaagagagacacagactt

SEQ ID NO: 517 - DNA, 157 bp, Homo sapiens
  source 1..157: mol_type = genomic DNA
gcgactgagagaccaactgaaggccagatatagtactaccacattgcttgaacagctggaagagacaacgagagaaggagaaaggagggagcaggtgttgaaagccttatctgaagagaaagacgtattgaaacaacagttgtctgctgcaacctca

SEQ ID NO: 518 - DNA, 38 bp, Homo sapiens
  source 1..38: mol_type = genomic DNA
ctgtggctccaaactgcttcaactcatcaataaataat

SEQ ID NO: 519 - DNA, 137 bp, Homo sapiens
  source 1..137: mol_type = genomic DNA
tctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagc

SEQ ID NO: 520 - DNA, 76 bp, Homo sapiens
  source 1..76: mol_type = genomic DNA
gaagcagaaatgttacaacgatctcttggcaagtgcaaaaaaagatcttgaggttgaacgacaaaccataactcag

SEQ ID NO: 521 - DNA, 150 bp, Homo sapiens
  source 1..150: mol_type = genomic DNA
agctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctc

SEQ ID NO: 522 - DNA, 63 bp, Homo sapiens
  source 1..63: mol_type = genomic DNA
ctttacacatctctgctaaagcagcaagaagaacaaacaagggtagctctgttggaacaacag

SEQ ID NO: 523 - DNA, 108 bp, Homo sapiens
  source 1..108: mol_type = genomic DNA
actcattcgggttgcttctaaattcaattctgcctgttagaaaatgcagtttttcctcatgtttatgctgttctatggagaactgtttgaaagttgtgaaaagtgtct

SEQ ID NO: 524 - DNA, 97 bp, Homo sapiens
  source 1..97: mol_type = genomic DNA
tgaaaaactcgaccgtcaacatgtgcagcatcaattgcatgtaattcttaaggagctccgaaaagcaagaaatcaaataacacagttggaatccttg

SEQ ID NO: 525 - DNA, 143 bp, Homo sapiens
  source 1..143: mol_type = genomic DNA
tccgaacttaggaggatacagcttaacacacagctagctgtatctcaaatcagtaggtagagcctctgcctcatttgaagcaactgccctttgagcatcaattcagaggacatgaaagagggacatgatcacatctggaaaca

SEQ ID NO: 526 - DNA, 159 bp, Homo sapiens
  source 1..159: mol_type = genomic DNA
cagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatg

SEQ ID NO: 527 - DNA, 494 bp, Homo sapiens
  source 1..494: mol_type = genomic DNA
tacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggttttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctg

SEQ ID NO: 528 - DNA, 116 bp, Homo sapiens
  source 1..116: mol_type = genomic DNA
tcagatctttgtttgtctgaacaggtatttttatacatgctttttgtaaaccaaaaacttttaaatttcttcaggttttctaacatgcttaccactgggctactgtaaatgagaaa

SEQ ID NO: 529 - DNA, 1377 bp, Homo sapiens
  source 1..1377: mol_type = genomic DNA
actcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaaca

SEQ ID NO: 530 - DNA, 115 bp, Homo sapiens
  source 1..115: mol_type = genomic DNA
atgagcgcacggatgaatggagcttacaagatctgtctttccaatggccgggggcatttggtccccaaattaaggctattggacatctgcacaggacagtcctatttttgatgtc

SEQ ID NO: 531 - DNA, 50 bp, Homo sapiens
  source 1..50: mol_type = genomic DNA
aggaacaactatcctcgtctgtcccaacactgagcaggcactcggtaaac

SEQ ID NO: 532 - DNA, 227 bp, Homo sapiens
  source 1..227: mol_type = genomic DNA
atttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctcc

SEQ ID NO: 533 - DNA, 60 bp, Homo sapiens
  source 1..60: mol_type = genomic DNA
ggacaatgtgtgtgtcaagaaaataagaaccagaagtcatagggacagtgaagatatttg

SEQ ID NO: 534 - DNA, 155 bp, Homo sapiens
  source 1..155: mol_type = genomic DNA
cagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaa

SEQ ID NO: 535 - DNA, 30 bp, Homo sapiens
  source 1..30: mol_type = genomic DNA
gagcccggaaacccatacctagagacaaag

SEQ ID NO: 536 - DNA, 26 bp, Homo sapiens
  source 1..26: mol_type = genomic DNA
tatccctgcgctccagacgccaaaat

SEQ ID NO: 537 - DNA, 226 bp, Homo sapiens
  source 1..226: mol_type = genomic DNA
aagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctg

SEQ ID NO: 538 - DNA, 1286 bp, Homo sapiens
  source 1..1286: mol_type = genomic DNA
cccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgta

SEQ ID NO: 539 - DNA, 3489 bp, Homo sapiens
  source 1..3489: mol_type = genomic DNA
ggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaa

SEQ ID NO: 540 - DNA, 389 bp, Homo sapiens
  source 1..389: mol_type = genomic DNA
aacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctc

SEQ ID NO: 541 - DNA, 334 bp, Homo sapiens
  source 1..334: mol_type = genomic DNA
tgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggc

SEQ ID NO: 542 - DNA, 62 bp, Homo sapiens
  source 1..62: mol_type = genomic DNA
cccagtgaaggagcaaccgcagttgacaagcacatgtcacatcgctatttcaaattcagaga

SEQ ID NO: 543 - DNA, 90 bp, Homo sapiens
  source 1..90: mol_type = genomic DNA
taaaaacgtagtcttagatcttataaatcttttgactctactgttttttactgtgttaatgtttgttttgctaactttgtttatctgctg

SEQ ID NO: 544 - DNA, 84 bp, Homo sapiens
  source 1..84: mol_type = genomic DNA
cgcaaactctccttgtaccataataatagggaaagctcatactgaaaaagtacatgtgcctgctcgaccctacagagtgctcaa

SEQ ID NO: 545 - DNA, 26 bp, Homo sapiens
  source 1..26: mol_type = genomic DNA
agcctgtgggcgaagttcacagtcaa

SEQ ID NO: 546 - DNA, 73 bp, Homo sapiens
  source 1..73: mol_type = genomic DNA
catgggcagatgtagtaaaacttggtgcaaaacaaacacaaactaaagtcataaaacatggtcctcaaaggtc

SEQ ID NO: 547 - DNA, 84 bp, Homo sapiens
  source 1..84: mol_type = genomic DNA
aagagagtgtctatcagccgaagtcaacatgatattttacagatgatatgttccaaaagaagaagtggtgcttcggaagcaaat

SEQ ID NO: 548 - DNA, 116 bp, Homo sapiens
  source 1..116: mol_type = genomic DNA
aaacaagagtcaggttcagaaatccatgtggaagtgaaggcacaaagcttggttataagccctccagctcctagtcctaggaaaactccagttgccagtgatcaacgccgtaggtc

SEQ ID NO: 549 - DNA, 134 bp, Homo sapiens
  source 1..134: mol_type = genomic DNA
cctttgaaaagaaggcgtgtgtcctttggtgggcacctaagacctgaactatttgatgaaaacttgcctcctaatacgcctctcaaaaggggagaagccccaaccaaaagaaagtctctggtaatgcacactcc

SEQ ID NO: 550 - DNA, 66 bp, Homo sapiens
  source 1..66: mol_type = genomic DNA
ggacagatgtgctctgggttacctggtcttagttcagttgatatcaacaactttggtgattccatt

SEQ ID NO: 551 - DNA, 31 bp, Homo sapiens
  source 1..31: mol_type = genomic DNA
ttgagaggaagatccaaaaggattccctcag

SEQ ID NO: 552 - DNA, 149 bp, Homo sapiens
  source 1..149: mol_type = genomic DNA
gaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagc

SEQ ID NO: 553 - DNA, 416 bp, Homo sapiens
  source 1..416: mol_type = genomic DNA
ccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatg

SEQ ID NO: 554 - DNA, 33 bp, Homo sapiens
  source 1..33: mol_type = genomic DNA
agccagcacgtcgtgtctcaagatctagcttct

SEQ ID NO: 555 - DNA, 30 bp, Homo sapiens
  source 1..30: mol_type = genomic DNA
ttcagaatggaaggaagtcaactgaatttc

SEQ ID NO: 556 - DNA, 86 bp, Homo sapiens
  source 1..86: mol_type = genomic DNA
ccaacacaagtaaatgggtctgttattgatgagcctgtacggctaaaacatggagatgtaataactattattgatcgttccttcag

SEQ ID NO: 557 - DNA, 43 bp, Homo sapiens
  source 1..43: mol_type = genomic DNA
tgtgacatccgtatccagcttcctgttgtgtcaaaacaacatt

SEQ ID NO: 558 - DNA, 30 bp, Homo sapiens
  source 1..30: mol_type = genomic DNA
cctgagcctcagcacctgcttgtttggaag

SEQ ID NO: 559 - DNA, 26 bp, Homo sapiens
  source 1..26: mol_type = genomic DNA
cccacgagacgcctggttactatcaa

SEQ ID NO: 560 - DNA, 72 bp, Homo sapiens
  source 1..72: mol_type = genomic DNA
tttgcttctggccttcccctacggattatacctggccttcccctacggattatactcaacttactgtttaga

SEQ ID NO: 561 - DNA, 265 bp, Homo sapiens
  source 1..265: mol_type = genomic DNA
gactcggtgggagccgctagagccgggcgcccggggacgtagcctgtagggccaccgggtccccgtcagaggcggcggcgggagcagcggggactgcaggccggggtgcagcgaacgcgaccccgcgggctgcggcccggtgtgtgcggagcgtggcgggcgcagcttaccgggcggaggtgagcgcggcgccggctcctcctgcggcggactttgggtgcgacttgacgagcggtggttcgacaagtggccttgcgggccggat

SEQ ID NO: 562 - DNA, 25 bp, Homo sapiens
  source 1..25: mol_type = genomic DNA
tgcagacggctgcgaggcgctgggc

SEQ ID NO: 563 - DNA, 30 bp, Homo sapiens
  source 1..30: mol_type = genomic DNA
gcagggctgagactatcttctgctcaggaa

SEQ ID NO: 564 - DNA, 62 bp, Homo sapiens
  source 1..62: mol_type = genomic DNA
tctcatgtttttctataagcagttaagagaagccacacagcatcctgaacactttgctttct

SEQ ID NO: 565 - DNA, 80 bp, Homo sapiens
  source 1..80: mol_type = genomic DNA
atggcaaatttcaagggccacgcgcttccagggagtttcttcctgatcattgggctgtgttggtcagtgaagtacccgct

SEQ ID NO: 566 - DNA, 46 bp, Homo sapiens
  source 1..46: mol_type = genomic DNA
gtactttagccacacgcggaagaacagcccactacattactatcag

SEQ ID NO: 567 - DNA, 49 bp, Homo sapiens
  source 1..49: mol_type = genomic DNA
cgtctcgagatcgtcgaagccgcaattaggactttgttttccgtcactg

SEQ ID NO: 568 - DNA, 192 bp, Homo sapiens
  source 1..192: mol_type = genomic DNA
gtcatttggtctagggaatctcctcatcatacccagaacctttaattcattttctgagccctgtgaaatagatgttcccactggcagagataatagggcaacaatttcctgatggccactagactattttatcgtaacatccattgtgtacagagctttataatactaacggttgacagctctcacatcatg

SEQ ID NO: 569 - DNA, 74 bp, Homo sapiens
  source 1..74: mol_type = genomic DNA
caccatgtacctattctttgcagtctcaggaattgttgacatgctcacctatctggtcagccacgttcccttgg

SEQ ID NO: 570 - DNA, 132 bp, Homo sapiens
  source 1..132: mol_type = genomic DNA
aaccggcctccgctggaccagcacatccactcactcctgctgtatgctctgttcggagggtgtgttagtatctccctagaggtgatcttccgggaccacattgtgctggaacttttccgaaccagtctcatc

SEQ ID NO: 571 - DNA, 139 bp, Homo sapiens
  source 1..139: mol_type = genomic DNA
attgggtttgtgctgttcccaccttttggaacacccgaatgggaccagaaggatgatgccaacctcatgttcatcaccatgtgcttctgctggcactacctggctgccctcagcattgtggccgtcaactattctcttg

SEQ ID NO: 572 - DNA, 102 bp, Homo sapiens
  source 1..102: mol_type = genomic DNA
ccttttgactcggatgaagagacacggaaggggagaaatcattggaattcagaagctgaattcagatgacacttaccagaccgccctcttgagtggctcaga

SEQ ID NO: 573 - DNA, 1032 bp, Homo sapiens
  source 1..1032: mol_type = genomic DNA
taaagtctgtgttggtatagtacccttcataaggaaaaatgaagtaatgcctataagtagcaggcctttgtgcctcagtgtcaagagaaatcaagagatgctaaaagctttacaatggaagtggcctcatggatgaatccggggtatgagcccaggagaacgtgctgcttttggtaacttatccctttttctcttaagaaagcaggtactttcttattagaaatatgttagaatgtgtaagcaaacgacagtgcctttagaattacaattctaacttacatattttttgaaagtaaaataattcacaagctttggtattttaaaattattgttaaacatatcataactaatcataccagggtactgcaataccactgtttataagtgacaaaattaggccaaaggtgatttttttttaaatcaggaagctggttactggctctactgagagttggagccctgatgttctgattcttcaaagtcaccctaaaagaagatctgacaggaaagctgtataatgagatagaaaaacgtcaggtatggaaggctttcagttttaatatggctgaaagcaaaggataacgaattcagaattagtaatgtaaaatcttgataccctaatcttgcttctggatctgttctttttttaaaaaaacttccttcaccgcgcctataatcctagcactttgggaggccgaggcaggcagatcacggggtcaggagatcaagaccatcctggctaacatggtgaaaccccgtctctactgaaaatacaaaaaattagccgggtgtggtggcgggcgcctgtagttccagctactcgggaggctgaggcaagagaatggcatgaacccggtaggggagcttgcagtgagcccagatcatgccactgtactccagcctaggtgacagagcaagactctgtctcaaaaacaagcaaacagacttccttcaacaaatatttattaaatatccactttgcaacagcactgaaatggctgtaaggactcctgagatatgtgtccagcaagg

SEQ ID NO: 574 - DNA, 2385 bp, Homo sapiens
  source 1..2385: mol_type = genomic DNA
agtgttcccgtcttctccgagcttagagttggatggggaataaagacaggtaaacagatagctacaatattgtactgtgaatgcttatgctggaggaagtacagggaactattggagcacctaagaggagcacctaccttgaatttaggggttagcagaggcatcctgaaaaaagtcaaagctaagccacaatctataagcagtttaggaattagcagaacgtgcgtggtgaggagatgccaaaggcaagaagagaagagtattccaaacaggagggattccaaagagagaagagtatcccaaacaacatttgcacaaacctgatggggagagagaatgtggggtggggatggatgatgagactgaagaagaaagccaggtctagataatcagtggccttgtacaccatgttaaagagtgtagacttgattctgttgtaaacaggaaagcagcacaattcatatgaatattttagaagactcccactggaatatggagaataaagttggagatgactaatcctggaagcagggagaacatttttgaggaagttgcactattttggtgaaaatgatgatcataaacatgaagaattgtaggtgatcatgacctcctctctaattttccagaagggttttggaagatataacataggaacattgacaggactgacgaaaggagatgaaatacaccatataaattgtcaaacacaaggccagatgtctaattattttgcttatgtgttgaaattacaaatttttcatcaggaaaccaaaaactacaaaacttagttttcccaagtcccagaattctatctgtccaaacaatctgtaccactccacctatatccctacctttgcatgtctgtccaacctcaaagtccaggtctatacacacgggtaagactagagcagttcaagtttcagaaaatgagaaagaggaactgagttgtgctgaacccatacaaaataaacacattctttgtatagattcttggaacctcgagaggaattcacctaactcataggtatttgatggtatgaatccatggctgggctcggcttttaaaaagccttatctgggattccttctatggaaccaagttccatcaaagcccatttaaaagcctacattaaaaacaaaattcttgctgcattgtatacaaataatgatgtcatgatcaaataatcagatgccattatcaagtggaattacaaaatggtatacccactccaaaaaaaaaaaaaaagctaaattctcagtagaacattgtgacttcatgagccctccacagccttggagctgaggagggagcactggtgagcagtaggttgaagagaaaacttggcgcttaataatctatccatgttttttcatctaaaagagccttctttttggattaccttattcaatttccatcaaggaaattgttagttccactaaccagacagcagctgggaaggcagaagcttactgtatgtacatggtagctgtgggaaggaggtttctttctccaggtcctcactggccatacaccagtcccttgttagttatgcctggtcatagacccccgttgctatcatctcatatttaagtctttggcttgtgaatttatctattctttcagcttcagcactgcagagtgctgggactttgctaacttccatttcttgctggcttagcacattcctcataggcccagctcttttctcatctggccctgctgtggagtcaccttgccccttcaggagagccatggcttaccactgcctgctaagcctccactcagctgccaccacactaaatccaagcttctctaagatgttgcagactttacaggcaagcataaaaggcttgatcttcctggacttccctttacttgtctgaatctcacctccttcaactttcagtctcagaatgtaggcatttgtcctctttgccctacatcttccttcttctgaatcatgaaagcctctcacttcctcttgctatgtgctggaggcttctgtcaggttttagaatgagttctcatctagtcctagtagcttttgatgcttaagtccaccttttaaggatacctttgagatttagaccatgtttttcgcttgagaaagccctaatctccagacttgcctttctgtggatttcaaagaccaactgaggaagtcaaaagctgaatgttgactttctttgaacatttccgctataacaattccaattctcctcagagcaatatgcctgcctccaactgaccaggagaaaggtccagtgccaaagagaaaaacacaaagattaattatttcagttgagcacatactttcaaagtggtttgggtattcatatgaggttttctgtcaagagggtgagactcttcatctatccatg

SEQ ID NO: 575 - DNA, 510 bp, Homo sapiens
  source 1..510: mol_type = genomic DNA
gccttgtaagtagccatggaatgtcaacctgtaacttaaattatccacagatagtcatgtgtttgatgatgggcactgtggagataactgacataggactgtgccccccttctctgccacttactagctggatgagattaagcaagtcatttaactgctctgattaaacctgcctttcccaagtgctttgtaatgaatagaaatggaaaccaaaaaaaacgtatacaggccttcagaaatagtaattgctactattttgttttcattaagccatagttctggctataattttatcaaactcaccagctatattctacagtgaaagcaggattctagaaagtctcactgttttatttatgtcaccatgtgctatgatatatttggttgaattcatttgaaattagggctggaagtattcaagtaatttcttctgctgaaaaaatacagtgttttgagtttagggcctgttttatcaaagttctaaagagcctatcactcttccattgtaga

SEQ ID NO: 576 - DNA, 253 bp, Homo sapiens
  source 1..253: mol_type = genomic DNA
gtcctatctactaatgtctccattactatttagtcatcataaccattatcttcattttacatgtcgtgttctttctggtagctctaaaatgacactaaatcataagaagacaggttacatatcaggaaatacttgaaggttactgaaatagattcttgagttaatgaaaatattttctgtaaaaaggtttgaaaagccatttgagtctaaagcattatacctccattatcagtagttatgtgacaattgtgtg

SEQ ID NO: 577 - DNA, 1054 bp, Homo sapiens
  source 1..1054: mol_type = genomic DNA
gcagtaatttgccttctcctagagtttacctgccattttgtgcacatttgagttacagtagcatgttattttacaattgtgactctcctgggagtctgggagccatataaagtggtcaatagtgtttgctgactgagagttgaatgacattttctctctgtcttggtattactgtagatttcgatcattctttggttacatttctgcatatttctgtacccatgactttatcactttcttctcccatgctttatctccatcaattatcttcattacttttaaattttccacctttgcttcctactttgtgagatctctccctttactgactataacatagaagaatagaagtgtattttatgtgtcttaaggacaatactttagattccttgttctaagtttttaaactgaatgaatggaatattatttctctccctaagcaaaattccacaaaacaattatttcttatgtttatgtagccttaaattgttttgtactgtaaacctcagcataaaaactttcttcatttctaatttcattcaacaaatattgattgaatacctggtattagcacaagaaaaatgtgctaataagccttatgagaatttggagctgaagaaagacatataactcaggaaagttacagtccagtagtaggtataaattacagtgcctgataaataggcattttaatatttgtacactcaacgtatactaggtaggtgcaaaacatttacatataattttactgatacccatgcagcacaaaggtactaactttaaatattaaataacacctttatgtgtcagtaattcatttgcattaaatcttattgaaaaggctttcaatatattttccccacaaatgtcatcccaagaaaaaagtatttttaacatctcccaaatataatagttacaggaaatctacctctgtgagagtgacacctctcagaatgaactgtgtgacacaagaaaatgaatgtaggtctatccaaaaaaaaccccaagaaacaaaaacaatattattagccctttatgcttaagtgatggactc

SEQ ID NO: 578 - DNA, 359 bp, Homo sapiens
  source 1..359: mol_type = genomic DNA
taggtaactccctttgtgtcaattatatttccaaaaatgaacctttaaaatggtatgcaaaattttgtctatatatatttgtgtgaggaggaaattcataactttcctcagattttcaaaagtatttttaatgcaaaaaatgtagaaagagtttaaaaccactaaaatagattgatgttcttcaaactaggcaaaacaactcatatgttaagaccattttccagattggaaacacaaatctcttaggaagttaataagtagattcatatcattatgcaaatagtattgtgggttttgtaggtttttaaaataaccttttttggggagagaattgtcctctaatgaggtattgcgagtgg

SEQ ID NO: 579 - DNA, 112 bp, Homo sapiens
  source 1..112: mol_type = genomic DNA
caaacaacttcatctgtactgcttgaatacatttatccagtcccgggcactgagtgttgaatttccagaaatgatgtctgaagttattgctgcacaattacccaagatattg

SEQ ID NO: 580 - DNA, 91 bp, Homo sapiens
  source 1..91: mol_type = genomic DNA
atgaggtcaagctacattagagagctcatcaaggcaattggtttgaggcaaaaaggagttgtgtcgagctcacagcgtttctatcaactta

SEQ ID NO: 581 - DNA, 25 bp, Homo sapiens
  source 1..25: mol_type = genomic DNA
tggaagggctacgaagtcaaaccca

SEQ ID NO: 582 - DNA, 33 bp, Homo sapiens
  source 1..33: mol_type = genomic DNA
caggagtttgtcaagcttcaagttagccaagaa

SEQ ID NO: 583 - DNA, 29 bp, Homo sapiens
  source 1..29: mol_type = genomic DNA
tattttgcacctgatctaatactaaatga

SEQ ID NO: 584 - DNA, 74 bp, Homo sapiens
  source 1..74: mol_type = genomic DNA
cttacatattgatgaccagataactctcattcagtattcttggatgagcttaatggtgtttggtctaggatgga

SEQ ID NO: 585 - DNA, 229 bp, Homo sapiens
  source 1..229: mol_type = genomic DNA
cagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtcta

SEQ ID NO: 586 - DNA, 46 bp, Homo sapiens
  source 1..46: mol_type = genomic DNA
atggagtttatatatatttacatgaatttcttttttttcttctctg

SEQ ID NO: 587 - DNA, 373 bp, Homo sapiens
  source 1..373: mol_type = genomic DNA
agaccattggcgtaccaccagtttgtggagggaactggaaaaactggaatacacatgccccatccaaaagcaaccattgcaactaaactttaacagattgttgccacctaagtaattcacggatggtctcataattctggtcagcattgtctgagccaaacaaaatgtatctatgggcatgatcagatactagagccagcagattgcaacctctgcttagataattgcaggtatcagccttcccttggctaaacagctacttcatactgataagtagcccttgcctggcacaaagcaggtggggctgaatccagcctgatatcacatcaccacaactttctctaattctcctcaaggcgtctgtgaactacca

SEQ ID NO: 588 - DNA, 63 bp, Homo sapiens
  source 1..63: mol_type = genomic DNA
tgactgcatcgttgataaaatccgcagaaaaaactgcccagcatgtcgccttagaaagtgctg

SEQ ID NO: 589 - DNA, 65 bp, Homo sapiens
  source 1..65: mol_type = genomic DNA
caggctgtcattatggtgtccttacctgtgggagctgtaaggtcttctttaagagggcaatggaa

SEQ ID NO: 590 - DNA, 51 bp, Homo sapiens
  source 1..51: mol_type = genomic DNA
agccagagcccacaatacagcttcgagtcattacctcagaagatttgttta

SEQ ID NO: 591 - DNA, 69 bp, Homo sapiens
  source 1..69: mol_type = genomic DNA
tcggctaccaggccgccgtgctcaaggagggcctgccgcaggtctacccgccctatctcaactacctga

SEQ ID NO: 592 - DNA, 130 bp, Homo sapiens
  source 1..130: mol_type = genomic DNA
gccgccctgcaaggcgccgggcgcgagcggctgcctgctcccgcgggacggcctgccctccacctccgcctctgccgccgccgccggggcggcccccgcgctctaccctgcactcggcctcaacgggctc

SEQ ID NO: 593 - DNA, 71 bp, Homo sapiens
  source 1..71: mol_type = genomic DNA
gtgcctcagtctcgtctgcgtcctcctcggggtcgaccctggagtgcatcctgtacaaagcggagggcgcg

SEQ ID NO: 594 - DNA, 241 bp, Homo sapiens
  source 1..241: mol_type = genomic DNA
ctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaagg

SEQ ID NO: 595 - DNA, 781 bp, Homo sapiens
  source 1..781: mol_type = genomic DNA
ttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatcc

SEQ ID NO: 596 - DNA, 261 bp, Homo sapiens
  source 1..261: mol_type = genomic DNA
tgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgcc

SEQ ID NO: 597 - DNA, 147 bp, Homo sapiens
  source 1..147: mol_type = genomic DNA
cgcaccgaggcaccgcggcgagcttggctgcttctggggcctgtgtggccctgtgtgtcggaaagatggagcaagaagccgagcccgaggggcggccgcgacccctctgaccgagatcctgctgctttcgcagccaggagcaccgtc

SEQ ID NO: 598 - DNA, 35 bp, Homo sapiens
  source 1..35: mol_type = genomic DNA
gtacgagcgcccagtgccctggcccggagagtgga

SEQ ID NO: 599 - DNA, 34 bp, Homo sapiens
  source 1..34: mol_type = genomic DNA
gaggcccagggcgtcgtgcttccgcgcgccccgt

SEQ ID NO: 600 - DNA, 35 bp, Homo sapiens
  source 1..35: mol_type = genomic DNA
actccaagcgcgaaaaccccggatggtgaggagca

SEQ ID NO: 601 - DNA, 57 bp, Homo sapiens
  source 1..57: mol_type = genomic DNA
ttcagtggcgattggagggtagacctgtgggcacggacgcacgccactttttctctg

SEQ ID NO: 602 - DNA, 30 bp, Homo sapiens
  source 1..30: mol_type = genomic DNA
tgtacctactgatggtgctgtaaccacctc

SEQ ID NO: 603 - DNA, 30 bp, Homo sapiens
  source 1..30: mol_type = genomic DNA
accaaagccattgcttttgaagttattaaa

SEQ ID NO: 604 - DNA, 37 bp, Homo sapiens
  source 1..37: mol_type = genomic DNA
gtctgttggtgcacaaaaagacacttatactatgaaa

SEQ ID NO: 605 - DNA, 57 bp, Homo sapiens
  source 1..57: mol_type = genomic DNA
gacttcttgggcatccctggatcccaggttaagaacttctgcactagagatacatga

SEQ ID NO: 606 - DNA, 28 bp, Homo sapiens
  source 1..28: mol_type = genomic DNA
gtacagtatactgatctttctgggatag

SEQ ID NO: 607 - DNA, 34 bp, Homo sapiens
  source 1..34: mol_type = genomic DNA
gggctcaagggatctgcttacctcggcctcctaa

SEQ ID NO: 608 - DNA, 27 bp, Homo sapiens
  source 1..27: mol_type = genomic DNA
tatgactaaacgattatatgatgagaa

SEQ ID NO: 609 - DNA, 36 bp, Homo sapiens
  source 1..36: mol_type = genomic DNA
cttctaggagatttgtttggcgtgccaagcttctct

SEQ ID NO: 610 - DNA, 30 bp, Homo sapiens
  source 1..30: mol_type = genomic DNA
attgtaaaaagccatctgggctaacatttc

SEQ ID NO: 611 - DNA, 96 bp, Homo sapiens
  source 1..96: mol_type = genomic DNA
aggttaaattgcataagggtttgtgttagactgatagcatatctactgagtagcgccccgccgccccccgcccaccaccaagtttctgatcctttt

SEQ ID NO: 612 - DNA, 27 bp, Homo sapiens
  source 1..27: mol_type = genomic DNA
aatcatcggactcaggtacatctgtga

SEQ ID NO: 613 - DNA, 31 bp, Homo sapiens
  source 1..31: mol_type = genomic DNA
gtgagaacaggtgtcaccttgaaggtgggag

SEQ ID NO: 614 - DNA, 57 bp, Homo sapiens
  source 1..57: mol_type = genomic DNA
tcacatttggtttctagaccatctacctcatctagaaggagagcaattagtgagaca

SEQ ID NO: 615 - DNA, 72 bp, Homo sapiens
  source 1..72: mol_type = genomic DNA
atatttatttgacgcattcacacagctttttgatattctttctctaatgaaattagtgcttttagacttaat

SEQ ID NO: 616 - DNA, 51 bp, Homo sapiens
  source 1..51: mol_type = genomic DNA
tcagatgaattatctggtgaacgacaaagaaaacgccacaaatctgatagt

SEQ ID NO: 617 - DNA, 74 bp, Homo sapiens
  source 1..74: mol_type = genomic DNA
aaagcctggctctgtgtgtaataagggagatatgttgtgaaagaagcagtagcagtgaatctacagggacgcca

SEQ ID NO: 618 - DNA, 75 bp, Homo sapiens
  source 1..75: mol_type = genomic DNA
ttcagtttcagatcagtttagtgtagaatttgaagttgaatctctcgactcagaagattatagccttagtgaaga

SEQ ID NO: 619 - DNA, 77 bp, Homo sapiens
  source 1..77: mol_type = genomic DNA
tgcaatgaaatgaatcccccccttccatcacattgcaacagatgttgggcccttcgtgagaattggcttcctgaaga

SEQ ID NO: 620 - DNA, 69 bp, Homo sapiens
  source 1..69: mol_type = genomic DNA
ctatagtgaatgattccagagagtcatgtgttgaggaaaatgatgataaaattacacaagcttcacaat

SEQ ID NO: 621 - DNA, 25 bp, Homo sapiens
  source 1..25: mol_type = genomic DNA
tctcagccatcaacttctagtagca

SEQ ID NO: 622 - DNA, 41 bp, Homo sapiens
  source 1..41: mol_type = genomic DNA
aagagagtgtggaatctagtttgccccttaatgccattgaa

SEQ ID NO: 623 - DNA, 32 bp, Homo sapiens
  source 1..32: mol_type = genomic DNA
tcaaggtcgacctaaaaatggttgcattgtcc

SEQ ID NO: 624 - DNA, 26 bp, Homo sapiens
  source 1..26: mol_type = genomic DNA
atatttctaactatataaccctagga

SEQ ID NO: 625 - DNA, 722 bp, Homo sapiens
  source 1..722: mol_type = genomic DNA
accgcgtccggcctaaatgtcacttagtacctttgatataaagagaaaatgtgtgaaagatttagttttttgtttttttgtttgtttgtttgtttgtttgttttgagatgagtctctctgtcgcccaggctggagtgcagtgtcatgatctagcagtctccgcttcccgggttcaagccattctcctggctcagcctctggagcagctgggattacaggcatgcaccaccatgcccagctaatttttgtatttttagtagagatagggtttcaccatgttggccaggctggtcacgaactcctgacctcaagtgaggtcacccgcctcggcctcccgaagtgctgggattgcagatgtgagccaccatgtccagccaagaattagtatttaaattttagatactctttttttttttttttttttttttttttgagacagagtcttgctccatcacccatgctagagtgcagtggagtgatctcggctcactgcaacttccgccttctgggttcaagctattctcctgcctcagccttccaagtaactgggattacaggcatgtaccaccataccagctgatttttttgtatttttagtaaagacagggtttcaccatgttagccaggctgatcttgaactcctaaactcaagtgatctactcacctcagcctcccaaaatgctgggattacagatgtgaggcacctggcctcagatttttga

SEQ ID NO: 626 - DNA, 27 bp, Homo sapiens
  source 1..27: mol_type = genomic DNA
tggaggcccatccgagctcagcactga

SEQ ID NO: 627 - DNA, 371 bp, Homo sapiens
  source 1..371: mol_type = genomic DNA
acaagcctgtcaaatatctgcaagaactatggaataaaactactgatgcagtgaagacagttgaaaagatcaaacaaatgccaagctatatttataatgaacaaattcaagaaaaaggactacggaaagttcaggacatcaaagaagtcaggcaaaactcatcttgacccctgttgcaggcaaaggaacgcagctggaagaaaagatgatataacagttaacaggatgcagacatggcagaggtttcctaaaaatctcattatctataaccatttctatatttacatttgaaaatctcctttggagacttagaacctctaaattattgacttattttttatataaggtcactccgatgaaaggtgattaca

SEQ ID NO: 628 - DNA, 153 bp, Homo sapiens
  source 1..153: mol_type = genomic DNA
tgagagccgaataaggtttgcctgaaataactgacactatataatttctgctttggcaaatactaagttctaacttgtcattcctggtagaacaagctttatttttcgagcctagcaatgatctagaagcagatgttatctcagtgccttttg

SEQ ID NO: 629 - DNA, 226 bp, Homo sapiens
  source 1..226: mol_type = genomic DNA
ctgatgggtgtgctaattacactgatttgatcaatacccattgtatgtgaaacagtacatacaccatatttacaattatgtatttaacatttaaaatttctaatataagtatctctcaaactgtggattaacttcttgatttatatttaaatatgaatcttaagcaaaacagtgaaaataaccatcttgatttagtgtttttctcccatatgtgaattgtatatac

SEQ ID NO: 630 - DNA, 49 bp, Homo sapiens
  source 1..49: mol_type = genomic DNA
gcttagaataggactgaggtaattctgcacagcaactttactaatggta

SEQ ID NO: 631 - DNA, 68 bp, Homo sapiens
  source 1..68: mol_type = genomic DNA
gtcctccaagcattatttggagttgataatacttcagctacaaccaagcagaatctcttttttttgga

SEQ ID NO: 632 - DNA, 59 bp, Homo sapiens
  source 1..59: mol_type = genomic DNA
gataatacttcagcttcaatttggagttgataatatttcagctagaacctagtagaatc

SEQ ID NO: 633 - DNA, 123 bp, Homo sapiens
  source 1..123: mol_type = genomic DNA
cacttgatatatggaggcagtgacagctatttttacaaaatttaaatctgcaaatggattcaacatgtttatgggttattaaaattgtctgatttcttaggttctttatagtacacgtgttga

SEQ ID NO: 634 - DNA, 524 bp, Homo sapiens
  source 1..524: mol_type = genomic DNA
tgggagcctccaatgagagcaacttgagagaatgatgttgcaagttagtaggagtaagaaatgctgtgttctccctgtcttctcttaggtcacatggcagcctggcctaagtgatcgtgaatggtctataagggaggtagctgggacagggaggggagtttgggctagccaccgtaccacttgtcagcgtgaaaagtaagattgtaattgcctgtttagttttctgcctcatctttgaaagttccaccaagctgggaacctcttgattgtgaggcacaaatgtaagtacatcagaaaaaaacaaaaaaactggctttaaagcaggagcttgtgggcccctaagccagacggggactagcttttggcattatataattaagattttttaaatccttaataagggttttattttatttttatttattttttgagacggagtcttgctctgtggctcaggctggagtacagtggtgcaatcttggctcactgcaacctctgcctcctggctgtgttcaagtggttct

SEQ ID NO: 635 - DNA, 156 bp, Homo sapiens
  source 1..156: mol_type = genomic DNA
gaaacattaagaataccatatgagtaaattaaacactttggctcttttcggaaaaaaacagatgagctcttatattttaaagtttggttttgacagaaaaaaattcctagattttttggttaataaaaactttattagatagattaaattgtgatg

SEQ ID NO: 636 - DNA, 279 bp, Homo sapiens
  source 1..279: mol_type = genomic DNA
agggaacttctgcttaagaggcttctatgtaatgaaattctcttgaaaacagagaaactatttcctgtttattttctaaattgagacgtcactttttaaaaattggtacctgtaatttagccatttcctactcagcaatgtctcatttaaactattatttgtttagcgtgtttcaaagagcagatgtaagcttgagcccatcctctgtcctatgactaagtcgatattagcaggggttaggactgttagttttccagttcctactggaggcaaattctt

SEQ ID NO: 637 - DNA, 172 bp, Homo sapiens
  source 1..172: mol_type = genomic DNA
ccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagtt

SEQ ID NO: 638 - DNA, 171 bp, Homo sapiens
  source 1..171: mol_type = genomic DNA
tcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccac

SEQ ID NO: 639 - DNA, 35 bp, Homo sapiens
  source 1..35: mol_type = genomic DNA
actcagtggagaaggagttggaccagtcaacatct

SEQ ID NO: 640 - DNA, 172 bp, Homo sapiens
  source 1..172: mol_type = genomic DNA
aggtcaatccctcttcattggaaaatccctctggagagttctcccttcctttaacttaagcagcttttgggtgtacagactcctggcttatggaatgaactcgaatcatgaggatgggagttagccacatagactaatgctgtctttttgggagctgttaacccttaattca

SEQ ID NO: 641 - DNA, 290 bp, Homo sapiens
  source 1..290: mol_type = genomic DNA
caagatggccttcagctgataaagcgaagctgctctactgtggggtgtacaacacacatacatgagatcagtgacttgtgcgtgataatgacacatcatcaacactatttcagtctgactcatggccatatagctgacctcaactcacttttctggtctcttttcccccaccggtgttcctgggcactggctgtcctccaagcacctgagcaactcagcaatcttcttgacacttgtgccttttctgcttttgctcacgtcctttgctcaacctcaatatccatgtcatg

SEQ ID NO: 642 - DNA, 37 bp, Homo sapiens
  source 1..37: mol_type = genomic DNA
tgcgccaatctgcagaacgccattgcggatgccgagc

SEQ ID NO: 643 - DNA, 119 bp, Homo sapiens
  source 1..119: mol_type = genomic DNA
cttttgctaaacacacgcagctagatccagtaccagtgtttcagtgtcctgccacccacgatgtactggtttctctctgggattcatgatagtttggtttgtctgacccagaaactcag

SEQ ID NO: 644 - DNA, 101 bp, Homo sapiens
  source 1..101: mol_type = genomic DNA
agctggccggcatggcgatgacctccgcaacaccaagcatgagatctctgagatgaaccggatgatccagaggctgagagccgagattgacaatgtcaaga

SEQ ID NO: 645 - DNA, 156 bp, Homo sapiens
  source 1..156: mol_type = genomic DNA
cagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaag

SEQ ID NO: 646 - DNA, 32 bp, Homo sapiens
  source 1..32: mol_type = genomic DNA
cagaaccagatgaccgactccaaatctccctg

SEQ ID NO: 647 - DNA, 35 bp, Homo sapiens
  source 1..35: mol_type = genomic DNA
ggccaaggttgatgcactgatggatgagattaact

SEQ ID NO: 648 - DNA, 57 bp, Homo sapiens
  source 1..57: mol_type = genomic DNA
gcagcctgcagctatgctctctaagcgtggagctcacttgagtagggtgacggtgtg

SEQ ID NO: 649 - DNA, 34 bp, Homo sapiens
  source 1..34: mol_type = genomic DNA
tgaaatcaacaagcgtaccactgctgagaatgag

SEQ ID NO: 650 - DNA, 26 bp, Homo sapiens
  source 1..26: mol_type = genomic DNA
aaaagaacactagagaaattgactag

SEQ ID NO: 651 - DNA, 36 bp, Homo sapiens
  source 1..36: mol_type = genomic DNA
gtggacacgttctgaattagactggcagctgggaag

SEQ ID NO: 652 - DNA, 62 bp, Homo sapiens
  source 1..62: mol_type = genomic DNA
caacctgcaaatcgaccccagcatccagagggtgaggaccgaggagcgcgagcagatcaaga

SEQ ID NO: 653 - DNA, 47 bp, Homo sapiens
  source 1..47: mol_type = genomic DNA
gaggtggtgccggtagtggatttggtttcggcggtggagctggtggt

SEQ ID NO: 654 - DNA, 32 bp, Homo sapiens
  source 1..32: mol_type = genomic DNA
ggcagcttcaggaaccggtttggtgctggtgc

SEQ ID NO: 655 - DNA, 32 bp, Homo sapiens
  source 1..32: mol_type = genomic DNA
gggctccaagaggatatccatcagcactagtg

SEQ ID NO: 656 - DNA, 44 bp, Homo sapiens
  source 1..44: mol_type = genomic DNA
tgtggagtgggtggctatggcagccggagcctctacaacctggg

SEQ ID NO: 657 - DNA, 29 bp, Homo sapiens
  source 1..29: mol_type = genomic DNA
tggtggcttcggcagggtcagccttgcgg

SEQ ID NO: 658 - DNA, 80 bp, Homo sapiens
  source 1..80: mol_type = genomic DNA
tcaagtgtgtccttccggagcgggggcagtcgtagcttcagcaccgcctctgccatcaccccgtctgtctcccgcaccag

SEQ ID NO: 659 - DNA, 137 bp, Homo sapiens
  source 1..137: mol_type = genomic DNA
acagctcgacagctctctcgcccagcccagttctggaagggataaaaagggggcatcaccgttcctgggtaacagagccaccttctgcgtcctgctgagctctgttctctccagcacctcccaacccactagtgcct

SEQ ID NO: 660 - DNA, 102 bp, Homo sapiens
  source 1..102: mol_type = genomic DNA
cccagcctctatggtgaagacatacttgctagcagcgtcaccaacttgctgccaagagatcagtgctgcaaggcaaggttatttctaactgagcagagcctg

SEQ ID NO: 661 - DNA, 655 bp, Homo sapiens
  source 1..655: mol_type = genomic DNA
caggtctgtggcaatactcttaaccataagaattgaaatggtgaagaaacaagtatacactagaggctcttaaaagtattgaaagacaatactgctgttatatagcaagacataaacagattataaacatcagagccatttgcttctcagtttacatttctgatacatgcagatagcagatgtctttaaatgaaatacatgtatattgtgtatggacttaattatgcacatgctcagatgtgtagacatcctccgtatatttacataacatatagaggtaatagataggtgatatacatgatacattctcaagagttgcttgaccgaaagttacaaggaccccaacccctttgtcctctctacccacagatggccctgggaatcaattcctcaggaattgccctcaagaactctgcttcttgctttgcagagtgccatggtcatgtcattctgaggtcacataacacataaaattagtttctatgagtgtataccatttaaagaatttttttttcagtaaaagggaatattacaatgttggaggagagataagttatagggagctggatttcaaaacgtggtccaagattcaaaaatcctattgatagtggccattttaatcattgccatcgtgtgcttgtttcatccagtgtta

SEQ ID NO: 662 - DNA, 35 bp, Homo sapiens
  source 1..35: mol_type = genomic DNA
gtgtgtattccagacccgtcctaaacacttcctag

SEQ ID NO: 663 - DNA, 118 bp, Homo sapiens
  source 1..118: mol_type = genomic DNA
tcggagcagcagcataagctggacttcaaggcatacgaacaggcactgcaatactcgccttacggctctacgttgcccgccagcctgcctctaggcagcgcctcggtgaccaccagga

SEQ ID NO: 664 - DNA, 50 bp, Homo sapiens
  source 1..50: mol_type = genomic DNA
gggcgcctcggagttgaagactccagcctcctcaactgcgccccccataa

SEQ ID NO: 665 - DNA, 96 bp, Homo sapiens
  source 1..96: mol_type = genomic DNA
ggcggccctgagagccgcaaggacccctctggcgcctctaaccccagcgccgactcgcccctccatcggggtgtgcacgggaagaccggccagcta

SEQ ID NO: 666 - DNA, 206 bp, Homo sapiens
  source 1..206: mol_type = genomic DNA
aggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatg

SEQ ID NO: 667 - DNA, 68 bp, Homo sapiens
  source 1..68: mol_type = genomic DNA
tacgcgccgtccaacctgggccgcagccgcgcgggcggcggcggcgacgccaagacgttcaagcgcag

SEQ ID NO: 668 - DNA, 210 bp, Homo sapiens
  source 1..210: mol_type = genomic DNA
tccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgact

SEQ ID NO: 669 - DNA, 53 bp, Homo sapiens
  source 1..53: mol_type = genomic DNA
tggaagggcatgaaaccagcgactggaacagctactacgcagacacgcaggag

SEQ ID NO: 670 - DNA, 27 bp, Homo sapiens
  source 1..27: mol_type = genomic DNA
ctttgtgcggcggacaaatggggagag

SEQ ID NO: 671 - DNA, 48 bp, Homo sapiens
  source 1..48: mol_type = genomic DNA
tcataaagatataaaccggtgctgtgactcacctgctcttagccgcag

SEQ ID NO: 672 - DNA, 63 bp, Homo sapiens
  source 1..63: mol_type = genomic DNA
ggggtcggagctgatcgggcgcctagccccgcgcctgggcctcgccgagcccgacatgctgag

SEQ ID NO: 673 - DNA, 53 bp, Homo sapiens
  source 1..53: mol_type = genomic DNA
gtgagttcggccgcgcaagaccagggctgggcttccgcctcgcggccctgggc

SEQ ID NO: 674 - DNA, 89 bp, Homo sapiens
  source 1..89: mol_type = genomic DNA
ctctcaccagaggatatgaccttcattcccagccccagataaacgagccacaggagttaggcttagtgtgaagctaaccaggctgtatt

SEQ ID NO: 675 - DNA, 75 bp, Homo sapiens
  source 1..75: mol_type = genomic DNA
aagcagaggagtacttgcgcctgtcccgggtgaagtgtgtcggcctctccgcacgcaccacggagaccagcagtg

SEQ ID NO: 676 - DNA, 29 bp, Homo sapiens
  source 1..29: mol_type = genomic DNA
gatcttgacttatccaggccacttttcac

SEQ ID NO: 677 - DNA, 143 bp, Homo sapiens
  source 1..143: mol_type = genomic DNA
gccaagtgactatattcccagtttatcccataatgtagctaacaacttggaactagtgttgccagaattccactagcaaatagcagctgtatatatatgctgggaattctgatttcagtctgccttttgtaagagatgatatc

SEQ ID NO: 678 - DNA, 234 bp, Homo sapiens
  source 1..234: mol_type = genomic DNA
aagtcatgaagcactacagcaaatgtcttttatgtgccccttttgttataaaatagatcccatgtgcattttaactctcagtccaataaacaactaaacaacttagcatagataataacatgtttggaatgaaggaaaaaaactagacagaggctctggaagcatggtcaaaaagaaaataagttgattatctggttgcccagagaagaaaactgtacaggtcttgagaaaagc

SEQ ID NO: 679 - DNA, 30 bp, Homo sapiens
  source 1..30: mol_type = genomic DNA
gattctaaagctgaaagtggataaaaacaa

SEQ ID NO: 680 - DNA, 60 bp, Homo sapiens
  source 1..60: mol_type = genomic DNA
aaaaagctatatttgatcgactgtgtaaacaactagagaagattggacagcaggtcgaca

SEQ ID NO: 681 - DNA, 40 bp, Homo sapiens
  source 1..40: mol_type = genomic DNA
ttatttgtgatccaaaacatgcccaaatactgaaattgag

SEQ ID NO: 682 - DNA, 56 bp, Homo sapiens
  source 1..56: mol_type = genomic DNA
acctggagatgtagctactccaccacggaagagaaagaagatagtggttgaagccc

SEQ ID NO: 683 - DNA, 50 bp, Homo sapiens
  source 1..50: mol_type = genomic DNA
aaggtagaggagatgccacataaaccacagaaagatgaagatctgacaca

SEQ ID NO: 684 - DNA, 41 bp, Homo sapiens
  source 1..41: mol_type = genomic DNA
ttggaaaatgctgccagtgctcaaaaggctacagcagagtg

SEQ ID NO: 685 - DNA, 510 bp, Homo sapiens
  source 1..510: mol_type = genomic DNA
tctccaggaagacttgacggctttgggattttgtttaaacttttataataaggatcctaagactgttgcctttaaatagcaaagcagcctacctggaggctaagtctgggcagtgggctggcccctggtgtgagcattagaccagccacagtgcctgattggtatagccttatgtgctttcctacaaaatggaattggaggccgggcgcagtggctcacgcctgtaatcccagcactttgggaggccaaggtgggtggatcacctgaggtcaggagctcgagaccagcctggccaacatggtgaaaccccatctctactaaaaatacaaaaattagccaggtgtgatggtgcatgcctgtaatcccagctcctcagtaggctgagacaggagcatcacttgaacgtgggaggcagaggttgcagtgagccgagattgcaccaccgcactccagcctgggtgacagagcgagacttatctcataaataaatagatagatactccagcctgg

SEQ ID NO: 686 - DNA, 153 bp, Homo sapiens
  source 1..153: mol_type = genomic DNA
tggagccattttgctttaagtgaatggcagtcccttgtcttattcagaatataaaattcagtctgaatggcatcttacagattttacttcaatttttgtgtacggtattttttatttgactaaatcaatatattgtacagcctaagttaataa

SEQ ID NO: 687 - DNA, 165 bp, Homo sapiens
  source 1..165: mol_type = genomic DNA
cggagcctccgttttcagtcgacttcagatgtgtctccacttttttccgctgtagccgcaaggcaaggaaacatttctcttcccgtactgaggaggctgaggagtgcactgggtgttcttttctcctctaacccagaactgcgagacagaggctgagtccctgta

SEQ ID NO: 688 - DNA, 36 bp, Homo sapiens
  source 1..36: mol_type = genomic DNA
atggggctccctcgtggacctctcgcgtctctcctc

SEQ ID NO: 689 - DNA, 70 bp, Homo sapiens
  source 1..70: mol_type = genomic DNA
gctggctgcagtgcgcggcctccgagccgtgccgggcggtcttcagggaggctgaagtgaccttggaggc

SEQ ID NO: 690 - DNA, 63 bp, Homo sapiens
  source 1..63: mol_type = genomic DNA
aagagccagctctgtttagcactgataatgatgacttcactgtgcggaatggcgagacagtcc

SEQ ID NO: 691 - DNA, 35 bp, Homo sapiens
  source 1..35: mol_type = genomic DNA
gatcttcccatccaaacgtatcttacgaagacaca

SEQ ID NO: 692 - DNA, 25 bp, Homo sapiens
  source 1..25: mol_type = genomic DNA
tgggtggttgctccaatatctgtcc

SEQ ID NO: 693 - DNA, 108 bp, Homo sapiens
  source 1..108: mol_type = genomic DNA
tttctacagcatcacggggccgggggcagacagcccccctgagggtgtcttcgctgtagagaaggagacaggctggttgttgttgaataagccactggaccgggagga

SEQ ID NO: 694 - DNA, 112 bp, Homo sapiens
  source 1..112: mol_type = genomic DNA
ctcagtggaggaccccatgaacatctccatcatcgtgaccgaccagaatgaccacaagcccaagtttacccaggacaccttccgagggagtgtcttagagggagtcctacca

SEQ ID NO: 695 - DNA, 130 bp, Homo sapiens
  source 1..130: mol_type = genomic DNA
tgacagccacggatgaggatgatgccatctacacctacaatggggtggttgcttactccatccatagccaagaaccaaaggacccacacgacctcatgttcaccattcaccggagcacaggcaccatcag

SEQ ID NO: 696 - DNA, 137 bp, Homo sapiens
  source 1..137: mol_type = genomic DNA
aggcccatgtgcctgagaatgcagtgggccatgaggtgcagaggctgacggtcactgatctggacgcccccaactcaccagcgtggcgtgccacctaccttatcatgggcggtgacgacggggaccattttaccatc

SEQ ID NO: 697 - DNA, 52 bp, Homo sapiens
  source 1..52: mol_type = genomic DNA
ccagcacaccctgtacgttgaagtgaccaacgaggccccttttgtgctgaag

SEQ ID NO: 698 - DNA, 83 bp, Homo sapiens
  source 1..83: mol_type = genomic DNA
acagccaccatagtggtccacgtggaggatgtgaatgaggcacctgtgtttgtcccaccctccaaagtcgttgaggtccagga

SEQ ID NO: 699 - DNA, 27 bp, Homo sapiens
  source 1..27: mol_type = genomic DNA
gctgtgggcaccctcgaccgtgaggat

SEQ ID NO: 700 - DNA, 188 bp, Homo sapiens
  source 1..188: mol_type = genomic DNA
acgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggc

SEQ ID NO: 701 - DNA, 105 bp, Homo sapiens
  source 1..105: mol_type = genomic DNA
gtggtcttgtccctgaagaagttcctgaagcaggatacatatgacgtgcacctttctctgtctgaccatggcaacaaagagcagctgacggtgatcagggccact

SEQ ID NO: 702 - DNA, 38 bp, Homo sapiens
  source 1..38: mol_type = genomic DNA
gtgtgcgactgccatggccatgtcgaaacctgccctgg

SEQ ID NO: 703 - DNA, 51 bp, Homo sapiens
  source 1..51: mol_type = genomic DNA
ctcccagaagatgacacccgtgacaacgtcttctactatggcgaagagggg

SEQ ID NO: 704 - DNA, 118 bp, Homo sapiens
  source 1..118: mol_type = genomic DNA
cacccagctccaccgaggtctggaggccaggccggaggtggttctccgcaatgacgtggcaccaaccatcatcccgacacccatgtaccgtcctcggccagccaacccagatgaaatc

SEQ ID NO: 705 - DNA, 41 bp, Homo sapiens
  source 1..41: mol_type = genomic DNA
gcaagaggcaggacccgccgctcctaactacctgttctctg

SEQ ID NO: 706 - DNA, 158 bp, Homo sapiens
  source 1..158: mol_type = genomic DNA
acctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggc

SEQ ID NO: 707 - DNA, 385 bp, Homo sapiens
  source 1..385: mol_type = genomic DNA
tctgacgttagagtggtggcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatac

SEQ ID NO: 708 - DNA, 129 bp, Homo sapiens
  source 1..129: mol_type = genomic DNA
agagaacctacccaagatgtcagtgaaattggaacattcctgacaataccagggcataaatgcaggaatcaggaataggcagcagtgatagaacaattctgtttgtgcccttgttaacgtgaagttcaa

SEQ ID NO: 709 - DNA, 133 bp, Homo sapiens
  source 1..133: mol_type = genomic DNA
ttcccggatttttgtgggcgcctgccccgcccctcgtccccctgctgtgtccatatatcgaggcgatagggttaagggaaggcggacgcctgatgggttaatgagcaaactgaagtgttttccatgatctttt

SEQ ID NO: 710 - DNA, 60 bp, Homo sapiens
  source 1..60: mol_type = genomic DNA
cgcaattgaagtaccacctcccgagggtgattgcttccccatgcggggtagaacctttgc

SEQ ID NO: 711 - DNA, 194 bp, Homo sapiens
  source 1..194: mol_type = genomic DNA
ccagtggtctatacctccagcagcaagtcgagtgagcaagtgatgtcctgaaaggcccagtggatcagtggaatgaagcgggcaggaagacttagtgctcctgaaacaaggaatccagaatccaggagaaggatggctcagtggggctttcaagggacaagtatgggggttgaaggggtcactgtccctatacc

SEQ ID NO: 712 - DNA, 109 bp, Homo sapiens
  source 1..109: mol_type = genomic DNA
ggcaaagcaaagctatattcaagaccacatgcaaagctactccctgagcaaagagtcacagataaaacgggggcaccagtagaatggccaggacaaacgcagtgcagca

SEQ ID NO: 713 - DNA, 32 bp, Homo sapiens
  source 1..32: mol_type = genomic DNA
gatgagagtgacatgtactgttgtggacatgc

SEQ ID NO: 714 - DNA, 25 bp, Homo sapiens
  source 1..25: mol_type = genomic DNA
tgccccgggggtcctggaagccaca

SEQ ID NO: 715 - DNA, 108 bp, Homo sapiens
  source 1..108: mol_type = genomic DNA
agaatgaagttgtgaagctgagattcccctccattgggaccggagaaaccaggggagccccccgggcagccgcgcgccccttcccacggggccctttactgcgccgcg

SEQ ID NO: 716 - DNA, 26 bp, Homo sapiens
  source 1..26: mol_type = genomic DNA
cagccggagccatggggccggagccg

SEQ ID NO: 717 - DNA, 37 bp, Homo sapiens
  source 1..37: mol_type = genomic DNA
gagctggcggccttgtgccgctgggggctcctcctcg

SEQ ID NO: 718 - DNA, 141 bp, Homo sapiens
  source 1..141: mol_type = genomic DNA
tgtgcaccggcacagacatgaagctgcggctccctgccagtcccgagacccacctggacatgctccgccacctctaccagggctgccaggtggtgcagggaaacctggaactcacctacctgcccaccaatgccagcctgt

SEQ ID NO: 719 - DNA, 149 bp, Homo sapiens
  source 1..149: mol_type = genomic DNA
ttgggagcagttgtgaagctcagaagagaaatgtctgtgaaaaggttatgaacaggagggagagtggaaaccaacctgctggatcgtgtccacagaccctggaatggggccacatgcttggtttgtcaaattgcagacgccggccgggt

SEQ ID NO: 720 - DNA, 133 bp, Homo sapiens
  source 1..133: mol_type = genomic DNA
cagggctacgtgctcatcgctcacaaccaagtgaggcaggtcccactgcagaggctgcggattgtgcgaggcacccagctctttgaggacaactatgccctggccgtgctagacaatggagacccgctgaaca

SEQ ID NO: 721 - DNA, 32 bp, Homo sapiens
  source 1..32: mol_type = genomic DNA
aggggaaagggtcctctgatcattgctcaccc

SEQ ID NO: 722 - DNA, 118 bp, Homo sapiens
  source 1..118: mol_type = genomic DNA
ggtcttgatccagcggaacccccagctctgctaccaggacacgattttgtggaaggacatcttccacaagaacaaccagctggctctcacactgatagacaccaaccgctctcgggcc

SEQ ID NO: 723 - DNA, 52 bp, Homo sapiens
  source 1..52: mol_type = genomic DNA
gccactgcccactgactgctgccatgagcagtgtgctgccggctgcacgggc

SEQ ID NO: 724 - DNA, 74 bp, Homo sapiens
  source 1..74: mol_type = genomic DNA
tggtcacctacaacacagacacgtttgagtccatgcccaatcccgagggccggtatacattcggcgccagctgt

SEQ ID NO: 725 - DNA, 107 bp, Homo sapiens
  source 1..107: mol_type = genomic DNA
acctttctacggacgtgggatcctgcaccctcgtctgccccctgcacaaccaagaggtgacagcagaggatggaacacagcggtgtgagaagtgcagcaagccctgt

SEQ ID NO: 726 - DNA, 58 bp, Homo sapiens
  source 1..58: mol_type = genomic DNA
tgcagttcctgtccctctgcgcatgcagcctggcccagcccaccctgtcctatccttc

SEQ ID NO: 727 - DNA, 63 bp, Homo sapiens
  source 1..63: mol_type = genomic DNA
tacaagtgtccctatatcccctgtcagtgtggggaggggcccggaccctgatgctcatgtggc

SEQ ID NO: 728 - DNA, 82 bp, Homo sapiens
  source 1..82: mol_type = genomic DNA
ggcatggagcacttgcgagaggtgagggcagttaccagtgccaatatccaggagtttgctggctgcaagaagatctttggga

SEQ ID NO: 729 - DNA, 57 bp, Homo sapiens
  source 1..57: mol_type = genomic DNA
aacactgccccgctccagccagagcagctccaagtgtttgagactctggaagagatc

SEQ ID NO: 730 - DNA, 155 bp, Homo sapiens
  source 1..155: mol_type = genomic DNA
ggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgca

SEQ ID NO: 731 - DNA, 246 bp, Homo sapiens
  source 1..246: mol_type = genomic DNA
agcagcgttcttggacttgtgcagactgcccgtctctgtgcacccttcttgactcagcacagctctggctggcttggcctcttggcatggcttctctagctgggtcctacctgccttggcatccttccctccccctctgtttctgaaatctcagaactcttcctctccctacatcggccccacctgtccccacccctccagcccacagccatgcccacagccagttccctggttcacttggacctg

SEQ ID NO: 732 - DNA, 104 bp, Homo sapiens
  source 1..104: mol_type = genomic DNA
gccaccagctgtgcgcccgagggcactgctggggtccagggcccacccagtgtgtcaactgcagccagttccttcggggccaggagtgcgtggaggaatgccga

SEQ ID NO: 733 - DNA, 47 bp, Homo sapiens
  source 1..47: mol_type = genomic DNA
tggctggaggggtgcatggggctcctctcagaccccctcaccactgt

SEQ ID NO: 734 - DNA, 32 bp, Homo sapiens
  source 1..32: mol_type = genomic DNA
gctccccagggagtatgtgaatgccaggcact

SEQ ID NO: 735 - DNA, 223 bp, Homo sapiens
  source 1..223: mol_type = genomic DNA
actcgctgttacaccttaggtaatgcgttttcctctctgggtgcctcccattttctggctcaagtccctgcccaggatcaagcttggaggagggccccgagggaggggccacagagactgggtgaagagcaagggtgtttgtcccaggagcatggcgaaaattgctgctgggtggccttgggaagcacaaaggggacccaactaagggcctgatcctactgcc

SEQ ID NO: 736 - DNA, 119 bp, Homo sapiens
  source 1..119: mol_type = genomic DNA
gtggcctgtgcccactataaggaccctcccttctgcgtggcccgctgccccagcggtgtgaaacctgacctctcctacatgcccatctggaagtttccagatgaggagggcgcatgcca

SEQ ID NO: 737 - DNA, 75 bp, Homo sapiens
  source 1..75: mol_type = genomic DNA
gtgagtccaacggtcttttctgcagaaaggaggactttcctttcaggggtctttctggggctcttactataaaag

SEQ ID NO: 738 - DNA, 25 bp, Homo sapiens
  source 1..25: mol_type = genomic DNA
gggctgccccgccgagcagagagcc

SEQ ID NO: 739 - DNA, 164 bp, Homo sapiens
  source 1..164: mol_type = genomic DNA
ttccactgtggaacctcctgtcattttccacttcaccaagtgacagaggacctgctcagatgctgaggggaggggactgcaaggaaagatggctaggaaacccagtccctccacaccctagagtaacttgatgccttgtgagggacacaggcaaagttcaattc

SEQ ID NO: 740 - DNA, 73 bp, Homo sapiens
  source 1..73: mol_type = genomic DNA
gtggtctttgggatcctcatcaagcgacggcagcagaagatccggaagtacacgatgcggagactgctgcagg

SEQ ID NO: 741 - DNA, 81 bp, Homo sapiens
  source 1..81: mol_type = genomic DNA
agcgatgcccaaccaggcgcagatgcggatcctgaaagagacggagctgaggaaggtgaaggtgcttggatctggcgcttt

SEQ ID NO: 742 - DNA, 96 bp, Homo sapiens
  source 1..96: mol_type = genomic DNA
gcatctggatccctgatggggagaatgtgaaaattccagtggccatcaaagtgttgagggaaaacacatcccccaaagccaacaaagaaatcttag

SEQ ID NO: 743 - DNA, 135 bp, Homo sapiens
  source 1..135: mol_type = genomic DNA
tgggcatctgcctgacatccacggtgcagctggtgacacagcttatgccctatggctgcctcttagaccatgtccgggaaaaccgcggacgcctgggctcccaggacctgctgaactggtgtatgcagattgcca

SEQ ID NO: 744 - DNA, 118 bp, Homo sapiens
  source 1..118: mol_type = genomic DNA
atgtgcggctcgtacacagggacttggccgctcggaacgtgctggtcaagagtcccaaccatgtcaaaattacagacttcgggctggctcggctgctggacattgacgagacagagta

SEQ ID NO: 745 - DNA, 37 bp, Homo sapiens
  source 1..37: mol_type = genomic DNA
ctgggtggagtggtgtctagcccatgggagaactctg

SEQ ID NO: 746 - DNA, 41 bp, Homo sapiens
  source 1..41: mol_type = genomic DNA
ctggagtccattctccgccggcggttcacccaccagagtga

SEQ ID NO: 747 - DNA, 75 bp, Homo sapiens
  source 1..75: mol_type = genomic DNA
actgtgtgggagctgatgacttttggggccaaaccttacgatgggatcccagcccgggagatccctgacctgctg

SEQ ID NO: 748 - DNA, 31 bp, Homo sapiens
  source 1..31: mol_type = genomic DNA
gtgcgtggctgagctgtgctggctgcctgga

SEQ ID NO: 749 - DNA, 39 bp, Homo sapiens
  source 1..39: mol_type = genomic DNA
gttggatgattgactctgaatgtcggccaagattccggg

SEQ ID NO: 750 - DNA, 51 bp, Homo sapiens
  source 1..51: mol_type = genomic DNA
gttggtgtctgaattctcccgcatggccagggacccccagcgctttgtggt

SEQ ID NO: 751 - DNA, 44 bp, Homo sapiens
  source 1..44: mol_type = genomic DNA
tctaccgctcactgctggaggacgatgacatgggggacctggtg

SEQ ID NO: 752 - DNA, 228 bp, Homo sapiens
  source 1..228: mol_type = genomic DNA
gtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctga

SEQ ID NO: 753 - DNA, 32 bp, Homo sapiens
  source 1..32: mol_type = genomic DNA
atgtgaaccagccagatgttcggccccagccc

SEQ ID NO: 754 - DNA, 122 bp, Homo sapiens
  source 1..122: mol_type = genomic DNA
agccttcgacaacctctattactgggaccaggacccaccagagcggggggctccacccagcaccttcaaagggacacctacggcagagaacccagagtacctgggtctggacgtgccagtgt

SEQ ID NO: 755 - DNA, 25 bp, Homo sapiens
  source 1..25: mol_type = genomic DNA
agaaggccaagtccgcagaagccct

SEQ ID NO: 756 - DNA, 39 bp, Homo sapiens
  source 1..39: mol_type = genomic DNA
cgaccacttccaggggaacctgccatgccaggaacctgt

SEQ ID NO: 757 - DNA, 107 bp, Homo sapiens
  source 1..107: mol_type = genomic DNA
cttcctgcttgagttcccagatggctggaaggggtccagcctcgttggaagaggaacagcactggggagtctttgtggattctgaggccctgcccaatgagactcta

SEQ ID NO: 758 - DNA, 110 bp, Homo sapiens
  source 1..110: mol_type = genomic DNA
ggtactgaaagccttagggaagctggcctgagaggggaagcggccctaagggagtgtctaagaacaaaagcgacccattcagagactgtccctgaaacctagtactgccc

SEQ ID NO: 759 - DNA, 31 bp, Homo sapiens
  source 1..31: mol_type = genomic DNA
agtatccaggctttgtacagagtgcttttct

SEQ ID NO: 760 - DNA, 32 bp, Homo sapiens
  source 1..32: mol_type = genomic DNA
cctccctgaagacgtggtcccagccgggtgtc

SEQ ID NO: 761 - DNA, 57 bp, Homo sapiens
  source 1..57: mol_type = genomic DNA
ggagagggatcctctaaattgtcgaggcttcatctctccagattgtatgcccttctc

SEQ ID NO: 762 - DNA, 25 bp, Homo sapiens
  source 1..25: mol_type = genomic DNA
ctggttccgttaagcccctctcttg

SEQ ID NO: 763 - DNA, 84 bp, Homo sapiens
  source 1..84: mol_type = genomic DNA
atcttagcagctctccggaagacctttgcccagcccctgggacccctcctgggactccccggccccctgatacccctctgcctg

SEQ ID NO: 764 - DNA, 26 bp, Homo sapiens
  source 1..26: mol_type = genomic DNA
gtaaagaggtcccagcctctcctcat

SEQ ID NO: 765 - DNA, 38 bp, Homo sapiens
  source 1..38: mol_type = genomic DNA
gaaacttcgagaggaggagaggcgtgccacctccctcc

SEQ ID NO: 766 - DNA, 57 bp, Homo sapiens
  source 1..57: mol_type = genomic DNA
tctcgggggcccctccagtgcaagggggctgctcccccgcgatgccagccgccccca

SEQ ID NO: 767 - DNA, 89 bp, Homo sapiens
  source 1..89: mol_type = genomic DNA
tgggagatacacagccgcttccatggaggcaggggatcttggttaggagtccctgagggtctagcaggtgcggaaagggaatgaatcac

SEQ ID NO: 768 - DNA, 65 bp, Homo sapiens
  source 1..65: mol_type = genomic DNA
gccacagctcgccacgtgtgtgaaatgctggtgcagcgagctcacgccttgagcgacgagacctg

SEQ ID NO: 769 - DNA, 105 bp, Homo sapiens
  source 1..105: mol_type = genomic DNA
accacgagtccgtggtggaagtgcaggctgcctggcccgtgggcggagatagccgcttcgtcttccggaaaaacttcgccaagtacgaactgttcaagagctccc

SEQ ID NO: 770 - DNA, 73 bp, Homo sapiens
  source 1..73: mol_type = genomic DNA
tctgggcgctgggatgccctgatcctcaacctggatgctggagccctgatccctgacacttgtctacccacag

SEQ ID NO: 771 - DNA, 43 bp, Homo sapiens
  source 1..43: mol_type = genomic DNA
tccagctgtctcgatgcacacactggtatatcccatgaagacc

SEQ ID NO: 772 - DNA, 79 bp, Homo sapiens
  source 1..79: mol_type = genomic DNA
tcaggacggaagctttggaaacgctttttctgcttcttgcgccgatctggcctctattactccaccaagggcacctcta

SEQ ID NO: 773 - DNA, 132 bp, Homo sapiens
  source 1..132: mol_type = genomic DNA
cagtccctggtccttttagaagttgccccttctctgctggaacctctgagcccttctccccctgggccccccaggccagccacctccagtttaccatctctccctacatccttgcctagctcacctgcccag

SEQ ID NO: 774 - DNA, 107 bp, Homo sapiens
  source 1..107: mol_type = genomic DNA
atccgaggcacctgcagtacgtggcagatgtgaacgagtccaacgtgtacgtggtgacgcagggccgcaagctctacgggatgcccactgacttcggtttctgtgtc

SEQ ID NO: 775 - DNA, 76 bp, Homo sapiens
  source 1..76: mol_type = genomic DNA
caacaagcttcgaaatggccacaaggggcttcggatcttctgcagtgaagatgagcagagccgcacctgctggctg

SEQ ID NO: 776 - DNA, 77 bp, Homo sapiens
  source 1..77: mol_type = genomic DNA
tacggggtgcagctgtacaagaattaccagcaggcacagtctcgccatctgcatccatcttgtttgggctccccacc

SEQ ID NO: 777 - DNA, 83 bp, Homo sapiens
  source 1..83: mol_type = genomic DNA
agaagtgcctcagataataccctggtggccatggacttctctggccatgctgggcgtgtcattgagaacccccgggaggctct

SEQ ID NO: 778 - DNA, 78 bp, Homo sapiens
  source 1..78: mol_type = genomic DNA
ccatccaccgcacccaactctggttccacgggcgcatttcccgtgaggagagccagcggcttattggacagcagggct

SEQ ID NO: 779 - DNA, 72 bp, Homo sapiens
  source 1..72: mol_type = genomic DNA
ctgttcctggtccgggagagtcagcggaacccccagggctttgtcctctctttgtgccacctgcagaaagtg

SEQ ID NO: 780 - DNA, 128 bp, Homo sapiens
  source 1..128: mol_type = genomic DNA
aggagggccgcctgtacttcagcatggatgatggccagacccgcttcactgacctgctgcagctcgtggagttccaccagctgaaccgcggcatcctgccgtgcttgctgcgccattgctgcacgcgg

SEQ ID NO: 781 - DNA, 297 bp, Homo sapiens
  source 1..297: mol_type = genomic DNA
cccatccagtggactctggggcgcggccacaggggacgggatgaggagcgggagggttccgccactccagttttctcctctgcttctttgcctccctcagatagaaaacagcccccactccagtccactcctgacccctctcctcaagggaaggccttgggtggccccctctccttctcctagctctggaggtgctgctctagggcagggaattatgggagaagtgggggcagcccaggcggtttcacgccccacactttgtacagaccgagaggccagttgatctgctctgtttta

SEQ ID NO: 782 - DNA, 137 bp, Homo sapiens
  source 1..137: mol_type = genomic DNA
tggcctcacagcgactctaagacttggggctctctcattggctgtaactcttccactggattggtagcaaaaaaagaggcggtgcccaaggcgaaaggctctgtgactacagccaatcagaatcgaggccgggcttt

SEQ ID NO: 783 - DNA, 100 bp, Homo sapiens
  source 1..100: mol_type = genomic DNA
ctgtctcgggcattgaacaaagctaaaaactccagtgatgccaaactagaaccaacaaatgtccaaaccgtaacctgttctcctcgtgtaaaagccctgc

SEQ ID NO: 784 - DNA, 276 bp, Homo sapiens
  source 1..276: mol_type = genomic DNA
gcgatgacaacctatgcaacactccccatttacctccttgttctccaccaaagcaaggcaagaaagagaatggtccccctcactcacatacacttaagggacgaagattggtatttgacaatcagctgacaattaagtctcctagcaaaagagaactagccaaagttcaccaaaacaaaatactttcttcagttagaaaaagtcaagagatcacaacaaattctgagcagagatgtccactgaagaaagaatctgcatgtgtgagactattcaagc

SEQ ID NO: 785 - DNA, 178 bp, Homo sapiens
  source 1..178: mol_type = genomic DNA
tgctaccagcaagcaaagctggtcctgaacacagctgtcccagatcggctgcctgccagggaaagggagatggatgtcatcaggaatttcttgagggaacacatctgtgggaaaaaagctggaagcctttacctttctggtgctcctggaactggaaaaactgcctgcttaagccgga

SEQ ID NO: 786 - DNA, 101 bp, Homo sapiens
  source 1..101: mol_type = genomic DNA
tttaaaactatcatgctgaattgcatgtccttgaggactgcccaggctgtattcccagctattgctcaggagatttgtcaggaagaggtatccaggccagc

SEQ ID NO: 787 - DNA, 61 bp, Homo sapiens
  source 1..61: mol_type = genomic DNA
gacagcaaaggccaggatgtattgtacacgctatttgaatggccatggctaagcaattctc

SEQ ID NO: 788 - DNA, 135 bp, Homo sapiens
  source 1..135: mol_type = genomic DNA
gtattgctaataccctggatctcacagatagaattctacctaggcttcaagctagagaaaaatgtaagccacagctgttgaacttcccaccttataccagaaatcagatagtcactattttgcaagatcgactta

SEQ ID NO: 789 - DNA, 63 bp, Homo sapiens
  source 1..63: mol_type = genomic DNA
gtatctagagatcaggttctggacaatgctgcagttcaattctgtgcccgcaaagtctctgct

SEQ ID NO: 790 - DNA, 30 bp, Homo sapiens
  source 1..30: mol_type = genomic DNA
gagatgttcgcaaagcactggatgtttgca

SEQ ID NO: 791 - DNA, 26 bp, Homo sapiens
  source 1..26: mol_type = genomic DNA
gtgagttacggctctgttgcattctt

SEQ ID NO: 792 - DNA, 27 bp, Homo sapiens
  source 1..27: mol_type = genomic DNA
agatgtcaaaagccagactattctcaa

SEQ ID NO: 793 - DNA, 32 bp, Homo sapiens
  source 1..32: mol_type = genomic DNA
gtaaatcaccttctgagcctctgattcccaag

SEQ ID NO: 794 - DNA, 146 bp, Homo sapiens
  source 1..146: mol_type = genomic DNA
tatcccaagtcatctcagaagttgatggtaacaggatgaccttgagccaagaaggagcacaagattccttccctcttcagcagaagatcttggtttgctctttgatgctcttgatcaggcagttgaaaatcaaagaggtcactctg

SEQ ID NO: 795 - DNA, 103 bp, Homo sapiens
  source 1..103: mol_type = genomic DNA
tacagtaaagtctgtcgcaaacagcaggtggcggctgtggaccagtcagagtgtttgtcactttcagggctcttggaagccaggggcattttaggattaaaga

SEQ ID NO: 796 - DNA, 25 bp, Homo sapiens
  source 1..25: mol_type = genomic DNA
gaaatagaacatgctctgaaagata

SEQ ID NO: 797 - DNA, 1015 bp, Homo sapiens
  source 1..1015: mol_type = genomic DNA
cccgaaagtattcagctggcatttagagagctacagtcttcattttagtgctttacacattcgggcctgaaaacaaatatgaccttttttacttgaagccaatgaattttaatctatagattctttaatattagcacagaataatatctttgggtcttactatttttacccataaaagtgaccaggtagaccctttttaattacattcactacttctaccacttgtgtatctctagccaatgtgcttgcaagtgtacagatctgtgtagaggaatgtgtgtatatttacctcttcgtttgctcaaacatgagtgggtatttttttgtttgttttttttgttgttgttgtttttgaggcgcgtctcaccctgttgcccaggctggagtgcaatggcgcgttctctgctcactacagcacccgcttcccaggttgaagtgattctcttgcctcagcctcccgagtagctgggattacaggtgcccaccaccgcgcccagctaattttttaatttttagtagagacagggttttaccatgttggccaggctggtcttgaactcctgaccctcaagtgatctgcccaccttggcctccctaagtgctgggattataggcgtgagccaccatgctcagccattaaggtattttgttaagaactttaagtttagggtaagaagaatgaaaatgatccagaaaaatgcaagcaagtccacatggagatttggaggacactggttaaagaatttatttctttgtatagtatactatgttcatggtgcagatactacaacattgtggcattttagactcgttgagtttcttgggcactcccaagggcgttggggtcataaggagactataactctacagattgtgaatatatttattttcaagttgcattctttgtctttttaagcaatcagatttcaagagagctcaagctttcagaagtcaatgtgaaaattccttcctaggctgtcccacagtctttgctgcccttagatgaagccacttg

SEQ ID NO: 798 - DNA, 174 bp, Homo sapiens
  source 1..174: mol_type = genomic DNA
gccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatca

SEQ ID NO: 799 - DNA, 1072 bp, Homo sapiens
  source 1..1072: mol_type = genomic DNA
taaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggc

SEQ ID NO: 800 - DNA, 142 bp, Homo sapiens
  source 1..142: mol_type = genomic DNA
acctcgagggatgcagcttttgcgcggatgacggtggggtgctgaaccagccggtgcgcctctggaaatgtctgggcacggatcctggggccatcgacgactcctccccattcccagcaggcgggagctcttacattccgag

SEQ ID NO: 801 - DNA, 822 bp, Homo sapiens
  source 1..822: mol_type = genomic DNA
gaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgta

SEQ ID NO: 802 - DNA, 95 bp, Homo sapiens
  source 1..95: mol_type = genomic DNA
cccgccaggagttcgaagtgatggaagatcacgctgggacgtacgggttgggggacaggaaagatcaggggggctacaccatgcaccaagaccaa

SEQ ID NO: 803 - DNA, 82 bp, Homo sapiens
  source 1..82: mol_type = genomic DNA
tgtgacagcacccttagtggatgagggagctcccggcaagcaggctgccgcgcagccccacacggagatcccagaaggaacc

SEQ ID NO: 804 - DNA, 364 bp, Homo sapiens
  source 1..364: mol_type = genomic DNA
gccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaa

SEQ ID NO: 805 - DNA, 37 bp, Homo sapiens
  source 1..37: mol_type = genomic DNA
agcctggaagacgaagctgctggtcacgtgacccaag

SEQ ID NO: 806 - DNA, 143 bp, Homo sapiens
  source 1..143: mol_type = genomic DNA
ctggaattgcctgccatgacttgggggttggggggagggacatggggtgggctctgccctgaaaagatcatttggacctgagctctaattcacaagtccaggagattttagggagttggttcttatcaaaggttggctactca

SEQ ID NO: 807 - DNA, 513 bp, Homo sapiens
  source 1..513: mol_type = genomic DNA
gcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcac

SEQ ID NO: 808 - DNA, 109 bp, Homo sapiens
  source 1..109: mol_type = genomic DNA
taagctatgggaaggcctgtatacgaggggtggacttttcttctgtaagtgtccagagaccaggcctcctgaagagggcatgggggcttaacttacctggactactgtg

SEQ ID NO: 809 - DNA, 255 bp, Homo sapiens
  source 1..255: mol_type = genomic DNA
gccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctc

SEQ ID NO: 810 - DNA, 146 bp, Homo sapiens
  source 1..146: mol_type = genomic DNA
ctcatgtccggcatgcctggggctcccctcctgcctgagggccccagagaggccacacgccaaccttcggggacaggacctgaggacacagagggcggccgccacgcccctgagctgctcaagcaccagcttctaggagacctgca

SEQ ID NO: 811 - DNA, 107 bp, Homo sapiens
  source 1..107: mol_type = genomic DNA
atgaagaccgcgacgtcgatgagtcctccccccaagactcccctccctccaaggcctccccagcccaagatgggcggcctccccagacagccgccagagaagccacc

SEQ ID NO: 812 - DNA, 31 bp, Homo sapiens
  source 1..31: mol_type = genomic DNA
acgggactggaagcgatgacaaaaaagccaa

SEQ ID NO: 813 - DNA, 32 bp, Homo sapiens
  source 1..32: mol_type = genomic DNA
acatccacacgttcctctgctaaaaccttgaa

SEQ ID NO: 814 - DNA, 27 bp, Homo sapiens
  source 1..27: mol_type = genomic DNA
ctcagaccctctgatccaaccctccag

SEQ ID NO: 815 - DNA, 26 bp, Homo sapiens
  source 1..26: mol_type = genomic DNA
tcttctgtcacttcccgaactggcag

SEQ ID NO: 816 - DNA, 35 bp, Homo sapiens
  source 1..35: mol_type = genomic DNA
gggctgatggtaaaacgaagatcgccacaccgcgg

SEQ ID NO: 817 - DNA, 59 bp, Homo sapiens
  source 1..59: mol_type = genomic DNA
ccaggccaacgccaccaggattccagcaaaaaccccgcccgctccaaagacaccaccca

SEQ ID NO: 818 - DNA, 120 bp, Homo sapiens
  source 1..120: mol_type = genomic DNA
ccaagtcgccgtcttccgccaagagccgcctgcagacagcccccgtgcccatgccagacctgaagaatgtcaagtccaagatcggctccactgagaacctgaagcaccagccgggaggcg

SEQ ID NO: 819 - DNA, 71 bp, Homo sapiens
  source 1..71: mol_type = genomic DNA
agaagctggatcttagcaacgtccagtccaagtgtggctcaaaggataatatcaaacacgtcccgggaggc

SEQ ID NO: 820 - DNA, 69 bp, Homo sapiens
  source 1..69: mol_type = genomic DNA
tagtctacaaaccagttgacctgagcaaggtgacctccaagtgtggctcattaggcaacatccatcata

SEQ ID NO: 821 - DNA, 61 bp, Homo sapiens
  source 1..61: mol_type = genomic DNA
cagagtccagtcgaagattgggtccctggacaatatcacccacgtccctggcggaggaaat

SEQ ID NO: 822 - DNA, 132 bp, Homo sapiens
  source 1..132: mol_type = genomic DNA
tcgtgtacaagtcgccagtggtgtctggggacacgtctccacggcatctcagcaatgtctcctccaccggcagcatcgacatggtagactcgccccagctcgccacgctagctgacgaggtgtctgcctccc

SEQ ID NO: 823 - DNA, 224 bp, Homo sapiens
  source 1..224: mol_type = genomic DNA
ccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagta

SEQ ID NO: 824 - DNA, 28 bp, Homo sapiens
  source 1..28: mol_type = genomic DNA
acatggccacatccaacatttcctcagg

SEQ ID NO: 825 - DNA, 141 bp, Homo sapiens
  source 1..141: mol_type = genomic DNA
tgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgt

SEQ ID NO: 826 - DNA, 231 bp, Homo sapiens
  source 1..231: mol_type = genomic DNA
cctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctgg

SEQ ID NO: 827 - DNA, 442 bp, Homo sapiens
  source 1..442: mol_type = genomic DNA
ttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtc

SEQ ID NO: 828 - DNA, 264 bp, Homo sapiens
  source 1..264: mol_type = genomic DNA
ccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcacca

SEQ ID NO: 829 - DNA, 559 bp, Homo sapiens
  source 1..559: mol_type = genomic DNA
ttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgc

SEQ ID NO: 830 - DNA, 26 bp, Homo sapiens
  source 1..26: mol_type = genomic DNA
gtgtctgctgctccctagtctgggcc

SEQ ID NO: 831 - DNA, 323 bp, Homo sapiens
  source 1..323: mol_type = genomic DNA
ccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgta

SEQ ID NO: 832 - DNA, 34 bp, Homo sapiens
  source 1..34: mol_type = genomic DNA
gggtggaccgcctaagagggcgtgcgctcccgac

SEQ ID NO: 833 - DNA, 48 bp, Homo sapiens
  source 1..48: mol_type = genomic DNA
gcggcgcgccattaaccgccagatttgaatcgcgggacccgttggcag

SEQ ID NO: 834 - DNA, 27 bp, Homo sapiens
  source 1..27: mol_type = genomic DNA
tgccccgacgttgccccctgcctggca

SEQ ID NO: 835 - DNA, 48 bp, Homo sapiens
  source 1..48: mol_type = genomic DNA
tttctcaaggaccaccgcatctctacattcaagaactggcccttcttg

SEQ ID NO: 836 - DNA, 97 bp, Homo sapiens
  source 1..97: mol_type = genomic DNA
tggcttcatccactgccccactgagaacgagccagacttggcccagtgtttcttctgcttcaaggagctggaaggctgggagccagatgacgacccc

SEQ ID NO: 837 - DNA, 146 bp, Homo sapiens
  source 1..146: mol_type = genomic DNA
cctcgatgggctttgttttgaactgagttgtcaaaagatttgagttgcaaagacacttagtatgggagggttgctttccaccctcattgcttcttaaacagctgttgtgaacggatacctctctatatgctggtgccttggtgatg

SEQ ID NO: 838 - DNA, 154 bp, Homo sapiens
  source 1..154: mol_type = genomic DNA
gcttgggcaaagcactgatgccatcaacttcagacttgacgtcttactcctgaggcagagcagggtgtgcctgtggagggcgtggggaggtggcccgtggggagtggactgccgctttaatcccttcagctgcctttccgctgttgttttgatt

SEQ ID NO: 839 - DNA, 91 bp, Homo sapiens
  source 1..91: mol_type = genomic DNA
acataaaaagcattcgtccggttgcgctttcctttctgtcaagaagcagtttgaagaattaacccttggtgaatttttgaaactggacaga

SEQ ID NO: 840 - DNA, 60 bp, Homo sapiens
  source 1..60: mol_type = genomic DNA
aagaatttctgttcgaggaagagcctgatgtttgccagggtctgtttaactggacatgaa

SEQ ID NO: 841 - DNA, 49 bp, Homo sapiens
  source 1..49: mol_type = genomic DNA
agaatttgaggaaactgcggagaaagtgcgccgtgccatcgagcagctg

SEQ ID NO: 842 - DNA, 359 bp, Homo sapiens
  source 1..359: mol_type = genomic DNA
ggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttga

SEQ ID NO: 843 - DNA, 204 bp, Homo sapiens
  source 1..204: mol_type = genomic DNA
ggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaatta

SEQ ID NO: 844 - DNA, 188 bp, Homo sapiens
  source 1..188: mol_type = genomic DNA
aacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgg

SEQ ID NO: 845 - DNA, 209 bp, Homo sapiens
  source 1..209: mol_type = genomic DNA
cgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatc

SEQ ID NO: 846 - DNA, 31 bp, Homo sapiens
  source 1..31: mol_type = genomic DNA
tgttcctgagaaataaaaagcctgtcatttc

SEQ ID NO: 847 - DNA, 303 bp, Homo sapiens
  source 1..303: mol_type = genomic DNA
gtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgctta

SEQ ID NO: 848 - DNA, 46 bp, Homo sapiens
  source 1..46: mol_type = genomic DNA
ctgtgctgtgggcagggctgagctggagccgcccctctcagcccgc

SEQ ID NO: 849 - DNA, 87 bp, Homo sapiens
  source 1..87: mol_type = genomic DNA
atccttaaaaccagaccctcatggctaccagcacctgaaagcttcctcgacatctgttaataaagccgtaggcccttgtctaagtgc

SEQ ID NO: 850 - DNA, 245 bp, Homo sapiens
  source 1..245: mol_type = genomic DNA
ccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagta

SEQ ID NO: 851 - DNA, 25 bp, Homo sapiens
  source 1..25: mol_type = genomic DNA
taggaggccccccgtgtggacacag

SEQ ID NO: 852 - DNA, 32 bp, Homo sapiens
  source 1..32: mol_type = genomic DNA
tccagccgccaaatccgcaccaaggtcatgga

SEQ ID NO: 853 - DNA, 304 bp, Homo sapiens
  source 1..304: mol_type = genomic DNA
tgagctctagtgctgtcacccagtttcccttgtgaacctccttgggtggaagaagctattttctaaaccctccttagggctaggagaggcagcccccacctcttgcttctacgtggtgtctgtggcagatcctattgctgttgtggtcagcaccatgaacagggccctacagggctcttcccactgagaccactccattgggtgaatatggatggaaccagccaggtgtgagctcttaggaagctctaatctgagggcaaagactctgtctctgacctttgggagccctcgtctgaaagaaatg

SEQ ID NO: 854 - DNA, 65 bp, Homo sapiens
  source 1..65: mol_type = genomic DNA
ctggaggagaccaaaggtcgctactgcatgcagctggcccagatccaggagatgattggcagcgt

SEQ ID NO: 855 - DNA, 40 bp, Homo sapiens
  source 1..40: mol_type = genomic DNA
caagagcgagatctcggagctccggcgcaccatgcagaac

SEQ ID NO: 856 - DNA, 64 bp, Homo sapiens
  source 1..64: mol_type = genomic DNA
ggagatggacgctgcacctggcgtggacctgagccgcattctgaacgagatgcgtgaccagtat

SEQ ID NO: 857 - DNA, 38 bp, Homo sapiens
  source 1..38: mol_type = genomic DNA
gatgggtgtctcataccttttctctggggtcattccag

SEQ ID NO: 858 - DNA, 75 bp, Homo sapiens
  source 1..75: mol_type = genomic DNA
agccacagtggacaatgccaatgtccttctgcagattgacaatgcccgtctggccgcggatgacttccgcaccaa

SEQ ID NO: 859 - DNA, 50 bp, Homo sapiens
  source 1..50: mol_type = genomic DNA
tggccttggtgctggcttgggtggtggctttggtggtggctttgctggtg

SEQ ID NO: 860 - DNA, 44 bp, Homo sapiens
  source 1..44: mol_type = genomic DNA
acccgagcaccttctcttcactcagccaactgctcgctcgctca

SEQ ID NO: 861 - DNA, 39 bp, Homo sapiens
  source 1..39: mol_type = genomic DNA
gagaaggtgaccatgcagaacctcaatgaccgcctggcc

SEQ ID NO: 862 - DNA, 25 bp, Homo sapiens
  source 1..25: mol_type = genomic DNA
ggactcagctaccccggccggccac

SEQ ID NO: 863 - DNA, 25 bp, Homo sapiens
  source 1..25: mol_type = genomic DNA
ccaggaatacaaaatcctgctggat

SEQ ID NO: 864 - DNA, 30 bp, Homo sapiens
  source 1..30: mol_type = genomic DNA
aagagtgagatctcggagctccggcgcacc

SEQ ID NO: 865 - DNA, 80 bp, Homo sapiens
  source 1..80: mol_type = genomic DNA
aggtgggtggtgagatcaatgtggagatggacgctgccccaggcgtggacctgagccgcatcctcaacgagatgcgtgac

SEQ ID NO: 866 - DNA, 121 bp, Homo sapiens
  source 1..121: mol_type = genomic DNA
gtgcaccgggattagtcaccttagagggcttccctgtctgcagagccctgatccttggggtccagtgtgcagggcagactcctctttgtaccacactgcttctctgtacacaaggaacctc

SEQ ID NO: 867 - DNA, 45 bp, Homo sapiens
  source 1..45: mol_type = genomic DNA
ctggccagagccgacctggagatgcagattgagaacctcaaggag

SEQ ID NO: 868 - DNA, 38 bp, Homo sapiens
  source 1..38: mol_type = genomic DNA
ccctgcgcctgagtgtggaggccgacatcaatggcctg

SEQ ID NO: 869 - DNA, 25 bp, Homo sapiens
  source 1..25: mol_type = genomic DNA
gtctggctgctgatgacttccgcac

SEQ ID NO: 870 - DNA, 25 bp, Homo sapiens
  source 1..25: mol_type = genomic DNA
ggtactgagtatcgggggaagaaga

SEQ ID NO: 871 - DNA, 60 bp, Homo sapiens
  source 1..60: mol_type = genomic DNA
ggcagcagctttgggggtgttgatgggctgctggctggaggtgagaaggccaccatgcag

SEQ ID NO: 872 - DNA, 131 bp, Homo sapiens
  source 1..131: mol_type = genomic DNA
gtcccgcacctcctgccggctgtctggcggcctgggtgccggctcctgcaggctgggatctgctggcggcctgggcagcaccctcgggggtagcagctactccagctgctacagctttggctctggtggtg

SEQ ID NO: 873 - DNA, 33 bp, Homo sapiens
  source 1..33: mol_type = genomic DNA
acacgcacggcactcagcacgaggatttggaga

SEQ ID NO: 874 - DNA, 28 bp, Homo sapiens
  source 1..28: mol_type = genomic DNA
ttggcctgcctccgtcccgccgcgccac

SEQ ID NO: 875 - DNA, 25 bp, Homo sapiens
  source 1..25: mol_type = genomic DNA
ctgcctccgtcccgccgcgccactt

SEQ ID NO: 876 - DNA, 26 bp, Homo sapiens
  source 1..26: mol_type = genomic DNA
gcctgtggccggctcggagctgccgc

SEQ ID NO: 877 - DNA, 318 bp, Homo sapiens
  source 1..318: mol_type = genomic DNA
cgcttcgcagcgttttcaaaaactggagcgaaagtgatgtgggcggggcaaaggcggcgggaagaggagagcactgaagctggcgcgggaacttggtttcctggtggcctcccatccaatccccacgaaccagctttcctcttaaaccttgaaaagagaaattcgggagttcgagtataagttcttagtcgtcctttcctctttcctttccgacaggagcaccccaggcaaaaaatgtctcgcgggtcattggcgccaggctttcaggggacagtggggcggggcggggtgggcacaggacgttaggcagccgttggc

SEQ ID NO: 878 - DNA, 33 bp, Homo sapiens
  source 1..33: mol_type = genomic DNA
cgtcctgccgtcctggatcctgcgccagctgcg

SEQ ID NO: 879 - DNA, 38 bp, Homo sapiens
  source 1..38: mol_type = genomic DNA
ctctgctgacaaccaaacgtgtgttctggaagggtgtt

SEQ ID NO: 880 - DNA, 79 bp, Homo sapiens
  source 1..79: mol_type = genomic DNA
gctgtcttccaagggagtgaaaatctgggatgccaatggatcccgagactttttggacagcctgggattctccaccaga

SEQ ID NO: 881 - DNA, 73 bp, Homo sapiens
  source 1..73: mol_type = genomic DNA
aggacagggagttgaccaactgcaaagagtgattgacaccatcaaaaccaaccctgacgacagaagaatcatc

SEQ ID NO: 882 - DNA, 87 bp, Homo sapiens
  source 1..87: mol_type = genomic DNA
aagcaatctggttttgtgcagaggcacctgagggaggcaggaccctgggaacttcccccagccacatggttgattgtgtgacgttgg

SEQ ID NO: 883 - DNA, 140 bp, Homo sapiens
  source 1..140: mol_type = genomic DNA
ctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaa

SEQ ID NO: 884 - DNA, 45 bp, Homo sapiens
  source 1..45: mol_type = genomic DNA
ggagatgcacatatttacctgaatcacatcgagccactgaaaatt

SEQ ID NO: 885 - DNA, 101 bp, Homo sapiens
  source 1..101: mol_type = genomic DNA
ccaaagctcaggattcttcgaaaagttgagaaaattgatgacttcaaagctgaagactttcagattgaagggtacaatccgcatccaactattaaaatgga

SEQ ID NO: 886 - DNA, 121 bp, Homo sapiens
  source 1..121: mol_type = genomic DNA
aggagctcgaaggatattgtcagtctttaggggttgggctggatgccgaggtaaaagttctttttgctctaaaagaaaaaggaactaggtcaaaaatctgtccgtgacctatcagttatta

SEQ ID NO: 887 - DNA, 235 bp, Homo sapiens
  source 1..235: mol_type = genomic DNA
cactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggtta

SEQ ID NO: 888 - DNA, 40 bp, Homo sapiens
  source 1..40: mol_type = genomic DNA
ttgttcattctgtactgccacttatctgctcagttccttc

SEQ ID NO: 889 - DNA, 117 bp, Homo sapiens
  source 1..117: mol_type = genomic DNA
agcgccggcggagaatttcaaattcgaacggctttggcgggccgaggaaggacctggtgttttgatgaccgctgtcctgtctagcagatacttgcacggtttacagaaattcggtcc

SEQ ID NO: 890 - DNA, 98 bp, Homo sapiens
  source 1..98: mol_type = genomic DNA
atgaagcgcagttcagtttccagcggtggtgctggccgcctctccatgcaggagttaagatcccaggatgtaaataaacaaggcctctatacccctca

SEQ ID NO: 891 - DNA, 93 bp, Homo sapiens
  source 1..93: mol_type = genomic DNA
gtggacatggatcccggaatagtcaacttggtatattttccagttctgagaaaatcaaggacccgagaccacttaatgacaaagcattcattc

SEQ ID NO: 892 - DNA, 133 bp, Homo sapiens
  source 1..133: mol_type = genomic DNA
cttacagaaaatggttatgcacataatgtgtccatgaaatctctacaagctccctctgttaaagacttcctgaagatcttcacatttctttatggcttcctgtgcccctcatacgaacttcctgacacaaagt

SEQ ID NO: 893 - DNA, 59 bp, Homo sapiens
  source 1..59: mol_type = genomic DNA
ctcctcatacatggcctcacattgtggcagccttagtttggctaatagactgcatcaag

SEQ ID NO: 894 - DNA, 123 bp, Homo sapiens
  source 1..123: mol_type = genomic DNA
tgaaatgtatacatgggaaagggtttttttcctcaaaaaaaatattttctctcccagtcttttgacagtattctcaaagtctgcttcagagttttcatttttcaaagcacatttgattttaag

SEQ ID NO: 895 - DNA, 33 bp, Homo sapiens
  source 1..33: mol_type = genomic DNA
atgaaagaaagctcacctttatttgatgatggg

SEQ ID NO: 896 - DNA, 57 bp, Homo sapiens
  source 1..57: mol_type = genomic DNA
ctacaccataaaatgctatgagagttttatgagtggtgccgacagctttgatgagat

SEQ ID NO: 897 - DNA, 71 bp, Homo sapiens
  source 1..71: mol_type = genomic DNA
gcttttaagctggaatcattagaagcaaaaaacagagcattgaatgaacagattgcaagattggaacaaga

SEQ ID NO: 898 - DNA, 137 bp, Homo sapiens
  source 1..137: mol_type = genomic DNA
aatcgtctagagtcgttgagaaaactgaaggcttccttacaaggagatgttcaaaagtatcaggcatacatgagcaatttggagtctcattcagccattcttgaccagaaattaaatggtctcaatgaggaaattgc

SEQ ID NO: 899 - DNA, 126 bp, Homo sapiens
  source 1..126: mol_type = genomic DNA
aaccagaagtactcagttgcagacattgagcgaataaatcatgaaagaaatgaattgcagcagactattaataaattaaccaaggacctggaagctgaacaacagaagttgtggaatgaggagtta

SEQ ID NO: 900 - DNA, 123 bp, Homo sapiens
  source 1..123: mol_type = genomic DNA
cagagtatcacaaattggctagaaaattaaaacttattcctaaaggtgctgagaattccaaaggttatgactttgaaattaagtttaatcccgaggctggtgccaactgccttgtcaaataca

SEQ ID NO: 901 - DNA, 89 bp, Homo sapiens
  source 1..89: mol_type = genomic DNA
gtacctcttaaggaactcctgaatgaaactgaagaagaaattaataaagccctaaataaaaaaatgggtttggaggatactttagaaca

SEQ ID NO: 902 - DNA, 87 bp, Homo sapiens
  source 1..87: mol_type = genomic DNA
ttgaatgcaatgataacagaaagcaagagaagtgtgagaactctgaaagaagaagttcaaaagctggatgatctttaccaacaaaaa

SEQ ID NO: 903 - DNA, 63 bp, Homo sapiens
  source 1..63: mol_type = genomic DNA
acctgctagaaagtactgttaaccaggggctcagtgaagctatgaatgaattagatgctgttc

SEQ ID NO: 904 - DNA, 84 bp, Homo sapiens
  source 1..84: mol_type = genomic DNA
tagttgtgcaaaccacgactgaagaaagacgaaaagtgggaaataacttgcaacgtctgttagagatggttgctacacatgttg

SEQ ID NO: 905 - DNA, 279 bp, Homo sapiens
  source 1..279: mol_type = genomic DNA
ctggggtgaagcagccgcatgctaaggaacaccaaggactgccaggagccgccagcaactggggagagacgaagaaggattcttccctagagccttcagagagaccatggccctgctgacgtcttgatttcaaacttccggcctccagagctgaaagagtacatttctgttgttttaagccacctagtttgtggcaatttgttacagtatcagtatttgaaatcgcaaaaaaatcaacaaaaacaacaagaaaaaataatgtggcatgttagtttccca

SEQ ID NO: 906 - DNA, 132 bp, Homo sapiens
  source 1..132: mol_type = genomic DNA
aacatcttgaggagcagattgctaaagttgatagagaatatgaagaatgcatgtcagaagatctctcggaaaatattaaagagattagagataagtatgagaagaaagctactctaattaagtcttctgaag

SEQ ID NO: 907 - DNA, 33 bp, Homo sapiens
  source 1..33: mol_type = genomic DNA
tatatccatagtgaataaaattgtctcagtaaa

SEQ ID NO: 908 - DNA, 303 bp, Homo sapiens
  source 1..303: mol_type = genomic DNA
caaaatgttcgtgcgggtatataccagatgagtacagtgagtagttttatgtatcaccagactgggttattgccaagttatatatcaccaaaagctgtatgactggatgttctggttacctggtttacaaaattatcagagtagtaaaactttgatatatatgaggatattaaaactacactaagtatcatttgattcgattcagaaagtactttgatatctctcagtgcttcagtgctatcattgtgagcaattgtcttttatatacggtactgtagccatactaggcctgtctgtggcatt

SEQ ID NO: 909 - DNA, 28 bp, Homo sapiens
  source 1..28: mol_type = genomic DNA
agaactgctggtgtttaggaataagaat

SEQ ID NO: 910 - DNA, 183 bp, Homo sapiens
  source 1..183: mol_type = genomic DNA
agtttcagtaggtcatagggagatgagtttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtatta

SEQ ID NO: 911 - DNA, 86 bp, Homo sapiens
  source 1..86: mol_type = genomic DNA
agaatgctgggatgcttttgggttttggaattatgttacttatttccatatttgaacataaaatcgtgtttcgtataaatttctag

SEQ ID NO: 912 - DNA, 42 bp, Homo sapiens
  source 1..42: mol_type = genomic DNA
atgctgcacaatgatgctagtgaccatggatgtagccgctgg

SEQ ID NO: 913 - DNA, 164 bp, Homo sapiens
  source 1..164: mol_type = genomic DNA
gcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatg

SEQ ID NO: 914 - DNA, 35 bp, Homo sapiens
  source 1..35: mol_type = genomic DNA
gttgctgtgttctgtcatgagttgcctcatgaatt

SEQ ID NO: 915 - DNA, 88 bp, Homo sapiens
  source 1..88: mol_type = genomic DNA
gagctgaaagatgccggcgtcgccactctggcctggatggtgataatgggtgatggcctgcacaatttcagcgatggcctagcaattg

SEQ ID NO: 916 - DNA, 74 bp, Homo sapiens
  source 1..74: mol_type = genomic DNA
accatcatattctccatcatcaccaccaccaaaaccaccatcctcacagtcacagccagcgctactctcgggag

SEQ ID NO: 917 - DNA, 101 bp, Homo sapiens
  source 1..101: mol_type = genomic DNA
ccacaggaagtctacaatgaatatgtacccagagggtgcaagaataaatgccattcacatttccacgatacactcggccagtcagacgatctcattcacca

SEQ ID NO: 918 - DNA, 62 bp, Homo sapiens
  source 1..62: mol_type = genomic DNA
tgatgtggagattaagaagcagttgtccaagtatgaatctcaactttcaacaaatgaggaga

SEQ ID NO: 919 - DNA, 98 bp, Homo sapiens
  source 1..98: mol_type = genomic DNA
tgcctattttgattccacgtggaagggtctaacagctctaggaggcctgtatttcatgtttcttgttgaacatgtcctcacattgatcaaacaattta

SEQ ID NO: 920 - DNA, 26 bp, Homo sapiens
  source 1..26: mol_type = genomic DNA
tcagtcatctgtcttctcaaaacata

SEQ ID NO: 921 - DNA, 138 bp, Homo sapiens
  source 1..138: mol_type = genomic DNA
gttttatagccatttccatcatcagtttcctgtctctgctgggggttatcttagtgcctctcatgaatcgggtgtttttcaaatttctcctgagtttccttgtggcactggccgttgggactttgagtggtgatgctt

SEQ ID NO: 922 - DNA, 120 bp, Homo sapiens
  source 1..120: mol_type = genomic DNA
gaatgcaacagagttcaactatctctgtccagccatcatcaaccaaattgatgctagatcttgtctgattcatacaagtgaaaagaaggctgaaatccctccaaagacctattcattaca

SEQ ID NO: 923 - DNA, 27 bp, Homo sapiens
  source 1..27: mol_type = genomic DNA
gcatcaaagctactgacatctcatggc

SEQ ID NO: 924 - DNA, 63 bp, Homo sapiens
  source 1..63: mol_type = genomic DNA
tgagccggctggctggtaggaaaacaaatgaatctgtgagtgagccccgaaaaggctttatgt

SEQ ID NO: 925 - DNA, 245 bp, Homo sapiens
  source 1..245: mol_type = genomic DNA
actcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgg

SEQ ID NO: 926 - DNA, 201 bp, Homo sapiens
  source 1..201: mol_type = genomic DNA
cgcaatggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggaga

SEQ ID NO: 927 - DNA, 56 bp, Homo sapiens
  source 1..56: mol_type = genomic DNA
tgtggaaccaaacctgcgcgcgtggccgggccgtgggacaacgaggccgcggagac

SEQ ID NO: 928 - DNA, 28 bp, Homo sapiens
  source 1..28: mol_type = genomic DNA
agatttctcgaagacaccagtgggcccg

SEQ ID NO: 929 - DNA, 134 bp, Homo sapiens
  source 1..134: mol_type = genomic DNA
ctgcgcgcggcggtaattagtgattgtcttccagcttcgcgaaggctaggggcgcggctgccgggtggctgcgcggcgctgcccccggaccgaggggcagccaacccaatgaaaccaccgcgtgttcgcgcctg

SEQ ID NO: 930 - DNA, 26 bp, Homo sapiens
  source 1..26: mol_type = genomic DNA
tgaagaaaaataaagtacagtgtgag

SEQ ID NO: 931 - DNA, 239 bp, Homo sapiens
  source 1..239: mol_type = genomic DNA
tgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtc

SEQ ID NO: 932 - DNA, 112 bp, Homo sapiens
  source 1..112: mol_type = genomic DNA
ctgtagtgtagatactgagtaaatccatgcacctaaaccttttggaaaatctgccgtgggccctccagatagctcatttcattaagtttttccctccaaggtagaatttgca

SEQ ID NO: 933 - DNA, 35 bp, Homo sapiens
  source 1..35: mol_type = genomic DNA
ggaggatggaaaggctcgctcaatcaagaaaattc

SEQ ID NO: 934 - DNA, 156 bp, Homo sapiens
  source 1..156: mol_type = genomic DNA
gaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgag

SEQ ID NO: 935 - DNA, 1832 bp, Homo sapiens
  source 1..1832: mol_type = genomic DNA
cctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatc

SEQ ID NO: 936 - DNA, 274 bp, Homo sapiens
  source 1..274: mol_type = genomic DNA
cgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagc

SEQ ID NO: 937 - DNA, 410 bp, Homo sapiens
  source 1..410: mol_type = genomic DNA
taggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcatt

SEQ ID NO: 938 - DNA, 39 bp, Homo sapiens
  source 1..39: mol_type = genomic DNA
caacagggcagtgtggtctccgaatgtctggaagctgat

SEQ ID NO: 939 - DNA, 639 bp, Homo sapiens
  source 1..639: mol_type = genomic DNA
cacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtc

SEQ ID NO: 940 - DNA, 41 bp, Homo sapiens
  source 1..41: mol_type = genomic DNA
gtgggagcttgcatcaccctgggtgcctatctgggccacaa

SEQ ID NO: 941 - DNA, 81 bp, Homo sapiens
  source 1..81: mol_type = genomic DNA
tgtggaactgtacggccccagcatgcggcctctgtttgatttctcctggctgtctctgaagactctgctcagtttggccct

SEQ ID NO: 942 - DNA, 495 bp, Homo sapiens
  source 1..495: mol_type = genomic DNA
gcaataccattctcatgccagtgtacaaattacatgaaagagcatcatttttctagtgtctgaggattggctgcttatggccaattttggcagcaagacgataggattaaaaatagcttgaagatgatctagtcttaaataatatatttcatgatgaactttccttgggaaagtgcatctttctgcctacaagaatcacatgacccctttcaataatttatgtagtagagaaaaacacactatttctcatagagttttcagtcatgtgctgtggtgtgattgtttctggacattcataaaattttatagttaactgaattctcttttctgttttgttgctatttaacgtccattgaaaacatggctttcttttgcgcattctgttactttcagctgtactttctaataagaatggattgccctttttagcaatctttgattgaactggtacatttcagattacttaaatgtcatcaggccacacagcataccagg

SEQ ID NO: 943 - DNA, 210 bp, Homo sapiens
  source 1..210: mol_type = genomic DNA
acttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacc

SEQ ID NO: 944 - DNA, 25 bp, Homo sapiens
  source 1..25: mol_type = genomic DNA
gccacctgtggtccacctgaccctc

SEQ ID NO: 945 - DNA, 86 bp, Homo sapiens
  source 1..86: mol_type = genomic DNA
cccgcaccgggcatcttctcctcccagcccgggcacacgccccatccagccgcatcccgggacccggtcgccaggacctcgccgct

SEQ ID NO: 946 - DNA, 63 bp, Homo sapiens
  source 1..63: mol_type = genomic DNA
gtacgataaccgggagatagtgatgaagtacatccattataagctgtcgcagaggggctacga

SEQ ID NO: 947 - DNA, 212 bp, Homo sapiens
  source 1..212: mol_type = genomic DNA
cacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttcttt

SEQ ID NO: 948 - DNA, 154 bp, Homo sapiens
  source 1..154: mol_type = genomic DNA
tgtaactttcaatggaaacctttgagattttttacttaaagtgcattcgagtaaatttaatttccaggcagcttaatacattctttttagccgtgttacttgtagtgtgtatgccctgctttcactcagtgtgtacagggaaacgcacctgatt

SEQ ID NO: 949 - DNA, 419 bp, Homo sapiens
  source 1..419: mol_type = genomic DNA
ggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaa

SEQ ID NO: 950 - DNA, 27 bp, Homo sapiens
  source 1..27: mol_type = genomic DNA
ggaggcggccgtagccagcgccgccgc

SEQ ID NO: 951 - DNA, 38 bp, Homo sapiens
  source 1..38: mol_type = genomic DNA
tgccggggctccgggccctccctgccggcggccgtcag

SEQ ID NO: 952 - DNA, 31 bp, Homo sapiens
  source 1..31: mol_type = genomic DNA
ccgccgctctccgtggccccgccgcgctgcc

SEQ ID NO: 953 - DNA, 25 bp, Homo sapiens
  source 1..25: mol_type = genomic DNA
ttttaaatgtcccgctctgagccgg

SEQ ID NO: 954 - DNA, 39 bp, Homo sapiens
  source 1..39: mol_type = genomic DNA
gacggcggcgcggagttctcggctcgctccaggaagagg

SEQ ID NO: 955 - DNA, 46 bp, Homo sapiens
  source 1..46: mol_type = genomic DNA
gaaatggccaaaatcgacaggacggcgagggaccagtgtgggagcc

SEQ ID NO: 956 - DNA, 140 bp, Homo sapiens
  source 1..140: mol_type = genomic DNA
tctaatgcagccacagcccatatggcccagcactgtacctgtcagtgggcactggcctctgccagtcctgggattccaggaagcttggtgttcctgactggcaccgtctgagattacagatatgtgcctagcctggaaga

SEQ ID NO: 957 - DNA, 68 bp, Homo sapiens
  source 1..68: mol_type = genomic DNA
agaagatgatgaccgggtttacccaaactcaacgtgcaagcctcggattattgcaccatccagaggct

SEQ ID NO: 958 - DNA, 83 bp, Homo sapiens
  source 1..83: mol_type = genomic DNA
aaagacatacttaagggatcagcactttcttgagcaacaccctcttctgcagccaaaaatgcgagcaattcttctggattggt

SEQ ID NO: 959 - DNA, 102 bp, Homo sapiens
  source 1..102: mol_type = genomic DNA
gggagaccttttacttggcacaagatttctttgaccggtatatggcgacacaagaaaatgttgtaaaaactcttttacagcttattgggatttcatctttat

SEQ ID NO: 960 - DNA, 83 bp, Homo sapiens
  source 1..83: mol_type = genomic DNA
gaaatctatcctccaaagttgcaccagtttgcgtatgtgacagatggagcttgttcaggagatgaaattctcaccatggaatt

SEQ ID NO: 961 - DNA, 99 bp, Homo sapiens
  source 1..99: mol_type = genomic DNA
tggcgtttaagtcccctgactattgtgtcctggctgaatgtatacatgcaggttgcatatctaaatgacttacatgaagtgctactgccgcagtatccc

SEQ ID NO: 962 - DNA, 88 bp, Homo sapiens
  source 1..88: mol_type = genomic DNA
ctggatgttgactgccttgaatttccttatggtatacttgctgcttcggccttgtatcatttctcgtcatctgaattgatgcaaaagg

SEQ ID NO: 963 - DNA, 103 bp, Homo sapiens
  source 1..103: mol_type = genomic DNA
ctgtgtcaagtggatggttccatttgccatggttataagggagacggggagctcaaaactgaagcacttcaggggcgtcgctgatgaagatgcacacaacata

SEQ ID NO: 964 - DNA, 55 bp, Homo sapiens
  source 1..55: mol_type = genomic DNA
gacaaagcccgagcaaagaaagccatgttgtctgaacaaaatagggcttctcctc

SEQ ID NO: 965 - DNA, 35 bp, Homo sapiens
  source 1..35: mol_type = genomic DNA
gtaagaagcagagcagcgggccggaaatggcgtga

SEQ ID NO: 966 - DNA, 215 bp, Homo sapiens
  source 1..215: mol_type = genomic DNA
gcgtgcgtttgcttttacagatatctgaatggaagagtgtttcttccacaacagaagtatttctgtggatggcatcaaacagggcaaagtgttttttattgaatgcttataggttttttttaaataagtgggtcaagtacaccagccacctccagacaccagtgcgtgctcccgatgctgctatggaaggtgctacttgacctaagggactccca

SEQ ID NO: 967 - DNA, 130 bp, Homo sapiens
  source 1..130: mol_type = genomic DNA
tgggctccgttgtaccaagtggagcaggtggttgcgggcaagcgttgtgcagagcccatagccagctgggcagggggctgccctctccacattatcagttgacagtgtacaatgcctttgatgaactgtt

SEQ ID NO: 968 - DNA, 36 bp, Homo sapiens
  source 1..36: mol_type = genomic DNA
gtaagtgctgctatatctatccattttttaataaag

SEQ ID NO: 969 - DNA, 88 bp, Homo sapiens
  source 1..88: mol_type = genomic DNA
tctaggtggtgtgggcgaagtttgggactggtttagggcggggacaagaccaagaacacaagtttccttgtactacgggagagaggga

SEQ ID NO: 970 - DNA, 39 bp, Homo sapiens
  source 1..39: mol_type = genomic DNA
ggtccctggtgtgccttggtgtcatcatcttgctgtctg

SEQ ID NO: 971 - DNA, 58 bp, Homo sapiens
  source 1..58: mol_type = genomic DNA
ttcaggactacatggcccccgactgccgattcctgaccattcaccggggccaagtggt

SEQ ID NO: 972 - DNA, 25 bp, Homo sapiens
  source 1..25: mol_type = genomic DNA
gccgtgggcggctcttctggggagg

SEQ ID NO: 973 - DNA, 134 bp, Homo sapiens
  source 1..134: mol_type = genomic DNA
catttaagctgagattcatatgacaaggatggagcagttatgtggagatcagggagaagggagaatgcaaaggccttcagcaggcacaagcttgccatcttcccagaccctagcttttaactcctcttccccag

SEQ ID NO: 974 - DNA, 79 bp, Homo sapiens
  source 1..79: mol_type = genomic DNA
gttcagggagattactatggagatctggctgctcgcctgggctatttccccagtagcattgtccgagaggaccagaccc

SEQ ID NO: 975 - DNA, 92 bp, Homo sapiens
  source 1..92: mol_type = genomic DNA
aaagtgcttcaacccgcgccggcggcgactgcagttcctgcgagcgaggagcgcgggacctgctgacacgctgacgccttcgagcgcggccc

SEQ ID NO: 976 - DNA, 78 bp, Homo sapiens
  source 1..78: mol_type = genomic DNA
gctgcactaccaggacacagattcagatgtgccggagcagagggatagcaagtgcaaggtcaaatggacccatgagga

SEQ ID NO: 977 - DNA, 54 bp, Homo sapiens
  source 1..54: mol_type = genomic DNA
ccctggtgaggcagtttggacagcaggactggaagttcctggccagccacttcc

SEQ ID NO: 978 - DNA, 36 bp, Homo sapiens
  source 1..36: mol_type = genomic DNA
tccagaccttgtcaaggggccatggaccaaagagga

SEQ ID NO: 979 - DNA, 176 bp, Homo sapiens
  source 1..176: mol_type = genomic DNA
tgattgccaagcacctgaagggccggctggggaagcagtgccgtgaacgctggcacaaccacctcaaccctgaggtgaagaagtcttgctggaccgaggaggaggaccgcatcatctgcgaggcccacaaggtgctgggcaaccgctgggccgagatcgccaagatgttgccaggg

SEQ ID NO: 980 - DNA, 42 bp, Homo sapiens
  source 1..42: mol_type = genomic DNA
acacaggaggcttcttgagcgagtccaaagactgcaagcccc

SEQ ID NO: 981 - DNA, 194 bp, Homo sapiens
  source 1..194: mol_type = genomic DNA
gcagccaccacatcgaaggaacaggagcccatcggtacagatctggacgcagtgcgaacaccagagcccttggaggaattcccgaagcgtgaggaccaggaaggctccccaccagaaacgagcctgccttacaagtgggtggtggaggcagctaacctcctcatccctgctgtgggttctagcctctctgaagc

SEQ ID NO: 982 - DNA, 43 bp, Homo sapiens
  source 1..43: mol_type = genomic DNA
ctgatgcttggtgtgacctgagtaaatttgacctccctgagga

SEQ ID NO: 983 - DNA, 52 bp, Homo sapiens
  source 1..52: mol_type = genomic DNA
gtatcaacaacagcctagtgcagctgcaagcgtcacatcagcagcaagtcct

SEQ ID NO: 984 - DNA, 233 bp, Homo sapiens
  source 1..233: mol_type = genomic DNA
cagtgtgaccgagtaccgcctggatggccacaccatctcagacctgagccggagcagccggggcgagctgatccccatctcccccagcactgaagtcgggggctctggcattggcacaccgccctctgtgctcaagcggcagaggaagaggcgtgtggctctgtcccctgtcactgagaatagcaccagtctgtccttcctggattcctgtaacagcctcacgcccaagagca

SEQ ID NO: 985 - DNA, 104 bp, Homo sapiens
  source 1..104: mol_type = genomic DNA
tttctgaacttctggaacaaacaggacacattggagctggagagcccctcgctgacatccaccccagtgtgcagccagaaggtggtggtcaccacaccactgca

SEQ ID NO: 986 - DNA, 72 bp, Homo sapiens
  source 1..72: mol_type = genomic DNA
ctccatggacaacactccccacacgccaaccccgttcaagaacgccctggagaagtacggacccctgaagcc

SEQ ID NO: 987 - DNA, 72 bp, Homo sapiens
  source 1..72: mol_type = genomic DNA
acttgaaggaggtgctgcgttctgaggctggcatcgaactcatcatcgaggacgacatcaggcccgagaagc

SEQ ID NO: 988 - DNA, 29 bp, Homo sapiens
  source 1..29: mol_type = genomic DNA
ccgacaactgccccttcaaactcttccag

SEQ ID NO: 989 - DNA, 31 bp, Homo sapiens
  source 1..31: mol_type = genomic DNA
cctcaccctgtcaggtatcaaagaagacaac

SEQ ID NO: 990 - DNA, 86 bp, Homo sapiens
  source 1..86: mol_type = genomic DNA
cttgctcaaccagggcttcttgcaggccaagcccgagaaggcagcagtggcccagaagccccgaagccacttcacgacacctgccc

SEQ ID NO: 991 - DNA, 34 bp, Homo sapiens
  source 1..34: mol_type = genomic DNA
cagccacacatctcggaccctcatcttgtcctga

SEQ ID NO: 992 - DNA, 265 bp, Homo sapiens
  source 1..265: mol_type = genomic DNA
gtgtcacgagcccattctcatgtttacaggggttgtgggggcagagggggtctgtgaatctgagagtcattcaggtgacctcctgcagggagccttctgccaccagcccctccccagactctcaggtggaggcaacagggccatgtgctgccctgttgccgagcccagctgtgggcggctcctggtgctaacaacaaagttccacttccaggtctgcctggttccctccccaaggccacagggagctccgtcagcttctcccaag

SEQ ID NO: 993 - DNA, 54 bp, Homo sapiens
  source 1..54: mol_type = genomic DNA
gtcctgcagttgcagtcgtgttctccgagttcctgtctctctgccaacgccgcc

SEQ ID NO: 994 - DNA, 66 bp, Homo sapiens
  source 1..66: mol_type = genomic DNA
tggcttcccaaaaccgcgacccagccgccactagcgtcgccgccgcccgtaaaggagctgagccga

SEQ ID NO: 995 - DNA, 252 bp, Homo sapiens
  source 1..252: mol_type = genomic DNA
gagctcagaccgctctttgagactctcccgaaggagaatgggagggtaggggcgctgccagactccttccctggtgggcctagatgaagacgctcaaggaccctcgtgacttggccgagacaggggaagggagaagttgagtcgggcaaggaagagatgctaaagcctggggaattaagaacatgccagaatcatcccgagggagtctggaattagggagggtgaggactcgctaggatcgtcctgtggatc

SEQ ID NO: 996 - DNA, 52 bp, Homo sapiens
  source 1..52: mol_type = genomic DNA
atgtctggcgataaagggatttctgccttccctgaatcagacaaccttttca

SEQ ID NO: 997 - DNA, 25 bp, Homo sapiens
  source 1..25: mol_type = genomic DNA
cagtttgtctactgtccggtcccag

SEQ ID NO: 998 - DNA, 34 bp, Homo sapiens
  source 1..34: mol_type = genomic DNA
actcaagattctagcaagccccttgtgtggggct

SEQ ID NO: 999 - DNA, 91 bp, Homo sapiens
  source 1..91: mol_type = genomic DNA
ggtataagctctcgctagagttccccagtggctacccttacaatgcgcccacagtgaagttcctcacgccctgctatcaccccaacgtgga

SEQ ID NO: 1000 - DNA, 33 bp, Homo sapiens
  source 1..33: mol_type = genomic DNA
acacacatgctgccgagctctggaaaaacccca

SEQ ID NO: 1001 - DNA, 43 bp, Homo sapiens
  source 1..43: mol_type = genomic DNA
gtacctgcaagaaacctactcaaagcaggtcaccagccaggag

SEQ ID NO: 1002 - DNA, 36 bp, Homo sapiens
  source 1..36: mol_type = genomic DNA
gtgatttctgtataggactctttatcttgagctgtg

SEQ ID NO: 1003 - DNA, 116 bp, Homo sapiens
  source 1..116: mol_type = genomic DNA
ggcctttacgcgacatccgagcagcgtgtctatcccaaaggcctaggagcatttgcccggctcggtcaaatctagcgcaagtttgaagcctgcggcctcgcaattttagcagcttc

SEQ ID NO: 1004 - DNA, 146 bp, Homo sapiens
  source 1..146: mol_type = genomic DNA
gtggagtcttcttgaataagctgtgaaacatttccccacccgcttccctttcttggcccaggcttcctgaccacagcctcacctttgagcagctcagagccctgcctgccaggatgcgagccactgcctggatcgtggctctgcag

SEQ ID NO: 1005 - DNA, 32 bp, Homo sapiens
  source 1..32: mol_type = genomic DNA
ggcccggagcggcccagcaagcccagcagccc

SEQ ID NO: 1006 - DNA, 53 bp, Homo sapiens
  source 1..53: mol_type = genomic DNA
ggctccggccgcctggctccgcagcgcggccgcgcgcgccctcctgcccccga

SEQ ID NO: 1007 - DNA, 33 bp, Homo sapiens
  source 1..33: mol_type = genomic DNA
catgcagccctgcccagtagcccggcacctgcc

SEQ ID NO: 1008 - DNA, 29 bp, Homo sapiens
  source 1..29: mol_type = genomic DNA
gaagccccccggcctgccagcagcctcag

SEQ ID NO: 1009 - DNA, 54 bp, Homo sapiens
  source 1..54: mol_type = genomic DNA
ctgtggcgtgcccgacccatctgatgggctgagtgcccgcaaccgacagaagag

SEQ ID NO: 1010 - DNA, 30 bp, Homo sapiens
  source 1..30: mol_type = genomic DNA
gcgctgggagaagacggacctcacctacag

SEQ ID NO: 1011 - DNA, 595 bp, Homo sapiens
  source 1..595: mol_type = genomic DNA
cagggaggtcatctatgggcaaaccccctgaaaccccaacttagacacatacacatatggagaccctccctcagcagaggggcagagcctccgtcatcatgcaaagagtcgcagcacatgcctgcggacgggtgttcagtcactcaggcagcctttacaagagacctgtgaggaccaggctctgggactccacggtgaatgaggcagacacagccccatcctctgtgtcagtctgaggtgggtgtcagccatgtcattgtccaactctaccatcacaacttgggcttcgagcaggtggagacagtggtaagcggggagaggcaatagtgggcatctcactgggtgacctgggaggaccctgggcaggtgatggggaagctgaggctcacacatcctgcgggtggggacccagcctgaagaatgggctggtgtcacacagcattggagctgagactggggtctttagaatttcctaggtgggggcctgggaaccaacaggggctcaaggaaccaaggtgtccccacagtaagtggcactgtcaggtctaggatgggggtctcgggacccctggtcctggttctttccactgaattc

SEQ ID NO: 1012 - DNA, 138 bp, Homo sapiens
  source 1..138: mol_type = genomic DNA
ttcggttcccatggcagttggtgcaggagcaggtgcggcagacgatggcagaggccctaaaggtatggagcgatgtgacgccactcacctttactgaggtgcacgagggccgtgctgacatcatgatcgacttcgcca

SEQ ID NO: 1013 - DNA, 96 bp, Homo sapiens
  source 1..96: mol_type = genomic DNA
ggacgacctgccgtttgatgggcctgggggcatcctggcccatgccttcttccccaagactcaccgagaaggggatgtccacttcgactatgatga

SEQ ID NO: 1014 - DNA, 25 bp, Homo sapiens
  source 1..25: mol_type = genomic DNA
aggtggcagcccatgaatttggcca

SEQ ID NO: 1015 - DNA, 134 bp, Homo sapiens
  source 1..134: mol_type = genomic DNA
tacacctttcgctacccactgagtctcagcccagatgactgcaggggcgttcaacacctatatggccagccctggcccactgtcacctccaggaccccagccctgggcccccaggctgggatagacaccaatga

SEQ ID NO: 1016 - DNA, 34 bp, Homo sapiens
  source 1..34: mol_type = genomic DNA
actgtgactgcagcatatgccctcagcatgtgtc

SEQ ID NO: 1017 - DNA, 144 bp, Homo sapiens
  source 1..144: mol_type = genomic DNA
ccgaggcgagctctttttcttcaaagcgggctttgtgtggcgcctccgtgggggccagctgcagcccggctacccagcattggcctctcgccactggcagggactgcccagccctgtggacgctgccttcgaggatgcccaggg

SEQ ID NO: 1018 - DNA, 72 bp, Homo sapiens
  source 1..72: mol_type = genomic DNA
ggagcattgcagatgccagggacttcacaaatgaaggcacagcatgggaaacctgcgtgggttccagggcag

SEQ ID NO: 1019 - DNA, 180 bp, Homo sapiens
  source 1..180: mol_type = genomic DNA
tttgtcacagccaaatgccagtggaaggagcagccgcccaggcagccctctactgatgagagtaacctcacccgtgcactagtttacagagcattcactgccccagcttatcccaggcctcccgcttccctctgcgggtggggtgctgagcaggcattattggcctgcatgttttactga

SEQ ID NO: 1020 - DNA, 117 bp, Homo sapiens
  source 1..117: mol_type = genomic DNA
ctcagtactgggtgtacgacggtgaaaagccagtcctgggccccgcacccctcaccgagctgggcctggtgaggttcccggtccatgctgccttggtctggggtcccgagaagaaca

SEQ ID NO: 1021 - DNA, 87 bp, Homo sapiens
  source 1..87: mol_type = genomic DNA
agcacccggcgtgtagacagtcccgtgccccgcagggccactgactggagaggggtgccctctgagatcgacgctgccttccaggat

SEQ ID NO: 1022 - DNA, 56 bp, Homo sapiens
  source 1..56: mol_type = genomic DNA
gctatgcctacttcctgcgcggccgcctctactggaagtttgaccctgtgaaggtg

SEQ ID NO: 1023 - DNA, 41 bp, Homo sapiens
  source 1..41: mol_type = genomic DNA
tcctgacttctttggctgtgccgagcctgccaacactttcc

SEQ ID NO: 1024 - DNA, 206 bp, Homo sapiens
  source 1..206: mol_type = genomic DNA
ctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagagga

SEQ ID NO: 1025 - DNA, 118 bp, Homo sapiens
  source 1..118: mol_type = genomic DNA
gagtgtccttgctgtatccctgttgtgaggttccttccaggggctggcactgaagcaagggtgctggggccccatggccttcagccctggctgagcaactgggctgtagggcagggcc

SEQ ID NO: 1026 - DNA, 170 bp, Homo sapiens
  source 1..170: mol_type = genomic DNA
gccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactg

SEQ ID NO: 1027 - DNA, 235 bp, Homo sapiens
  source 1..235: mol_type = genomic DNA
ctgatgggtgtgctaattacactgatttaatcgatacccattgtatgtgaaacagtatatacaccatatttacaattatgtatcagtttaacatttaaaaaaacatttctaatataagtatctctcaaactgtggattaacttcttgatttatatttaaatatgaatcttgaggaaaatagtgaaaataaccatcttgatttagtgtatttctcccatatgtgaattgtatatac

SEQ ID NO: 1028 - DNA, 59 bp, Homo sapiens
  source 1..59: mol_type = genomic DNA
aacgatgccttgtgtcaagaagaaggcagattgggccttgcgctggattggggacaaag

SEQ ID NO: 1029 - DNA, 27 bp, Homo sapiens
  source 1..27: mol_type = genomic DNA
agctgcagctctcgccgctgaaggggc